(19) United States
(12) Patent Application Publication
(10) Pub. No.: US 2025/0121052 A1
(43) Pub. Date: Apr. 17, 2025

(54) SARS-COV-2 VACCINES

(71) Applicant: Gritstone bio, Inc., Emeryville, CA (US)

(72) Inventors: Leonid Gitlin, Foster City, CA (US); Karin Jooss, San Diego, CA (US); Sue-Jean Hong, Emeryville, CA (US); Ciaran Daniel Scallan, San Francisco, CA (US); Amy Rachel Rappaport, Daly City, CA (US); Christine Denise Palmer, Cambridge, MA (US); Minh Duc Cao, Belmont, MA (US)

(21) Appl. No.: 18/658,552

(22) Filed: May 8, 2024

Related U.S. Application Data
(63) Continuation of application No. PCT/US2022/079510, filed on Nov. 8, 2022.
(60) Provisional application No. 63/379,896, filed on Oct. 17, 2022.
(60) Provisional application No. 63/370,373, filed on Aug. 3, 2022.
(60) Provisional application No. 63/346,769, filed on May 27, 2022.
(60) Provisional application No. 63/322,142, filed on Mar. 21, 2022.
(60) Provisional application No. 63/306,022, filed on Feb. 2, 2022.
(60) Provisional application No. 63/295,449, filed on Dec. 30, 2021.
(60) Provisional application No. 63/265,991, filed on Dec. 23, 2021.
(60) Provisional application No. 63/277,105, filed on Nov. 8, 2021.

(51) Int. Cl. A61K 39/215; A61K 9/51; A61K 39/00; A61P 37/04

(52) U.S. Cl. CPC A61K 39/215; A61K 9/5123; A61P 37/04; A61K 2039/5256

(57) ABSTRACT

Disclosed herein are vaccine compositions that include SARS-CoV-2 MHC epitope-encoding cassettes and/or full-length SARS-CoV-2 proteins. Also disclosed are nucleotides, cells, and methods associated with the compositions including their use as vaccines.

### CROSS REFERENCE TO RELATED APPLICATIONS

This application is a Continuation of International Patent Application No. PCT/US2022/079510, filed Nov. 8, 2022, which claims the benefit of U.S. Provisional Application Nos. 63/277,105 filed Nov. 8, 2021, 63/295,449 filed Dec. 30, 2021, 63/306,022 filed Feb. 2, 2022, 63/322,142 filed Mar. 21, 2022, 63/346,769 filed May 27, 2022, 63/370,373 filed Aug. 3, 2022, 63/379,896 filed Oct. 17, 2022, and 63/265,991 filed Dec. 23, 2021, each of which is hereby incorporated in its entirety by reference for all purposes.

### SEQUENCE LISTING

The instant application contains a Sequence Listing which has been submitted electronically in xml format and is hereby incorporated by reference in its entirety. Said .xml copy, created on 6 Jan. 2023, is named GSO-102WO2_SL and is 40,205,856 bytes in size.

### BACKGROUND

Severe acute respiratory syndrome corona virus 2 (SARS-CoV-2) is the virus strain responsible for the Coronavirus Disease 2019 (Covid-19) pandemic. As of Dec. 21, 2021, the virus has infected over 275 million people and caused about 5.4 million deaths worldwide. A CD8+ T cell response may be important for COVID-19 for two reasons in a coronavirus context. First is the recurrent observation in pre-clinical models that SARS vaccines that only stimulate antibody responses are often associated with pulmonary inflammation, independent of viral clearance. This has been observed in both rodents and non-human primates (NHP), and the current consensus is that it is caused by an imbalanced immune response, and is likely to be solved by using vaccines that drive a balanced antibody and CD8+ T cell (Th1) response (Consensus considerations on the assessment of the risk of disease enhancement with COVID-19 vaccines: Outcome of a Coalition for Epidemic Preparedness Innovations (CEPI)/Brighton Collaboration (BC) scientific working meeting, Mar. 12-13, 2020). Secondly, coronaviruses are evidently mutating frequently and crossing from animal reservoirs into humans, with three epidemics/pandemics over the last 18 years (SARS in 2002, MERS in 2012, now COVID-19). Antibody responses are often against highly mutable proteins (such as the Spike protein of SARS-CoV-2) which change significantly between strains and isolates, whereas T cell epitopes often derive from more evolutionarily conserved proteins. T cell memory is also generally more durable than B cell memory and thus CD8+ T memory against SARS-CoV-2 may provide longer, and better protection against future SARS variants. Many vaccines have demonstrated an ability to drive antibody responses in NHP and humans, but commonly used modalities such as protein/peptide and mRNA vaccines have not stimulated meaningful CD8+ T cell responses in these species.

An additional question for antigen vaccine design in infectious disease settings is which of the many proteins present generate the “best” therapeutic antigens, e.g., antigens that can stimulate immunity.

In addition to the challenges of current antigen prediction methods certain challenges also exist with the available vector systems that can be used for antigen delivery in humans, many of which are derived from humans. For example, many humans have pre-existing immunity to human viruses as a result of previous natural exposure, and this immunity can be a major obstacle to the use of recombinant human viruses for antigen delivery in vaccination strategies, such as in cancer treatment or vaccinations against infectious diseases.

While some progress has been made in vaccinations strategies addressing the above problems, improvements are still needed, particularly for clinical applications, such as improved vaccine potency and efficacy, such as the need for a SARS-CoV-2 vaccine that stimulates balanced B and T cell immunity in humans, including the elderly.

### SUMMARY

Provided for herein is a composition for delivery of a self-amplifying alphavirus-based expression system, wherein the composition for delivery of the self-amplifying alphavirus-based expression system comprises: (A) the self-amplifying alphavirus-based expression system, wherein the self-amplifying alphavirus-based expression system comprises one or more vectors, wherein the one or more vectors comprises: (a) an RNA alphavirus backbone, wherein the RNA alphavirus backbone comprises: (i) at least one promoter nucleotide sequence, and (ii) at least one polyadenylation (poly(A)) sequence; and (b) an antigen cassette, wherein the antigen cassette is inserted into the vector backbone, and wherein the antigen cassette comprises: (i) at least one SARS-CoV-2 derived nucleic acid sequence encoding an immunogenic polypeptide, wherein the immunogenic polypeptide comprises:
at least one MHC class I epitope comprising a polypeptide sequence as set forth in Table A,
at least one MHC class II epitope comprising a polypeptide sequence as set forth in Table B,
at least one MHC class I epitope comprising a polypeptide sequence as set forth in Table C, optionally wherein the at least one MHC I epitope is present in a concatenated polypeptide sequence as set forth in SEQ ID NO:57 or SEQ ID NO:58,
at least one polypeptide sequence as set forth in Table 7, or an epitope-containing fragment thereof, optionally wherein the at least one polypeptide sequence is present in a concatenated polypeptide sequence as set forth in SEQ ID NO:92,
at least one polypeptide sequence as set forth in Table 9A, Table 9B, or Table 9C, or an epitope-containing fragment thereof, optionally wherein the at least one polypeptide sequence is present in a concatenated polypeptide comprising each of the sequences set forth in Table 9A, Table 9B, or Table 9C, optionally wherein the concatenated polypeptide comprises the order of sequences set forth in Table 9A, Table 9B, or Table 9C,
at least one MHC class I epitope comprising a polypeptide sequence as set forth in Table A and/or Table C or MHC class II epitope comprising a polypeptide sequence as set forth in Table B, wherein the encoded SARS-CoV-2 immunogenic polypeptide is conserved between SARS-CoV-2 and a Coronavirus species and/or sub-species other than SARS-CoV-2, optionally wherein the Coronavirus species and/or sub-species other than SARS-CoV-2 is Severe acute respiratory syndrome (SARS) and/or Middle East respiratory syndrome (MERS),
one or more validated epitopes and/or at least 4, 5, 6, or 7 predicted epitopes, wherein at least 85%, 90%, or 95% of a population carries at least one HLA validated to present at least one of the one or more validated epitopes and/or at least one HLA predicted to present each of the at least 4, 5, 6, or 7 predicted epitopes,
a SARS-CoV-2 Spike protein comprising a Spike polypeptide sequence as set forth in SEQ ID NO:59 or an epitope-containing fragment thereof, optionally wherein the Spike polypeptide comprises a D614G mutation with reference to SEQ ID NO:59, and optionally wherein the Spike polypeptide is encoded by the nucleotide sequence shown in SEQ ID NO:79, SEQ ID NO:83, SEQ ID NO:85, or SEQ ID NO:87,
a SARS-CoV-2 modified Spike protein comprising a mutation selected from the group consisting of: a Spike R682 mutation, a Spike R815 mutation, a Spike K986P mutation, a Spike V987P mutation, and combinations thereof with reference to the Spike polypeptide sequence as set forth in SEQ ID NO:59, and optionally wherein the modified Spike protein comprises a polypeptide sequence as set forth in SEQ ID NO:60 or SEQ ID NO:90 or an epitope-containing fragment thereof,
a SARS-CoV-2 Membrane protein comprising a Membrane polypeptide sequence as set forth in SEQ ID NO:61 or an epitope-containing fragment thereof,
a SARS-CoV-2 Nucleocapsid protein comprising a Nucleocapsid polypeptide sequence as set forth in SEQ ID NO:62 or an epitope-containing fragment thereof,
a SARS-CoV-2 Envelope protein comprising an Envelope polypeptide sequence as set forth in SEQ ID NO:63 or an epitope-containing fragment thereof,
a variant of any of the above comprising a mutation found in 1% or greater of SARS-CoV-2 subtypes, optionally wherein the variant comprises a SARS-CoV-2 variant shown in Table 1, and/or optionally wherein the variant comprises a SARS-CoV-2 variant Spike protein comprising a Spike D614G mutation with reference to the Spike polypeptide sequence as set forth in SEQ ID NO:59, a SARS-CoV-2 variant Spike protein corresponding to a B.1.351 SARS-CoV-2 isolate optionally comprising the Spike polypeptide sequence as set forth in SEQ ID NO:112 or subvariant, a SARS-CoV-2 variant Spike protein corresponding to a B.1.1.7 SARS-CoV-2 isolate optionally comprising the Spike polypeptide sequence as set forth in SEQ ID NO:110, a SARS-CoV-2 variant Spike protein corresponding to a B.1.1.529 SARS-CoV-2 isolate optionally comprising the Spike polypeptide sequence as set forth in SEQ ID NO: 27974 and/or is encoded by nucleotides 9714-13,526 of the nucleotide sequence set forth in SEQ ID NO: 27976, or a SARS-CoV-2 variant Spike protein corresponding to a B.1.1.529 BA5 SARS-CoV-2 subvariant optionally comprising the Spike polypeptide sequence as set forth in SEQ ID NO: 27978 and/or encoded by SEQ ID NO: 27980,
or combinations thereof; and
   wherein the immunogenic polypeptide optionally comprises a N-terminal linker and/or a C-terminal linker; (ii) optionally, a second promoter nucleotide sequence operably linked to the SARS-CoV-2 derived nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, optionally wherein the exogenous poly(A) sequence comprises an SV40 poly(A) signal sequence or a Bovine Growth Hormone (BGH) poly(A) signal sequence; and (B) a lipid-nanoparticle (LNP), wherein the LNP encapsulates the self-amplifying alphavirus-based expression system, and wherein the composition comprises at least 10 μg of each of the one or more vectors.

In some aspects, the composition for delivery of the self-amplifying alphavirus-based expression system comprises at least 30 μg of each of the one or more vectors. In some aspects, the composition for delivery of the self-amplifying alphavirus-based expression system comprises 30 μg or less of each of the one or more vectors. In some aspects, the composition for delivery of the self-amplifying alphavirus-based expression system comprises between 10-30 μg or between 10-100 μg of each of the one or more vectors. In some aspects, the composition for delivery of the self-amplifying alphavirus-based expression system comprises at least 10 μg total of the one or more vectors combined. In some aspects, the composition for delivery of the self-amplifying alphavirus-based expression system comprises at least 30 μg total of the one or more vectors combined. In some aspects, the composition for delivery of the self-amplifying alphavirus-based expression system comprises 30 μg or less total of the one or more vectors combined. In some aspects, the composition for delivery of the self-amplifying alphavirus-based expression system comprises between 10-30 μg or between 10-100 μg total of the one or more vectors combined.

In some aspects, the one or more vectors of wherein the self-amplifying alphavirus-based expression system is at a concentration of 1 mg/mL. In some aspects, the RNA alphavirus backbone comprises one or more elements obtained from the sequence of SEQ ID NO:3 or SEQ ID NO:5, optionally wherein the one or more elements are selected from the group consisting of the sequences necessary for nonstructural protein-mediated amplification, the 26S promoter nucleotide sequence, the poly(A) sequence, and the nsP1-4 genes of the sequence set forth in SEQ ID NO:3 or SEQ ID NO:5, optionally the RNA alphavirus backbone comprises the sequence set forth in the sequences selected from the group consisting of SEQ ID NOs:6-9.

In some aspects, the self-amplifying alphavirus-based expression system comprises a vector selected from the group of sequences consisting of: SEQ ID NO: 27983, SEQ ID NO:27981, SEQ ID NO:27982, SEQ ID NO: 27976, and SEQ ID NO: 27976 with Spike encoding sequences substituted with the sequence set forth in SEQ ID NO:27980.

Also provided for herein is a method for stimulating an immune response in a subject, the method comprising administering to the subject the composition for delivery of the self-amplifying alphavirus-based expression systems provided herein.

In some aspects, the method comprises administering at least two doses of the composition for delivery of the self-amplifying alphavirus-based expression system. In some aspects, the at least two doses comprises a priming dose and at least one boosting dose. In some aspects, the at least two doses are administered on days 1 and day 28 or later. In some aspects, day 28 or later comprises day 29 or later. In some aspects, the at least two doses are administered on days 1 and on or after week 4. In some aspects, the at least two doses are administered on days 1 and between day 28 to 113. In some aspects, the at least two doses are administered on days 1 and day 113 or later.

In some aspects, the at least two doses comprise the same antigen cassette.

In some aspects, the method further comprises administration of a chimpanzee adenovirus (ChAdV)-based expression system, wherein the composition for delivery of the ChAdV-based expression system comprises: the ChAdV-based expression system, wherein the ChAdV-based expression system comprises a viral particle comprising a ChAdV vector, wherein the ChAdV vector comprises: (a) a ChAdV backbone, wherein the ChAdV backbone comprises: (i) at least one promoter nucleotide sequence, and (ii) at least one polyadenylation (poly(A)) sequence; and (b) an antigen cassette, wherein the antigen cassette is inserted into the vector backbone, and wherein the antigen cassette comprises at least one SARS-CoV-2 derived nucleic acid sequence encoding an immunogenic polypeptide.

In some aspects, the ChAdV-based expression system is administered as a priming dose. In some aspects, the antigen cassette of the ChAdV-based expression system is the same as the antigen cassette of the self-amplifying alphavirus-based expression system.

Also provided for herein is a composition for delivery of a chimpanzee adenovirus (ChAdV)-based expression system, wherein the composition for delivery of the ChAdV-based expression system comprises: the ChAdV-based expression system, wherein the ChAdV-based expression system comprises a viral particle comprising a ChAdV vector, wherein the ChAdV vector comprises: (a) a ChAdV backbone, wherein the ChAdV backbone comprises: (i) at least one promoter nucleotide sequence, and (ii) at least one polyadenylation (poly(A)) sequence; and (b) an antigen cassette, wherein the antigen cassette is inserted into the vector backbone, and wherein the antigen cassette comprises: (i) at least one SARS-CoV-2 derived nucleic acid sequence encoding an immunogenic polypeptide, wherein the immunogenic polypeptide comprises:
at least one MHC class I epitope comprising a polypeptide sequence as set forth in Table A,
at least one MHC class II epitope comprising a polypeptide sequence as set forth in Table B,
at least one MHC class I epitope comprising a polypeptide sequence as set forth in Table C, optionally wherein the at least one MHC I epitope is present in a concatenated polypeptide sequence as set forth in SEQ ID NO:57 or SEQ ID NO:58,
at least one polypeptide sequence as set forth in Table 7, or an epitope-containing fragment thereof, optionally wherein the at least one polypeptide sequence is present in a concatenated polypeptide sequence as set forth in SEQ ID NO:92,
at least one polypeptide sequence as set forth in Table 9A, Table 9B, or Table 9C, or an epitope-containing fragment thereof, optionally wherein the at least one polypeptide sequence is present in a concatenated polypeptide comprising each of the sequences set forth in Table 9A, Table 9B, or Table 9C, optionally wherein the concatenated polypeptide comprises the order of sequences set forth in Table 9A, Table 9B, or Table 9C,
at least one MHC class I epitope comprising a polypeptide sequence as set forth in Table A and/or Table C or MHC class II epitope comprising a polypeptide sequence as set forth in Table B, wherein the encoded SARS-CoV-2 immunogenic polypeptide is conserved between SARS-CoV-2 and a Coronavirus species and/or sub-species other than SARS-CoV-2, optionally wherein the Coronavirus species and/or sub-species other than SARS-CoV-2 is Severe acute respiratory syndrome (SARS) and/or Middle East respiratory syndrome (MERS),
one or more validated epitopes and/or at least 4, 5, 6, or 7 predicted epitopes, wherein at least 85%, 90%, or 95% of a population carries at least one HLA validated to present at least one of the one or more validated epitopes and/or at least one HLA predicted to present each of the at least 4, 5, 6, or 7 predicted epitopes,
a SARS-CoV-2 Spike protein comprising a Spike polypeptide sequence as set forth in SEQ ID NO:59 or an epitope-containing fragment thereof, optionally wherein the Spike polypeptide comprises a D614G mutation with reference to SEQ ID NO:59, and optionally wherein the Spike polypeptide is encoded by the nucleotide sequence shown in SEQ ID NO:79, SEQ ID NO:83, SEQ ID NO:85, or SEQ ID NO:87,
a SARS-CoV-2 modified Spike protein comprising a mutation selected from the group consisting of: a Spike R682 mutation, a Spike R815 mutation, a Spike K986P mutation, a Spike V987P mutation, and combinations thereof with reference to the Spike polypeptide sequence as set forth in SEQ ID NO:59, and optionally wherein the modified Spike protein comprises a polypeptide sequence as set forth in SEQ ID NO:60 or SEQ ID NO:90 or an epitope-containing fragment thereof,
a SARS-CoV-2 Membrane protein comprising a Membrane polypeptide sequence as set forth in SEQ ID NO:61 or an epitope-containing fragment thereof,
a SARS-CoV-2 Nucleocapsid protein comprising a Nucleocapsid polypeptide sequence as set forth in SEQ ID NO:62 or an epitope-containing fragment thereof,
a SARS-CoV-2 Envelope protein comprising an Envelope polypeptide sequence as set forth in SEQ ID NO:63 or an epitope-containing fragment thereof,
a variant of any of the above comprising a mutation found in 1% or greater of SARS-CoV-2 subtypes, optionally wherein the variant comprises a SARS-CoV-2 variant shown in Table 1, and/or optionally wherein the variant comprises a SARS-CoV-2 variant Spike protein comprising a Spike D614G mutation with reference to the Spike polypeptide sequence as set forth in SEQ ID NO:59, a SARS-CoV-2 variant Spike protein corresponding to a B.1.351 SARS-CoV-2 isolate optionally comprising the Spike polypeptide sequence as set forth in SEQ ID NO:112 or subvariant, a SARS-CoV-2 variant Spike protein corresponding to a B.1.1.7 SARS-CoV-2 isolate optionally comprising the Spike polypeptide sequence as set forth in SEQ ID NO:110, a SARS-CoV-2 variant Spike protein corresponding to a B.1.1.529 SARS-CoV-2 isolate optionally comprising the Spike polypeptide sequence as set forth in SEQ ID NO: 27974 and/or is encoded by nucleotides 9714-13,526 of the nucleotide sequence set forth in SEQ ID NO: 27976, or a SARS-CoV-2 variant Spike protein corresponding to a B.1.1.529 BA5 SARS-CoV-2 subvariant optionally comprising the Spike polypeptide sequence as set forth in SEQ ID NO: 27978 and/or encoded by SEQ ID NO: 27980,
or combinations thereof; and
   wherein the immunogenic polypeptide optionally comprises a N-terminal linker and/or a C-terminal linker; (ii) optionally, a second promoter nucleotide sequence operably linked to the SARS-CoV-2 derived nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, optionally wherein the exogenous poly(A) sequence comprises an SV40 poly(A) signal sequence or a Bovine Growth Hormone (BGH) poly(A) signal sequence, and wherein the cassette is operably linked to the at least one promoter nucleotide sequence and the at least one poly(A) sequence, and wherein the composition comprises 1×10¹² or less of the viral particles.

In some aspects, the composition for delivery of the ChAdV-based expression system comprises at least 1×10¹¹ of the viral particles. In some aspects, the composition for delivery of the ChAdV-based expression system comprises between 1×10¹¹ and 1×10¹². In some aspects, the composition for delivery of the ChAdV-based expression system comprises 1×10¹¹, 3×10¹¹, or 1×10¹² of the viral particles. In some aspects, the viral particles are at a concentration of 5×10¹¹ vp/mL.

In some aspects, the ChAdV backbone comprises at least nucleotides 2 to 36,518 of the sequence set forth in SEQ ID NO:1, wherein the nucleotides 2 to 36,518 lack: (1) nucleotides 577 to 3403 of the sequence shown in SEQ ID NO:1 corresponding to an E1 deletion; (2) nucleotides 27,125 to 31,825 of the sequence shown in SEQ ID NO:1 corresponding to an E3 deletion; and (3) optionally nucleotides 34,916 to 35,642 of the sequence shown in SEQ ID NO:1 corresponding to a partial E4 deletion; optionally wherein the antigen cassette is inserted within the E1 deletion.

Also provided for herein is a method for stimulating an immune response in a subject, the method comprising administering to the subject the composition for delivery of the ChAdV-based expression systems described herein.

In some aspects, the the ChAdV-based expression system is administered as a priming dose. In some aspects, the method further comprises administration of a composition for delivery of a self-amplifying alphavirus-based expression system, wherein the composition for delivery of the self-amplifying alphavirus-based expression system comprises: (A) the self-amplifying alphavirus-based expression system, wherein the self-amplifying alphavirus-based expression system comprises one or more vectors, wherein the one or more vectors comprises: (a) an RNA alphavirus backbone, wherein the RNA alphavirus backbone comprises: (i) at least one promoter nucleotide sequence, and (ii) at least one polyadenylation (poly(A)) sequence; and (b) an antigen cassette, wherein the antigen cassette is inserted into the vector backbone, and wherein the antigen cassette comprises at least one SARS-CoV-2 derived nucleic acid sequence encoding an immunogenic polypeptide.

In some aspects, the antigen cassette of the ChAdV-based expression system is the same as the antigen cassette of the self-amplifying alphavirus-based expression system.

In some aspects, the composition for delivery of the expression system is formulated in a pharmaceutical composition comprising a pharmaceutically acceptable carrier.

Also provided herein is a kit comprising the composition for delivery of the expression systems provided herein, and instructions for use.

Also provided for herein is a method for stimulating an immune response in a subject, the method comprising administering to the subject a composition for delivery of a self-amplifying alphavirus-based expression system wherein the composition for delivery of the self-amplifying alphavirus-based expression system comprises: (A) the self-amplifying alphavirus-based expression system, wherein the self-amplifying alphavirus-based expression system comprises one or more vectors, wherein the one or more vectors comprises: (a) an RNA alphavirus backbone, wherein the RNA alphavirus backbone comprises: (i) at least one promoter nucleotide sequence, and (ii) at least one polyadenylation (poly(A)) sequence; and (b) an antigen cassette, wherein the antigen cassette is inserted into the vector backbone, and wherein the antigen cassette comprises: (i) at least one SARS-CoV-2 derived nucleic acid sequence encoding an immunogenic polypeptide, wherein the immunogenic polypeptide comprises:
at least one MHC class I epitope comprising a polypeptide sequence as set forth in Table A,
at least one MHC class II epitope comprising a polypeptide sequence as set forth in Table B,
at least one MHC class I epitope comprising a polypeptide sequence as set forth in Table C, optionally wherein the at least one MHC I epitope is present in a concatenated polypeptide sequence as set forth in SEQ ID NO:57 or SEQ ID NO:58,
at least one polypeptide sequence as set forth in Table 7, or an epitope-containing fragment thereof, optionally wherein the at least one polypeptide sequence is present in a concatenated polypeptide sequence as set forth in SEQ ID NO:92,
at least one polypeptide sequence as set forth in Table 9A, Table 9B, or Table 9C, or an epitope-containing fragment thereof, optionally wherein the at least one polypeptide sequence is present in a concatenated polypeptide comprising each of the sequences set forth in Table 9A, Table 9B, or Table 9C, optionally wherein the concatenated polypeptide comprises the order of sequences set forth in Table 9A, Table 9B, or Table 9C,
at least one MHC class I epitope comprising a polypeptide sequence as set forth in Table A and/or Table C or MHC class II epitope comprising a polypeptide sequence as set forth in Table B, wherein the encoded SARS-CoV-2 immunogenic polypeptide is conserved between SARS-CoV-2 and a Coronavirus species and/or sub-species other than SARS-CoV-2, optionally wherein the Coronavirus species and/or sub-species other than SARS-CoV-2 is Severe acute respiratory syndrome (SARS) and/or Middle East respiratory syndrome (MERS),
one or more validated epitopes and/or at least 4, 5, 6, or 7 predicted epitopes, wherein at least 85%, 90%, or 95% of a population carries at least one HLA validated to present at least one of the one or more validated epitopes and/or at least one HLA predicted to present each of the at least 4, 5, 6, or 7 predicted epitopes,
a SARS-CoV-2 Spike protein comprising a Spike polypeptide sequence as set forth in SEQ ID NO:59 or an epitope-containing fragment thereof, optionally wherein the Spike polypeptide comprises a D614G mutation with reference to SEQ ID NO:59, and optionally wherein the Spike polypeptide is encoded by the nucleotide sequence shown in SEQ ID NO:79, SEQ ID NO:83, SEQ ID NO:85, or SEQ ID NO:87,
a SARS-CoV-2 modified Spike protein comprising a mutation selected from the group consisting of: a Spike R682 mutation, a Spike R815 mutation, a Spike K986P mutation, a Spike V987P mutation, and combinations thereof with reference to the Spike polypeptide sequence as set forth in SEQ ID NO:59, and optionally wherein the modified Spike protein comprises a polypeptide sequence as set forth in SEQ ID NO:60 or SEQ ID NO:90 or an epitope-containing fragment thereof,
a SARS-CoV-2 Membrane protein comprising a Membrane polypeptide sequence as set forth in SEQ ID NO:61 or an epitope-containing fragment thereof,
a SARS-CoV-2 Nucleocapsid protein comprising a Nucleocapsid polypeptide sequence as set forth in SEQ ID NO:62 or an epitope-containing fragment thereof,
a SARS-CoV-2 Envelope protein comprising an Envelope polypeptide sequence as set forth in SEQ ID NO:63 or an epitope-containing fragment thereof,
a variant of any of the above comprising a mutation found in 1% or greater of SARS-CoV-2 subtypes, optionally wherein the variant comprises a SARS-CoV-2 variant shown in Table 1, and/or optionally wherein the variant comprises a SARS-CoV-2 variant Spike protein comprising a Spike D614G mutation with reference to the Spike polypeptide sequence as set forth in SEQ ID NO:59, a SARS-CoV-2 variant Spike protein corresponding to a B.1.351 SARS-CoV-2 isolate optionally comprising the Spike polypeptide sequence as set forth in SEQ ID NO:112 or subvariant, a SARS-CoV-2 variant Spike protein corresponding to a B.1.1.7 SARS-CoV-2 isolate optionally comprising the Spike polypeptide sequence as set forth in SEQ ID NO:110, a SARS-CoV-2 variant Spike protein corresponding to a B.1.1.529 SARS-CoV-2 isolate optionally comprising the Spike polypeptide sequence as set forth in SEQ ID NO: 27974 and/or is encoded by nucleotides 9714-13,526 of the nucleotide sequence set forth in SEQ ID NO: 27976, or a SARS-CoV-2 variant Spike protein corresponding to a B.1.1.529 BA5 SARS-CoV-2 subvariant optionally comprising the Spike polypeptide sequence as set forth in SEQ ID NO: 27978 and/or encoded by SEQ ID NO: 27980,
or combinations thereof; and
   wherein the immunogenic polypeptide optionally comprises a N-terminal linker and/or a C-terminal linker; (ii) optionally, a second promoter nucleotide sequence operably linked to the SARS-CoV-2 derived nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, optionally wherein the exogenous poly(A) sequence comprises an SV40 poly(A) signal sequence or a Bovine Growth Hormone (BGH) poly(A) signal sequence; and (B) a lipid-nanoparticle (LNP), wherein the LNP encapsulates the self-amplifying alphavirus-based expression system, and wherein the composition comprises at least 10 μg of each of the one or more vectors.

Also provided for herein is a method for stimulating an immune response in a subject, the method comprising administering to the subject a composition for delivery of the ChAdV-based expression system, wherein the composition for delivery of the ChAdV-based expression system comprises: the ChAdV-based expression system, wherein the ChAdV-based expression system comprises a viral particle comprising a ChAdV vector, wherein the ChAdV vector comprises: (a) a ChAdV backbone, wherein the ChAdV backbone comprises: (i) at least one promoter nucleotide sequence, and (ii) at least one polyadenylation (poly(A)) sequence; and (b) an antigen cassette, wherein the antigen cassette is inserted into the vector backbone, and wherein the antigen cassette comprises: (i) at least one SARS-CoV-2 derived nucleic acid sequence encoding an immunogenic polypeptide, wherein the immunogenic polypeptide comprises:
at least one MHC class I epitope comprising a polypeptide sequence as set forth in Table A,
at least one MHC class II epitope comprising a polypeptide sequence as set forth in Table B,
at least one MHC class I epitope comprising a polypeptide sequence as set forth in Table C, optionally wherein the at least one MHC I epitope is present in a concatenated polypeptide sequence as set forth in SEQ ID NO:57 or SEQ ID NO:58,
at least one polypeptide sequence as set forth in Table 7, or an epitope-containing fragment thereof, optionally wherein the at least one polypeptide sequence is present in a concatenated polypeptide sequence as set forth in SEQ ID NO:92,
at least one polypeptide sequence as set forth in Table 9A, Table 9B, or Table 9C, or an epitope-containing fragment thereof, optionally wherein the at least one polypeptide sequence is present in a concatenated polypeptide comprising each of the sequences set forth in Table 9A, Table 9B, or Table 9C, optionally wherein the concatenated polypeptide comprises the order of sequences set forth in Table 9A, Table 9B, or Table 9C,
at least one MHC class I epitope comprising a polypeptide sequence as set forth in Table A and/or Table C or MHC class II epitope comprising a polypeptide sequence as set forth in Table B, wherein the encoded SARS-CoV-2 immunogenic polypeptide is conserved between SARS-CoV-2 and a Coronavirus species and/or sub-species other than SARS-CoV-2, optionally wherein the Coronavirus species and/or sub-species other than SARS-CoV-2 is Severe acute respiratory syndrome (SARS) and/or Middle East respiratory syndrome (MERS),
one or more validated epitopes and/or at least 4, 5, 6, or 7 predicted epitopes, wherein at least 85%, 90%, or 95% of a population carries at least one HLA validated to present at least one of the one or more validated epitopes and/or at least one HLA predicted to present each of the at least 4, 5, 6, or 7 predicted epitopes,
a SARS-CoV-2 Spike protein comprising a Spike polypeptide sequence as set forth in SEQ ID NO:59 or an epitope-containing fragment thereof, optionally wherein the Spike polypeptide comprises a D614G mutation with reference to SEQ ID NO:59, and optionally wherein the Spike polypeptide is encoded by the nucleotide sequence shown in SEQ ID NO:79, SEQ ID NO:83, SEQ ID NO:85, or SEQ ID NO:87,
a SARS-CoV-2 modified Spike protein comprising a mutation selected from the group consisting of: a Spike R682 mutation, a Spike R815 mutation, a Spike K986P mutation, a Spike V987P mutation, and combinations thereof with reference to the Spike polypeptide sequence as set forth in SEQ ID NO:59, and optionally wherein the modified Spike protein comprises a polypeptide sequence as set forth in SEQ ID NO:60 or SEQ ID NO:90 or an epitope-containing fragment thereof,
a SARS-CoV-2 Membrane protein comprising a Membrane polypeptide sequence as set forth in SEQ ID NO:61 or an epitope-containing fragment thereof,
a SARS-CoV-2 Nucleocapsid protein comprising a Nucleocapsid polypeptide sequence as set forth in SEQ ID NO:62 or an epitope-containing fragment thereof,
a SARS-CoV-2 Envelope protein comprising an Envelope polypeptide sequence as set forth in SEQ ID NO:63 or an epitope-containing fragment thereof,
a variant of any of the above comprising a mutation found in 1% or greater of SARS-CoV-2 subtypes, optionally wherein the variant comprises a SARS-CoV-2 variant shown in Table 1, and/or optionally wherein the variant comprises a SARS-CoV-2 variant Spike protein comprising a Spike D614G mutation with reference to the Spike polypeptide sequence as set forth in SEQ ID NO:59, a SARS-CoV-2 variant Spike protein corresponding to a B.1.351 SARS-CoV-2 isolate optionally comprising the Spike polypeptide sequence as set forth in SEQ ID NO:112 or subvariant, a SARS-CoV-2 variant Spike protein corresponding to a B.1.1.7 SARS-CoV-2 isolate optionally comprising the Spike polypeptide sequence as set forth in SEQ ID NO:110, a SARS-CoV-2 variant Spike protein corresponding to a B.1.1.529 SARS-CoV-2 isolate optionally comprising the Spike polypeptide sequence as set forth in SEQ ID NO: 27974 and/or is encoded by nucleotides 9714-13,526 of the nucleotide sequence set forth in SEQ ID NO: 27976, or a SARS-CoV-2 variant Spike protein corresponding to a B.1.1.529 BA5 SARS-CoV-2 subvariant optionally comprising the Spike polypeptide sequence as set forth in SEQ ID NO: 27978 and/or encoded by SEQ ID NO: 27980,
or combinations thereof; and
   wherein the immunogenic polypeptide optionally comprises a N-terminal linker and/or a C-terminal linker; (ii) optionally, a second promoter nucleotide sequence operably linked to the SARS-CoV-2 derived nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, optionally wherein the exogenous poly(A) sequence comprises an SV40 poly(A) signal sequence or a Bovine Growth Hormone (BGH) poly(A) signal sequence, and wherein the cassette is operably linked to the at least one promoter nucleotide sequence and the at least one poly(A) sequence, and wherein the composition comprises 1×10¹² or less of the viral particles.

Also provided for herein is a method for stimulating an immune response in a subject, the method comprising administering to the subject a composition for delivery of a self-amplifying alphavirus-based expression system and administering to the subject a composition for delivery of a chimpanzee adenovirus (ChAdV)-based expression system, and wherein either: a. the composition for delivery of the ChAdV-based expression system comprises the ChAdV-based expression system, wherein the ChAdV-based expression system comprises a viral particle comprising a ChAdV vector, and wherein the composition comprises 1×10¹² or less of the viral particles, b. wherein the composition for delivery of the self-amplifying alphavirus-based expression system comprises the self-amplifying alphavirus-based expression system, wherein the self-amplifying alphavirus-based expression system comprises one or more vectors, and wherein the composition comprises at least 10 μg of each of the one or more vectors, or c. the composition for delivery of the ChAdV-based expression system comprises the ChAdV-based expression system, wherein the ChAdV-based expression system comprises a viral particle comprising a ChAdV vector, and wherein the composition comprises 1×10¹² or less of the viral particles and wherein the composition for delivery of the self-amplifying alphavirus-based expression system comprises the self-amplifying alphavirus-based expression system, wherein the self-amplifying alphavirus-based expression system comprises one or more vectors, and wherein the composition comprises at least 10 μg of each of the one or more vectors.

In some aspects, the composition for delivery of the ChAdV-based expression system is administered as a priming dose and the composition for delivery of the self-amplifying alphavirus-based expression system is administered as one or more boosting doses.

Also provided for herein is a composition for delivery of an antigen expression system, comprising: the antigen expression system, wherein the antigen expression system comprises: (a) optionally, one or more vectors, the one or more vectors comprising: a vector backbone, wherein the vector backbone comprises: (i) at least one promoter nucleotide sequence, and (ii) at least one polyadenylation (poly(A)) sequence; and (b) an antigen cassette, optionally wherein the antigen cassette is inserted into the vector backbone when present, and wherein the antigen cassette comprises: (i) a SARS-CoV-2 derived nucleic acid sequence encoding an immunogenic polypeptide, wherein the immunogenic polypeptide comprises a SARS-CoV-2 variant Spike protein corresponding to a B.1.1.529 SARS-CoV-2 isolate optionally comprising the Spike polypeptide sequence as set forth in SEQ ID NO: 27974 and/or is encoded by nucleotides 9714-13,526 of the nucleotide sequence set forth in SEQ ID NO: 27976, or a SARS-CoV-2 variant Spike protein corresponding to a B.1.1.529 BA5 SARS-CoV-2 subvariant optionally comprising the Spike polypeptide sequence as set forth in SEQ ID NO: 27978 and/or encoded by SEQ ID NO: 27980, optionally wherein (a) the B.1.1.529 isolate Spike protein or fragment comprises a mutation selected from the group consisting of: a Spike R679 mutation, a Spike R680 mutation, a Spike R682 mutation, a Spike K983P mutation, a Spike V984P mutation, and combinations thereof with reference to the Spike polypeptide sequence as set forth in SEQ ID NO:27977 or (b) the B.1.1.529 BA5 subvariant Spike protein or fragment comprises a mutation selected from the group consisting of: a Spike R677 mutation, a Spike R678 mutation, a Spike R680 mutation, a Spike K981P mutation, a Spike V982P mutation, and combinations thereof with reference to the Spike polypeptide sequence as set forth in SEQ ID NO: 27979, and optionally wherein the antigen cassette further comprises at least one additional SARS-CoV-2 derived nucleic acid sequence encoding at least one additional immunogenic polypeptide, wherein the at least one additional immunogenic polypeptide comprises:
at least one MHC class I epitope comprising a polypeptide sequence as set forth in Table A,
at least one MHC class II epitope comprising a polypeptide sequence as set forth in Table B,
at least one MHC class I epitope comprising a polypeptide sequence as set forth in Table C, optionally wherein the at least one MHC I epitope is present in a concatenated polypeptide sequence as set forth in SEQ ID NO:57 or SEQ ID NO:58,
at least one polypeptide sequence as set forth in Table 6, or an epitope-containing fragment thereof, optionally wherein the at least one polypeptide sequence is present in a concatenated polypeptide sequence as set forth in SEQ ID NO:92,
at least one polypeptide sequence as set forth in Table 9A, Table 9B, or Table 9C, or an epitope-containing fragment thereof, optionally wherein the at least one polypeptide sequence is present in a concatenated polypeptide comprising each of the sequences set forth in Table 9A, Table 9B, or Table 9C, optionally wherein the concatenated polypeptide comprises the order of sequences set forth in Table 9A, Table 9B, or Table 9C,
at least one MHC class I epitope comprising a polypeptide sequence as set forth in Table A and/or Table C or MHC class II epitope comprising a polypeptide sequence as set forth in Table B, wherein the encoded SARS-CoV-2 immunogenic polypeptide is conserved between SARS-CoV-2 and a Coronavirus species and/or sub-species other than SARS-CoV-2, optionally wherein the Coronavirus species and/or sub-species other than SARS-CoV-2 is Severe acute respiratory syndrome (SARS) and/or Middle East respiratory syndrome (MERS),
one or more validated epitopes and/or at least 4, 5, 6, or 7 predicted epitopes, wherein at least 85%, 90%, or 95% of a population carries at least one HLA validated to present at least one of the one or more validated epitopes and/or at least one HLA predicted to present each of the at least 4, 5, 6, or 7 predicted epitopes,
a SARS-CoV-2 Spike protein or an epitope-containing fragment thereof corresponding to an isolate other than a B.1.1.529 SARS-CoV-2 isolate, optionally comprising a Spike polypeptide sequence as set forth in SEQ ID NO:59, optionally wherein the Spike polypeptide comprises a D614G mutation with reference to SEQ ID NO:59, and optionally wherein the Spike polypeptide is encoded by the nucleotide sequence shown in SEQ ID NO:79, SEQ ID NO:83, SEQ ID NO:85, or SEQ ID NO:87,
a SARS-CoV-2 modified Spike protein comprising a mutation selected from the group consisting of: a Spike R682 mutation, a Spike R683 mutation, a Spike R685 mutation, a Spike R815 mutation, a Spike K986P mutation, a Spike V987P mutation, and combinations thereof with reference to the Spike polypeptide sequence as set forth in SEQ ID NO:59, and optionally wherein the modified Spike protein comprises a polypeptide sequence as set forth in SEQ ID NO:60 or SEQ ID NO:90 or an epitope-containing fragment thereof,
a SARS-CoV-2 Membrane protein comprising a Membrane polypeptide sequence as set forth in SEQ ID NO:61 or an epitope-containing fragment thereof,
a SARS-CoV-2 Nucleocapsid protein comprising a Nucleocapsid polypeptide sequence as set forth in SEQ ID NO:62 or an epitope-containing fragment thereof,
a SARS-CoV-2 Envelope protein comprising an Envelope polypeptide sequence as set forth in SEQ ID NO:63 or an epitope-containing fragment thereof,
a variant of any of the above comprising a mutation found in 1% or greater of SARS-CoV-2 subtypes, optionally wherein the variant comprises a SARS-CoV-2 variant shown in Table 1,
or combinations thereof; and
   wherein the immunogenic polypeptide optionally comprises a N-terminal linker and/or a C-terminal linker; (ii) optionally, a second promoter nucleotide sequence operably linked to at least one of the SARS-CoV-2 derived nucleic acid sequences; and (iii) optionally, at least one MHC class II epitope-encoding nucleic acid sequence; and (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, optionally wherein the exogenous poly(A) sequence comprises an SV40 poly(A) signal sequence or a Bovine Growth Hormone (BGH) poly(A) signal sequence.

Also provided herein is an antigen-based vaccine comprising (i) a SARS-CoV-2 variant Spike protein corresponding to a B.1.1.529 SARS-CoV-2 isolate optionally comprising the Spike polypeptide sequence as set forth in SEQ ID NO: 27974 and/or is encoded by nucleotides 9714-13,526 of the nucleotide sequence set forth in SEQ ID NO: 27976, or a SARS-CoV-2 variant Spike protein corresponding to a B.1.1.529 BA5 SARS-CoV-2 subvariant optionally comprising the Spike polypeptide sequence as set forth in SEQ ID NO: 27978 and/or encoded by SEQ ID NO: 27980, optionally wherein (a) the B.1.1.529 isolate Spike protein or fragment comprises a mutation selected from the group consisting of: a Spike R679 mutation, a Spike R680 mutation, a Spike R682 mutation, a Spike K983P mutation, a Spike V984P mutation, and combinations thereof with reference to the Spike polypeptide sequence as set forth in SEQ ID NO:27977 or (b) the B.1.1.529 BA5 subvariant Spike protein or fragment comprises a mutation selected from the group consisting of: a Spike R677 mutation, a Spike R678 mutation, a Spike R680 mutation, a Spike K981P mutation, a Spike V982P mutation, and combinations thereof with reference to the Spike polypeptide sequence as set forth in SEQ ID NO: 27979, and optionally wherein the antigen-based vaccine further comprises at least one additional SARS-CoV-2 derived immunogenic polypeptide, wherein the additional immunogenic polypeptide comprises:
at least one MHC class I epitope comprising a polypeptide sequence as set forth in Table A,
at least one MHC class II epitope comprising a polypeptide sequence as set forth in Table B,
at least one MHC class I epitope comprising a polypeptide sequence as set forth in Table C, optionally wherein the at least one MHC I epitope is present in a concatenated polypeptide sequence as set forth in SEQ ID NO:57 or SEQ ID NO:58,
at least one polypeptide sequence as set forth in Table 6, or an epitope-containing fragment thereof, optionally wherein the at least one polypeptide sequence is present in a concatenated polypeptide sequence as set forth in SEQ ID NO:92,
at least one polypeptide sequence as set forth in Table 9A, Table 9B, or Table 9C, or an epitope-containing fragment thereof, optionally wherein the at least one polypeptide sequence is present in a concatenated polypeptide comprising each of the sequences set forth in Table 9A, Table 9B, or Table 9C, optionally wherein the concatenated polypeptide comprises the order of sequences set forth in Table 9A, Table 9B, or Table 9C,
at least one MHC class I epitope comprising a polypeptide sequence as set forth in Table A and/or Table C or MHC class II epitope comprising a polypeptide sequence as set forth in Table B, wherein the encoded SARS-CoV-2 immunogenic polypeptide is conserved between SARS-CoV-2 and a Coronavirus species and/or sub-species other than SARS-CoV-2, optionally wherein the Coronavirus species and/or sub-species other than SARS-CoV-2 is Severe acute respiratory syndrome (SARS) and/or Middle East respiratory syndrome (MERS),
one or more validated epitopes and/or at least 4, 5, 6, or 7 predicted epitopes, wherein at least 85%, 90%, or 95% of a population carries at least one HLA validated to present at least one of the one or more validated epitopes and/or at least one HLA predicted to present each of the at least 4, 5, 6, or 7 predicted epitopes,
a SARS-CoV-2 Spike protein comprising a Spike polypeptide sequence as set forth in SEQ ID NO:59 or an epitope-containing fragment thereof, optionally wherein the Spike polypeptide comprises a D614G mutation with reference to SEQ ID NO:59, and optionally wherein the Spike polypeptide is encoded by the nucleotide sequence shown in SEQ ID NO:79, SEQ ID NO:83, SEQ ID NO:85, or SEQ ID NO:87,
a SARS-CoV-2 modified Spike protein comprising a mutation selected from the group consisting of: a Spike R682 mutation, a Spike R683 mutation, a Spike R685 mutation, a Spike R815 mutation, a Spike K986P mutation, a Spike V987P mutation, and combinations thereof with reference to the Spike polypeptide sequence as set forth in SEQ ID NO:59, and optionally wherein the modified Spike protein comprises a polypeptide sequence as set forth in SEQ ID NO:60 or SEQ ID NO:90 or an epitope-containing fragment thereof,
a SARS-CoV-2 Membrane protein comprising a Membrane polypeptide sequence as set forth in SEQ ID NO:61 or an epitope-containing fragment thereof,
a SARS-CoV-2 Nucleocapsid protein comprising a Nucleocapsid polypeptide sequence as set forth in SEQ ID NO:62 or an epitope-containing fragment thereof,
a SARS-CoV-2 Envelope protein comprising an Envelope polypeptide sequence as set forth in SEQ ID NO:63 or an epitope-containing fragment thereof,
a variant of any of the above comprising a mutation found in 1% or greater of SARS-CoV-2 subtypes, optionally wherein the variant comprises a SARS-CoV-2 variant shown in Table 1,
or combinations thereof; and
   wherein the immunogenic peptide optionally comprises a N-terminal linker and/or a C-terminal linker (ii) optionally, at least one MHC class II antigen; and (iii) optionally, at least one GPGPG amino acid linker sequence (SEQ ID NO:56).

Also provided for herein is a composition for delivery of an antigen expression system, comprising: the antigen expression system, wherein the antigen expression system comprises: (a) optionally, one or more vectors, the one or more vectors comprising: a vector backbone, wherein the vector backbone comprises: (i) at least one promoter nucleotide sequence, and (ii) at least one polyadenylation (poly(A)) sequence; and (b) an antigen cassette, optionally wherein the antigen cassette is inserted into the vector backbone when present, and wherein the antigen cassette comprises: (i) three SARS-CoV-2 derived nucleic acid sequences encoding immunogenic polypeptides, wherein the immunogenic polypeptides comprises: (A) a SARS-CoV-2 derived nucleic acid sequence encoding an immunogenic polypeptide, wherein the immunogenic polypeptide comprises a SARS-CoV-2 variant Spike protein corresponding to a B.1.1.529 SARS-CoV-2 isolate optionally comprising the Spike polypeptide sequence as set forth in SEQ ID NO: 27974 and/or is encoded by nucleotides 9714-13,526 of the nucleotide sequence set forth in SEQ ID NO: 27976, or a SARS-CoV-2 variant Spike protein corresponding to a B.1.1.529 BA5 SARS-CoV-2 subvariant optionally comprising the Spike polypeptide sequence as set forth in SEQ ID NO: 27978 and/or encoded by SEQ ID NO: 27980, optionally wherein (a) the B.1.1.529 isolate Spike protein or fragment comprises a mutation selected from the group consisting of: a Spike R679 mutation, a Spike R680 mutation, a Spike R682 mutation, a Spike K983P mutation, a Spike V984P mutation, and combinations thereof with reference to the Spike polypeptide sequence as set forth in SEQ ID NO:27977 or (b) the B.1.1.529 BA5 subvariant Spike protein or fragment comprises a mutation selected from the group consisting of: a Spike R677 mutation, a Spike R678 mutation, a Spike R680 mutation, a Spike K981P mutation, a Spike V982P mutation, and combinations thereof with reference to the Spike polypeptide sequence as set forth in SEQ ID NO: 27979; (B) at least one polypeptide sequence as set forth in Table 9C, or an epitope-containing fragment thereof, optionally wherein the at least one polypeptide sequence is present in a concatenated polypeptide comprising each of the sequences set forth in Table 9C, optionally wherein the concatenated polypeptide comprises the order of sequences set forth in Table 9C, and (C) a SARS-CoV-2 Nucleocapsid protein, optionally comprising a Nucleocapsid polypeptide sequence as set forth in SEQ ID NO:62 or an epitope-containing fragment thereof, wherein each of the SAR-CoV-2 SARS-CoV-2 derived nucleic acid sequences comprises; (I) optionally, a 5′ linker sequence, and (II) optionally, a 3′ linker sequence; (ii) optionally, a second promoter nucleotide sequence operably linked to one or more of the three SARS-CoV-2 derived nucleic acid sequences; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone optionally wherein the exogenous poly(A) sequence comprises an SV40 poly(A) signal sequence or a Bovine Growth Hormone (BGH) poly(A) signal sequence.

Also provided for herein is a composition for delivery of an antigen expression system, comprising: the antigen expression system, wherein the antigen expression system comprises: (a) one or more vectors, the one or more vectors comprising: a vector backbone, wherein the vector backbone comprises a chimpanzee adenovirus vector, optionally wherein the chimpanzee adenovirus vector is a ChAdV68 vector, or an alphavirus vector, optionally wherein the alphavirus vector is a Venezuelan equine encephalitis virus vector, and wherein the vector backbone comprises: (i) at least one promoter nucleotide sequence, and (ii) at least one polyadenylation (poly(A)) sequence; and (b) an antigen cassette, wherein the antigen cassette is inserted into the vector backbone such that the antigen cassette is operably linked to the at least one promoter nucleotide sequence, and wherein the antigen cassette comprises: (i) three SARS-CoV-2 derived nucleic acid sequences encoding immunogenic polypeptides, wherein the immunogenic polypeptides comprises: (A) a SARS-CoV-2 variant Spike protein corresponding to a B.1.1.529 SARS-CoV-2 isolate optionally comprising the Spike polypeptide sequence as set forth in SEQ ID NO: 27974 and/or is encoded by nucleotides 9714-13,526 of the nucleotide sequence set forth in SEQ ID NO: 27976, or a SARS-CoV-2 variant Spike protein corresponding to a B.1.1.529 BA5 SARS-CoV-2 subvariant optionally comprising the Spike polypeptide sequence as set forth in SEQ ID NO: 27978 and/or encoded by SEQ ID NO: 27980, optionally wherein (a) the B.1.1.529 isolate Spike protein or fragment comprises a mutation selected from the group consisting of: a Spike R679 mutation, a Spike R680 mutation, a Spike R682 mutation, a Spike K983P mutation, a Spike V984P mutation, and combinations thereof with reference to the Spike polypeptide sequence as set forth in SEQ ID NO:27977 or (b) the B.1.1.529 BA5 subvariant Spike protein or fragment comprises a mutation selected from the group consisting of: a Spike R677 mutation, a Spike R678 mutation, a Spike R680 mutation, a Spike K981P mutation, a Spike V982P mutation, and combinations thereof with reference to the Spike polypeptide sequence as set forth in SEQ ID NO: 27979, and (B) at least one polypeptide sequence as set forth in Table 9C, or an epitope-containing fragment thereof, optionally wherein the at least one polypeptide sequence is present in a concatenated polypeptide comprising each of the sequences set forth in Table 9C, optionally wherein the concatenated polypeptide comprises the order of sequences set forth in Table 9C, and (C) a SARS-CoV-2 Nucleocapsid protein, optionally comprising a Nucleocapsid polypeptide sequence as set forth in SEQ ID NO:62 or an epitope-containing fragment thereof, (ii) optionally, a second promoter nucleotide sequence operably linked to at least one of the three SARS-CoV-2 derived nucleic acid sequences; and (iii) optionally, at least one MHC class II epitope-encoding nucleic acid sequence; (iv) optionally, at least one nucleic acid sequence encoding a GPGPG amino acid linker sequences (SEQ ID NO:56); and (v) optionally, at least one second poly(A) sequence, wherein the second poly(A) sequence is a native poly(A) sequence or an exogenous poly(A) sequence to the vector backbone optionally wherein the exogenous poly(A) sequence comprises an SV40 poly(A) signal sequence or a Bovine Growth Hormone (BGH) poly(A) signal sequence.

Also provided for herein is a composition for delivery of an antigen expression system, comprising: the antigen expression system, wherein the antigen expression system comprises: (a) a vector backbone, wherein the vector backbone comprises an alphavirus vector, wherein the alphavirus vector is a Venezuelan equine encephalitis virus vector, and wherein the vector backbone comprises: (i) a subgenomic promoter nucleotide sequence, and (ii) at least one polyadenylation (poly(A)) sequence; and (b) an antigen cassette, wherein the antigen cassette is inserted into the vector backbone such that the antigen cassette is operably linked to the at least one promoter nucleotide sequence, and wherein the antigen cassette comprises: (i) three SARS-CoV-2 derived nucleic acid sequences encoding immunogenic polypeptides, wherein the immunogenic polypeptides comprises: (A) a SARS-CoV-2 derived nucleic acid sequence encoding an immunogenic polypeptide, wherein the immunogenic polypeptide comprises a SARS-CoV-2 variant Spike protein corresponding to a B.1.1.529 SARS-CoV-2 isolate optionally comprising the Spike polypeptide sequence as set forth in SEQ ID NO: 27974 and/or is encoded by nucleotides 9714-13,526 of the nucleotide sequence set forth in SEQ ID NO: 27976, or a SARS-CoV-2 variant Spike protein corresponding to a B.1.1.529 BA5 SARS-CoV-2 subvariant optionally comprising the Spike polypeptide sequence as set forth in SEQ ID NO: 27978 and/or encoded by SEQ ID NO: 27980, optionally wherein (a) the B.1.1.529 isolate Spike protein or fragment comprises a mutation selected from the group consisting of: a Spike R679 mutation, a Spike R680 mutation, a Spike R682 mutation, a Spike K983P mutation, a Spike V984P mutation, and combinations thereof with reference to the Spike polypeptide sequence as set forth in SEQ ID NO:27977 or (b) the B.1.1.529 BA5 subvariant Spike protein or fragment comprises a mutation selected from the group consisting of: a Spike R677 mutation, a Spike R678 mutation, a Spike R680 mutation, a Spike K981P mutation, a Spike V982P mutation, and combinations thereof with reference to the Spike polypeptide sequence as set forth in SEQ ID NO: 27979; (B) at least one polypeptide sequence as set forth in Table 9C, or an epitope-containing fragment thereof, optionally wherein the at least one polypeptide sequence is present in a concatenated polypeptide comprising each of the sequences set forth in Table 9C, optionally wherein the concatenated polypeptide comprises the order of sequences set forth in Table 9C, and (C) a SARS-CoV-2 Nucleocapsid protein, optionally comprising a Nucleocapsid polypeptide sequence as set forth in SEQ ID NO:62 or an epitope-containing fragment thereof; (ii) a second subgenomic promoter nucleotide sequence operably linked to at least one of the three SARS-CoV-2 derived nucleic acid sequences; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone optionally wherein the exogenous poly(A) sequence comprises an SV40 poly(A) signal sequence or a Bovine Growth Hormone (BGH) poly(A) signal sequence.

Also provided for herein is a composition for delivery of an antigen expression system, wherein the antigen expression system comprises the nucleotide sequence as set forth in SEQ ID NO:27976, optionally wherein the nucleotides encoding a SARS-CoV-2 variant Spike protein corresponding to the B.1.1.529 SARS-CoV-2 isolate are substituted with the nucleotides encoding a SARS-CoV-2 variant Spike protein corresponding to a B.1.1.529 BA5 SARS-CoV-2 subvariant.

Also provided for herein is a composition for delivery of an antigen expression system, wherein the antigen expression system comprises the nucleotide sequence as set forth in nucleotides 7,571 to 13,526 of SEQ ID NO:27976, optionally wherein the nucleotides encoding a SARS-CoV-2 variant Spike protein corresponding to the B.1.1.529 SARS-CoV-2 isolate are substituted with the nucleotides encoding a SARS-CoV-2 variant Spike protein corresponding to a B.1.1.529 BA5 SARS-CoV-2 subvariant.

Also provided for herein is a composition for delivery of an antigen expression system, comprising: the antigen expression system, wherein the antigen expression system comprises: (a) a vector backbone, wherein the vector backbone comprises an alphavirus vector, optionally wherein the alphavirus vector is a Venezuelan equine encephalitis virus vector, and wherein the vector backbone comprises: (i) a subgenomic promoter nucleotide sequence, and (ii) a polyadenylation (poly(A)) sequence; and (b) an antigen cassette, wherein the antigen cassette is inserted into the vector backbone such that the antigen cassette is operably linked to the subgenomic promoter nucleotide sequence, and wherein the antigen cassette comprises, in order from 5′ to 3′: (i) a nucleotide sequence encoding a SARS-CoV-2 Nucleocapsid protein comprising a Nucleocapsid polypeptide sequence as set forth in SEQ ID NO:62; (ii) a 2A ribosome skipping sequence element; (iii) a nucleotide sequence encoding a concatenated polypeptide comprising each of the sequences set forth in Table 9C and in the order of sequences set forth in Table 9C; (iv) a nucleotide sequence encoding at least one universal MHC class II epitope, optionally further encoding one or more GPGPG amino acid linker sequences (SEQ ID NO:56) at the 5′ terminus, 3′ terminus, and/or in between concatenated universal MHC class II epitope sequences; (v) a second subgenomic promoter nucleotide sequence; and (vi) a nucleotide sequence encoding a B.1.1.529 SARS-CoV-2 isolate optionally comprising the Spike polypeptide sequence as set forth in SEQ ID NO: 27974 and/or is encoded by nucleotides 9714-13,526 of the nucleotide sequence set forth in SEQ ID NO: 27976, or a SARS-CoV-2 variant Spike protein corresponding to a B.1.1.529 BA5 SARS-CoV-2 subvariant optionally comprising the Spike polypeptide sequence as set forth in SEQ ID NO: 27978 and/or encoded by SEQ ID NO: 27980.

Also provided for herein is a composition for delivery of an antigen expression system, comprising a self-amplifying alphavirus-based expression system comprising a vector selected from the group of sequences consisting of: SEQ ID NO: 27983, SEQ ID NO:27981, SEQ ID NO:27982, SEQ ID NO: 27976, and SEQ ID NO: 27976 with Spike encoding sequences substituted with the sequence set forth in SEQ ID NO:27980.

In some aspects, the composition for delivery of the self-amplifying alphavirus-based expression system comprises at least 3 μg of each of the one or more vectors. In some aspects, the composition for delivery of the self-amplifying alphavirus-based expression system comprises at least 10 μg of each of the one or more vectors. In some aspects, the composition for delivery of the self-amplifying alphavirus-based expression system comprises at least 30 μg of each of the one or more vectors. In some aspects, the composition for delivery of the self-amplifying alphavirus-based expression system comprises at least 100 μg of each of the one or more vectors. In some aspects, the composition for delivery of the self-amplifying alphavirus-based expression system comprises at least 300 μg of each of the one or more vectors. In some aspects, the composition for delivery of the self-amplifying alphavirus-based expression system comprises at least 400 μg, at least 500 μg, at least 600 μg, at least 700 μg, at least 800 μg, at least 900 μg, at least 1000 μg of each of the one or more vectors. In some aspects, the composition for delivery of the self-amplifying alphavirus-based expression system comprises between 10-30 μg, 10-100 μg, 10-300 μg, 30-100 μg, 30-300 μg, or 100-300 μg of each of the one or more vectors. In some aspects, the composition for delivery of the self-amplifying alphavirus-based expression system comprises between 3-10 μg, 3-30 μg, 3-100 μg, 3-300 μg, 10-30 μg, or 100-300 μg of each of the one or more vectors. In some aspects, the composition for delivery of the self-amplifying alphavirus-based expression system comprises between 10-500 μg, 10-1000 μg, 30-500 μg, 30-1000 μg, or 500-1000 μg of each of the one or more vectors. In some aspects, the composition for delivery of the self-amplifying alphavirus-based expression system comprises 10 μg, 30 μg, 100 μg, or 300 μg of each of the one or more vectors. In some aspects, the composition for delivery of the self-amplifying alphavirus-based expression system comprises 400 μg, 500 μg, 600 μg, 700 μg, 800 μg, 900 μg, or 1000 μg of each of the one or more vectors. In some aspects, the composition for delivery of the self-amplifying alphavirus-based expression system comprises less than or equal to 300 μg of each of the one or more vectors. In some aspects, the composition for delivery of the self-amplifying alphavirus-based expression system comprises less than or equal to 100 μg of each of the one or more vectors. In some aspects, the composition for delivery of the self-amplifying alphavirus-based expression system comprises less than or equal to 30 μg of each of the one or more vectors. A dose of can represent the total content of RNA/samRNA administered. A dose of can represent the total content of RNA/samRNA administered and include only a single distinct samRNA construct.

In some aspects, an ordered sequence of one or more of the SARS-CoV-2 derived nucleic acid sequences encoding the immunogenic polypeptide is described in the formula, from 5′ to 3′, comprising:

Pa-(L5b-Nc-L3d)X-(G5e-Uf)Y-G3g

wherein P comprises the second promoter nucleotide sequence, where a=0 or 1, N comprises one of the SARS-CoV-2 derived nucleic acid sequences, where c=1, optionally wherein each N encodes a polypeptide sequence as set forth in Table A, Table B, and/or Table C, L5 comprises the 5′ linker sequence, where b=0 or 1, L3 comprises the 3′ linker sequence, where d=0 or 1, G5 comprises one of the at least one nucleic acid sequences encoding a GPGPG amino acid linker (SEQ ID NO: 56), where e=0 or 1, G3 comprises one of the at least one nucleic acid sequences encoding a GPGPG amino acid linker (SEQ ID NO: 56), where g=0 or 1, U comprises one of the at least one MHC class II epitope-encoding nucleic acid sequence, where f=1, X=1 to 400, where for each X the corresponding Nc is a SARS-CoV-2 derived nucleic acid sequence, and Y=0, 1, or 2, where for each Y the corresponding Uf is a universal MHC class II epitope-encoding nucleic acid sequence, optionally wherein the at least one universal sequence comprises at least one of Tetanus toxoid and PADRE, or a MHC class II SARS-CoV-2 derived epitope-encoding nucleic acid sequence.

In some aspects, for each X the corresponding Nc is a distinct SARS-CoV-2 derived nucleic acid sequence. In some aspects, for each Y the corresponding Uf is a distinct MHC class II SARS-CoV-2 derived nucleic acid sequence. In some aspects, b=1, d=1, e=1, g=1, h=1, X=18, Y=2, (i) the vector backbone comprises a ChAdV68 vector, a=1, P is a CMV promoter, the at least one second poly(A) sequence is present, wherein the second poly(A) sequence is an exogenous poly(A) sequence to the vector backbone, and optionally wherein the exogenous poly(A) sequence comprises an SV40 poly(A) signal sequence or a BGH poly(A) signal sequence, or (ii) the vector backbone comprises a Venezuelan equine encephalitis virus vector, a=0, and the antigen cassette is operably linked to an endogenous 26S promoter, and the at least one polyadenylation poly(A) sequence is a poly(A) sequence of at least 80 consecutive A nucleotides (SEQ ID NO: 27940) provided by the backbone, each N encodes a MHC class I epitope 7-15 amino acids in length, a MHC class II epitope, an epitope capable of stimulating a B cell response, or combinations thereof, L5 is a native 5′ linker sequence that encodes a native N-terminal amino acid sequence of the epitope, and wherein the 5′ linker sequence encodes a peptide that is at least 3 amino acids in length, L3 is a native 3′ linker sequence that encodes a native C-terminal amino acid sequence of the epitope, and wherein the 3′ linker sequence encodes a peptide that is at least 3 amino acids in length, and U is each of a PADRE class II sequence and a Tetanus toxoid MHC class II sequence.

In some aspects, the composition further comprises a nanoparticulate delivery vehicle. In some aspects, the nanoparticulate delivery vehicle is a lipid nanoparticle (LNP). In some aspects, the LNP comprises ionizable amino lipids. In some aspects, the ionizable amino lipids comprise MC3-like (dilinoleylmethyl-4-dimethylaminobutyrate) molecules. In some aspects, the nanoparticulate delivery vehicle encapsulates the antigen expression system.

In some aspects, the antigen cassette is integrated between the at least one promoter nucleotide sequence and the at least one poly(A) sequence. In some aspects, the at least one promoter nucleotide sequence is operably linked to the SARS-CoV-2 derived nucleic acid sequence.

In some aspects, the one or more vectors comprise one or more +-stranded RNA In some aspects, the one or more +-stranded RNA vectors comprise a 5′ 7-methylguanosine (m7g) cap. In some aspects, the one or more +-stranded RNA vectors are produced by in vitro transcription. In some aspects, the one or more vectors are self-replicating within a mammalian cell.

In some aspects, the backbone comprises at least one nucleotide sequence of an Aura virus, a Fort Morgan virus, a Venezuelan equine encephalitis virus, a Ross River virus, a Semliki Forest virus, a Sindbis virus, or a Mayaro virus. In some aspects, the backbone comprises at least one nucleotide sequence of a Venezuelan equine encephalitis virus. In some aspects, the backbone comprises at least sequences for nonstructural protein-mediated amplification, a 26S promoter sequence, a poly(A) sequence, a nonstructural protein 1 (nsP1) gene, a nsP2 gene, a nsP3 gene, and a nsP4 gene encoded by the nucleotide sequence of the Aura virus, the Fort Morgan virus, the Venezuelan equine encephalitis virus, the Ross River virus, the Semliki Forest virus, the Sindbis virus, or the Mayaro virus. In some aspects, the backbone comprises at least sequences for nonstructural protein-mediated amplification, a 26S promoter sequence, and a poly(A) sequence encoded by the nucleotide sequence of the Aura virus, the Fort Morgan virus, the Venezuelan equine encephalitis virus, the Ross River virus, the Semliki Forest virus, the Sindbis virus, or the Mayaro virus. In some aspects, sequences for nonstructural protein-mediated amplification are selected from the group consisting of: an alphavirus 5′ UTR, a 51-nt CSE, a 24-nt CSE, a 26S subgenomic promoter sequence, a 19-nt CSE, an alphavirus 3′ UTR, or combinations thereof. In some aspects, the backbone does not encode structural virion proteins capsid, E2 and E1. In some aspects, the antigen cassette is inserted in place of structural virion proteins within the nucleotide sequence of the Aura virus, the Fort Morgan virus, the Venezuelan equine encephalitis virus, the Ross River virus, the Semliki Forest virus, the Sindbis virus, or the Mayaro virus. In some aspects, the Venezuelan equine encephalitis virus comprises the sequence of SEQ ID NO:3 or SEQ ID NO:5. In some aspects, the Venezuelan equine encephalitis virus comprises the sequence of SEQ ID NO:3 or SEQ ID NO:5 further comprising a deletion between base pair 7544 and 11176. In some aspects, the backbone comprises the sequence set forth in SEQ ID NO:6 or SEQ ID NO:7. In some aspects, the antigen cassette is inserted at position 7544 to replace the deletion between base pairs 7544 and 11176 as set forth in the sequence of SEQ ID NO:3 or SEQ ID NO:5. In some aspects, the insertion of the antigen cassette provides for transcription of a polycistronic RNA comprising the nsP1-4 genes and the at least one SARS-CoV-2 derived nucleic acid sequence, wherein the nsP1-4 genes and the at least one SARS-CoV-2 derived nucleic acid sequence are in separate open reading frames. In some aspects, the at least one promoter nucleotide sequence is the native 26S promoter nucleotide sequence encoded by the backbone.

In some aspects, the backbone comprises at least one nucleotide sequence of a chimpanzee adenovirus vector, optionally wherein the chimpanzee adenovirus vector is a ChAdV68 vector. In some aspects, the ChAdV68 vector backbone comprises the sequence set forth in SEQ ID NO:1. In some aspects, the ChAdV68 vector backbone comprises the sequence set forth in SEQ ID NO:1, except that the sequence is fully deleted or functionally deleted in at least one gene selected from the group consisting of the chimpanzee adenovirus E1A, E1B, E2A, E2B, E3, E4, L1, L2, L3, L4, and L5 genes of the sequence set forth in SEQ ID NO:1, optionally wherein the sequence is fully deleted or functionally deleted in: (1) E1A and E1B; (2) E1A, E1B, and E3; or (3) E1A, E1B, E3, and E4 of the sequence set forth in SEQ ID NO:1. In some aspects, the ChAdV68 vector backbone comprises a gene or regulatory sequence obtained from the sequence of SEQ ID NO:1, optionally wherein the gene is selected from the group consisting of the chimpanzee adenovirus inverted terminal repeat (ITR), E1A, E1B, E2A, E2B, E3, E4, L1, L2, L3, L4, and L5 genes of the sequence set forth in SEQ ID NO:1. In some aspects, the ChAdV68 vector backbone comprises a partially deleted E4 gene comprising a deleted or partially-deleted E4orf2 region and a deleted or partially-deleted E4orf3 region, and optionally a deleted or partially-deleted E4orf4 region. In some aspects, the ChAdV68 vector backbone comprises at least nucleotides 2 to 36,518 of the sequence set forth in SEQ ID NO:1 and further comprising: (1) an E1 deletion of at least nucleotides 577 to 3403 of the sequence shown in SEQ ID NO:1, (2) an E3 deletion of at least nucleotides 27,125 to 31,825 of the sequence shown in SEQ ID NO:1, and (3) an E4 deletion of at least nucleotides 34,916 to 35,642 of the sequence shown in SEQ ID NO:1; optionally wherein the antigen cassette is inserted within the E1 deletion. In some aspects, the ChAdV68 vector backbone comprises the sequence set forth in SEQ ID NO:75, optionally wherein the antigen cassette is inserted within the E1 deletion. In some aspects, the ChAdV68 vector backbone comprises one or more deletions between base pair number 577 and 3403 or between base pair 456 and 3014, and optionally wherein the vector further comprises one or more deletions between base pair 27,125 and 31,825 or between base pair 27,816 and 31,333 of the sequence set forth in SEQ ID NO:1. In some aspects, the ChAdV68 vector backbone comprises one or more deletions between base pair number 3957 and 10346, base pair number 21787 and 23370, and base pair number 33486 and 36193 of the sequence set forth in SEQ ID NO:1. In some aspects, the wherein the cassette is inserted in the ChAdV backbone at the E1 region, E3 region, and/or any deleted AdV region that allows incorporation of the cassette. In some aspects, the ChAdV backbone is generated from one of a first generation, a second generation, or a helper-dependent adenoviral vector

In some aspects, the at least one promoter nucleotide sequence is selected from the group consisting of: a CMV, a SV40, an EF-1, a RSV, a PGK, a HSA, a MCK, and a EBV promoter sequence. In some aspects, the at least one promoter nucleotide sequence is a CMV promoter sequence. In some aspects, the at least one promoter nucleotide sequence is an exogenous RNA promoter. In some aspects, the second promoter nucleotide sequence is a 26S promoter nucleotide sequence or a CMV promoter nucleotide sequence. In some aspects, the second promoter nucleotide sequence comprises multiple 26S promoter nucleotide sequences or multiple CMV promoter nucleotide sequences, wherein each 26S promoter nucleotide sequence or CMV promoter nucleotide sequence provides for transcription of one or more of the separate open reading frames.

In some aspects, one or more of the cassettes are at least 100, 200, 300, 400, 500, 600, 700, 800, or 900 nucleotides in length. In some aspects, one or more of the cassettes are at least 1000, 2000, 3000, 4000, 5000, 6000, 7000, 8000, 9000, or 10000 nucleotides in length. In some aspects, the one or more vectors are capable of driving expression of a cassette that is at least 3500 nucleotides in length. In some aspects, the one or more vectors are capable of driving expression of a cassette that is at least 6000 nucleotides in length.

In some aspects, at least one of the at least one SARS-CoV-2 derived nucleic acid sequences encodes a polypeptide sequence or portion thereof that is presented by MHC class I. In some aspects, at least one of the at least one SARS-CoV-2 derived nucleic acid sequences encodes a polypeptide sequence or portion thereof that is presented by MHC class II. In some aspects, at least one of the at least one SARS-CoV-2 derived nucleic acid sequences encodes a polypeptide sequence or portion thereof capable of stimulating a B cell response, optionally wherein the polypeptide sequence or portion thereof capable of stimulating a B cell response comprises a full-length protein, a protein domain, a protein subunit, or an antigenic fragment predicted or known to be capable of being bound by an antibody.

In some aspects, each SARS-CoV-2 derived nucleic acid sequence is linked directly to one another. In some aspects, at least one of the at least one SARS-CoV-2 derived nucleic acid sequences is linked to a distinct SARS-CoV-2 derived nucleic acid sequence with a nucleic acid sequence encoding a linker. In some aspects, the linker links In some aspects, the linker is selected from the group consisting of: (1) consecutive glycine residues, at least 2, 3, 4, 5, 6, 7, 8, 9, or 10 residues in length (SEQ ID NO: 27941); (2) consecutive alanine residues, at least 2, 3, 4, 5, 6, 7, 8, 9, or 10 residues in length (SEQ ID NO: 27942); (3) two arginine residues (RR); (4) alanine, alanine, tyrosine (AAY); (5) a consensus sequence at least 2, 3, 4, 5, 6, 7, 8, 9, or 10 amino acid residues in length that is processed efficiently by a mammalian proteasome; (6) one or more native sequences flanking the antigen derived from the cognate protein of origin and that is at least 2, 3, 4, 5, 6, 7, 8, 9, 10, 11, 12, 13, 14, 15, 16, 17, 18, 19, 20, or 2-20 amino acid residues in length; and (7) a furin or TEV cleavage sequence. In some aspects, the linker links two MHC class II sequences or an MHC class II sequence to an MHC class I sequence. In some aspects, the linker comprises the sequence GPGPG (SEQ ID NO: 56).

In some aspects, at least one sequence of the at least one SARS-CoV-2 derived nucleic acid sequences is linked, operably or directly, to a separate or contiguous sequence that enhances the expression, stability, cell trafficking, processing and presentation, and/or immunogenicity of the at least one SARS-CoV-2 derived nucleic acid sequences. In some aspects, the separate or contiguous sequence comprises at least one of: a ubiquitin sequence, a ubiquitin sequence modified to increase proteasome targeting (e.g., the ubiquitin sequence contains a Gly to Ala substitution at position 76), an immunoglobulin signal sequence (e.g., IgK), a major histocompatibility class I sequence, lysosomal-associated membrane protein (LAMP)-1, human dendritic cell lysosomal-associated membrane protein, and a major histocompatibility class II sequence; optionally wherein the ubiquitin sequence modified to increase proteasome targeting is A76.

In some aspects, at least one of the at least one SARS-CoV-2 derived nucleic acid sequences encodes two or more distinct polypeptides predicted or validated to be capable of presentation by at least one HLA allele.

In some aspects, each of the at least one SARS-CoV-2 derived nucleic acid sequences encodes a polypeptide sequence or portion thereof that is less than 50%, less than 49%, less than 48%, less than 47%, less than 46%, less than 45%, less than 45%, less than 43%, less than 42%, less than 41%, less than 40%, less than 39%, less than 38%, less than 37%, less than 36%, less than 35%, less than 34%, or less than 33% of the translated, corresponding full-length SARS-CoV-2 protein.

In some aspects, each of the at least one SARS-CoV-2 derived nucleic acid sequences encodes a polypeptide sequence or portion thereof that does not encode a functional protein, functional protein domain, functional protein subunit, or functional protein fragment of the translated, corresponding SARS-CoV-2 protein.

In some aspects, two or more of the at least one SARS-CoV-2 derived nucleic acid sequences are derived from the same SARS-CoV-2 gene. In some aspects, the two or more SARS-CoV-2 derived nucleic acid sequences derived from the same SARS-CoV-2 gene are ordered such that a first nucleic acid sequence cannot be immediately followed by or linked to a second nucleic acid sequence if the second nucleic acid sequence follows first nucleic acid sequence in the corresponding SARS-CoV-2 gene.

In some aspects, the at least one SARS-CoV-2 derived nucleic acid sequence comprises at least 2-10, 2, 3, 4, 5, 6, 7, 8, 9, or 10 nucleic acid sequences. In some aspects, the at least one SARS-CoV-2 derived nucleic acid sequence comprises at least 11-20, 15-20, 11-100, 11-200, 11-300, 11-400, 11, 12, 13, 14, 15, 16, 17, 18, 19, 20 or up to 400 nucleic acid sequences. In some aspects, the at least one SARS-CoV-2 derived nucleic acid sequence comprises at least 2-400 nucleic acid sequences and wherein at least two of the SARS-CoV-2 derived nucleic acid sequences encode polypeptide sequences or portions thereof that are (1) presented by MHC class I, (2) presented by MHC class II, and/or (3) capable of stimulating a B cell response. In some aspects, at least two of the SARS-CoV-2 derived nucleic acid sequences encode polypeptide sequences or portions thereof that are (1) presented by MHC class I, (2) presented by MHC class II, and/or (3) capable of stimulating a B cell response class.

In some aspects, when administered to the subject and translated, at least one of the antigens encoded by the at least one SARS-CoV-2 derived nucleic acid sequence are presented on antigen presenting cells resulting in an immune response targeting at least one of the antigens on a SARS-CoV-2 infected cell surface. In some aspects, when administered to the subject and translated, at least one of the antigens encoded by the at least one SARS-CoV-2 derived nucleic acid sequence results in an antibody response targeting at least one of the antigens on a SARS-CoV-2 virus. In some aspects, the at least one SARS-CoV-2 derived nucleic acid sequences when administered to the subject and translated, at least one of the MHC class I or class II antigens are presented on antigen presenting cells resulting in an immune response targeting at least one of the antigens on a SARS-CoV-2 infected cell surface, and optionally wherein the expression of each of the at least one SARS-CoV-2 derived nucleic acid sequences is driven by the at least one promoter nucleotide sequence.

In some aspects, each MHC class I epitope-encoding SARS-CoV-2 derived nucleic acid sequence encodes a polypeptide sequence between 8 and 35 amino acids in length, optionally 9-17, 9-25, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids in length. In some aspects, the at least one MHC class II epitope-encoding nucleic acid sequence is present. In some aspects, the at least one MHC class II epitope-encoding nucleic acid sequence is present and comprises at least one MHC class II SARS-CoV-2 derived nucleic acid sequence. In some aspects, the at least one MHC class II epitope-encoding nucleic acid sequence is 12-20, 12, 13, 14, 15, 16, 17, 18, 19, 20, or 20-40 amino acids in length. In some aspects, the at least one MHC class II epitope-encoding nucleic acid sequence is present and comprises at least one universal MHC class II epitope-encoding nucleic acid sequence, optionally wherein the at least one universal sequence comprises at least one of Tetanus toxoid and PADRE, and/or at least one MHC class II SARS-CoV-2 derived epitope-encoding nucleic acid sequence.

In some aspects, the at least one promoter nucleotide sequence or the second promoter nucleotide sequence is inducible. In some aspects, the at least one promoter nucleotide sequence or the second promoter nucleotide sequence is non-inducible. In some aspects, the at least one poly(A) sequence comprises a poly(A) sequence native to the backbone. In some aspects, the at least one poly(A) sequence comprises a poly(A) sequence exogenous to the backbone.

In some aspects, the at least one poly(A) sequence is operably linked to at least one of the at least one SARS-CoV-2 derived nucleic acid sequences. In some aspects, the at least one poly(A) sequence is at least 20, at least 30, at least 40, at least 50, at least 60, at least 70, at least 80, or at least 90 consecutive A nucleotides (SEQ ID NO: 27943). In some aspects, the at least one poly(A) sequence is at least 80 consecutive A nucleotides (SEQ ID NO: 27940). In some aspects, the at least one second poly(A) sequence is present. In some aspects, the at least one second poly(A) sequence comprises an SV40 poly(A) signal sequence or a Bovine Growth Hormone (BGH) poly(A) signal sequence, or a combination of two more SV40 poly(A) signal sequences or BGH poly(A) signal sequence. In some aspects, the at least one second poly(A) sequence comprises two or more second poly(A) sequences, optionally wherein the two or more second poly(A) sequences comprises two or more SV40 poly(A) signal sequences two or more BGH poly(A) signal sequences, or a combination of SV40 poly(A) signal sequences and BGH poly(A) signal sequences.

In some aspects, the antigen cassette further comprises at least one of: an intron sequence, an exogenous intron sequence, a Constitutive Transport Element (CTE), a RNA Transport Element (RTE), a woodchuck hepatitis virus posttranscriptional regulatory element (WPRE) sequence, an internal ribosome entry sequence (IRES) sequence, a nucleotide sequence encoding a 2A self cleaving peptide sequence, a nucleotide sequence encoding a Furin cleavage site, or a sequence in the 5′ or 3′ non-coding region known to enhance the nuclear export, stability, or translation efficiency of mRNA that is operably linked to at least one of the at least one SARS-CoV-2 derived nucleic acid sequences.

In some aspects, the antigen cassette further comprises a reporter gene, including but not limited to, green fluorescent protein (GFP), a GFP variant, secreted alkaline phosphatase, luciferase, a luciferase variant, or a detectable peptide or epitope. In some aspects, the detectable peptide or epitope is selected from the group consisting of an HA tag, a Flag tag, a His-tag, or a V5 tag.

In some aspects, the one or more vectors further comprises one or more nucleic acid sequences encoding at least one immune modulator. In some aspects, the immune modulator is an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof. In some aspects, the antibody or antigen-binding fragment thereof is a Fab fragment, a Fab′ fragment, a single chain Fv (scFv), a single domain antibody (sdAb) either as single specific or multiple specificities linked together (e.g., camelid antibody domains), or full-length single-chain antibody (e.g., full-length IgG with heavy and light chains linked by a flexible linker). In some aspects, the heavy and light chain sequences of the antibody are a contiguous sequence separated by either a self-cleaving sequence such as 2A or IRES; or the heavy and light chain sequences of the antibody are linked by a flexible linker such as consecutive glycine residues. In some aspects, the immune modulator is a cytokine. In some aspects, the cytokine is at least one of IL-2, IL-7, IL-12, IL-15, or IL-21 or variants thereof of each.

In some aspects, a MHC class I or MHC class II epitope-encoding SARS-CoV-2 derived nucleic acid sequence is selected by performing the steps of: (a) obtaining at least one of exome, transcriptome, or whole genome SARS-CoV-2 nucleotide sequencing data from a SARS-CoV-2 virus or SARS-CoV-2 infected cell, wherein the SARS-CoV-2 nucleotide sequencing data is used to obtain data representing peptide sequences of each of a set of antigens; (b) inputting the peptide sequence of each antigen into a presentation model to generate a set of numerical likelihoods that each of the antigens is presented by one or more of the MHC alleles on a SARS-CoV-2 infected cell surface, the set of numerical likelihoods having been identified at least based on received mass spectrometry data; and (c) selecting a subset of the set of antigens based on the set of numerical likelihoods to generate a set of selected antigens which are used to generate the MHC class I or MHC class II epitope-encoding SARS-CoV-2 derived nucleic acid sequence.

In some aspects, each MHC class I or MHC class II epitope-encoding SARS-CoV-2 derived nucleic acid sequences is selected by performing the steps of: (a) obtaining at least one of exome, transcriptome, or whole genome SARS-CoV-2 nucleotide sequencing data from a SARS-CoV-2 virus or SARS-CoV-2 infected cell, wherein the SARS-CoV-2 nucleotide sequencing data is used to obtain data representing peptide sequences of each of a set of antigens; (b) inputting the peptide sequence of each antigen into a presentation model to generate a set of numerical likelihoods that each of the antigens is presented by one or more of the MHC alleles on a SARS-CoV-2 infected cell surface, the set of numerical likelihoods having been identified at least based on received mass spectrometry data; and (c) selecting a subset of the set of antigens based on the set of numerical likelihoods to generate a set of selected antigens which are used to generate the at least 18 SARS-CoV-2 derived nucleic acid sequences. In some aspects, a number of the set of selected antigens is 2-20. In some aspects, the presentation model represents dependence between: (a) presence of a pair of a particular one of the MHC alleles and a particular amino acid at a particular position of a peptide sequence; and (b) likelihood of presentation on a SARS-CoV-2 infected cell surface, by the particular one of the MHC alleles of the pair, of such a peptide sequence comprising the particular amino acid at the particular position. In some aspects, selecting the set of selected antigens comprises selecting antigens that have an increased likelihood of being presented on a SARS-CoV-2 infected cell surface relative to unselected antigens based on the presentation model, optionally wherein the selected antigens have been validated as being presented by one or more specific HLA alleles. In some aspects, selecting the set of selected antigens comprises selecting antigens that have an increased likelihood of being capable of inducing a SARS-CoV-2 specific immune response in the subject relative to unselected antigens based on the presentation model. In some aspects, selecting the set of selected antigens comprises selecting antigens that have an increased likelihood of being capable of being presented to naïve T cells by professional antigen presenting cells (APCs) relative to unselected antigens based on the presentation model, optionally wherein the APC is a dendritic cell (DC). In some aspects, selecting the set of selected antigens comprises selecting antigens that have a decreased likelihood of being subject to inhibition via central or peripheral tolerance relative to unselected antigens based on the presentation model. In some aspects, selecting the set of selected antigens comprises selecting antigens that have a decreased likelihood of being capable of inducing an autoimmune response to normal tissue in the subject relative to unselected antigens based on the presentation model. In some aspects, exome or transcriptome SARS-CoV-2 nucleotide sequencing data is obtained by performing sequencing on a SARS-CoV-2 virus or SARS-CoV-2 infected tissue or cell. In some aspects, the sequencing is next generation sequencing (NGS) or any massively parallel sequencing approach.

In some aspects, the antigen cassette comprises junctional epitope sequences formed by adjacent sequences in the antigen cassette. In some aspects, at least one or each junctional epitope sequence has an affinity of greater than 500 nM for MHC. In some aspects, each junctional epitope sequence is non-self.

In some aspects, each of the MHC class I and/or MHC class II epitopes is predicted or validated to be capable of presentation by at least one HLA allele present in at least 5% of a population. In some aspects, each of the MHC class I and/or MHC class II epitopes is predicted or validated to be capable of presentation by at least one HLA allele, wherein each antigen/HLA pair has an antigen/HLA prevalence of at least 0.01% in a population. In some aspects, each of the MHC class I and/or MHC class II epitopes is predicted or validated to be capable of presentation by at least one HLA allele, wherein each antigen/HLA pair has an antigen/HLA prevalence of at least 0.1% in a population.

In some aspects, the antigen cassette does not encode a non-therapeutic MHC class I or class II epitope nucleic acid sequence comprising a translated, wild-type nucleic acid sequence, wherein the non-therapeutic epitope is predicted to be displayed on an MHC allele of the subject. In some aspects, the non-therapeutic predicted MHC class I or class II epitope sequence is a junctional epitope sequence formed by adjacent sequences in the antigen cassette.

In some aspects, the prediction is based on presentation likelihoods generated by inputting sequences of the non-therapeutic epitopes into a presentation model. In some aspects, an order of the at least one SARS-CoV-2 derived nucleic acid sequences in the antigen cassette is determined by a series of steps comprising: (a) generating a set of candidate antigen cassette sequences corresponding to different orders of the at least one SARS-CoV-2 derived nucleic acid sequences; (b) determining, for each candidate antigen cassette sequence, a presentation score based on presentation of non-therapeutic epitopes in the candidate antigen cassette sequence; and (c) selecting a candidate cassette sequence associated with a presentation score below a predetermined threshold as the antigen cassette sequence for an antigen vaccine.

Also provided for herein is a pharmaceutical composition any of the compositions provided herein and a pharmaceutically acceptable carrier. In some aspects, the composition further comprises an adjuvant. In some aspects, the composition further comprises an immune modulator. In some aspects, the immune modulator is an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof.

Also provided herein is an isolated nucleotide sequence or set of isolated nucleotide sequences comprising the antigen cassette of any of the compositions described herein and one or more elements obtained from the sequence of SEQ ID NO:3 or SEQ ID NO:5, optionally wherein the one or more elements are selected from the group consisting of the sequences necessary for nonstructural protein-mediated amplification, the 26S promoter nucleotide sequence, the poly(A) sequence, and the nsP1-4 genes of the sequence set forth in SEQ ID NO:3 or SEQ ID NO:5, and optionally wherein the nucleotide sequence is cDNA. In some aspects, the sequence or set of isolated nucleotide sequences comprises the antigen cassette of any of the above composition claims inserted at position 7544 of the sequence set forth in SEQ ID NO:6 or SEQ ID NO:7. In some aspects, the isolated sequence further comprises: a T7 or SP6 RNA polymerase promoter nucleotide sequence 5′ of the one or more elements obtained from the sequence of SEQ ID NO:3 or SEQ ID NO:5; and optionally, one or more restriction sites 3′ of the poly(A) sequence. In some aspects, the antigen cassette of any of the compositions provided herein is inserted at position 7563 of SEQ ID NO:8 or SEQ ID NO:9.

Also provided herein is an isolated nucleotide sequence or set of isolated nucleotide sequences comprising the antigen cassette of any of the compositions provided herein and one or more elements obtained from the sequence of SEQ ID NO:1 or SEQ ID NO:75, optionally wherein the one or more elements are selected from the group consisting of the chimpanzee adenovirus inverted terminal repeat (ITR), E1A, E1B, E2A, E2B, E3, E4, L1, L2, L3, L4, and L5 genes of the sequence set forth in SEQ ID NO:1, and optionally wherein the nucleotide sequence is cDNA. In some aspects, the sequence or set of isolated nucleotide sequences comprises the antigen cassette of any of the compositions provided herein inserted within the E1 deletion of the sequence set forth in SEQ ID NO:75. In some aspects, the isolated sequence further comprises: a T7 or SP6 RNA polymerase promoter nucleotide sequence 5′ of the one or more elements obtained from the sequence of SEQ ID NO:1 or SEQ ID NO:75; and optionally, one or more restriction sites 3′ of the poly(A) sequence.

Also provided herein is a vector or set of vectors comprising any of the isolated nucleotide sequences or set of isolated nucleotide sequences provided herein.

Also provided herein is an isolated cell comprising any of the isolated nucleotide sequences or set of isolated nucleotide sequences provided herein, optionally wherein the cell is a BHK-21, CHO, HEK293 or variants thereof, 911, HeLa, A549, LP-293, PER.C6, or AE1-2a cell.

Also provided herein is a kit comprising any of the compositions provided herein and instructions for use.

Also provided herein is a method for treating a SARS-CoV-2 infection or preventing a SARS-CoV-2 infection in a subject, the method comprising administering to the subject any of the compositions or pharmaceutical compositions provided herein. In some aspects, the SARS-CoV-2 derived nucleic acid sequence encodes at least one immunogenic polypeptide corresponding to a polypeptide encoded by a SARS-CoV-2 subtype the subject is infected with or at risk for infection by.

In some aspects, any of the methods described herein comprises a homologous prime/boost strategy. In some aspects, any of the methods described herein comprises a heterologous prime/boost strategy. In some aspects, the heterologous prime/boost strategy comprises an identical antigen cassette encoded by different vaccine platforms. In some aspects, the heterologous prime/boost strategy comprises different antigen cassettes encoded by the same vaccine platform. In some aspects, the heterologous prime/boost strategy comprises different antigen cassettes encoded by different vaccine platforms. In some aspects, the different antigen cassettes comprise a Spike-encoding cassette and a separate T cell epitope encoding cassette. In some aspects, the different antigen cassettes comprise cassettes encoding distinct epitopes and/or antigens derived from different isolates of SARS-CoV-2.

Also provided herein is a method for inducing an immune response in a subject, the method comprising administering to the subject any of the compositions or pharmaceutical compositions provided herein. In some aspects, the subject expresses at least one HLA allele predicted or known to present a MHC class I or MHC class II epitope encoded by the at least one SARS-CoV-2 derived nucleic acid sequence. In some aspects, the subject expresses at least one HLA allele predicted or known to present a MHC class I epitope encoded by the at least one SARS-CoV-2 derived nucleic acid sequence, and wherein the MHC class I epitope comprises at least one MHC class I epitope comprising a polypeptide sequence as set forth in Table A. In some aspects, the subject express at least one HLA allele predicted or known to present a MHC class II epitope encoded by the at least one SARS-CoV-2 derived nucleic acid sequence, and wherein the MHC class II epitope comprises at least one MHC class II epitope comprising a polypeptide sequence as set forth in Table B. In some aspects, the composition is administered intramuscularly (IM), intradermally (ID), subcutaneously (SC), or intravenously (IV). In some aspects, the composition is administered intramuscularly

In some aspects, the method further comprises administration of one or more immune modulators, optionally wherein the immune modulator is administered before, concurrently with, or after administration of the composition or pharmaceutical composition. In some aspects, the one or more immune modulators are selected from the group consisting of: an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof. In some aspects, the immune modulator is administered intravenously (IV), intramuscularly (IM), intradermally (ID), or subcutaneously (SC). In some aspects, the subcutaneous administration is near the site of the composition or pharmaceutical composition administration or in close proximity to one or more vector or composition draining lymph nodes.

In some aspects, the method further comprises administering to the subject a second vaccine composition. In some aspects, the second vaccine composition is administered prior to the administration of the first composition or pharmaceutical composition. In some aspects, the second vaccine composition is administered subsequent to the administration of any of the compositions or pharmaceutical compositions provided herein. In some aspects, the second vaccine composition is the same as the first composition or pharmaceutical composition administered. In some aspects, the second vaccine composition is different from the first composition or pharmaceutical composition administered. In some aspects, the second vaccine composition comprises a chimpanzee adenovirus vector encoding at least one SARS-CoV-2 derived nucleic acid sequence. In some aspects, the at least one SARS-CoV-2 derived nucleic acid sequence encoded by the chimpanzee adenovirus vector is the same as the at least one SARS-CoV-2 derived nucleic acid sequence of any of the compositions provided herein.

Also provided herein is a method of manufacturing the one or more vectors of any of the above composition claims, the method comprising: (a) obtaining a linearized DNA sequence comprising the backbone and the antigen cassette; (b) in vitro transcribing the linearized DNA sequence by addition of the linearized DNA sequence to an in vitro transcription reaction containing all the necessary components to transcribe the linearized DNA sequence into RNA, optionally further comprising in vitro addition of the m7g cap to the resulting RNA; and (c) isolating the one or more vectors from the in vitro transcription reaction. In some aspects, the linearized DNA sequence is generated by linearizing a DNA plasmid sequence or by amplification using PCR. In some aspects, the DNA plasmid sequence is generated using one of bacterial recombination or full genome DNA synthesis or full genome DNA synthesis with amplification of synthesized DNA in bacterial cells. In some aspects, isolating the one or more vectors from the in vitro transcription reaction involves one or more of phenol chloroform extraction, silica column based purification, or similar RNA purification methods.

Also provided herein is a method of manufacturing the composition of any of the above composition claims for delivery of the antigen expression system, the method comprising: (a) providing components for the nanoparticulate delivery vehicle; (b) providing the antigen expression system; and (c) providing conditions sufficient for the nanoparticulate delivery vehicle and the antigen expression system to produce the composition for delivery of the antigen expression system. In some aspects, the conditions are provided by microfluidic mixing.

Also provided herein is a method of manufacturing an adenovirus vector disclosed herein, the method comprising: obtaining a plasmid sequence comprising the at least one promoter sequence and the antigen cassette; transfecting the plasmid sequence into one or more host cells; and isolating the adenovirus vector from the one or more host cells.

In some aspects, isolating comprises: lysing the host cell to obtain a cell lysate comprising the adenovirus vector; and purifying the adenovirus vector from the cell lysate.

In some aspects, the plasmid sequence is generated using one of bacterial recombination or full genome DNA synthesis or full genome DNA synthesis with amplification of synthesized DNA in bacterial cells. In some aspects, the one or more host cells are at least one of CHO, HEK293 or variants thereof, 911, HeLa, A549, LP-293, PER.C6, and AE1-2a cells. In some aspects, purifying the adenovirus vector from the cell lysate involves one or more of chromatographic separation, centrifugation, virus precipitation, and filtration.

In some aspects, any of the above compositions further comprise a nanoparticulate delivery vehicle. The nanoparticulate delivery vehicle, in some aspects, may be a lipid nanoparticle (LNP). In some aspects, the LNP comprises ionizable amino lipids. In some aspects, the ionizable amino lipids comprise MC3-like (dilinoleylmethyl-4-dimethylaminobutyrate) molecules. In some aspects, the nanoparticulate delivery vehicle encapsulates the antigen expression system.

In some aspects, any of the above compositions further comprise a plurality of LNPs, wherein the LNPs comprise: the antigen expression system; a cationic lipid; a non-cationic lipid; and a conjugated lipid that inhibits aggregation of the LNPs, wherein at least about 95% of the LNPs in the plurality of LNPs either: have a non-lamellar morphology; or are electron-dense.

In some aspects, the non-cationic lipid is a mixture of (1) a phospholipid and (2) cholesterol or a cholesterol derivative.

In some aspects, the conjugated lipid that inhibits aggregation of the LNPs is a polyethyleneglycol (PEG)-lipid conjugate. In some aspects, the PEG-lipid conjugate is selected from the group consisting of: a PEG-diacylglycerol (PEG-DAG) conjugate, a PEG dialkyloxypropyl (PEG-DAA) conjugate, a PEG-phospholipid conjugate, a PEG-ceramide (PEG-Cer) conjugate, and a mixture thereof. In some aspects the PEG-DAA conjugate is a member selected from the group consisting of: a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, a PEG-distearyloxypropyl (C₁₈) conjugate, and a mixture thereof.

In some aspects, the antigen expression system is fully encapsulated in the LNPs.

In some aspects, the non-lamellar morphology of the LNPs comprises an inverse hexagonal (H_{II}) or cubic phase structure.

In some aspects, the cationic lipid comprises from about 10 mol % to about 50 mol % of the total lipid present in the LNPs. In some aspects, the cationic lipid comprises from about 20 mol % to about 50 mol % of the total lipid present in the LNPs. In some aspects, the cationic lipid comprises from about 20 mol % to about 40 mol % of the total lipid present in the LNPs.

In some aspects, the non-cationic lipid comprises from about 10 mol % to about 60 mol % of the total lipid present in the LNPs. In some aspects, the non-cationic lipid comprises from about 20 mol % to about 55 mol % of the total lipid present in the LNPs. In some aspects, the non-cationic lipid comprises from about 25 mol % to about 50 mol % of the total lipid present in the LNPs.

In some aspects, the conjugated lipid comprises from about 0.5 mol % to about 20 mol % of the total lipid present in the LNPs. In some aspects, the conjugated lipid comprises from about 2 mol % to about 20 mol % of the total lipid present in the LNPs. In some aspects, the conjugated lipid comprises from about 1.5 mol % to about 18 mol % of the total lipid present in the LNPs.

In some aspects, greater than 95% of the LNPs have a non-lamellar morphology. In some aspects, greater than 95% of the LNPs are electron dense.

In some aspects, any of the above compositions further comprise a plurality of LNPs, wherein the LNPs comprise: a cationic lipid comprising from 50 mol % to 65 mol % of the total lipid present in the LNPs; a conjugated lipid that inhibits aggregation of LNPs comprising from 0.5 mol % to 2 mol % of the total lipid present in the LNPs; and a non-cationic lipid comprising either: a mixture of a phospholipid and cholesterol or a derivative thereof, wherein the phospholipid comprises from 4 mol % to 10 mol % of the total lipid present in the LNPs and the cholesterol or derivative thereof comprises from 30 mol % to 40 mol % of the total lipid present in the LNPs; a mixture of a phospholipid and cholesterol or a derivative thereof, wherein the phospholipid comprises from 3 mol % to 15 mol % of the total lipid present in the LNPs and the cholesterol or derivative thereof comprises from 30 mol % to 40 mol % of the total lipid present in the LNPs; or up to 49.5 mol % of the total lipid present in the LNPs and comprising a mixture of a phospholipid and cholesterol or a derivative thereof, wherein the cholesterol or derivative thereof comprises from 30 mol % to 40 mol % of the total lipid present in the LNPs.

In some aspects, any of the above compositions further comprise a plurality of LNPs, wherein the LNPs comprise: a cationic lipid comprising from 50 mol % to 85 mol % of the total lipid present in the LNPs; a conjugated lipid that inhibits aggregation of LNPs comprising from 0.5 mol % to 2 mol % of the total lipid present in the LNPs; and a non-cationic lipid comprising from 13 mol % to 49.5 mol % of the total lipid present in the LNPs.

In some aspects, the phospholipid comprises dipalmitoylphosphatidylcholine (DPPC), distearoylphosphatidylcholine (DSPC), or a mixture thereof.

In some aspects, the conjugated lipid comprises a polyethyleneglycol (PEG)-lipid conjugate. In some aspects, the PEG-lipid conjugate comprises a PEG-diacylglycerol (PEG-DAG) conjugate, a PEG-dialkyloxypropyl (PEG-DAA) conjugate, or a mixture thereof. In some aspects, the PEG-DAA conjugate comprises a PEG-dimyristyloxypropyl (PEG-DMA) conjugate, a PEG-distearyloxypropyl (PEG-DSA) conjugate, or a mixture thereof. In some aspects, the PEG portion of the conjugate has an average molecular weight of about 2,000 daltons.

In some aspects, the conjugated lipid comprises from 1 mol % to 2 mol % of the total lipid present in the LNPs.

In some aspects, the LNP comprises a compound having a structure of:

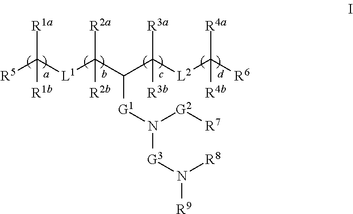

or a pharmaceutically acceptable salt, tautomer, prodrug or stereoisomer thereof, wherein: L¹ and L² are each independently —O(C═O)—, —(C═O)O—, —C(═O)—, —O—, —S(O)ₓ—, —S—S—, —C(═O)S—, —SC(═O)—, —R^{a}C(═O)—, —C(═O) R^{a}—, —R^{a}C(═O) R^{a}—, —OC(═O) R^{a}—, —R^{a}C(═O)O— or a direct bond; G¹ is Cᵢ-C₂ alkylene, —(C═O)—, —O(C═O)—, —SC(═O)—, —R^{a}C(═O)— or a direct bond: —C(═O)—, —(C═O)O—, —C(═O)S—, —C(═O) R^{a}— or a direct bond; G is Ci-C₆ alkylene; R^{a} is H or C1-C12 alkyl; R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond; R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond; R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond; R^{4a} and R^{4b} are, at each occurrence, independently either: (a) H or C1-C12 alkyl; or (b) R^{4a} is H or C1-C12 alkyl, and R^{4b} together with the carbon atom to which it is bound is taken together with an adjacent R^{4b} and the carbon atom to which it is bound to form a carbon-carbon double bond; R⁵ and R⁶ are each independently H or methyl; R⁷ is C4-C20 alkyl; R⁸ and R⁹ are each independently C1-C12 alkyl; or R⁸ and R⁹, together with the nitrogen atom to which they are attached, form a 5, 6 or 7-membered heterocyclic ring; a, b, c and d are each independently an integer from 1 to 24; and x is 0, 1 or 2.

In some aspects, the LNP comprises a compound having a structure of Formula II:

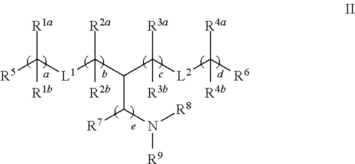

or a pharmaceutically acceptable salt, tautomer, prodrug or stereoisomer thereof, wherein: L¹ and L² are each independently —O(C═O)—, —(C═O)O— or a carbon-carbon double bond; R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond; R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond; R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond; R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond; R⁵ and R⁶ are each independently methyl or cycloalkyl; R⁷ is, at each occurrence, independently H or C₁-C₁₂ alkyl; R⁸ and R⁹ are each independently unsubstituted C1-C12 alkyl; or R⁸ and R⁹, together with the nitrogen atom to which they are attached, form a 5, 6 or 7-membered heterocyclic ring comprising one nitrogen atom; a and d are each independently an integer from 0 to 24; b and c are each independently an integer from 1 to 24; and e is 1 or 2, provided that: at least one of R^{1a}, R^{2a}, R^{3a} or R^{4a} is C1-C12 alkyl, or at least one of L¹ or L² is —O(C═O)— or —(C═O)O—; and R^{1a} and R^{1b} are not isopropyl when a is 6 or n-butyl when a is 8.

In some aspects, any of the above compositions further comprise one or more excipients comprising a neutral lipid, a steroid, and a polymer conjugated lipid. In some aspects, the neutral lipid comprises at least one of 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), and 1,2-Dioleoyl-sn-glycero-3-phosphoethanolamine (DOPE). In some aspects, the neutral lipid is DSPC.

In some aspects, the molar ratio of the compound to the neutral lipid ranges from about 2:1 to about 8:1.

In some aspects, the steroid is cholesterol. In some aspects, the molar ratio of the compound to cholesterol ranges from about 2:1 to 1:1.

In some aspects, the polymer conjugated lipid is a pegylated lipid. In some aspects, the molar ratio of the compound to the pegylated lipid ranges from about 100:1 to about 25:1. In some aspects, the pegylated lipid is PEG-DAG, a PEG polyethylene (PEG-PE), a PEG-succinoyl-diacylglycerol (PEG-S-DAG), PEG-cer or a PEG dialkyoxypropylcarbamate. In some aspects, the pegylated lipid has the following structure III:

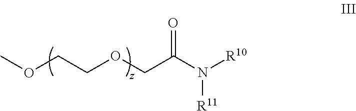

or a pharmaceutically acceptable salt, tautomer or stereoisomer thereof, wherein: R¹⁰ and R¹¹” are each independently a straight or branched, saturated or unsaturated alkyl chain containing from 10 to 30 carbon atoms, wherein the alkyl chain is optionally interrupted by one or more ester bonds; and z has a mean value ranging from 30 to 60. In some aspects, R¹⁰ and R¹¹ are each independently straight, saturated alkyl chains having 12 to 16 carbon atoms. In some aspects, the average z is about 45. start here

In some aspects, the LNP self-assembles into non-bilayer structures when mixed with polyanionic nucleic acid. In some aspects, the non-bilayer structures have a diameter between 60 nm and 120 nm. In some aspects, the non-bilayer structures have a diameter of about 70 nm, about 80 nm, about 90 nm, or about 100 nm. In some aspects, wherein the nanoparticulate delivery vehicle has a diameter of about 100 nm.

Also provided for herein is a vector or set of vectors comprising any of the nucleotide sequence described herein. Also disclosed herein is a vector comprising an isolated nucleotide sequence disclosed herein.

Also provided for herein is an isolated cell comprising any of the nucleotide sequences or set of isolated nucleotide sequences described herein, optionally wherein the cell is a BHK-21, CHO, HEK293 or variants thereof, 911, HeLa, A549, LP-293, PER.C6, or AE1-2a cell.

Also provided for herein is a kit comprising any of the compositions described herein and instructions for use. Also disclosed herein is a kit comprising a vector or a composition disclosed herein and instructions for use.

Also provided for herein is a method for treating a subject suffering from Covid-19, the method comprising administering to the subject any of the compositions or any of the pharmaceutical compositions described herein.

Also provided for herein is a method for treating a subject infected with or at risk for infection by SARS-CoV-2, the method comprising administering to the subject any of the compositions or any of the pharmaceutical compositions described herein.

Also provided for herein is a method for stimulating an immune response in a subject, the method comprising administering to the subject any of the compositions or any of the pharmaceutical compositions described herein.

Also disclosed herein is a method for treating a subject, the method comprising administering to the subject a vector disclosed herein or a pharmaceutical composition disclosed herein.

Also disclosed herein is a method of manufacturing the one or more vectors of any of the above compositions.

Also disclosed herein is a method of manufacturing any of the compositions disclosed herein.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 presents a schematic of the SARS-CoV-2 genome structure depicting the at least 14 open reading frames (ORF) identified in. Figure adapted from Zhou et al. (2020) [A pneumonia outbreak associated with a new coronavirus of probable bat origin. Nature, 579 (January)].

FIG. 2 depicts the 16 cleavage products of the replicase ORFlab and related information. Figure adapted from Wu et al. (2020). [A new coronavirus associated with human respiratory disease in China. Nature, 579 (January)].

FIG. 3 depicts the general vaccination approach of producing a balanced immune response inducing both neutralizing antibodies (from B cells) as well as effector and memory CD8+ T cell responses for maximum efficacy. SARS-CoV-2 genome structure adapted from Zhou et al. (2020) [A pneumonia outbreak associated with a new coronavirus of probable bat origin. Nature, 579 (January)].

FIG. 4 demonstrates the known prevalence of the wildtype and D614G variant SARS-Cov-2 Spike protein over time across various geographic locations.

FIG. 5 demonstrates coverage of cassettes encoding only Spike or encoding Spike and the additional predicted concatenated T cell epitopes over the four populations shown. The first column demonstrates the number of SARS-CoV-2 epitopes predicted to be presented and the second column demonstrates the expected number of presented epitopes, based on a 0.2 PPV. Each row shows the protection coverage of each population if a certain number of epitopes is used.

FIG. 6A illustrates the number of predicted epitopes presented by each MHC class II allele separately for the Spike protein or the additional predicted concatenated T cell epitopes.

FIG. 6B illustrates the number of the number of SARS-CoV-2 epitopes predicted to be presented over the four populations shown from cassettes encoding only Spike (top panel) or encoding Spike and the additional predicted concatenated T cell epitopes (bottom panel).

FIG. 7A presents the number of training samples containing Class I alleles (with at least 10 samples).

FIG. 7B presents a histogram depicting the number of training samples per Class I allele versus the number of alleles.

FIG. 8A shows a Western blot using an anti-Spike S2 antibody for Spike expression in vectors encoding various Spike variations.

FIG. 8B shows a Western blot using an anti-Spike S1 antibody for Spike expression in vectors encoding various Spike variations.

FIG. 8C shows a Western blot using an anti-Spike S1 antibody for Spike expression in vectors encoding full-length Spike, Spike S1 alone, or Spike S2 alone.

FIG. 8D shows a Western blot using an anti-Spike S2 antibody for Spike expression in vectors encoding full-length Spike, Spike S1 alone, or Spike S2 alone.

FIG. 9 shows a Western blot using an anti-Spike S2 antibody for Spike expression in vectors encoding various sequence-optimized Spike variations.

FIG. 10A depicts a schematic of PCR-based assay to assess RNA splicing of SARS-CoV-2 transcripts.

FIG. 10B shows PCR amplicons for encoded Spike proteins. Left panel depicts amplicons from cDNA templates from infected 293 cells (“ChAd-Spike (IDT) cDNA”) or from the plasmid encoding the SARS-CoV-2 Spike cassette (“Spike Plasmid”). Right panel depicts amplicons from the cDNA of 293 cells infected with a vector encoding Spike S1 alone (“SpikeS1”) or full-length Spike (“Spike”).

FIG. 11 shows PCR amplicons for encoded Spike proteins from the cDNA of 293 cells infected with vector encoding various Spike variations.

FIG. 12 presents estimated coverages for the percentage of the indicated ancestry populations having at least one HLA estimated to receive at least one immunogenic epitope encoded by TCE5, where receipt of the immunogenic peptide presentation is considered to occur when an individual's HLA is either (1) known to present an encoded epitope (“validated epitope”), or (2) predicted to present at least 4 (Col. 1), 5 (Col. 2), 6 (Col. 3), or 7 (Col. 4) encoded epitopes (“predicted epitope”; EDGE score >0.01). FA=African American, API=Asian or Pacific Islander, EUR=European, HIS=Hispanic

FIG. 13A presents T cell responses (left panel), Spike-specific IgG antibodies (middle panel) and neutralizing antibodies (right panel) following administration of ChAdV-platforms with Spike-encoding cassettes featuring different sequence optimizations “IDTSpike_{g}” (shown as “Spike V1” or “v1”) or “CTSpike_{g}” (shown as “Spike V2” or “v2”). Balb/c mice immunized with 1×10¹¹ VP ChAdV-based vaccine platform.

FIG. 13B presents T cell responses (left panel), Spike-specific IgG antibodies (middle panel) and neutralizing antibodies (right panel) following administration of SAM-platforms with Spike-encoding cassettes featuring different sequence optimizations “IDTSpike_{g}” (shown as “Spike V1” or “vi”) or “CTSpike_{g}” (shown as “Spike V2” or “v2”). Balb/c mice immunized with 10 μg SAM-based vaccine platform.

FIG. 14 presents Spike-specific IgG antibody production following administration of either ChAdV-platform (left panel) or SAM-platform (right panel) with unmodified or modified (“CTSpikeF2P_{g}” shown as “SpikeF2P”) Spike-encoding cassettes (all vectors utilize Spike sequence v2). Balb/c mice immunized with 1×10¹¹ VP ChAdV-based vaccine platform or 10 μg SAM-based vaccine platform, as indicated.

FIG. 15A presents T cell responses to Spike (left panel) and T cell responses to the encoded T cell epitopes (right panel) following administration of ChAdV-platforms with a modified Spike-encoding only cassette (“CTSpikeF2P_{g}” shown as “Spike”) and modified Spike together with additional non-Spike T cell epitopes encoded TCE5 (shown as “Spike TCE”). Balb/c mice immunized with 1×10¹¹ VP ChAdV-based vaccine platform. Shown is IFNγ ELISpot, 2 weeks post immunization. T cell response to overlapping peptide pools spanning either Spike, Nucleocapsid, or Orf3a.

FIG. 15B presents T cell responses to Spike (left panel) and T cell responses to the encoded T cell epitopes (right panel) following administration of SAM-platforms with a modified Spike-encoding only cassette (“CTSpikeF2P_{g}” shown as “Spike”) and modified Spike together with additional non-Spike T cell epitopes encoded TCE5 (shown as “TCE Spike”). Balb/c mice immunized with 10 μg SAM-based vaccine platform. Shown is IFNγ ELISpot, 2 weeks post immunization. T cell response to overlapping peptide pools spanning either Spike, Nucleocapsid, or Orf3a.

FIG. 16A presents T cell responses to Spike (top panel; IFNg ELISpot. Sum of response to 8 overlapping peptide pools spanning Spike antigen), T cell responses to the encoded cell epitopes (middle panel; IFNg ELISpot. Sum of response to 3 overlapping peptide pools spanning NCap, Membrane, and Orf3a), and Spike-specific IgG antibodies (bottom panel; S1 IgG binding measured by MSD ELISA. Interpolated endpoint titer. Geomean, geometric SD) following immunization with SAM constructs including “IDTSpike_{g}” alone (left columns), IDTSpike_{g} expressed from a first subgenomic promoter followed by TCE5 expressed from a second subgenomic promoter (middle columns), or TCE5 expressed from a first subgenomic promoter followed by IDTSpike_{g} expressed from a second subgenomic promoter (right columns). For T cell responses, Balb/c mice were immunized with 10 ug of each vaccine, n=6/group. Splenocyte isolation at 2-weeks post immunization. For IgG response, Balb/c mice immunized with 10 ug of each vaccine, n=4/group. Serum collected and analyzed at 4-weeks post immunization.

FIG. 16B presents T cell responses to Spike (top panel; IFNg ELISpot. Sum of response to 8 overlapping peptide pools spanning Spike antigen), T cell responses to the encoded T cell epitopes (middle panel; IFNg ELISpot. Sum of response to 3 overlapping peptide pools spanning NCap, Membrane, and Orf3a), and Spike-specific IgG antibodies (bottom panel; S1 IgG binding measured by MSD ELISA. Interpolated endpoint titer. Geomean, geometric SD) following immunization with SAM constructs including “IDTSpike_{g}” alone (first column), IDTSpike_{g} expressed from a first subgenomic promoter followed by TCE6 or TCE7 expressed from a second subgenomic promoter (columns 2 and 4, respectively), or TCE6 or TCE7 expressed from a first subgenomic promoter followed by IDTSpike_{g} expressed from a second subgenomic promoter (columns 3 and 5, respectively). For T cell responses, Balb/c mice were immunized with 10 ug of each vaccine, n=6/group. Splenocyte isolation at 2-weeks post immunization. For IgG response, Balb/c mice immunized with 10 ug of each vaccine, n=4/group. Serum collected and analyzed at 4-weeks post immunization.

FIG. 16C presents T cell responses to Spike (top panel; IFNg ELISpot. Sum of response to 2 overlapping peptide pools spanning Spike antigen), T cell responses to the encoded T cell epitopes (middle panel; IFNg ELISpot. Sum of response to 2 overlapping peptide pools spanning NCap and Orf3a), and Spike-specific IgG antibodies (bottom panel; S1 IgG binding measured by MSD ELISA. Interpolated endpoint titer. Geomean, geometric SD) following immunization with SAM constructs including “CTSpike_{g}” alone (first column), CTSpike_{g} expressed from a first subgenomic promoter followed by TCE5 or TCE8 expressed from a second subgenomic promoter (columns 2 and 4, respectively), or TCE5 or TCE8 expressed from a first subgenomic promoter followed by CTSpike_{g} expressed from a second subgenomic promoter (columns 3 and 5, respectively). For T cell responses, Balb/c mice were immunized with 10 ug of each vaccine, n=6/group. Splenocyte isolation at 2-weeks post immunization. For IgG response, Balb/c mice immunized with 10 ug of each vaccine, n=4/group. Serum collected and analyzed at 4-weeks post immunization.

FIG. 17A presents a map of sequences included in TCE10 for Nucleocapsid, including frames with flanking sequences, validated epitopes, predicted epitopes, mutations, and overlap between frames and mutations.

FIG. 17B presents a map of sequences included in TCE10 for ORF3a, including frames with flanking sequences, validated epitopes, predicted epitopes, mutations, and overlap between frames and mutations.

FIG. 17C presents a map of sequences included in TCE10 for nsp3, including frames with flanking sequences, validated epitopes, predicted epitopes, mutations, and overlap between frames and mutations.

FIG. 17D presents a map of sequences included in TCE10 for Membrane, including frames with flanking sequences, validated epitopes, predicted epitopes, mutations, and overlap between frames and mutations.

FIG. 17E presents a map of sequences included in TCE10 for nsp4, including frames with flanking sequences, validated epitopes, predicted epitopes, mutations, and overlap between frames and mutations.

FIG. 17F presents a map of sequences included in TCE10 for nsp12, including frames with flanking sequences, validated epitopes, predicted epitopes, mutations, and overlap between frames and mutations.

FIG. 18A presents a map of sequences included in TCE9 for nsp12, including frames with flanking sequences, validated epitopes, predicted epitopes, mutations, and overlap between frames and mutations.

FIG. 18B presents a map of sequences included in TCE9 for nsp4, including frames with flanking sequences, validated epitopes, predicted epitopes, mutations, and overlap between frames and mutations.

FIG. 18C presents a map of sequences included in TCE9 for Membrane, including frames with flanking sequences, validated epitopes, predicted epitopes, mutations, and overlap between frames and mutations.

FIG. 18D presents a map of sequences included in TCE9 for nsp3, including frames with flanking sequences, validated epitopes, predicted epitopes, mutations, and overlap between frames and mutations.

FIG. 18E presents a map of sequences included in TCE9 for ORF3a, including frames with flanking sequences, validated epitopes, predicted epitopes, mutations, and overlap between frames and mutations.

FIG. 18F presents a map of sequences included in TCE9 for Nucleocapsid, including frames with flanking sequences, validated epitopes, predicted epitopes, mutations, and overlap between frames and mutations.

FIG. 18G presents a map of sequences included in TCE9 for nsp6, including frames with flanking sequences, validated epitopes, predicted epitopes, mutations, and overlap between frames and mutations.

FIG. 19A presents a map of sequences included in TCE11 for nsp12, including frames with flanking sequences, validated epitopes, predicted epitopes, mutations, and overlap between frames and mutations.

FIG. 19B presents a map of sequences included in TCE11 for Membrane, including frames with flanking sequences, validated epitopes, predicted epitopes, mutations, and overlap between frames and mutations.

FIG. 19C presents a map of sequences included in TCE11 for nsp4, including frames with flanking sequences, validated epitopes, predicted epitopes, mutations, and overlap between frames and mutations.

FIG. 19D presents a map of sequences included in TCE11 for nsp3, including frames with flanking sequences, validated epitopes, predicted epitopes, mutations, and overlap between frames and mutations.

FIG. 20 presents the percentages of shared candidate 9-mer epitope distribution between SARS-CoV-2 and SARS-CoV (left panel) and between SARS-CoV-2 and MERS (right panel).

FIG. 21 illustrates homologous and heterologous prime/boost regimens in Indian rhesus macaques assessing ChAdV and SAM vaccine platforms encoding different isolates of the SARS-CoV-2 Spike protein.

FIG. 22A presents T cell responses across multiple Spike T cell epitope pools (top panel; Mean+−SE for each pool), T cell responses for individual NHPs directed to a single large Spike T cell epitope pool over time (middle panel), and Spike-specific IgG antibody titers over time (bottom panel) for Group 1. n=5 NHPs

FIG. 22B presents T cell responses across multiple Spike T cell epitope pools (top panel; Mean+−SE for each pool), T cell responses for individual NHPs directed to a single large Spike T cell epitope pool over time (middle panel), and Spike-specific IgG antibody titers over time (bottom panel) for Group 2. n=5 NHPs

FIG. 22C presents T cell responses across multiple Spike T cell epitope pools (top panel; Mean+−SE for each pool), T cell responses for individual NHPs directed to a single large Spike T cell epitope pool over time (middle panel), and Spike-specific IgG antibody titers over time (bottom panel) for Group 5. n=5 NHPs

FIG. 22D presents T cell responses across multiple Spike T cell epitope pools (top panel; Mean+−SE for each pool), T cell responses for individual NHPs directed to a single large Spike T cell epitope pool over time (middle panel), and Spike-specific IgG antibody titers over time (bottom panel) for Group 6. n=5 NHPs

FIG. 23 presents summaries of T cell responses for individual NHPs directed to a single large Spike T cell epitope pool over time (top panel), T cell responses to the TCE5-encoded epitopes (middle panel), and Spike-specific IgG antibody titers over time (bottom panel) for Group 1. n=5 NHPs

FIG. 24 presents neutralizing antibody production to both the D614G pseudovirus (left panels) and B.1.351 pseudovirus (right panels) following Boost 1 (left columns) and Boost 2 (right columns) for each of the NHP Groups.

FIG. 25 presents neutralizing antibody production comparing the relative Nab titer levels against each of the pseudoviruses following Boost 1 (top panels) and following Boost 2 (bottom panels).

FIG. 26 shows a dosing regimen for Rhesus macaques immunized twice with SAM encoding SARS-CoV-2 Spike antigen at specified dose of either 30 μg or 300 μg. Shown are PBMCs assessed by ex-vivo IFNγ ELISpot following overnight stimulation with Spike-specific overlapping peptide pool (left panel) and neutralizing antibodies measured in serum at study week 8 by pseudovirus neutralization assay (right panel).

FIG. 27 presents impact of Omicron variant mutations on epitope coverage for TCE5, TCE9, and TCE11.

FIG. 28A shows a schematic of SARS-CoV-2 vaccine evaluation and challenge in rhesus macaques. Filled circles represent ChAd immunization (5×10¹¹ VP), squares represent SAM vaccination at varying doses, and open circle represents challenge with SARS-CoV-2 virus. N=5/group.

FIG. 28B shows spike S1 IgG endpoint titers, geometric mean (annotated) and geometric SD. LOD=50, samples below LOD set to ½ LOD.

FIG. 28C shows IFNγ ELISpot (SFU/10⁶ PBMCs) at specified timepoint post immunization, following overnight stimulation with overlapping peptide pool spanning spike antigen. Mean (annotated) +/−SEM.

FIG. 28D shows IL-4 vs IFNγ ELISpot of PBMCs assessed 1-week post boost immunization. Individual animal values (n=5/group). Background corrected values. Diagonal line represents unity line.

FIG. 28E shows pseudovirus neutralization titers (50% inhibition, NT50) assessed in sera at specified timepoint post immunization. LOD=50, samples <LOD set to ½ LOD. Geometric mean (annotated) and SD. HCS=human convalescent serum assessed by same assay. C═PBS control injected animals assessed 8 weeks post injection.

FIG. 28F shows live virus microneutralizing titer (50% inhibition, NT50) at specified timepoint post immunization and post SARS-CoV-2 challenge, geometric mean (annotated) and geometric SD. LOD=20, samples below LOD set to ½ LOD.

FIG. 29 shows a scatterplot comparing NT50 titers measured by pseudovirus neutralization assay (PNA) and live virus microneutralization assay (MNA) for all samples that were assessed by both assays (excluding baseline samples). Dashed line represents unity. LOD for MNA assay=20, LOD for PNA assay=50. Solid line represents non-linear regression analysis best fit.

FIG. 30 shows Serum IFNα2α concentration assessed by MSD in rhesus macaques (n=5/group) 8 hours post immunization with SAM-Spike(V2)-F2P at the specified dose. Median, IQR, range.

FIG. 31A shows viral replication in vaccinated NHP following SARS-CoV-2 challenge as assessed by subgenomic RNA levels determined by RT-qPCR at specified timepoint post SARSCoV-2 challenge for each animal in bronchial alveolar lavage. LOD=422, samples below LOD set to ½ LOD. Geometric mean and SD. Numbers above each bar are numbers of NHP with viral load levels >LOD.

FIG. 31B shows viral replication in vaccinated NHP following SARS-CoV-2 challenge as assessed by subgenomic RNA levels determined by RT-qPCR at specified timepoint post SARSCoV-2 challenge for each animal in oropharyngeal swab. LOD=422, samples below LOD set to ½ LOD. Geometric mean and SD. Numbers above each bar are numbers of NHP with viral load levels >LOD.

FIG. 31C shows viral replication in vaccinated NHP following SARS-CoV-2 challenge as assessed by subgenomic RNA levels determined by RT-qPCR at specified timepoint post SARSCoV-2 challenge for each animal in nasal swab. LOD=422, samples below LOD set to ½ LOD. Geometric mean and SD. Numbers above each bar are numbers of NHP with viral load levels >LOD.

FIG. 32 shows viral replication assessed in vaccinated NHP following SARS-CoV-2 challenge. Peak subgenomic RNA levels in bronchial alveolar lavage for each animal determined by RT-qPCR between day 3 and day 10 post SARS-CoV-2 challenge (left panel). Peak subgenomic RNA levels in nasal swab for each animal determined by RT-qPCR between day 2 and day 10 post SARS-CoV-2 challenge (right panel). LOD=422, samples below LOD set to ½ LOD. LLOQ=3,881. Bars represent median. Statistical analyses: Mann-Whitney test for each group compared to unvaccinated control group.

FIG. 33A shows total genomic RNA levels (N1) determined by RT-qPCR at specified timepoint post SARS-CoV-2 challenge for each animal in bronchial alveolar lavage. LOD=230, samples below LOD set to ½ LOD. Geometric mean and SD.

FIG. 33B shows total genomic RNA levels (N1) determined by RT-qPCR at specified timepoint post SARS-CoV-2 challenge for each animal in oropharyngeal swab. LOD=230, samples below LOD set to ½ LOD. Geometric mean and SD.

FIG. 33C shows total genomic RNA levels (N1) determined by RT-qPCR at specified timepoint post SARS-CoV-2 challenge for each animal in nasal swab. LOD=230, samples below LOD set to ½ LOD. Geometric mean and SD.

FIG. 34 shows a schematic of vaccine candidate GRT-R910 (SAM-SGP1-TCE5-SGP2-CTSpike_{G}F2P) and its antigenic coverage.

FIG. 35 shows a schematic of the cohorts for the clinical trial (GO-009) assessing vaccine candidate GRT-R910 (SAM-SGP1-TCE5-SGP2-CTSpike_{G}F2P).

FIG. 36 shows a summary of the participant demographics for the clinical trial (GO-009) assessing vaccine candidate GRT-R910 (SAM-SGP1-TCE5-SGP2-CTSpike_{G}F2P). Subjects enrolled in cohorts 1 and 2 must have had negative SARS-CoV-2 serology whereas subjects enrolled in cohorts 3 and 4 may have had positive SARS-CoV-2 serology provided that they did not have symptoms consistent with SARS-CoV-2 infection within 112 days prior to enrollment Subjects without positive N-serology had SARS-Cov-2 infection occurred within 6 months (subject reported in CRF), so the status captured as convalescent. Subject 0014 in Cohort 1 received a BNT vaccine on Feb. 13, 2022 after receiving R910 on Sep. 30, 2021. Subject 0024 in Cohort 2 received a BNT vaccine on Feb. 24, 2022 after receiving R910 on Nov. 18, 2021. ¹Status at baseline.

FIG. 37 shows safety and reactogenicity summaries for the clinical trial (GO-009) assessing vaccine candidate GRT-R910 (SAM-SGP1-TCE5-SGP2-CTSpike_{G}F2P) following dose 1 (top panel) and dose 2 (bottom panel) as a comparison between 10 μg and 30 μg doses.

FIG. 38 shows safety and reactogenicity summaries for the clinical trial (GO-009) assessing vaccine candidate GRT-R910 (SAM-SGP1-TCE5-SGP2-CTSpike_{G}F2P) broken down by cohort.

FIG. 39 shows nAb response against Wild Type, Beta, Delta, and Omicron variants of SARS-CoV-2 in healthy adults (≥60 years old) following a single 10 μg samRNA boost. *ID50=Median infective dose, **Geomean ID50 titer values notated—not studied head-to-head directly. Treatment day=day 1 GRTS samRNA boost dose was administered. Boxes and horizontal bars denote interquartile range (IQR) and median neutralization, respectively. Whisker endpoints are equal to the maximum and minimum values below or above the median +/−1.5×IQR.

FIG. 40 shows anti-Spike IgG response as assessed by ELISA against Wild Type, Beta, Delta, and Omicron variants of SARS-CoV-2 in healthy adults (≥60 years old) following a single 10 μg samRNA boost. *Geomean AU/ml indicated. Treatment day=day 1 GRTS samRNA boost dose was administered. Boxes and horizontal bars denote interquartile range (IQR) and median binding, respectively. Whisker endpoints are equal to the maximum and minimum values below or above the median +/−1.5×IQR.

FIG. 41 shows titer levels for Spike IgG (top panel) and nAb (bottom panel) following single doses of 10 μg or 30 μg samRNA.

FIG. 42 shows pre and post boost titer levels for Spike IgG (top panel) and nAb (bottom panel) following single doses of 10 μg or 30 μg samRNA. AU/mL=Arbitrary units per mL; ID50 titer=Median infective dose; Geometric mean of AU/mL or ID50 titer values notated. Treatment day=day 1 single dose (10 μg or 30 μg) of GRT-R910 samRNA was administered. Boxes and horizontal bars denote interquartile range (IQR) and median values, respectively. Whisker endpoints are equal to the maximum and minimum values. 17 shown, 10 subjects from cohort 1 (10 μg) and 7 subjects from cohort 2 (30 μg, denoted by black circles).

FIG. 43 shows neutralizing antibodies against multiple SARS-CoV-2 variants of interest over a time course of 6-months following a single boost of 10 μg or 30 μg in healthy adults (≥60 years old). 7 previously vaccinated subjects 60+ years of age receiving one 10 mcg or 30 mcg dose of samRNA post ChAdOx1 series. Subjects G09-101-0014 & G09-101-0024 were excluded at Day 180 due to positive COVID diagnosis (D108 & D110) and BNT162b2 vaccination (D137 & D135).

FIG. 44 shows Spike IgG (top panel) and neutralizing (bottom panel) antibodies against multiple SARS-CoV-2 variants of interest over a time course of 6-months following a single boost of 10 μg or 30 μg in healthy adults (≥60 years old). AU/ml=Arbitrary units per ml; ID:50 titer=Mean infective dose; Geometric mean of AU/ml or ID50 titer values notated. Treatment day=day 1 single dose (10 μg or 30 μg) of GRT-R910 samRNA was administered. Boxes and horizontal bars denote interquartile range (IQR) and median values, respectively. Whisker endpoints are equal to the maximum and minimum values. 7 subjects from cohorts 1 (10 μg) and 2 (30 μg, subjects denoted by black circles). Two subject timepoints were excluded at D180 due to positive COV/D19 diagnosis (D108 & D110) and BNT162b2 vaccination (D137 & 135) prior to D180.

FIG. 45 shows T cell responses after a single boost with the samRNA against Spike and non-Spike T cell epitope (TCE) regions. SFU=Spot forming units per 10⁶ cells; Treatment day=day 1 single dose (10 μg or 30 μg) of GRT-R910 samRNA was administered. Peak=maximum T cell response at either day RT-R910 dose. Boxes and horizontal bars denote interquartile range (IQR) and median values, respectively. Whisker endpoints are equal to the maximum and minimum values. 17 subjects day 8 or day 29 after shown, 10 subjects from cohort 1 (10 μg) and 7 subjects from cohort 2 (30 μg, denoted by black circles). Ex vivo=overnight ELISpot (Spike only). Responses to Nucleocapsid, Membrane and ORF3a are after in vitro expansion. Circles denote positive responses, triangles denote responses <LOD/<2×DMSO control.

FIG. 46 shows T cell responses against Spike (top panel) and TCE epitopes (bottom panel) over a time course. SFU=Spot forming units per 10⁶ cells; Treatment day=day 1 single dose (10 μg or 30 μg) of GRT-R910 samRNA was administered. Boxes and horizontal bars denote interquartile range (IQR) and median values, respectively. Whisker endpoints are equal to the maximum and minimum values. 7 subjects shown, 4 subjects s from cohort 1 (10 μg) and 3 subjects from cohort 2 (30 μg, denoted by black circles). Ex vivo=overnight ELISpot. Two subject timepoints were excluded at D180 due to positive COVID19 diagnosis (D108 & D110) and BNT162b2 vaccination (D137 & 135) prior to D180 timepoint.

FIG. 47 shows T cell responses against Nucleocapsid, Membrane, and ORF3a, as assessed by IFNγELISpot assay (post-IVS). SFU=Spot forming units per 10⁶ cells; Treatment day=day 1 single dose (10 μg or 30 μg) of GRT-R910 samRNA was administered. Boxes and horizontal bars denote interquartile range (IQR) and median values, respectively. Whisker endpoints are equal to the maximum and minimum values. 7 subjects shown, 4 subjects from cohort 1 (10 μg) and 3 subjects from cohort 2 (30 μg, denoted by black circles). IVS=in vitro expansion. Circles denote positive responses, triangles denote responses <LOD/<2×DMSO control. Two subject timepoints were excluded at D180 due to positive COVID19 diagnosis (D108 & D110) and BNT162b2 vaccination (D137 & 135) prior to D180 timepoint.

FIG. 48 shows T cell responses against Spike (top panel) and TCE epitopes (bottom panel) over a time course as a percentage of response to different T cell epitope pools.

FIG. 49 shows T cell responses as assessed by ELISpot against Spike overlapping peptides (left panel), TCE overlapping peptides (OLP, 15 amino acid peptides; middle panel), and minimal epitopes (8-11 amino acid peptides; right panel).

FIG. 50 shows T cell responses as assessed by ELISpot against Nucleocapsid, Membrane, and ORF3a as a percentage of response for both 10 μg (Cohort 1) and 30 μg (Cohort 2) doses.

FIG. 51 shows ex vivo ELISpot responses and MSD analysis of ELISpot supernatants for IFNγ, IL-2, and IL-4.

FIG. 52 shows intracellular cytokine staining for CD8+ T cells stimulated with TCE and ORF3a overlapping peptide pools and a ORF3a minimal epitope pool following a single 10 μg dose of samRNA. Shown is data for subject 0003 post treatment.

FIG. 53 shows intracellular cytokine staining for CD4+ T cells stimulated with TCE and ORF3a overlapping peptide pools and a ORF3a minimal epitope pool following a single 10 μg dose of samRNA. Shown is data for subject 0003 post treatment.

FIG. 54 shows post-expansion ELISpot responses in pre-pandemic donors to vaccine candidate GRT-R910 (SAM-SGP1-TCE5-SGP2-CTSpike_{G}F2P). Overlapping peptide pools (15mer) were used for expansion and ELISpot stimulation.

FIG. 55 shows ex vivo (left panel) and post-expansion (right panel) ELISpot responses to TCE5 components in convalescent donors for the indicated peptide pools.

FIG. 56 shows a schematic of vaccine candidates GRT-R912 (N-TCE11-Spike-beta; SEQ ID NO: 27981), GRT-R914 (TCE9-Spike-beta; SEQ ID NO: 27982), GRT-R918 (SAM-Nuc-TCE11-SpikeB.1.1.529 sequence; SEQ ID NO: 27976) and their antigenic coverage.

FIG. 57 shows a schematic of the cohorts for a clinical trial assessing vaccine candidates GRT-R912 (N-TCE11-Spike-beta), GRT-R914 (TCE9-Spike-beta), GRT-R918 (SAM-Nuc-TCE11-SpikeB.1.1.529 sequence).

FIG. 58 shows a summary of the participant demographics for the clinical trial (GO-012) assessing for a clinical trial assessing vaccine candidate GRT-R914 (TCE9-Spike-beta). *Cohort A1-A3: Participants were defined as “naïve” based on negative baseline N-Specific serology. **Cohort A4-A6: Participants were defined as “convalescent” if they confirmed a prior diagnosis of COVID-19 six months or more prior to screening or were positive on baseline N-Specific serology and did not have symptoms consistent with COVID-19 in the six months prior to screening.

FIG. 59A shows safety and reactogenicity summaries for vaccine candidate GRT-R914 (TCE9-Spike-beta) in “naïve” participants following dose 1.

FIG. 59B shows safety and reactogenicity summaries for vaccine candidate GRT-R914 (TCE9-Spike-beta) in “naïve” participants following dose 2.

FIG. 59C shows safety and reactogenicity summaries for vaccine candidate GRT-R914 (TCE9-Spike-beta) in “convalescent” participants following a single dose.

FIG. 60 shows Spike nAb against Beta variant following administration of GRT-R914 in COVID-naïve participants. Bottom rows of numbers represent negative N- & negative S-specific serology Middle rows of numbers represent negative N- & positive S-specific serology. Top rows of numbers represent negative N- & unknown S-specific serology subjects. nAb data were analyzed by microneutralization (MNA) assay. Note: Geometric mean in Cohort A1 does not include data of subject 105-0001 at Day 57 since 2^{nd} dose was not given due to an AE (Grade 1 neutropenia) and note that this subject had SARS-COV-2 infection on Day 58. Log₁₀ (ND₅₀ titer) is used for the y-axis scale and ND₅₀ titer is used for geometric means; Box plots with interquartile range and median are shown with the maximum and the minimum.

FIG. 61 shows Spike nAb against Delta variant following administration of GRT-R914 in COVID-naïve participants. Bottom rows of numbers represent negative N- & negative S-specific serology. Middle rows of numbers negative N- & positive S-specific serology. Top rows of numbers represent negative N- & unknown S-specific serology subjects. nAb data were analyzed by microneutralization (MNA) assay. Note: Geometric mean in Cohort A1 does not include data of subject 105-0001 at Day 57 since 2^{nd} dose was not given due to an AE (Grade 1 neutropenia) and note that this subject had SARS-COV-2 infection on Day 5. Log₁₀ (ND₅₀ titer) is used for the y-axis scale and ND₅₀ titer is used for geometric means; Box plots with interquartile range and median are shown with the maximum and the minimum.

FIG. 62 shows Spike nAb against Beta variant following administration of GRT-R914 in COVID-convalescent participants. nAb data were analyzed by microneutralization (MNA) assay. Log₁₀ (ND₅₀ titer) is used for the y-axis scale and ND₅₀ titer is used for geometric means; Box plots with interquartile range and median are shown with the maximum and the minimum.

FIG. 63 shows Spike nAb against Delta variant following administration of GRT-R914 in COVID-convalescent participants nAb data were analyzed by microneutralization (MNA) assay. Log₁₀ (ND₅₀ titer) is used for the y-axis scale and ND₅₀ titer is used for geometric means; Box plots with interquartile range and median are shown with the maximum and the minimum.

FIG. 64 shows Spike IgG following administration of GRT-R914 in COVID-naïve participants. Bottom rows of numbers represent Naïve & Negative S-specific subjects. Middle and top rows of numbers represent Naïve & Positive/NA S-specific subjects. Raw data is displayed in log₁₀ scale for presentation. Geometric means of raw data (ELU/mL) are presented. Geometric mean in Cohort A1 does not include subject 105-0001 at Day 57 since 2^{nd} dose received Grade 1 neutropenia and had SARS-COV-2 infection on Day 58.

FIG. 65 shows Spike IgG following administration of GRT-R914 in COVID-convalescent participants. Raw data is displayed in log₁₀ scale for presentation. Geometric means of raw data are presented.

FIG. 66A shows summaries of statistics for nAb data against Beta variant.

FIG. 66B shows summaries of statistics for fold change against Beta variant.

FIG. 66C shows summaries of statistics for nAb data against Delta variant.

FIG. 66D shows summaries of statistics for fold change against Delta variant.

FIG. 66E shows summaries of statistics for IgG data against wild-type.

FIG. 66F shows summaries of statistics for fold change against wild-type.

### DETAILED DESCRIPTION

### I. Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

As used herein the term “antigen” is a substance that stimulates an immune response. An antigen can be a neoantigen. An antigen can be a “shared antigen” that is an antigen found among a specific population, e.g., a specific population of SARS-CoV-2 patients with or at risk of infection for an infectious disease.

As used herein the term “antigen-based vaccine” is a vaccine composition based on one or more antigens, e.g., a plurality of antigens. The vaccines can be nucleotide-based (e.g., virally based, RNA based, or DNA based), protein-based (e.g., peptide based), or a combination thereof.

As used herein the term “candidate antigen” is a mutation or other aberration giving rise to a sequence that may represent an antigen.

As used herein the term “coding region” is the portion(s) of a gene that encode protein.

As used herein the term “coding mutation” is a mutation occurring in a coding region.

As used herein the term “ORF” means open reading frame.

As used herein the term “missense mutation” is a mutation causing a substitution from one amino acid to another.

As used herein the term “nonsense mutation” is a mutation causing a substitution from an amino acid to a stop codon or causing removal of a canonical start codon.

As used herein the term “frameshift mutation” is a mutation causing a change in the frame of the protein.

As used herein the term “indel” is an insertion or deletion of one or more nucleic acids.

As used herein, the term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Alternatively, sequence similarity or dissimilarity can be established by the combined presence or absence of particular nucleotides, or, for translated sequences, amino acids at selected sequence positions (e.g., sequence motifs).

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

As used herein the term “non-stop or read-through” is a mutation causing the removal of the natural stop codon.

As used herein the term “epitope” is the specific portion of an antigen typically bound by an antibody or T cell receptor.

As used herein the term “immunogenic” is the ability to stimulate an immune response, e.g., via T cells, B cells, or both.

As used herein the term “HLA binding affinity” “MHC binding affinity” means affinity of binding between a specific antigen and a specific MHC allele.

As used herein the term “bait” is a nucleic acid probe used to enrich a specific sequence of DNA or RNA from a sample.

As used herein the term “variant” is a difference between a subject's nucleic acids and the reference human genome used as a control.

As used herein the term “variant call” is an algorithmic determination of the presence of a variant, typically from sequencing.

As used herein the term “polymorphism” is a germline variant, i.e., a variant found in all DNA-bearing cells of an individual.

As used herein the term “somatic variant” is a variant arising in non-germline cells of an individual.

As used herein the term “allele” is a version of a gene or a version of a genetic sequence or a version of a protein.

As used herein the term “HLA type” is the complement of HLA gene alleles.

As used herein the term “nonsense-mediated decay” or “NMD” is a degradation of an mRNA by a cell due to a premature stop codon.

As used herein the term “exome” is a subset of the genome that codes for proteins. An exome can be the collective exons of a genome.

As used herein the term “logistic regression” is a regression model for binary data from statistics where the logit of the probability that the dependent variable is equal to one is modeled as a linear function of the dependent variables.

As used herein the term “neural network” is a machine learning model for classification or regression consisting of multiple layers of linear transformations followed by element-wise nonlinearities typically trained via stochastic gradient descent and back-propagation.

As used herein the term “proteome” is the set of all proteins expressed and/or translated by a cell, group of cells, or individual.

As used herein the term “peptidome” is the set of all peptides presented by MHC-I or MHC-II on the cell surface. The peptidome may refer to a property of a cell or a collection of cells (e.g., the infectious disease peptidome, meaning the union of the peptidomes of all cells that are infected by the infectious disease).

As used herein the term “ELISPOT” means Enzyme-linked immunosorbent spot assay—which is a common method for monitoring immune responses in humans and animals.

As used herein the term “dextramers” is a dextran-based peptide-MHC multimers used for antigen-specific T-cell staining in flow cytometry.

As used herein the term “tolerance or immune tolerance” is a state of immune non-responsiveness to one or more antigens, e.g. self-antigens.

As used herein the term “central tolerance” is a tolerance affected in the thymus, either by deleting self-reactive T-cell clones or by promoting self-reactive T-cell clones to differentiate into immunosuppressive regulatory T-cells (Tregs).

As used herein the term “peripheral tolerance” is a tolerance affected in the periphery by downregulating or energizing self-reactive T-cells that survive central tolerance or promoting these T cells to differentiate into Tregs.

The term “sample” can include a single cell or multiple cells or fragments of cells or an aliquot of body fluid, taken from a subject, by means including venipuncture, excretion, ejaculation, massage, biopsy, needle aspirate, lavage sample, scraping, surgical incision, or intervention or other means known in the art.

The term “subject” encompasses a cell, tissue, or organism, human or non-human, whether in vivo, ex vivo, or in vitro, male or female. The term subject is inclusive of mammals including humans.

The term “mammal” encompasses both humans and non-humans and includes but is not limited to humans, non-human primates, canines, felines, murines, bovines, equines, and porcines.

The term “clinical factor” refers to a measure of a condition of a subject, e.g., disease activity or severity. “Clinical factor” encompasses all markers of a subject's health status, including non-sample markers, and/or other characteristics of a subject, such as, without limitation, age and gender. A clinical factor can be a score, a value, or a set of values that can be obtained from evaluation of a sample (or population of samples) from a subject or a subject under a determined condition. A clinical factor can also be predicted by markers and/or other parameters such as gene expression surrogates. Clinical factors can include infection type (e.g., Coronavirus species), infection sub-type (e.g., SARS-CoV-2 variant), and medical history.

The term “antigen-encoding nucleic acid sequences derived from an infection” refers to nucleic acid sequences obtained from infected cells or an infectious disease organism, e.g. via RT-PCR; or sequence data obtained by sequencing the infected cell or infectious disease organism and then synthesizing the nucleic acid sequences using the sequencing data, e.g., via various synthetic or PCR-based methods known in the art. Derived sequences can include nucleic acid sequence variants, such as sequence-optimized nucleic acid sequence variants (e.g., codon-optimized and/or otherwise optimized for expression), that encode the same polypeptide sequence as the corresponding native infectious disease organism nucleic acid sequence. Derived sequences can include nucleic acid sequence variants that encode a modified infectious disease organism polypeptide sequence having one or more (e.g., 1, 2, 3, 4, or 5) mutations relative to a native infectious disease organism polypeptide sequence. For example, a modified polypeptide sequence can have one or more missense mutations relative to the native polypeptide sequence of an infectious disease organism protein.

The term “SARS-CoV-2 nucleic acid sequence encoding an immunogenic polypeptide” refers to nucleic acid sequences obtained from a SARS-CoV-2 virus, e.g. via RT-PCR; or sequence data obtained by sequencing a SARS-CoV-2 virus or a SARS-CoV-2 virus infected cell, and then synthesizing the nucleic acid sequences using the sequencing data, e.g., via various synthetic or PCR-based methods known in the art. Derived sequences can include nucleic acid sequence variants, such as sequence-optimized nucleic acid sequence variants (e.g., codon-optimized and/or otherwise optimized for expression), that encode the same polypeptide sequence as the corresponding native SARS-CoV-2 nucleic acid sequence. Derived sequences can include nucleic acid sequence variants that encode a modified SARS-CoV-2 polypeptide sequence having one or more (e.g., 1, 2, 3, 4, or 5) mutations relative to a native SARS-CoV-2 polypeptide sequence. For example, a modified Spike polypeptide sequence can have one or more mutations such as one or more missense mutations of R682, R815, K986P, or V987P relative to the native spike polypeptide sequence of a SARS-CoV-2 protein.

The term “alphavirus” refers to members of the family Togaviridae, and are positive-sense single-stranded RNA viruses. Alphaviruses are typically classified as either Old World, such as Sindbis, Ross River, Mayaro, Chikungunya, and Semliki Forest viruses, or New World, such as eastern equine encephalitis, Aura, Fort Morgan, or Venezuelan equine encephalitis and its derivative strain TC-83. Alphaviruses are typically self-replicating RNA viruses.

The term “alphavirus backbone” refers to minimal sequence(s) of an alphavirus that allow for self-replication of the viral genome. Minimal sequences can include conserved sequences for nonstructural protein-mediated amplification, a nonstructural protein 1 (nsP1) gene, a nsP2 gene, a nsP3 gene, a nsP4 gene, and a polyA sequence, as well as sequences for expression of subgenomic viral RNA including a subgenomic promoter (e.g., a 26S promoter element).

The term “sequences for nonstructural protein-mediated amplification” includes alphavirus conserved sequence elements (CSE) well known to those in the art. CSEs include, but are not limited to, an alphavirus 5′ UTR, a 51-nt CSE, a 24-nt CSE, a subgenomic promoter sequence (e.g., a 26S subgenomic promoter sequence), a 19-nt CSE, and an alphavirus 3′ UTR.

The term “RNA polymerase” includes polymerases that catalyze the production of RNA polynucleotides from a DNA template. RNA polymerases include, but are not limited to, bacteriophage derived polymerases including T3, T7, and SP6.

The term “lipid” includes hydrophobic and/or amphiphilic molecules. Lipids can be cationic, anionic, or neutral. Lipids can be synthetic or naturally derived, and in some instances biodegradable. Lipids can include cholesterol, phospholipids, lipid conjugates including, but not limited to, polyethyleneglycol (PEG) conjugates (PEGylated lipids), waxes, oils, glycerides, fats, and fat-soluble vitamins. Lipids can also include dilinoleylmethyl-4-dimethylaminobutyrate (MC3) and MC3-like molecules.

The term “lipid nanoparticle” or “LNP” includes vesicle like structures formed using a lipid containing membrane surrounding an aqueous interior, also referred to as liposomes. Lipid nanoparticles includes lipid-based compositions with a solid lipid core stabilized by a surfactant. The core lipids can be fatty acids, acylglycerols, waxes, and mixtures of these surfactants. Biological membrane lipids such as phospholipids, sphingomyelins, bile salts (sodium taurocholate), and sterols (cholesterol) can be utilized as stabilizers. Lipid nanoparticles can be formed using defined ratios of different lipid molecules, including, but not limited to, defined ratios of one or more cationic, anionic, or neutral lipids. Lipid nanoparticles can encapsulate molecules within an outer-membrane shell and subsequently can be contacted with target cells to deliver the encapsulated molecules to the host cell cytosol. Lipid nanoparticles can be modified or functionalized with non-lipid molecules, including on their surface. Lipid nanoparticles can be single-layered (unilamellar) or multi-layered (multilamellar). Lipid nanoparticles can be complexed with nucleic acid. Unilamellar lipid nanoparticles can be complexed with nucleic acid, wherein the nucleic acid is in the aqueous interior. Multilamellar lipid nanoparticles can be complexed with nucleic acid, wherein the nucleic acid is in the aqueous interior, or to form or sandwiched between

Abbreviations: MHC: major histocompatibility complex; HLA: human leukocyte antigen, or the human MHC gene locus; NGS: next-generation sequencing; PPV: positive predictive value; TSNA: tumor-specific neoantigen; FFPE: formalin-fixed, paraffin-embedded; NMD: nonsense-mediated decay; NSCLC: non-small-cell lung cancer; DC: dendritic cell.

It should be noted that, as used in the specification and the appended claims, the singular forms “a,” “an,” and “the” include plural referents unless the context clearly dictates otherwise.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

All references, issued patents and patent applications cited within the body of the specification are hereby incorporated by reference in their entirety, for all purposes.

### II. Antigen Identification

Research methods for NGS analysis of tumor and normal exome and transcriptomes have been described and applied in the antigen identification space. ^{6, 14, 15} Certain optimizations for greater sensitivity and specificity for antigen identification in the clinical setting can be considered. These optimizations can be grouped into two areas, those related to laboratory processes and those related to the NGS data analysis. The research methods described can also be applied to identification of antigens in other settings, such as identification of identifying antigens from an infectious disease organism (e.g., SARS-CoV-2), an infection in a subject, or an infected cell of a subject. Examples of optimizations are known to those skilled in the art, for example the methods described in more detail in U.S. Pat. No. 10,055,540, US Application Pub. No. US20200010849A1, international patent application publications WO/2018/195357 and WO/2018/208856, U.S. application Ser. No. 16/606,577, and international patent application PCT/US2020/021508, each herein incorporated by reference, in their entirety, for all purposes.

Methods for identifying antigens (e.g., antigens derived from an infectious disease organism) include identifying antigens that are likely to be presented on a cell surface (e.g., presented by MHC on an infected cell or an immune cell, including professional antigen presenting cells such as dendritic cells), and/or are likely to be immunogenic. As an example, one such method may comprise the steps of: obtaining at least one of exome, transcriptome or whole genome nucleotide sequencing and/or expression data from an infected cell or an infectious disease organism (e.g., SARS-CoV-2), wherein the nucleotide sequencing data and/or expression data is used to obtain data representing peptide sequences of each of a set of antigens (e.g., antigens derived from the infectious disease organism); inputting the peptide sequence of each antigen into one or more presentation models to generate a set of numerical likelihoods that each of the antigens is presented by one or more MHC alleles on a cell surface, such as an infected cell of the subject, the set of numerical likelihoods having been identified at least based on received mass spectrometry data; and selecting a subset of the set of antigens based on the set of numerical likelihoods to generate a set of selected antigens.

### IV. Antigens

Antigens can include nucleotides or polypeptides. For example, an antigen can be an RNA sequence that encodes for a polypeptide sequence. Antigens useful in vaccines can therefore include nucleotide sequences or polypeptide sequences.

Disclosed herein are peptides and nucleic acid sequences encoding peptides derived from any polypeptide associated with SARS-CoV-2, a SARS-CoV-2 infection in a subject, or a SARS-CoV-2 infected cell of a subject. Antigens can be derived from nucleotide sequences or polypeptide sequences of a SARS-CoV-2 virus. Polypeptide sequences of SARS-CoV-2 include, but are not limited to, predicted MHC class I epitopes shown in Table A, predicted MHC class II epitopes shown in Table B, predicted MHC class I epitopes shown in Table C, SARS-CoV-2 Spike peptides (e.g., peptides derived from SEQ ID NO:59), SARS-CoV-2 Membrane peptides (e.g., peptides derived from SEQ ID NO:61), SARS-CoV-2 Nucleocapsid peptides (e.g., peptides derived from SEQ ID NO:62), SARS-CoV-2 Envelope peptides (e.g., peptides derived from SEQ ID NO:63), SARS-CoV-2 replicase orf1a and orf1b peptides [such as one or more of non-structural proteins (nsp) 1-16], or any other peptide sequence encoded by a SARS-CoV-2 virus. Peptides and nucleic acid sequences encoding peptides can be derived from the Wuhan-Hu-1 SARS-CoV-2 isolate, sometimes referred to as the SARS-CoV-2 reference sequence (SEQ ID NO:76; NC_045512.2, herein incorporated by reference for all purposes). Peptides and nucleic acid sequences encoding peptides can be derived from an isolate distinct from the Wuhan-Hu-1 SARS-CoV-2 isolate, such as from the from a B.1.351 (“South African” or “Beta” or “501Y.V2”), a SARS-CoV-2 isolate the B.1.1.7 (“UK”) SARS-CoV-2 isolate, a B.1.1.529 (“Omicron”) isolate, or subisolates thereof, e.g., a B.1.1.529 BA5 subisolate. Peptides and nucleic acid sequences encoding peptides can be derived from an isolate distinct from the Wuhan-Hu-1 SARS-CoV-2 isolate, such as isolates having one or more mutations in proteins (also referred to as protein variants) with reference to the Wuhan-Hu-1 isolate. Vaccination strategies can include multiple vaccines with peptides and nucleic acid sequences encoding peptides derived from distinct isolates. For example, as an illustrative non-limiting example, a vaccine encoding a Spike protein from the Wuhan-Hu-1 SARS-CoV-2 isolate can be administered, followed by subsequent administration of a vaccine encoding a Spike protein from the B.1.351 (“South African” or “Beta” or “501Y.V2”) SARS-CoV-2 isolate (e.g., SEQ ID NO:112) or from the B.1.1.7 (“UK”) SARS-CoV-2 isolate (e.g., SEQ ID NO:110). The one or more variants can include, but are not limited to, mutations in the SARS-CoV-2 Spike protein, SARS-CoV-2 Membrane protein, SARS-CoV-2 Nucleocapsid protein, SARS-CoV-2 Envelope protein, SARS-CoV-2 replicase orf1a and orf1b protein [such as one or more of non-structural proteins (nsp) 1-16], or any other protein sequences encoded by a SARS-CoV-2 virus. Variants can be selected based on prevalence of the mutation among SARS-CoV-2 subtypes/isolates, such as mutations/variants that are present in 1% or greater, 2% or greater, 3% or greater, 4% or greater, 5% or greater, 6% or greater, 7% or greater, 8% or greater, 9% or greater, 10% or greater, 20% or greater, 30% or greater, 40% or greater, 50% or greater, 60% or greater, 70% or greater, 80% or greater, 90% or greater of SARS-CoV-2 subtypes/isolates. Examples of mutations in greater than 1% of isolates are shown in Table 1. Variants can be selected based on prevalence of the mutation among SARS-CoV-2 subtypes/isolates present in a specific population, such as a specific demographic or geographic population. An illustrative non-limiting example of a prevalent variant/mutation is the Spike D614G missense mutation found in 60.05% of genomes sequenced worldwide, and 70.46% and 58.49% of the sequences in Europe and North America, respectively. Accordingly, vaccines can be designed to encode at least one immunogenic polypeptide corresponding to a polypeptide encoded by a SARS-CoV-2 subtype the subject is infected with or at risk for infection by, such as for use in prophylactic vaccines for a specific demographic or geographic population at risk for infection by the specific SARS-CoV-2 subtype/isolate. Vaccines can be designed to encode at least one immunogenic polypeptide corresponding to a polypeptide encoded by SARS-CoV-2 and at least one immunogenic polypeptide corresponding to a polypeptide encoded by a Coronavirus species and/or sub-species other than SARS-CoV-2, e.g., the Severe acute respiratory syndrome (SARS) 2002-associated species (NC_004718.3, herein incorporated by reference for all purposes) and/or Middle East respiratory syndrome (MERS) 2012-associated species (NC_019843.3, herein incorporated by reference for all purposes). Vaccines can be designed to encode at least one immunogenic polypeptide corresponding to a polypeptide encoded by SARS-CoV-2 that is conserved (e.g., 100% amino acid sequence conservation between epitopes) between SARS-CoV-2 and a Coronavirus species and/or sub-species other than SARS-CoV-2, e.g., Severe acute respiratory syndrome (SARS) and/or Middle East respiratory syndrome (MERS) species. SARS-CoV-2 epitopes that are conserved between SARS-CoV-2 and a Coronavirus species and/or sub-species other than SARS-CoV-2 can include epitopes derived from a Coronavirus Spike protein, a Coronavirus Membrane protein, a Coronavirus Nucleocapsid protein, a Coronavirus Envelope protein, a Coronavirus replicase orf1a and orf1b protein [such as one or more of non-structural proteins (nsp) 1-16], or any other protein sequences encoded by a Coronavirus.

Antigens can be selected that are predicted to be presented on the cell surface of a cell, such as an infected cell or an immune cell, including professional antigen presenting cells such as dendritic cells. Antigens can be selected that are predicted to be immunogenic. Exemplary antigens predicted using the methods described herein to be presented on the cell surface by an MHC include predicted MHC class I epitopes shown in Table A, predicted MHC class II epitopes shown in Table B, and predicted MHC class I epitopes shown in Table C.

Antigens can be selected that have been validated to be presented by a specific HLA and/or stimulate an immune response, such as previously reported/validated in the literature (for example, as in Nelde et al. [Nature Immunology volume 22, pages 74-85 2021], Tarke et al. 2021, or Schelien et al. [bioRxiv 2020.08.13.249433]). The magnitude of stimulation of an immune response can be used to guide epitope/antigen selection, such as to select epitopes that stimulate as robust an immune response as possible, including when cassettes have a size constraint. As an illustrative non-limiting example of magnitude based-selection, the following can be used (1) An individual's magnitude is the sum of all epitope magnitudes across their respective diplotype alleles; (2) Each epitopes magnitude=(magnitude of response)×(Frequency of positive response/100), [e.g., using values found in Tarke et al. (Comprehensive analysis of T cell immunodominance and immunoprevalence of SARS-CoV-2 epitopes in COVID-19 cases. Cell Rep Med. 2021 Feb. 16; 2(2):100204. doi: 10.1016/j.xcrm.2021.100204. Epub 2021 Jan. 26.), herein incorporated by reference for all purposes]; (3) exclusion of epitopes other than those from starting proteins that span mutations with >5% frequency, optionally with mutations allowed in flanking regions; and/or (4) cassette order determined to minimize unintended junction epitopes across adjacent frames, as well as minimize consecutive frames in the same protein to reduce chance of functional protein fragments, as described herein.

A cassette can be constructed to encode one or more validated epitopes and/or at least 4, 5, 6, or 7 predicted epitopes, wherein at least 85%, 90%, or 95% of a population carries at least one HLA validated to present at least one of the one or more validated epitopes and/or at least one HLA predicted to present each of the at least 4, 5, 6, or 7 predicted epitopes. A cassette can be constructed to encode one or more validated epitopes and at least 4, 5, 6, or 7 predicted epitopes, wherein at least 85%, 90%, or 95% of a population carries at least one HLA validated to present at least one of the one or more validated epitopes or at least one HLA predicted to present each of the at least 4, 5, 6, or 7 predicted epitopes.

One or more polypeptides encoded by an antigen nucleotide sequence can comprise at least one of: a binding affinity with MHC with an IC50 value of less than 1000 nM, for MHC Class I peptides a length of 8-15, 8, 9, 10, 11, 12, 13, 14, or 15 amino acids, presence of sequence motifs within or near the peptide promoting proteasome cleavage, and presence or sequence motifs promoting TAP transport. For MHC Class II peptides a length 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the peptide promoting cleavage by extracellular or lysosomal proteases (e.g., cathepsins) or HLA-DM catalyzed HLA binding.

One or more antigens can be presented on the surface of an infected cell (e.g., a SARS-CoV-2 infected cell).

One or more antigens can be immunogenic in a subject having or suspected to have an infection (e.g., a SARS-CoV-2 infection), e.g., capable of stimulating a T cell response and/or a B cell response in the subject. One or more antigens can be immunogenic in a subject at risk of an infection (e.g., a SARS-CoV-2 infection), e.g., capable of stimulating a T cell response and/or a B cell response in the subject that provides immunological protection (i.e., immunity) against the infection, e.g., such as stimulating the production of memory T cells, memory B cells, or antibodies specific to the infection.

One or more antigens can be capable of stimulating a B cell response, such as the production of antibodies that recognize the one or more antigens (e.g., antibodies that recognize a SARS-CoV-2 antigen and/or virus). Antibodies can recognize linear polypeptide sequences or recognize secondary and tertiary structures. Accordingly, B cell antigens can include linear polypeptide sequences or polypeptides having secondary and tertiary structures, including, but not limited to, full-length proteins, protein subunits, protein domains, or any polypeptide sequence known or predicted to have secondary and tertiary structures. Antigens capable of stimulating a B cell response to an infection can be antigens found on the surface of an infectious disease organism (e.g., SARS-CoV-2). Antigens capable of stimulating a B cell response to an infection can be an intracellular antigen expressed in an infectious disease organism. SARS-CoV-2 antigens capable of stimulating a B cell response include, but are not limited to, SARS-CoV-2 Spike peptides, SARS-CoV-2 Membrane peptides, SARS-CoV-2 Nucleocapsid peptides, and SARS-CoV-2 Envelope peptides.

One or more antigens can include a combination of antigens capable of stimulating a T cell response (e.g., peptides including predicted T cell epitope sequences) and distinct antigens capable of stimulating a B cell response (e.g., full-length proteins, protein subunits, protein domains).

One or more antigens that stimulate an autoimmune response in a subject can be excluded from consideration in the context of vaccine generation for a subject.

The size of at least one antigenic peptide molecule (e.g., an epitope sequence) can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the antigenic peptide molecules are equal to or less than 50 amino acids.

Antigenic peptides and polypeptides can be: for MHC Class 115 residues or less in length and usually consist of between about 8 and about 11 residues, particularly 9 or 10 residues; for MHC Class II, 6-30 residues, inclusive.

If desirable, a longer peptide can be designed in several ways. In one case, when presentation likelihoods of peptides on HLA alleles are predicted or known, a longer peptide could consist of either: (1) individual presented peptides with an extensions of 2-5 amino acids toward the N- and C-terminus of each corresponding gene product; (2) a concatenation of some or all of the presented peptides with extended sequences for each. In another case, when sequencing reveals a long (>10 residues) epitope sequence present, a longer peptide would consist of: (3) the entire stretch of novel infectious disease-specific amino acids—thus bypassing the need for computational or in vitro test-based selection of the strongest HLA-presented shorter peptide. In both cases, use of a longer peptide allows endogenous processing by patient cells and may lead to more effective antigen presentation leading to increased T cell responses. Longer peptides can also include a full-length protein, a protein subunit, a protein domain, and combinations thereof of a peptide, such as those expressed in an infectious disease organism. Longer peptides (e.g., full-length protein, protein subunit, or protein domain) and combinations thereof can be included to stimulate a B cell response.

Antigenic peptides and polypeptides can be presented on an HLA protein. In some aspects antigenic peptides and polypeptides are presented on an HLA protein with greater affinity than a wild-type peptide. In some aspects, an antigenic peptide or polypeptide can have an IC50 of at least less than 5000 nM, at least less than 1000 nM, at least less than 500 nM, at least less than 250 nM, at least less than 200 nM, at least less than 150 nM, at least less than 100 nM, at least less than 50 nM or less.

In some aspects, antigenic peptides and polypeptides do not stimulate an autoimmune response and/or invoke immunological tolerance when administered to a subject.

Also provided are compositions comprising at least two or more antigenic peptides. In some embodiments the composition contains at least two distinct peptides. At least two distinct peptides can be derived from the same polypeptide. By distinct polypeptides is meant that the peptide vary by length, amino acid sequence, or both. The peptides can be derived from any polypeptide known to or suspected to be associated with an infectious disease organism, or peptides derived from any polypeptide known to or have been found to have altered expression in an infected cell in comparison to a normal cell or tissue (e.g., an infectious disease polynucleotide or polypeptide, including infectious disease polynucleotides or polypeptides with expression restricted to a host cell).

Antigenic peptides and polypeptides having a desired activity or property can be modified to provide certain desired attributes, e.g., improved pharmacological characteristics, while increasing or at least retaining substantially all of the biological activity of the unmodified peptide to bind the desired MHC molecule and activate the appropriate T cell. For instance, antigenic peptide and polypeptides can be subject to various changes, such as substitutions, either conservative or non-conservative, where such changes might provide for certain advantages in their use, such as improved MHC binding, stability or presentation. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications can be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

Modifications of peptides and polypeptides with various amino acid mimetics or unnatural amino acids can be particularly useful in increasing the stability of the peptide and polypeptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half-life of the peptides can be conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4 degrees C.) for 15 minutes and then spun to pellet the precipitated serum proteins. The presence of the peptides is then determined by reversed-phase HPLC using stability-specific chromatography conditions.

The peptides and polypeptides can be modified to provide desired attributes other than improved serum half-life. For instance, the ability of the peptides to stimulate CTL activity can be enhanced by linkage to a sequence which contains at least one epitope that is capable of inducing a T helper cell response. Immunogenic peptides/T helper conjugates can be linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus can be a hetero- or homo-oligomer. When present, the spacer will usually be at least one or two residues, more usually three to six residues. Alternatively, the peptide can be linked to the T helper peptide without a spacer.

Polypeptides encoding antigens can be modified to alter processing of the polypeptides, such as protease cleavage and/or other post-translation processing. Polypeptides encoding antigens can be modified such that the antigen favors a specific conformation. Polypeptides encoding antigens can be modified such that the mutations (e.g., one or more missense mutations) disrupt a specific conformation in the antigen, such as through the introduction of prolines that disrupt secondary and tertiary structures (e.g., alpha-helix or beta-sheet formation). Altering, reducing, or eliminating processing or conformation changes may, in some instances, bias the antigen to favor states favorable to neutralizing antibody production. In a series of illustrative examples, SARS-CoV-2 Spike mutations at amino acids 682, 815, 987, and 988 are engineered to bias the Spike protein to remain in a predominantly prefusion state, a potentially preferable state for antibody-mediated neutralization. Specifically, without wishing to be bound by theory, mutations at R682 (e.g., R682V) disrupt the Furin cleavage site involved in processing Spike into S1 and S2; mutations at R815 (e.g., R815N) disrupt the cleavage site within S2; and mutations at K986 and V987, such as K986P and V987P introducing two prolines, that interfere with the secondary structure of Spike making it less likely to be processed from the pre to post fusion state. Accordingly, an antigen cassette can encode a modified Spike protein having at least one mutation selected from: a Spike R682V mutation, a Spike R815N mutation, a Spike K986P mutation, a Spike V987P mutation, and combinations thereof with reference the Wuhan-Hu-1 isolate (see SEQ ID NO:59 reference and SEQ ID NO:60/SEQ ID NO:90 containing mutations). Modified polypeptide sequences can be at least 60%, 70%, 80%, or 90% identical to a native SARS-CoV-2 polypeptide sequence. Modified polypeptide sequences can be at least 91%, 92%, 93%, or 94% identical to a native SARS-CoV-2 polypeptide sequence. Modified polypeptide sequences can be at least 95%, 96%, 97%, 98%, or 99% identical to a native SARS-CoV-2 polypeptide sequence. Modified polypeptide sequences can be at least 99.1%, 99.2%, 99.3%, 99.4%, 99.5%, 99.6%, 99.7%, 99.8%, or 99.9% identical to a native SARS-CoV-2 polypeptide sequence.

An antigenic peptide can be linked to the T helper peptide either directly or via a spacer either at the amino or carboxy terminus of the peptide. The amino terminus of either the antigenic peptide or the T helper peptide can be acylated. Exemplary T helper peptides include tetanus toxoid 830-843, influenza 307-319, malaria circumsporozoite 382-398 and 378-389.

Proteins or peptides can be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and can be found at computerized databases known to those of ordinary skill in the art. One such database is the National Center for Biotechnology Information's Genbank and GenPept databases located at the National Institutes of Health website. The coding regions for known genes can be amplified and/or expressed using the techniques disclosed herein or as would be known to those of ordinary skill in the art. Alternatively, various commercial preparations of proteins, polypeptides and peptides are known to those of skill in the art.

In a further aspect, an antigen includes a nucleic acid (e.g. polynucleotide) that encodes an antigenic peptide or portion thereof. The polynucleotide can be, e.g., DNA, cDNA, PNA, CNA, RNA (e.g., mRNA), either single- and/or double-stranded, or native or stabilized forms of polynucleotides, such as, e.g., polynucleotides with a phosphorothioate backbone, or combinations thereof and it may or may not contain introns. A polynucleotide sequence encoding an antigen can be sequence-optimized to improve expression, such as through improving transcription, translation, post-transcriptional processing, and/or RNA stability. For example, polynucleotide sequence encoding an antigen can be codon-optimized. “Codon-optimization” herein refers to replacing infrequently used codons, with respect to codon bias of a given organism, with frequently used synonymous codons. Polynucleotide sequences can be optimized to improve post-transcriptional processing, for example optimized to reduce unintended splicing, such as through removal of splicing motifs (e.g., canonical and/or cryptic/non-canonical splice donor, branch, and/or acceptor sequences) and/or introduction of exogenous splicing motifs (e.g., splice donor, branch, and/or acceptor sequences) to bias favored splicing events. Exogenous intron sequences include, but are not limited to, those derived from SV40 (e.g., an SV40 mini-intron [SEQ ID NO:88]) and derived from immunoglobulins (e.g., human β-globin gene). Exogenous intron sequences can be incorporated between a promoter/enhancer sequence and the antigen(s) sequence. Exogenous intron sequences for use in expression vectors are described in more detail in Callendret et al. (Virology. 2007 Jul. 5; 363(2): 288-302), herein incorporated by reference for all purposes. Polynucleotide sequences can be optimized to improve transcript stability, for example through removal of RNA instability motifs (e.g., AU-rich elements and 3′ UTR motifs) and/or repetitive nucleotide sequences. Polynucleotide sequences can be optimized to improve accurate transcription, for example through removal of cryptic transcriptional initiators and/or terminators. Polynucleotide sequences can be optimized to improve translation and translational accuracy, for example through removal of cryptic AUG start codons, premature polyA sequences, and/or secondary structure motifs. Polynucleotide sequences can be optimized to improve nuclear export of transcripts, such as through addition of a Constitutive Transport Element (CTE), RNA Transport Element (RTE), or Woodchuck Posttranscriptional Regulatory Element (WPRE). Nuclear export signals for use in expression vectors are described in more detail in Callendret et al. (Virology. 2007 Jul. 5; 363(2): 288-302), herein incorporated by reference for all purposes. Polynucleotide sequences can be optimized with respect to GC content, for example to reflect the average GC content of a given organism. Sequence optimization can balance one or more sequence properties, such as transcription, translation, post-transcriptional processing, and/or RNA stability. Sequence optimization can generate an optimal sequence balancing each of transcription, translation, post-transcriptional processing, and RNA stability. Sequence optimization algorithms are known to those of skill in the art, such as GeneArt (Thermo Fisher), Codon Optimization Tool (IDT), Cool Tool, SGI-DNA (La Jolla California). One or more regions of an antigen-encoding protein can be sequence-optimized separately. As a non-limiting illustrative example, SARS-CoV-2 Spike protein can be sequence-optimized (or unoptimized) in the S1 region of the protein and the S2 region is separately optimized (e.g., optimized using a different algorithm and/or optimized for one or more sequence properties specific for the S2 region).

A method disclosed herein can also include identifying one or more T cells that are antigen-specific for at least one of the antigens in the subset. In some embodiments, the identification comprises co-culturing the one or more T cells with one or more of the antigens in the subset under conditions that expand the one or more antigen-specific T cells. In further embodiments, the identification comprises contacting the one or more T cells with a tetramer comprising one or more of the antigens in the subset under conditions that allow binding between the T cell and the tetramer. In even further embodiments, the method disclosed herein can also include identifying one or more T cell receptors (TCR) of the one or more identified T cells. In certain embodiments, identifying the one or more T cell receptors comprises sequencing the T cell receptor sequences of the one or more identified T cells. The method disclosed herein can further comprise genetically engineering a plurality of T cells to express at least one of the one or more identified T cell receptors; culturing the plurality of T cells under conditions that expand the plurality of T cells; and infusing the expanded T cells into the subject. In some embodiments, genetically engineering the plurality of T cells to express at least one of the one or more identified T cell receptors comprises cloning the T cell receptor sequences of the one or more identified T cells into an expression vector; and transfecting each of the plurality of T cells with the expression vector. In some embodiments, the method disclosed herein further comprises culturing the one or more identified T cells under conditions that expand the one or more identified T cells; and infusing the expanded T cells into the subject.

Also disclosed herein is an isolated T cell that is antigen-specific for at least one selected antigen in the subset.

A still further aspect provides an expression vector capable of expressing a polypeptide or portion thereof. Expression vectors for different cell types are well known in the art and can be selected without undue experimentation. Generally, DNA is inserted into an expression vector, such as a plasmid, in proper orientation and correct reading frame for expression. If necessary, DNA can be linked to the appropriate transcriptional and translational regulatory control nucleotide sequences recognized by the desired host, although such controls are generally available in the expression vector. The vector is then introduced into the host through standard techniques. Guidance can be found e.g. in Sambrook et al. (1989) Molecular Cloning, A Laboratory Manual, Cold Spring Harbor Laboratory, Cold Spring Harbor, N.Y.

### V. Vaccine Compositions

Also disclosed herein is an immunogenic composition, e.g., a vaccine composition, capable of raising a specific immune response, e.g., an infectious disease organism-specific immune response. Vaccine compositions typically comprise one or a plurality of antigens, e.g., selected using a method described herein. Vaccine compositions can also be referred to as vaccines.

A vaccine can contain between 1 and 30 peptides, 2, 3, 4, 5, 6, 7, 8, 9, 10, 11, 12, 13, 14, 15, 16, 17, 18, 19, 20, 21, 22, 23, 24, 25, 26, 27, 28, 29, or 30 different peptides, 6, 7, 8, 9, 10 11, 12, 13, or 14 different peptides, or 12, 13 or 14 different peptides. Peptides can include post-translational modifications. A vaccine can contain between 1 and 100 or more nucleotide sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different nucleotide sequences, 6, 7, 8, 9, 10 11, 12, 13, or 14 different nucleotide sequences, or 12, 13 or 14 different nucleotide sequences. A vaccine can contain between 1 and 30 antigen sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different antigen sequences, 6, 7, 8, 9, 10 11, 12, 13, or 14 different antigen sequences, or 12, 13 or 14 different antigen sequences.

A vaccine can contain between 1 and 30 antigen-encoding nucleic acid sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different antigen-encoding nucleic acid sequences, 6, 7, 8, 9, 10 11, 12, 13, or 14 different antigen-encoding nucleic acid sequences, or 12, 13 or 14 different antigen-encoding nucleic acid sequences. Antigen-encoding nucleic acid sequences can refer to the antigen encoding portion of an “antigen cassette.” Features of an antigen cassette are described in greater detail herein. An antigen-encoding nucleic acid sequence can contain one or more epitope-encoding nucleic acid sequences (e.g., an antigen-encoding nucleic acid sequence encoding concatenated T cell epitopes).

A vaccine can contain between 1 and 30 distinct epitope-encoding nucleic acid sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more distinct epitope-encoding nucleic acid sequences, 6, 7, 8, 9, 10 11, 12, 13, or 14 distinct epitope-encoding nucleic acid sequences, or 12, 13 or 14 distinct epitope-encoding nucleic acid sequences. Epitope-encoding nucleic acid sequences can refer to sequences for individual epitope sequences, such as each of the T cell epitopes in an antigen-encoding nucleic acid sequence encoding concatenated T cell epitopes.

A vaccine can contain at least two repeats of an epitope-encoding nucleic acid sequence. A used herein, a “repeat” refers to two or more iterations of an identical nucleic acid epitope-encoding nucleic acid sequence (inclusive of the optional 5′ linker sequence and/or the optional 3′ linker sequences described herein) within an antigen-encoding nucleic acid sequence. In one example, the antigen-encoding nucleic acid sequence portion of a cassette encodes at least two repeats of an epitope-encoding nucleic acid sequence. In further non-limiting examples, the antigen-encoding nucleic acid sequence portion of a cassette encodes more than one distinct epitope, and at least one of the distinct epitopes is encoded by at least two repeats of the nucleic acid sequence encoding the distinct epitope (i.e., at least two distinct epitope-encoding nucleic acid sequences). In illustrative non-limiting examples, an antigen-encoding nucleic acid sequence encodes epitopes A, B, and C encoded by epitope-encoding nucleic acid sequences epitope-encoding sequence A (E_{A}), epitope-encoding sequence B (E_{B}), and epitope-encoding sequence C (E_{C}), and exemplary antigen-encoding nucleic acid sequences having repeats of at least one of the distinct epitopes are illustrated by, but is not limited to, the formulas below:
Repeat of one distinct epitope (repeat of epitope A):

E_{A}-E_{B}-E_{C}-E_{A}; or

E_{A}-E_{A}-E_{B}-E_{C}
Repeat of multiple distinct epitopes (repeats of epitopes A, B, and C):

E_{A}-E_{B}-E_{C}-E_{A}-E_{B}-E_{C}; or

E_{A}-E_{A}-E_{B}-E_{B}-E_{C}-E_{C}
Multiple repeats of multiple distinct epitopes (repeats of epitopes A, B, and C):

E_{A}-E_{B}-E_{C}-E_{A}-E_{B}-E_{C}-E_{A}-E_{B}-E_{C}; or

E_{A}-E_{A}-E_{A}-E_{B}-E_{B}-E_{B}-E_{C}-E_{C}-E_{C}

The above examples are not limiting and the antigen-encoding nucleic acid sequences having repeats of at least one of the distinct epitopes can encode each of the distinct epitopes in any order or frequency. For example, the order and frequency can be a random arrangement of the distinct epitopes, e.g., in an example with epitopes A, B, and C, by the formula E_{A}-E_{B}-E_{C}-E_{C}-E_{A}-E_{B}-E_{A}-E_{C}-E_{A}-E_{C}-E_{C}-E_{B}.

Also provided for herein is an antigen-encoding cassette, the antigen-encoding cassette having at least one antigen-encoding nucleic acid sequence described, from 5′ to 3′, by the formula:

(Eₓ-(E^{N}ₐ)_{y})_{z}
where E represents a nucleotide sequence comprising at least one of the at least one distinct epitope-encoding nucleic acid sequences,
n represents the number of separate distinct epitope-encoding nucleic acid sequences and is any integer including 0,
E^{N} represents a nucleotide sequence comprising the separate distinct epitope-encoding nucleic acid sequence for each corresponding n,
for each iteration of z: x=0 or 1, y=0 or 1 for each n, and at least one of x or y=1, and z=2 or greater, wherein the antigen-encoding nucleic acid sequence comprises at least two iterations of E, a given E^{N}, or a combination thereof.

Each E or E^{N} can independently comprise any epitope-encoding nucleic acid sequence described herein (e.g., a nucleotide sequence encoding a polypeptide sequence as set forth in Table A, Table B, and/or Table C). For example, Each E or E^{N} can independently comprises a nucleotide sequence described, from 5′ to 3′, by the formula (L5_{b}-N_{c}-L3_{d}), where N comprises the distinct epitope-encoding nucleic acid sequence associated with each E or E^{N}, where c=1, L⁵ comprises a 5′ linker sequence, where b=0 or 1, and L3 comprises a 3′ linker sequence, where d=0 or 1. Epitopes and linkers that can be used are further described herein.

Repeats of an epitope-encoding nucleic acid sequences (inclusive of optional 5′ linker sequence and/or the optional 3′ linker sequences) can be linearly linked directly to one another (e.g., E_{A}-E_{A}- . . . as illustrated above). Repeats of an epitope-encoding nucleic acid sequences can be separated by one or more additional nucleotides sequences. In general, repeats of an epitope-encoding nucleic acid sequences can be separated by any size nucleotide sequence applicable for the compositions described herein. In one example, repeats of an epitope-encoding nucleic acid sequences can be separated by a separate distinct epitope-encoding nucleic acid sequence (e.g., E_{A}-E_{B}-E_{C}-E_{A} . . . , as illustrated above). In examples where repeats are separated by a single separate distinct epitope-encoding nucleic acid sequence, and each epitope-encoding nucleic acid sequences (inclusive of optional 5′ linker sequence and/or the optional 3′ linker sequences) encodes a peptide 25 amino acids in length, the repeats can be separated by 75 nucleotides, such as in antigen-encoding nucleic acid represented by E_{A}-E_{B}-E_{A} . . . , E_{A} is separated by 75 nucleotides. In an illustrative example, an antigen-encoding nucleic acid having the sequence VTNTEMFVTAPDNLGYMYEVQWPGQTQPQIANCSVYDFFVWLHYYSVRDTVTNTEMF VTAPDNLGYMYEVQWPGQTQPQIANCSVYDFFVWLHYYSVRDT (SEQ ID NO: 115) encoding repeats of 25mer antigens Trp1 (VTNTEMFVTAPDNLGYMYEVQWPGQ; SEQ ID NO: 116) and Trp2 (TQPQIANCSVYDFFVWLHYYSVRDT; SEQ ID NO: 117), the repeats of Trp1 are separated by the 25mer Trp2 and thus the repeats of the Trp1 epitope-encoding nucleic acid sequences are separated the 75 nucleotide Trp2 epitope-encoding nucleic acid sequence. In examples where repeats are separated by 2, 3, 4, 5, 6, 7, 8, or 9 separate distinct epitope-encoding nucleic acid sequence, and each epitope-encoding nucleic acid sequences (inclusive of optional 5′ linker sequence and/or the optional 3′ linker sequences) encodes a peptide 25 amino acids in length, the repeats can be separated by 150, 225, 300, 375, 450, 525, 600, or 675 nucleotides, respectively.

In one embodiment, different peptides and/or polypeptides or nucleotide sequences encoding them are selected so that the peptides and/or polypeptides capable of associating with different MHC molecules, such as different MHC class I molecules and/or different MHC class II molecules. In some aspects, one vaccine composition comprises coding sequence for peptides and/or polypeptides capable of associating with the most frequently occurring MHC class I molecules and/or different MHC class II molecules. Hence, vaccine compositions can comprise different fragments capable of associating with at least 2 preferred, at least 3 preferred, or at least 4 preferred MHC class I molecules and/or different MHC class II molecules.

The vaccine composition can stimulate a specific cytotoxic T-cell response and a specific helper T-cell response.

The vaccine composition can stimulate a specific B-cell response (e.g., an antibody response).

The vaccine composition can stimulate a specific cytotoxic T-cell response, a specific helper T-cell response, and/or a specific B-cell response. The vaccine composition can stimulate a specific cytotoxic T-cell response and a specific B-cell response. The vaccine composition can stimulate a specific helper T-cell response and a specific B-cell response. The vaccine composition can stimulate a specific cytotoxic T-cell response, a specific helper T-cell response, and a specific B-cell response.

A combination of vaccine compositions can stimulate a specific cytotoxic T-cell response, a specific helper T-cell response, and/or a specific B-cell response. Vaccine compositions can be homologous and stimulate a specific cytotoxic T-cell response, a specific helper T-cell response, and/or a specific B-cell response in combination. Vaccine compositions can be homologous and stimulate a specific cytotoxic T-cell response, a specific helper T-cell response, and a specific B-cell response in combination. Vaccine compositions can be heterologous and stimulate a specific cytotoxic T-cell response, a specific helper T-cell response, and/or a specific B-cell response in combination. Vaccine compositions can be heterologous and stimulate a specific cytotoxic T-cell response, a specific helper T-cell response, and a specific B-cell response in combination. Heterologous vaccines include an identical antigen cassette encoded by different vaccine platforms, e.g., a viral vaccine (e.g., a ChAdV-based platform) and a mRNA vaccine (e.g., a SAM-based platform). Heterologous vaccines include different antigen cassettes (e.g., a Spike cassette and a separate T cell epitope encoding cassette, or epitopes/antigens derived from different subtype isolates of SARS-CoV-2, such as Spike protein variants from a Wuhan-Hu-1 subtype isolate and a B.1.351 subtype isolate) encoded by the same vaccine platform, e.g., either a viral vaccine (e.g., a ChAdV-based platform) or a mRNA vaccine (e.g., a SAM-based platform). Heterologous vaccines include different antigen cassettes (e.g., a Spike cassette and a separate T cell epitope encoding cassette or epitopes/antigens derived from different isolate/subtype of SARS-CoV-2, such as Spike protein variants from a Wuhan-Hu-1 subtype isolate and a B.1.351 subtype isolate) encoded by different vaccine platforms, e.g., a viral vaccine (e.g., a ChAdV-based platform) and a mRNA vaccine (e.g., a SAM-based platform). For example, as an illustrative non-limiting example, a viral vaccine (e.g., a ChAdV-based platform) can in particular stimulate a robust cytotoxic T-cell response and a mRNA vaccine (e.g., a SAM-based platform) can in particular stimulate a robust B-cell response.

A vaccine composition can further comprise an adjuvant and/or a carrier. Examples of useful adjuvants and carriers are given herein below. A composition can be associated with a carrier such as e.g. a protein or an antigen-presenting cell such as, e.g., a dendritic cell (DC) capable of presenting the peptide to a T-cell.

Adjuvants are any substance whose admixture into a vaccine composition increases or otherwise modifies the immune response to an antigen. Carriers can be scaffold structures, for example a polypeptide or a polysaccharide, to which an antigen, is capable of being associated. Optionally, adjuvants are conjugated covalently or non-covalently.

The ability of an adjuvant to increase an immune response to an antigen is typically manifested by a significant or substantial increase in an immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th response into a primarily cellular, or Th response.

Suitable adjuvants include, but are not limited to 1018 ISS, alum, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-E_{C}, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are useful. Several immunological adjuvants (e.g., MF59) specific for dendritic cells and their preparation have been described previously (Dupuis M, et al., Cell Immunol. 1998; 186(1):18-27; Allison A C; Dev Biol Stand. 1998; 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

CpG immunostimulatory oligonucleotides have also been reported to enhance the effects of adjuvants in a vaccine setting. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C) (e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives can readily be determined by the skilled artisan without undue experimentation. Additional adjuvants include colony-stimulating factors, such as Granulocyte Macrophage Colony Stimulating Factor (GM-CSF, sargramostim).

A vaccine composition can comprise more than one different adjuvant. Furthermore, a therapeutic composition can comprise any adjuvant substance including any of the above or combinations thereof. It is also contemplated that a vaccine and an adjuvant can be administered together or separately in any appropriate sequence.

A carrier (or excipient) can be present independently of an adjuvant. The function of a carrier can for example be to increase the molecular weight of in particular mutant to increase activity or immunogenicity, to confer stability, to increase the biological activity, or to increase serum half-life. Furthermore, a carrier can aid presenting peptides to T-cells. A carrier can be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. However, tetanus toxoid and/or diphtheria toxoid are suitable carriers. Alternatively, the carrier can be dextrans for example Sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is possible if a trimeric complex of peptide antigen, MHC molecule, and APC is present. Correspondingly, it may enhance the immune response if not only the peptide is used for activation of CTLs, but if additionally APCs with the respective MHC molecule are added. Therefore, in some embodiments a vaccine composition additionally contains at least one antigen presenting cell.

Antigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more antigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291):1337-41, Lu et al., Efficient identification of mutated cancer antigens recognized by T cells associated with durable tumor regressions, Clin Cancer Res. (2014) 20(13):3401-10). Upon introduction into a host, infected cells express the antigens, and thereby stimulate a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of antigens, e.g., Salmonella typhi vectors, and the like will be apparent to those skilled in the art from the description herein.

### V.A. Antigen Cassette

The methods employed for the selection of one or more antigens, the cloning and construction of a “cassette” and its insertion into a viral vector are within the skill in the art given the teachings provided herein. By “antigen cassette” or “cassette” is meant the combination of a selected antigen or plurality of antigens (e.g., antigen-encoding nucleic acid sequences) and the other regulatory elements necessary to transcribe the antigen(s) and express the transcribed product. The selected antigen or plurality of antigens can refer to distinct epitope sequences, e.g., an antigen-encoding nucleic acid sequence in the cassette can encode an epitope-encoding nucleic acid sequence (or plurality of epitope-encoding nucleic acid sequences) such that the epitopes are transcribed and expressed. An antigen or plurality of antigens can be operatively linked to regulatory components in a manner which permits transcription. Such components include conventional regulatory elements that can drive expression of the antigen(s) in a cell transfected with the viral vector. Thus the antigen cassette can also contain a selected promoter which is linked to the antigen(s) and located, with other, optional regulatory elements, within the selected viral sequences of the recombinant vector. A cassette can have one or more antigen-encoding nucleic acid sequences, such as a cassette containing multiple antigen-encoding nucleic acid sequences each independently operably linked to separate promoters and/or linked together using other multicistonic systems, such as 2A ribosome skipping sequence elements (e.g., E2A, P2A, F2A, or T2A sequences) or Internal Ribosome Entry Site (IRES) sequence elements. A linker can also have a cleavage site, such as a TEV or furin cleavage site. Linkers with cleavage sites can be used in combination with other elements, such as those in a multicistronic system. In a non-limiting illustrative example, a furin protease cleavage site can be used in conjuction with a 2A ribosome skipping sequence element such that the furin protease cleavage site is configured to facilitate removal of the 2A sequence following translation. In a cassette containing more than one antigen-encoding nucleic acid sequences, each antigen-encoding nucleic acid sequence can contain one or more epitope-encoding nucleic acid sequences (e.g., an antigen-encoding nucleic acid sequence encoding concatenated T cell epitopes). In illustrative examples of multicistronic formats, cassettes encoding SARS-CoV-2 antigens are configured as follows: (1) endogenous 26S promoter-Spike protein-T2A-Membrane protein, or (2) endogenous 26S promoter-Spike protein-26S promoter-concatenated T cell epitopes.

Useful promoters can be constitutive promoters or regulated (inducible) promoters, which will enable control of the amount of antigen(s) to be expressed. For example, a desirable promoter is that of the cytomegalovirus immediate early promoter/enhancer [see, e.g., Boshart et al, Cell, 41:521-530 (1985)]. Another desirable promoter includes the Rous sarcoma virus LTR promoter/enhancer. Still another promoter/enhancer sequence is the chicken cytoplasmic beta-actin promoter [T. A. Kost et al, Nucl. Acids Res., 11(23):8287 (1983)]. Other suitable or desirable promoters can be selected by one of skill in the art.

The antigen cassette can also include nucleic acid sequences heterologous to the viral vector sequences including sequences providing signals for efficient polyadenylation of the transcript (poly(A), poly-A or pA) and introns with functional splice donor and acceptor sites. A common poly-A sequence which is employed in the exemplary vectors of this invention is that derived from the papovavirus SV-40. The poly-A sequence generally can be inserted in the cassette following the antigen-based sequences and before the viral vector sequences. A common intron sequence can also be derived from SV-40, and is referred to as the SV-40 T intron sequence. An antigen cassette can also contain such an intron, located between the promoter/enhancer sequence and the antigen(s). Selection of these and other common vector elements are conventional [see, e.g., Sambrook et al, “Molecular Cloning. A Laboratory Manual.”, 2d edit., Cold Spring Harbor Laboratory, New York (1989) and references cited therein] and many such sequences are available from commercial and industrial sources as well as from Genbank.

An antigen cassette can have one or more antigens. For example, a given cassette can include 1-10, 1-20, 1-30, 10-20, 15-25, 15-20, 1, 2, 3, 4, 5, 6, 7, 8, 9, 10, 11, 12, 13, 14, 15, 16, 17, 18, 19, 20, or more antigens. Antigens can be linked directly to one another. Antigens can also be linked to one another with linkers. Antigens can be in any orientation relative to one another including N to C or C to N.

As described elsewhere, the antigen cassette can be located in the site of any selected deletion in the viral vector backbone, such as the site of the E1 gene region deletion or E3 gene region deletion of a ChAd-based vector or the deleted structural proteins of a VEE backbone, among others which may be selected.

The antigen encoding sequence (e.g., cassette or one or more of the nucleic acid sequences encoding an immunogenic polypeptide in the cassette) can be described using the following formula to describe the ordered sequence of each element, from 5′ to 3′:

Pₐ-(L5_{b}-N_{c}-L3_{d})_{X}-(G5ₑ-U_{f})_{Y}-G3_{g}

wherein P comprises the second promoter nucleotide sequence, where a=0 or 1, where c=1, N comprises one of the SARS-CoV-2 derived nucleic acid sequences described herein, optionally wherein each N encodes a polypeptide sequence as set forth in Table A, Table B, and/or Table C,L5 comprises the 5′ linker sequence, where b=0 or 1, L³ comprises the 3′ linker sequence, where d=0 or 1, G5 comprises one of the at least one nucleic acid sequences encoding a GPGPG amino acid linker (SEQ ID NO: 56), where e=0 or 1, G3 comprises one of the at least one nucleic acid sequences encoding a GPGPG amino acid linker (SEQ ID NO: 56), where g=0 or 1, U comprises one of the at least one MHC class II epitope-encoding nucleic acid sequence, where f=1, X=1 to 400, where for each X the corresponding N_{c} is a SARS-CoV-2 derived nucleic acid sequence, and Y=0, 1, or 2, where for each Y the corresponding U_{f} is a (1) universal MHC class II epitope-encoding nucleic acid sequence, optionally wherein the at least one universal sequence comprises at least one of Tetanus toxoid and PADRE, or (2) a MHC class II SARS-CoV-2 derived epitope-encoding nucleic acid sequence. In some aspects, for each X the corresponding N_{c} is a distinct SARS-CoV-2 derived nucleic acid sequence. In some aspects, for each Y the corresponding U_{f} is a distinct universal MHC class II epitope-encoding nucleic acid sequence or a distinct MHC class II SARS-CoV-2 derived epitope-encoding nucleic acid sequence. The above antigen encoding sequence formula in some instances only describes the portion of an antigen cassette encoding concatenated epitope sequences, such as concatenated T cell epitopes. For example, in cassettes encoding concatenated T cell epitopes and one or more full-length SARS-CoV-2 proteins, the above antigen encoding sequence formula describes the concatenated T cell epitopes and separately the cassette encodes one or more full-length SARS-CoV-2 proteins that are linked optionally using a multicistonic system, such as 2A ribosome skipping sequence elements (e.g., E2A, P2A, F2A, or T2A sequences), a Internal Ribosome Entry Site (IRES) sequence elements, and/or independently operably linked to a separate promoter.

In one example, elements present include where b=1, d=1, e=1, g=1, h=1, X=18, Y=2, and the vector backbone comprises a ChAdV68 vector, a=1, P is a CMV promoter, the at least one second poly(A) sequence is present, wherein the second poly(A) sequence is an exogenous poly(A) sequence to the vector backbone, and optionally wherein the exogenous poly(A) sequence comprises an SV40 poly(A) signal sequence or a BGH poly(A) signal sequence, and each N encodes a MHC class I epitope 7-15 amino acids in length, a MHC class II epitope, an epitope capable of stimulating a B cell response, or combinations thereof, L5 is a native 5′ linker sequence that encodes a native N-terminal amino acid sequence of the epitope, and wherein the 5′ linker sequence encodes a peptide that is at least 3 amino acids in length, L3 is a native 3′ linker sequence that encodes a native C-terminal amino acid sequence of the epitope, and wherein the 3′ linker sequence encodes a peptide that is at least 3 amino acids in length, and U is each of a PADRE class II sequence and a Tetanus toxoid MHC class II sequence. The above antigen encoding sequence formula in some instances only describes the portion of an antigen cassette encoding concatenated epitope sequences, such as concatenated T cell epitopes.

In one example, elements present include where b=1, d=1, e=1, g=1, h=1, X=18, Y=2, and the vector backbone comprises a Venezuelan equine encephalitis virus vector, a=0, and the antigen cassette is operably linked to an endogenous 26S promoter, and the at least one polyadenylation poly(A) sequence is a poly(A) sequence of at least 80 consecutive A nucleotides (SEQ ID NO: 27940) provided by the backbone, and each N encodes a MHC class I epitope 7-15 amino acids in length, a MHC class II epitope, an epitope capable of stimulating a B cell response, or combinations thereof, L5 is a native 5′ linker sequence that encodes a native N-terminal amino acid sequence of the epitope, and wherein the 5′ linker sequence encodes a peptide that is at least 3 amino acids in length, L3 is a native 3′ linker sequence that encodes a native C-terminal amino acid sequence of the epitope, and wherein the 3′ linker sequence encodes a peptide that is at least 3 amino acids in length, and U is each of a PADRE class II sequence and a Tetanus toxoid MHC class II sequence.

The antigen encoding sequence can be described using the following formula to describe the ordered sequence of each element, from 5′ to 3′:

(Pa-(L5_{b}-N_{c}-L3_{d})_{X})_{Z}-(P2ₕ-(G5ₑ-U_{f})_{Y})_{W}-G3_{g}

wherein P and P2 comprise promoter nucleotide sequences, N comprises one of the SARS-CoV-2 derived nucleic acid sequences described herein (e.g., N encodes a polypeptide sequence as set forth in Table A, Table B, Table C, and/or Table 7), L5 comprises a 5′ linker sequence, L3 comprises a 3′ linker sequence, G5 comprises a nucleic acid sequences encoding an amino acid linker, G3 comprises one of the at least one nucleic acid sequences encoding an amino acid linker, U comprises an MHC class II epitope-encoding nucleic acid sequence, where for each X the corresponding Nc is a SARS-CoV-2 derived nucleic acid sequence, where for each Y the corresponding U_{f} is a (1) universal MHC class II epitope-encoding nucleic acid sequence, optionally wherein the at least one universal sequence comprises at least one of Tetanus toxoid and PADRE, or (2) a MHC class II SARS-CoV-2 derived epitope-encoding nucleic acid sequence. The composition and ordered sequence can be further defined by selecting the number of elements present, for example where a=0 or 1, where b=0 or 1, where c=1, where d=0 or 1, where e=0 or 1, where f=1, where g=0 or 1, where h=0 or 1, X=1 to 400, Y=0, 1, 2, 3, 4 or 5, Z=1 to 400, and W=0, 1, 2, 3, 4 or 5.

In one example, elements present include where a=0, b=1, d=1, e=1, g=1, h=0, X=10, Y=2, Z=1, and W=1, describing where no additional promoter is present (e.g. only the promoter nucleotide sequence provided by the vector backbone, such as an RNA alphavirus backbone, is present), 10 epitopes are present, a 5′ linker is present for each N, a 3′ linker is present for each N, 2 MHC class II epitopes are present, a linker is present linking the two MHC class II epitopes, a linker is present linking the 5′ end of the two MHC class II epitopes to the 3′ linker of the final MHC class I epitope, and a linker is present linking the 3′ end of the two MHC class II epitopes to the to the vector backbone. Examples of linking the 3′ end of the antigen cassette to the vector backbone include linking directly to the 3′ UTR elements provided by the vector backbone, such as a 3′ 19-nt CSE. Examples of linking the 5′ end of the antigen cassette to the vector backbone include linking directly to a promoter or 5′ UTR element of the vector backbone, such as a 26S promoter sequence, an alphavirus 5′ UTR, a 51-nt CSE, or a 24-nt CSE of an alphavirus vector backbone.

Other examples include: where a=1 describing where a promoter other than the promoter nucleotide sequence provided by the vector backbone is present; where a=1 and Z is greater than 1 where multiple promoters other than the promoter nucleotide sequence provided by the vector backbone are present each driving expression of 1 or more distinct MHC class I epitope encoding nucleic acid sequences; where h=1 describing where a separate promoter is present to drive expression of the MHC class II epitope-encoding nucleic acid sequences; and where g=0 describing the MHC class II epitope-encoding nucleic acid sequence, if present, is directly linked to the vector backbone.

Other examples include where each MHC class I epitope that is present can have a 5′ linker, a 3′ linker, neither, or both. In examples where more than one MHC class I epitope is present in the same antigen cassette, some MHC class I epitopes may have both a 5′ linker and a 3′ linker, while other MHC class I epitopes may have either a 5′ linker, a 3′ linker, or neither. In other examples where more than one MHC class I epitope is present in the same antigen cassette, some MHC class I epitopes may have either a 5′ linker or a 3′ linker, while other MHC class I epitopes may have either a 5′ linker, a 3′ linker, or neither.

In examples where more than one MHC class II epitope is present in the same antigen cassette, some MHC class II epitopes may have both a 5′ linker and a 3′ linker, while other MHC class II epitopes may have either a 5′ linker, a 3′ linker, or neither. In other examples where more than one MHC class II epitope is present in the same antigen cassette, some MHC class II epitopes may have either a 5′ linker or a 3′ linker, while other MHC class II epitopes may have either a 5′ linker, a 3′ linker, or neither.

Other examples include where each antigen that is present can have a 5′ linker, a 3′ linker, neither, or both. In examples where more than one antigen is present in the same antigen cassette, some antigens may have both a 5′ linker and a 3′ linker, while other antigens may have either a 5′ linker, a 3′ linker, or neither. In other examples where more than one antigen is present in the same antigen cassette, some antigens may have either a 5′ linker or a 3′ linker, while other antigens may have either a 5′ linker, a 3′ linker, or neither.

The promoter nucleotide sequences P and/or P2 can be the same as a promoter nucleotide sequence provided by the vector backbone, such as a RNA alphavirus backbone. For example, the promoter sequence provided by the vector backbone, Pn and P2, can each comprise a 26S subgenomic promoter or a CMV promoter. The promoter nucleotide sequences P and/or P2 can be different from the promoter nucleotide sequence provided by the vector backbone, as well as can be different from each other.

The 5′ linker L5 can be a native sequence or a non-natural sequence. Non-natural sequence include, but are not limited to, AAY, RR, and DPP. The 3′ linker L3 can also be a native sequence or a non-natural sequence. Additionally, L5 and L3 can both be native sequences, both be non-natural sequences, or one can be native and the other non-natural. For each X, the amino acid linkers can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. For each X, the amino acid linkers can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

The amino acid linker G5, for each Y, can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. For each Y, the amino acid linkers can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

The amino acid linker G3 can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. G3 can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

For each X, each N can encode a MHC class I epitope, a MHC class II epitope, an epitope capable of stimulating a B cell response, or a combination thereof. For each X, N can encode a combination of a MHC class I epitope, a MHC class II epitope, and an epitope capable of stimulating a B cell response. For each X, N can encode a combination of a MHC class I epitope and a MHC class II epitope. For each X, N can encode a combination of a MHC class I epitope and an epitope capable of stimulating a B cell response. For each X, N can encode a combination of a MHC class II epitope and an epitope capable of stimulating a B cell response. For each X, each N can encode a MHC class I epitope 7-15 amino acids in length. For each X, each N can also encodes a MHC class I epitope 5, 6, 7, 8, 9, 10, 11, 12, 13, 14, 15, 16, 17, 18, 19, 20, 21, 22, 23, 24, 25, 26, 27, 28, 29, or 30 amino acids in length. For each X, each N can also encodes a MHC class I epitope at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length. For each X, each N can encode a MHC class II epitope. For each X, each N can encode an epitope capable of stimulating a B cell response.

The cassette encoding the one or more antigens can be 700 nucleotides or less. The cassette encoding the one or more antigens can be 700 nucleotides or less and encode 2 distinct epitope-encoding nucleic acid sequences (e.g., encode 2 distinct SARS-CoV-2 derived nucleic acid sequence encoding an immunogenic polypeptide). The cassette encoding the one or more antigens can be 700 nucleotides or less and encode at least 2 distinct epitope-encoding nucleic acid sequences. The cassette encoding the one or more antigens can be 700 nucleotides or less and encode 3 distinct epitope-encoding nucleic acid sequences. The cassette encoding the one or more antigens can be 700 nucleotides or less and encode at least 3 distinct epitope-encoding nucleic acid sequences. The cassette encoding the one or more antigens can be 700 nucleotides or less and include 1-10, 1-5, 1, 2, 3, 4, 5, 6, 7, 8, 9, 10, or more antigens.

The cassette encoding the one or more antigens can be between 375-700 nucleotides in length. The cassette encoding the one or more antigens can be between 375-700 nucleotides in length and encode 2 distinct epitope-encoding nucleic acid sequences. The cassette encoding the one or more antigens can be between 375-700 nucleotides in length and encode at least 2 distinct epitope-encoding nucleic acid sequences. The cassette encoding the one or more antigens can be between 375-700 nucleotides in length and encode 3 distinct epitope-encoding nucleic acid sequences. The cassette encoding the one or more antigens be between 375-700 nucleotides in length and encode at least 3 distinct epitope-encoding nucleic acid sequences. The cassette encoding the one or more antigens can be between 375-700 nucleotides in length and include 1-10, 1-5, 1, 2, 3, 4, 5, 6, 7, 8, 9, 10, or more antigens.

The cassette encoding the one or more antigens can be 600, 500, 400, 300, 200, or 100 nucleotides in length or less. The cassette encoding the one or more antigens can be 600, 500, 400, 300, 200, or 100 nucleotides in length or less and encode 2 distinct epitope-encoding nucleic acid sequences. The cassette encoding the one or more antigens can be 600, 500, 400, 300, 200, or 100 nucleotides in length or less and encode at least 2 distinct epitope-encoding nucleic acid sequences. The cassette encoding the one or more antigens can be 600, 500, 400, 300, 200, or 100 nucleotides in length or less and encode 3 distinct epitope-encoding nucleic acid sequences. The cassette encoding the one or more antigens can be 600, 500, 400, 300, 200, or 100 nucleotides in length or less and encode at least 3 distinct epitope-encoding nucleic acid sequences. The cassette encoding the one or more antigens can be 600, 500, 400, 300, 200, or 100 nucleotides in length or less and include 1-10, 1-5, 1, 2, 3, 4, 5, 6, 7, 8, 9, 10, or more antigens.

The cassette encoding the one or more antigens can be between 375-600, between 375-500, or between 375-400 nucleotides in length. The cassette encoding the one or more antigens can be between 375-600, between 375-500, or between 375-400 nucleotides in length and encode 2 distinct epitope-encoding nucleic acid sequences. The cassette encoding the one or more antigens can be between 375-600, between 375-500, or between 375-400 nucleotides in length and encode at least 2 distinct epitope-encoding nucleic acid sequences. The cassette encoding the one or more antigens can be between 375-600, between 375-500, or between 375-400 nucleotides in length and encode 3 distinct epitope-encoding nucleic acid sequences. The cassette encoding the one or more antigens can be between 375-600, between 375-500, or between 375-400 nucleotides in length and encode at least 3 distinct epitope-encoding nucleic acid sequences. The cassette encoding the one or more antigens can be between 375-600, between 375-500, or between 375-400 nucleotides in length and include 1-10, 1-5, 1, 2, 3, 4, 5, 6, 7, 8, 9, 10, or more antigens.

### V.B. Additional Considerations for Vaccine Design and Manufacture

After all of the above antigen filters are applied, more candidate antigens may still be available for vaccine inclusion than the vaccine technology can support. Additionally, uncertainty about various aspects of the antigen analysis may remain and tradeoffs may exist between different properties of candidate vaccine antigens. Thus, in place of predetermined filters at each step of the selection process, an integrated multi-dimensional model can be considered that places candidate antigens in a space with at least the following axes and optimizes selection using an integrative approach.
1. Risk of auto-immunity or tolerance (risk of germline) (lower risk of auto-immunity is typically preferred)
2. Probability of sequencing artifact (lower probability of artifact is typically preferred)
3. Probability of immunogenicity (higher probability of immunogenicity is typically preferred)
4. Probability of presentation (higher probability of presentation is typically preferred)
5. Gene expression (higher expression is typically preferred)
6. Coverage of HLA genes (larger number of HLA molecules involved in the presentation of a set of antigens may lower the probability that an infected cell will escape immune attack via downregulation or mutation of HLA molecules)
7. Coverage of HLA classes (covering both HLA-I and HLA-II may increase the probability of therapeutic response and decrease the probability of infectious disease escape)

Additionally, optionally, antigens can be deprioritized (e.g., excluded) from the vaccination if they are predicted to be presented by HLA alleles lost or inactivated in either all or part of the patient's infected cell. HLA allele loss can occur by either somatic mutation, loss of heterozygosity, or homozygous deletion of the locus. Methods for detection of HLA allele somatic mutation are well known in the art, e.g. (Shukla et al., 2015). Methods for detection of somatic LOH and homozygous deletion (including for HLA locus) are likewise well described. (Carter et al., 2012; McGranahan et al., 2017; Van Loo et al., 2010). Antigens can also be deprioritized if mass-spectrometry data indicates a predicted antigen is not presented by a predicted HLA allele.

### V.C. Self-Amplifying RNA Vectors

In general, all self-amplifying RNA (SAM) vectors contain a self-amplifying backbone derived from a self-replicating virus. The term “self-amplifying backbone” refers to minimal sequence(s) of a self-replicating virus that allows for self-replication of the viral genome. For example, minimal sequences that allow for self-replication of an alphavirus can include conserved sequences for nonstructural protein-mediated amplification (e.g., a nonstructural protein 1 (nsP1) gene, a nsP2 gene, a nsP3 gene, a nsP4 gene, and/or a polyA sequence). A self-amplifying backbone can also include sequences for expression of subgenomic viral RNA (e.g., a 26S promoter element for an alphavirus). SAM vectors can be positive-sense RNA polynucleotides or negative-sense RNA polynucleotides, such as vectors with backbones derived from positive-sense or negative-sense self-replicating viruses. Self-replicating viruses include, but are not limited to, alphaviruses, flaviviruses (e.g., Kunjin virus), measles viruses, and rhabdoviruses (e.g., rabies virus and vesicular stomatitis virus). Examples of SAM vector systems derived from self-replicating viruses are described in greater detail in Lundstrom (Molecules. 2018 Dec. 13; 23(12). pii: E3310. doi: 10.3390/molecules23123310), herein incorporated by reference for all purposes.

### V.C.1. Alphavirus Biology

Alphaviruses are members of the family Togaviridae, and are positive-sense single stranded RNA viruses. Members are typically classified as either Old World, such as Sindbis, Ross River, Mayaro, Chikungunya, and Semliki Forest viruses, or New World, such as eastern equine encephalitis, Aura, Fort Morgan, or Venezuelan equine encephalitis virus and its derivative strain TC-83 (Strauss Microbrial Review 1994). A natural alphavirus genome is typically around 12 kb in length, the first two-thirds of which contain genes encoding non-structural proteins (nsPs) that form RNA replication complexes for self-replication of the viral genome, and the last third of which contains a subgenomic expression cassette encoding structural proteins for virion production (Frolov RNA 2001).

A model lifecycle of an alphavirus involves several distinct steps (Strauss Microbrial Review 1994, Jose Future Microbiol 2009). Following virus attachment to a host cell, the virion fuses with membranes within endocytic compartments resulting in the eventual release of genomic RNA into the cytosol. The genomic RNA, which is in a plus-strand orientation and comprises a 5′ methylguanylate cap and 3′ polyA tail, is translated to produce non-structural proteins nsP1-4 that form the replication complex. Early in infection, the plus-strand is then replicated by the complex into a minus-stand template. In the current model, the replication complex is further processed as infection progresses, with the resulting processed complex switching to transcription of the minus-strand into both full-length positive-strand genomic RNA, as well as the 26S subgenomic positive-strand RNA containing the structural genes. Several conserved sequence elements (CSEs) of alphavirus have been identified to potentially play a role in the various RNA replication steps including; a complement of the 5′ UTR in the replication of plus-strand RNAs from a minus-strand template, a 51-nt CSE in the replication of minus-strand synthesis from the genomic template, a 24-nt CSE in the junction region between the nsPs and the 26S RNA in the transcription of the subgenomic RNA from the minus-strand, and a 3′ 19-nt CSE in minus-strand synthesis from the plus-strand template.

Following the replication of the various RNA species, virus particles are then typically assembled in the natural lifecycle of the virus. The 26S RNA is translated and the resulting proteins further processed to produce the structural proteins including capsid protein, glycoproteins E1 and E2, and two small polypeptides E3 and 6K (Strauss 1994). Encapsidation of viral RNA occurs, with capsid proteins normally specific for only genomic RNA being packaged, followed by virion assembly and budding at the membrane surface.

### V.C.2. Alphavirus as a Delivery Vector

Alphaviruses (including alphavirus sequences, features, and other elements) can be used to generate alphavirus-based delivery vectors (also be referred to as alphavirus vectors, alphavirus viral vectors, alphavirus vaccine vectors, self-replicating RNA (srRNA) vectors, or self-amplifying RNA (samRNA) vectors). Alphaviruses have previously been engineered for use as expression vector systems (Pushko 1997, Rheme 2004). Alphaviruses offer several advantages, particularly in a vaccine setting where heterologous antigen expression can be desired. Due to its ability to self-replicate in the host cytosol, alphavirus vectors are generally able to produce high copy numbers of the expression cassette within a cell resulting in a high level of heterologous antigen production. Additionally, the vectors are generally transient, resulting in improved biosafety as well as reduced induction of immunological tolerance to the vector. The public, in general, also lacks pre-existing immunity to alphavirus vectors as compared to other standard viral vectors, such as human adenovirus. Alphavirus based vectors also generally result in cytotoxic responses to infected cells. Cytotoxicity, to a certain degree, can be important in a vaccine setting to properly illicit an immune response to the heterologous antigen expressed. However, the degree of desired cytotoxicity can be a balancing act, and thus several attenuated alphaviruses have been developed, including the TC-83 strain of VEE. Thus, an example of an antigen expression vector described herein can utilize an alphavirus backbone that allows for a high level of antigen expression, stimulates a robust immune response to antigen, does not stimulate an immune response to the vector itself, and can be used in a safe manner. Furthermore, the antigen expression cassette can be designed to stimulate different levels of an immune response through optimization of which alphavirus sequences the vector uses, including, but not limited to, sequences derived from VEE or its attenuated derivative TC-83.

Several expression vector design strategies have been engineered using alphavirus sequences (Pushko 1997). In one strategy, a alphavirus vector design includes inserting a second copy of the 26S promoter sequence elements downstream of the structural protein genes, followed by a heterologous gene (Frolov 1993). Thus, in addition to the natural non-structural and structural proteins, an additional subgenomic RNA is produced that expresses the heterologous protein. In this system, all the elements for production of infectious virions are present and, therefore, repeated rounds of infection of the expression vector in non-infected cells can occur.

Another expression vector design makes use of helper virus systems (Pushko 1997). In this strategy, the structural proteins are replaced by a heterologous gene. Thus, following self-replication of viral RNA mediated by still intact non-structural genes, the 26S subgenomic RNA provides for expression of the heterologous protein. Traditionally, additional vectors that expresses the structural proteins are then supplied in trans, such as by co-transfection of a cell line, to produce infectious virus. A system is described in detail in U.S. Pat. No. 8,093,021, which is herein incorporated by reference in its entirety, for all purposes. The helper vector system provides the benefit of limiting the possibility of forming infectious particles and, therefore, improves biosafety. In addition, the helper vector system reduces the total vector length, potentially improving the replication and expression efficiency. Thus, an example of an antigen expression vector described herein can utilize an alphavirus backbone wherein the structural proteins are replaced by an antigen cassette, the resulting vector both reducing biosafety concerns, while at the same time promoting efficient expression due to the reduction in overall expression vector size.

### V.C.3. Self-Amplifying Virus Production In Vitro

A convenient technique well-known in the art for RNA production is in vitro transcription (IVT). In this technique, a DNA template of the desired vector is first produced by techniques well-known to those in the art, including standard molecular biology techniques such as cloning, restriction digestion, ligation, gene synthesis, and polymerase chain reaction (PCR).

The DNA template contains a RNA polymerase promoter at the 5′ end of the sequence desired to be transcribed into RNA (e.g., SAM). Promoters include, but are not limited to, bacteriophage polymerase promoters such as T3, T7, K11, or SP6. Depending on the specific RNA polymerase promoter sequence chosen, additional 5′ nucleotides can transcribed in addition to the desired sequence. For example, the canonical T7 promoter can be referred to by the sequence TAATACGACTCACTATAGG (SEQ ID NO: 118), in which an IVT reaction using the DNA template TAATACGACTCACTATAGGN (SEQ ID NO: 119) for the production of desired sequence N will result in the mRNA sequence GG-N. In general, and without wishing to be bound by theory, T7 polymerase more efficiently transcribes RNA transcripts beginning with guanosine. In instances where additional 5′ nucleotides are not desired (e.g., no additional GG), the RNA polymerase promoter contained in the DNA template can be a sequence the results in transcripts containing only the 5′ nucleotides of the desired sequence, e.g., a SAM having the native 5′ sequence of the self-replicating virus from which the SAM vector is derived. For example, a minimal T7 promoter can be referred to by the sequence TAATACGACTCACTATA (SEQ ID NO: 120), in which an IVT reaction using the DNA template TAATACGACTCACTATAN (SEQ ID NO: 121) for the production of desired sequence N will result in the mRNA sequence N. Likewise, a minimal SP6 promoter referred to by the sequence ATTTAGGTGACACTATA (SEQ ID NO: 122) can be used to generate transcripts without additional 5′ nucleotides. In a typical IVT reaction, the DNA template is incubated with the appropriate RNA polymerase enzyme, buffer agents, and nucleotides (NTPs).

The resulting RNA polynucleotide can optionally be further modified including, but limited to, addition of a 5′ cap structure such as 7-methylguanosine or a related structure, and optionally modifying the 3′ end to include a polyadenylate (polyA) tail. In a modified IVT reaction, RNA is capped with a 5′ cap structure co-transcriptionally through the addition of cap analogues during IVT. Cap analogues can include dinucleotide (m⁷G-ppp-N) cap analogues or trinucleotide (m⁷G-ppp-N-N) cap analogues, where N represents a nucleotide or modified nucleotide (e.g., ribonucleosides including, but not limited to, adenosine, guanosine, cytidine, and uradine). Exemplary cap analogues and their use in IVT reactions are also described in greater detail in U.S. Pat. No. 10,519,189, herein incorporated by reference for all purposes. As discussed, T7 polymerase more efficiently transcribes RNA transcripts beginning with guanosine. To improve transcription efficiency in templates that do not begin with guanosine, a trinucleotide cap analogue (m⁷G-ppp-N-N) can be used. The trinucleotide cap analogue can increase transcription efficiency 2, 3, 4, 5, 6, 7, 8, 9, 10, 11, 12, 13, 14, 15, 16, 17, 18, 19, 20-fold or more relative to an IVT reaction using a dinucleotide cap analogue (m⁷G-ppp-N).

A 5′ cap structure can also be added following transcription, such as using a vaccinia capping system (e.g., NEB Cat. No. M2080) containing mRNA 2′-O-methyltransferase and S-Adenosyl methionine.

The resulting RNA polynucleotide can optionally be further modified separately from or in addition to the capping techniques described including, but limited to, modifying the 3′ end to include a polyadenylate (polyA) tail.

The RNA can then be purified using techniques well-known in the field, such as phenol-chloroform extraction.

### V.C.4. Delivery Via Lipid Nanoparticle

An important aspect to consider in vaccine vector design is immunity against the vector itself (Riley 2017). This may be in the form of preexisting immunity to the vector itself, such as with certain human adenovirus systems, or in the form of developing immunity to the vector following administration of the vaccine. The latter is an important consideration if multiple administrations of the same vaccine are performed, such as separate priming and boosting doses, or if the same vaccine vector system is to be used to deliver different antigen cassettes.

In the case of alphavirus vectors, the standard delivery method is the previously discussed helper virus system that provides capsid, E1, and E2 proteins in trans to produce infectious viral particles. However, it is important to note that the E1 and E2 proteins are often major targets of neutralizing antibodies (Strauss 1994). Thus, the efficacy of using alphavirus vectors to deliver antigens of interest to target cells may be reduced if infectious particles are targeted by neutralizing antibodies.

An alternative to viral particle mediated gene delivery is the use of nanomaterials to deliver expression vectors (Riley 2017). Nanomaterial vehicles, importantly, can be made of non-immunogenic materials and generally avoid stimulating immunity to the delivery vector itself. These materials can include, but are not limited to, lipids, inorganic nanomaterials, and other polymeric materials. Lipids can be cationic, anionic, or neutral. The materials can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins.

Lipid nanoparticles (LNPs) are an attractive delivery system due to the amphiphilic nature of lipids enabling formation of membranes and vesicle like structures (Riley 2017). In general, these vesicles deliver the expression vector by absorbing into the membrane of target cells and releasing nucleic acid into the cytosol. In addition, LNPs can be further modified or functionalized to facilitate targeting of specific cell types. Another consideration in LNP design is the balance between targeting efficiency and cytotoxicity. Lipid compositions generally include defined mixtures of cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids.

Nucleic-acid vectors, such as expression vectors, exposed directly to serum can have several undesirable consequences, including degradation of the nucleic acid by serum nucleases or off-target stimulation of the immune system by the free nucleic acids. Therefore, encapsulation of the alphavirus vector can be used to avoid degradation, while also avoiding potential off-target effects. In certain examples, an alphavirus vector is fully encapsulated within the delivery vehicle, such as within the aqueous interior of an LNP. Encapsulation of the alphavirus vector within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with the alphavirus delivery vector and other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the expression vectors can be further treated or modified to prepare them for administration.

### V.D. Chimpanzee Adenovirus (ChAd)

### V.D.1. Viral Delivery with Chimpanzee Adenovirus

Vaccine compositions for delivery of one or more antigens (e.g., via an antigen cassette) can be created by providing adenovirus nucleotide sequences of chimpanzee origin, a variety of novel vectors, and cell lines expressing chimpanzee adenovirus genes. A nucleotide sequence of a chimpanzee C68 adenovirus (also referred to herein as ChAdV68) can be used in a vaccine composition for antigen delivery (See SEQ ID NO: 1). Use of C68 adenovirus derived vectors is described in further detail in U.S. Pat. No. 6,083,716, which is herein incorporated by reference in its entirety, for all purposes.

In a further aspect, provided herein is a recombinant adenovirus comprising the DNA sequence of a chimpanzee adenovirus such as C68 and an antigen cassette operatively linked to regulatory sequences directing its expression. The recombinant virus is capable of infecting a mammalian, preferably a human, cell and capable of expressing the antigen cassette product in the cell. In this vector, the native chimpanzee E1 gene, and/or E3 gene, and/or E4 gene can be deleted. An antigen cassette can be inserted into any of these sites of gene deletion. The antigen cassette can include an antigen against which a primed immune response is desired.

In another aspect, provided herein is a mammalian cell infected with a chimpanzee adenovirus such as C68.

In still a further aspect, a novel mammalian cell line is provided which expresses a chimpanzee adenovirus gene (e.g., from C68) or functional fragment thereof.

In still a further aspect, provided herein is a method for delivering an antigen cassette into a mammalian cell comprising the step of introducing into the cell an effective amount of a chimpanzee adenovirus, such as C68, that has been engineered to express the antigen cassette.

Still another aspect provides a method for stimulating an immune response in a mammalian host. The method can comprise the step of administering to the host an effective amount of a recombinant chimpanzee adenovirus, such as C68, comprising an antigen cassette that encodes one or more antigens from the infection against which the immune response is targeted.

Still another aspect provides a method for stimulating an immune response in a mammalian host to treat or prevent a disease in a subject, such as an infectious disease. The method can comprise the step of administering to the host an effective amount of a recombinant chimpanzee adenovirus, such as C68, comprising an antigen cassette that encodes one or more antigens, such as from the infectious disease against which the immune response is targeted.

Also disclosed is a non-simian mammalian cell that expresses a chimpanzee adenovirus gene obtained from the sequence of SEQ ID NO: 1. The gene can be selected from the group consisting of the adenovirus E1A, E1B, E2A, E2B, E3, E4, L1, L2, L3, L4 and L5 of SEQ ID NO: 1.

Also disclosed is a nucleic acid molecule comprising a chimpanzee adenovirus DNA sequence comprising a gene obtained from the sequence of SEQ ID NO: 1. The gene can be selected from the group consisting of said chimpanzee adenovirus E1A, E1B, E2A, E2B, E3, E4, L1, L2, L3, L4 and L5 genes of SEQ ID NO: 1. In some aspects the nucleic acid molecule comprises SEQ ID NO: 1. In some aspects the nucleic acid molecule comprises the sequence of SEQ ID NO: 1, lacking at least one gene selected from the group consisting of E1A, E1B, E2A, E2B, E3, E4, L1, L2, L3, L4 and L5 genes of SEQ ID NO: 1.

Also disclosed is a vector comprising a chimpanzee adenovirus DNA sequence obtained from SEQ ID NO: 1 and an antigen cassette operatively linked to one or more regulatory sequences which direct expression of the cassette in a heterologous host cell, optionally wherein the chimpanzee adenovirus DNA sequence comprises at least the cis-elements necessary for replication and virion encapsidation, the cis-elements flanking the antigen cassette and regulatory sequences. In some aspects, the chimpanzee adenovirus DNA sequence comprises a gene selected from the group consisting of E1A, E1B, E2A, E2B, E3, E4, L1, L2, L3, L4 and L5 gene sequences of SEQ ID NO: 1. In some aspects the vector can lack the E1A and/or E1B gene.

Also disclosed herein is a adenovirus vector comprising: a partially deleted E4 gene comprising a deleted or partially-deleted E4orf2 region and a deleted or partially-deleted E4orf3 region, and optionally a deleted or partially-deleted E4orf4 region. The partially deleted E4 can comprise an E4 deletion of at least nucleotides 34,916 to 35,642 of the sequence shown in SEQ ID NO:1, and wherein the vector comprises at least nucleotides 2 to 36,518 of the sequence set forth in SEQ ID NO:1. The partially deleted E4 can comprise an E4 deletion of at least a partial deletion of nucleotides 34,916 to 34,942 of the sequence shown in SEQ ID NO:1, at least a partial deletion of nucleotides 34,952 to 35,305 of the sequence shown in SEQ ID NO:1, and at least a partial deletion of nucleotides 35,302 to 35,642 of the sequence shown in SEQ ID NO:1, and wherein the vector comprises at least nucleotides 2 to 36,518 of the sequence set forth in SEQ ID NO:1 The partially deleted E4 can comprise an E4 deletion of at least nucleotides 34,980 to 36,516 of the sequence shown in SEQ ID NO:1, and wherein the vector comprises at least nucleotides 2 to 36,518 of the sequence set forth in SEQ ID NO:1. The partially deleted E4 can comprise an E4 deletion of at least nucleotides 34,979 to 35,642 of the sequence shown in SEQ ID NO:1, and wherein the vector comprises at least nucleotides 2 to 36,518 of the sequence set forth in SEQ ID NO:1. The partially deleted E4 can comprise an E4 deletion of at least a partial deletion of E4Orf2, a fully deleted E4Orf3, and at least a partial deletion of E4Orf4. The partially deleted E4 can comprise an E4 deletion of at least a partial deletion of E4Orf2, at least a partial deletion of E4Orf3, and at least a partial deletion of E4Orf4. The partially deleted E4 can comprise an E4 deletion of at least a partial deletion of E4Orf1, a fully deleted E4Orf2, and at least a partial deletion of E4Orf3. The partially deleted E4 can comprise an E4 deletion of at least a partial deletion of E4Orf2 and at least a partial deletion of E4Orf3. The partially deleted E4 can comprise an E4 deletion between the start site of E4Orf1 to the start site of E4Orf5. The partially deleted E4 can be an E4 deletion adjacent to the start site of E4Orf1. The partially deleted E4 can be an E4 deletion adjacent to the start site of E4Orf2. The partially deleted E4 can be an E4 deletion adjacent to the start site of E4Orf3. The partially deleted E4 can be an E4 deletion adjacent to the start site of E4Orf4. The E4 deletion can be at least 50, at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900, or at least 2000 nucleotides. The E4 deletion can be at least 700 nucleotides. The E4 deletion can be at least 1500 nucleotides. The E4 deletion can be 50 or less, 100 or less, 200 or less, 300 or less, 400 or less, 500 or less, 600 or less, 700 or less, 800 or less, 900 or less, 1000 or less, 1100 or less, 1200 or less, 1300 or less, 1400 or less, 1500 or less, 1600 or less, 1700 or less, 1800 or less, 1900 or less, or 2000 or less nucleotides. The E4 deletion can be 750 nucleotides or less. The E4 deletion can be at least 1550 nucleotides or less.

Also disclosed herein is a host cell transfected with a vector disclosed herein such as a C68 vector engineered to expression an antigen cassette. Also disclosed herein is a human cell that expresses a selected gene introduced therein through introduction of a vector disclosed herein into the cell.

Also disclosed herein is a method for delivering an antigen cassette to a mammalian cell comprising introducing into said cell an effective amount of a vector disclosed herein such as a C68 vector engineered to expression the antigen cassette.

Also disclosed herein is a method for producing an antigen comprising introducing a vector disclosed herein into a mammalian cell, culturing the cell under suitable conditions and producing the antigen.

### V.D.2. E1-Expressing Complementation Cell Lines

To generate recombinant chimpanzee adenoviruses (Ad) deleted in any of the genes described herein, the function of the deleted gene region, if essential to the replication and infectivity of the virus, can be supplied to the recombinant virus by a helper virus or cell line, i.e., a complementation or packaging cell line. For example, to generate a replication-defective chimpanzee adenovirus vector, a cell line can be used which expresses the E1 gene products of the human or chimpanzee adenovirus; such a cell line can include HEK293 or variants thereof. The protocol for the generation of the cell lines expressing the chimpanzee E1 gene products (Examples 3 and 4 of U.S. Pat. No. 6,083,716) can be followed to generate a cell line which expresses any selected chimpanzee adenovirus gene.

An AAV augmentation assay can be used to identify a chimpanzee adenovirus E1-expressing cell line. This assay is useful to identify E1 function in cell lines made by using the E1 genes of other uncharacterized adenoviruses, e.g., from other species. That assay is described in Example 4B of U.S. Pat. No. 6,083,716.

A selected chimpanzee adenovirus gene, e.g., E1, can be under the transcriptional control of a promoter for expression in a selected parent cell line. Inducible or constitutive promoters can be employed for this purpose. Among inducible promoters are included the sheep metallothionine promoter, inducible by zinc, or the mouse mammary tumor virus (MMTV) promoter, inducible by a glucocorticoid, particularly, dexamethasone. Other inducible promoters, such as those identified in International patent application WO95/13392, incorporated by reference herein can also be used in the production of packaging cell lines. Constitutive promoters in control of the expression of the chimpanzee adenovirus gene can be employed also.

A parent cell can be selected for the generation of a novel cell line expressing any desired C68 gene. Without limitation, such a parent cell line can be HeLa [ATCC Accession No. CCL 2], A549 [ATCC Accession No. CCL 185], KB [CCL 17], Detroit [e.g., Detroit 510, CCL 72] and WI-38 [CCL 75] cells. Other suitable parent cell lines can be obtained from other sources. Parent cell lines can include CHO, HEK293 or variants thereof, 911, HeLa, A549, LP-293, PER.C6, or AE1-2a.

An E1-expressing cell line can be useful in the generation of recombinant chimpanzee adenovirus E1 deleted vectors. Cell lines constructed using essentially the same procedures that express one or more other chimpanzee adenoviral gene products are useful in the generation of recombinant chimpanzee adenovirus vectors deleted in the genes that encode those products. Further, cell lines which express other human Ad E1 gene products are also useful in generating chimpanzee recombinant Ads.

### V.D.3. Recombinant Viral Particles as Vectors

The compositions disclosed herein can comprise viral vectors, that deliver at least one antigen to cells. Such vectors comprise a chimpanzee adenovirus DNA sequence such as C68 and an antigen cassette operatively linked to regulatory sequences which direct expression of the cassette. The C68 vector is capable of expressing the cassette in an infected mammalian cell. The C68 vector can be functionally deleted in one or more viral genes. An antigen cassette comprises at least one antigen under the control of one or more regulatory sequences such as a promoter. Optional helper viruses and/or packaging cell lines can supply to the chimpanzee viral vector any necessary products of deleted adenoviral genes.

The term “functionally deleted” means that a sufficient amount of the gene region is removed or otherwise altered, e.g., by mutation or modification, so that the gene region is no longer capable of producing one or more functional products of gene expression. Mutations or modifications that can result in functional deletions include, but are not limited to, nonsense mutations such as introduction of premature stop codons and removal of canonical and non-canonical start codons, mutations that alter mRNA splicing or other transcriptional processing, or combinations thereof. If desired, the entire gene region can be removed.

Modifications of the nucleic acid sequences forming the vectors disclosed herein, including sequence deletions, insertions, and other mutations may be generated using standard molecular biological techniques and are within the scope of this invention.

### V.D.4. Construction of The Viral Plasmid Vector

The chimpanzee adenovirus C68 vectors useful in this invention include recombinant, defective adenoviruses, that is, chimpanzee adenovirus sequences functionally deleted in the E1a or E1b genes, and optionally bearing other mutations, e.g., temperature-sensitive mutations or deletions in other genes. It is anticipated that these chimpanzee sequences are also useful in forming hybrid vectors from other adenovirus and/or adeno-associated virus sequences. Homologous adenovirus vectors prepared from human adenoviruses are described in the published literature [see, for example, Kozarsky I and II, cited above, and references cited therein, U.S. Pat. No. 5,240,846].

In the construction of useful chimpanzee adenovirus C68 vectors for delivery of an antigen cassette to a human (or other mammalian) cell, a range of adenovirus nucleic acid sequences can be employed in the vectors. A vector comprising minimal chimpanzee C68 adenovirus sequences can be used in conjunction with a helper virus to produce an infectious recombinant virus particle. The helper virus provides essential gene products required for viral infectivity and propagation of the minimal chimpanzee adenoviral vector. When only one or more selected deletions of chimpanzee adenovirus genes are made in an otherwise functional viral vector, the deleted gene products can be supplied in the viral vector production process by propagating the virus in a selected packaging cell line that provides the deleted gene functions in trans.

### V.D.5. Recombinant Minimal Adenovirus

A minimal chimpanzee Ad C68 virus is a viral particle containing just the adenovirus cis-elements necessary for replication and virion encapsidation. That is, the vector contains the cis-acting 5′ and 3′ inverted terminal repeat (ITR) sequences of the adenoviruses (which function as origins of replication) and the native 5′ packaging/enhancer domains (that contain sequences necessary for packaging linear Ad genomes and enhancer elements for the E1 promoter). See, for example, the techniques described for preparation of a “minimal” human Ad vector in International Patent Application WO96/13597 and incorporated herein by reference.

### V.D.6. Other Defective Adenoviruses

Recombinant, replication-deficient adenoviruses can also contain more than the minimal chimpanzee adenovirus sequences. These other Ad vectors can be characterized by deletions of various portions of gene regions of the virus, and infectious virus particles formed by the optional use of helper viruses and/or packaging cell lines.

As one example, suitable vectors may be formed by deleting all or a sufficient portion of the C68 adenoviral immediate early gene E1a and delayed early gene E1b, so as to eliminate their normal biological functions. Replication-defective E1-deleted viruses are capable of replicating and producing infectious virus when grown on a chimpanzee adenovirus-transformed, complementation cell line containing functional adenovirus E1a and E1b genes which provide the corresponding gene products in trans. Based on the homologies to known adenovirus sequences, it is anticipated that, as is true for the human recombinant E1-deleted adenoviruses of the art, the resulting recombinant chimpanzee adenovirus is capable of infecting many cell types and can express antigen(s), but cannot replicate in most cells that do not carry the chimpanzee E1 region DNA unless the cell is infected at a very high multiplicity of infection.

As another example, all or a portion of the C68 adenovirus delayed early gene E3 can be eliminated from the chimpanzee adenovirus sequence which forms a part of the recombinant virus.

Chimpanzee adenovirus C68 vectors can also be constructed having a deletion of the E4 gene. Still another vector can contain a deletion in the delayed early gene E2a.

Deletions can also be made in any of the late genes L1 through L5 of the chimpanzee C68 adenovirus genome. Similarly, deletions in the intermediate genes IX and IVa2 can be useful for some purposes. Other deletions may be made in the other structural or non-structural adenovirus genes.

The above discussed deletions can be used individually, i.e., an adenovirus sequence can contain deletions of E1 only. Alternatively, deletions of entire genes or portions thereof effective to destroy or reduce their biological activity can be used in any combination. For example, in one exemplary vector, the adenovirus C68 sequence can have deletions of the E1 genes and the E4 gene, or of the E1, E2a and E3 genes, or of the E1 and E3 genes, or of E1, E2a and E4 genes, with or without deletion of E3, and so on. As discussed above, such deletions can be used in combination with other mutations, such as temperature-sensitive mutations, to achieve a desired result.

The cassette comprising antigen(s) be inserted optionally into any deleted region of the chimpanzee C68 Ad virus. Alternatively, the cassette can be inserted into an existing gene region to disrupt the function of that region, if desired.

### V.D.7. Helper Viruses

Depending upon the chimpanzee adenovirus gene content of the viral vectors employed to carry the antigen cassette, a helper adenovirus or non-replicating virus fragment can be used to provide sufficient chimpanzee adenovirus gene sequences to produce an infective recombinant viral particle containing the cassette.

Useful helper viruses contain selected adenovirus gene sequences not present in the adenovirus vector construct and/or not expressed by the packaging cell line in which the vector is transfected. A helper virus can be replication-defective and contain a variety of adenovirus genes in addition to the sequences described above. The helper virus can be used in combination with the E1-expressing cell lines described herein.

For C68, the “helper” virus can be a fragment formed by clipping the C terminal end of the C68 genome with SspI, which removes about 1300 bp from the left end of the virus. This clipped virus is then co-transfected into an E1-expressing cell line with the plasmid DNA, thereby forming the recombinant virus by homologous recombination with the C68 sequences in the plasmid.

Helper viruses can also be formed into poly-cation conjugates as described in Wu et al, J. Biol. Chem., 264:16985-16987 (1989); K. J. Fisher and J. M. Wilson, Biochem. J., 299:49 (Apr. 1, 1994). Helper virus can optionally contain a reporter gene. A number of such reporter genes are known to the art. The presence of a reporter gene on the helper virus which is different from the antigen cassette on the adenovirus vector allows both the Ad vector and the helper virus to be independently monitored. This second reporter is used to enable separation between the resulting recombinant virus and the helper virus upon purification.

### V.D.8. Assembly of Viral Particle and Infection of a Cell Line

Assembly of the selected DNA sequences of the adenovirus, the antigen cassette, and other vector elements into various intermediate plasmids and shuttle vectors, and the use of the plasmids and vectors to produce a recombinant viral particle can all be achieved using conventional techniques. Such techniques include conventional cloning techniques of cDNA, in vitro recombination techniques (e.g., Gibson assembly), use of overlapping oligonucleotide sequences of the adenovirus genomes, polymerase chain reaction, and any suitable method which provides the desired nucleotide sequence. Standard transfection and co-transfection techniques are employed, e.g., CaPO4 precipitation techniques or liposome-mediated transfection methods such as lipofectamine. Other conventional methods employed include homologous recombination of the viral genomes, plaquing of viruses in agar overlay, methods of measuring signal generation, and the like.

For example, following the construction and assembly of the desired antigen cassette-containing viral vector, the vector can be transfected in vitro in the presence of a helper virus into the packaging cell line. Homologous recombination occurs between the helper and the vector sequences, which permits the adenovirus-antigen sequences in the vector to be replicated and packaged into virion capsids, resulting in the recombinant viral vector particles.

The resulting recombinant chimpanzee C68 adenoviruses are useful in transferring an antigen cassette to a selected cell. In in vivo experiments with the recombinant virus grown in the packaging cell lines, the E1-deleted recombinant chimpanzee adenovirus demonstrates utility in transferring a cassette to a non-chimpanzee, preferably a human, cell.

### V.D.9. Use of the Recombinant Virus Vectors

The resulting recombinant chimpanzee C68 adenovirus containing the antigen cassette (produced by cooperation of the adenovirus vector and helper virus or adenoviral vector and packaging cell line, as described above) thus provides an efficient gene transfer vehicle which can deliver antigen(s) to a subject in vivo or ex vivo.

The above-described recombinant vectors are administered to humans according to published methods for gene therapy. A chimpanzee viral vector bearing an antigen cassette can be administered to a patient, preferably suspended in a biologically compatible solution or pharmaceutically acceptable delivery vehicle. A suitable vehicle includes sterile saline. Other aqueous and non-aqueous isotonic sterile injection solutions and aqueous and non-aqueous sterile suspensions known to be pharmaceutically acceptable carriers and well known to those of skill in the art may be employed for this purpose.

The chimpanzee adenoviral vectors are administered in sufficient amounts to transduce the human cells and to provide sufficient levels of antigen transfer and expression to provide a therapeutic benefit without undue adverse or with medically acceptable physiological effects, which can be determined by those skilled in the medical arts. Conventional and pharmaceutically acceptable routes of administration include, but are not limited to, direct delivery to the liver, intranasal, intravenous, intramuscular, subcutaneous, intradermal, oral and other parental routes of administration. Routes of administration may be combined, if desired.

Dosages of the viral vector will depend primarily on factors such as the condition being treated, the age, weight and health of the patient, and may thus vary among patients. The dosage will be adjusted to balance the therapeutic benefit against any side effects and such dosages may vary depending upon the therapeutic application for which the recombinant vector is employed. The levels of expression of antigen(s) can be monitored to determine the frequency of dosage administration.

Recombinant, replication defective adenoviruses can be administered in a “pharmaceutically effective amount”, that is, an amount of recombinant adenovirus that is effective in a route of administration to transfect the desired cells and provide sufficient levels of expression of the selected gene to provide a vaccinal benefit, i.e., some measurable level of protective immunity. C68 vectors comprising an antigen cassette can be co-administered with adjuvant. Adjuvant can be separate from the vector (e.g., alum) or encoded within the vector, in particular if the adjuvant is a protein. Adjuvants are well known in the art.

Conventional and pharmaceutically acceptable routes of administration include, but are not limited to, intranasal, intramuscular, intratracheal, subcutaneous, intradermal, rectal, oral and other parental routes of administration. Routes of administration may be combined, if desired, or adjusted depending upon the immunogen or the disease. For example, in prophylaxis of rabies, the subcutaneous, intratracheal and intranasal routes are preferred. The route of administration primarily will depend on the nature of the disease being treated.

The levels of immunity to antigen(s) can be monitored to determine the need, if any, for boosters. Following an assessment of antibody titers in the serum, for example, optional booster immunizations may be desired

### VI. Therapeutic and Manufacturing Methods

Also provided is a method of inducing an infectious disease organism-specific (e.g. a SARS-CoV-2 specific) immune response in a subject, vaccinating against an infectious disease organism, treating and/or alleviating a symptom of an infection associated with an infectious disease organism in a subject by administering to the subject one or more antigens such as a plurality of antigens identified using methods disclosed herein.

In some aspects, a subject has been diagnosed with an infection or is at risk of an infection (e.g. Covid-19 due to a SARS-CoV-2 infection), such as age, geographical/travel, and/or work-related increased risk of or predisposition to an infection, or at risk to a seasonal and/or novel disease infection.

In some aspects, a subject is immunocompromised, such as diagnosed with and/or suspected of having cancer. A subject can include those treated with a therapy resulting in immunosuppression. For example, a subject can include those diagnosed with a hematopoietic malignancy and treated with a hematopoietic cell targeting therapy, such as a B cell malignancy treated with an anti-CD20 therapy (e.g., rituximab). In another example, a subject can include those diagnosed with multiple sclerosis [e.g., Relapsing-remitting multiple sclerosis (RRMS), Secondary-progressive multiple sclerosis (SPMS), or Primary-progressive multiple sclerosis (PPMS)] and treated with an anti-CD20 therapy.

An antigen can be administered in an amount sufficient to stimulate a CTL response. An antigen can be administered in an amount sufficient to stimulate a T cell response. An antigen can be administered in an amount sufficient to stimulate a B cell response.

An antigen can be administered alone or in combination with other therapeutic agents. Therapeutic agents can include those that target an infectious disease organism, such as an anti-viral or antibiotic agent.

The optimum amount of each antigen to be included in a vaccine composition and the optimum dosing regimen can be determined. For example, an antigen or its variant can be prepared for intravenous (i.v.) injection, sub-cutaneous (s.c.) injection, intradermal (i.d.) injection, intraperitoneal (i.p.) injection, intramuscular (i.m.) injection. Methods of injection include s.c., i.d., i.p., i.m., and i.v. Methods of DNA or RNA injection include i.d., i.m., s.c., i.p. and i.v. Other methods of administration of the vaccine composition are known to those skilled in the art.

A vaccine can be compiled so that the selection, number and/or amount of antigens present in the composition is/are tissue, infectious disease, and/or patient-specific. For instance, the exact selection of peptides can be guided by expression patterns of the parent proteins in a given tissue or guided by mutation or disease status of a patient. The selection can be dependent on the specific infectious disease (e.g. the specific SARS-CoV-2 isolate the subject is infected with or at risk for infection by), the status of the disease, the goal of the vaccination (e.g., preventative or targeting an ongoing disease), earlier treatment regimens, the immune status of the patient, and, of course, the HLA-haplotype of the patient. Furthermore, a vaccine can contain individualized components, according to personal needs of the particular patient. Examples include varying the selection of antigens according to the expression of the antigen in the particular patient or adjustments for secondary treatments following a first round or scheme of treatment.

A patient can be identified for administration of an antigen vaccine through the use of various diagnostic methods, e.g., patient selection methods described further below. Patient selection can involve identifying mutations in, or expression patterns of, one or more genes. Patient selection can involve identifying the infectious disease of an ongoing infection (e.g. the presence of a SARS-CoV-2 infection and/or the specific SARS-CoV-2 isolate). Patient selection can involve identifying risk of an infection by an infectious disease. In some cases, patient selection involves identifying the haplotype of the patient. The various patient selection methods can be performed in parallel, e.g., a sequencing diagnostic can identify both the mutations and the haplotype of a patient. The various patient selection methods can be performed sequentially, e.g., one diagnostic test identifies the mutations and separate diagnostic test identifies the haplotype of a patient, and where each test can be the same (e.g., both high-throughput sequencing) or different (e.g., one high-throughput sequencing and the other Sanger sequencing) diagnostic methods.

For a composition to be used as a vaccine for an infectious disease, antigens with similar normal self-peptides that are expressed in high amounts in normal tissues can be avoided or be present in low amounts in a composition described herein. On the other hand, if it is known that the infected cell of a patient expresses high amounts of a certain antigen, the respective pharmaceutical composition for treatment of this infection can be present in high amounts and/or more than one antigen specific for this particularly antigen or pathway of this antigen can be included.

Compositions comprising an antigen can be administered to an individual already suffering from an infection. In therapeutic applications, compositions are administered to a patient in an amount sufficient to stimulate an effective CTL response to the infectious disease organism antigen and to cure or at least partially arrest symptoms and/or complications. An amount adequate to accomplish this is defined as “therapeutically effective dose.” Amounts effective for this use will depend on, e.g., the composition, the manner of administration, the stage and severity of the disease being treated, the weight and general state of health of the patient, and the judgment of the prescribing physician. It should be kept in mind that compositions can generally be employed in serious disease states, that is, life-threatening or potentially life threatening situations, especially when the infectious disease organism has induced organ damage and/or other immune pathology. In such cases, in view of the minimization of extraneous substances and the relative nontoxic nature of an antigen, it is possible and can be felt desirable by the treating physician to administer substantial excesses of these compositions.

For therapeutic use, administration can begin at the detection or treatment of an infection. This can be followed by boosting doses until at least symptoms are substantially abated and for a period thereafter.

The pharmaceutical compositions (e.g., vaccine compositions) for therapeutic treatment are intended for parenteral, topical, nasal, oral or local administration. A pharmaceutical compositions can be administered parenterally, e.g., intravenously, subcutaneously, intradermally, or intramuscularly. The compositions can be administered to target specific infected tissues and/or cells of a subject. Disclosed herein are compositions for parenteral administration which comprise a solution of the antigen and vaccine compositions are dissolved or suspended in an acceptable carrier, e.g., an aqueous carrier. A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Antigens can also be administered via liposomes, which target them to a particular cells tissue, such as lymphoid tissue. Liposomes are also useful in increasing half-life. Liposomes include emulsions, foams, micelles, insoluble monolayers, liquid crystals, phospholipid dispersions, lamellar layers and the like. In these preparations the antigen to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired antigen can be directed to the site of lymphoid cells, where the liposomes then deliver the selected therapeutic/immunogenic compositions. Liposomes can be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension can be administered intravenously, locally, topically, etc. in a dose which varies according to, inter alia, the manner of administration, the peptide being delivered, and the stage of the disease being treated.

For therapeutic or immunization purposes, nucleic acids encoding a peptide and optionally one or more of the peptides described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Approaches for delivering nucleic acid sequences can include viral vectors, mRNA vectors, and DNA vectors with or without electroporation.

The nucleic acids can also be delivered complexed to cationic compounds, such as cationic lipids. Lipid-mediated gene delivery methods are described, for instance, in 9618372WOAWO 96/18372; 9324640WOAWO 93/24640; Mannino & Gould-Fogerite, BioTechniques 6(7): 682-691 (1988); U.S. Pat. No. 5,279,833 Rose U.S. Pat. No. 5,279,833; 9106309WOAWO 91/06309; and Felgner et al., Proc. Natl. Acad. Sci. USA 84: 7413-7414 (1987).

Antigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol.(1998) 72 (12): 9873-9880). Dependent on the packaging capacity of the above mentioned viral vector-based vaccine platforms, this approach can deliver one or more nucleotide sequences that encode one or more antigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291):1337-41, Lu et al., Efficient identification of mutated cancer antigens recognized by T cells associated with durable tumor regressions, Clin Cancer Res. (2014) 20(13):3401-10). Upon introduction into a host, infected cells express the antigens, and thereby stimulate a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of antigens, e.g., Salmonella typhi vectors, and the like will be apparent to those skilled in the art from the description herein.

A means of administering nucleic acids uses minigene constructs encoding one or multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MHC presentation of CTL epitopes can be improved by including synthetic (e.g. poly-alanine) or naturally-occurring flanking sequences adjacent to the CTL epitopes. The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Purified plasmid DNA can be prepared for injection using a variety of formulations. The simplest of these is reconstitution of lyophilized DNA in sterile phosphate-buffer saline (PBS). A variety of methods have been described, and new techniques can become available. As noted above, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Also disclosed is a method of manufacturing a vaccine, comprising performing the steps of a method disclosed herein; and producing a vaccine comprising a plurality of antigens or a subset of the plurality of antigens.

Antigens disclosed herein can be manufactured using methods known in the art. For example, a method of producing an antigen or a vector (e.g., a vector including at least one sequence encoding one or more antigens) disclosed herein can include culturing a host cell under conditions suitable for expressing the antigen or vector wherein the host cell comprises at least one polynucleotide encoding the antigen or vector, and purifying the antigen or vector. Standard purification methods include chromatographic techniques, electrophoretic, immunological, precipitation, dialysis, filtration, concentration, and chromatofocusing techniques.

Host cells can include a Chinese Hamster Ovary (CHO) cell, NSO cell, yeast, or a HEK293 cell. Host cells can be transformed with one or more polynucleotides comprising at least one nucleic acid sequence that encodes an antigen or vector disclosed herein, optionally wherein the isolated polynucleotide further comprises a promoter sequence operably linked to the at least one nucleic acid sequence that encodes the antigen or vector. In certain embodiments the isolated polynucleotide can be cDNA.

### VII. Antigen Use and Administration

A vaccination protocol can be used to dose a subject with one or more antigens and/or epitopes. A priming vaccine and a boosting vaccine can be used to dose the subject.

The priming vaccine can be based on C68 (e.g., the sequences shown in SEQ ID NO:1 or 2) or srRNA (e.g., the sequences shown in SEQ ID NO:3 or 4) and the boosting vaccine can be based on C68 (e.g., the sequences shown in SEQ ID NO:1 or 2) or srRNA/samRNA (e.g., the sequences shown in SEQ ID NO:3 or 4). Each vector typically includes a cassette that includes antigens and/or epitopes. Cassettes can include about 20 epitopes (or antigens from which the epitopes are derived), separated by spacers such as the natural sequence that normally surrounds each epitope or other non-natural spacer sequences such as AAY. Cassettes can also include MHCII antigens/epitopes such a tetanus toxoid antigen and PADRE antigen, which can be considered universal class II antigens. Cassettes can also include a targeting sequence such as a ubiquitin targeting sequence.

A priming vaccine can be injected (e.g., intramuscularly) in a subject. Bilateral injections per dose can be used. For example, one or more injections of ChAdV68 (C68) can be used (e.g., total dose 1×10¹² viral particles); one or more injections of self-amplifying RNA (samRNA or SAM), such as a dose of 3 μg, 10 μg, 30 μg, 100 μg, or 300 μg RNA can be used. A SAM priming dose of 30 μg or less can be used. A SAM priming dose of 10 μg or less can be used. A SAM priming dose of 3 μg or less can be used. One or more injections of samRNA at a dose of 30 μg or less can be used. A dose of 30 μg or less can represent the total content of RNA/samRNA administered. A dose of 30 μg or less can represent the total content of RNA/samRNA administered and include only a single distinct samRNA construct. A SAM prime of between 10-30 μg, 10-100 μg, 10-300 μg, 30-100 μg, 30-300 μg, or 100-300 μg RNA can be administered. A SAM prime of between 10-500 μg, 10-1000 μg, 30-500 μg, 30-1000 μg, or 500-1000 μg RNA can be administered. A SAM prime of at least 400 μg, at least 500 μg, at least 600 μg, at least 700 μg, at least 800 μg, at least 900 μg, at least 1000 μg RNA can be administered. A SAM prime of 10 μg, 30 μg, 100 μg, or 300 μg RNA can be administered. A SAM prime of 300 μg RNA can be administered. A SAM prime of 100 μg RNA can be administered. A SAM prime of 30 μg RNA can be administered. A SAM prime of 10 μg RNA can be administered. A SAM prime of 3 μg RNA can be administered. A SAM prime of at least 300 μg RNA can be administered. A SAM prime of at least 100 μg RNA can be administered. A SAM prime of at least 30 μg RNA can be administered. A SAM prime of at least 10 μg RNA can be administered. A SAM prime of at least 3 μg RNA can be administered. A SAM prime of less than or equal to 300 μg RNA can be administered. A SAM prime of less than or equal to 100 μg RNA can be administered. For ChAdV68 priming, 1×10¹² or less of viral particles can be administered. For ChAdV68 priming, 3×10¹¹ or less of the viral particles can be administered. For ChAdV68 priming, at least 1×10¹¹ of the viral particles can be administered. For ChAdV68 priming, between 1×10¹¹ and 1×10¹², between 3×10¹¹ and 1×10¹², or between 1×10¹¹ and 3×10¹¹ of the viral particles can be administered. For ChAdV68 priming, 1×10¹¹, 3×10¹¹, or 1×10¹² of the viral particles can be administered. For ChAdV68 priming, the viral particles can be at a concentration of at 5×10¹¹ vp/mL.

A vaccine boost (boosting vaccine) can be injected (e.g., intramuscularly) after prime vaccination. A boosting vaccine can be administered about every 1, 2, 3, 4, 5, 6, 7, 8, 9, or 10 weeks, e.g., every 4 weeks and/or 8 weeks after the prime. A boosting vaccine can be administered 4 weeks after the prime. A boosting vaccine can be administered a month after the prime. A boosting vaccine can be administered 28 days after the prime. A boosting vaccine can be administered 28 days after the prime. A boosting vaccine can be administered 113 days after the prime. A boosting vaccine can be administered at least 4 weeks after the prime. A boosting vaccine can be administered at least a month after the prime. A boosting vaccine can be administered at least 28 days after the prime. A boosting vaccine can be administered at least 28 days after the prime. A boosting vaccine can be administered at least 113 days after the prime. A boosting vaccine can be administered between 28 to 113 days after the prime. Bilateral injections per dose can be used. For example, one or more injections of ChAdV68 (C68) can be used (e.g., total dose 1×10¹² viral particles); one or more injections of self-amplifying RNA (samRNA or SAM), such as a dose of 3 μg, 10 μg, 30 μg, 100 μg, or 300 μg RNA can be used. A SAM boosting dose of 30 μg or less can be used. A SAM boosting dose of 10 μg or less can be used. A SAM boosting dose of 3 μg or less can be used. One or more injections of samRNA at a dose of 30 μg or less can be used. A dose of 30 μg or less can represent the total content of RNA/samRNA administered. A dose of 30 μg or less can represent the total content of RNA/samRNA administered and include only a single distinct samRNA construct. A SAM boost of between 10-30 μg, 10-100 μg, 10-300 μg, 30-100 μg, 30-300 μg, or 100-300 μg RNA can be administered. A SAM boost of between 10-500 μg, 10-1000 μg, 30-500 μg, 30-1000 μg, or 500-1000 μg RNA can be administered. A SAM boost of at least 400 μg, at least 500 μg, at least 600 μg, at least 700 μg, at least 800 μg, at least 900 μg, at least 1000 μg RNA can be administered. A SAM boost of 10 μg, 30 μg, 100 μg, or 300 μg RNA can be administered. A SAM boost of 300 μg RNA can be administered. A SAM boost of 100 μg RNA can be administered. A SAM boost of 30 μg RNA can be administered. A SAM boost of 10 μg RNA can be administered. A SAM boost of 3 μg RNA can be administered. A SAM boost of at least 300 μg RNA can be administered. A SAM boost of at least 100 μg RNA can be administered. A SAM boost of at least 30 μg RNA can be administered. A SAM boost of at least 10 μg RNA can be administered. A SAM boost of at least 3 μg RNA can be administered. A SAM boost of less than or equal to 300 μg RNA can be administered. A SAM boost of less than or equal to 100 μg RNA can be administered. A vaccine boost can include the same antigen cassette (e.g., encode the same SARS-CoV-2 immunogenic polypeptide) as a priming dose. A vaccine boost can include a different antigen cassette as a priming dose. A vaccine boost can include the same composition as a priming dose. As an illustrative non-limiting example, a priming dose can include a non-samRNA based mRNA vaccine (e.g., a commercially available SARS-CoV-2 mRNA vaccine, such as mRNA-1273/SpikeVax® [Moderna]) or other SARS-CoV-2 vaccine platform (e.g., commercially available SARS-CoV-2 vaccine platforms, including, but not limited to, Comirnaty® [BioNTech/Pfizer], AZD1222/Covishield® [Oxford/AstraZeneca], or Ad26.COV2.S/JNJ-78436735 [Janssen/Johnson & Johnson]), and a boosting dose can included any of the samRNA-based and/or ChAdV68-based composition described herein.

A dose of can represent the total content of RNA/samRNA administered. A dose of can represent the total content of RNA/samRNA administered and include only a single distinct samRNA construct.

Immune monitoring can be performed before, during, and/or after vaccine administration. Such monitoring can inform safety and efficacy, among other parameters.

To perform immune monitoring, PBMCs are commonly used. PBMCs can be isolated before prime vaccination, and after prime vaccination (e.g. 4 weeks and 8 weeks). PBMCs can be harvested just prior to boost vaccinations and after each boost vaccination (e.g. 4 weeks and 8 weeks).

Immune responses, such as T cell responses and B cells responses, can be assessed as part of an immune monitoring protocol. For example, the ability of a vaccine composition described herein to stimulate an immune response can be monitored and/or assessed. As used herein, “stimulate an immune response” refers to any increase in a immune response, such as initiating an immune response (e.g., a priming vaccine stimulating the initiation of an immune response in a naïve subject) or enhancement of an immune response (e.g., a boosting vaccine stimulating the enhancement of an immune response in a subject having a pre-existing immune response to an antigen, such as a pre-existing immune response initiated by a priming vaccine). Enhancing an immune response can include stimulating an immune response in a convalescent subject (e.g., a boosting vaccine stimulating the enhancement of an immune response in a convalescent Covid-19 subject). A subject can include an HIV positive subject. T cell responses can be measured using one or more methods known in the art such as ELISpot, intracellular cytokine staining, cytokine secretion and cell surface capture, T cell proliferation, MHC multimer staining, or by cytotoxicity assay. T cell responses to epitopes encoded in vaccines can be monitored from PBMCs by measuring induction of cytokines, such as IFN-gamma, using an ELISpot assay. Specific CD4 or CD8 T cell responses to epitopes encoded in vaccines can be monitored from PBMCs by measuring induction of cytokines captured intracellularly or extracellularly, such as IFN-gamma, using flow cytometry. Specific CD4 or CD8 T cell responses to epitopes encoded in the vaccines can be monitored from PBMCs by measuring T cell populations expressing T cell receptors specific for epitope/MHC class I complexes using MHC multimer staining. Specific CD4 or CD8 T cell responses to epitopes encoded in the vaccines can be monitored from PBMCs by measuring the ex vivo expansion of T cell populations following 3H-thymidine, bromodeoxyuridine and carboxyfluoresceine-diacetate-succinimidylester (CFSE) incorporation. The antigen recognition capacity and lytic activity of PBMC-derived T cells that are specific for epitopes encoded in vaccines can be assessed functionally by chromium release assay or alternative colorimetric cytotoxicity assays.

B cell responses can be measured using one or more methods known in the art such as assays used to determine B cell differentiation (e.g., differentiation into plasma cells), B cell or plasma cell proliferation, B cell or plasma cell activation (e.g., upregulation of costimulatory markers such as CD80 or CD86), antibody class switching, and/or antibody production (e.g., an ELISA).

Vaccination regimens can include assessing neutralizing antibody titers against the vaccine composition of interest, such as a ChAdV68-based vaccine. For example, a ChAdV68-based vaccine can be administered as a priming dose, and re-administration of the ChAdV68-based vaccine as a boosting dose follows determining ChAdV-specific neutralizing antibody titers are below a neutralization threshold prior to re-administration. The neutralizing antibody titer can be an NT50 value calculated as a minimum dilution of sera from the immunized subject that neutralizes a ChAdV virus by 50%. Determining the neutralizing antibody titer can include the steps of: (1) contacting one or more dilutions of sera from the immunized subject with a ChAdV virus under conditions sufficient for neutralization of the ChAdV virus; and (2) assessing neutralization of the ChAdV virus relative to a non-neutralized virus.

### VIII. Isolation and Detection of HLA Peptides

Isolation of HLA-peptide molecules was performed using classic immunoprecipitation (IP) methods after lysis and solubilization of the tissue sample (55-58). Examples and methods are described in more detail in international patent application publication WO/2018/208856, herein incorporated by reference, in its entirety, for all purposes.

### IX. Presentation Model

Presentation models can be used to identify likelihoods of peptide presentation in patients. Various presentation models are known to those skilled in the art, for example the presentation models described in more detail in U.S. Pat. No. 10,055,540, US Application Pub. No. US20200010849A1 and US20110293637, and international patent application publications WO/2018/195357, WO/2018/208856, and WO2016187508, each herein incorporated by reference, in their entirety, for all purposes.

### X. Training Module

Training modules can be used to construct one or more presentation models based on training data sets that generate likelihoods of whether peptide sequences will be presented by MHC alleles associated with the peptide sequences. Various training modules are known to those skilled in the art, for example the presentation models described in more detail in U.S. Pat. No. 10,055,540, US Application Pub. No. US20200010849A1, and international patent application publications WO/2018/195357 and WO/2018/208856, each herein incorporated by reference, in their entirety, for all purposes. A training module can construct a presentation model to predict presentation likelihoods of peptides on a per-allele basis. A training module can also construct a presentation model to predict presentation likelihoods of peptides in a multiple-allele setting where two or more MHC alleles are present.

### XI. Prediction Module

A prediction module can be used to receive sequence data and select candidate antigens in the sequence data using a presentation model. Specifically, the sequence data may be DNA sequences, RNA sequences, and/or protein sequences extracted from infected cells patients or infectious disease organisms themselves (e.g., SARS-CoV-2). A prediction module may identify candidate antigens that are pathogen-derived peptides (e.g., SARS-CoV-2 derived), such as by comparing sequence data extracted from normal tissue cells of a patient with the sequence data extracted from infected cells of the patient to identify portions containing one or more infectious disease organism associated antigens. A prediction module may identify candidate antigens that are expressed in an infected cell or infected tissue in comparison to a normal cell or tissue by comparing sequence data extracted from normal tissue cells of a patient with the sequence data extracted from infected tissue cells of the patient to identify expressed candidate antigens (e.g., identifying expressed polynucleotides and/or polypeptides specific to an infectious disease).

A presentation module can apply one or more presentation model to processed peptide sequences to estimate presentation likelihoods of the peptide sequences. Specifically, the prediction module may select one or more candidate antigen peptide sequences that are likely to be presented on infected cell HLA molecules by applying presentation models to the candidate antigens. In one implementation, the presentation module selects candidate antigen sequences that have estimated presentation likelihoods above a predetermined threshold. In another implementation, the presentation model selects the N candidate antigen sequences that have the highest estimated presentation likelihoods (where N is generally the maximum number of epitopes that can be delivered in a vaccine). A vaccine including the selected candidate antigens for a given patient can be injected into the patient to stimulate immune responses.

### XI.B.Cassette Design Module

### XI.B.1 Overview

A cassette design module can be used to generate a vaccine cassette sequence based on selected candidate peptides for injection into a patient. For example, a cassette design module can be used to generate a sequence encoding concatenated epitope sequences, such as concatenated T cell epitopes. Various cassette design modules are known to those skilled in the art, for example the cassette design modules described in more detail in U.S. Pat. No. 10,055,540, US Application Pub. No. US20200010849A1, and international patent application publications WO/2018/195357 and WO/2018/208856, each herein incorporated by reference, in their entirety, for all purposes.

A set of therapeutic epitopes may be generated based on the selected peptides determined by a prediction module associated with presentation likelihoods above a predetermined threshold, where the presentation likelihoods are determined by the presentation models. However it is appreciated that in other embodiments, the set of therapeutic epitopes may be generated based on any one or more of a number of methods (alone or in combination), for example, based on binding affinity or predicted binding affinity to HLA class I or class II alleles of the patient, binding stability or predicted binding stability to HLA class I or class II alleles of the patient, random sampling, and the like.

Therapeutic epitopes may correspond to selected peptides themselves. Therapeutic epitopes may also include C- and/or N-terminal flanking sequences in addition to the selected peptides. N- and C-terminal flanking sequences can be the native N- and C-terminal flanking sequences of the therapeutic vaccine epitope in the context of its source protein. Therapeutic epitopes can represent a fixed-length epitope Therapeutic epitopes can represent a variable-length epitope, in which the length of the epitope can be varied depending on, for example, the length of the C- or N-flanking sequence. For example, the C-terminal flanking sequence and the N-terminal flanking sequence can each have varying lengths of 2-5 residues, resulting in 16 possible choices for the epitope.

A cassette design module can also generate cassette sequences by taking into account presentation of junction epitopes that span the junction between a pair of therapeutic epitopes in the cassette. Junction epitopes are novel non-self but irrelevant epitope sequences that arise in the cassette due to the process of concatenating therapeutic epitopes and linker sequences in the cassette. The novel sequences of junction epitopes are different from the therapeutic epitopes of the cassette themselves.

A cassette design module can generate a cassette sequence that reduces the likelihood that junction epitopes are presented in the patient. Specifically, when the cassette is injected into the patient, junction epitopes have the potential to be presented by HLA class I or HLA class II alleles of the patient, and stimulate a CD8 or CD4 T-cell response, respectively. Such reactions are often times undesirable because T-cells reactive to the junction epitopes have no therapeutic benefit, and may diminish the immune response to the selected therapeutic epitopes in the cassette by antigenic competition.⁷⁶

A cassette design module can iterate through one or more candidate cassettes, and determine a cassette sequence for which a presentation score of junction epitopes associated with that cassette sequence is below a numerical threshold. The junction epitope presentation score is a quantity associated with presentation likelihoods of the junction epitopes in the cassette, and a higher value of the junction epitope presentation score indicates a higher likelihood that junction epitopes of the cassette will be presented by HLA class I or HLA class II or both.

In one embodiment, a cassette design module may determine a cassette sequence associated with the lowest junction epitope presentation score among the candidate cassette sequences.

A cassette design module may iterate through one or more candidate cassette sequences, determine the junction epitope presentation score for the candidate cassettes, and identify an optimal cassette sequence associated with a junction epitope presentation score below the threshold.

A cassette design module may further check the one or more candidate cassette sequences to identify if any of the junction epitopes in the candidate cassette sequences are self-epitopes for a given patient for whom the vaccine is being designed. To accomplish this, the cassette design module checks the junction epitopes against a known database such as BLAST. In one embodiment, the cassette design module may be configured to design cassettes that avoid junction self-epitopes.

A cassette design module can perform a brute force approach and iterate through all or most possible candidate cassette sequences to select the sequence with the smallest junction epitope presentation score. However, the number of such candidate cassettes can be prohibitively large as the capacity of the vaccine increases. For example, for a vaccine capacity of 20 epitopes, the cassette design module has to iterate through ˜1018 possible candidate cassettes to determine the cassette with the lowest junction epitope presentation score. This determination may be computationally burdensome (in terms of computational processing resources required), and sometimes intractable, for the cassette design module to complete within a reasonable amount of time to generate the vaccine for the patient. Moreover, accounting for the possible junction epitopes for each candidate cassette can be even more burdensome. Thus, a cassette design module may select a cassette sequence based on ways of iterating through a number of candidate cassette sequences that are significantly smaller than the number of candidate cassette sequences for the brute force approach.

A cassette design module can generate a subset of randomly or at least pseudo-randomly generated candidate cassettes, and selects the candidate cassette associated with a junction epitope presentation score below a predetermined threshold as the cassette sequence. Additionally, the cassette design module may select the candidate cassette from the subset with the lowest junction epitope presentation score as the cassette sequence. For example, the cassette design module may generate a subset of ˜1 million candidate cassettes for a set of 20 selected epitopes, and select the candidate cassette with the smallest junction epitope presentation score. Although generating a subset of random cassette sequences and selecting a cassette sequence with a low junction epitope presentation score out of the subset may be sub-optimal relative to the brute force approach, it requires significantly less computational resources thereby making its implementation technically feasible. Further, performing the brute force method as opposed to this more efficient technique may only result in a minor or even negligible improvement in junction epitope presentation score, thus making it not worthwhile from a resource allocation perspective. A cassette design module can determine an improved cassette configuration by formulating the epitope sequence for the cassette as an asymmetric traveling salesman problem (TSP). Given a list of nodes and distances between each pair of nodes, the TSP determines a sequence of nodes associated with the shortest total distance to visit each node exactly once and return to the original node. For example, given cities A, B, and C with known distances between each other, the solution of the TSP generates a closed sequence of cities, for which the total distance traveled to visit each city exactly once is the smallest among possible routes. The asymmetric version of the TSP determines the optimal sequence of nodes when the distance between a pair of nodes are asymmetric. For example, the “distance” for traveling from node A to node B may be different from the “distance” for traveling from node B to node A. By solving for an improved optimal cassette using an asymmetric TSP, the cassette design module can find a cassette sequence that results in a reduced presentation score across the junctions between epitopes of the cassette. The solution of the asymmetric TSP indicates a sequence of therapeutic epitopes that correspond to the order in which the epitopes should be concatenated in a cassette to minimize the junction epitope presentation score across the junctions of the cassette. A cassette sequence determined through this approach can result in a sequence with significantly less presentation of junction epitopes while potentially requiring significantly less computational resources than the random sampling approach, especially when the number of generated candidate cassette sequences is large. Illustrative examples of different computational approaches and comparisons for optimizing cassette design are described in more detail in U.S. Pat. No. 10,055,540, US Application Pub. No. US20200010849A1, and international patent application publications WO/2018/195357 and WO/2018/208856, each herein incorporated by reference, in their entirety, for all purposes.

A cassette design module can also generate cassette sequences by taking into account additional protein sequences encoded in the vaccine. For example, a cassette design module used to generate a sequence encoding concatenated T cell epitopes can take into account T cell epitopes already encoded by additional protein sequences present in the vaccine (e.g., full-length protein sequences), such as by removing T cell epitopes already encoded by the additional protein sequences from the list of candidate sequences.

A cassette design module can also generate cassette sequences by taking into account the size of the sequences. Without wishing to be bound by theory, in general, increased cassette size can negatively impact vaccine aspects, such as vaccine production and/or vaccine efficacy. In one example, the cassette design module can take into account overlapping sequences, such as overlapping T cell epitope sequences. In general, a single sequence containing overlapping T cell epitope sequences (also referred to as a “frame”) is more efficient than separately linking individual T cell epitope sequences as it reduces the sequence size needed to encode the multiple peptides. Accordingly, in an illustrative example, a cassette design module used to generate a sequence encoding concatenated T cell epitopes can take into account the cost/benefit of extending a candidate T cell epitope to encode one or more additional T cell epitopes, such as determining the benefit gained in additional population coverage for an MHC presenting the additional T cell epitope versus the cost of increasing the size of the sequence.

A cassette design module can also generate cassette sequences by taking into account the magnitude of stimulation of an immune response generated by validated epitopes.

A cassette design module can also generate cassette sequences by taking into account presentation of encoded epitopes across a population, for example that at least one immunogenic epitope is presented by at least one HLA across a proportion of a population, for example by at least 85%, 90%, or 95% of a population (e.g., HLA-A, HLA-B and HLA-C genes over four major ethnic groups, namely European (EUR), African American (AFA), Asian and Pacific Islander (APA) and Hispanic (HIS)). As an illustrative non-limiting example, a cassette design module can also generate cassette sequences such that at least one HLA is present at least across 85%, 90%, or 95% of a population that presents at least one validated epitope or presents at least 4, 5, 6, or 7 predicted epitopes.

A cassette design module can also generate cassette sequences by taking into account other aspects that improve potential safety, such as limiting encoding or the potential to encode a functional protein, functional protein domain, functional protein subunit, or functional protein fragment potentially presenting a safety risk. In some cases, a cassette design module can limit sequence size of encoded peptides such that are less than 50%, less than 49%, less than 48%, less than 47%, less than 46%, less than 45%, less than 45%, less than 43%, less than 42%, less than 41%, less than 40%, less than 39%, less than 38%, less than 37%, less than 36%, less than 35%, less than 34%, or less than 33% of the translated, corresponding full-length protein. In some cases, a cassette design module can limit sequence size of encoded peptides such that a single contiguous sequence is less than 50% of the translated, corresponding full-length protein, but more than one sequence may be derived from the same translated, corresponding full-length protein and together encode more than 50%. In an illustrative example, if a single sequence containing overlapping T cell epitope sequences (“frame”) is larger than 50% of the translated, corresponding full-length protein, the frame can be split into multiple frames (e.g., f1, f2 etc.) such that each frame is less than 50% of the translated, corresponding full-length protein. A cassette design module can also limit sequence size of encoded peptides such that a single contiguous sequence is less than 49%, less than 48%, less than 47%, less than 46%, less than 45%, less than 45%, less than 43%, less than 42%, less than 41%, less than 40%, less than 39%, less than 38%, less than 37%, less than 36%, less than 35%, less than 34%, or less than 33% of the translated, corresponding full-length protein. Where multiple frames from the same gene are encoded, the multiple frames can have overlapping sequences with each other, in other words each separately encode the same sequence. Where multiple frames from the same gene are encoded, the two or more nucleic acid sequences derived from the same gene can be ordered such that a first nucleic acid sequence cannot be immediately followed by or linked to a second nucleic acid sequence if the second nucleic acid sequence follows, immediately or not, the first nucleic acid sequence in the corresponding gene. For example, if there are 3 frames within the same gene (f1, f2, f3 in increasing order of amino acid position):
The following cassette orderings are not allowed:
   f1 immediately followed by f2
   f2 immediately followed by f3
   f1 immediately followed by f3
The following cassette orderings are allowed:
   f3 immediately followed by f2
   f2 immediately followed by f1

### XIII. Example Computer

A computer can be used for any of the computational methods described herein. One skilled in the art will recognize a computer can have different architectures. Examples of computers are known to those skilled in the art, for example the computers described in more detail in U.S. Pat. No. 10,055,540, US Application Pub. No. US20200010849A1, and international patent application publications WO/2018/195357 and WO/2018/208856, each herein incorporated by reference, in their entirety, for all purposes.

### XIV. Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### XIV.A. SARS-CoV-2 MHC Epitope Prediction and Vaccine Cassette Construction

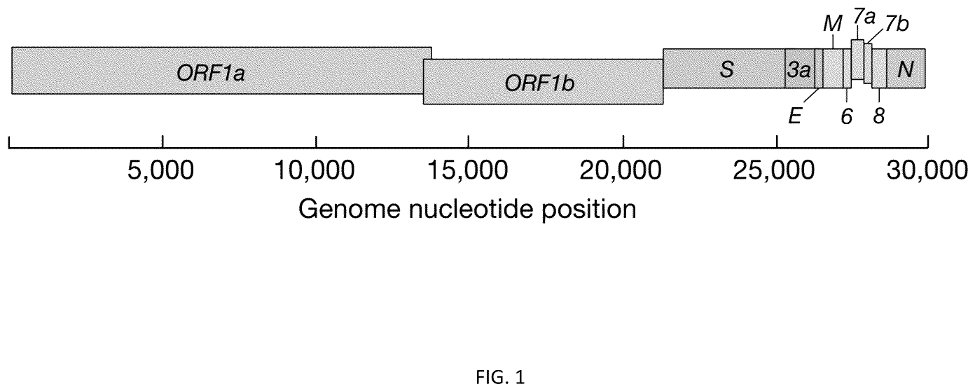

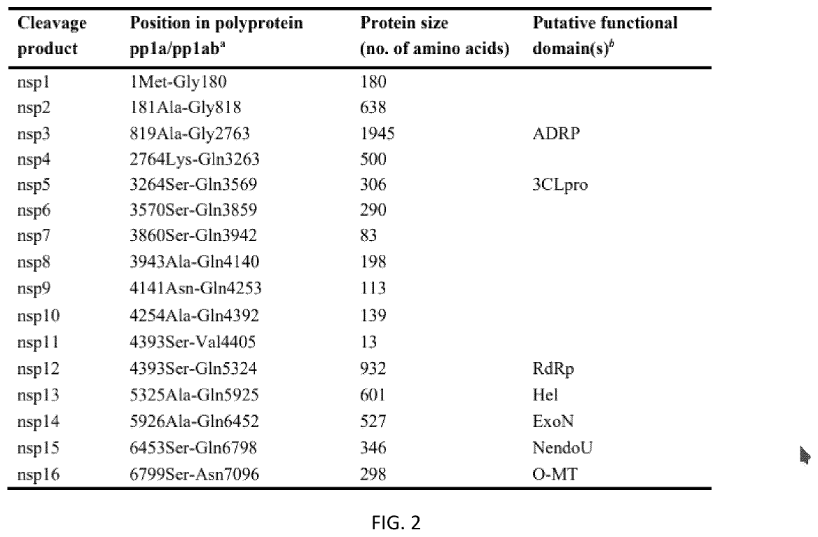

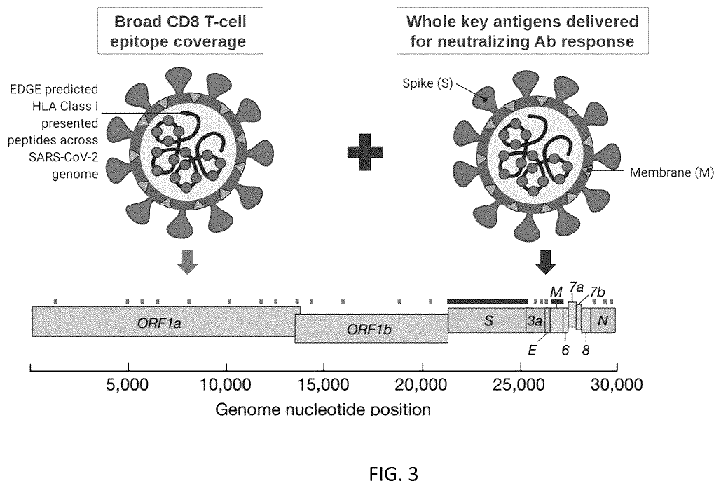

The SARS-CoV-2 belongs to the coronavirus family and its reference genome is a single-stranded RNA sequence of 29,903 base pairs. The genome contains at least 14 open reading frames (ORF) as shown in FIG. 1. Among the encoded genes, the essential genes are replicase ORFlab, spike (S), envelope (E), membrane (M) and nucleocapsid (N). The replicase ORFlab (position 266-21555) encode two proteins namely orf1a and orf1b, the latter is translated by a ribosomal frameshift by −1 at position 13468. The two proteins together contain 16 non-structure proteins (nsp1-nsp16), as depicted in FIG. 2, that is, the ORF1a and ORF1b are cleaved into 16 nsps. The spike protein is thought to bind to the ACE2 receptor of the human cell, allowing the virus to enter the human cell to use its replication machinery to produce and disseminate more copies of the virus.

Because RNA viruses are known to have high mutation rates, a large number of SARS-CoV-2 genomes were analyzed to identify regions in the proteome that are variable. Over 8000 SARS-CoV-2 complete genomes deposited into the GISAID database [www.gisaid.org] as of Apr. 19, 2020 were obtained. Pairwise global alignment of each of the genomes to the SARS-CoV-2 reference genome (Genbank Accession number NC_045512; SEQ ID NO:76) was performed. Sequences on these genomes that are aligned to coding regions of the reference genome were specifically located the. These sequences were then translated to obtain the protein sequences of these SARS-CoV-2. These protein sequences wee then aligned to the respective reference protein sequences to identify variants.

The analysis identified 20 sites on the protein sequences that have a variant rate greater than 1%. These sites are shown in Table 1. In selecting T-cell epitopes, candidate epitopes that cross these variable sites were excluded.

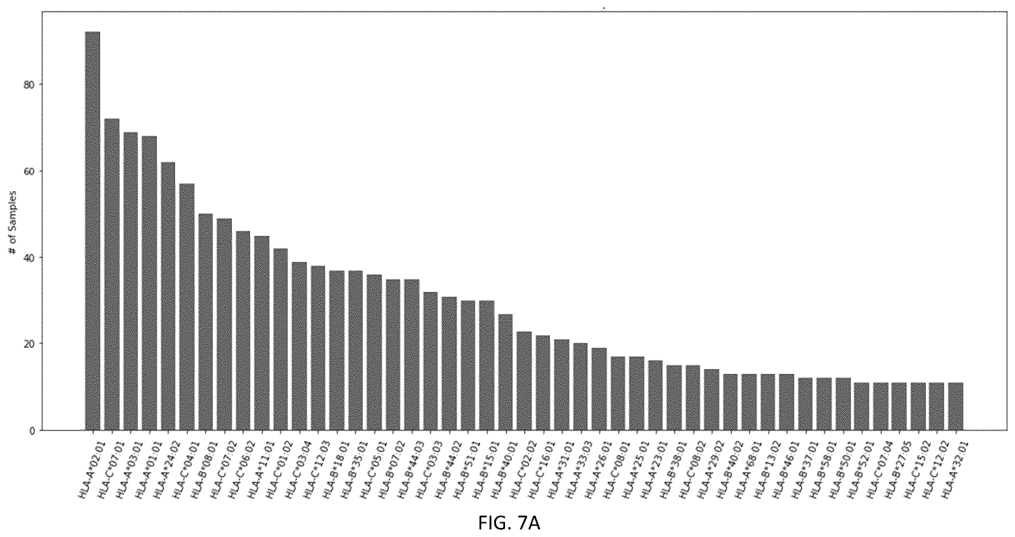

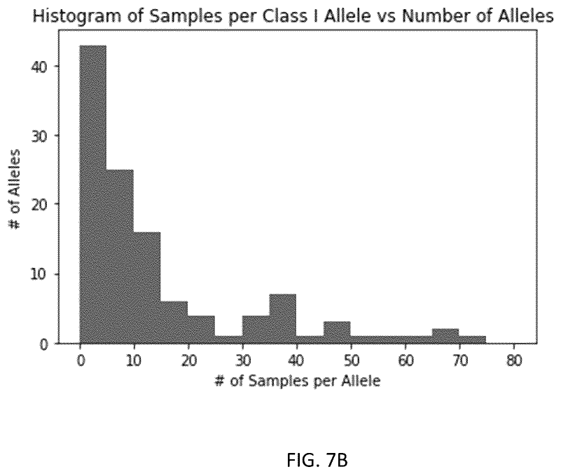

CD8+ epitopes were predicted using our machine learning EDGE platform (see U.S. Pat. No. 10,055,540, herein incorporated by reference for all purposes), which was shown to be best-in-class [Bulik-Sullivan et al. (2018). Deep learning using tumor HLA peptide mass spectrometry datasets improves neoantigen identification. Nature Biotechnology 2018, 37(1), herein incorporated by reference for all purposes]. The model for predicting class I epitopes was recently trained on 507,502 peptides presented in Mass Spectrometry across 398 samples and covers 116 identified alleles, of which 112 alleles (Table 2, FIG. 7) are represented in the haplotype distribution dataset described below.

In order to generate a list of candidate CD8+ T-cell epitopes, the orf1ab protein was split at the cleavage sites shown in FIG. 2. Studies show that the spike protein harbors a furin-like cleavage motif at position 681-684, where the cleavage event occurs following position 684 [Wrapp et al. (2020). Cryo-EM structure of the 2019-nCoV spike in the prefusion conformation. Science, 367(6483), 1260-1263, Ou et al. (2020). Characterization of spike glycoprotein of SARS-CoV-2 on virus entry and its immune cross-reactivity with SARS-CoV. Nature Communications, 11(1), 1620]. The cleavage of the spike protein into S1 and S2 is thought to facilitate the cell entry contributing to the transmissibility of the virus. Accordingly, the Spike protein was split at the furin cleavage site for generating candidate CD8+ T-cell epitopes. All 8-11 mer peptides were then generated from the cleaved proteins and the other proteins, flanked by their native N-terminal and C-terminal 5-mers.

The EDGE machine learning model was run on these candidate epitopes for each HLA class I allele. That is, the presentation score of a candidate epitope is given an EDGE score for each HLA allele. Generally, the probability of a peptide being presented is influenced by the family of the protein containing the peptide, and the expression level of the protein. The EDGE model was also trained on human peptidome datasets. Given there is no equivalent protein family for SARS-CoV-2, for predicting the presentation of a given Sar-CoV-2 peptide, a random protein family was assigned to all peptides. Assigning the same protein family, albeit random, will have the same effect on all SARS-CoV-2 peptides. A high level of expression was also used (tpm=10). A list of candidate epitopes with the EDGE score of 0.001 and above for an HLA allele are shown in Table A, as well as cognate HLA alleles with a predicted EDGE score greater than 0.001, with each cognate pairing ranked as H (EDGE score >0.1), M (EDGE score between 0.01 and 0.1), and L (EDGE score <0.01).

In order to account for the different levels of expression of SARS-CoV-2 genes, the ratio of reported T-cell responses among genes from SARS-CoV-2 genome [Li et al. (2008) T Cell Responses to Whole SARS Coronavirus in Humans. The Journal of Immunology, 181(8), 5490-5500] was used as a proxy for the ratio of gene expression levels. The score of all epitopes from a SARS-CoV-2 gene was then scaled so that the ratio between the 99^{th} percentile of the epitopes in the selected gene and the 99^{th} percentile of the epitopes in the Spike gene followed the ratio reported in [Li et al. (2008). T Cell Responses to Whole SARS Coronavirus in Humans. The Journal of Immunology, 181(8), 5490-5500].

A set of candidate CD8+ epitopes was then selected by choosing those with the scaled EDGE score greater than or equal to a threshold of t=0.01. The threshold was selected from analysis of an HIV LANL dataset (data not shown) so that PPV for T-cell epitopes estimated to be 0.2 and recall is 0.5. The set sequences that are >=90% homologous to known SARs-CoV T-cell epitopes reported in IEDB [Vita et al. (2019). The Immune Epitope Database (IEDB): 2018 update. Nucleic Acids Research, 47(D1), D339-D343.] was also included similar to the approach described in Grifoni et al. [(2020). A Sequence Homology and Bioinformatic Approach Can Predict Candidate Targets for Immune Responses to SARS-CoV-2. Cell Host& Microbe, 27(4), 671-680.e2].

The set of candidate epitopes excluded those sequences that contained at least one of the sites that have a variable rate greater than 0.01, as mentioned above and shown in Table 1.

In order to maximize the coverage of the vaccine over the world population, allele frequencies of HLA-A, HLA-B and HLA-C genes over four major ethnic groups, namely European (EUR), African American (AFA), Asian and Pacific Islander (APA) and Hispanic (HIS) were obtained from the publicly available National Marrow Donor Program dataset [bioinformatics.bethematchclinical.org/hla-resources/haplotype-frequencies/high-resolution-hla-alleles-and-haplotypes-in-the-us-population]. Simulations were then performed to estimate the frequencies of the haplotypes made up by combination of these HLA alleles.

Cassette optimization proceeded as follows:

### Epitope Selection Definitions

Candidate epitope set E: set of candidate CD8+ epitopes with scaled EDGE score greater than or equal to a threshold of t=0.01
Population coverage criteria P: For each of the four ethnicity groups as described above (EUR/AFA/APA/HIS), 95% of the simulated people in that ethnic group have at least 30 candidate epitopes presented in their diplotype
Solution frame set F—all amino acid ranges in the current solution encapsulating the added candidate epitopes
   For each epitope added to the solution, the epitope and 5 flanking native amino acids on each end must be fully contained in a frame of F
   Each frame spans only protein region (including individual NSPs in orf1ab)

### Epitope Selection Method

Population coverage criteria P starts as calculated with all epitopes in the whole gene(s) initially added
If the population coverage criteria P is not satisfied, continually choose the amino acid frame f across SARS-CoV-2 proteome that most efficiently maximizes progress to P
   Defined as the highest ratio of additional population coverage C/additional amino acid bases added AA. (C/AA).
   All candidate frames are either a new 25aa frame (AA=25), or can overlap with frames in the existing solution F—in which case it can add any amount less than 25aa (AA<25)
   Additional population coverage C is the increase in epitope count from E for haplotypes with <20 covered epitopes, weighted (multiplied) by the haplotype's population frequency summed across all four ethnic groups
   20 epitopes per haplotype is determined (experimentally chosen) to be an efficient proxy towards reaching the overall coverage criteria of 30 candidate epitopes per diplotype
   Add f to solution frame set F. Remove from E, candidate epitopes within f.
After frames are chosen, create the final set F:
   First merge overlapping frames, yielding contiguous sequences (i.e. epitope “hotspots”)
   Next ensure all frames are less than 50% of that frame's overall gene size. If frames are larger than this size, divide them into smaller (potentially overlapping) frames that are each smaller than this requirement. Additional more stringent size limitations can be tested
To illustrates the marginal value of larger cassette sizes, frame selection can continue past when P is satisfied—but does not affect the composition of the chosen cassette for the criteria P.

### Cassette Ordering

The frames in solution frame set F are ordered to minimize the EDGE score of junction epitopes (unintended epitopes not part of the solution, created by adjacent frames). Successive frames within a gene are also forbidden to immediately follow each other in the cassette (intra-gene restriction). In other words, intra-gene restriction requires that if there are two or more SARS-CoV-2 derived nucleic acid sequences encoding epitopes derived from the same SARS-CoV-2 gene, the two sequences are ordered such that a first nucleic acid sequence cannot be immediately followed by or linked to a second nucleic acid sequence if the second nucleic acid sequence follows first nucleic acid sequence in the corresponding SARS-CoV-2 gene. For example, if there are 3 frames within the same gene (f1, f2, f3 in increasing order of amino acid position)
The following cassette orderings are impossible:
   f1 immediately followed by f2
   f2 immediately followed by f3
   f1 immediately followed by f3
The following cassette orderings are possible:
   f3 immediately followed by f2
   f2 immediately followed by f1

### Cassette Ordering Method

Google optimization routing tools [developers.google.com/optimization/routing] are used to perform a traveling salesman optimization route, where the distance between each pair of frames in F is:
Infinite, if the frames are not allowed to follow each other in this order, according to the above intra-gene restriction
Otherwise the sum of junction epitope EDGE scores across all alleles, weighted by allele frequency in population

Route finding of the minimal path distance yields the optimal ordering of the frames in the cassette to minimize junctional epitopes and avoid successive frames within a gene.

### Results

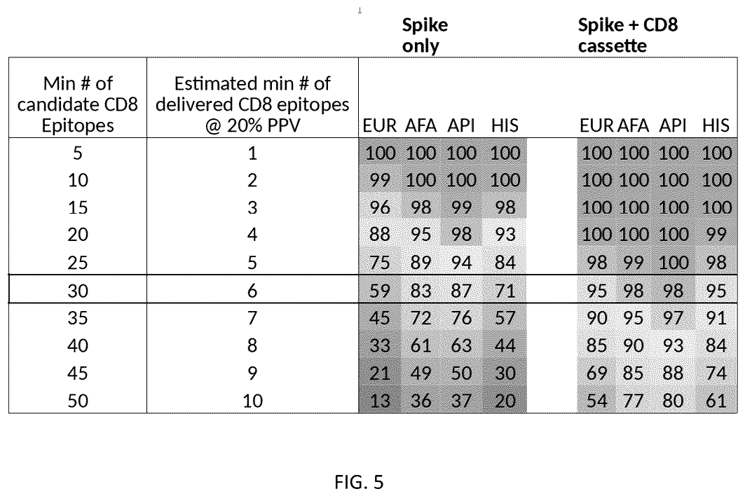

The population coverage criteria P was calculated with all initial epitopes provided by the SARS-CoV-2 Spike protein (SEQ ID NO:59) split into S1 and S2. Applying the optimization algorithms above yielded a 594 amino acid cassette sequence having 18 epitope-encoding frames, as shown in Table 3A. Table C presents each of the additional epitopes contained in the cassette (not including the epitopes derived from the Spike protein). Empirically, the optimal frame set F was produced when the size threshold for all frames was set to less than 42% of that frame's overall gene size. The coverage of the designed cassette over four populations is shown in FIG. 5, with the first column providing the number of SARS-CoV-2 epitopes predicted to be presented and the second column providing the expected number of presented epitopes, based on a 0.2 PPV. Each row shows the protection coverage of each population if a certain number of epitopes is used.

Potential HLA-DRB, HLA-DQ, and HLA-DP MHC class II epitopes from the SARS-CoV-2 proteome were also predicted. The method described for generating candidate CD8/MHC class I epitopes was used to generate peptides with sizes between 9 and 20 amino acids. EDGE model was run for class II to compute EDGE score of each of these peptides against each identifiable allele (see, e.g., U.S. application Ser. No. 16/606,577 and international patent application PCT/US2020/021508, each herein incorporated by reference in their entirety for all purposes). The list of CD4 epitopes with EDGE score greater than 0.001 are presented in Table B, as well as cognate HLA alleles with a predicted EDGE score greater than 0.001, with each cognate pairing ranked as H (EDGE score >0.1), M (EDGE score between 0.01 and 0.1), and L (EDGE score <0.01). HLA-DQ and HLA-DP are referred to by their alpha and beta chains used in the analysis, while HLA-DR is referred to by its beta chain as the alpha chain is generally invariable in the human population, with HLA-DR peptide contact regions particularly invariant.

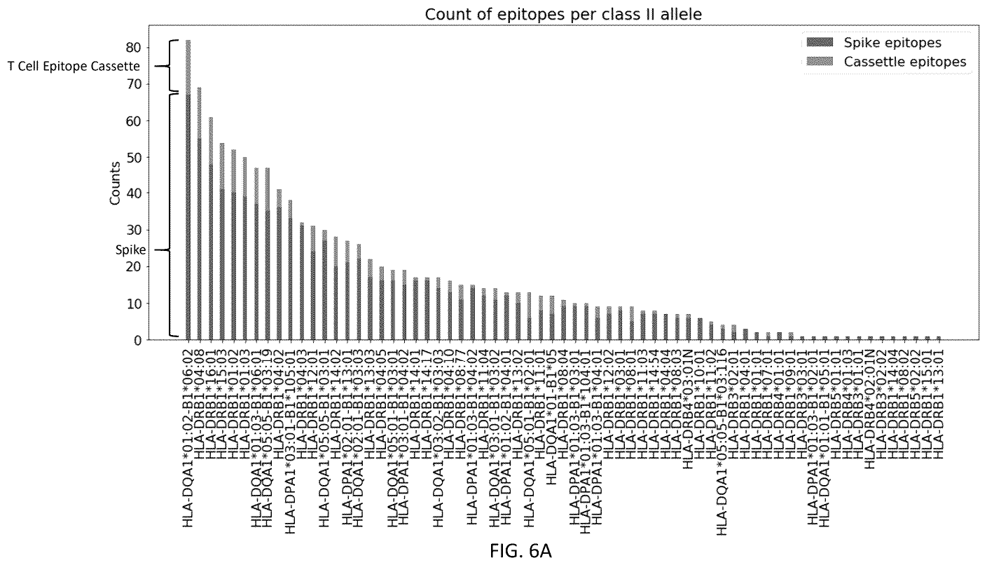

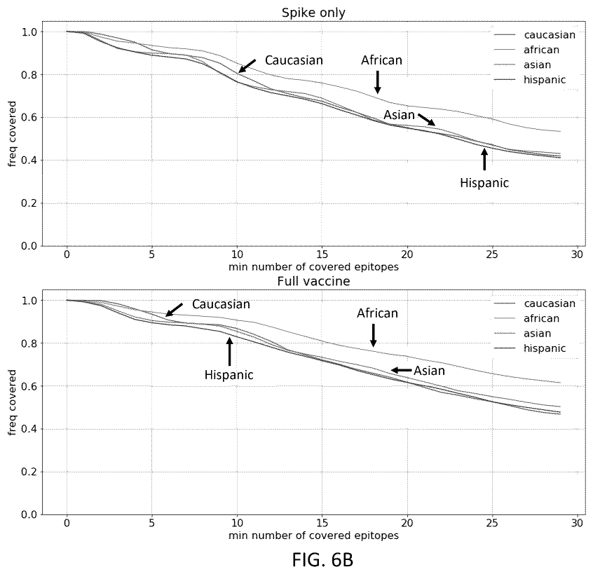

The peptides receiving a score of >0.02 contained in the optimized MHC I cassette frames determined above were then identified. The threshold of 0.02 was chosen because the model prediction has the PPV of 0.2 in predicting Mass Spectrometry data with prevalence ratio positive vs negatives of 1:100. FIG. 6A illustrates the number of predicted epitopes presented by each MHC class II allele examined. FIG. 6B shows the population coverage of MHC class II at the diploid level.

Additional cassettes are designed using the epitope prediction and frame ordering algorithms described above where the initial population coverage criteria P is calculated with all initial epitopes provided by SARS-CoV-2 Membrane (SEQ ID NO:61), SARS-CoV-2 Nucleocapsid (SEQ ID NO:62), SARS-CoV-2 Envelope (SEQ ID NO:63), or combinations (including combinations with SARS-CoV-2 spike) or sequence variants thereof.

TABLE 1
Identified SARS-CoV-2 Mutations (>1%)

| | | Reference | Alternate | Variable |
|---|---|---|---|---|
| Gene | Position | Amino Acid | Amino Acid | Rate |
| orf1ab | 265 | T | I | 0.12474 |
| orflab | 378 | V | I | 0.022349 |
| orflab | 392 | G | D | 0.015073 |
| orflab | 448 | D | deletion | 0.030146 |
| orflab | 739 | I | V | 0.011435 |
| orflab | 765 | P | S | 0.012474 |
| orflab | 876 | A | T | 0.015073 |
| orflab | 3353 | K | R | 0.023909 |
| orflab | 3606 | L | F | 0.130977 |
| orflab | 4715 | P | L | 0.448025 |
| orf1ab | 5828 | P | L | 0.193867 |
| orf1ab | 5865 | Y | C | 0.199584 |
| S | 614 | D | G | 0.439815 |
| ORF3a | 57 | Q | H | 0.150943 |
| ORF3a | 251 | G | V | 0.086003 |
| M | 3 | D | G | 0.013508 |
| M | 175 | T | M | 0.051416 |
| ORF8 | 84 | L | S | 0.267563 |
| N | 203 | R | K | 0.124507 |
| N | 204 | G | R | 0.124068 |

TABLE 2
List of Identifiable Class I alleles

| | | | | | | |
|---|---|---|---|---|---|---|
| HLA- | HLA- | HLA- | HLA- | HLA- | HLA- | HLA- |
| A*01:01 | A*25:01 | A*68:01 | B*15:13 | B*40:06 | B*55:02 | C*07:02 |
| HLA- | HLA- | HLA- | HLA- | HLA- | HLA- | HLA- |
| A*02:01 | A*26:01 | A*68:02 | B*18:01 | B*41:02 | B*56:01 | C*07:04 |
| HLA- | HLA- | HLA- | HLA- | HLA- | HLA- | HLA- |
| A*02:02 | A*26:02 | A*74:01 | B*27:02 | B*42:01 | B*57:01 | C*07:27 |
| HLA- | HLA- | HLA- | HLA- | HLA- | HLA- | HLA- |
| A*02:03 | A*26:03 | B*07:02 | B*27:05 | B*44:02 | B*57:03 | C*08:01 |
| HLA- | HLA- | HLA- | HLA- | HLA- | HLA- | HLA- |
| A*02:04 | A*29:01 | B*07:04 | B*35:01 | B*44:03 | B*58:01 | C*08:02 |
| HLA- | HLA- | HLA- | HLA- | HLA- | HLA- | HLA- |
| A*02:05 | A*29:02 | B*07:05 | B*35:02 | B*44:05 | B*58:02 | C*08:03 |
| HLA- | HLA- | HLA- | HLA- | HLA- | HLA- | HLA- |
| A*02:06 | A*30:01 | B*08:01 | B*35:03 | B*45:01 | C*01:02 | C*12:02 |
| HLA- | HLA- | HLA- | HLA- | HLA- | HLA- | HLA- |
| A*02:07 | A*30:02 | B*13:01 | B*35:08 | B*46:01 | C*02:02 | C*12:03 |
| HLA- | HLA- | HLA- | HLA- | HLA- | HLA- | HLA- |
| A*02:11 | A*31:01 | B*13:02 | B*35:12 | B*48:01 | C*03:02 | C*12:05 |
| HLA- | HLA- | HLA- | HLA- | HLA- | HLA- | HLA- |
| A*03:01 | A*32:01 | B*14:01 | B*37:01 | B*49:01 | C*03:03 | C*14:02 |
| HLA- | HLA- | HLA- | HLA- | HLA- | HLA- | HLA- |
| A*03:02 | A*33:01 | B*14:02 | B*38:01 | B*50:01 | C*03:04 | C*14:03 |
| HLA- | HLA- | HLA- | HLA- | HLA- | HLA- | HLA- |
| A*11:01 | A*33:03 | B*15:01 | B*38:02 | B*51:01 | C*04:01 | C*15:02 |
| HLA- | HLA- | HLA- | HLA- | HLA- | HLA- | HLA- |
| A*11:02 | A*34:01 | B*15:02 | B*39:01 | B*52:01 | C*04:03 | C*15:05 |
| HLA- | HLA- | HLA- | HLA- | HLA- | HLA- | HLA- |
| A*23:01 | A*34:02 | B*15:03 | B*39:06 | B*53:01 | C*05:01 | C*16:01 |
| HLA- | HLA- | HLA- | HLA- | HLA- | HLA- | HLA- |
| A*24:02 | A*36:01 | B*15:10 | B*40:01 | B*54:01 | C*06:02 | C*16:04 |
| HLA- | HLA- | HLA- | HLA- | HLA- | HLA- | HLA- |
| A*24:07 | A*66:01 | B*15:11 | B*40:02 | B*55:01 | C*07:01 | C*17:01 |

TABLE 3A
Cassette Epitope Frames in Conjunction with Spike Protein

| | | | | Amino Acid |
|---|---|---|---|---|
| Frame | Gene | Gene Start | Gene End | Length |
| 1 | M | 172 | 204 | 33 |
| 2 | orf1ab | 4154 | 4178 | 25 |
| 3 | N | 301 | 345 | 45 |
| 4 | N | 151 | 175 | 25 |
| 5 | N | 71 | 95 | 25 |
| 6 | ORF3a | 106 | 130 | 25 |
| 7 | orf1ab | 4419 | 4443 | 25 |
| 8 | N | 259 | 283 | 25 |
| 9 | orf1ab | 5371 | 5395 | 25 |
| 10 | M | 85 | 140 | 56 |
| 11 | N | 352 | 393 | 42 |
| 12 | orf1ab | 2580 | 2604 | 25 |
| 13 | ORF3a | 1 | 47 | 47 |
| 14 | M | 34 | 60 | 27 |
| 15 | ORF3a | 53 | 86 | 34 |
| 16 | orf1ab | 2794 | 2818 | 25 |
| 17 | ORF3a | 199 | 255 | 57 |
| 18 | E | 44 | 71 | 28 |

### XIV.B. SARS-CoV-2 Vaccine Design

A series of vaccines against SARS-CoV-2 were designed to produce a balanced immune response inducing both neutralizing antibodies (from B cells) as well as effector and memory CD8+ T cell responses for maximum efficacy. In general, neutralizing antibodies to viral surface proteins can serve to prevent viral entry into cells and virus epitope-specific CD8+ T cells kill virally-infected cells. In addition, vaccines against SARS-CoV-2 were designed to maximize the coverage of the vaccine across the world population, i.e., target most individuals (e.g., >95%) receive a large number (e.g., >=30) of candidate CD8+ epitopes across all major ancestry groups while minimizing our cassette sequence footprint.

### Antigens and Cassettes

Vaccines are constructed encoding the MHC epitope-encoding cassettes designed using the epitope prediction and frame ordering algorithms described above. An exemplary cassette (herein referred to as the Concatenated EDGE predicted SARS-CoV-2 MHC Class I Epitope Cassette or EDGE Predicted Epitopes (EPE)) was generated where the initial population coverage criteria P was calculated with all initial epitopes provided by SARS-CoV-2 Spike, as described above.

Vaccines are also designed encoding various full-length proteins, either alone or in combination, generally for the purposes of stimulating a B cell response. Full-length proteins include SARS-CoV-2 Spike (SEQ ID NO:59), SARS-CoV-2 Membrane (SEQ ID NO:61), SARS-CoV-2 Nucleocapsid (SEQ ID NO:62), and SARS-CoV-2 Envelope (SEQ ID NO:63), sequences of which are shown in Table 3B.

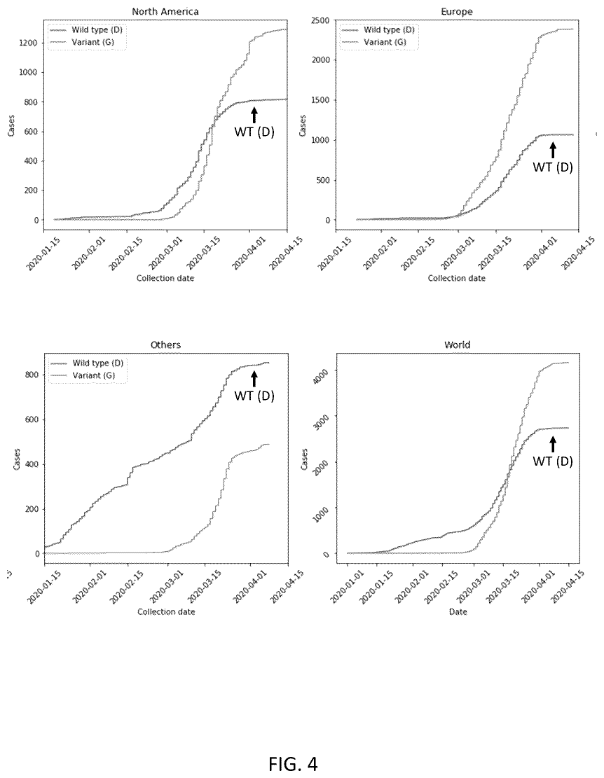

With respect to the Spike protein, initial analysis of prevalent SARS-CoV-2 variants (as described above, see Table 1) identified a Spike protein variant present in almost 44% of genomes. Subsequent analysis of the over 8000 SARS-CoV-2 complete genomes identified a dominant variant at position 614 where the wildtype amino acid aspartic (D) is mutated to glycine (G). The mutation, denotated as D614G, is found on 60.05% of genomes sequenced worldwide, and 70.46% and 58.49% of the sequences in Europe and North America, respectively (FIG. 4). Accordingly, Spike proteins are used that contain the prevalent D614G Spike variant, with reference to the reference Spike protein (SEQ ID NO:59). Omicron SARS-CoV-2 variants assessed include the D614G mutation. In addition, a modified Spike protein was engineered to bias the Spike protein to remain in a predominantly prefusion state, as the prefusion Spike state may be a better target for antibody-mediated neutralization of the virus. The following mutations were selected: R682V to disrupt the Furin cleavage site; R815N to disrupt cleavage site within S2, and K986P and V987P to interfere with the secondary structure of Spike. Accordingly, “modified” Spike proteins are used that contain one or more of the following mutations, with reference to the reference Spike protein (SEQ ID NO:59): a D614G mutation, a R682V mutation, a R815N mutation, a K986P mutation, or a V987P mutation. For reference, a modified Spike proving having all of the Spike mutations is shown in SEQ ID NO:60. Furin cleavage sites can also be disrupted by one or more (or each of) mutations to R682, R683, and R685, such as mutating 682-685 RRAR (SEQ ID NO: 125) to GSAS (SEQ ID NO: 126). Corresponding Omicron/B.1.1.529 Furin mutations are R679, R680, and R682; corresponding Omicron/B.1.1.529 BA5 subvariant Furin mutations are R677, R678, and R680. Corresponding Omicron/B.1.1.529 mutations that interfere with the secondary structure of Spike are K983P and V984P; corresponding Omicron/B.1.1.529 BA5 subvariant mutations that interfere with the secondary structure of Spike are K981P and V982P. Mutations to Omicron/B.1.1.529 are with reference to the native Omicron/B.1.1.529 Spike protein (SEQ ID NO:27977). Mutations to Omicron/B.1.1.529 BA5 subvariant are with reference to the native Omicron/B.1.1.529 BA5 subvariant Spike protein (SEQ ID NO:27979).

Various vaccine designs and their respective cassette nucleotide sequences are presented in more detail in Table 4. For SAM based vaccines, promoter and/or poly(A) signal sequences can be removed given cassettes are generally operably linked to the endogenous 26S promoter and poly(A) sequence provided by the vector backbone. Depending on the cassette features and configuration, translated proteins (e.g., those in Table 3B) may also include an additional sequence(s) related to the particular expression strategy, such as a 2A ribosome skipping sequence elements (or fragments thereof following translation) and additional 26S promoter sequences.

An antigen cassette including an Omicron/B.1.1.529 Spike variant was constructed (“SAM-Nuc-TCE11-SpikeB.1.1.529” SEQ ID NO: 27976) that includes: SARS-CoV-2 Spike protein encoded by Cool Tool optimized sequence version 1 (SEQ ID NO:79) with 37 Omicron mutations manually changed as applicable (see SEQ ID NO:27977); mutations disrupting the Furin cleavage site (679-682 RRAR [SEQ ID NO: 125] to GSAS [SEQ ID NO: 126]); and K983P and V984P to interfere with the secondary structure of Spike, with reference to the native Omicron Spike protein (SEQ ID NO:27977). An antigen cassette including an Omicron/B.1.1.529 BA5 subvariant Spike was constructed through replacing the nucleotide sequences encoding the Omicron/B.1.1.529 Spike in SEQ ID NO: 27976 with nucleotide sequences encoding a Omicron/B.1.1.529 BA5 subvariant Spike.

TABLE 3B
SARS-CoV-2 Proteins

| | | SEQ ID |
|---|---|---|
| Peptide | Amino Acid Sequence | NO: |
| Concatenated | TSRTLSYYKLGASQRVAGDSGFAAYSRYRIGNYCAAGTTQTACTDDNA | 57 |
| EDGE predicted | LAYYNTTKGGWPQIAQFAPSASAFFGMSRIGMEVTPSGTWLTYTGAIK | |
| SARS-CoV-2 | LDDKDPNPANNAAIVLQLPQGTTLPKGFYAEGGVPINTNSSPDDQIG | |
| MHC Class I | YYRRATRRIRLYLYALVYFLQSINFVRIIMRLWLCSTDVVYRAFDIY | |
| Epitope Cassette | NDKVAGFAKFLKTRQKRTATKAYNVTQAFGRRGPEQTQPYVCNAPG | |
| | CDVTDVTQLYLGGMSYYACLVGLMWLSYFIASFRLFARTRSMWSFN | |
| | PETNILLNVPLHGTILTRPLLESELVILLNKHIDAYKTFPPTEPKK | |
| | DKKKKADETQALPQRQKKQQTVTVGDSAEVAVKMFDAYVNTFSSTF | |
| | NVMDLFMRIFTIGTVTLKQGEIKDATPSDFVRATATIPIQASLPFG | |
| | WLILLQFAYANRNRFLYIIKLIFLWLLWPVLAVFQSASKIITLKKR | |
| | WQLALSKGVHFVCNLLLLHVMSKHTDFSSEIIGYKAIDGGVTRDCV | |
| | VLHSYFTSDYYQLYSTQLSTDTGVEHVTFFIYNKIVDEPEEHVQIH | |
| | TIDGSSGVCNIVNVSLVKPSFYVYSRVKNLNSSRVP | |
| Concatenated | MAGTSRTLSYYKLGASQRVAGDSGFAAYSRYRIGNYCAAGTTQTACT | 58 |
| EDGE predicted | DDNALAYYNTTKGGWPQIAQFAPSASAFFGMSRIGMEVTPSGTWLTY | |
| SARS-CoV-2 | TGAIKLDDKDPNPANNAAIVLQLPQGTTLPKGFYAEGGVPINTNSSP | |
| MHC Class I | DDQIGYYRRATRRIRLYLYALVYFLQSINFVRIIMRLWLCSTDVVYR | |
| Epitope Cassette | AFDIYNDKVAGFAKFLKTRQKRTATKAYNVTQAFGRRGPEQTQPYVC | |
| with N-term | NAPGCDVTDVTQLYLGGMSYYACLVGLMWLSYFIASFRLFARTRSMW | |
| leader (bold) | SFNPETNILLNVPLHGTILTRPLLESELVILLNKHIDAYKTFPPTEP | |
| and C-term | KKDKKKKADETQALPQRQKKQQTVTVGDSAEVAVKMFDAYVNTFSST | |
| Universal MHC | FNVMDLFMRIFTIGTVTLKQGEIKDATPSDFVRATATIPIQASLPFG | |
| Class II with | WLILLQFAYANRNRFLYIIKLIFLWLLWPVLAVFQSASKIITLKKRW | |
| GPGPG linkers | QLALSKGVHFVCNLLLLHVMSKHTDFSSEIIGYKAIDGGVTRDCVVL | |
| (SEQ ID NO: 56) | HSYFTSDYYQLYSTQLSTDTGVEHVTFFIYNKIVDEPEEHVQIHTID | |
| (bold italic) | GSSGVCNIVNVSLVKPSFYVYSRVKNLNSSRVPGPGPGAKFVAAWTL | |
| | KAAAGPGPGQYIKANSKFIGITELGPGPG | |
| SARS-CoV-2 | MFVFLVLLPLVSSQCVNLTTRTQLPPAYTNSFTRGVYYPDKVFRSSVL | 59 |
| Spike | HSTQDLFLPFFSNVTWFHAIHVSGTNGTKRFDNPVLPFNDGVYFASTE | |
| | KSNIIRGWIFGTTLDSKTQSLLIVNNATNVVIKVCEFQFCNDPFLGVYY | |
| | HKNNKSWMESEFRVYSSANNCTFEYVSQPFLMDLEGKQGNFKNLREFV | |
| | FKNIDGYFKIYSKHTPINLVRDLPQGFSALEPLVDLPIGINITRFQTL | |
| | LALHRSYLTPGDSSSGWTAGAAAYYVGYLQPRTFLLKYNENGTITDAV | |
| | DCALDPLSETKCTLKSFTVEKGIYQTSNFRVQPTESIVRFPNITNLCP | |
| | FGEVFNATRFASVYAWNRKRISNCVADYSVLYNSASFSTFKCYGVSPT | |
| | KLNDLCFTNVYADSFVIRGDEVRQIAPGQTGKIADYNYKLPDDFTGCV | |
| | IAWNSNNLDSKVGGNYNYLYRLFRKSNLKPFERDISTEIYQAGSTPCN | |
| | GVEGFNCYFPLQSYGFQPTNGVGYQPYRVVVLSFELLHAPATVCGPK | |
| | KSTNLVKNKCVNFNFNGLTGTGVLTESNKKFLPFQQFGRDIADTTDAV | |
| | RDPQTLEILDITPCSFGGVSVITPGTNTSNQVAVLYQDVNCTEVPVAI | |
| | HADQLTPTWRVYSTGSNVFQTRAGCLIGAEHVNNSYECDIPIGAGICA | |
| | SYQTQTNSPRRARSVASQSIIAYTMSLGAENSVAYSNNSIAIPTNFTI | |
| | SVTTEILPVSMTKTSVDCTMYICGDSTECSNLLLQYGSFCTQLNRALT | |
| | GIAVEQDKNTQEVFAQVKQIYKTPPIKDFGGFNFSQILPDPSKPSKRS | |
| | FIEDLLFNKVTLADAGFIKQYGDCLGDIAARDLICAQKFNGLTVLPPL | |
| | LTDEMIAQYTSALLAGTITSGWTFGAGAALQIPFAMQMAYRENGIGVT | |
| | QNVLYENQKLIANQFNSAIGKIQDSLSSTASALGKLQDVVNQNAQALN | |
| | TLVKQLSSNFGAISSVLNDILSRLDKVEAEVQIDRLITGRLQSLQTYV | |
| | TQQLIRAAEIRASANLAATKMSECVLGQSKRVDFCGKGYHLMSFPQSA | |
| | PHGVVFLHVTYVPAQEKNFTTAPAICHDGKAHFPREGVFVSNGTHWFV | |
| | TQRNFYEPQIITTDNTFVSGNCDVVIGIVNNTVYDPLQPELDSFKEEL | |
| | DKYFKNHTSPDVDLGDISGINASVVNIQKEIDRLNEVAKNLNESLIDL | |
| | QELGKYEQYIKWPWYIWLGFIAGLIAIVMVTIMLCCMTSCCSCLKGCC | |
| | SCGSCCKFDEDDSEPVLKGVKLHYT | |
| SARS-CoV-2 | MFVFLVLLPLVSSQCVNLTTRTQLPPAYTNSFTRGVYYPDKVFRSSVL | 60 |
| Modified Spike | HSTQDLFLPFFSNVTWFHAIHVSGTNGTKRFDNPVLPFNDGVYFASTE | |
| (Potential | KSNIIRGWIFGTTLDSKTQSLLIVNNATNVVIKVCEFQFCNDPFLGVY | |
| mutations in | YHKNNKSWMESEFRVYSSANNCTFEYVSQPFLMDLEGKQGNFKNLREF | |
| Bold italic | VFKNIDGYFKIYSKHTPINLVRDLPQGFSALEPLVDLPIGINITRFQT | |
| lowercase. | LLALHRSYLTPGDSSSGWTAGAAAYYVGYLQPRTFLLKYNENGTITDA | |
| modifications | VDCALDPLSETKCTLKSFTVEKGIYQTSNFRVQPTESIVRFPNITNLC | |
| may be in | PFGEVFNATRFASVYAWNRKRISNCVADYSVLYNSASFSTFKCYGVSP | |
| separate or | TKLNDLCFTNVYADSFVIRGDEVRQIAPGQTGKIADYNYKLPDDFTGC | |
| combined | VIAWNSNNLDSKVGGNYNYLYRLFRKSNLKPFERDISTEIYQAGSTPC | |
| constructs) | NGVEGFNCYFPLQSYGFQPTNGVGYQPYRVVVLSFELLHAPATVCGPK | |
| | KSTNLVKNKCVNFNFNGLTGTGVLTESNKKFLPFQQFGRDIADTTDAV | |
| | RDPQTLEILDITPCSFGGVSVITPGTNTSNQVAVLYQgVNCTEVPVAI | |
| | HADQLTPTWRVYSTGSNVFQTRAGCLIGAEHVNNSYECDIPIGAGICA | |
| | SYQTQTNSPvRARSVASQSIIAYTMSLGAENSVAYSNNSIAIPTNFTI | |
| | SVTTEILPVSMTKTSVDCTMYICGDSTECSNLLLQYGSFCTQLNRALT | |
| | GIAVEQDKNTQEVFAQVKQIYKTPPIKDFGGFNFSQILPDPSKPSKnS | |
| | FIEDLLFNKVTLADAGFIKQYGDCLGDIAARDLICAQKFNGLTVLPPL | |
| | LTDEMIAQYTSALLAGTITSGWTFGAGAALQIPFAMQMAYRENGIGVT | |
| | QNVLYENQKLIANQFNSAIGKIQDSLSSTASALGKLQDVVNQNAQALN | |
| | TLVKQLSSNFGAISSVLNDILSRLDppEAEVQIDRLITGRLQSLQTYV | |
| | TQQLIRAAEIRASANLAATKMSECVLGQSKRVDFCGKGYHLMSFPQSA | |
| | PHGVVFLHVTYVPAQEKNFTTAPAICHDGKAHFPREGVFVSNGTHWFV | |
| | TQRNFYEPQIITTDNTFVSGNCDVVIGIVNNTVYDPLQPELDSFKEEL | |
| | DKYFKNHTSPDVDLGDISGINASVVNIQKEIDRLNEVAKNLNESLIDL | |
| | QELGKYEQYIKWPWYIWLGFIAGLIAIVMVTIMLCCMTSCCSCLKGCC | |
| | SCGSCCKFDEDDSEPVLKGVKLHYT | |
| SARS-CoV-2 | MADSNGTITVEELKKLLEQWNLVIGFLFLTWICLLQFAYANRNRFLYI | 61 |
| Membrane | IKLIFLWLLWPVTLACFVLAAVYRINWITGGIAIAMACLVGLMWLSYF | |
| | IASFRLFARTRSMWSFNPETNILLNVPLHGTILTRPLLESELVIGAVI | |
| | LRGHLRIAGHHLGRCDIKDLPKEITVATSRTLSYYKLGASQRVAGDSG | |
| | FAAYSRYRIGNYKLNTDHSSSSDNIALLVQ | |
| SARS-CoV-2 | MSDNGPQNQRNAPRITFGGPSDSTGSNQNGERSGARSKQRRPQGLPN | 62 |
| Nucleocapsid | NTASWFTALTQHGKEDLKFPRGQGVPINTNSSPDDQIGYYRRATRRI | |
| | RGGDGKMKDLSPRWYFYYLGTGPEAGLPYGANKDGIIWVATEGALNT | |
| | PKDHIGTRNPANNAAIVLQLPQGTTLPKGFYAEGSRGGSQASSRSSS | |
| | RSRNSSRNSTPGSSRGTSPARMAGNGGDAALALLLLDRLNQLESKMS | |
| | GKGQQQQGQTVTKKSAAEASKKPRQKRTATKAYNVTQAFGRRGPEQT | |
| | QGNFGDQELIRQGTDYKHWPQIAQFAPSASAFFGMSRIGMEVTPSGT | |
| | WLTYTGAIKLDDKDPNFKDQVILLNKHIDAYKTFPPTEPKKDKKKKA | |
| | DETQALPQRQKKQQTVTLLPAADLDDFSKQLQQSMSSADSTQA | |
| SARS-CoV-2 | MYSFVSEETGTLIVNSVLLFLAFVVFLLVTLAILTALRLCAYCCNIV | 63 |
| Envelope | NVSLVKPSFYVYSRVKNLNSSRVPDLLV | |
| SARS-CoV-2 | MFVFLVLLPLVSSQCVNLTTRTQLPPAYTNSFTRGVYYPDKVFRSS | 27974 |
| Spike | VLHSTQDLFLPFFSNVTWFHVISGTNGTKRFDNPVLPFNDGVYFAS | |
| (B.1.1.529 | IEKSNIIRGWIFGTTLDSKTQSLLIVNNATNVVIKVCEFQFCNDPF | |
| “Omicron” | LDHKNNKSWMESEFRVYSSANNCTFEYVSQPFLMDLEGKQGNFKNL | |
| isolate; | REFVFKNIDSGYFKIYSKHTPIIVREPEDLPQGFSALEPLVDLPIG | |
| modified); | INITRFQTLLALHRYLTPGDSSSGWTAGAAAYYVGYLQPRTFLLKY | |
| Includes | NENGTITDAVDCALDPLSETKCTLKSFTVEKGIYQTSNFRVQPTES | |
| mutations 679- | IVRFPNITNLCPFDEVENATRFASVYAWNRKRISNCVADYSVLYNL | |
| 682 RRAR [SEQ | APFFTFKCYGVSPTKLNDLCFTNVYADSFVIRGDEVRQIAPGQTGN | |
| ID NO: 125] to | IADYNYKLPDDFTGCVIAWNSNKLDSKVSGNYNYLYRLFRKSNLKP | |
| GSAS [SEQ ID | FERDISTEIYQAGNKPCNGVAGENCYFPLKSYSFRPTYGVGHQPYR | |
| NO: 126], K983P | VVVLSFELLHAPATVCGPKKSTNLVKNKCVNFNFNGLKGTGVLTES | |
| and V984P | NKKFLPFQQFGRDIADTTDAVRDPQTLEILDITPCSFGGVSVITPG | |
| | TNTSNQVAVLYQGVNCTEVPVAIHADQLTPTWRVYSTGSNVFQTRA | |
| | GCLIGAEYVNNSYECDIPIGAGICASYQTQTKSHGSASSVASQSII | |
| | AYTMSLGAENSVAYSNNSIAIPTNFTISVTTEILPVSMTKTSVDCT | |
| | MYICGDSTECSNLLLQYGSFCTQLKRALTGIAVEQDKNTQEVFAQV | |
| | KQIYKTPPIKYFGGFNFSQILPDPSKPSKRSFIEDLLFNKVTLADA | |
| | GFIKQYGDCLGDIAARDLICAQKFKGLTVLPPLLTDEMIAQYTSAL | |
| | LAGTITSGWTFGAGAALQIPFAMQMAYRENGIGVTQNVLYENQKLI | |
| | ANQFNSAIGKIQDSLSSTASALGKLQDVVNHNAQALNTLVKQLSSK | |
| | FGAISSVLNDIFSRLDPPEAEVQIDRLITGRLQSLQTYVTQQLIRA | |
| | AEIRASANLAATKMSECVLGQSKRVDFCGKGYHLMSFPQSAPHGVV | |
| | FLHVTYVPAQEKNFTTAPAICHDGKAHFPREGVFVSNGTHWFVTQR | |
| | NFYEPQIITTDNTFVSGNCDVVIGIVNNTVYDPLQPELDSFKEELD | |
| | KYFKNHTSPDVDLGDISGINASVVNIQKEIDRLNEVAKNLNESLID | |
| | LQELGKYEQYIKWPWYIWLGFIAGLIAIVMVTIMLCCMTSCCSCLK | |
| | GCCSCGSCCKFDEDDSEPVLKGVKLHYT | |
| SARS-CoV-2 | MFVFLVLLPLVSSQCVNLTTRTQLPPAYTNSFTRGVYYPDKVFRSS | 27977 |
| Spike | VLHSTQDLFLPFFSNVTWFHVISGTNGTKRFDNPVLPFNDGVYFAS | |
| (B.1.1.529 | IEKSNIIRGWIFGTTLDSKTQSLLIVNNATNVVIKVCEFQFCNDPF | |
| “Omicron” | LDHKNNKSWMESEFRVYSSANNCTFEYVSQPFLMDLEGKQGNFKNL | |
| isolate native) | REFVFKNIDGYFKIYSKHTPIIVREPEDLPQGFSALEPLVDLPIGI | |
| | NITRFQTLLALHRSYLTPGDSSSGWTAGAAAYYVGYLQPRTFLLKY | |
| | NENGTITDAVDCALDPLSETKCTLKSFTVEKGIYQTSNFRVQPTES | |
| | IVRFPNITNLCPFDEVENATRFASVYAWNRKRISNCVADYSVLYNL | |
| | APFFTFKCYGVSPTKLNDLCFTNVYADSFVIRGDEVRQIAPGQTGN | |
| | IADYNYKLPDDFTGCVIAWNSNKLDSKVSGNYNYLYRLFRKSNLKP | |
| | FERDISTEIYQAGNKPCNGVAGENCYFPLKSYSFRPTYGVGHQPYR | |
| | VVVLSFELLHAPATVCGPKKSTNLVKNKCVNFNFNGLKGTGVLTES | |
| | NKKFLPFQQFGRDIADTTDAVRDPQTLEILDITPCSFGGVSVITPG | |
| | TNTSNQVAVLYQGVNCTEVPVAIHADQLTPTWRVYSTGSNVFQTRA | |
| | GCLIGAEYVNNSYECDIPIGAGICASYQTQTKSHRRARSVASQSII | |
| | AYTMSLGAENSVAYSNNSIAIPTNFTISVTTEILPVSMTKTSVDCT | |
| | MYICGDSTECSNLLLQYGSFCTQLKRALTGIAVEQDKNTQEVFAQV | |
| | KQIYKTPPIKYFGGFNFSQILPDPSKPSKRSFIEDLLFNKVTLADA | |
| | GFIKQYGDCLGDIAARDLICAQKFKGLTVLPPLLTDEMIAQYTSAL | |
| | LAGTITSGWTFGAGAALQIPFAMQMAYRENGIGVTQNVLYENQKLI | |
| | ANQFNSAIGKIQDSLSSTASALGKLQDVVNHNAQALNTLVKQLSSK | |
| | FGAISSVLNDIFSRLDKVEAEVQIDRLITGRLQSLQTYVTQQLIRA | |
| | AEIRASANLAATKMSECVLGQSKRVDFCGKGYHLMSFPQSAPHGVV | |
| | FLHVTYVPAQEKNFTTAPAICHDGKAHFPREGVFVSNGTHWFVTQR | |
| | NFYEPQIITTDNTFVSGNCDVVIGIVNNTVYDPLQPELDSFKEELD | |
| | KYFKNHTSPDVDLGDISGINASVVNIQKEIDRLNEVAKNLNESLID | |
| | LQELGKYEQYIKWPWYIWLGFIAGLIAIVMVTIMLCCMTSCCSCLK | |
| | GCCSCGSCCKFDEDDSEPVLKGVKLHYT | |
| Concatenated | MAGNVAFQTVKPGNFNKDFYDFAVSKGFFKEGSSGASQRVAGDSGF | |
| EDGE predicted | AAYSRYRIGNDGVPFVVSTGYHFRELGVYLTFYLTNDVSFLAHIQW | 27975 |
| SARS-CoV-2 | MVMFTPGLMWLSYFIASFRLFARTRSMFEYYHTTDPSFLGRYMSAL | |
| MHC Class I | NHTKKWKYPQISNEKQEILGTVSWNLREATTRQVVNVVTTKIALKT | |
| Epitope Cassette | LACFVLAAVYRINWITGPGPGAKFVAAWTLKAAAGPGPGQYIKANS | |
| “TCE11” with | KFIGITELGPGPG | |
| N-term leader | | |
| (bold) and C- | | |
| term Universal | | |
| MHC Class II | | |
| with GPGPG | | |
| linkers (SEQ ID | | |
| NO: 56) (bold | | |
| italic) | | |
| SARS-CoV-2 | MFVFLVLLPLVSSQCVNLITRTQSYTNSFTRGVYYPDKVFRSSVLHST | 27978 |
| Spike | QDLFLPFFSNVTWFHAISGTNGTKRFDNPVLPFNDGVYFASTEKSNIIR | |
| (B.1.1.529 | GWIFGTTLDSKTQSLLIVNNATNVVIKVCEFQFCNDPFLDVYYHKNNKS | |
| “Omicron” BA5 | WMESEFRVYSSANNCTFEYVSQPFLMDLEGKQGNFKNLREFVFKNIDG | |
| subvariant; | YFKIYSKHTPINLGRDLPQGFSALEPLVDLPIGINITRFQTLLALHRS | |
| modified); | YLTPGDSSSGWTAGAAAYYVGYLQPRTFLLKYNENGTITDAVDCALDP | |
| Includes | LSETKCTLKSFTVEKGIYQTSNFRVQPTESIVRFPNITNLCPFDEVEN | |
| mutations 677- | ATRFASVYAWNRKRISNCVADYSVLYNFAPFFAFKCYGVSPTKLNDL | |
| 680 RRAR [SEQ | CFTNVYADSFVIRGNEVSQIAPGQTGNIADYNYKLPDDFTGCVIAWNS | |
| ID NO: 125] to | NKLDSKVGGNYNYRYRLFRKSNLKPFERDISTEIYQAGNKPCNGVAG | |
| GSAS [SEQ ID | VNCYFPLQSYGFRPTYGVGHQPYRVVVLSFELLHAPATVCGPKKSTN | |
| NO: 126], K981P | LVKNKCVNFNFNGLTGTGVLTESNKKFLPFQQFGRDIADTTDAVRDP | |
| and V982P | QTLEILDITPCSFGGVSVITPGTNTSNQVAVLYQGVNCTEVPVAIHA | |
| | DQLTPTWRVYSTGSNVFQTRAGCLIGAEYVNNSYECDIPIGAGICAS | |
| | YQTQTKSHGSASSVASQSIIAYTMSLGAENSVAYSNNSIAIPTNFTI | |
| | SVTTEILPVSMTKTSVDCTMYICGDSTECSNLLLQYGSFCTQLKRAL | |
| | TGIAVEQDKNTQEVFAQVKQIYKTPPIKYFGGFNFSQILPDPSKPSK | |
| | RSFIEDLLENKVTLADAGFIKQYGDCLGDIAARDLICAQKENGLTVL | |
| | PPLLTDEMIAQYTSALLAGTITSGWTFGAGAALQIPFAMQMAYRENG | |
| | IGVTQNVLYENQKLIANQFNSAIGKIQDSLSSTASALGKLQDVVNHN | |
| | AQALNTLVKQLSSKFGAISSVLNDILSRLDPPEAEVQIDRLITGRLQ | |
| | SLQTYVTQQLIRAAEIRASANLAATKMSECVLGQSKRVDFCGKGYHL | |
| | MSFPQSAPHGVVFLHVTYVPAQEKNFTTAPAICHDGKAHFPREGVFV | |
| | SNGTHWFVTQRNFYEPQIITTDNTFVSGNCDVVIGIVNNTVYDPLQP | |
| | ELDSFKEELDKYFKNHTSPDVDLGDISGINASVVNIQKEIDRLNEVA | |
| | IKNLNESLIDLQELGKYEQYKWPWYIWLGFIAGLIAIVMVTIMLCCM | |
| | TSCCSCLKGCCSCGSCCKFDEDDSEPVLKGVKLHYT | |
| SARS-CoV-2 | MFVFLVLLPLVSSQCVNLITRTQSYTNSFTRGVYYPDKVFRSSVLH | 27979 |
| Spike | STQDLFLPFFSNVTWFHAISGTNGTKRFDNPVLPFNDGVYFASTEK | |
| (BA5 “Omicron” | SNIIRGWIFGTTLDSKTQSLLIVNNATNVVIKVCEFQFCNDPFLDV | |
| variant native) | YYHKNNKSWMESEFRVYSSANNCTFEYVSQPFLMDLEGKQGNFKNL | |
| | REFVFKNIDGYFKIYSKHTPINLGRDLPQGFSALEPLVDLPIGINI | |
| | TRFQTLLALHRSYLTPGDSSSGWTAGAAAYYVGYLQPRTFLLKYNE | |
| | NGTITDAVDCALDPLSETKCTLKSFTVEKGIYQTSNFRVQPTESIV | |
| | RFPNITNLCPFDEVENATRFASVYAWNRKRISNCVADYSVLYNFAP | |
| | FFAFKCYGVSPTKLNDLCFTNVYADSFVIRGNEVSQIAPGQTGNIA | |
| | DYNYKLPDDFTGCVIAWNSNKLDSKVGGNYNYRYRLFRKSNLKPFE | |
| | RDISTEIYQAGNKPCNGVAGVNCYFPLQSYGFRPTYGVGHQPYRVV | |
| | VLSFELLHAPATVCGPKKSTNLVKNKCVNFNFNGLTGTGVLTESNK | |
| | KFLPFQQFGRDIADTTDAVRDPQTLEILDITPCSFGGVSVITPGTN | |
| | TSNQVAVLYQGVNCTEVPVAIHADQLTPTWRVYSTGSNVFQTRAGC | |
| | LIGAEYVNNSYECDIPIGAGICASYQTQTKSHRRARSVASQSIIAY | |
| | TMSLGAENSVAYSNNSIAIPTNFTISVTTEILPVSMTKTSVDCTMY | |
| | ICGDSTECSNLLLQYGSFCTQLKRALTGIAVEQDKNTQEVFAQVKQ | |
| | IYKTPPIKYFGGFNFSQILPDPSKPSKRSFIEDLLENKVTLADAGF | |
| | IKQYGDCLGDIAARDLICAQKFNGLTVLPPLLTDEMIAQYTSALLA | |
| | GTITSGWTFGAGAALQIPFAMQMAYRENGIGVTQNVLYENQKLIAN | |
| | QFNSAIGKIQDSLSSTASALGKLQDVVNHNAQALNTLVKQLSSKFG | |
| | AISSVLNDILSRLDKVEAEVQIDRLITGRLQSLQTYVTQQLIRAAE | |
| | IRASANLAATKMSECVLGQSKRVDFCGKGYHLMSFPQSAPHGVVFL | |
| | HVTYVPAQEKNFTTAPAICHDGKAHFPREGVFVSNGTHWFVTQRNF | |
| | YEPQIITTDNTFVSGNCDVVIGIVNNTVYDPLQPELDSFKEELDKY | |
| | FKNHTSPDVDLGDISGINASVVNIQKEIDRLNEVAKNLNESLIDLQ | |
| | ELGKYEQYIKWPWYIWLGFIAGLIAIVMVTIMLCCMTSCCSCLKGC | |
| | CSCGSCCKFDEDDSEPVLKGVKLHYT | |

TABLE 4
SARS-CoV-2 Vaccine Designs

| Vector Insert | SEQ ID NO: |
|---|---|
| EDGE Predicted Epitopes (EPE): Concatenated EDGE predicted SARS- | 64 |
| CoV-2 MHC Class I Epitope Cassette with 5′ expression leader (bold) and 3′ | |
| Universal MHC Class II with GPGPG linkers (SEQ ID NO: 56) (bold italic) | |
| CMV-EPE-BGH | 65 |
| CMV-Spike (IDT)-T2A-membrane protein-SV40 | 66 |
| CMV-Spike (IDT)-T2A-membrane protein-BGH | 67 |
| CMV-Spike (IDT)-SV40-CMV-EPE-BGH | 68 |
| CMV-Spike (IDT)-SV40 | 69 |
| CMV-Modified Spike (IDT)-SV40 | 70 |
| CMV-Spike (IDT)-T2A-Envelope-SV40-CMV-nucleocapsid-T2A-membrane | 71 |
| protein-SV40 | |
| CMV-Spike (IDT)-SV40 -CMV-nucleocapsid-T2A-membrane protein-BGH | 72 |
| CMV-Spike (IDT)-T2A-envelope protein-T2A-membrane protein-SV40 | 73 |
| CMV-Spike (IDT)-T2A-envelope protein-T2A-membrane protein-SV40- | 74 |
| CMV-EPE-BGH | |
| SAM-Nuc-TCE11-SpikeB.1.1.529 sequence [GRT-R918] | 27976 |
| SAM-Nuc-TCE11-SpikeB.1.1.529 BA5 subisolate sequence [GRT-R920] | 27976 with Spike |
| | encoding sequences |
| | substituted with 27980 |
| SAM-N-TCE11-Spike beta (501Y.V2) [GRT-R912] | 27981 |
| SAM-TCE9-Spike beta (501Y.V2) [GRT-R914] | 27982 |
| SAM-SGP1-TCE5-SGP2-CTSpikeGF2P | 27983 |

A RNA alphavirus backbone for the antigen expression system was generated from a self-replicating Venezuelan Equine Encephalitis (VEE) virus (Kinney, 1986, Virology 152: 400-413) by deleting the structural proteins of VEE located 3′ of the 26S sub-genomic promoter (VEE sequences 7544 to 11,175 deleted; numbering based on Kinney et al 1986; SEQ ID NO:6). To generate the self-amplifying mRNA (“SAM”) vector, the deleted sequences are replaced by antigen sequences. A representative SAM vector containing 20 model antigens is “VEE-MAG25mer” (SEQ ID NO:4). The vectors featuring the antigen cassettes described having the MAG25mer cassette can be replaced by the SARS-CoV-2 cassettes and/or full-length proteins described herein.

### In Vitro Transcription to Generate SAM

For in vivo studies: SAM vectors were generated as “AU-SAM” vectors. A modified T7 RNA polymerase promoter (TAATACGACTCACTATA; SEQ ID NO: 120), which lacks the canonical 3′ dinucleotide GG, was added to the 5′ end of the SAM vector to generate the in vitro transcription template DNA (SEQ ID NO:77; 7544 to 11,175 deleted without an inserted antigen cassette). Reaction conditions are described below:
1× transcription buffer (40 mM Tris-HCL [pH7.9], 10 mM dithiothreitol, 2 mM spermidine, 0.002% Triton X-100, and 27 mM magnesium chloride) using final concentrations of 1×T7 RNA polymerase mix (E2040S); 0.025 mg/mL DNA transcription template (linearized by restriction digest); 8 mM CleanCap Reagent AU (Cat. No. N-7114) and 10 mM each of ATP, cytidine triphosphate (CTP), GTP, and uridine triphosphate (UTP)
Transcription reactions were incubated at 37° C. for 2 hr and treated with final 2 U DNase I (AM2239)/0.001 mg DNA transcription template in DNase I buffer for 1 hr at 37° C.
SAM was purified by RNeasy Maxi (QIAGEN, 75162)

Alternatively to co-transcriptional addition of a 5′ cap structure, a 7-methylguanosine or a related 5′ cap structure can be enzymatically added following transcription using a vaccinia capping system (NEB Cat. No. M2080) containing mRNA 2′-O-methyltransferase and S-Adenosyl methionine.

### Adenoviral Vectors

A modified ChAdV68 vector (“chAd68-Empty-E4deleted” SEQ ID NO:75) for the antigen expression system was generated based on AC_000011.1 with E1 (nt 577 to 3403), E3 (nt 27,125-31,825), and E4 region (nt 34,916 to 35,642) sequences deleted and the corresponding ATCC VR-594 (Independently sequenced Full-Length VR-594 C68 SEQ ID NO:10) nucleotides substituted at five positions. The full-length ChAdV68 AC_000011.1 sequence with corresponding ATCC VR-594 nucleotides substituted at five positions is referred to as “ChAdV68.5WTnt” (SEQ ID NO:1). Antigen cassettes under the control of the CMV promoter/enhancer are inserted in place of deleted E1 sequences.

### Adenoviral Production in 293F Cells

ChAdV68 virus production are performed in 293F cells grown in 293 FreeStyle™ (ThermoFisher) media in an incubator at 8% CO₂. On the day of infection cells are diluted to 10⁶ cells per mL, with 98% viability and 400 mL are used per production run in 1 L Shake flasks (Corning). 4 mL of the tertiary viral stock with a target MOI of >3.3 is used per infection. The cells are incubated for 48-72 h until the viability was <70% as measured by Trypan blue. The infected cells are then harvested by centrifugation, full speed bench top centrifuge and washed in 1×PBS, re-centrifuged and then re-suspended in 20 mL of 10 mM Tris pH7.4. The cell pellet is lysed by freeze thawing 3× and clarified by centrifugation at 4,300×g for 5 minutes.

### Adenoviral Purification by CsCl Centrifugation

Viral DNA is purified by CsCl centrifugation. Two discontinuous gradient runs are performed. The first to purify virus from cellular components and the second to further refine separation from cellular components and separate defective from infectious particles.

10 mL of 1.2 (26.8 g CsCl dissolved in 92 mL of 10 mM Tris pH 8.0) CsCl is added to polyallomer tubes. Then 8 mL of 1.4 CsCl (53 g CsCl dissolved in 87 mL of 10 mM Tris pH 8.0) is carefully added using a pipette delivering to the bottom of the tube. The clarified virus is carefully layered on top of the 1.2 layer. If needed more 10 mM Tris is added to balance the tubes. The tubes are then placed in a SW-32Ti rotor and centrifuged for 2 h 30 min at 10° C. The tube are then removed to a laminar flow cabinet and the virus band pulled using an 18 gauge needle and a 10 mL syringe. Care is taken not to remove contaminating host cell DNA and protein. The band is then diluted at least 2× with 10 mM Tris pH 8.0 and layered as before on a discontinuous gradient as described above. The run is performed as described before except that this time the run is performed overnight. The next day the band is pulled with care to avoid pulling any of the defective particle band. The virus is then dialyzed using a Slide-a-Lyzer™ Cassette (Pierce) against ARM buffer (20 mM Tris pH 8.0, 25 mM NaCl, 2.5% Glycerol). This is performed 3×, 1 h per buffer exchange. The virus is then aliquoted for storage at −80° C.

### Adenoviral Viral Assays

VP concentration is performed by using an OD 260 assay based on the extinction coefficient of 1.1×10¹² viral particles (VP) is equivalent to an Absorbance value of 1 at OD260 nm. Two dilutions (1:5 and 1:10) of adenovirus are made in a viral lysis buffer (0.1% SDS, 10 mM Tris pH 7.4, 1 mM EDTA). OD is measured in duplicate at both dilutions and the VP concentration/mL is measured by multiplying the OD260 value X dilution factor X 1.1×10¹²VP.

An infectious unit (IU) titer is calculated by a limiting dilution assay of the viral stock. The virus is initially diluted 100× in DMEM/5% NS/1×PS and then subsequently diluted using 10-fold dilutions down to 1×10⁻⁷. 100 μL of these dilutions are then added to 293A cells that were seeded at least an hour before at 3e5 cells/well of a 24 well plate. This is performed in duplicate. Plates are incubated for 48 h in a CO2 (5%) incubator at 37° C. The cells are then washed with 1×PBS and are then fixed with 100% cold methanol (−20° C.). The plates are then incubated at −20° C. for a minimum of 20 minutes. The wells are washed with 1×PBS then blocked in 1×PBS/0.1% BSA for 1 h at room temperature. A rabbit anti-Ad antibody (Abcam, Cambridge, MA) is added at 1:8,000 dilution in blocking buffer (0.25 ml per well) and incubated for 1 h at room temperature. The wells are washed 4× with 0.5 mL PBS per well. A HRP conjugated Goat anti-Rabbit antibody (Bethyl Labs, Montgomery Texas) diluted 1000× is added per well and incubated for 1 h prior to a final round of washing. 5 PBS washes are performed and the plates were developed using DAB (Diaminobenzidine tetrahydrochloride) substrate in Tris buffered saline (0.67 mg/mL DAB in 50 mM Tris pH 7.5, 150 mM NaCl) with 0.01% H₂O₂. Wells are developed for 5 min prior to counting. Cells are counted under a 10× objective using a dilution that gave between 4-40 stained cells per field of view. The field of view that is used was a 0.32 mm² grid of which there are equivalent to 625 per field of view on a 24 well plate. The number of infectious viruses/mL can be determined by the number of stained cells per grid multiplied by the number of grids per field of view multiplied by a dilution factor 10. Similarly, when working with GFP expressing cells florescent can be used rather than capsid staining to determine the number of GFP expressing virions per mL.

### XIV.C. Spike Protein Sequence Optimization

Various sequence-optimized nucleotide sequences encoding the Spike protein were evaluated in ChAdV68 vaccine vectors.

### Sequence-Optimization of Spike Sequence

The Spike nucleotide sequence from Wuhan Hu/1 (SEQ ID NO:78) was sequence-optimized by substituting synonymous codons such that the amino acid sequence was unaffected. An IDT algorithm was used for enhanced expression in humans and for reduced complexity to aid synthesis (see, e.g., SEQ ID NOs:66-74). The Spike sequence was additionally sequence-optimized using two additional algorithms; (1) a single sequence (SEQ ID NO:87) generated using SGI DNA (La Jolla, CA); (2) 6 sequences designated CT1, CT20, CT56, CT83, CT131, and CT 199 (SEQ ID NOs:79-84) generated using COOL (COOL algorithm generates multiple sequences and 6 were selected). The sequences of each are presented in Table 5.

Splicing events were identified in cDNA from 293A cells infected with ChAdV68 viruses or transfected with ChAdV68 genomic DNA. Specifically, total RNA from 10ₑ5-10ₑ6 cells was purified using Qiagen's RNeasy columns. Residual DNA was removed by DNAse treatment, and cDNA was produced using SuperScriptlV reverse transcriptase (Thermo). Subsequently, primers specific for the 5′ UTR and 3′ UTR of the Gritstone ChAdV68 cassette were used to generate PCR products, analyzed by agarose gel electrophoresis, gel-purified, and Sanger-sequenced to identify regions deleted by splicing.

Splice donor sites were removed by site-directed mutagenesis disrupting the nucleotide sequence motif while not disturbing the amino acid sequence. Mutagenesis was accomplished by incorporating above mutations into PCR primers, amplifying several fragments in parallel, and running a Gibson assembly on the fragments (overlapping by 30-60 nt). Optimized clone CT1-2C (SEQ ID NO:85) had Sanger sequence-identified splice donor motifs at NT385 and NT539 mutated, and clone IDT-4C (SEQ ID NO:86) had Sanger sequence-identified splice donor motifs at NT385, NT539, and predicted donor motifs at NT2003, and NT2473 mutated. Additionally, a possible polyadenylation site AATAAA at nt 445 was mutated to AAcAAA in IDT-4C clone.

The sequences described are presented in Table 5. SAM-Nuc-TCE11-SpikeB.1.1.529 (SEQ ID NO: 27976) includes a SARS-CoV-2 Spike protein encoded by Cool Tool optimized sequence version 1 (SEQ ID NO:79) with 37 Omicron mutations manually changed within the sequence, as applicable (see SEQ ID NO: 27977).

TABLE 5
Sequence-optimized Spike Sequences

| | | SEQ ID |
|---|---|---|
| Spike Sequence | Nucleic Acid Sequence | NO: |
| Spike Native; | atgtttgtttttcttgttttattgccactagtctctagtcagtgt | 78 |
| NC_045512.2 | gttaatcttacaaccagaactcaattaccccctgcatacactaat | |
| Severe acute | tctttcacacgtggtgtttattaccctgacaaagttttcagatcc | |
| respiratory | tcagttttacattcaactcaggacttgttcttacctttcttttcc | |
| syndrome | aatgttacttggttccatgctatacatgtctctgggaccaatggt | |
| coronavirus 2 | actaagaggtttgataaccctgtcctaccatttaatgatggtgtt | |
| isolate Wuhan- | tattttgcttccactgagaagtctaacataataagaggctggatt | |
| Hu-1 | tttggtactactttagattcgaagacccagtccctacttattgtt | |
| | aataacgctactaatgttgttattaaagtctgtgaatttcaattt | |
| | tgtaatgatccatttttgggtgtttattaccacaaaaacaacaaa | |
| | agttggatggaaagtgagttcagagtttattctagtgcgaataat | |
| | tgcacttttgaatatgtctctcagccttttcttatggaccttgaa | |
| | ggaaaacagggtaatttcaaaaatcttagggaatttgtgtttaag | |
| | aatattgatggttattttaaaatatattctaagcacacgcctatt | |
| | aatttagtgcgtgatctccctcagggtttttcggctttagaacca | |
| | ttggtagatttgccaataggtattaacatcactaggtttcaaact | |
| | ttacttgctttacatagaagttatttgactcctggtgattcttct | |
| | tcaggttggacagctggtgctgcagcttattatgtgggttatctt | |
| | caacctaggacttttctattaaaatataatgaaaatggaaccatt | |
| | acagatgctgtagactgtgcacttgaccctctctcagaaacaaag | |
| | tgtacgttgaaatccttcactgtagaaaaaggaatctatcaaact | |
| | tctaactttagagtccaaccaacagaatctattgttagatttcct | |
| | aatattacaaacttgtgcccttttggtgaagtttttaacgccacc | |
| | agatttgcatctgtttatgcttggaacaggaagagaatcagcaac | |
| | tgtgttgctgattattctgtcctatataattccgcatcattttcc | |
| | acttttaagtgttatggagtgtctcctactaaattaaatgatctc | |
| | tgctttactaatgtctatgcagattcatttgtaattagaggtgat | |
| | gaagtcagacaaatcgctccagggcaaactggaaagattgctgat | |
| | tataattataaattaccagatgattttacaggctgcgttatagct | |
| | tggaattctaacaatcttgattctaaggttggtggtaattataat | |
| | tacctgtatagattgtttaggaagtctaatctcaaaccttttgag | |
| | agagatatttcaactgaaatctatcaggccggtagcacaccttgt | |
| | aatggtgttgaaggttttaattgttactttcctttacaatcatat | |
| | ggtttccaacccactaatggtgttggttaccaaccatacagagta | |
| | gtagtactttcttttgaacttctacatgcaccagcaactgtttgt | |
| | ggacctaaaaagtctactaatttggttaaaaacaaatgtgtcaat | |
| | ttcaacttcaatggtttaacaggcacaggtgttcttactgagtct | |
| | aacaaaaagtttctgcctttccaacaatttggcagagacattgct | |
| | gacactactgatgctgtccgtgatccacagacacttgagattctt | |
| | gacattacaccatgttcttttggtggtgtcagtgttataacacca | |
| | ggaacaaatacttctaaccaggttgctgttctttatcaggatgtt | |
| | aactgcacagaagtccctgttgctattcatgcagatcaacttact | |
| | cctacttggcgtgtttattctacaggttctaatgtttttcaaaca | |
| | cgtgcaggctgtttaataggggctgaacatgtcaacaactcatat | |
| | gagtgtgacatacccattggtgcaggtatatgcgctagttatcag | |
| | actcagactaattctcctcggcgggcacgtagtgtagctagtcaa | |
| | tccatcattgcctacactatgtcacttggtgcagaaaattcagtt | |
| | gcttactctaataactctattgccatacccacaaattttactatt | |
| | agtgttaccacagaaattctaccagtgtctatgaccaagacatca | |
| | gtagattgtacaatgtacatttgtggtgattcaactgaatgcagc | |
| | aatcttttgttgcaatatggcagtttttgtacacaattaaaccgt | |
| | gctttaactggaatagctgttgaacaagacaaaaacacccaagaa | |
| | gtttttgcacaagtcaaacaaatttacaaaacaccaccaattaaa | |
| | gattttggtggttttaatttttcacaaatattaccagatccatca | |
| | aaaccaagcaagaggtcatttattgaagatctacttttcaacaaa | |
| | gtgacacttgcagatgctggcttcatcaaacaatatggtgattgc | |
| | cttggtgatattgctgctagagacctcatttgtgcacaaaagttt | |
| | aacggccttactgttttgccacctttgctcacagatgaaatgatt | |
| | gctcaatacacttctgcactgttagcgggtacaatcacttctggt | |
| | tggacctttggtgcaggtgctgcattacaaataccatttgctatg | |
| | caaatggcttataggtttaatggtattggagttacacagaatgtt | |
| | ctctatgagaaccaaaaattgattgccaaccaatttaatagtgct | |
| | attggcaaaattcaagactcactttcttccacagcaagtgcactt | |
| | ggaaaacttcaagatgtggtcaaccaaaatgcacaagctttaaac | |
| | acgcttgttaaacaacttagctccaattttggtgcaatttcaagt | |
| | gttttaaatgatatcctttcacgtcttgacaaagttgaggctgaa | |
| | gtgcaaattgataggttgatcacaggcagacttcaaagtttgcag | |
| | acatatgtgactcaacaattaattagagctgcagaaatcagagct | |
| | tctgctaatcttgctgctactaaaatgtcagagtgtgtacttgga | |
| | caatcaaaaagagttgatttttgtggaaagggctatcatcttatg | |
| | tccttccctcagtcagcacctcatggtgtagtcttcttgcatgtg | |
| | acttatgtccctgcacaagaaaagaacttcacaactgctcctgcc | |
| | atttgtcatgatggaaaagcacactttcctcgtgaaggtgtcttt | |
| | gtttcaaatggcacacactggtttgtaacacaaaggaatttttat | |
| | gaaccacaaatcattactacagacaacacatttgtgtctggtaac | |
| | tgtgatgttgtaataggaattgtcaacaacacagtttatgatcct | |
| | ttgcaacctgaattagactcattcaaggaggagttagataaatat | |
| | tttaagaatcatacatcaccagatgttgatttaggtgacatctct | |
| | ggcattaatgcttcagttgtaaacattcaaaaagaaattgaccgc | |
| | ctcaatgaggttgccaagaatttaaatgaatctctcatcgatctc | |
| | caagaacttggaaagtatgagcagtatataaaatggccatggtac | |
| | atttggctaggttttatagctggcttgattgccatagtaatggtg | |
| | acaattatgctttgctgtatgaccagttgctgtagttgtctcaag | |
| | ggctgttgttcttgtggatcctgctgcaaatttgatgaagacgac | |
| | tctgagccagtgctcaaaggagtcaaattacattacacataa | |
| Spike CT1 | ATGTTTGTCTTCCTGGTCTTGCTGCCGCTGGTGAGCAGCCAGTGC | 79 |
| Optimized | GTGAATCTCACCACCCGCACCCAGCTTCCACCTGCCTACACTAAC | |
| | AGCTTCACCCGAGGGGTGTATTACCCTGACAAGGTATTCCGGTCC | |
| | TCCGTCCTCCATAGCACGCAGGACCTTTTTCTGCCCTTCTTCTCA | |
| | AATGTGACATGGTTCCATGCCATTCACGTGAGCGGCACGAATGGA | |
| | ACGAAGCGCTTTGATAACCCCGTGCTGCCTTTCAATGACGGCGTC | |
| | TACTTCGCCTCCACTGAAAAGTCAAACATCATCCGGGGCTGGATC | |
| | TTTGGCACCACTCTTGATTCAAAGACCCAGTCACTGCTGATTGTG | |
| | AACAATGCTACAAACGTGGTTATCAAGGTGTGTGAGTTTCAGTTC | |
| | TGTAACGATCCATTTTTGGGAGTGTACTACCACAAGAACAACAAG | |
| | TCCTGGATGGAGTCTGAGTTCAGAGTGTATAGCTCTGCTAACAAC | |
| | TGCACCTTCGAGTACGTGTCCCAGCCTTTCCTTATGGACCTGGAA | |
| | GGCAAACAGGGCAATTTCAAAAACCTGAGAGAGTTCGTGTTTAAG | |
| | AACATTGACGGATACTTCAAAATTTATTCTAAGCACACACCAATT | |
| | AACTTAGTGCGGGACCTACCCCAAGGCTTTAGCGCCCTAGAGCCC | |
| | CTGGTTGACCTGCCCATTGGGATCAATATAACAAGGTTCCAAACT | |
| | CTACTGGCTCTGCATAGAAGTTATCTGACCCCAGGAGACAGCTCT | |
| | AGTGGTTGGACCGCCGGCGCAGCAGCCTACTATGTCGGGTACTTA | |
| | CAGCCACGCACGTTCCTTCTGAAGTACAATGAGAACGGGACAATC | |
| | ACTGACGCAGTAGACTGTGCACTGGACCCGCTAAGCGAGACTAAG | |
| | TGCACACTTAAATCCTTCACGGTGGAGAAAGGCATTTATCAGACC | |
| | TCTAACTTCAGGGTGCAGCCAACAGAAAGCATTGTGCGATTCCCA | |
| | AATATTACTAATCTTTGCCCTTTCGGGGAGGTCTTTAATGCAACT | |
| | AGATTCGCATCAGTCTATGCGTGGAACCGCAAACGCATTTCCAAT | |
| | TGTGTCGCAGACTACTCAGTGCTGTACAACTCTGCCTCTTTCAGT | |
| | ACGTTCAAGTGTTACGGAGTGTCACCCACTAAACTGAACGACCTG | |
| | TGCTTTACAAATGTCTACGCTGACTCCTTCGTGATTAGGGGAGAC | |
| | GAGGTGAGACAAATTGCCCCCGGACAGACTGGGAAGATTGCCGAC | |
| | TACAATTATAAGCTTCCTGATGATTTCACTGGCTGTGTTATTGCC | |
| | TGGAATAGTAACAATCTGGATAGCAAGGTGGGAGGCAACTATAAC | |
| | TACTTATATCGACTGTTTAGGAAGAGTAATCTGAAACCATTTGAG | |
| | CGGGATATTTCCACAGAAATTTACCAGGCCGGGAGCACACCATGT | |
| | AATGGGGTGGAGGGATTTAATTGTTACTTCCCACTCCAGAGCTAT | |
| | GGTTTCCAACCCACCAATGGAGTGGGTTACCAGCCCTATAGAGTC | |
| | GTGGTGCTTAGTTTTGAGCTGCTTCACGCCCCAGCAACCGTCTGC | |
| | GGTCCCAAAAAGTCGACCAATCTCGTGAAAAACAAATGCGTAAAC | |
| | TTCAACTTTAACGGCTTAACAGGAACCGGCGTGCTCACCGAAAGC | |
| | AACAAGAAATTCCTTCCATTTCAGCAATTCGGAAGGGACATCGCC | |
| | GACACAACAGACGCGGTGAGGGACCCACAGACTCTGGAGATACTG | |
| | GACATCACTCCTTGTTCGTTTGGGGGCGTCTCGGTCATCACACCC | |
| | GGGACTAATACTAGTAATCAGGTAGCAGTTTTATATCAAGGCGTC | |
| | AACTGTACCGAAGTACCTGTGGCCATACACGCTGATCAGCTAACG | |
| | CCAACATGGCGAGTCTATTCCACCGGCTCTAACGTTTTTCAGACC | |
| | AGGGCTGGGTGCCTGATAGGGGCAGAGCACGTCAATAATTCCTAT | |
| | GAGTGTGATATCCCCATAGGTGCGGGGATCTGTGCCAGCTATCAA | |
| | ACCCAAACCAATTCACCAAGGCGAGCACGGTCTGTGGCTTCTCAG | |
| | AGCATAATTGCATATACAATGTCACTGGGCGCTGAGAATAGCGTT | |
| | GCATACTCTAATAACAGCATAGCCATTCCCACGAACTTTACTATC | |
| | AGTGTGACAACCGAAATATTGCCAGTTTCGATGACCAAAACTAGC | |
| | GTGGATTGCACGATGTACATCTGTGGAGACTCTACCGAATGCAGC | |
| | AATCTGCTATTACAATATGGCAGCTTCTGTACACAGTTAAATCGA | |
| | GCCTTGACAGGCATCGCAGTGGAACAGGACAAAAATACTCAAGAG | |
| | GTGTTTGCACAGGTGAAGCAAATCTACAAAACGCCCCCCATTAAA | |
| | GATTTTGGCGGGTTCAATTTTTCACAAATTCTCCCCGACCCGTCT | |
| | AAGCCGAGTAAGCGGTCCTTCATCGAAGATCTGCTCTTTAACAAA | |
| | GTAACCCTCGCCGATGCCGGCTTTATTAAGCAGTATGGCGACTGC | |
| | CTGGGGGATATAGCCGCTCGTGACCTGATTTGCGCCCAGAAGTTC | |
| | AATGGTCTGACCGTGTTGCCTCCTTTATTGACCGATGAAATGATT | |
| | GCCCAGTACACTAGTGCCCTGCTGGCCGGCACTATCACGTCTGGG | |
| | TGGACCTTCGGAGCTGGTGCCGCCTTGCAGATACCTTTTGCAATG | |
| | CAGATGGCCTATAGGTTTAATGGTATCGGAGTGACTCAGAACGTA | |
| | CTGTACGAGAACCAGAAGCTCATCGCTAATCAATTTAACTCCGCT | |
| | ATCGGAAAAATCCAGGACAGCCTCTCTTCTACAGCTAGCGCTCTG | |
| | GGCAAACTGCAGGATGTCGTTAATCAGAATGCCCAGGCCCTGAAC | |
| | ACCTTGGTTAAACAACTATCTTCCAACTTCGGGGCCATATCCAGT | |
| | GTGTTGAATGATATTCTCTCCCGCTTGGATAAGGTGGAAGCTGAG | |
| | GTGCAGATCGATCGCTTGATCACCGGCAGACTGCAGTCCCTCCAG | |
| | ACATATGTAACTCAGCAGCTGATTAGAGCCGCCGAGATAAGGGCA | |
| | AGTGCGAATCTGGCTGCCACCAAGATGAGCGAATGTGTATTGGGC | |
| | CAGAGCAAACGAGTTGATTTTTGCGGTAAGGGGTATCATTTAATG | |
| | TCTTTCCCTCAATCCGCACCTCATGGCGTAGTTTTCCTGCATGTG | |
| | ACTTATGTCCCGGCTCAGGAGAAGAATTTTACCACAGCCCCCGCG | |
| | ATCTGCCATGACGGAAAGGCCCACTTCCCCCGGGAAGGCGTGTTT | |
| | GTATCCAATGGGACTCACTGGTTTGTCACTCAGCGAAATTTTTAT | |
| | GAACCACAGATCATCACCACTGACAACACATTTGTTAGTGGAAAC | |
| | TGCGATGTGGTCATCGGCATCGTGAATAACACTGTCTATGATCCA | |
| | CTGCAACCTGAACTGGATTCTTTTAAAGAGGAACTCGACAAGTAT | |
| | TTTAAAAACCACACTAGCCCTGACGTGGATCTCGGTGACATTTCT | |
| | GGCATCAACGCTAGCGTAGTGAACATTCAGAAAGAGATAGATAGA | |
| | CTTAATGAGGTGGCCAAGAACCTCAACGAAAGTCTGATCGACCTC | |
| | CAGGAACTGGGGAAATACGAGCAGTACATTAAATGGCCTTGGTAC | |
| | ATATGGCTGGGGTTCATTGCTGGGCTGATCGCAATAGTGATGGTG | |
| | ACCATAATGCTCTGTTGCATGACTAGCTGCTGCAGCTGCCTGAAG | |
| | GGCTGCTGTAGTTGTGGGTCATGTTGTAAGTTTGACGAAGATGAT | |
| | AGCGAGCCTGTCCTTAAAGGAGTGAAGCTCCACTACACCTAG | |
| Spike CT20 | See Sequence Listing | 80 |
| Optimized | | |
| Spike CT56 | See Sequence Listing | 81 |
| Optimized | | |
| Spike CT83 | See Sequence Listing | 82 |
| Optimized | | |
| Spike CT131 | See Sequence Listing | 83 |
| Optimized | | |
| Spike CT199 | See Sequence Listing | 84 |
| Optimized | | |
| Spike CT1-2C | ATGTTTGTCTTCCTGGTCTTGCTGCCGCTcGTGtctAGCCAGTGC | 85 |
| | GTGAATCTCACCACCCGCACCCAGCTTCCACCTGCCTACACTAAC | |
| | AGCTTCACCCGAGGGGTGTATTACCCTGACAAGGTATTCCGGTCC | |
| | TCCGTCCTCCATAGCACGCAGGACCTTTTTCTGCCCTTCTTCTCA | |
| | AATGTGACATGGTTCCATGCCATTCACGTGAGCGGCACGAATGGA | |
| | ACGAAGCGCTTTGATAACCCCGTGCTGCCTTTCAATGACGGCGTC | |
| | TACTTCGCCTCCACTGAAAAGTCAAACATCATCCGGGGCTGGATC | |
| | TTTGGCACCACTCTTGATTCAAAGACCCAGTCACTGCTGATTGTG | |
| | AACAATGCTACAAACGTGGTTATCAAaGTcTGcGAGTTTCAGTTC | |
| | TGTAACGATCCATTTTTGGGAGTGTACTACCACAAGAACAACAAG | |
| | TCCTGGATGGAGTCTGAGTTCAGAGTGTATAGCTCTGCTAACAAC | |
| | TGCACCTTCGAGTACGTGTCCCAGCCTTTCCTTATGGACCTGGAA | |
| | GGCAAACAGGGCAATTTCAAAAACCTGAGAGAGTTCGTGTTTAAG | |
| | AACATTGACGGATACTTCAAAATTTATTCTAAGCACACACCAATT | |
| | AACTTAGTGCGGGACCTACCCCAAGGCTTTAGCGCCCTAGAGCCC | |
| | CTGGTTGACCTGCCCATTGGGATCAATATAACAAGGTTCCAAACT | |
| | CTACTGGCTCTGCATAGAAGTTATCTGACCCCAGGAGACAGCTCT | |
| | AGTGGTTGGACCGCCGGCGCAGCAGCCTACTATGTCGGGTACTTA | |
| | CAGCCACGCACGTTCCTTCTGAAGTACAATGAGAACGGGACAATC | |
| | ACTGACGCAGTAGACTGTGCACTGGACCCGCTAAGCGAGACTAAG | |
| | TGCACACTTAAATCCTTCACGGTGGAGAAAGGCATTTATCAGACC | |
| | TCTAACTTCAGGGTGCAGCCAACAGAAAGCATTGTGCGATTCCCA | |
| | AATATTACTAATCTTTGCCCTTTCGGGGAGGTCTTTAATGCAACT | |
| | AGATTCGCATCAGTCTATGCGTGGAACCGCAAACGCATTTCCAAT | |
| | TGTGTCGCAGACTACTCAGTGCTGTACAACTCTGCCTCTTTCAGT | |
| | ACGTTCAAGTGTTACGGAGTGTCACCCACTAAACTGAACGACCTG | |
| | TGCTTTACAAATGTCTACGCTGACTCCTTCGTGATTAGGGGAGAC | |
| | GAGGTGAGACAAATTGCCCCCGGACAGACTGGGAAGATTGCCGAC | |
| | TACAATTATAAGCTTCCTGATGATTTCACTGGCTGTGTTATTGCC | |
| | TGGAATAGTAACAATCTGGATAGCAAGGTGGGAGGCAACTATAAC | |
| | TACTTATATCGACTGTTTAGGAAGAGTAATCTGAAACCATTTGAG | |
| | CGGGATATTTCCACAGAAATTTACCAGGCCGGGAGCACACCATGT | |
| | AATGGGGTGGAGGGATTTAATTGTTACTTCCCACTCCAGAGCTAT | |
| | GGTTTCCAACCCACCAATGGAGTGGGTTACCAGCCCTATAGAGTC | |
| | GTGGTGCTTAGTTTTGAGCTGCTTCACGCCCCAGCAACCGTCTGC | |
| | GGTCCCAAAAAGTCGACCAATCTCGTGAAAAACAAATGCGTAAAC | |
| | TTCAACTTTAACGGCTTAACAGGAACCGGCGTGCTCACCGAAAGC | |
| | AACAAGAAATTCCTTCCATTTCAGCAATTCGGAAGGGACATCGCC | |
| | GACACAACAGACGCcGTcAGGGACCCACAGACTCTGGAGATACTG | |
| | GACATCACTCCTTGTTCGTTTGGGGGCGTCTCGGTCATCACACCC | |
| | GGGACTAATACTAGTAATCAGGTAGCAGTTTTATATCAAGGCGTC | |
| | AACTGTACCGAAGTACCTGTGGCCATACACGCTGATCAGCTAACG | |
| | CCAACATGGCGAGTCTATTCCACCGGCTCTAACGTTTTTCAGACC | |
| | AGGGCTGGGTGCCTGATAGGGGCAGAGCACGTCAATAATTCCTAT | |
| | GAGTGTGATATCCCCATAGGTGCGGGGATCTGTGCCAGCTATCAA | |
| | ACCCAAACCAATTCACCAAGGCGAGCACGGTCTGTGGCTTCTCAG | |
| | AGCATAATTGCATATACAATGTCACTGGGCGCTGAGAATAGCGTT | |
| | GCATACTCTAATAACAGCATAGCCATTCCCACGAACTTTACTATC | |
| | AGTGTGACAACCGAAATATTGCCAGTTTCGATGACCAAAACTAGC | |
| | GTGGATTGCACGATGTACATCTGTGGAGACTCTACCGAATGCAGC | |
| | AATCTGCTATTACAATATGGCAGCTTCTGTACACAGTTAAATCGA | |
| | GCCTTGACAGGCATCGCAGTGGAACAGGACAAAAATACTCAAGAG | |
| | GTGTTTGCACAGGTGAAGCAAATCTACAAAACGCCCCCCATTAAA | |
| | GATTTTGGCGGGTTCAATTTTTCACAAATTCTCCCCGACCCGTCT | |
| | AAGCCGAGTAAGCGGTCCTTCATCGAAGATCTGCTCTTTAACAAA | |
| | GTAACCCTCGCCGATGCCGGCTTTATTAAGCAGTATGGCGACTGC | |
| | CTGGGGGATATAGCCGCTCGTGACCTGATTTGCGCCCAGAAGTTC | |
| | AATGGTCTGACCGTGTTGCCTCCTTTATTGACCGATGAAATGATT | |
| | GCCCAGTACACTAGTGCCCTGCTGGCCGGCACTATCACGTCTGGG | |
| | TGGACCTTCGGAGCTGGTGCCGCCTTGCAGATACCTTTTGCAATG | |
| | CAGATGGCCTATAGGTTTAATGGTATCGGAGTGACTCAGAACGTA | |
| | CTGTACGAGAACCAGAAGCTCATCGCTAATCAATTTAACTCCGCT | |
| | ATCGGAAAAATCCAGGACAGCCTCTCTTCTACAGCTAGCGCTCTG | |
| | GGCAAACTGCAGGATGTCGTTAATCAGAATGCCCAGGCCCTGAAC | |
| | ACCTTGGTTAAACAACTATCTTCCAACTTCGGGGCCATATCCAGT | |
| | GTGTTGAATGATATTCTCTCCCGCTTGGATAAGGTGGAAGCTGAG | |
| | GTGCAGATCGATCGCTTGATCACCGGCAGACTGCAGTCCCTCCAG | |
| | ACATATGTAACTCAGCAGCTGATTAGAGCCGCCGAGATAAGGGCA | |
| | AGTGCGAATCTGGCTGCCACCAAGATGAGCGAATGTGTATTGGGC | |
| | CAGAGCAAACGAGTTGATTTTTGCGGTAAGGGGTATCATTTAATG | |
| | TCTTTCCCTCAATCCGCACCTCATGGCGTAGTTTTCCTGCATGTG | |
| | ACTTATGTCCCGGCTCAGGAGAAGAATTTTACCACAGCCCCCGCG | |
| | ATCTGCCATGACGGAAAGGCCCACTTCCCCCGGGAAGGCGTGTTT | |
| | GTATCCAATGGGACTCACTGGTTTGTCACTCAGCGAAATTTTTAT | |
| | GAACCACAGATCATCACCACTGACAACACATTTGTTAGTGGAAAC | |
| | TGCGATGTGGTCATCGGCATCGTGAATAACACTGTCTATGATCCA | |
| | CTGCAACCTGAACTGGATTCTTTTAAAGAGGAACTCGACAAGTAT | |
| | TTTAAAAACCACACTAGCCCTGACGTGGATCTCGGTGACATTTCT | |
| | GGCATCAACGCTAGCGTAGTGAACATTCAGAAAGAGATAGATAGA | |
| | CTTAATGAGGTGGCCAAGAACCTCAACGAAAGTCTGATCGACCTC | |
| | CAGGAACTGGGGAAATACGAGCAGTACATTAAATGGCCTTGGTAC | |
| | ATATGGCTGGGGTTCATTGCTGGGCTGATCGCAATAGTGATGGTG | |
| | ACCATAATGCTCTGTTGCATGACTAGCTGCTGCAGCTGCCTGAAG | |
| | GGCTGCTGTAGTTGTGGGTCATGTTGTAAGTTTGACGAAGATGAT | |
| | AGCGAGCCTGTCCTTAAAGGAGTGAAGCTCCACTACACCTAG | |
| Spike IDT-4C | See Sequence Listing | 86 |
| Spike-SGI | See Sequence Listing | 87 |

### Cloning of Sequence-Optimized Spike Sequences

Each sequence-optimized Spike sequence was ordered as a set of 3 gBlocks from IDT with each gblock between 1300-1500 bp and overlapping with each other by approximately 100 nucleotides. The gBlocks comprising the 5′ and 3′ ends of the Spike sequence overlapped with the plasmid backbone by 100 nucleotides. The gblocks were assembled by a combination of PCR and Gibson assembly into a linearized pA68-E4_{d} AsisI/PmeI backbone to generate pA68-E4-sequence-optimized Spike clones. Clones were screened by PCR and clones of the correct size were then grown for plasmid production and sequencing by either NGS or Sanger sequencing. Once a correct clone was sequence confirmed, large scale plasmid production and purification was performed for transfection.

### Vector Production

pA68-E4-Spike plasmid DNA was digested with PacI and 2 ug DNA was transfected into 293F cells using TransIt Lenti transfection reagent. Five days post transfection, cells and media were harvested and a lysate generated by freeze-thawing at −80C and at 37 C. A fraction of the lysate was used to re-infect 30 mL of 293F cells and incubated for 48-72 h before harvesting. Lysate was generated by freeze-thawing at −80C and at 37 C and a fraction of the lysate was used to infect 400 mL of 293F cells seeded at 1e6 cells/mL. Next, 48-72 h later cells were harvested, lysed in 10 mM tris pH 8.0/0.1% Triton X-100 and freeze thawed 1× at 37 and −80C. The lysate was then clarified by centrifugation at 4300×g for 10 min prior to loading on a 1.2/1.4 CsCl gradient. The gradient was run for a minimum of 2 h before the bands were harvested diluted 2-4× in Tris and then rerun on a 1.35 CsCl gradient for at least 2 h. The viral bands were harvested and then dialyzed 3× in 1×ARM buffer. The virus infectious titer was determined by an immunostaining titer assay and the viral particle measured by Absorbance at A260 nm.

### Western Analysis

Samples for Spike expression analysis were either harvested at designated times post transfection or in the case of purified virus by setting up a controlled infection experiment with a known virus MOI and harvested at a specific time post infection, typically 24 to 48 h. 1e6 cells were typically harvested in 0.5 mL of SDS-PAGE loading buffer with 10% Beta-mercaptoethanol. Samples were boiled and run on 4-20% polyacrylamide gels under denaturing and reducing conditions. The gels were then blotted onto a PVDF membrane using a BioRad Rapid transfer device. The membrane was blocked for 2 h at room temperature in 5% Skim milk in TBST. The membrane was then probed with an anti-Spike S1 polyclonal (Sino Biologicals) or anti-Spike monoclonal antibody 1A9 (GeneTex; Cat. No. GTX632604) and incubated for 2 h. The membrane was then washed in PBST (5×) and the probed with a HRP-linked anti-mouse antibody (Bethyl labs) for 1 h. The membrane was washed as described above and then incubated with a chemiluminescent substrate ECL plus (ThermoFisher). The image was then captured using a Chemidoc (BioRad device).

### Results

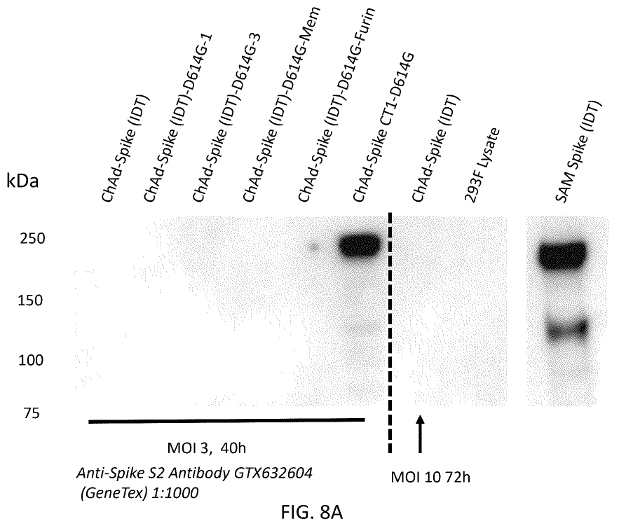

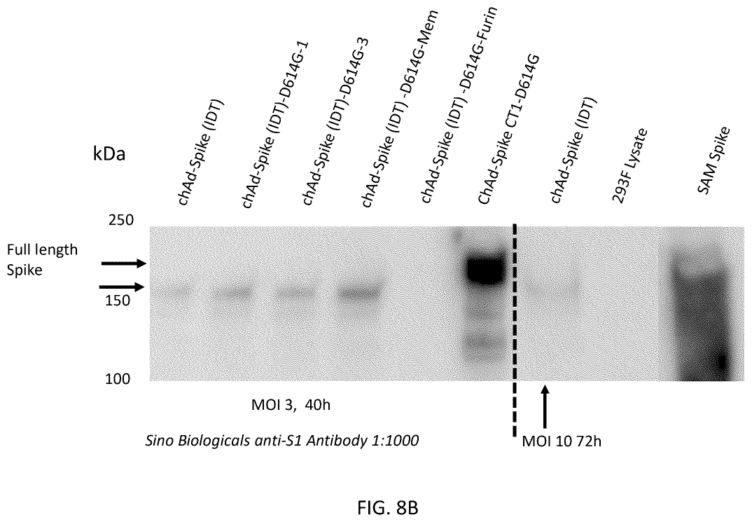

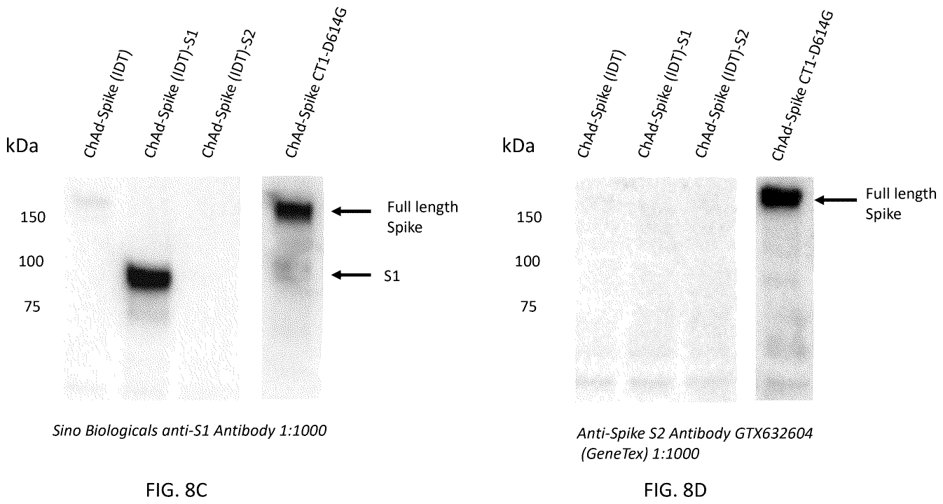

Expression of Spike S2 protein was assessed during viral production in 293F cells with various Spike-encoding vectors. As shown in FIG. 8A, using vectors encoding IDT sequence-optimized Spike cassettes, Spike S2 protein was detected by Western blot using an anti-Spike S2 antibody (GeneTex) when expressed in a SAM vector (FIG. 8A, last lane) but not when expressed in a ChAdV68 vector (“CMV-Spike (IDT)”; SEQ ID NO:69) at two different MOIs and timepoints (FIG. 8A, lanes 1 and 7). Two clones engineered to express Spike variant D614G (“CMV-Spike (IDT)-D614G” SEQ ID NO:70) also did not express detectable levels of Spike protein by Western using the S2 antibody (FIG. 8A, lanes 2 and 3). Clones engineered to co-express the SARS-CoV-2 Membrane protein together with Spike (“CMV-Spike (IDT)-D614G-Mem” SEQ ID NO:66) or including a R682V mutations to disrupt the Furin cleavage site did not rescue the expression phenotype (FIG. 8A, lanes 4 and 5). In contrast, as shown in FIG. 8B, Spike S1 protein was detected for all IDT constructs, albeit at low levels, with the exception of the Furin R682V mutation in which no Spike S1 protein was detected.

To deconvolute if the expressions issues with the IDT sequence-optimized clones were specific to the S1 or S2 domains, vectors expressing only the S1 or S2 domains were also evaluated. As shown in FIG. 8C, a ChAdV68 vector encoding the IDT sequence-optimized Spike S1 protein alone demonstrated strong protein expression, in contrast to the lower expression observed with the full-length Spike vector (FIG. 8C, lanes 1 vs 2). As expected, no signal was observed for S1 with the vector encoding the S2 domain alone. In contrast, as shown in FIG. 8D, a ChAdV68 vector encoding the IDT sequence-optimized Spike S2 protein alone did not demonstrate observable protein expression, comparable to the absence of signal observed with the full-length Spike vector (FIG. 8D, lanes 1 and 3). Thus, the data indicate the IDT sequence-optimized Spike S2 exhibited poor expression, including impacting expression of the full-length Spike sequence.

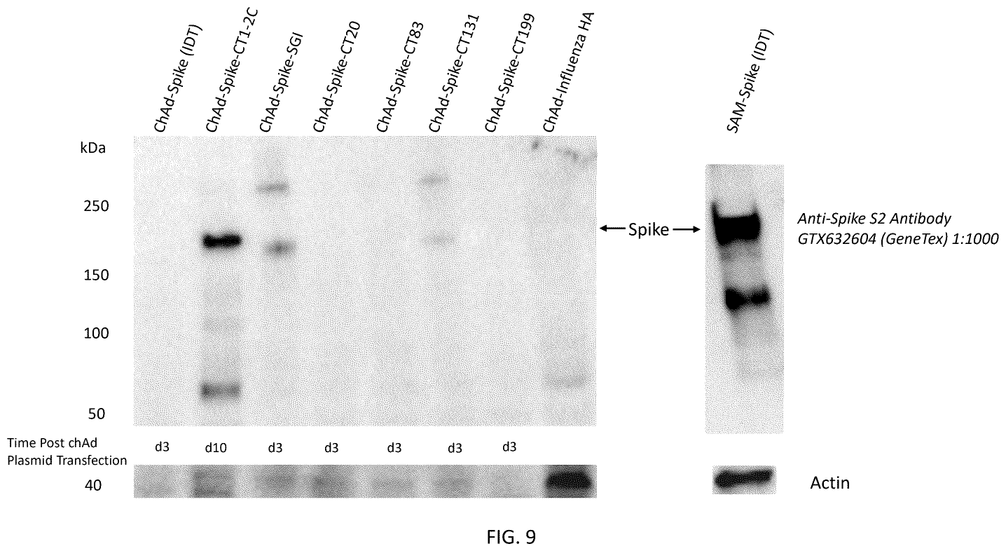

To address protein expression, the SARS-CoV-2 Spike-encoding nucleotide sequence was sequence-optimized using additional sequence-optimization algorithms; (1) a single sequence (SEQ ID NO:87) generated using SGI DNA (La Jolla, CA); (2) 6 sequences designated CT1, CT20, CT56, CT83, CT131, and CT 199 (SEQ ID NOs:79-84) generated using COOL (COOL algorithm generates multiple sequences and 6 were selected). As shown in FIG. 8A and FIG. 8B, sequence-optimization with the COOL algorithm generated a sequence—CT1 (SEQ ID NO:79)—that demonstrated detectable expression using a ChAdV68 vector as assessed by Western using both an anti-S2 and anti-S1 antibody (FIG. 8A and FIG. 8B, each respective lane 6 “ChAd-Spike CT1-D614G”). The additional sequences generated using the COOL algorithm and the SGI algorithm were also assessed by Western. As shown in FIG. 9, the SGI clone and COOL sequence CT131 also demonstrated detectable levels of Spike protein by Western using an anti-S2 antibody (FIG. 9, lanes 3 and 6), while other COOL generated sequences did not generate detectable signals other than the control CT1 derived sequence (lane 2). Thus, the data indicate that specific sequence-optimizations improved expression of full-length SARS-CoV-2 Spike protein in ChAdV68 vectors.

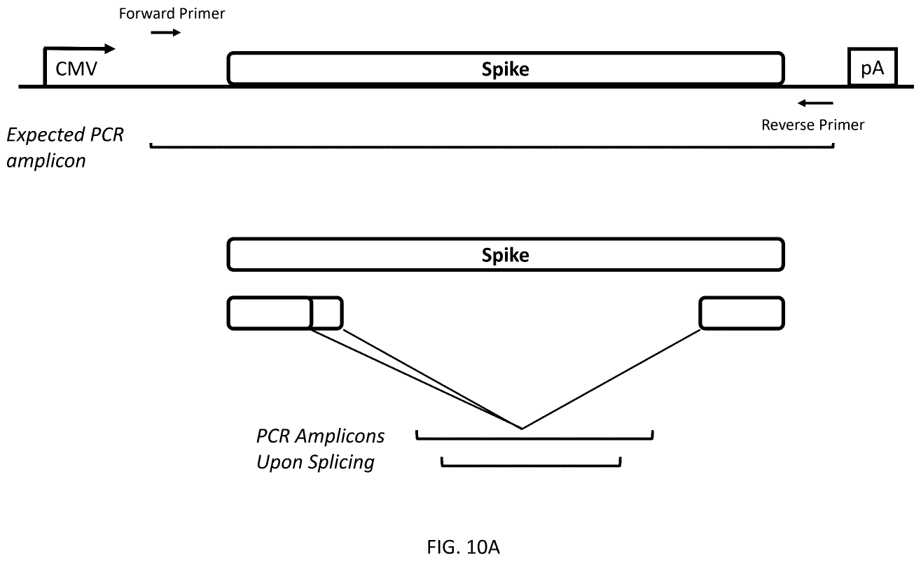

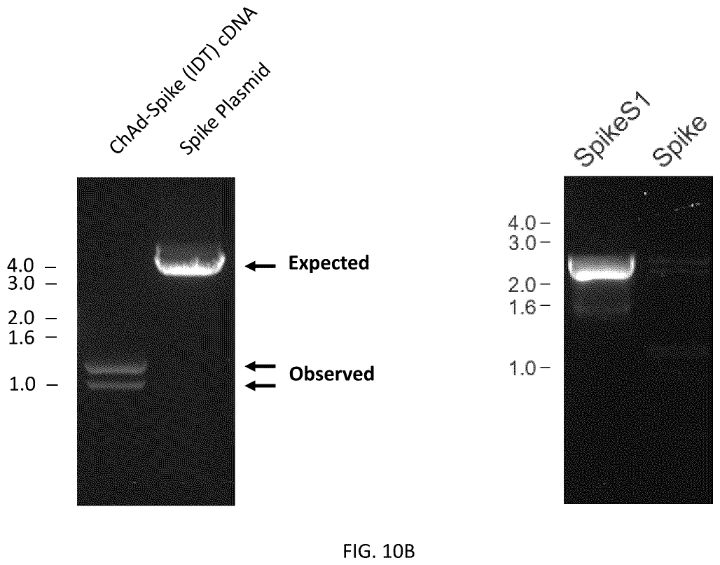

SARS-CoV-2 is a cytoplasm-replicating positive-sense RNA virus encoding its own replication machinery, and as such SARS-CoV-2 mRNA are not naturally processed by splicing and nuclear-export machineries. As illustrated in FIG. 10A, to assess the role of splicing in SARS-CoV-2 Spike-encoding mRNA expressed from a ChAdV68 vector, primers were designed to amplify the Spike coding region. In the presence of mRNA splicing, amplicon sizes would be smaller than the expected full-length coding region. As shown in FIG. 10B, while PCR of the plasmid encoding the SARS-CoV-2 Spike cassette demonstrated the expected amplicon size (“Spike Plasmid” left panel, right column), PCR amplification of cDNA from infected 293 cells demonstrated two smaller amplicons indicating splicing of the mRNA transcript (“ChAd-Spike (IDT) cDNA” left panel, left column). In addition, the Spike coding sequence was split into S1 and S2 encoding sequences. As also shown in FIG. 10B, PCR amplification of S1 cDNA from infected 293 cells demonstrated the expected amplicon size (“SpikeS1” right panel, left column) indicating S1 was likely not undergoing undesired splicing while sequences in the S2 region may be influencing splicing.

The smaller amplicon sequences were analyzed and two splice donor sites were identified by Sanger sequencing. Three additional potential donor sites were predicted by further sequence analysis. The position and identity of the splice motif sequences are presented below (the nt triplets correspond to codons, numbering starts with reference to Spike ATG):

NT 385-:
AAG GTG TGT -> AAa GTc TGc
(identified by sequencing)
NT 539-:
AA GGT AAG C -> Ag GGc AAa C
(identified by sequencing)
NT 2003-:
CA GGT ATCT -> Ct GGa ATC T
(predicted)
NT 2473-:
AAG GTG ACC -> AAa GTc ACC
(predicted)
NT 3417-:
(SEQ ID NO: 123)
C CCC CTT CAG CCT GAA CTT GAT TCC->
(SEQ ID NO: 124)
T CCa CTg CAa CCT GAA CTT GAT agt

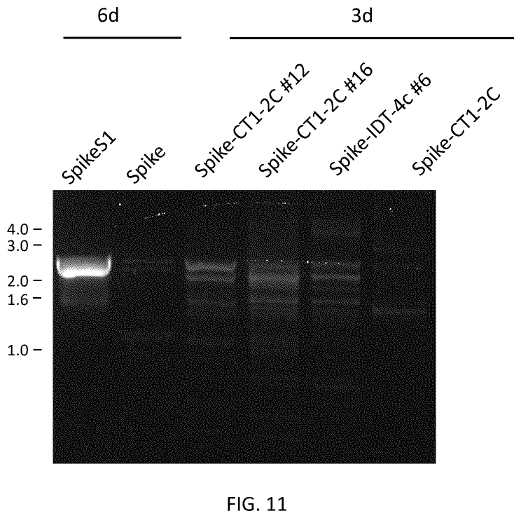

Selected splice donor sites were removed by site-directed mutagenesis disrupting the nucleotide sequence motif while not disturbing the amino acid sequence. COOL sequence-optimized clone CT1 was used as the reference sequence for clone CT1-2C (SEQ ID NO:85) having the sequence-identified splice donor motifs at NT385 and NT539 mutated. IDT sequence-optimized clone was used as the reference sequence for clone IDT-4C (SEQ ID NO:86) and had both sequence-identified and predicted splice donor motifs at NT385, NT539, NT2003, and NT2473 mutated, as well as a possible polyadenylation site AATAAA at NT445 mutated to AAcAAA. As shown in FIG. 9, Spike protein expression was detected by Western in the clone including the sequence-identified splice donor motifs (“CT1-2C” lane 2). Splicing was further assessed in the constructs by PCR analysis. As shown in FIG. 11, mutating the splice donor motifs and/or a potential polyA site alone did not prevent splicing indicating splicing potentially occurred from sub-dominant splice sites.

Given the identification of splicing events in the full-length Spike mRNA expressed from ChAdV68 vectors, additional constructs are generated and assessed for improved protein expression. Additional optimizations include constructs featuring exogenous nuclear export signals (e.g., Constitutive Transport Element (CTE), RNA Transport Element (RTE), or Woodchuck Posttranscriptional Regulatory Element (WPRE)) or the addition of an artificial intron through introduction of exogenous splice donor/branch/acceptor motif sequences to bias splicing, such as introducing a SV40 mini-intron (SEQ ID NO:88) between the CMV promoter and the Kozak sequence immediately upstream of the Spike gene. The identified and predicted splice donor motifs are also further evaluated in combination with additional sequence optimizations.

### XIV.D. SARS-CoV-2 Vaccine Efficacy Evaluation

Various SARS-CoV-2 vaccine designs, constructs, and dosing regimens were evaluated. The vaccines encoded various optimized versions of the Spike protein, selected predicted T cell epitopes (TCE), or a combination of Spike and TCE cassettes.

### Mouse Immunizations

All mouse studies were conducted at Murigenics under IACUC approved protocols. Balb/c mice (Envigo), 6-8 weeks old were used for all studies. Vaccines were stored at −80° C., thawed at room temperature on the day of immunization, and then diluted to 0.1 μg/mL with PBS and filtered through a 0.2 micron filter. Filtered formulations were stored at 4° C. and injected within 4 hours of preparation. All immunizations were bilateral intramuscular to the tibialis anterior, 2 injections of 50 μL each, 100 μL total.

### Non-Human Primate Immunizations

For SAM vaccines in Mamu-A*01 Indian rhesus macaques, SAM was administered as bilateral intramuscular injections into the quadriceps muscle at the indicated doses.

For ChAdV68 vaccines in Mamu-A*01 Indian rhesus macaques, ChAdV68 was administered bilaterally at the indicated doses (5×10¹¹ viral particles per injection).

### Immune Monitoring in Rhesus

For immune monitoring, 10-20 mL of blood was collected into vacutainer tubes containing heparin and maintained at room temperature until isolation. PBMCs were isolated by density gradient centrifugation using lymphocyte separation medium (LSM) and Leucosep separator tubes. PBMCs were stained with propidium iodide and viable cells counted using the Cytoflex LX (Beckman Coulter). Samples were then resuspended at 4×10⁶ cells/mL in RPMI complete (10% FBS).

### Splenocyte Isolation

For the evaluation of T-cell response, mouse spleens were extracted at various timepoints following immunization. Note that in some studies immunizations were staggered to enable spleens to be collected at the same time and compared. Spleens were collected and analyzed by IFNγ ELISpot and ICS. Spleens were suspended in RPMI complete (RPMI+10% FBS) and dissociated using the gentleMACS Dissociator (Miltenyi Biotec). Dissociated cells were filtered using a 40 m strainer and red blood cells were lysed with ACK lysing buffer (150 mM NH₄Cl, 10 mM KHCO₃, 0.1 mM EDTA). Following lysis, cells were filtered with a 30 μm strainer and resuspended in RPMI complete.

### Serum Collection in Mice

At various timepoints post immunization 200 μL of blood was drawn. Blood was centrifuged at 1000 g for 10 minutes at room temperature. Serum was collected and frozen at −80° C.

### S1 IgG MSD/ELISA

96-well QuickPlex plates (Meso Scale Discovery, Rockville, MD) were coated with 50 μL of 1 μg/mL SARS-CoV-2 S1 (ACROBiosystems, Newark, DE), diluted in DPBS (Corning, Corning, NY), and incubated at 4° C. overnight. Wells were washed three times with agitation using 250 μL of PBS+0.05% Tween-20 (Teknova, Hollister, CA) and plates blocked with 150 μL Superblock PBS (Thermo Fisher Scientific, Waltham, MA) for 1 hour at room temperature on an orbital shaker. Test sera was diluted at appropriate series in 10% species-matched serum (Innovative Research, Novi, MI) and tested in single wells on each plate. Starting dilution 1:100, 3-fold dilutions, 11 dilutions per sample. Wells were washed and 50 μL of the diluted samples were added to wells and incubated for 1 hour at room temperature on an orbital shaker. Wells were washed and incubated with 25 μL of 1 μg/mL SULFO-TAG labeled anti-mouse antibody (MSD), diluted in DPBS+1% BSA (Sigma-Aldrich, St. Louis, MO), for 1 hour at room temperature on an orbital shaker. Wells were washed and 150 μL tripropylamine containing read buffer (MSD) added. Plates were run immediately using the QPlex SQ 120 (MSD) ECL plate reader. Endpoint titer is defined as the reciprocal dilution for each sample at which the signal is twice the background value, and is interpolated by fitting a line between the final two values that are greater than twice the background value. The background values is the average value (calculated for each plate) of the control wells containing 10% species-matched serum only.

### Antibody Titers

For antibody response monitoring, antibody titers, including neutralizing antibody titers, in the sera were determined as described in J. Yu et al. (Science 10.1126/science. Abc6284, 2020), herein incorporated by reference for all purposes.

### IFNγ ELISpot Analysis

IFNγ ELISpot assays were performed using pre-coated 96-well plates (MAbtech, Mouse IFNγ ELISpot PLUS, ALP) following manufacturer's protocol. Samples were stimulated overnight with various overlapping peptide pools (15 amino acids in length, 11 amino acid overlap), at a final concentration of 1 μg/mL per peptide. For Spike—eight different overlapping peptide pools spanning the SARS-CoV-2 Spike antigen (Genscript, 36-40 peptides per pool). Splenocytes were plated in duplicate at 1×10⁵ cells per well for each Spike pool, and 2.5×10⁴ cells per well (mixed with 7.5×10⁴ naïve cells) for Spike pools 2, 4, and 7. To measure response to the TCE cassette—one pool spanning Nucleocapsid protein (JPT, NCap-1, 102 peptides), one spanning Membrane protein (JPT, VME-1, 53 peptides), and one spanning the Orf3a regions encoded in the cassette (Genscript, 38 peptides). For TCE peptide pools, splenocytes were plated in duplicate at 2×10⁵ cells per well for each pool. Sequences for peptide pools are presented in Table D (SEQ ID NOS. 27180-27495), Table E (SEQ ID NOS. 27496-27603), and Table F (SEQ ID NOS. 27604-27939). A DMSO only control was plated for each sample and cell number. Following overnight incubation at 37° C., plates were washed with PBS and incubated with anti-monkey IFNγ mAb biotin (MAbtech) for two hours, followed by an additional wash and incubation with Streptavidin-ALP (MAbtech) for one hour. After final wash, plates were incubated for ten minutes with BCIP/NBT (MAbtech) to develop the immunospots. Spots were imaged and enumerated using an AID reader (Autoimmun Diagnostika). For data processing and analysis, samples with replicate well variability (Variability=Variance/(median+1)) greater than 10 and median greater than 10 were excluded. Spot values were adjusted based on the well saturation according to the formula:

AdjustedSpots=RawSpots+2*(RawSpots*Saturation/(100−Saturation)

Each sample was background corrected by subtracting the average value of the negative control peptide wells. Data is presented as spot forming colonies (SFC) per 1×10⁶ splenocytes. Wells with well saturation values greater than 35% were labeled as “too numerous to count” (TNTC) and excluded. For samples and peptides that were TNTC, the value measured with 2.5×10⁴ cells/well was used.

### Vaccine Constructs

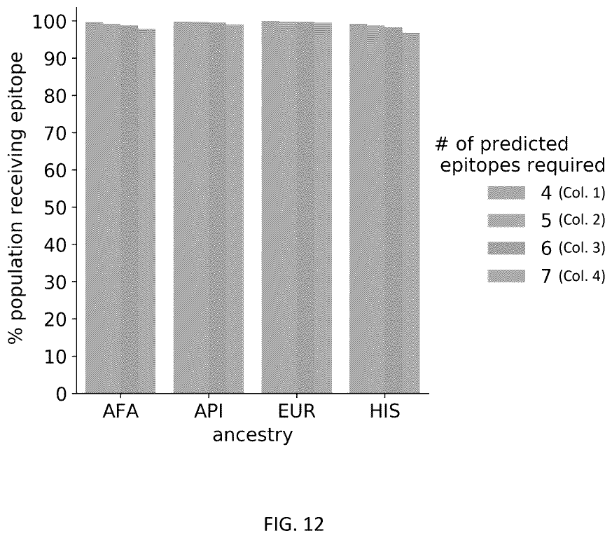

The various sequences evaluated are as follows:
“IDTSpike_{g}”: SARS-CoV-2 Spike protein encoded by IDT optimized sequence (see SEQ ID NO:69) and including a D614G mutation with reference to SEQ ID NO:59 (see corresponding nucleotide mutation in SEQ ID NO:70); also referred to as “Spike V1”
“CTSpike_{g}”: SARS-CoV-2 Spike protein encoded by Cool Tool optimized sequence version 1 (SEQ ID NO:79) including a D614G mutation with reference to SEQ ID NO:59 (see corresponding nucleotide mutation in SEQ ID NO:70); also referred to as “Spike V2.” In versions referred to as “CTSpike_{D}” D614 is not altered.
“CTSpikeF2P_{g}”: SARS-CoV-2 Spike protein encoded by Cool Tool optimized sequence version 1 (SEQ ID NO:79) including a R682V to disrupt the Furin cleavage site (682-685 RRAR [SEQ ID NO: 125] to GSAS [SEQ ID NO: 126]); and K986P and V987P to interfere with the secondary structure of Spike with reference to the reference Spike protein (SEQ ID NO:59). The nucleotide sequence is shown in SEQ ID NO:89 and protein sequence shown in SEQ ID NO:90
“TCE5”: Selected CD8+ epitopes predicted by the EDGE platform to be presented on MHC molecules for SARS-CoV-2 proteins other than Spike. The 15 selected epitopes are presented along with their order in the cassette in Table 7. The nucleotide sequence is shown in SEQ ID NO:91 and protein sequence shown in SEQ ID NO:92. FIG. 12 shows the estimated protection across the four indicated populations for TCE5. All populations are estimated to have coverage above 95% up to at least the threshold of 7 epitopes (last column).
The SAM vector SAM-SGP1-TCE5-SGP2-CTSpike_{G}F2P is shown in SEQ ID NO:93
The ChAd vector ChAd-CMV-CTSpike_{G}F2P-CMV-TCE5 (EPE) is shown in SEQ ID NO: 114
Additional vectors and Spike variants were designed for evaluation as shown in SEQ ID NOs:109-113

TABLE 6
Encoded Spike Variants

CTSpikeF2Pg nucleotide (SEQ ID NO: 89);
Bold Italic Furin Mutation 682-685
RRAR (SEQ ID NO: 125) to GSAS
(SEQ ID NO: 126), Bold Lower Case K986P
and V987P
ATGTTTGTCTTCCTGGTCTTGCTGCCGCTGGTGAGCAGCCAGTGC
GTGAATCTCACCACCCGCACCCAGCTTCCACCTGCCTACACTAAC
AGCTTCACCCGAGGGGTGTATTACCCTGACAAGGTATTCCGGTCC
TCCGTCCTCCATAGCACGCAGGACCTTTTTCTGCCCTTCTTCTCA
AATGTGACATGGTTCCATGCCATTCACGTGAGCGGCACGAATGGA
ACGAAGCGCTTTGATAACCCCGTGCTGCCTTTCAATGACGGCGTC
TACTTCGCCTCCACTGAAAAGTCAAACATCATCCGGGGCTGGATC
TTTGGCACCACTCTTGATTCAAAGACCCAGTCACTGCTGATTGTG
AACAATGCTACAAACGTGGTTATCAAGGTGTGTGAGTTTCAGTTC
TGTAACGATCCATTTTTGGGAGTGTACTACCACAAGAACAACAAG
TCCTGGATGGAGTCTGAGTTCAGAGTGTATAGCTCTGCTAACAAC
TGCACCTTCGAGTACGTGTCCCAGCCTTTCCTTATGGACCTGGAA
GGCAAACAGGGCAATTTCAAAAACCTGAGAGAGTTCGTGTTTAAG
AACATTGACGGATACTTCAAAATTTATTCTAAGCACACACCAATT
AACTTAGTGCGGGACCTACCCCAAGGCTTTAGCGCCCTAGAGCCC
CTGGTTGACCTGCCCATTGGGATCAATATAACAAGGTTCCAAACT
CTACTGGCTCTGCATAGAAGTTATCTGACCCCAGGAGACAGCTCT
AGTGGTTGGACCGCCGGCGCAGCAGCCTACTATGTCGGGTACTTA
CAGCCACGCACGTTCCTTCTGAAGTACAATGAGAACGGGACAATC
ACTGACGCAGTAGACTGTGCACTGGACCCGCTAAGCGAGACTAAG
TGCACACTTAAATCCTTCACGGTGGAGAAAGGCATTTATCAGACC
TCTAACTTCAGGGTGCAGCCAACAGAAAGCATTGTGCGATTCCCA
AATATTACTAATCTTTGCCCTTTCGGGGAGGTCTTTAATGCAACT
AGATTCGCATCAGTCTATGCGTGGAACCGCAAACGCATTTCCAAT
TGTGTCGCAGACTACTCAGTGCTGTACAACTCTGCCTCTTTCAGT
ACGTTCAAGTGTTACGGAGTGTCACCCACTAAACTGAACGACCTG
TGCTTTACAAATGTCTACGCTGACTCCTTCGTGATTAGGGGAGAC
GAGGTGAGACAAATTGCCCCCGGACAGACTGGGAAGATTGCCGAC
TACAATTATAAGCTTCCTGATGATTTCACTGGCTGTGTTATTGCC
TGGAATAGTAACAATCTGGATAGCAAGGTGGGAGGCAACTATAAC
TACTTATATCGACTGTTTAGGAAGAGTAATCTGAAACCATTTGAG
CGGGATATTTCCACAGAAATTTACCAGGCCGGGAGCACACCATGT
AATGGGGTGGAGGGATTTAATTGTTACTTCCCACTCCAGAGCTAT
GGTTTCCAACCCACCAATGGAGTGGGTTACCAGCCCTATAGAGTC
GTGGTGCTTAGTTTTGAGCTGCTTCACGCCCCAGCAACCGTCTGC
GGTCCCAAAAAGTCGACCAATCTCGTGAAAAACAAATGCGTAAAC
TTCAACTTTAACGGCTTAACAGGAACCGGCGTGCTCACCGAAAGC
AACAAGAAATTCCTTCCATTTCAGCAATTCGGAAGGGACATCGCC
GACACAACAGACGCGGTGAGGGACCCACAGACTCTGGAGATACTG
GACATCACTCCTTGTTCGTTTGGGGGCGTCTCGGTCATCACACCC
GGGACTAATACTAGTAATCAGGTAGCAGTTTTATATCAAGGCGTC
AACTGTACCGAAGTACCTGTGGCCATACACGCTGATCAGCTAACG
CCAACATGGCGAGTCTATTCCACCGGCTCTAACGTTTTTCAGACC
AGGGCTGGGTGCCTGATAGGGGCAGAGCACGTCAATAATTCCTAT
GAGTGTGATATCCCCATAGGTGCGGGGATCTGTGCCAGCTATCAA
ACCCAAACCAATTCACCAgGGaGcGCAaGcTCTGTGGCTTCTCAG
AGCATAATTGCATATACAATGTCACTGGGCGCTGAGAATAGCGTT
GCATACTCTAATAACAGCATAGCCATTCCCACGAACTTTACTATC
AGTGTGACAACCGAAATATTGCCAGTTTCGATGACCAAAACTAGC
GTGGATTGCACGATGTACATCTGTGGAGACTCTACCGAATGCAGC
AATCTGCTATTACAATATGGCAGCTTCTGTACACAGTTAAATCGA
GCCTTGACAGGCATCGCAGTGGAACAGGACAAAAATACTCAAGAG
GTGTTTGCACAGGTGAAGCAAATCTACAAAACGCCCCCCATTAAA
GATTTTGGCGGGTTCAATTTTTCACAAATTCTCCCCGACCCGTCT
AAGCCGAGTAAGCGGTCCTTCATCGAAGATCTGCTCTTTAACAAA
GTAACCCTCGCCGATGCCGGCTTTATTAAGCAGTATGGCGACTGC
CTGGGGGATATAGCCGCTCGTGACCTGATTTGCGCCCAGAAGTTC
AATGGTCTGACCGTGTTGCCTCCTTTATTGACCGATGAAATGATT
GCCCAGTACACTAGTGCCCTGCTGGCCGGCACTATCACGTCTGGG
TGGACCTTCGGAGCTGGTGCCGCCTTGCAGATACCTTTTGCAATG
CAGATGGCCTATAGGTTTAATGGTATCGGAGTGACTCAGAACGTA
CTGTACGAGAACCAGAAGCTCATCGCTAATCAATTTAACTCCGCT
ATCGGAAAAATCCAGGACAGCCTCTCTTCTACAGCTAGCGCTCTG
GGCAAACTGCAGGATGTCGTTAATCAGAATGCCCAGGCCCTGAAC
ACCTTGGTTAAACAACTATCTTCCAACTTCGGGGCCATATCCAGT
GTGTTGAATGATATTCTCTCCCGCTTGGATccacctGAAGCTGAG
GTGCAGATCGATCGCTTGATCACCGGCAGACTGCAGTCCCTCCAG
ACATATGTAACTCAGCAGCTGATTAGAGCCGCCGAGATAAGGGCA
AGTGCGAATCTGGCTGCCACCAAGATGAGCGAATGTGTATTGGGC
CAGAGCAAACGAGTTGATTTTTGCGGTAAGGGGTATCATTTAATG
TCTTTCCCTCAATCCGCACCTCATGGCGTAGTTTTCCTGCATGTG
ACTTATGTCCCGGCTCAGGAGAAGAATTTTACCACAGCCCCCGCG
ATCTGCCATGACGGAAAGGCCCACTTCCCCCGGGAAGGCGTGTTT
GTATCCAATGGGACTCACTGGTTTGTCACTCAGCGAAATTTTTAT
GAACCACAGATCATCACCACTGACAACACATTTGTTAGTGGAAAC
TGCGATGTGGTCATCGGCATCGTGAATAACACTGTCTATGATCCA
CTGCAACCTGAACTGGATTCTTTTAAAGAGGAACTCGACAAGTAT
TTTAAAAACCACACTAGCCCTGACGTGGATCTCGGTGACATTTCT
GGCATCAACGCTAGCGTAGTGAACATTCAGAAAGAGATAGATAGA
CTTAATGAGGTGGCCAAGAACCTCAACGAAAGTCTGATCGACCTC
CAGGAACTGGGGAAATACGAGCAGTACATTAAATGGCCTTGGTAC
ATATGGCTGGGGTTCATTGCTGGGCTGATCGCAATAGTGATGGTG
ACCATAATGCTCTGTTGCATGACTAGCTGCTGCAGCTGCCTGAAG
GGCTGCTGTAGTTGTGGGTCATGTTGTAAGTTTGACGAAGATGAT
AGCGAGCCTGTCCTTAAAGGAGTGAAGCTCCACTACACCTAG
CTSpikeF2Pg amino acid (SEQ ID NO: 90);
Bold Italic Furin Mutation 682-685 RRAR
(SEQ ID NO: 125) to GSAS (SEQ ID NO: 126),
Bold Lower Case K986P and V987P
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVENATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGENCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQGVNCTEVPVAIHADQLT
PTWRVYSTGSNVFQTRAGCLIGAEHVNNSYECDIPIGAGICASYQ
TQTNSPGSASSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSFIEDLLFNKVTLADAGFIKQYGDCLGDIAARDLICAQKF
NGLTVLPPLLTDEMIAQYTSALLAGTITSGWTFGAGAALQIPFAM
QMAYRENGIGVTQNVLYENQKLIANQFNSAIGKIQDSLSSTASAL
GKLQDVVNQNAQALNTLVKQLSSNFGAISSVLNDILSRLDppEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQYIKWPWY
IWLGFIAGLIAIVMVTIMLCCMTSCCSCLKGCCSCGSCCKFDEDD
SEPVLKGVKLHYT*

TABLE 7
TCE5 Cassette (Order of Frames as Shown)

| | | | | SEQ | |
|---|---|---|---|---|---|
| | | Start | End | ID | |
| Frame | Gene | aa | aa | NO | Amino Acid Sequence |
| 1 | ORF3a | 102 | 152 | 94 | EAPFLYLYALVYFLQSINFVRIIMRLWLCWKCRSKNPLL |
| | | | | | YDANYFLCWHTN |
| 2 | ORF3a | 53 | 85 | 95 | LAVFQSASKIITLKKRWQLALSKGVHFVCNLLL |
| 3 | ORF3a | 13 | 55 | 96 | VTLKQGEIKDATPSDFVRATATIPIQASLPFGWLIVGVAL |
| | | | | | LAV |
| 4 | N | 40 | 94 | 97 | RRPQGLPNNTASWFTALTQHGKEDLKFPRGQGVPINTN |
| | | | | | SSPDDQIGYYRRATRRI |
| 5 | M | 84 | 102 | 98 | MACLVGLMWLSYFIASFRL |
| 6 | N | 369 | 408 | 99 | KKDKKKKADETQALPQRQKKQQTVTLLPAADLDDESK |
| | | | | | QLQ |
| 7 | N | 284 | 338 | 100 | GNFGDQELIRQGTDYKHWPQIAQFAPSASAFFGMSRIG |
| | | | | | MEVTPSGTWLTYTGAIK |
| 8 | ORF3a | 169 | 198 | 101 | ITSGDGTTSPISEHDYQIGGYTEKWESGVK |
| 9 | N | 233 | 282 | 102 | KMSGKGQQQQGQTVTKKSAAEASKKPRQKRTATKAY |
| | | | | | NVTQAFGRRGPEQT |
| 10 | M | 56 | 75 | 103 | LLWPVTLACFVLAAVYRINW |
| 11 | N | 346 | 375 | 104 | FKDQVILLNKHIDAYKTFPPTEPKKDKKKK |
| 12 | N | 205 | 239 | 105 | TSPARMAGNGGDAALALLLLDRLNQLESKMSGKGQ |
| 13 | N | 100 | 118 | 106 | KMKDLSPRWYFYYLGTGPE |
| 14 | ORF3a | 199 | 254 | 107 | DCVVLHSYFTSDYYQLYSTQLSTDTGVEHVTFFIYNKIV |
| | | | | | DEPEEHVQIHTIDGSSG |
| 15 | N | 129 | 174 | 108 | GIIWVATEGALNTPKDHIGTRNPANNAAIVLQLPQGTTL |
| | | | | | PKGFYAE |

TCE5 Nucleotide Sequence (SEQ ID NO: 91):
ATGGCTGGCGAGGCCCCCTTCCTTTACCTGTACGCCCTTGTGTAT
TTCCTGCAGAGCATCAATTTTGTGAGAATCATCATGAGGCTGTGG
CTTTGCTGGAAATGTAGGAGCAAGAACCCCCTGTTGTATGACGCC
AACTACTTTCTGTGTTGGCACACCAATCTCGCCGTGTTCCAGAGT
GCCTCTAAGATCATTACACTGAAAAAGCGGTGGCAGCTTGCACTT
TCTAAGGGAGTGCATTTCGTTTGCAACCTGCTCCTGGTGACACTC
AAGCAGGGGGAAATCAAAGACGCCACCCCTAGCGACTTCGTTAGA
GCCACTGCCACAATCCCAATCCAGGCTTCCCTGCCTTTCGGCTGG
CTTATCGTGGGTGTGGCACTGTTGGCTGTGCGGAGACCACAGGGA
CTGCCTAATAATACAGCTAGCTGGTTTACCGCTCTGACACAGCAT
GGCAAAGAAGACCTCAAGTTCCCTCGCGGTCAGGGGGTGCCTATT
AACACTAATAGCTCTCCAGACGACCAAATTGGGTATTACAGGCGC
GCCACAAGACGGATCATGGCCTGCTTAGTGGGGCTGATGTGGCTA
TCCTATTTTATTGCTAGCTTTCGCCTGAAGAAGGACAAGAAGAAG
AAAGCTGATGAGACCCAGGCACTGCCCCAGCGCCAAAAGAAGCAG
CAGACAGTCACACTGCTCCCTGCTGCAGACCTGGATGACTTCAGC
AAGCAGCTGCAGGGGAACTTTGGCGACCAGGAGCTGATTAGACAG
GGGACTGACTATAAGCATTGGCCTCAGATTGCTCAGTTCGCCCCA
AGTGCATCCGCCTTCTTCGGGATGTCACGAATAGGAATGGAAGTG
ACCCCTTCTGGGACATGGTTGACATACACCGGAGCAATCAAGATT
ACCTCCGGGGACGGTACCACGTCTCCTATTAGCGAACACGATTAT
CAGATAGGGGGATATACTGAGAAGTGGGAGTCCGGCGTCAAAAAG
ATGAGTGGGAAAGGCCAGCAGCAACAAGGCCAAACAGTTACTAAA
AAGTCTGCCGCAGAAGCTAGTAAAAAGCCTCGCCAGAAGCGGACA
GCCACCAAAGCTTACAATGTGACTCAGGCCTTCGGCCGCCGGGGG
CCTGAACAGACCCTCTTGTGGCCCGTTACCCTCGCATGTTTCGTG
CTTGCAGCTGTGTACAGGATCAATTGGTTTAAGGATCAGGTTATC
CTGTTGAACAAACATATAGATGCCTATAAGACATTCCCACCCACC
GAGCCAAAGAAAGATAAGAAAAAGAAAACTAGTCCTGCAAGGATG
GCCGGCAATGGAGGAGACGCAGCCTTAGCCCTGCTCTTACTCGAC
AGGCTGAACCAACTTGAGTCTAAAATGAGCGGTAAAGGGCAGAAG
ATGAAGGATCTGTCCCCAAGGTGGTATTTCTACTATCTGGGCACC
GGCCCTGAGGATTGTGTCGTCCTCCACTCATACTTCACTAGCGAT
TATTACCAGCTGTATAGTACACAATTATCTACCGACACAGGCGTC
GAGCACGTGACCTTCTTTATATACAATAAGATCGTGGATGAACCA
GAGGAGCATGTGCAGATCCACACTATTGATGGCTCTAGCGGGGGC
ATCATCTGGGTGGCAACAGAAGGAGCCCTCAACACCCCAAAGGAC
CATATCGGCACCAGGAATCCAGCCAACAATGCCGCCATTGTTCTG
CAGCTCCCTCAGGGCACTACTCTCCCTAAAGGCTTCTATGCTGAG
GGACCCGGACCAGGCGCCAAATTTGTTGCTGCTTGGACACTGAAA
GCTGCTGCTGGGCCCGGACCAGGCCAGTACATCAAGGCCAACTCT
AAGTTTATCGGCATCACCGAATTGGGACCTGGACCCGGCTAG
TCE5 Amino Acid Sequence (SEQ ID NO: 92):
MAGEAPFLYLYALVYFLQSINFVRIIMRLWLCWKCRSKNPLLYDA
NYFLCWHTNLAVFQSASKIITLKKRWQLALSKGVHFVCNLLLVTL
KQGEIKDATPSDFVRATATIPIQASLPFGWLIVGVALLAVRRPQG
LPNNTASWFTALTQHGKEDLKFPRGQGVPINTNSSPDDQIGYYRR
ATRRIMACLVGLMWLSYFIASFRLKKDKKKKADETQALPQRQKKQ
QTVTLLPAADLDDFSKQLQGNFGDQELIRQGTDYKHWPQIAQFAP
SASAFFGMSRIGMEVTPSGTWLTYTGAIKITSGDGTTSPISEHDY
QIGGYTEKWESGVKKMSGKGQQQQGQTVTKKSAAEASKKPRQKRT
ATKAYNVTQAFGRRGPEQTLLWPVTLACFVLAAVYRINWFKDQVI
LLNKHIDAYKTFPPTEPKKDKKKKTSPARMAGNGGDAALALLLLD
RLNQLESKMSGKGQKMKDLSPRWYFYYLGTGPEDCVVLHSYFTSD
YYQLYSTQLSTDTGVEHVTFFIYNKIVDEPEEHVQIHTIDGSSGG
IIWVATEGALNTPKDHIGTRNPANNAAIVLQLPQGTTLPKGFYAE
GPGPGAKFVAAWTLKAAAGPGPGQYIKANSKFIGITELGPGPG-

SAM-SGP1-TCE5-SGP2-CTSpike_{G}F2P Nucleotide Sequence in vitro transcription template DNA sequence (SEQ ID NO:93): (NT 1-17: T7 promoter; NT 62-7543: VEEV non-structural protein coding region; NT 7518-7560: SGP1; NT 7582-7587 and 9541-9546: Kozak sequence; NT 7588-9474: TCE5 cassette; NT 9480-9540: SGP2; NT 9547-13368: CTSpike Furin-2P).

ChAd-CMV-CTSpike_{G}F2P-CMV-TCE5 (EPE) Nucleotide Sequence (SEQ ID NO:114); See Sequence Listing.

### XIV.E. SARS-CoV-2 Vaccine Produces Responses to Various Spike Constructs

ChAd and SAM vaccine platforms encoding various versions of the SARS-CoV-2 Spike protein were assessed.

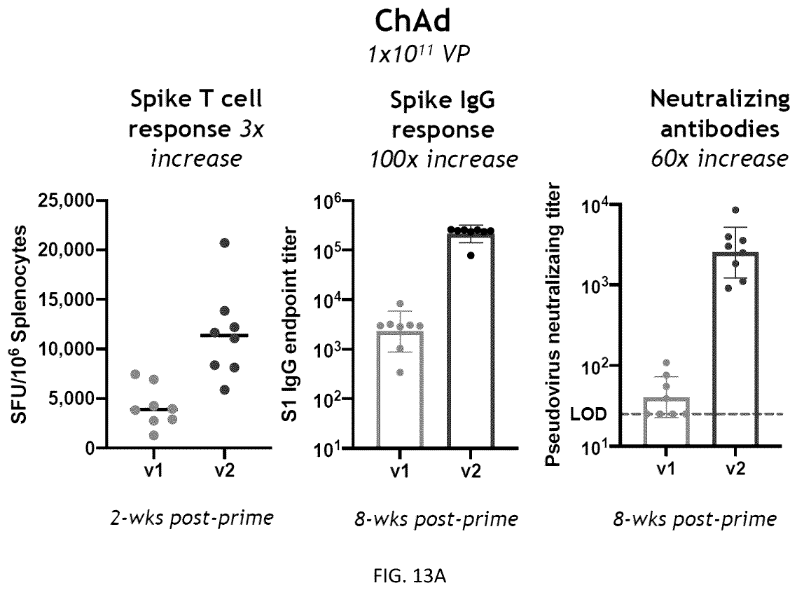

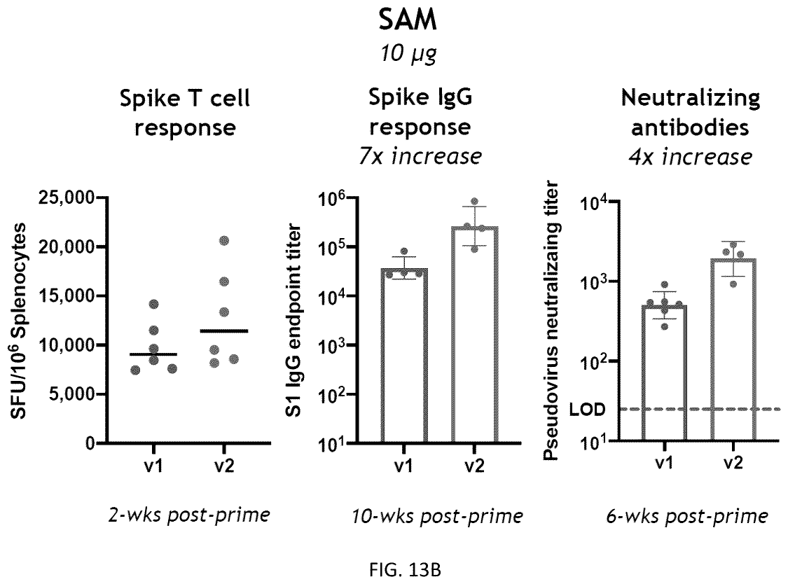

Versions of a Spike-encoding cassette featuring different sequence optimizations were assessed: “IDTSpike_{g}” (SEQ ID NO:69, also referred to as “Spike V1” or “v1”); “CTSpike_{g}”: (SEQ ID NO:79, also referred to as “Spike V2” or “v2”). As shown in FIG. 13, both ChAd (FIG. 13A) and SAM (FIG. 13B) vaccines produced detectable T cell responses (left panel), Spike-specific IgG antibodies (middle panel) and neutralizing antibodies (right panel). Notably, a ChAd vaccine encoding the CTSpike_{g} sequence version produced a 3-fold increased T cell response, 100-fold increased IgG production, and 60-fold increase in neutralizing antibody titer. Similarly, a SAM vaccine encoding the CTSpike_{g} sequence version produced an increased T cell response, 7-fold increase in IgG production, and 4-fold increase in neutralizing antibody titer. Accordingly, the data demonstrate sequence optimization of the Spike cassette produced an increased immune response across the multiple parameters assessed for each vaccine platform examined.

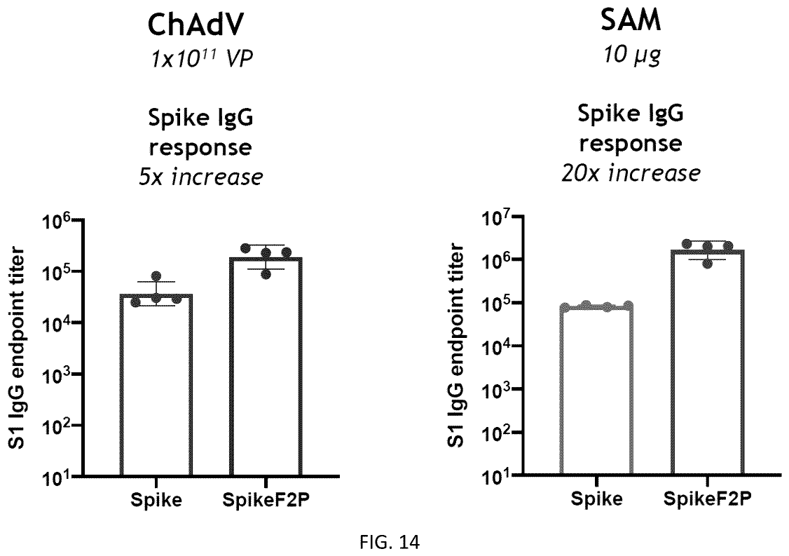

A version of a Spike-encoding cassette featuring modified Spike that includes removal of a furin site and addition of prolines in S2 domain was assessed: “CTSpikeF2P_{g}” (SEQ ID NO:89 and SEQ ID NO:90). As shown in FIG. 14, both ChAd (left panel) and SAM (right panel) vaccines encoding the F2P-modified Spike produced 5-fold and 20-fold Spike-specific IgG antibodies, respectively, relative to a corresponding “CTSpike_{g}” cassette that does not have the referenced modifications. Accordingly, the data demonstrate modification of the Spike cassette produced an increased antibody response for each vaccine platform examined.

### XIV.F. SARS-CoV-2 Vaccine Produces Responses to T Cell Epitopes

ChAd and SAM vaccine platforms encoding various a modified SARS-CoV-2 Spike protein and a T cell epitope (TCE) cassette encoding EDGE predicted epitopes (EPE) were assessed.

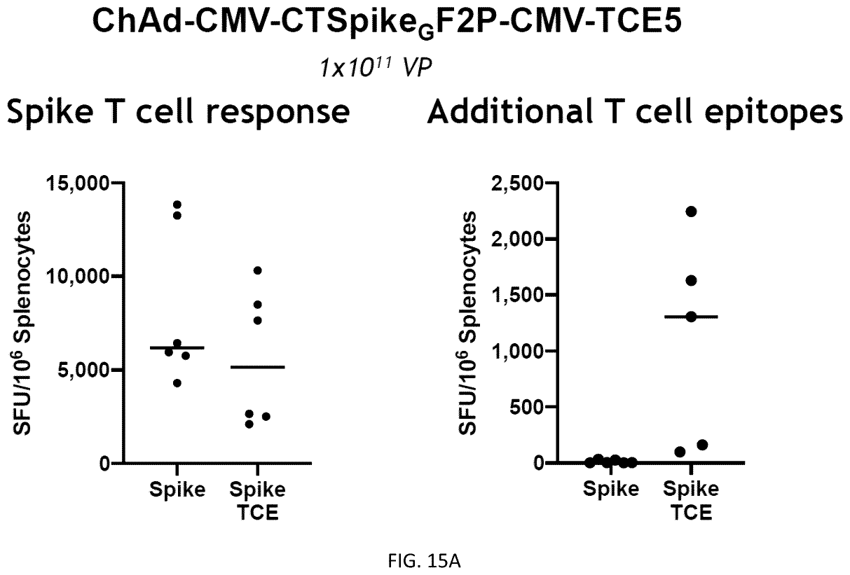

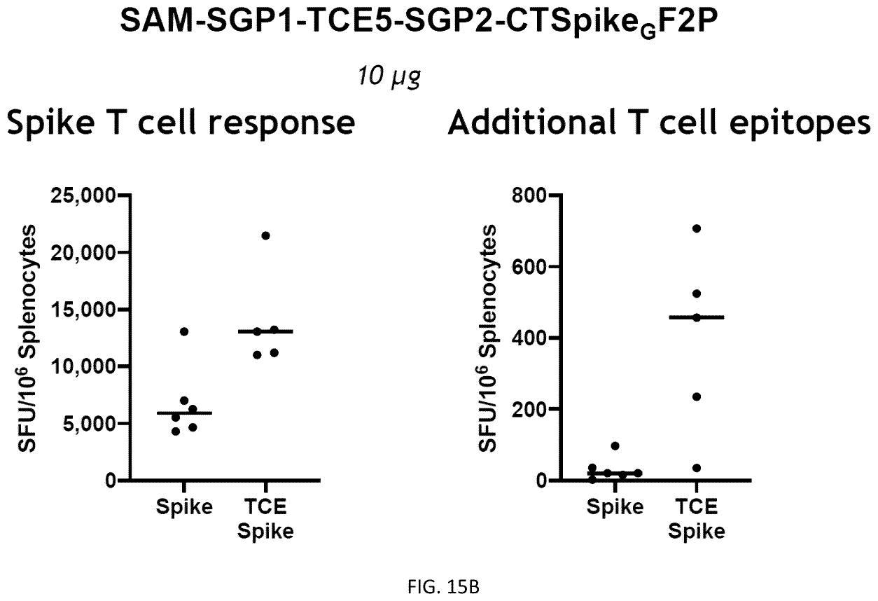

Both a modified Spike-encoding only cassette (“CTSpikeF2P_{g}” (SEQ ID NO:89) and modified Spike together with additional non-Spike T cell epitopes (ChAd SEQ ID NO:114; SAM SEQ ID NO:93; see Table 7 for “TCE5”), and immune responses assessed, as described above. As shown in FIG. 15, both ChAd (FIG. 15A) and SAM (FIG. 15B) each vaccine assessed produced detectable T cell responses to Spike (left panel), while vaccines including the TCE5 cassette also generally produced detectable T cell responses to the encoded T cell epitopes (right panel). Accordingly, the data demonstrate addition of a T cell epitope cassette led to broad T cell responses across the SARS-CoV-2 Genome for each vaccine platform examined.

### XIV.G. Order of Cassettes in SARS-CoV-2 Vaccine Influences Immune Response

SAM vaccine platforms encoding various orders of a modified SARS-CoV-2 Spike protein and a T cell epitope (TCE) cassette encoding EDGE predicted epitopes (EPE) were assessed.

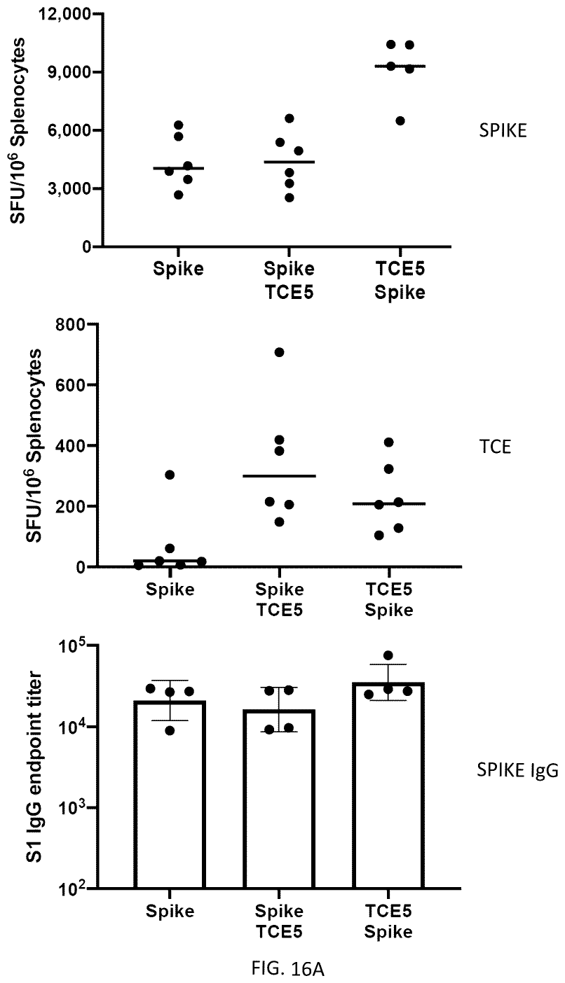

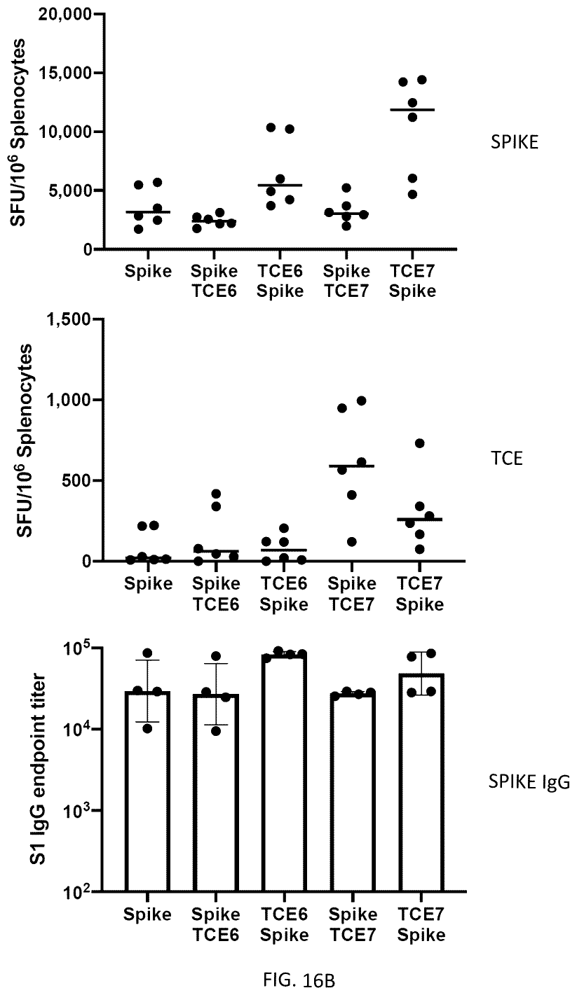

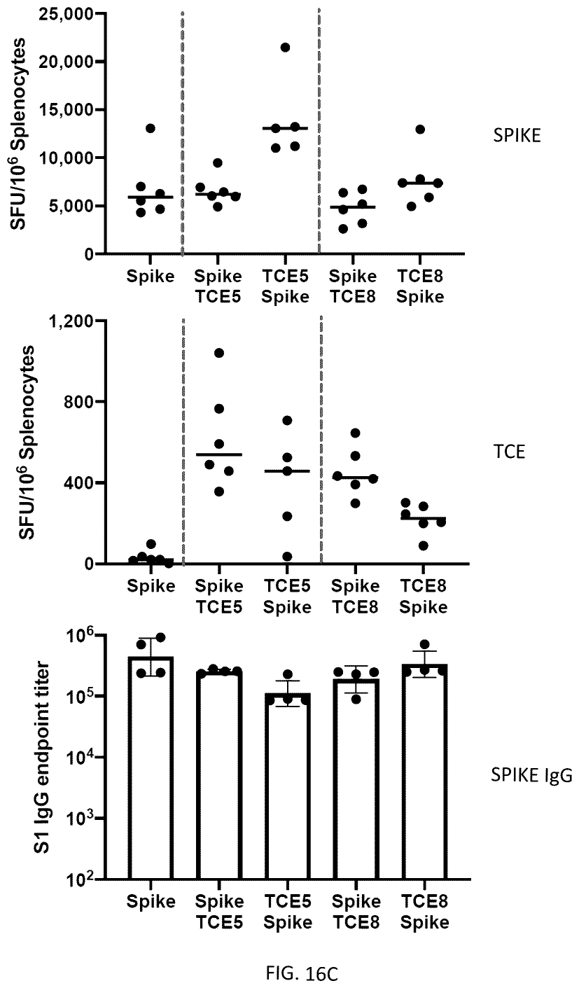

As shown in FIG. 16, T cell responses to Spike (top panel), T cell responses to the encoded T cell epitopes (middle panel), and Spike-specific IgG antibodies (bottom panel) were produced when vaccinated with various constructs. In FIG. 16A, SAM constructs included “IDTSpike_{g}” (SEQ ID NO:69) alone (left columns), IDTSpike_{g} expressed from a first subgenomic promoter followed by TCE5 expressed from a second subgenomic promoter (middle columns), or TCE5 expressed from a first subgenomic promoter followed by IDTSpike_{g} expressed from a second subgenomic promoter (right columns), with immune responses assessed, as described above. In FIG. 16B, SAM constructs included “IDTSpike_{g}” (SEQ ID NO:69) alone (first column), IDTSpike_{g} expressed from a first subgenomic promoter followed by TCE6 or TCE7 expressed from a second subgenomic promoter (columns 2 and 4, respectively), or TCE6 or TCE7 expressed from a first subgenomic promoter followed by IDTSpike_{g} expressed from a second subgenomic promoter (columns 3 and 5, respectively), with immune responses assessed, as described above. In FIG. 16C, SAM constructs included “CTSpike_{g}” (SEQ ID NO:79) alone (first column), CTSpike_{g} expressed from a first subgenomic promoter followed by TCE5 or TCE8 expressed from a second subgenomic promoter (columns 2 and 4, respectively), or TCE5 or TCE8 expressed from a first subgenomic promoter followed by CTSpike_{g} expressed from a second subgenomic promoter (columns 3 and 5, respectively), with immune responses assessed, as described above. Generally, and in particular for Spike, T cell responses were increased when the respective epitopes were expressed from the second subgenomic promoter, including increased Spike-directed T cell responses relative to Spike alone. A similar trend was also observed generally observed with increased Spike-specific IgG titers when the Spike antigen was expressed from the second subgenomic promoter except, for potentially the CTSpike_{g} constructs. Accordingly, the data demonstrate sequence order of antigen cassettes in vaccine platforms influenced immune responses.

### XIV.H. Additional SARS-CoV-2 Vaccine Constructions

Vaccines were constructed to maximize the percentage of people getting predicted to achieve a total magnitude of greater than 1000 from validated epitopes. Briefly, magnitude was calculated across all validated epitopes in the starting proteins (e.g., Spike), as well as any added in the TCE cassettes according to the following with an expected approximate upper size limit of 600 amino acids beyond that of Spike: (1) An individual's magnitude is the sum of all epitope magnitudes across their respective diplotype alleles; (2) Each epitopes magnitude=(magnitude of response)×(Frequency of positive response/100), with values found in Tarke et al. (Comprehensive analysis of T cell immunodominance and immunoprevalence of SARS-CoV-2 epitopes in COVID-19 cases. Cell Rep Med. 2021 Feb. 16; 2(2):100204. doi: 10.1016/j.xcrm.2021.100204. Epub 2021 Jan. 26.), herein incorporated by reference for all purposes; (3) epitopes other than those from starting proteins that span mutations with >5% frequency were excluded (see Table 8 for all mutations >1% frequency either overall or in specific strains), though mutations are allowed in flanking regions; and (4) cassette order was chosen to minimize unintended junction epitopes across adjacent frames, as well as minimize consecutive frames in the same protein to reduce chance of functional protein fragments, as described above.

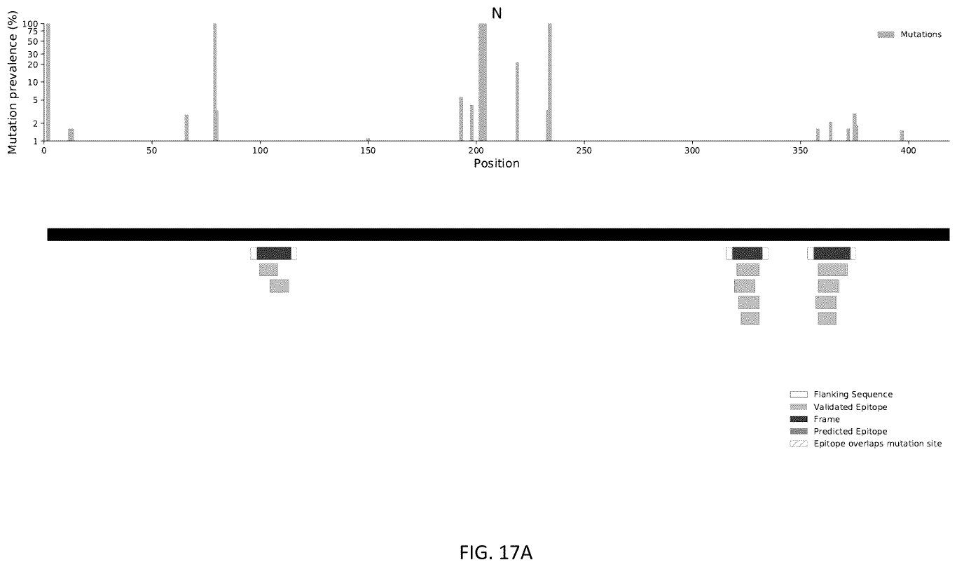

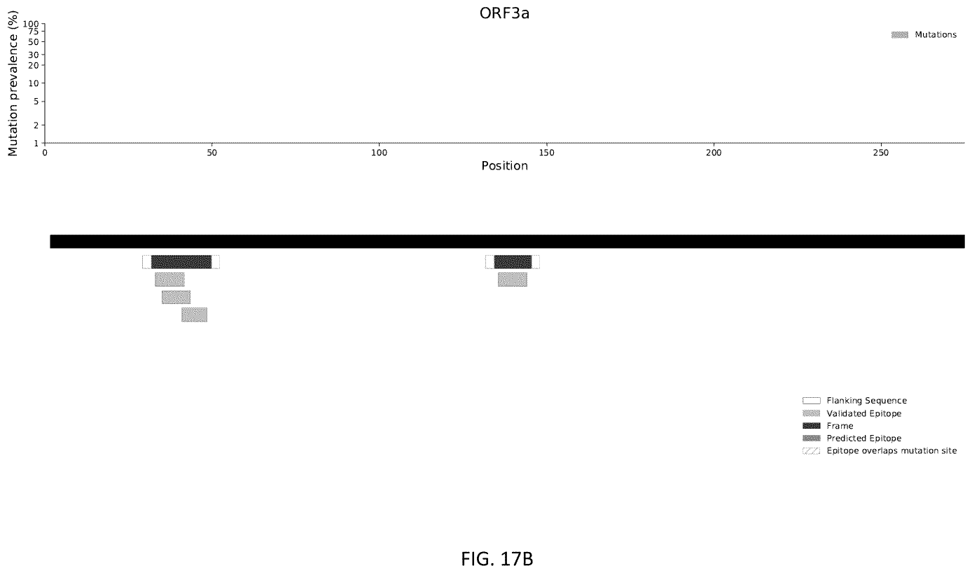

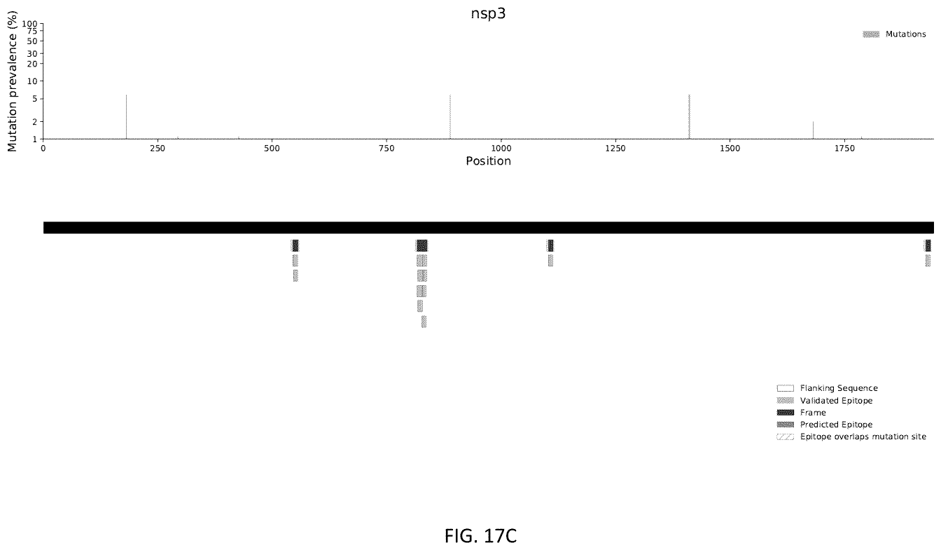

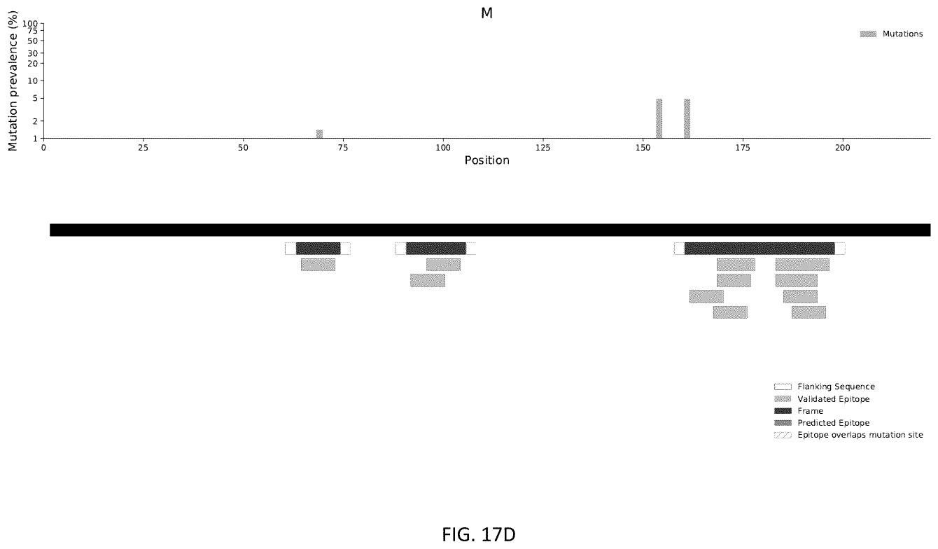

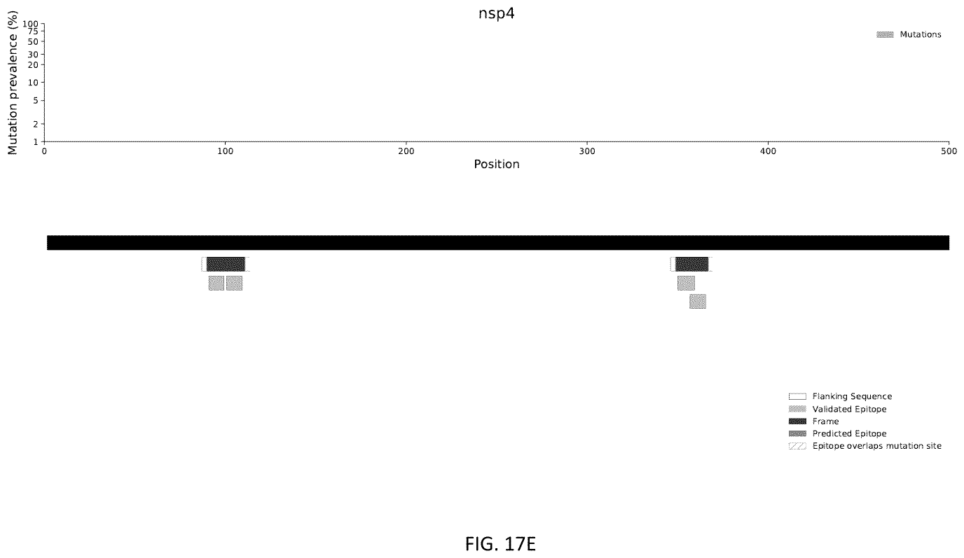

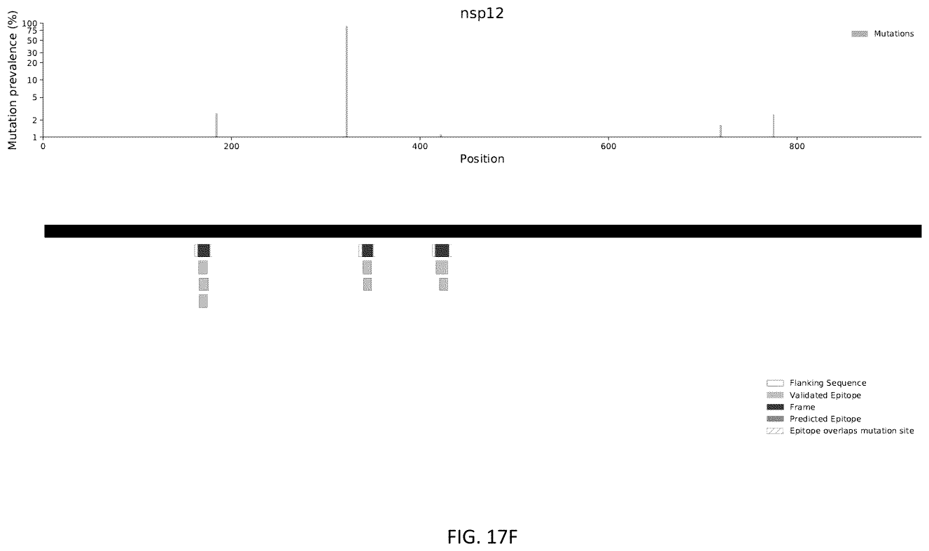

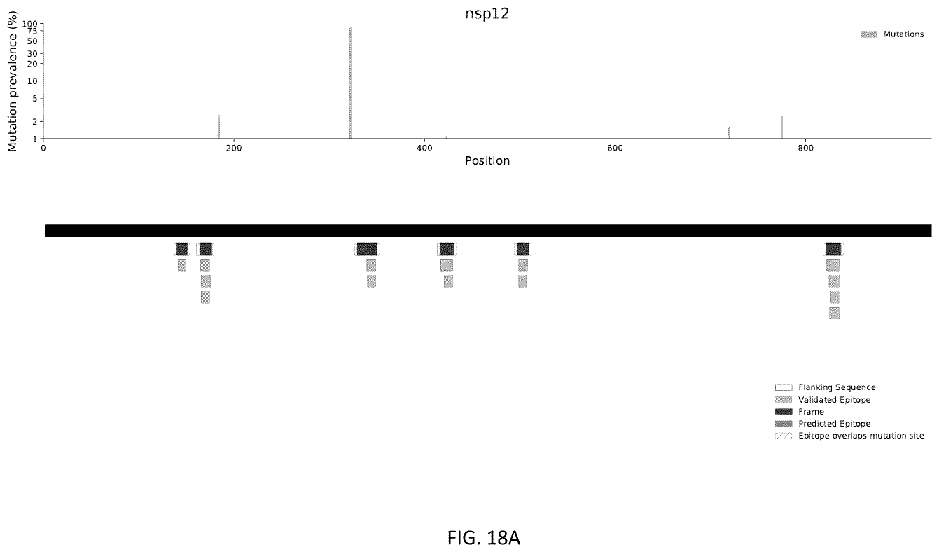

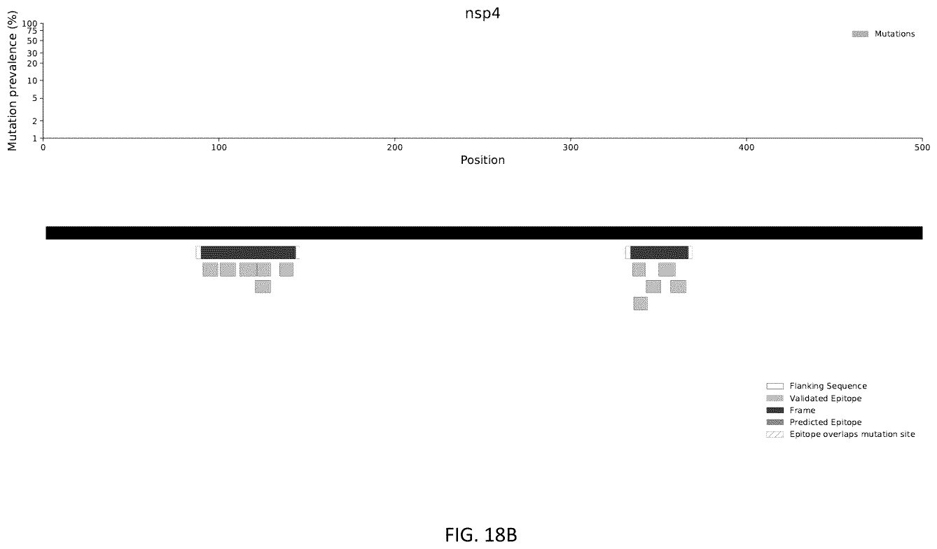

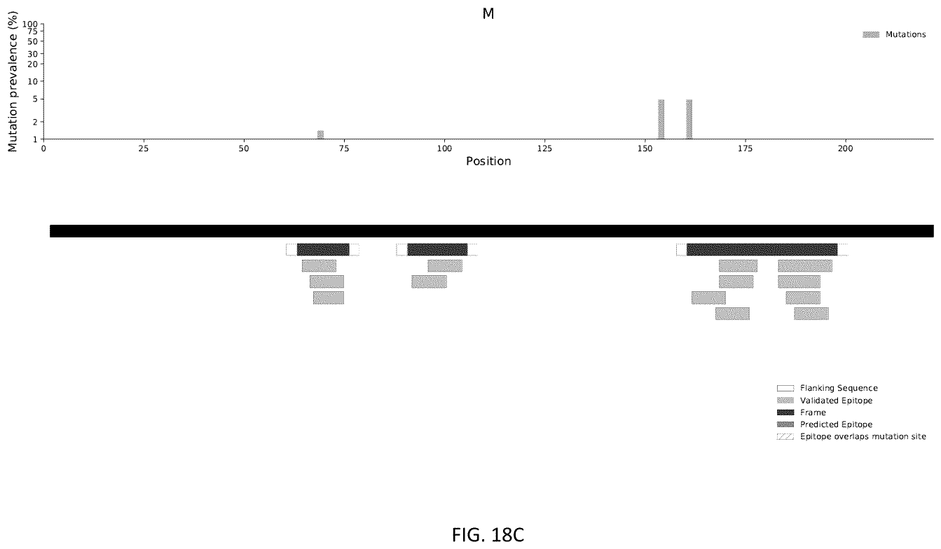

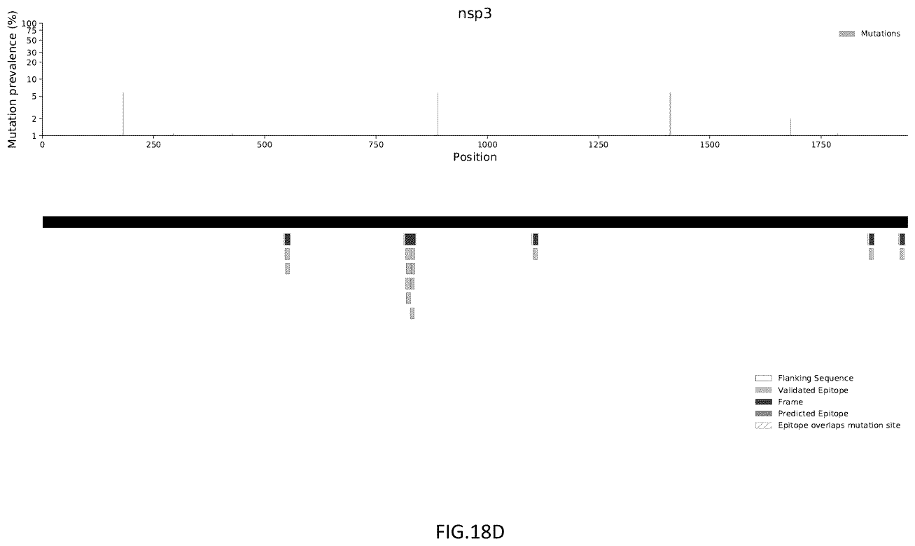

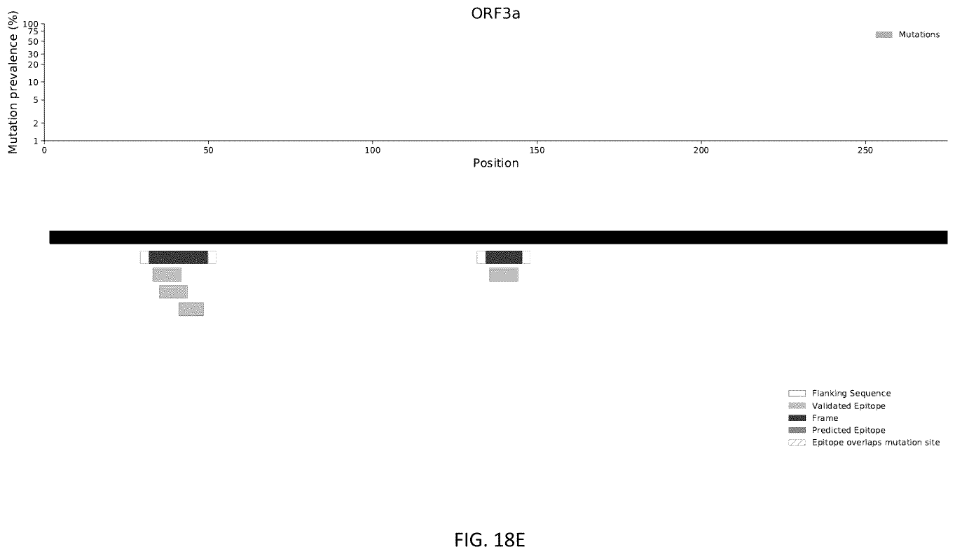

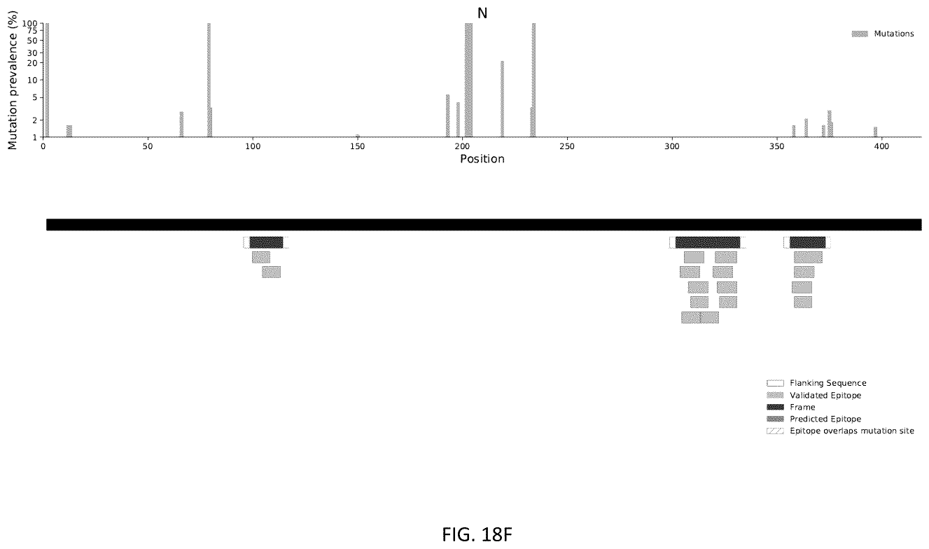

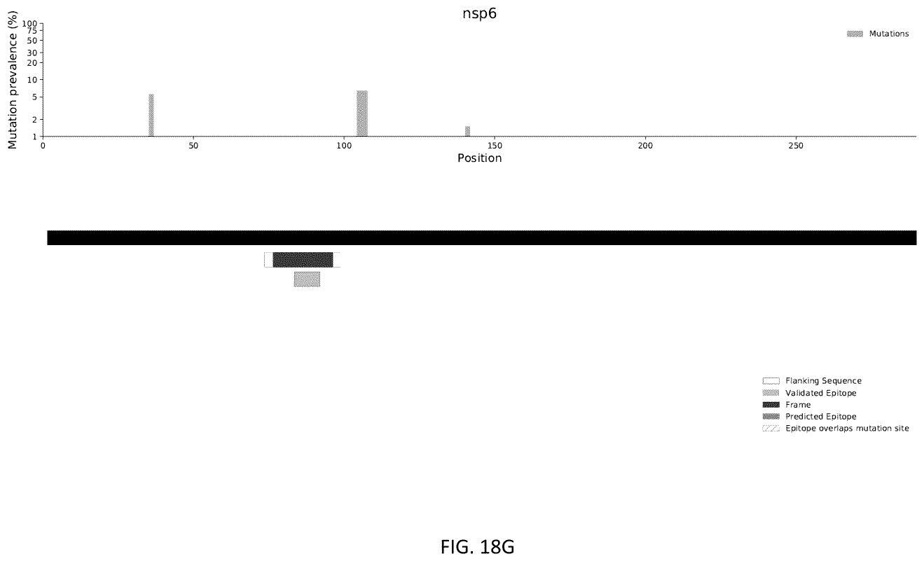

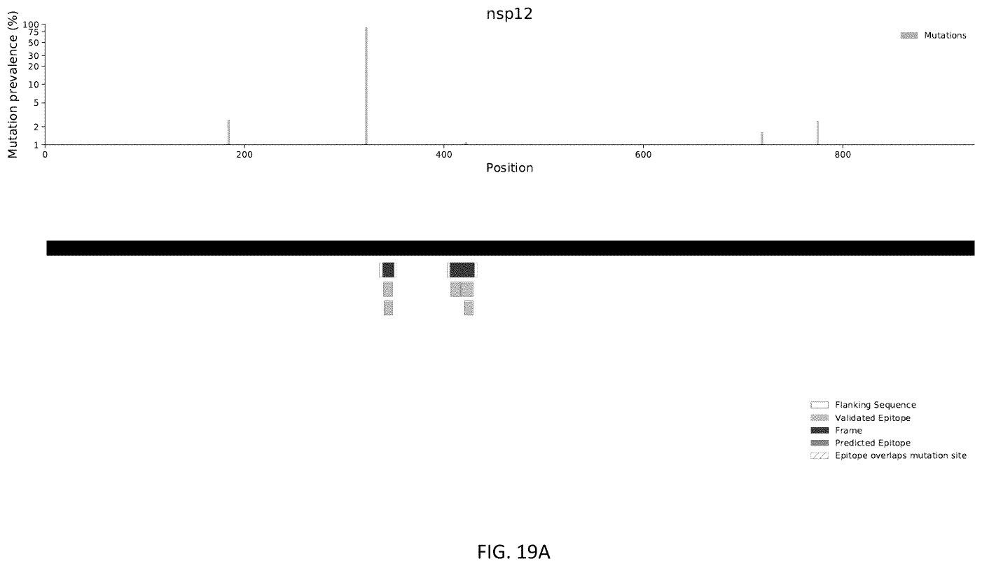

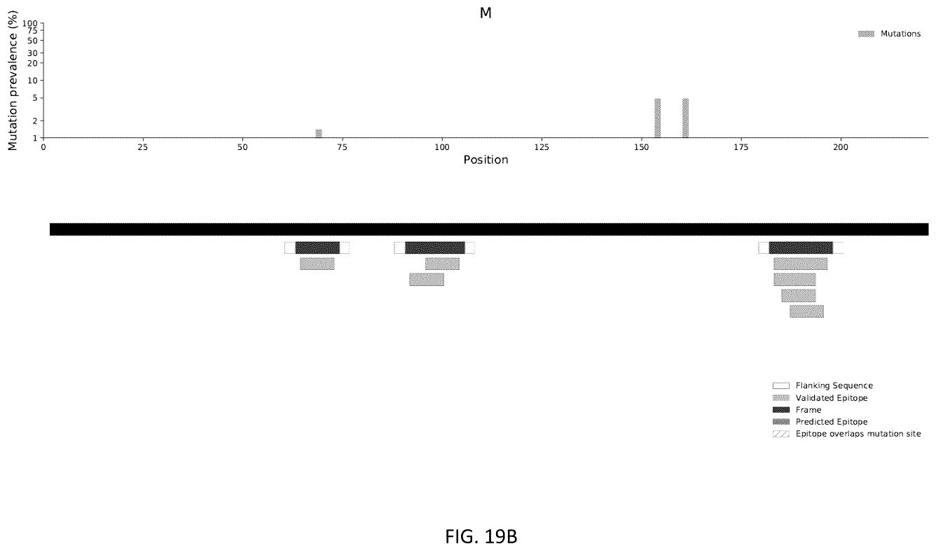

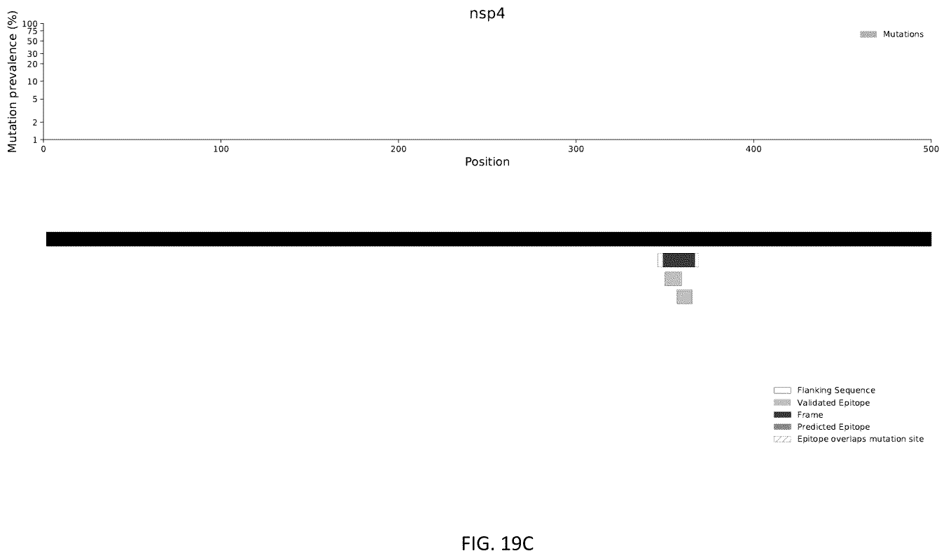

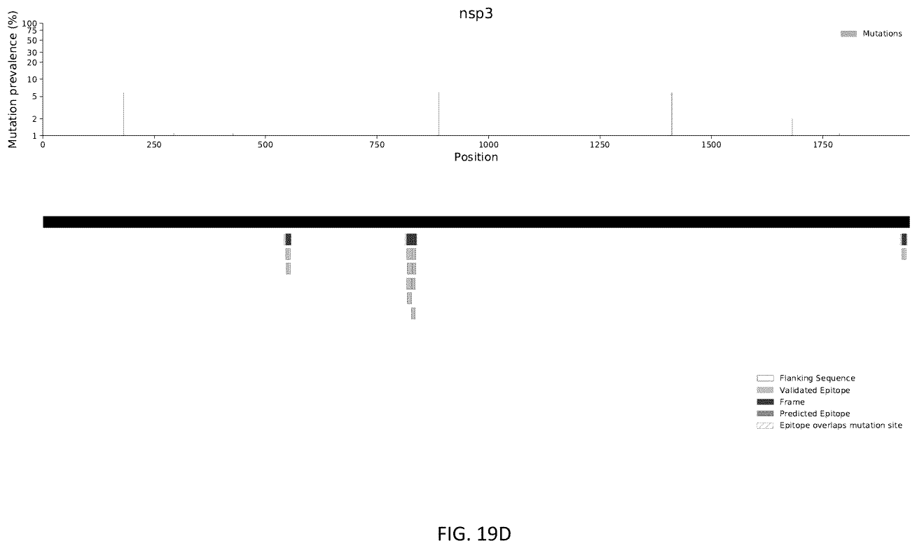

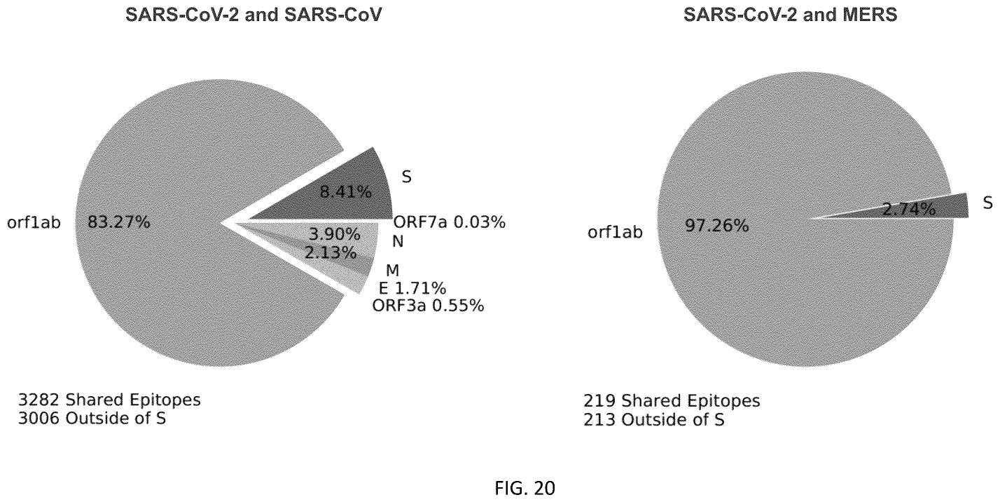

The following constructions were produced: (A) “TCE5”: Selected CD8+ epitopes predicted by the EDGE platform to be presented on MHC molecules for SARS-CoV-2 proteins other than Spike. The 15 selected epitopes are presented along with their order in the cassette in Table 7. The nucleotide sequence is shown in SEQ ID NO:91 and protein sequence shown in SEQ ID NO:92. FIG. 12 shows the estimated protection across the four indicated populations for TCE5. All populations are estimated to have coverage above 95% up to at least the threshold of 7 epitopes (last column); (B) “TCE10” starting with full Spike protein as the starting point and adding validated epitopes according to the above for a total size of 378 amino acids in addition to Spike (Table 9A, maps of epitopes covered in FIG. 17A-17F); (C) “TCE9” extended TCE10 and adding validated epitopes according to the above only if fully conserved between SARS and SARS-2 (e.g., as a pan-coronavirus vaccine), with certain frames extended (21 additional amino acids across all frames) to include additional predicted epitopes for alleles (i.e., not validated epitopes), for a total size of 556 amino acids in addition to Spike (Table 9B, maps of epitopes covered in FIG. 18A-18G); (C) “TCE11” starting with full Spike and Nucleocapsid proteins as the starting point and adding validated epitopes according to the above for a total size of 616 amino acids (197aa+full N) in addition to Spike (Table 9C, maps of epitopes covered in FIG. 19A-19D). Table 10A and Table 10B presents the magnitude coverage across various populations for each of TCE5, TCE9, TCE10, and TCE11 for SARS-CoV-2 and SARS/SARS-CoV-2 conserved epitopes, respectively. Notably, each of the vaccine constructs cover greater than 89% of each of the indicated populations with a validated response magnitude greater than 1000 and greater than 95% with a validated response magnitude greater than 100, while TCE9 covers greater than 74% of each of the indicated populations with a validated response magnitude greater than 1000 for epitopes conserved between SARS and SARS-2. FIG. 20 presents the percentages of shared candidate 9-mer epitope distribution between SARS-CoV-2 and SARS-CoV (left panel) and between SARS-CoV-2 and MERS (right panel), highlighting the significant number of conserved sequences outside of the Spike protein demonstrating the value of evaluating and including epitopes beyond those simply encoded by Spike, particularly with a goal of constructing a pan-coronavirus vaccine.

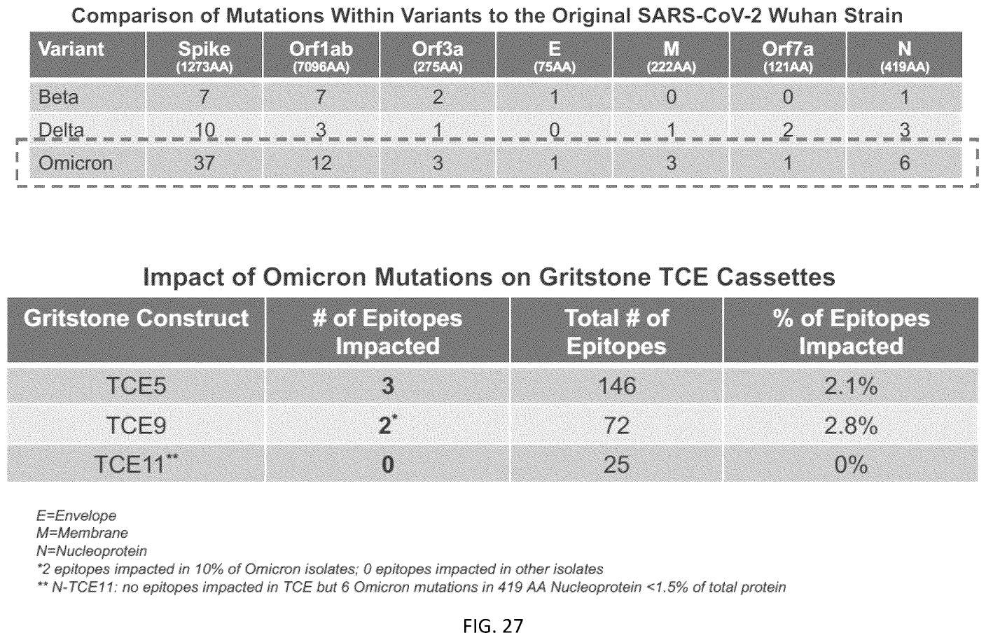

Omicron mutations were assessed for their impact on T-cell epitopes encoded by TCE5, TCE9, and TCE11. As shown in FIG. 27, Omicron mutations had minimal impact with 3, 2, and 0 epitopes impacted for TCE5, TCE9, and TCE11, respectively (representing 2.1%, 2.8%, and 0% of epitopes for each construct).

TABLE 8
Mutations > 1% frequency either overall or in specific strains

| Gene | Position | Strain | Frequency |
|---|---|---|---|
| M | 70 | b117 | 0.014 |
| M | 155 | N501Y_early | 0.048 |
| M | 162 | N501Y_early | 0.048 |
| N | 3 | b117 | 0.999 |
| N | 13 | N501Y_early | 0.016 |
| N | 14 | N501Y_early | 0.016 |
| N | 67 | | 0.028 |
| N | 80 | P1 | 1 |
| N | 81 | N501Y_early | 0.033 |
| N | 151 | | 0.011 |
| N | 194 | | 0.056 |
| N | 199 | | 0.041 |
| N | 203 | b117 | 1 |
| N | 204 | b117 | 1 |
| N | 205 | N501Y_early | 1 |
| N | 220 | | 0.218 |
| N | 234 | | 0.033 |
| N | 235 | b117 | 0.999 |
| N | 359 | N501Y_early | 0.016 |
| N | 365 | | 0.021 |
| N | 373 | N501Y_early | 0.016 |
| N | 376 | | 0.029 |
| N | 377 | | 0.018 |
| N | 398 | | 0.015 |
| ORF3a | 15 | b117 | 0.089 |
| ORF3a | 38 | | 0.011 |
| ORF3a | 57 | N501Y_early | 0.982 |
| ORF3a | 90 | b117 | 0.02 |
| ORF3a | 131 | N501Y_early | 0.036 |
| ORF3a | 171 | N501Y_early | 0.589 |
| ORF3a | 172 | | 0.039 |
| ORF3a | 202 | | 0.012 |
| ORF3a | 223 | | 0.016 |
| ORF3a | 251 | | 0.017 |
| ORF3a | 259 | N501Y_early | 0.018 |
| S | 5 | | 0.012 |
| S | 18 | N501Y_early | 0.381 |
| S | 54 | N501Y_early | 0.016 |
| S | 69 | b117 | 1 |
| S | 70 | b117 | 1 |
| S | 80 | N501Y_early | 1 |
| S | 98 | | 0.012 |
| S | 144 | b117 | 1 |
| S | 215 | N501Y_early | 1 |
| S | 222 | | 0.198 |
| S | 241 | N501Y_early | 1 |
| S | 242 | N501Y_early | 1 |
| S | 243 | N501Y_early | 1 |
| S | 246 | N501Y_early | 0.016 |
| S | 262 | | 0.011 |
| S | 316 | N501Y_early | 0.016 |
| S | 384 | N501Y_early | 0.016 |
| S | 417 | N501Y_early | 0.984 |
| S | 439 | | 0.017 |
| S | 477 | | 0.048 |
| S | 484 | N501Y_early | 1 |
| S | 501 | b117 | 1 |
| S | 570 | b117 | 1 |
| S | 614 | b117 | 1 |
| S | 681 | b117 | 1 |
| S | 682 | cleavage | 1 |
| S | 683 | cleavage | 1 |
| S | 685 | cleavage | 1 |
| S | 688 | N501Y_early | 0.016 |
| S | 701 | N501Y_early | 1 |
| S | 706 | b117 | 0.018 |
| S | 716 | b117 | 1 |
| S | 982 | b117 | 1 |
| S | 1078 | N501Y_early | 0.016 |
| S | 1117 | N501Y_early | 0.016 |
| S | 1118 | b117 | 1 |
| S | 1150 | N501Y_early | 0.016 |
| nsp12 | 4577 | | 0.026 |
| nsp12 | 4646 | | 0.01 |
| nsp12 | 4715 | | 0.885 |
| nsp12 | 4815 | | 0.011 |
| nsp12 | 5112 | | 0.016 |
| nsp12 | 5168 | | 0.025 |
| nsp13 | 5542 | | 0.025 |
| nsp13 | 5585 | | 0.026 |
| nsp13 | 5614 | | 0.022 |
| nsp13 | 5784 | | 0.027 |
| nsp13 | 5922 | | 0.017 |
| nsp14 | 6054 | | 0.024 |
| nsp14 | 6426 | | 0.011 |
| nsp15 | 6485 | | 0.015 |
| nsp16 | 7014 | | 0.028 |
| nsp2 | 265 | | 0.132 |
| nsp2 | 300 | | 0.042 |
| nsp3 | 1001 | | 0.059 |
| nsp3 | 1113 | | 0.011 |
| nsp3 | 1246 | | 0.011 |
| nsp3 | 1361 | | 0.01 |
| nsp3 | 1708 | | 0.058 |
| nsp3 | 2230 | | 0.058 |
| nsp3 | 2501 | | 0.02 |
| nsp3 | 2606 | | 0.011 |
| nsp4 | 3087 | | 0.025 |
| nsp5 | 3278 | | 0.016 |
| nsp5 | 3352 | | 0.034 |
| nsp5 | 3353 | | 0.013 |
| nsp5 | 3371 | | 0.013 |
| nsp6 | 3606 | | 0.056 |
| nsp6 | 3675 | | 0.064 |
| nsp6 | 3676 | | 0.064 |
| nsp6 | 3677 | | 0.064 |
| nsp6 | 3711 | | 0.015 |
| nsp7 | 3884 | | 0.01 |
| nsp9 | 4241 | | 0.016 |

TABLE 9A
TCE10 Cassette (Order of Frames as Shown)

| | Frame Start | Frame End | | SEQ |
|---|---|---|---|---|
| Gene | in Gene | in Gene | Frame sequence | ID NO: |
| N | 356 | 378 | HIDAYKTFPPTEPKKDKKKKADE | 27944 |
| ORF3a | 133 | 149 | CRSKNPLLYDANYFLCW | 27945 |
| nsp3 | 1632 | 1660 | FEYYHTTDPSFLGRYMSALNHTKKWKYPQ | 27946 |
| nsp3 | 1360 | 1377 | ISNEKQEILGTVSWNLRE | 27947 |
| M | 160 | 203 | DIKDLPKEITVATSRTLSYYKLGASQ | 27948 |
| | | | RVAGDSGFAAYSRYRIGN | |
| ORF3a | 29 | 52 | VRATATIPIQASLPFGWLIVGVAL | 27949 |
| nsp4 | 3111 | 3134 | YLTFYLTNDVSFLAHIQWMVMFTP | 27950 |
| M | 89 | 109 | GLMWLSYFIASFRLFARTRSM | 27951 |
| nsp12 | 4553 | 4571 | DWYDFVENPDILRVYANLG | 27952 |
| nsp3 | 1919 | 1935 | PYPNASFDNFKFVCDNI | 27953 |
| nsp12 | 4806 | 4826 | NFNKDFYDFAVSKGFFKEGSS | 27954 |
| nsp12 | 4728 | 4745 | DGVPFVVSTGYHFRELGV | 27955 |
| nsp4 | 2850 | 2876 | ACPLIAAVITREVGFVVPGLPGTILRT | 27956 |
| nsp3 | 2745 | 2761 | ATTRQVVNVVTTKIALK | 27957 |
| N | 318 | 337 | SRIGMEVTPSGTWLTYTGAI | 27958 |
| M | 61 | 77 | TLACFVLAAVYRINWIT | 27959 |
| N | 96 | 117 | GGDGKMKDLSPRWYFYYLGTGP | 27960 |

TABLE 9B
TCE9 Cassette (Order of Frames as Shown)

| | Frame Start | Frame End | | SEQ |
|---|---|---|---|---|
| Gene | in Gene | in Gene | Frame sequence | ID NO: |
| nsp12 | 4719 | 4745 | GPLVRKIFVDGVPFVVSTGYHFRELGV | 27961 |
| nsp4 | 3096 | 3134 | PVYSFLPGVYSVIYLYLTFYLTNDVSF | 27962 |
| | | | LAHIQWMVMFTP | |
| M | 89 | 109 | GLMWLSYFIASFRLFARTRSM | 27951 |
| nsp12 | 4888 | 4905 | NNLDKSAGFPFNKWGKAR | 27963 |
| nsp3 | 2745 | 2761 | ATTRQVVNVVTTKIALK | 27957 |
| M | 61 | 79 | TLACFVLAAVYRINWITGG | 27964 |
| nsp3 | 1919 | 1935 | PYPNASFDNFKFVCDNI | 27953 |
| nsp3 | 2676 | 2692 | TYNKVENMTPRDLGACI | 27965 |
| ORF3a | 29 | 52 | VRATATIPIQASLPFGWLIVGVAL | 27949 |
| nsp12 | 4806 | 4826 | NFNKDFYDFAVSKGFFKEGSS | 27954 |
| N | 96 | 117 | GGDGKMKDLSPRWYFYYLGTGP | 27960 |
| nsp12 | 5213 | 5234 | KQGDDYVYLPYPDPSRILGAGC | 27966 |
| N | 356 | 378 | HIDAYKTFPPTEPKKDKKKKADE | 27944 |
| ORF3a | 133 | 149 | CRSKNPLLYDANYFLCW | 27945 |
| nsp6 | 3643 | 3668 | SLATVAYFNMVYMPASWVMRIMTWLD | 27967 |
| nsp12 | 4529 | 4545 | GNCDTLKEILVTYNCCD | 27968 |
| nsp3 | 1630 | 1660 | EAFEYYHTTDPSFLGRYMSALNHTKKWKYPQ | 27969 |
| nsp3 | 1360 | 1377 | ISNEKQEILGTVSWNLRE | 27947 |
| M | 160 | 203 | DIKDLPKEITVATSRTLSYYKLGASQRVAGD | 27948 |
| | | | SGFAAYSRYRIGN | |
| N | 301 | 337 | WPQIAQFAPSASAFFGMSRIGMEVTPSGT | 27970 |
| | | | WLTYTGAI | |
| nsp12 | 4553 | 4571 | DWYDFVENPDILRVYANLG | 27952 |
| nsp4 | 2850 | 2909 | ACPLIAAVITREVGFVVPGLPGTILRTTNG | 27971 |
| | | | DFLHFLPRVFSAVGNICYTPSKLIEYTDFA | |

TABLE 9C
TCE11 Cassette (Order of Frames as Shown)

| | Frame | Frame | | SEQ |
|---|---|---|---|---|
| | Start | End | | ID |
| Gene | in Gene | in Gene | Frame sequence | NO: |
| nsp12 | 4896 | 4826 | NVAFQTVKPGNFNKDFYDF | 27972 |
| | | | AVSKGFFKEGSS | |
| M | 182 | 203 | GASQRVAGDSGFAAYSRYR | 27973 |
| | | | IGN | |
| nsp12 | 4728 | 4745 | DGVPFVVSTGYHFRELGV | 27955 |
| nsp4 | 3111 | 3134 | YLTFYLTNDVSFLAHIQWM | 27950 |
| | | | VMFTP | |
| M | 89 | 109 | GLMWLSYFIASFRLFART | 27951 |
| | | | RSM | |
| nsp3 | 1632 | 1660 | FEYYHTTDPSFLGRYMSA | 27946 |
| | | | LNHTKKWKYPQ | |
| nsp3 | 1360 | 1377 | ISNEKQEILGTVSWNLRE | 27947 |
| nsp3 | 2745 | 2761 | ATTRQVVNVVTTKIALK | 27957 |
| M | 61 | 77 | TLACFVLAAVYRINWIT | 27959 |

TABLE 10A
Population Coverages for SARS-CoV-2 Validated
Epitopes (Excluding mutations >5%)

| Cassette | Magnitude | Starting Protein(s) | AFA | API | EUR | HIS | Mean | Min |
|---|---|---|---|---|---|---|---|---|
| Spike | 1 | S | 0.95 | 0.89 | 1 | 0.98 | 0.95 | 0.89 |
| S + N + TCE11 | 1 | S, N | 0.97 | 0.95 | 1 | 0.99 | 0.98 | 0.95 |
| S + TCE10 | 1 | S | 0.97 | 0.95 | 1 | 0.99 | 0.98 | 0.95 |
| S + TCE9 | 1 | S | 0.97 | 0.95 | 1 | 0.99 | 0.98 | 0.95 |
| S + TCE5 | 1 | S | 0.95 | 0.95 | 1 | 0.98 | 0.97 | 0.95 |
| Spike | 100 | S | 0.92 | 0.86 | 1 | 0.96 | 0.93 | 0.86 |
| S + N + TCE11 | 100 | S, N | 0.95 | 0.95 | 1 | 0.99 | 0.97 | 0.95 |
| S + TCE10 | 100 | S | 0.95 | 0.95 | 1 | 0.99 | 0.97 | 0.95 |
| S + TCE9 | 100 | S | 0.95 | 0.95 | 1 | 0.99 | 0.97 | 0.95 |
| S + TCE5 | 100 | S | 0.92 | 0.94 | 1 | 0.97 | 0.96 | 0.92 |
| Spike | 1000 | S | 0.6 | 0.62 | 0.91 | 0.75 | 0.72 | 0.6 |
| S + N + TCE11 | 1000 | S, N | 0.89 | 0.92 | 1 | 0.96 | 0.94 | 0.89 |
| S + TCE10 | 1000 | S | 0.9 | 0.92 | 1 | 0.97 | 0.95 | 0.9 |
| S + TCE9 | 1000 | S | 0.9 | 0.92 | 1 | 0.97 | 0.95 | 0.9 |
| S + TCE5 | 1000 | S | 0.68 | 0.7 | 0.93 | 0.8 | 0.78 | 0.68 |
| Spike | 2000 | S | 0.39 | 0.49 | 0.73 | 0.56 | 0.54 | 0.39 |
| S + N + TCE11 | 2000 | S, N | 0.66 | 0.72 | 0.96 | 0.82 | 0.79 | 0.66 |
| S + TCE10 | 2000 | S | 0.72 | 0.73 | 0.96 | 0.85 | 0.81 | 0.72 |
| S + TCE9 | 2000 | S | 0.76 | 0.77 | 0.98 | 0.9 | 0.85 | 0.76 |
| S + TCE5 | 2000 | S | 0.49 | 0.54 | 0.81 | 0.66 | 0.62 | 0.49 |

TABLE 10B
Population Coverages for Validated Conserved Epitopes
between SARS and SARS-CoV-2 (Excluding mutations >5%)

| Cassette | Magnitude | Starting Protein(s) | AFA | API | EUR | HIS | Mean | Min |
|---|---|---|---|---|---|---|---|---|
| Spike | 1 | S | 0.74 | 0.82 | 0.98 | 0.91 | 0.87 | 0.74 |
| S + N + TCE11 | 1 | S, N | 0.89 | 0.93 | 1 | 0.97 | 0.95 | 0.89 |
| S + TCE10 | 1 | S | 0.94 | 0.93 | 1 | 0.97 | 0.96 | 0.93 |
| S + TCE9 | 1 | S | 0.94 | 0.93 | 0.99 | 0.97 | 0.96 | 0.93 |
| S + TCE5 | 1 | S | 0.87 | 0.91 | 0.99 | 0.95 | 0.93 | 0.87 |
| Spike | 100 | S | 0.74 | 0.78 | 0.98 | 0.89 | 0.85 | 0.74 |
| S + N + TCE11 | 100 | S, N | 0.89 | 0.91 | 0.99 | 0.95 | 0.94 | 0.89 |
| S + TCE10 | 100 | S | 0.94 | 0.93 | 0.99 | 0.97 | 0.96 | 0.93 |
| S + TCE9 | 100 | S | 0.94 | 0.92 | 0.99 | 0.97 | 0.96 | 0.92 |
| S + TCE5 | 100 | S | 0.86 | 0.88 | 0.98 | 0.93 | 0.91 | 0.86 |
| Spike | 1000 | S | 0.18 | 0.2 | 0.46 | 0.27 | 0.28 | 0.18 |
| S + N + TCE11 | 1000 | S, N | 0.49 | 0.67 | 0.76 | 0.62 | 0.63 | 0.49 |
| S + TCE10 | 1000 | S | 0.52 | 0.56 | 0.71 | 0.55 | 0.59 | 0.52 |
| S + TCE9 | 1000 | S | 0.74 | 0.81 | 0.92 | 0.84 | 0.83 | 0.74 |
| S + TCE5 | 1000 | S | 0.33 | 0.42 | 0.62 | 0.49 | 0.47 | 0.33 |
| Spike | 2000 | S | 0.01 | 0.03 | 0.08 | 0.03 | 0.04 | 0.01 |
| S + N + TCE11 | 2000 | S, N | 0.24 | 0.2 | 0.41 | 0.28 | 0.28 | 0.2 |
| S + TCE10 | 2000 | S | 0.2 | 0.15 | 0.34 | 0.17 | 0.21 | 0.15 |
| S + TCE9 | 2000 | S | 0.4 | 0.37 | 0.7 | 0.51 | 0.49 | 0.37 |
| S + TCE5 | 2000 | S | 0.21 | 0.11 | 0.34 | 0.24 | 0.22 | 0.11 |

### XIV.I. SARS-CoV-2 Prime-Boost Regimens Featuring Spike Proteins from Different SARS-CoV-2 Subtype Isolates Produces Responses to Spike in Non-Human Primates

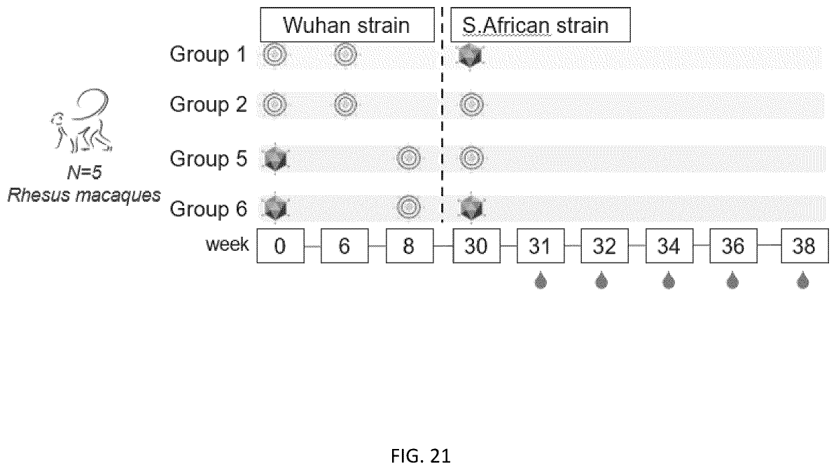

ChAd and SAM vaccine platforms encoding different subtype isolates of the SARS-CoV-2 Spike protein were assessed in Indian rhesus macaques as part of homologous or heterologous prime/boost regimens, as shown in FIG. 21 and presented in Table 11.

TABLE 11
NHP Study Design for SARS-COV-2 Isolate Vaccine Regimens

| | Prime | Boost 1 | Boost 2 |
|---|---|---|---|
| Group # | (week 0) | (week 6 or 8) | (week 30) |
| 1 | SAM-S_{D614G}; | SAM-S_{D614G}; | ChAd-S_{B1351}-TCE5; |
| | 300 ug | 300 ug | 5e11 vp |
| 2 | SAM-S_{D614G}; | SAM-S_{D614G}; | SAM-TCE5-S_{B13519}; |
| | 30 ug | 30 ug | 30 ug |
| 5 | ChAd-S_{D614G}; | SAM-S_{D614G}; | SAM-TCE5-S_{B1351}; |
| | 1e12 vp | 100 ug | 30 ug |
| 6 | ChAd.S_{D614G}; | SAM-S_{D614G}; | ChAd-S_{B1351}-TCE5; |
| | 5e10 vp | 100 ug | 5e11 vp |

Prime: ChAdV - “CTSpike_{g}” (SEQ ID NO: 79); SAM - “IDTSpike_{g}” (SEQ ID NO: 69)
Boost 1: SAM - “IDTSpike_{g}” (SEQ ID NO: 69)
Boost 2: all CT-F2P versions of Spike variant B.1.351; TCE5 see Table 7

NHPs were first immunized with a priming dose of either a ChAd platform including a Spike-encoding cassette featuring “ChAd-S_{D614G}; CT” (SEQ ID NO:79) or a SAM platform including a Spike-encoding cassette featuring “SAM-S_{D614G}; IDT” (SEQ ID NO:69) at the indicated doses. NHPs were then administered a first boost at weeks 6 or 8 with the SAM platform including a Spike-encoding cassette featuring “SAM-S_{D614G}; IDT” at the indicated doses. NHPs were then administered a second boost at week 30 with either a ChAd platform including a B.1.351 Spike variant-encoding cassette featuring Cool Tool sequence optimization (“CT”) and the F2P modification described herein (“F2P”) [SEQ ID NO:112] or a SAM platform including the same B.1.351 Spike variant (each platform also included the TCE5 T cell epitope cassette, see Table 7, in the orientation shown). The ChAdV antigen cassette is shown in SEQ ID NO:113. NHPs were monitored over time, as described herein.

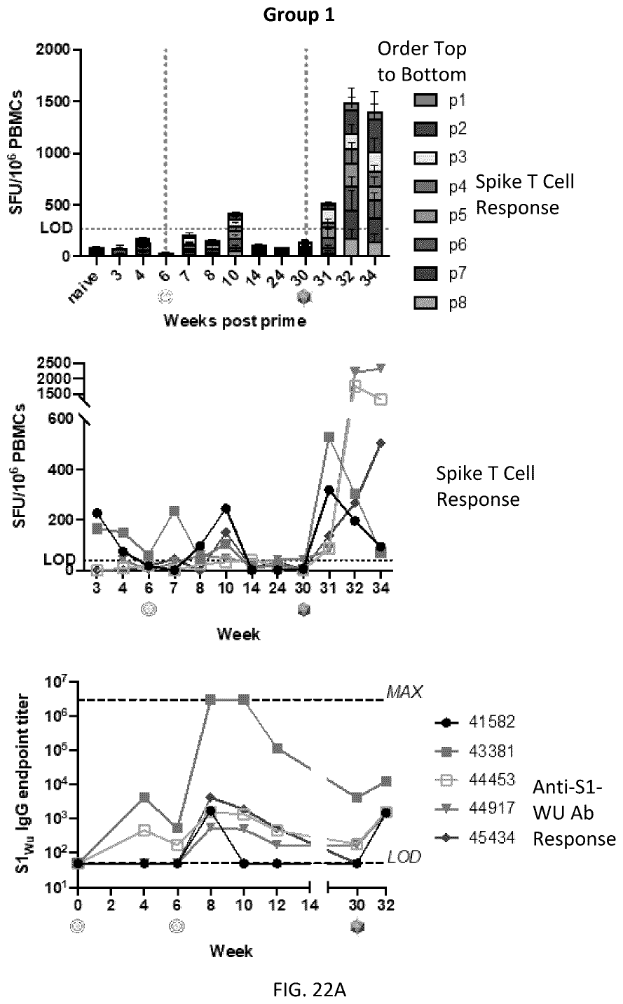

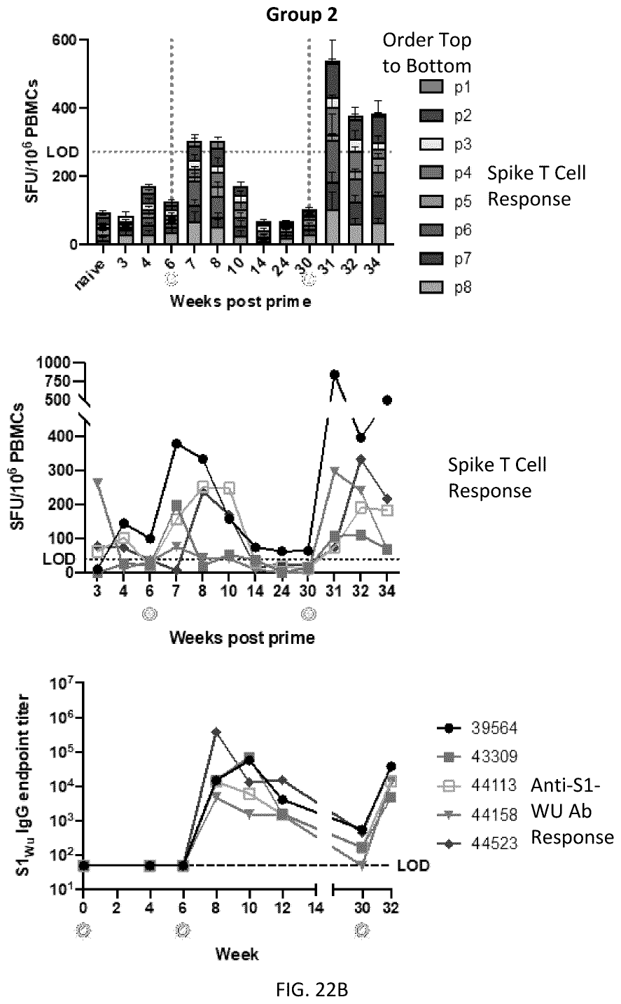

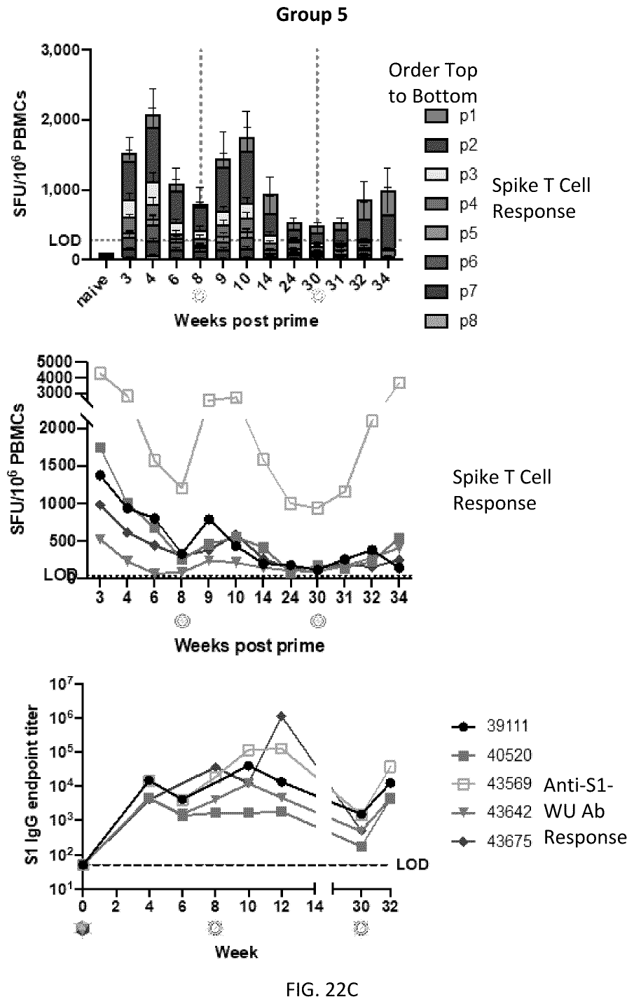

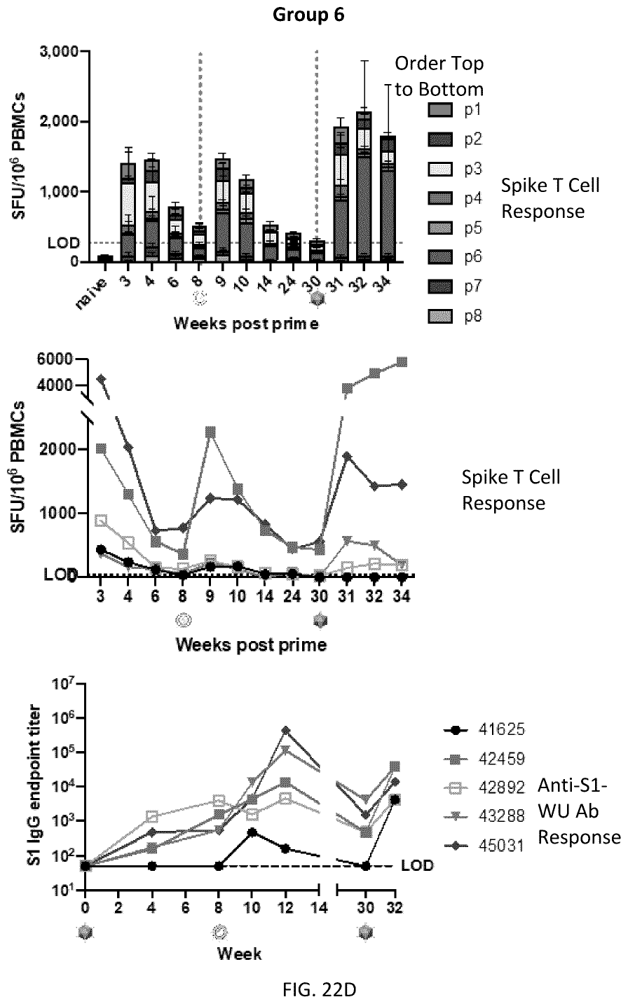

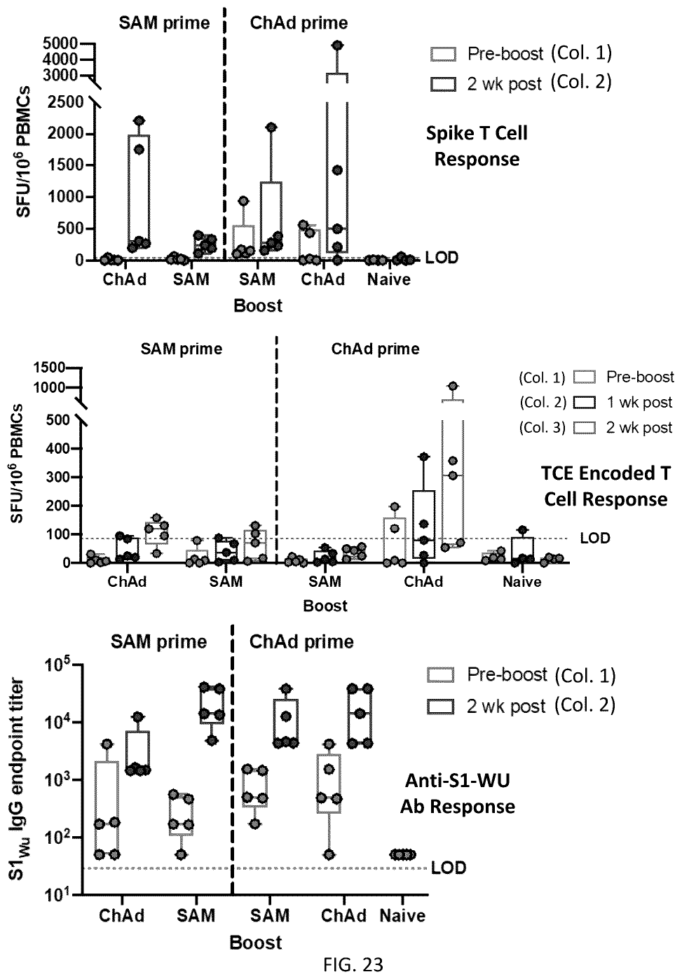

As shown in FIGS. 22A, 22B, 22C, and 22D, the various vaccine regimens (Groups 1, 2, 5, and 6, respectively) produced T cell responses across multiple Spike T cell epitope pools (top panels). T cell responses for individual NHPs directed to a single large Spike T cell epitope pool was heterogenous (middle panels and summarized in FIG. 23 top panel), with each boost generally producing an increased T cell response, including production of a robust response in some (e.g., two NHPs in Group 1 following Boost 2). Spike-specific IgG antibody titers were detected and increased following each boost (bottom panels and summarized in FIG. 23 bottom panel) in all five NHP animals assessed. T cell responses to the TCE5-encoded epitopes, though generally small, trended upwards following Boost 2 (the first administration of a vaccine including TCE5), with generally stronger responses with administration of the ChAdV platform vaccine (FIG. 23 middle panel). Accordingly, the data demonstrate a vaccine regimen including a boost with a Spike variant encoding vaccine produced T cell and antibody responses.

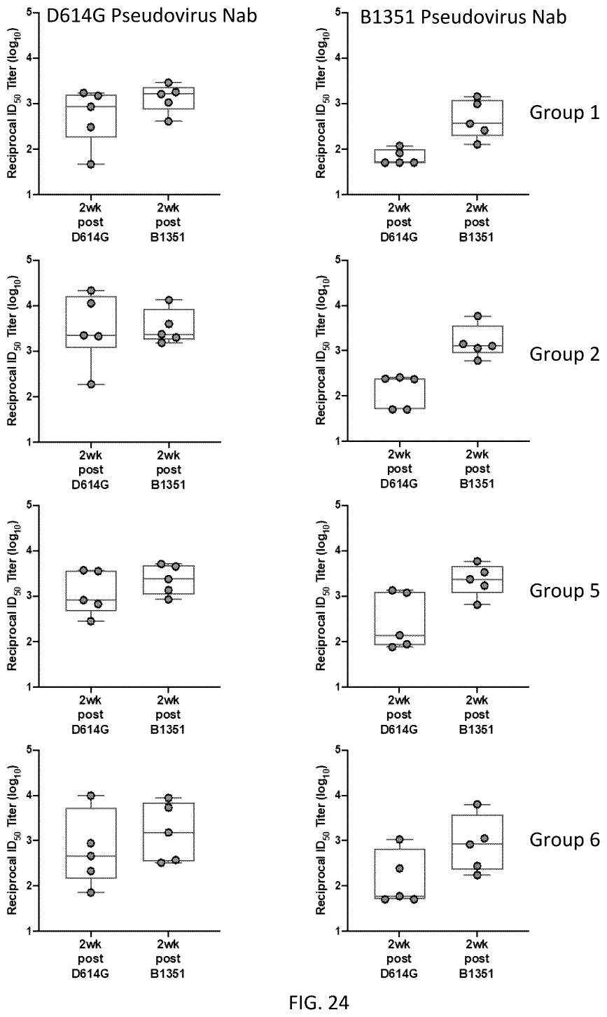

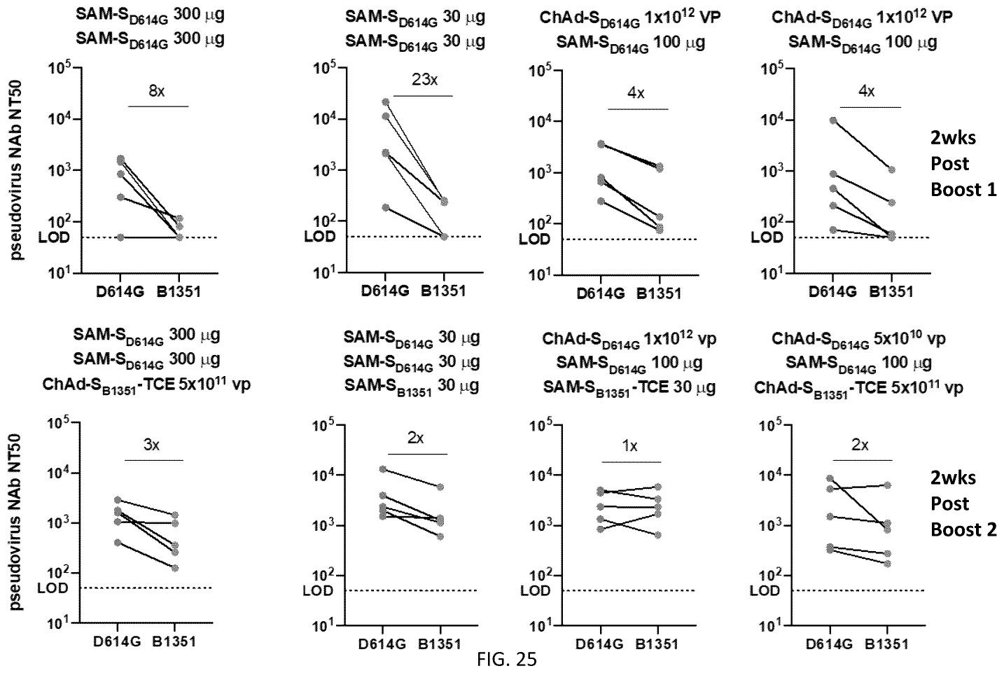

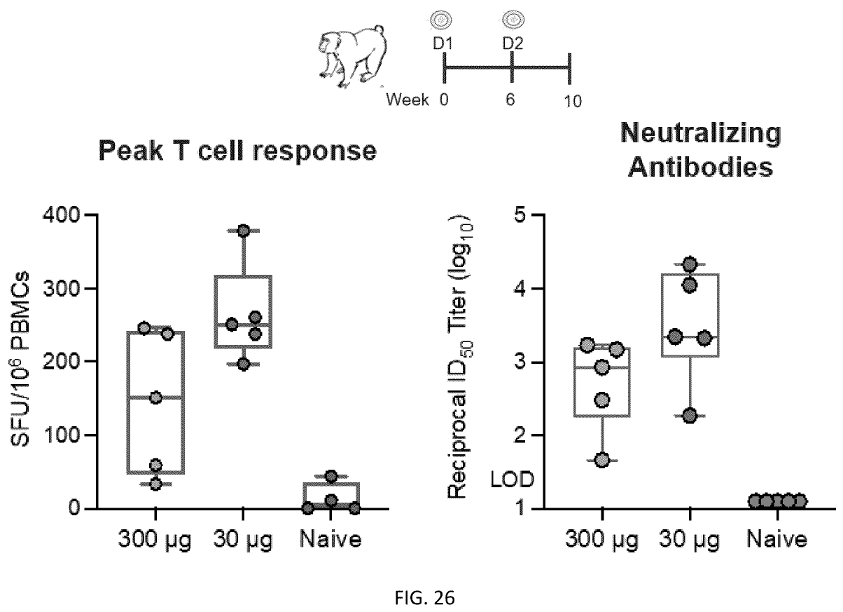

Antibody responses were further assessed for neutralizing antibody production to both the D614G pseudovirus and B.1.351 pseudovirus. As shown in FIG. 24, neutralizing antibody (Nab) titers against the D614G pseudovirus were detected following Boost 1 across the four groups, with Nab titers generally the same following Boost 2 (left panels). Following Boost 1, cross-neutralizing antibody titers against the B.1.351 pseudovirus, while detected, were distinctly lower than the Nab titer against the D614G pseudovirus (right panel, column 1). However, following administration of Boost 2 encoding the B.1.351 spike variant, Nab titers against the B.1.351 pseudovirus were noticeably increased (right panels, column 2), notably with similar Nab titer levels to that against the D614G pseudovirus following Boost 1. These results are further demonstrated in FIG. 25, comparing the relative Nab titer levels against each of the pseudoviruses illustrating the reduced cross-neutralizing capacity against the B.1.351 pseudovirus following Boost 1 (top panels) and rescuing of the same following Boost 2 (bottom panels) across each of the vaccine regimens assessed. FIG. 26 shows T cell responses (left panel) and Nab titer levels (right panel) for Rhesus macaques immunized twice with SAM encoding SARS-CoV-2 Spike antigen at a specified dose of either 30 μg or 300 μg, with 30 μg doses producing a more robust response.

The data demonstrate the various vaccine regimens produced both T cell and antibody responses against the encoded antigens in NHPs, notably demonstrating subsequent immunization with Spike variant encoding vaccines noticeably improved Nab titers against the respective variant pseudovirus.

### XIV.J. Omicron SARS-CoV-2 Vaccine Assessment

A phase 1, open-label, dose escalation, non-randomized study of homologous and heterologous prime-boost vaccination schedules to examine safety, tolerability, and immunogenicity of investigational Chimpanzee Adenovirus serotype 68 (ChAd) and/or self-amplifying mRNA (SAM) vectors expressing either Severe Acute Respiratory Syndrome Coronavirus 2 (SARS-CoV-2) spike alone, or spike plus additional SARS-CoV-2 T cell epitopes (TCE), such as epitopes presented in Tables A-F or T cell epitope cassettes described in Tables 7, 9A, 9B, or 9C, in healthy adult subjects is conducted. Assessment includes vaccine strategies including homologous SAM prime/SAM boost, homologous ChAd prime/ChAd boost and heterologous ChAd prime/SAM boost combinations.

The primary objective of this study is to assess the safety and tolerability of different doses of SAM-Nuc-TCE11-SpikeB.1.1.529 sequence (SEQ ID NO: 27976) when administered as prime and/or boost in healthy adult subjects including older adult subjects, e.g., administered in (i) a homologous SAM prime/boost vaccine regimen; (ii) following prior vaccination with a ChAdV68-based vaccine platform described herein; or (iii) following prior vaccination with a commercially available SARS-CoV-2 vaccine platform, including, but not limited to, Comirnaty® (BioNTech/Pfizer), mRNA-1273/SpikeVax® (Moderna), AZD1222/Covishield® (Oxford/AstraZeneca), or Ad26.COV2.S/JNJ-78436735 (Janssen/Johnson & Johnson).

ChAdV68 “Chimpanzee Adenovirus serotype 68”: a replication-defective, E1, E3 E4Orf2-4 deleted adenoviral vector based on chimpanzee adenovirus 68 (C68, 68/SAdV-25, originally designated as Pan 9), which belongs to the sub-group E adenovirus family. A single 0.5 mL or 1.0 mL intramuscular injection (depending on dose level) is administered in the deltoid muscle. When possible, the prime vaccine and boost vaccine is administered in different arms. ChAdV68 is administered at doses of 5×10\hat{ }10 or 1×10\hat{ }11 viral particles (or adjusted as determined during the study).

SAM-LNP “Self-Amplifying mRNA-Lipid Nanoparticles”: a SAM vector based on Venezuelan Equine Encephalitis Virus (VEEV). A single 0.5 mL intramuscular injection is administered in the deltoid muscle. When possible, the prime vaccine and boost vaccine is administered in different arms. The specific SAM construct assessed is SAM-Nuc-TCE11-SpikeB.1.1.529 sequence (SEQ ID NO: 27976) and is administered at doses of 1 mcg, 3 mcg, 10 mcg, 30 mcg, or 100 mcg (or adjusted as determined during the study).

The diluent used for this study is 0.9% Sodium Chloride Injection, USP, and is a sterile, nonpyrogenic, isotonic solution of sodium chloride and water for injection. Each milliliter (mL) contains sodium chloride 9 mg. It contains no bacteriostat, antimicrobial agent or added buffer and is supplied only in single-dose containers to dilute or dissolve drugs for injection. 0.308 mOsmol/mL (calc.). 0.9% Sodium Chloride Injection, USP contains no preservatives.

The following primary outcomes are assessed:
Frequency by grade of solicited local reactogenicity adverse events (AEs) [Time Frame: Through 7 days post each vaccination]
Frequency by grade of solicited systemic reactogenicity adverse events (AEs) [Time Frame: Through 7 days post each vaccination]
Frequency by grade of unsolicited adverse events (AEs) [Time Frame: Through 28 days post each vaccination]
Frequency of Adverse Events of Special Interest (AESIs) [Time Frame: Day 1 through Day 478]. Including potentially immune-mediated medical conditions (PIMMCs), medically attended adverse events (MAAEs), and new onset chronic medical conditions (NOCMCs)
Frequency of clinical safety laboratory adverse events by severity grade [Time Frame: Through 7 days post each vaccination]. Parameters to be evaluated include: white blood cell count (WBC), hemoglobin (HgB), platelets (PLT), alanine aminotransferase (ALT), aspartate aminotransferase (AST), alkaline phosphatase (ALP), total bilirubin (T Bili), creatine kinase (CK), and creatinine (Cr)
Frequency of Serious Adverse Events (SAEs) [Time Frame: Day 1 through Day 478]

The following secondary outcomes are assessed:
Geometric mean fold rise from baseline in titer measured by a SARS-CoV-2 neutralization assay, for wild-type virus and emergent viral strains [Time Frame: Day 1 through Day 478]
Geometric mean fold rise from baseline in titer of receptor-binding domain (RBD) specific Immunoglobulin G (IgG) [Time Frame: Day 1 through Day 478]. Measured by an Enzyme-Linked Immunosorbent Assay (ELISA), for RBD from wild-type virus and emergent viral strains
Geometric mean fold rise from baseline in titer of Spike-specific Immunoglobulin G (IgG) [Time Frame: Day 1 through Day 478]. Measured by an Enzyme-Linked Immunosorbent Assay (ELISA), for spike protein from wild-type virus and emergent viral strains
Geometric mean titer measured by a SARS-CoV-2 neutralization assay, for wild-type virus and emergent viral strains [Time Frame: Day 1 through Day 478]
Geometric mean titer of receptor-binding domain (RBD) specific Immunoglobulin G (IgG) [Time Frame: Day 1 through Day 478]. Measured by an Enzyme-Linked Immunosorbent Assay (ELISA), for RBD from wild-type virus and emergent viral strains
Geometric mean titer of Spike-specific Immunoglobulin G (IgG) [Time Frame: Day 1 through Day 478]. Measured by an Enzyme-Linked Immunosorbent Assay (ELISA), for spike protein from wild-type virus and emergent viral strains
Percent of cells expressing a cytokine by cell type (CD4+ or CD8+), cytokine set (Th1 or Th2 cytokine for CD4+ and CD8+ cytokine for CD8+ or other combinations of interest) and peptide pool (covering spike and T cell epitope regions) [Time Frame: Day 1 through Day 478]. As determined by ICS
Percentage of subjects who seroconverted, for RBD from wild-type virus and emergent viral strains [Time Frame: Day 1 through Day 478]. Seroconversion defined as a 4-fold change in receptor-binding domain (RBD) specific IgG from baseline measured by ELISA. Including against emergent viral strains, e.g., B.1.1.7., as assessed by a range of assays measuring total Spike-specific Immunoglobulin G (IgG) (Enzyme-Linked Immunosorbent Assay (ELISA)-based) and function (neutralization, receptor-binding domain (RBD) binding, or similar) in serum
Percentage of subjects who seroconverted, for spike protein from wild-type virus and emergent viral strains [Time Frame: Day 1 through Day 478]. Seroconversion defined as a 4-fold change in Spike-specific Immunoglobulin G (IgG) from baseline measured by an Enzyme-Linked Immunosorbent Assay (ELISA). Including against emergent viral strains, e.g., B.1.1.7., as assessed by a range of assays measuring total Spike-specific Immunoglobulin G (IgG) (Enzyme-Linked Immunosorbent Assay (ELISA)-based) and function (neutralization, receptor-binding domain (RBD) binding, or similar) in serum
Percentage of subjects who seroconverted, for wild-type virus and emergent viral strains [Time Frame: Day 1 through Day 478]. Seroconversion defined as a 4-fold change in titer from baseline measured by a SARS-CoV-2 neutralization assay. Including against emergent viral strains, e.g., B.1.1.7., as assessed by a range of assays measuring total Spike-specific Immunoglobulin G (IgG) (Enzyme-Linked Immunosorbent Assay (ELISA)-based) and function (neutralization, receptor-binding domain (RBD) binding, or similar) in serum
Rate of spot-forming cell per million cells by peptide pool (covering spike and T cell epitope regions) [Time Frame: Day 1 through Day 478]. As determined by interferon (IFN) gamma Enzyme Linked Immunospot Assay (ELISpot)
Responder status, derived from the intracellular cytokine staining (ICS) cell counts for each set of applicable cytokines and each peptide pool [Time Frame: Day 1 through Day 478]. Covering spike and T cell epitope regions
Responder status, determined by interferon (IFN) gamma Enzyme Linked Immunospot Assay (ELISpot) for each peptide pool [Time Frame: Day 1 through Day 478]. Covering spike and T cell epitope regions
Th1/Th2 cytokine balance of T cell response [Time Frame: Through 28 days post boost vaccination]. By measuring interleukin (IL) 2, tumor necrosis factor (TNF) alpha, IL-4, IL-10, and IL-13 using a multiplexed cytokine assay with Enzyme Linked Immunospot Assay (ELISpot) supernatants in a subset of subjects

### XIV.K. SARS-CoV-2 Vaccine Clinical Assessment in Rhesus Macaques

SARS-CoV-2 vaccines were assessed in Rhesus Macaques non-human primates (NHP).

### Methods

Vector Construction: The ChAd68 nucleotide sequence was based on the wild-type sequence obtained by MiSeq (Ilumina sequencing) of virus obtained from the ATCC (VR-594). The sequence of a E1 (578-3404 bp)/E3 deleted virus (2,125-31,825 bp) was assembled into pUC19 from VR-594-derived and synthetic (SGI-DNA) fragments. An E4 deletion between E4ORF2-4 was introduced by PCR. A CMV promoter/enhancer with an SV40 polyA was introduced into the E1 region and the spike gBlock sequences introduced by Gibson assembly (Codexis) and transformed into Stb14 (Thermo Fisher) cells. Error free clones were selected by PCR and sequencing and plasmid DNA prepared at the Maxi-prep scale (Machery-Nagel). Furin and proline spike mutations, 2P or 6P, were introduced into the spike protein by overlapping PCR extension using primers to introduce the specific mutations. The pA68-E4d-Spike plasmids were linearized, purified using a Nucleospin kit (Machery-Nagel) and transfected into 2 mL of 293F cells (0.5 mL/mL) using TransIT-Lenti (Mirus bio). The virus was amplified, harvested and re-infected into 30 mL of 293F cells for 48-72 h. Cells and media were harvested and used to infect 400 mL of 293F cells. Cells were harvested after 48 hours and lysed by a freeze/thaw step (−80° C./37° C.) in 10 mM Tris pH 8.0/0.1% Triton-X100, and then purified by two successive rounds of CsCl gradient centrifugation. Virus bands were purified and dialyzed 3× into 1×ARM buffer (10 mM Tris pH 8.0, 25 mM NaCl, 2.5% glycerol). Viral particle concentration was determined by the Absorbance 260 nm method post lysis in 0.1% SDS and the infectious unit (IU) titer was determined by immunostaining. Spike sequences were PCR amplified and cloned into PacI/BstBI sites of a pUC02-VEE vector. Capped SAM was synthesized in vitro using HiScribe T7 Quick High Yield RNA Synthesis Kit (New England Biolabs) and purified using a RNeasy Maxi Kit (Qiagen) according to the manufacturer's protocol. SAM was subsequently encapsulated in a lipid nanoparticle (LNP) using a self-assembly process in which an aqueous solution of SAM is rapidly mixed with a lipid mixture in ethanol. RNA encapsulation efficiency was measured using Ribogreen RNA quantitation reagent (Thermo Fisher) and confirmed to be >95% in all batches analyzed. SAMLNP was formulated into a buffer containing 5 mM Tris (pH 8.0), 10% sucrose, 10% maltose.

Intracellular Cytokine Staining: Freshly isolated splenocytes were resuspended at a density of 5×10⁶ cells/mL in complete RPMI and following an overnight rest at 4° C., 1×10⁶ cells per well were distributed into v-bottom 96-well plates. Cells were pelleted and resuspended in 100 μL of complete RPMI containing an overlapping peptide pool containing 316 peptides (each 15 amino acids in length, 11 amino acid overlap) spanning the SARS-CoV-2 spike antigen, at a final concentration of 0.5 μg/mL per peptide (Genscript). A second well with DMSO only was used as a negative control for each sample. After 1 hour of incubation at 37° C., Brefeldin A (Biolegend) was added to a final concentration of 5 μg/mL and cells were incubated for an additional 4 hours. Following stimulation, cells were washed with PBS and stained with fixable viability dye (eBioscience). Extracellular staining was performed in FACS buffer (PBS+2% FBS+2 mM EDTA) with the following antibodies: CD4 (GK1.5, Biolegend), CD8 (53-6.7, BD). Cells were then washed, fixed and permeabilized with the eBiosciences Fixation/Permeabilization Solution Kit. Intracellular staining was then performed in permeabilization buffer with the following antibodies: IFNγ (XMG21.2, Invitrogen), TNFα (MP6-XT22, eBiosciences), IL2 (JES6-5H4, eBiosciences), IL4 (11B11, Biolegend), IL10 (JES5-16E3, Biolegend). Samples were collected on a Cytoflex LX (Beckman Coulter). Analysis of flow cytometry data was performed using FlowJo software.

NHP Studies: Study was conducted in compliance with all relevant local, state and federal regulations and were approved by the Battelle Institutional Animal Care and Use Committee (IACUC). 30 Chinese-origin male and female rhesus macaques (M. mulatta) >2.5 years old were housed at Battelle (Columbus, Ohio). NHP were vaccinated with either ChAd-Spike(V2)-F2P (Group 1-5×10¹¹ VP, study day 0; Group 2-5×10¹¹ VP, study day 28), SAM-Spike(V2)-F2P (Group 1-30 μg, study day 42; Group 3-30 μg, study days 14 and 42; Group 4-10 μg, study days 14 and 42; Group 5-3 μg, study days 14 and 42), or PBS (Group 6, study days 0 and 42). All injections were bilateral intramuscular, 0.5 mL per leg (1 mL total) to the thigh. Note that immunizations were staggered to enable challenge immunization on study day 70 for all groups. Each group was divided into two sub-groups and study initiated one week apart, to enable two staggered challenge groups. Animals were challenged with SARS-CoV-2 (strain USA-WA1/2020) via the intratracheal (0.5 mL) and intranasal (0.25 mL per nostril) routes with a target dose of ˜1.6×10⁶ PFU (undiluted). Serum was collected using serum separator tubes and whole blood collected in cell preparation tubes and peripheral blood mononuclear cells isolated and cryopreserved in CryoStorCS solution (Sigma Aldrich).

ELISpot assays: IFNγ ELISpot assays were performed using pre-coated 96-well plates (Mabtech, Monkey IFNγ ELISPOT PLUS, ALP or Mouse IFNγ ELISPOT PLUS, ALP) following manufacturer's protocol. For NHP, frozen PBMCs were thawed at 37° C. and then rested overnight in RPMI+10% FBS. 1×10⁵ PBMCs were plated per well in triplicate with a single overlapping peptide pool spanning spike from the N to C terminus (GenScript, 15 amino acid length, 11 amino acid overlap, 314 peptides total) at final concentration of 1 ug/mL per peptide and incubated overnight at 37° C. in RPMI+10% FBS. For mouse studies, freshly isolated splenocytes were stimulated overnight with either two (˜120 peptides/each) or eight different overlapping peptide pools (36-40 peptides each) spanning the SARS-CoV-2 spike antigen, at a final concentration of 1 μg/mL per peptide (GenScript). Splenocytes were plated in duplicate at 1×10⁵ cells per well and 2.5×10⁴ cells per well (mixed with 7.5×10⁴ naïve cells) for each stimulus. DMSO only was used as a negative control for each sample. Plates were washed with PBS and then incubated with anti-monkey or anti-mouse IFNγ mAb biotin (Mabtech) for two hours, followed by an additional wash and incubation with Streptavidin-ALP (Mabtech) for one hour. After final wash, plates were incubated for ten minutes with BCIP/NBT (Mabtech) to develop the immunospots. Wells were imaged and spots enumerated using AID reader (Autoimmun Diagnostika). Samples with replicate well variability (Variability=Variance/(median+1) greater than 10 and median greater than 10 were excluded. Spot values were adjusted based on the well saturation according to the formula: AdjustedSpots=RawSpots+2*(RawSpots*Saturation/(100−Saturation). Each sample was background corrected by subtracting the average value of the negative control wells. Data was normalized to spot forming units (SFU) per 1×10⁶ cells by multiplying the corrected spot number by 1×10⁶/cell number plated. Data processing was performed using the R programming language and graphed using GraphPad Prism. Statistical analysis was performed in GraphPad Prism.

Pseudovirus Neutralization Assay: Mouse and human convalescent serum samples (courtesy of Helen Chu, University of Washington) were assessed by Nexelis (Laval, Quebec). NHP serum samples were assessed by Gritstone bio (Emeryville, CA) using the same pseudovirus, controls, reagents and protocol. Pseudotyped virus particles were made using a genetically modified Vesicular Stomatitis Virus from which the glycoprotein G was removed (VSVΔG). The VSVΔG virus was transduced in HEK293T cells previously transfected with the spike glycoprotein of the SARS-CoV-2 coronavirus (Wuhan strain) for which the last 19 amino acids of the cytoplasmic tail were removed (ΔCT). The generated pseudovirus particles (VSVΔG-Spike ΔCT) contain a luciferase reporter which can be quantified in relative luminescence units (RLU). Heatinactivated serum samples were serially diluted (7-serial 2-fold dilution) in a 96-well plate and a pre-determined amount of pseudotyped virus (corresponding to between approximately 75,000 and 300,000 RLU/well) was applied to the plate and incubated with serum/plasma to allow binding of the neutralization antibodies to the pseudotyped virus. After the incubation of the serum/plasma-pseudotyped virus complex, the serum/plasmapseudotyped virus complex was transferred to the plate containing Vero E6 cells (ATCC). Test plates were incubated at 37° C. with 5% C02 overnight. Luciferase substrate was added to the plates which were then read using a plate reader detecting luminescence. The intensity of the light being emitted is inversely proportional to the amount of anti-SARS-CoV-2 neutralizing Spike antibodies bound to the VSVΔG-Spike ΔCT particles. Each microplate was read using a luminescence microplate reader (SpectraMax). The dilution of serum required to achieve 50% neutralization (NT50) when compared to a non-neutralized pseudoparticle control was calculated for each sample dilution and the NT50 is interpolated from a linear regression using the two dilutions flanking the 50% neutralization.

Microneutralization Assay: The microneutralization assay was performed at Battelle Memorial Institute (Columbus, Ohio) to assess the neutralizing antibody titer in serum samples collected from NHPs following vaccination and post-challenge. The virus neutralization titer is expressed as the reciprocal value of the highest dilution of the serum which still inhibited virus replication. All serum samples were analyzed in duplicate. Briefly, 2-fold serial dilutions of heat-inactivated serum sample were pre-incubated with the virus for 60 minutes at 37° C. The virus/serum mixture was then added to a 90 to 100% confluent monolayer of Vero E6 cells (BEI, Cat. No. NR-596) in 96-well plates and incubated for two days at 37° C. with 5% C02. Following incubation, the inoculum was removed, and monolayers were incubated for 30 minutes in 80% cold acetone to allow cell fixation. Plates were incubated with anti-nucleocapsid protein primary antibody cocktail (clones HM1056 and HM1057) (EastCoast Bio, North Berwick, ME) for 60 minutes at 37° C. (Battelle Memorial Institute, Patent No. 63/041,551 Pending, 2020). The plates were washed and the secondary antibody (goat antimouse IgG Horse Radish Peroxidase (HRP) conjugate; Fitzgerald, North Acton, MA) was added to the wells, and the plates were incubated for 60 minutes at 37° C. After the plates were washed, the substrate was added, and the plates were incubated at 37° C. Stop solution was added, and the plates were read for optical density at 405 nm wavelength. Neutralizing activity is defined as at least 50% reduction in signal from the virus only (VC) wells relative to cells control (CC) wells following the formula [(average VC−average CC)/2]+average CC. The median neutralizing titer (MN50) was calculated using Spearman-Kärber analysis method.

S1 IgG ELISA: 96-well QuickPlex plates (Meso Scale Discovery, Rockville, MD) were coated with 50 μL of 1 μg/mL SARS-CoV-2 S1 (ACROBiosystems, Newark, DE), diluted in DPBS (Corning, Corning, NY), and incubated at 4° C. overnight. Wells were washed three times with agitation using 250 μL of PBS+0.05% Tween-20 (Teknova, Hollister, CA) and plates blocked with 150 μL Superblock PBS (Thermo Fisher Scientific, Waltham, MA) for 1 hour at room temperature on an orbital shaker. Test sera was diluted at appropriate series in 10% species-matched serum (Innovative Research, Novi, MI) and tested in single wells on each plate. Wells were washed and 50 μL of the diluted samples were added to wells and incubated for 1 hour at room temperature on an orbital shaker. Wells were washed and incubated with 25 μL of 1 μg/mL SULFO-TAG labeled anti-species antibody (MSD), diluted in DPBS+1% BSA (Sigma-Aldrich, St. Louis, MO), for 1 hour at room temperature on an orbital shaker. Wells were washed and 150 μL Read Buffer T (MSD) added. Plates were run immediately using the QPlex SQ 120 (MSD) ECL plate reader. For each sample, the endpoint titer was calculated as the reciprocal serum dilution at 2-fold the average background value for the plate, interpolated by a linear regression using the two highest dilutions that gave values greater than 2-fold the average background.

IFNα2α ELISA: NHP serum samples were analyzed for levels of IFNα2α using a MSD U-PLEX Biomarker assay (catalog number K15068L-2), according to the manufacturer's instructions. Analyte concentration (pg/mL) was calculated using serial dilutions of known standards. Each animal and timepoint was run in technical duplicates.

Viral RNA RT-qPCR (Nucleocapsid Protein (N1) Genomic Analysis): Briefly, RNA was isolated using the Indispin QIAcube HT Pathogen Kit (Indical Bioscience, Germany) on the QIAcube HT instrument (Qiagen, Germany). The isolated RNA was then evaluated in RT-qPCR using the TaqMan Fast Virus 1-step Master Mix (Thermo Fisher Scientific) on a QuantStudio Flex 6 Real-Time PCR System (Applied Biosystems; Foster City, CA). The primers and probe were specific to the SARS-CoV-2 nucleocapsid gene, corresponding to the N1 sequences from the Centers for Disease Control and Prevention (CDC) 2019-Novel Coronavirus (2019-nCoV) Real-Time RT-PCR Diagnostic Panel (www.cdc.gov/coronavirus/2019-ncov/lab/rt-pcr-panel-primer-probes.html) except that the probe quencher was modified to Non-Fluorescent Quencher-Minor Groove Binder (NFQMGB) (Thermo Fisher Scientific). A standard curve comprised of synthetic RNA containing the target sequence from SARS-CoV-2 isolate WA1 sequence (GenBank Accession Number MN985325.1) (Bio-Synthesis, Inc.; Lewisville, TX) was included on each PCR plate for absolute quantitation of SARS-CoV-2 RNA copies in each sample. Thermocycling conditions were as follows: Stage 1—50° C. for 5 min for one cycle; Stage 2—95° C. for 20 sec for one cycle; Stage 3—95° C. for 3 sec and 60° C. for 30 sec for 40 cycles. Data analysis was performed using the QuantStudio 6 software-generated values (total copies per well of each sample) and additional calculations to determine SARS-CoV-2 N1 copies per mL of fluid.

Viral RNA RT-qPCR (Envelope Protein (E) Subgenomic Analysis): Following isolation and evaluation using the N1 genomic assay, the isolated RNA was then evaluated as described above using primers and probes specific to the SARS-CoV-2 E gene based on previously described sequences, and the reverse primer and probe sequences previously described (Integrated DNA Technologies, Iowa). A standard curve comprised of synthetic RNA containing the target sequence from SARS-CoV-2 isolate WA1 sequence (GenBank Accession Number MN985325.1) (Bio-Synthesis, Inc.; Lewisville, TX) was included on each PCR plate for absolute quantitation of SARS-CoV-2 copies in each sample. Thermocycling conditions were as follows: Stage 1—50° C. for 5 min for one cycle; Stage 2—95° C. for 20 sec for one cycle; Stage 3—95° C. for 3 sec and 60° C. for 30 sec for 40 cycles. Data analysis was performed using the QuantStudio 6 software-generated values (total copies per well of each sample) and additional calculations to determine SARS-CoV-2 E gene subgenomic (Esg) RNA copies per mL of fluid.

### Results

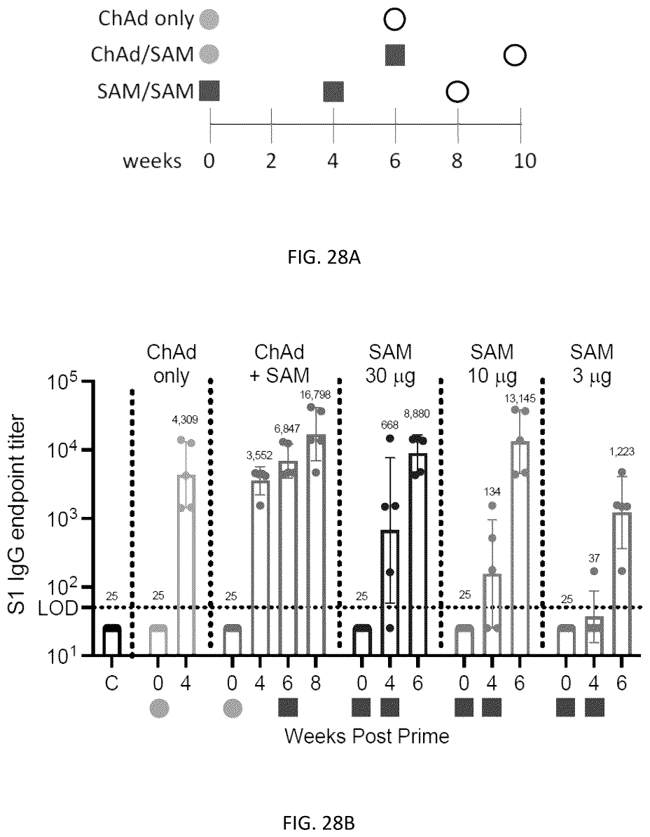

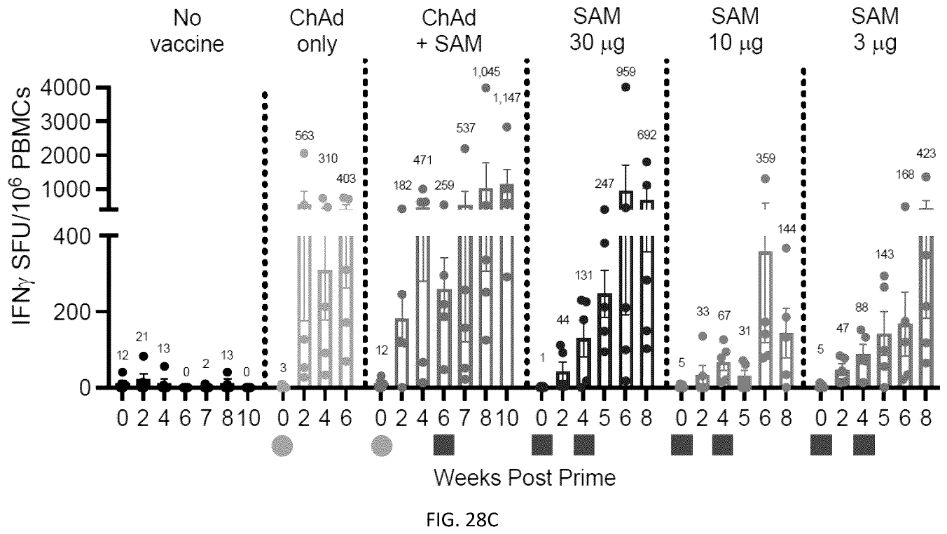

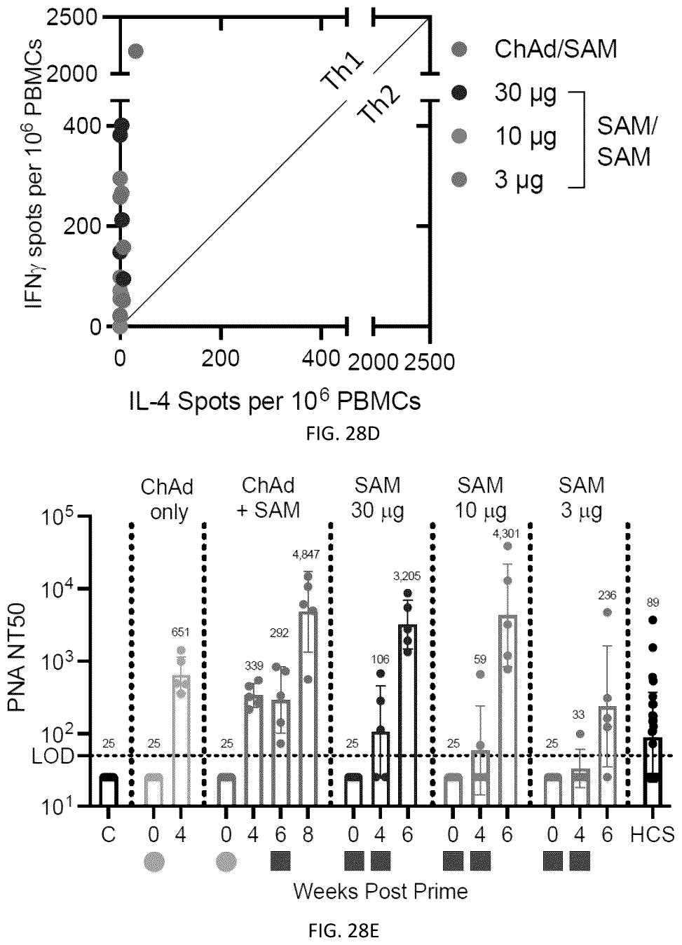

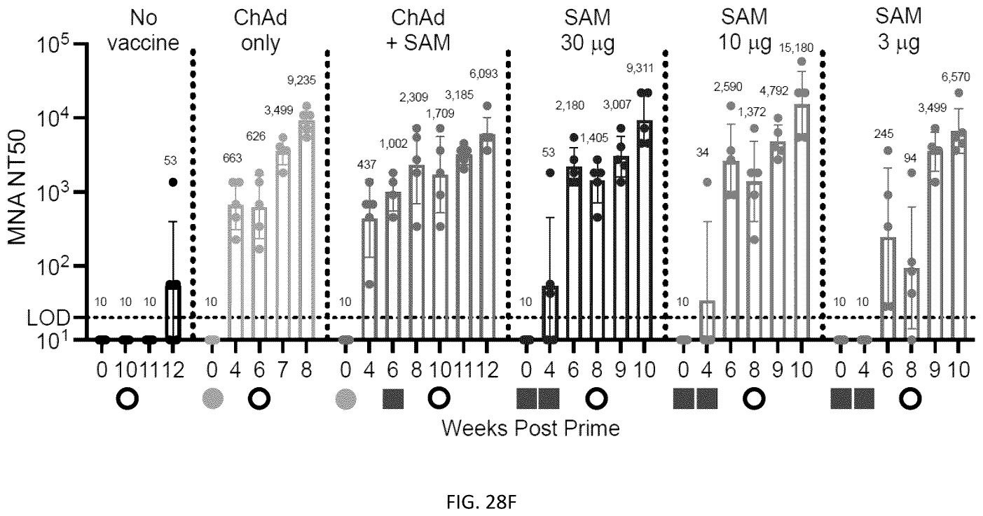

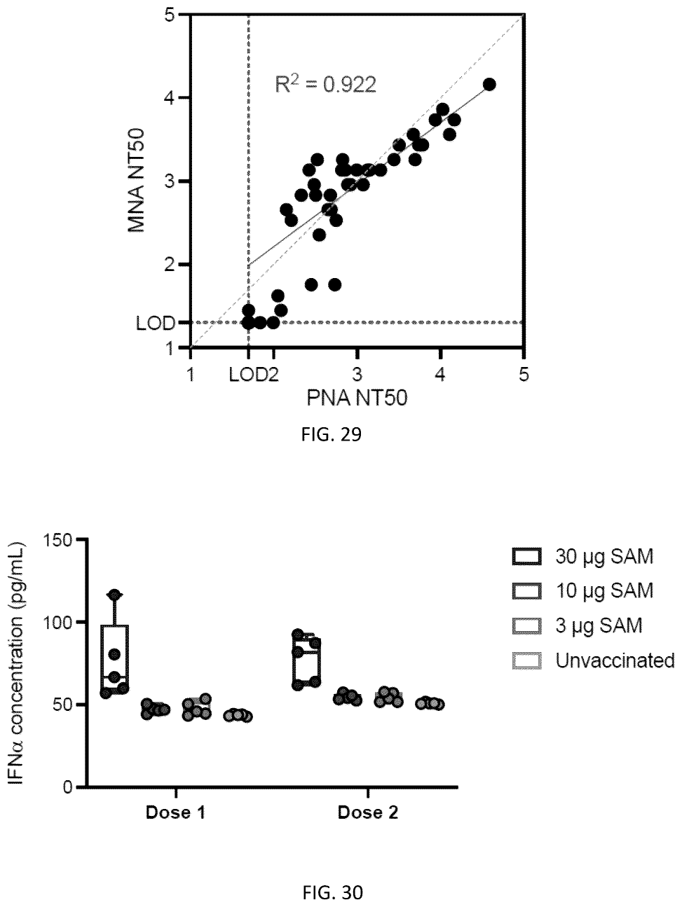

Groups of 5 rhesus macaques were immunized intramuscularly twice with SAM-Spike(V2)-F2P (4-week interval) at either 3, 10 or 30 μg (FIG. 28A). Spike S1—specific IgG antibody titers were observed in some NHP following a single SAM immunization in a dose dependent manner and were increased to high levels in all NHP following a 2nd SAM immunization at all doses tested (GMT 8,880, 13,145 and 1,223 at 30, 10 and 3 μg, respectively, FIG. 28B). Spike-specific T cell responses, assessed by overnight IFNγ ELISpot using a single spike peptide pool, were detected at all dose levels following two immunizations (FIG. 28C). T cell response was significantly increased compared to unvaccinated control animals for all 3 dose groups (p<0.05 for all 3 dose groups by Mann-Whitney comparison of peak T cell values) and were not significantly different between the dose groups. ELISpot analysis demonstrated strong IFNγ, but minimal IL-4, responses one-week following the boost immunization in all vaccinated animals, indicating a TH1-biased response (FIG. 28D). Strong neutralizing antibody titers were detected following two immunizations of SAM by both a pseudovirus neutralization assay (PNA) and live virus microneutralization assay (MNA), which strictly correlated (FIG. 28E; FIG. 28F; FIG. 29). High and similar neutralizing antibody titers were detected in all NHP (10/10) at both the 30 and 10 μg dose level (NT50 GMT 2,180 and 2,590 2-weeks post boost by MNA), demonstrating comparable immune response with lower doses of SAM. Lowering the dose of SAM further to 3 μg resulted in overall lower, but clearly detectable neutralizing antibody titers in 5/5 NHP (by MNA) compared with the 10 and 30 μg dose (GMT 245 2-weeks post boost) (FIG. 28F). In addition, while potent spike-specific immune responses were observed at all dose levels, there was no increase in serum IFNα levels at 8 hours following each SAM immunization at doses ≤10 μg, suggesting that innate immune pathways are not activated at lower doses of SAM (FIG. 30).

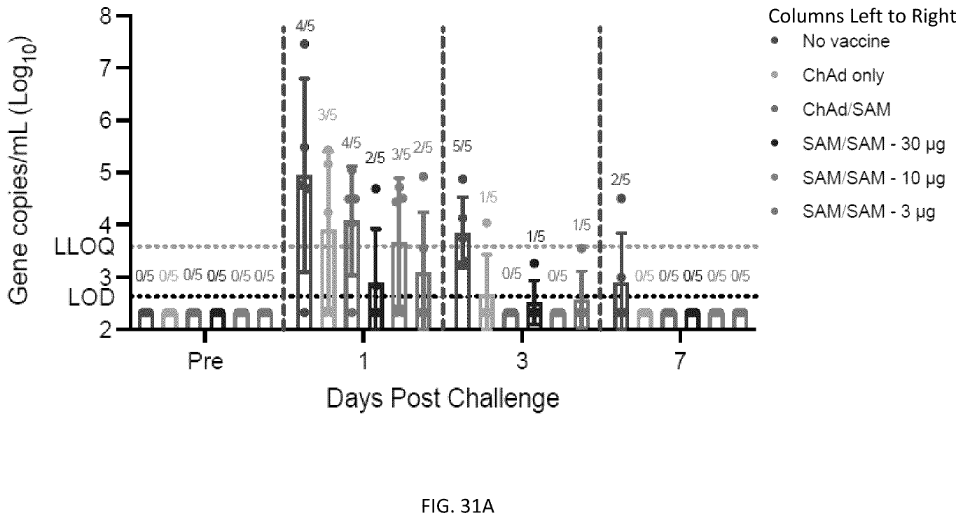

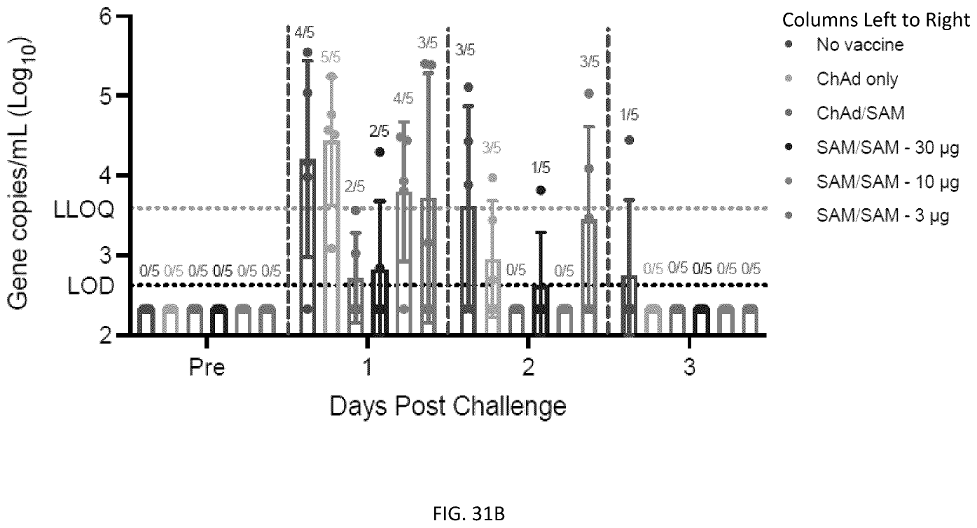

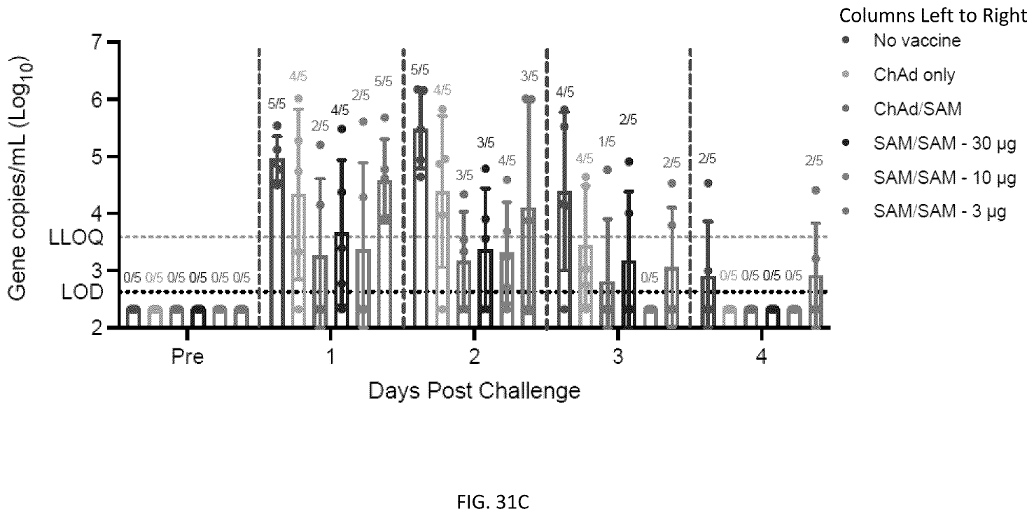

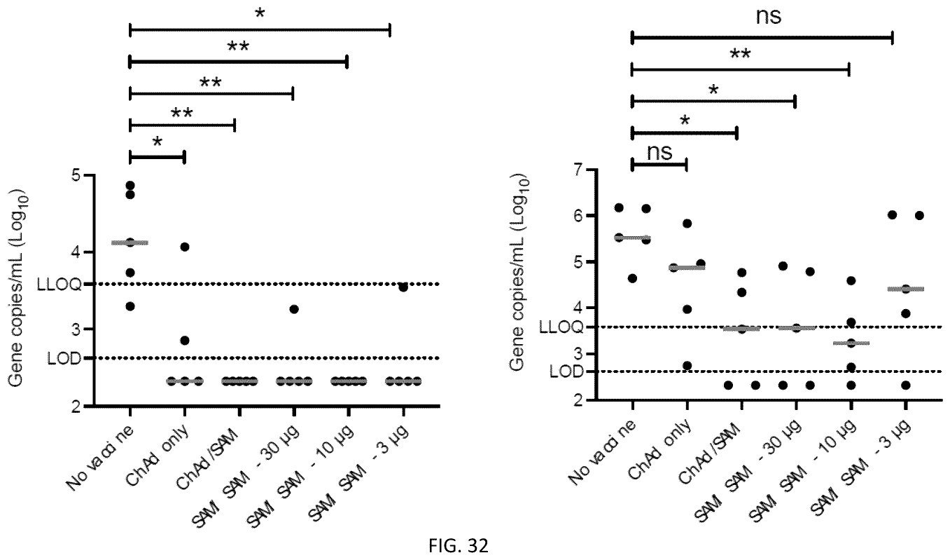

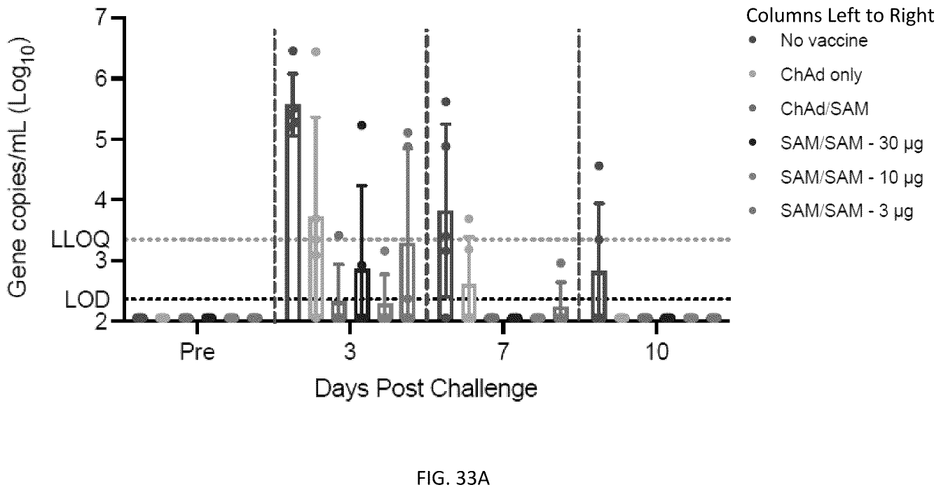

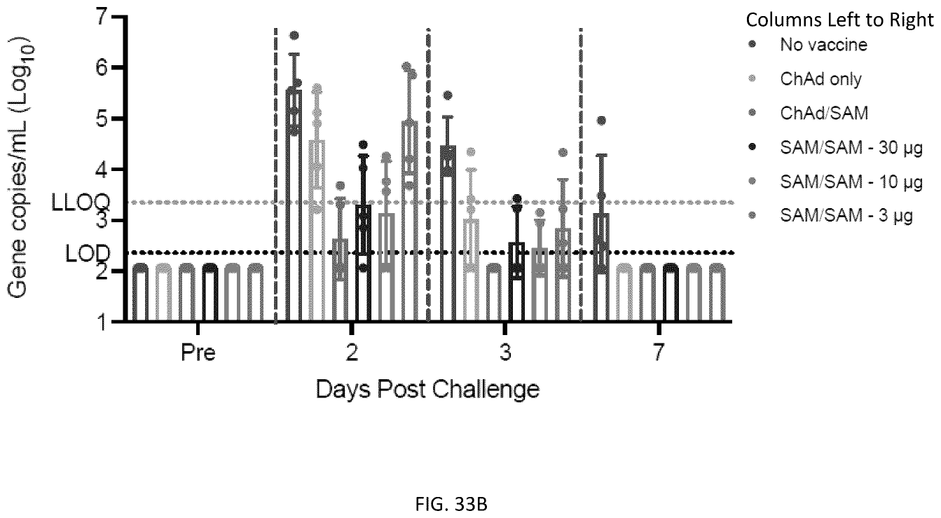

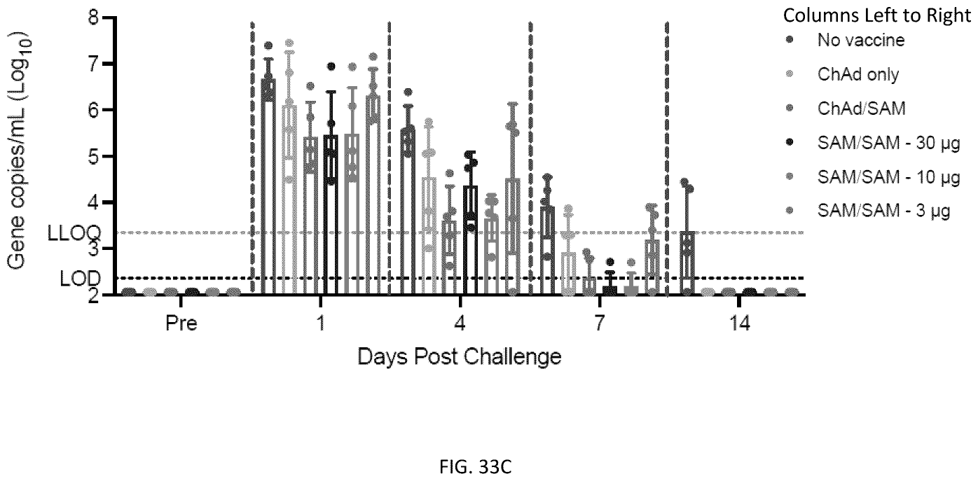

At 4-weeks following the 2nd SAM immunization (week 8) (FIG. 28A), all rhesus macaques were challenged with ˜1.6×10⁶ plaque-forming units of SARS-CoV-2 (strain USA-WA1/2020), split equally between the intratracheal and intranasal routes. Consistent with previous studies of SARS-CoV-2 challenge in rhesus macaques, none of the vaccinated or control animals showed clinical signs of illness. Neutralizing antibody titers were detected in 3/5 phosphate buffered saline (PBS) injected (control) animals 2-weeks post challenge (NT50 GMT 53). An increase in titers was observed 1- or 2-weeks post challenge in all SAM vaccinated animals, to much higher levels than those observed in control animals (NT50 GMT 9,311, 15,180 and 6,570 for SAM at 30, 10, and 3 μg, respectively, at 2-weeks post challenge), indicating an anamnestic response (FIG. 28F). To assess protective efficacy provided by the vaccine, viral load was assessed by PCR in bronchial alveolar lavage (BAL) fluid, oropharyngeal swabs, and nasal swabs at various times post challenge. Both total genomic RNA and subgenomic RNA (sgRNA) were assessed to quantify input virus and replicating virus, respectively. Three days after challenge, only one of five animals at the 30 μg and 3 μg dose levels and zero of five animals in the 10 μg group had detectable subgenomic RNA in BAL fluid, as compared to five of five animals in the control group. By day 7, none of the animals in any vaccine treated group had detectable sgRNA in the BAL, as compared to 2/5 in the control group, demonstrating protection from viral replication at all dose levels (FIG. 31A). Comparison of peak viral titer for each animal between day 3 and day 10 post challenge by Mann-Whitney analysis demonstrated significant protection from viral replication at all dose levels compared to control animals (p=0.008(**), 0.008(**) and 0.016(*) at 30, 10 and 3 μg, respectively, FIG. 32). In the oropharyngeal swab at day 2, none of the animals in the 10 μg dose group had detectable sgRNA, compared to one of five in the 30 μg SAM group, three of five in the control group and 3 μg SAM group (FIG. 31B). On day 3, none of the animals in the SAM 10 μg dose group and two out of five in the SAM 30 and 3 μg groups had detectable sgRNA in nasal swabs, compared to four out of five in the control group (FIG. 31C), demonstrating significantly decreased viral replication in nasal swabs at the 30 and 10 μg dose levels (p=0.032(*), 0.008(**) and 0.222(ns), peak viral load for each animal between day 2 and day 10 post challenge compared to control by Mann-Whitney analysis, at 30, 10 and 3 μg, respectively, FIG. 32). Furthermore, total RNA levels were lower in BAL, oropharyngeal and nasal swabs in all SAM vaccinated animals compared to control animals, with improved viral clearance observed with the 30 and 10 μg of SAM compared with 3 μg SAM (FIG. 33A-C). Collectively, this data demonstrate protective immunity and efficacy in all SAM vaccinated animals, even at the lowest dose of 3 μg, with the 10 μg and 30 μg dose levels providing comparable and more effective protection, similar to what was previously described with mRNA vaccines at a dose of 100 μg.

Since many individuals have already been vaccinated with a SARS-CoV-2 specific adenovirus vaccine, the potency of the SAM vaccine was further explored in a ChAd/SAM heterologous prime/boost vaccine regimen (5×10¹¹ VP ChAd prime with 30 μg SAM boost, 6-week interval, FIG. 28A), which have been shown to generate potent and durable antigen-specific CD8+ T cell responses in ongoing oncology clinical trials (data not shown). Strong spike S1 IgG antibody titers were observed following a single ChAd immunization and were increased 2.5-fold following SAM boost immunization (GMT 6,847 at 6-weeks post prime and 16,798 2-weeks post boost) to similar titers as those observed following two SAM immunizations at the 30 and 10 μg dose levels (FIG. 28B). Strong spike-specific IFNγ, TH1 biased, T cell responses were detected 2-weeks after a single immunization of ChAd that were slightly higher than those observed after a single SAM immunization (at any dose), demonstrating the potency of the ChAd vaccine to rapidly drive high titer T cell responses (FIG. 28C, D). In addition, T cell responses were increased in 5/5 NHP following the SAM boost (30 μg) immunization to similar levels as those observed following two SAM immunizations (10 or 30 μg), demonstrating the potency of the SAM vaccine as either a boost vector post ChAd prime or as a homologous prime/boost vaccine regimen. Potent neutralizing antibody titers were detected in 10/10 NHP by both PNA and MNA assays following a single ChAd immunization (live virus MNA NT50 GMT 663 4-weeks post prime), again demonstrating the potency of the ChAd vaccine vector. Following SAM (30 μg) boost immunization, titers were increased 2.3-fold (NT50 GMT 2,309 2-weeks post boost), to similar levels as those observed following two doses of 30 or 10 μg SAM (FIG. 28E, F), demonstrating comparable potency of the heterologous and homologous prime/boost regimens after two vaccinations even at low doses of the SAM homologous prime/boost vaccination regimen. Rhesus macaques were challenged with SARS-CoV-2 at 4-weeks following the SAM boost immunization (week 10) and 6-weeks following ChAd prime for the ChAd single immunization group (FIG. 28A). A similar increase in neutralizing antibody titers was observed in both groups (FIG. 28F). Three days after the challenge, only one of five animals in the ChAd only and zero of five animals in the ChAd/SAM heterologous group had detectable subgenomic RNA in BAL fluid, as compared to five of five animals in the control group. By day 7, none of the animals in any vaccine treated group had detectable sgRNA in the BAL, as compared to 2/5 in the control group (FIG. 31A), demonstrating significantly decreased viral replication in the BAL for both a single ChAd immunization and heterologous prime/boost (p=0.032(*) and p=0.008(**), respectively, peak viral load for each animal between day 3 and day 10 post challenge compared to control by Mann-Whitney analysis, FIG. 32). On day 3, one out of five NHP in the heterologous prime/boost group had detectable sgRNA in the nasal swab, compared to four out of five in the ChAd only and control groups (FIG. 31C), demonstrating increased protective efficacy with heterologous prime/boost compared to a single ChAd immunization (p=0.012(*) with ChAd/SAM and p=0.158(ns) with ChAd only, peak viral load for each animal between day 2 and day 10 post challenge compared to control by Mann-Whitney analysis, FIG. 32). Collectively, these data demonstrate decreased viral replication and therapeutic benefit provided by the vaccines in all animals, with the 30 μg and 10 μg of SAM homologous prime/boost and the heterologous regimen providing complete protection and the 3 μg SAM homologous prime/boost offering slightly decreased immune response but clearly providing protection to animals challenged with live virus.

### XIV.L. Further SARS-CoV-2 Vaccine Clinical Assessment in Human Subjects

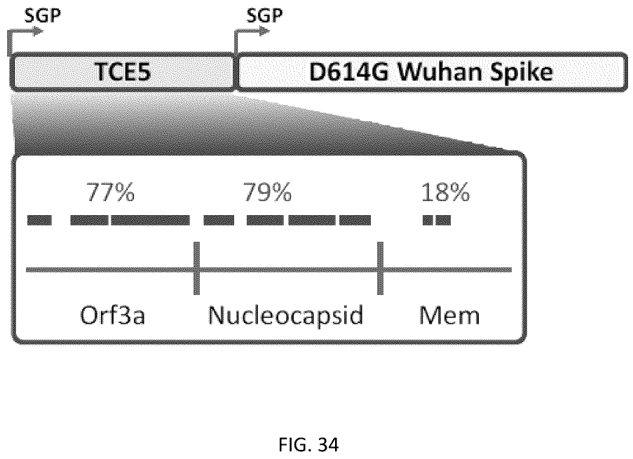

A clinical study was conducted in a Phase 1 trial to evaluate the safety, immunogenicity, and reactogenicity of a self-amplifying mRNA (samRNA) prophylactic vaccine boost against SARS-CoV-2 in previously vaccinated healthy adults 18 years and older. Specifically, vaccine candidate GRT-R910 (SAM-SGP1-TCE5-SGP2-CTSpike_{G}F2P (SEQ ID NO:27983)) was evaluated for improving and broadening the cell-mediated immune response (which includes genes outside of spike) and inducing immunity against more conserved genes and may be key to long-term, durable protection. A schematic of the vaccine construct and its coverage is shown in FIG. 34.

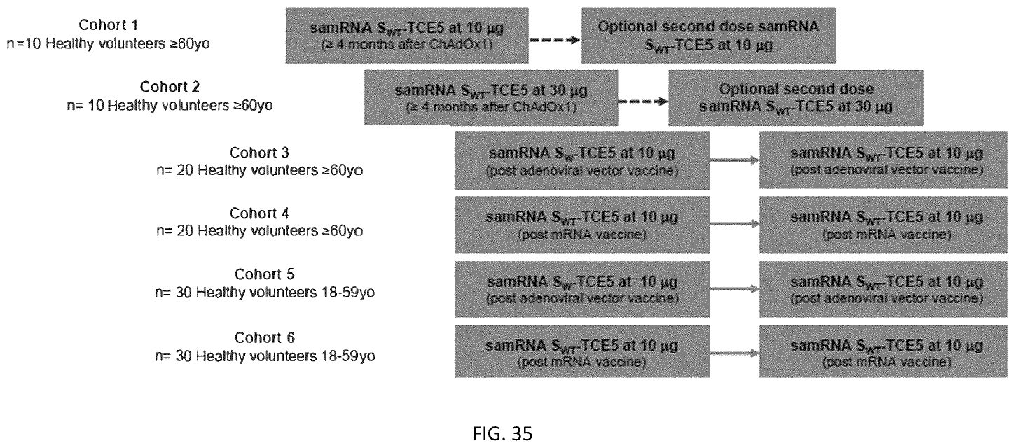

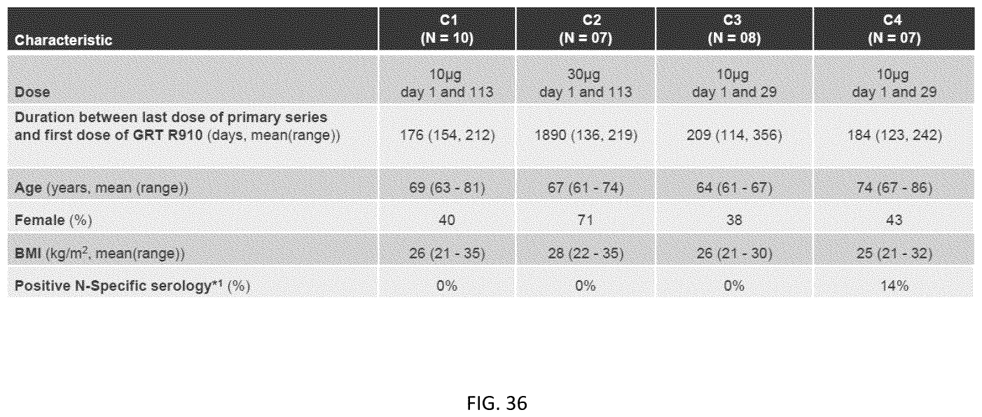

An open-label study (GO-009), conducted in the UK, of a self-amplifying mRNA (samRNA) vaccine encoding for wild type (WT) Spike (S) and highly conserved non-S CD8+ T cell epitopes (GRT-R910). A schematic of the cohorts for the clinical trial is shown in FIG. 35. A summary of the participant demographics in shown in FIG. 36.

GRT-R910 was administered as 1 or 2 boost vaccinations after prior vaccination with an authorized adenovirus or mRNA SARS-CoV-2 vaccine. The first two cohorts assessed 10 μg and 30 μg doses of GRT-R910 in older (≥60 years of age) adults who had received the primary ChAdOx1 series (AstraZeneca). Subsequent cohorts assessed two boost doses of 10 μg in older and younger adults who have received an adenovirus or mRNA vaccine. PBMC samples were collected for immunological assessment at different visits from day 1 up to day 365. The primary objectives were safety and reactogenicity and secondary objectives include cellular and humoral immunogenicity post-GRT-R910 vaccination(s).

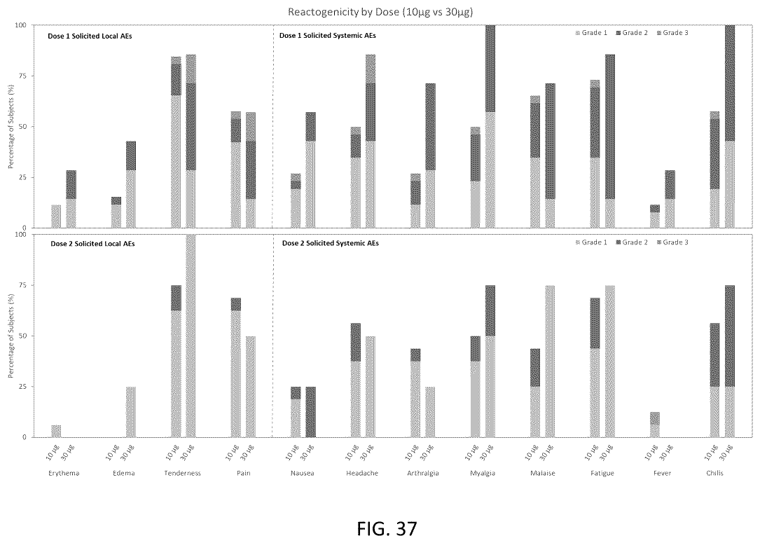

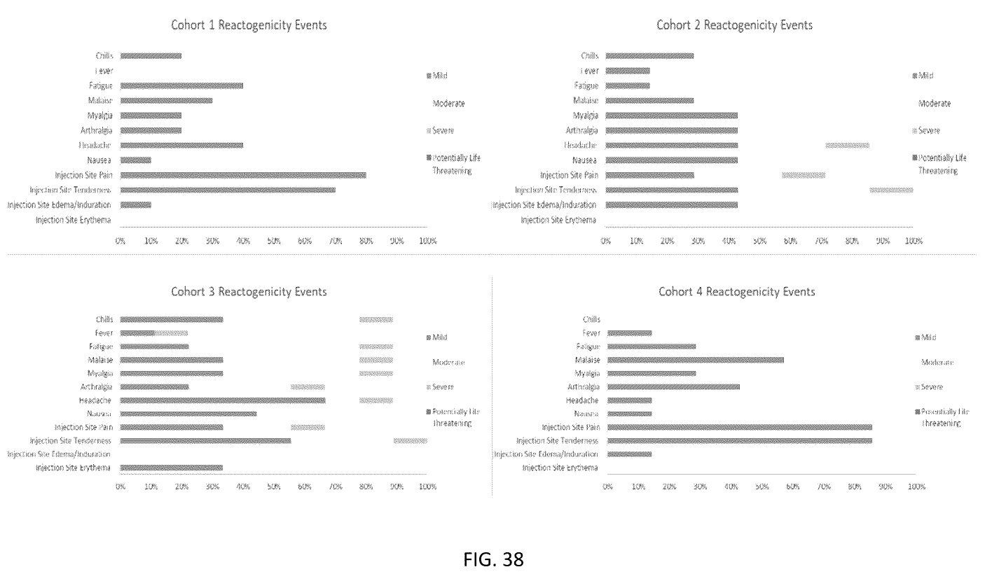

Preliminary safety and reactogenicity from cohorts 1-4 (older adults) was assessed, with summaries shown as a comparison between 10 μg and 30 μg doses following dose 1 (FIG. 37 top panel) and dose 2 (FIG. 37 bottom panel), and broken down across cohorts (FIG. 38). For local reactogenicity events, events were transient in nature where the most common events were grade 1 or 2 injection site pain and tenderness with, with one subject in the 30 μg dose cohort having grade 3 events of injection site pain, tenderness, and local induration. For systemic reactogenicity events, events were transient in nature where where (1) in the 10 μg cohort, the most common events were grade 1 or 2 pyrexia, fatigue, and headache; (2) in the 30 μg cohort, the most common events were grade 1 or 2 pyrexia fatigue, headache, malaise, and nausea; and (3) one subject in the 30 μg cohort had events of grade 3 fatigue, malaise, and headache while an additional subject had an event of grade 3 fatigue. Overall, the results demonstrate the 10 μg and 30 μg doses were well tolerated in healthy adults (≥60 years old).

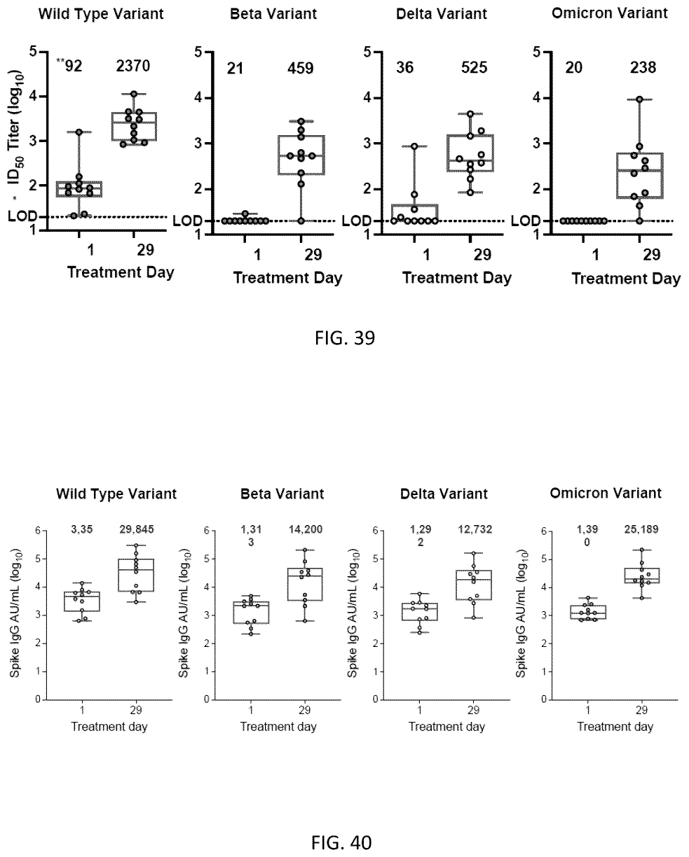

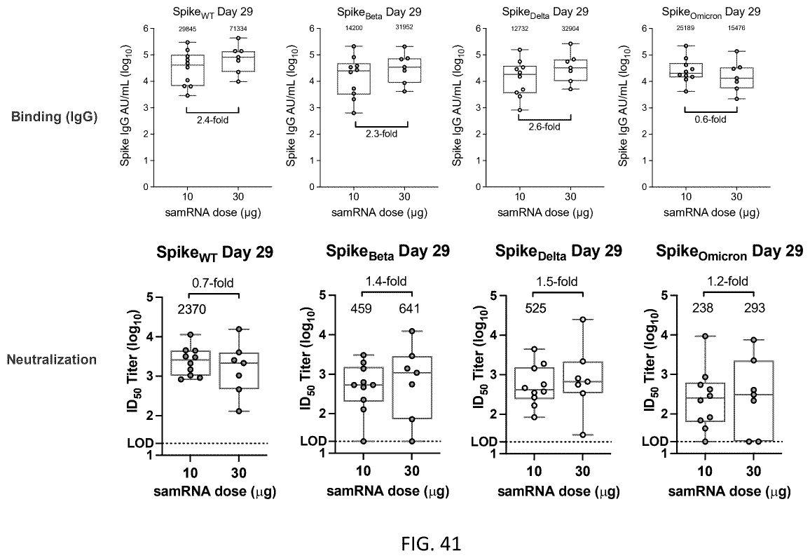

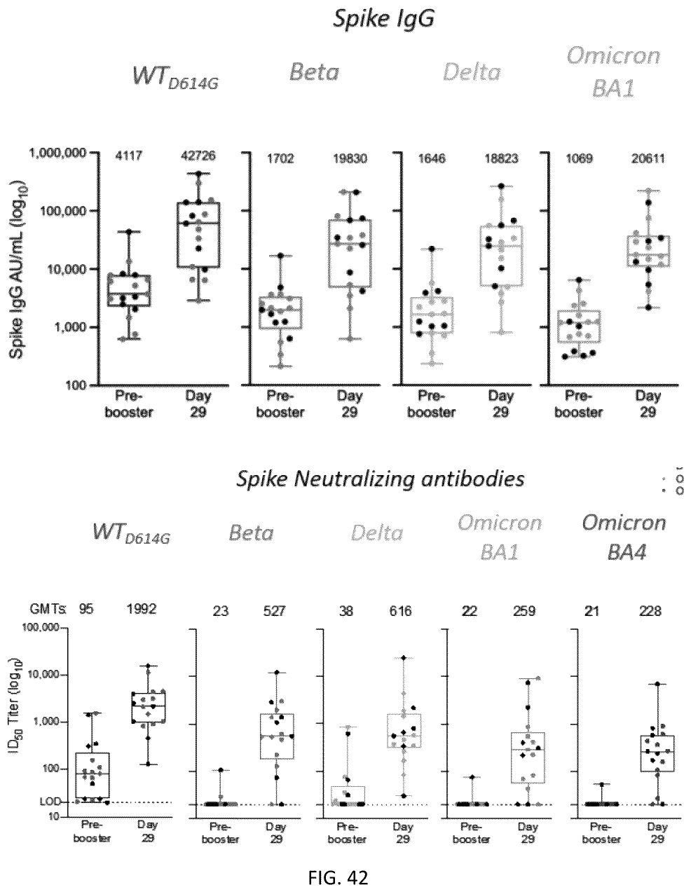

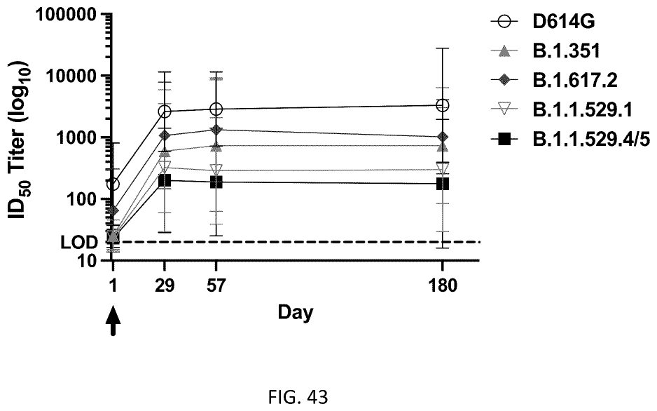

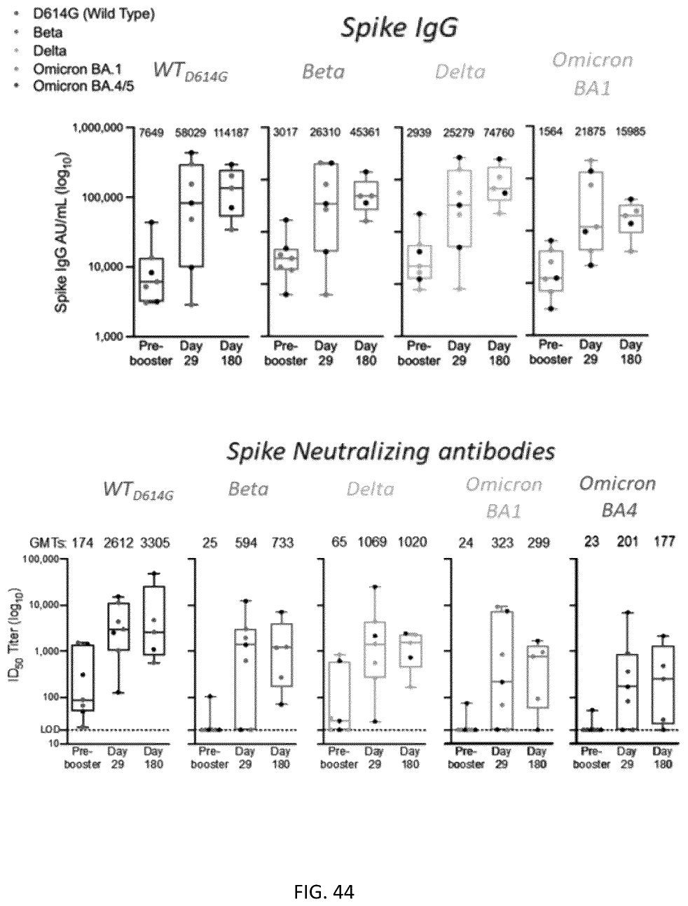

Initial immunogenicity data post the first boost data from cohorts 1-2 were assessed. As shown in FIG. 39, a single 10 μg samRNA boost stimulated a broad and potent nAb response against Wild Type, Beta, Delta, and Omicron variants of SARS-CoV-2 in healthy adults (≥60 years old). The nAb response against wild type was similar to previously reported values for a 100 μg of mRNA-1273 (Moderna) after a ChAdOx1 (AstraZeneca) primary series. As shown in FIG. 40, a single 10 μg samRNA boost also stimulated a broad anti-Spike IgG response against Wild Type, Beta, Delta, and Omicron variants of SARS-CoV-2 in healthy adults (≥60 years old). As shown in FIG. 41, comparing single doses of 10 μg or 30 μg samRNA resulted in similar titer levels for Spike IgG (top panel) and nAb (bottom panel). As shown in FIG. 42, comparing pre and post boost titer levels for single doses of 10 μg or 30 μg samRNA resulted in increased binding and neutralizing antibodies against WTD614G Spike and variants of concern Beta, Delta, and Omicron at 4 weeks for Spike IgG (top panel) and nAb (bottom panel). As shown in FIG. 43 and FIG. 44, neutralizing antibodies against multiple SARS-CoV-2 variants of interest persisted for at least 6 months following a single boost of 10 μg or 30 μg in healthy adults (≥60 years old) demonstrating a durable neutralizing antibody response.

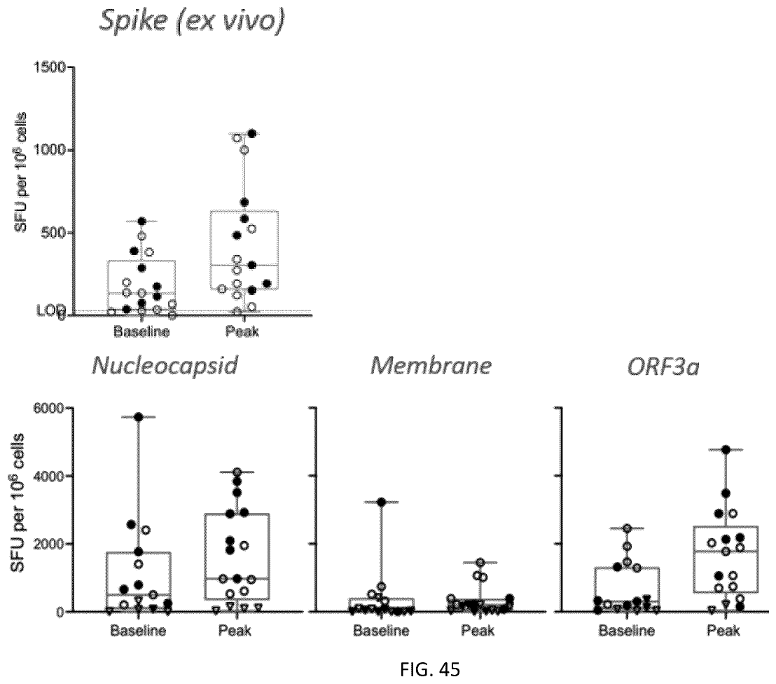

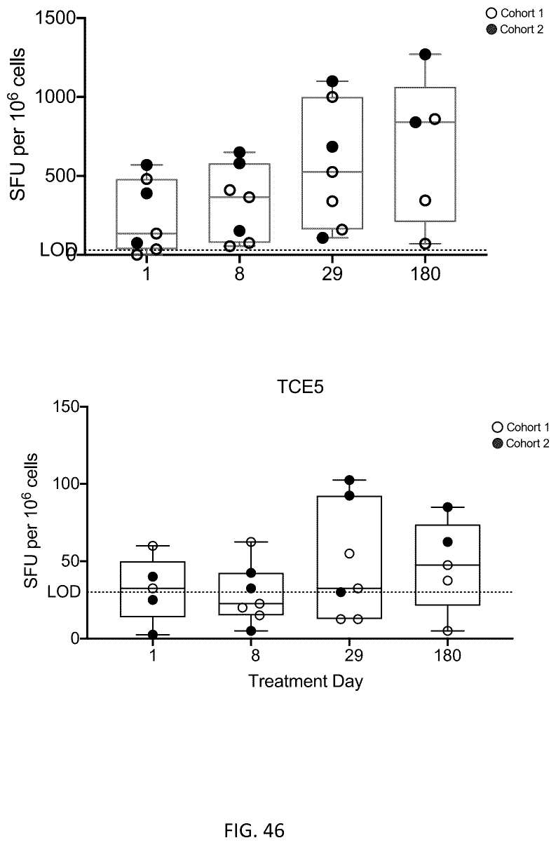

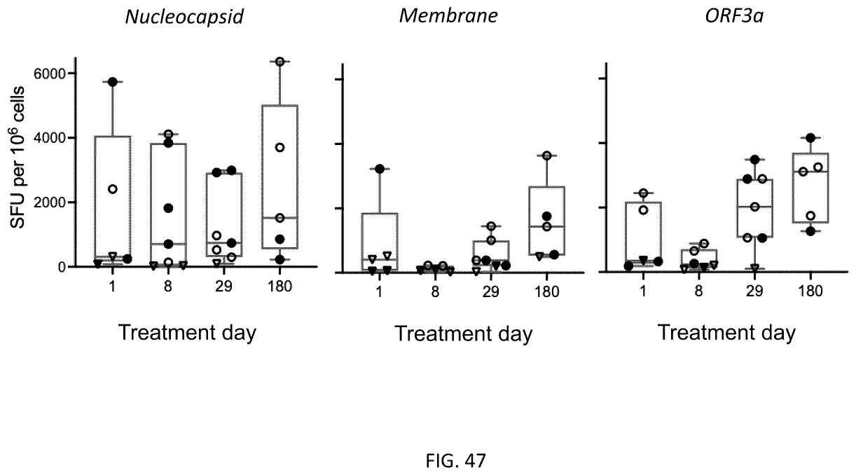

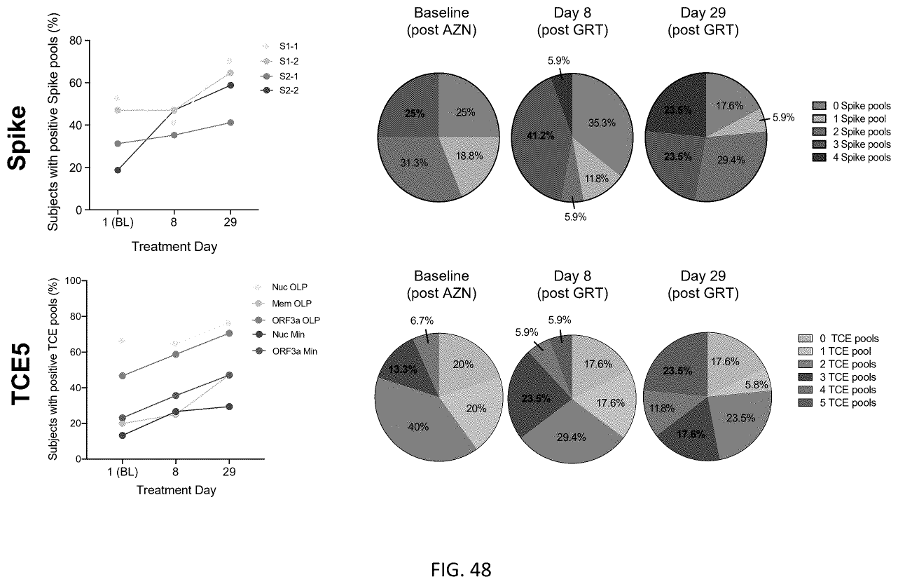

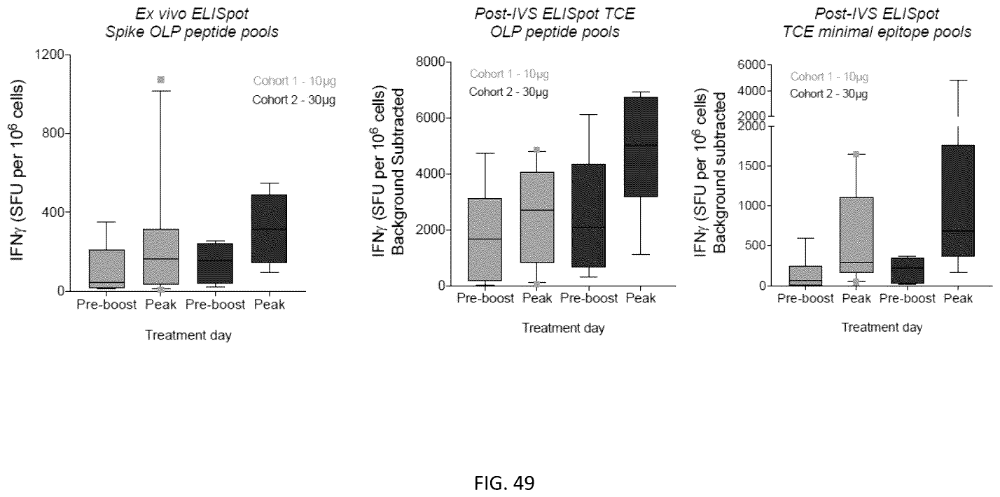

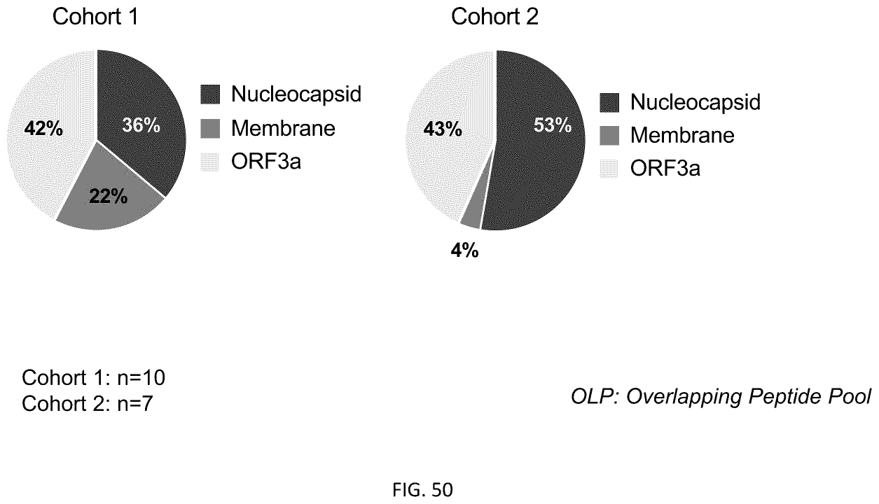

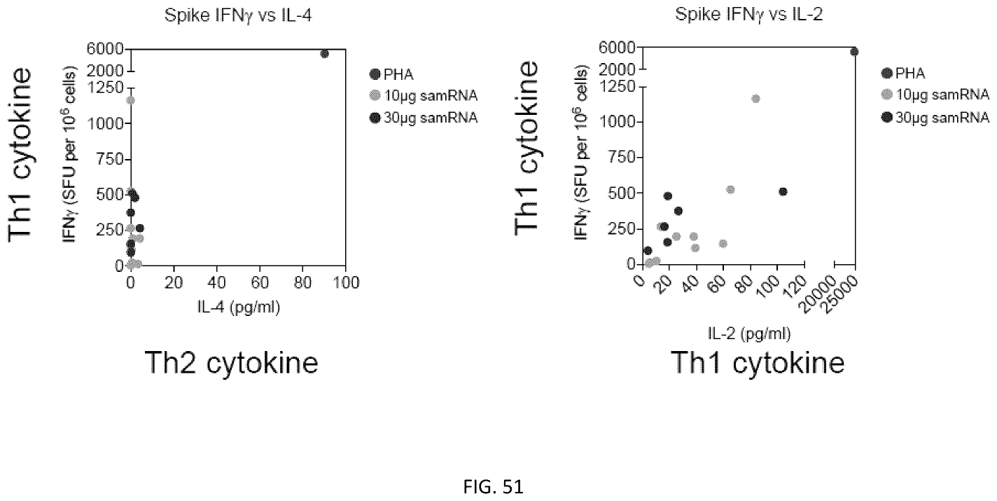

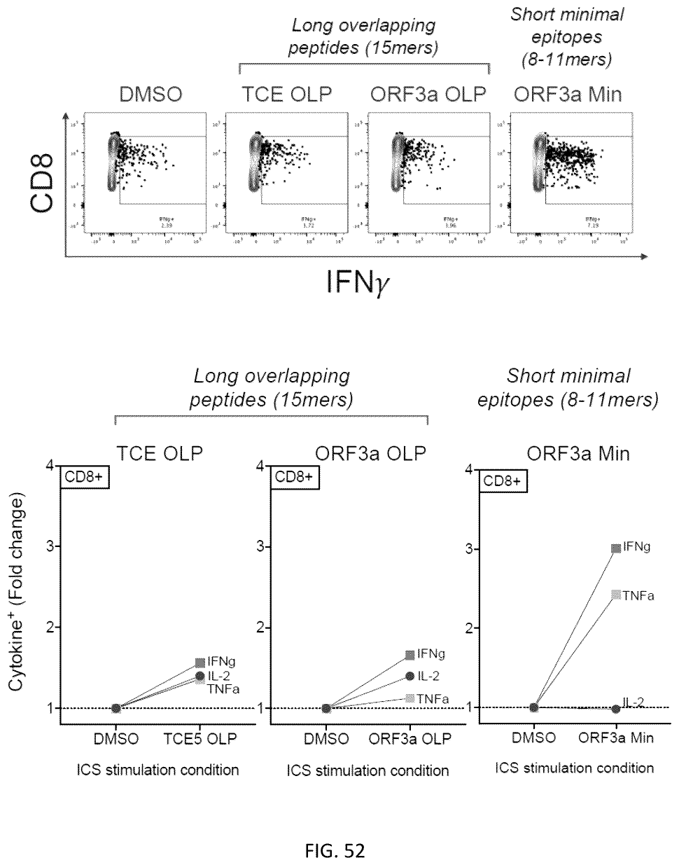

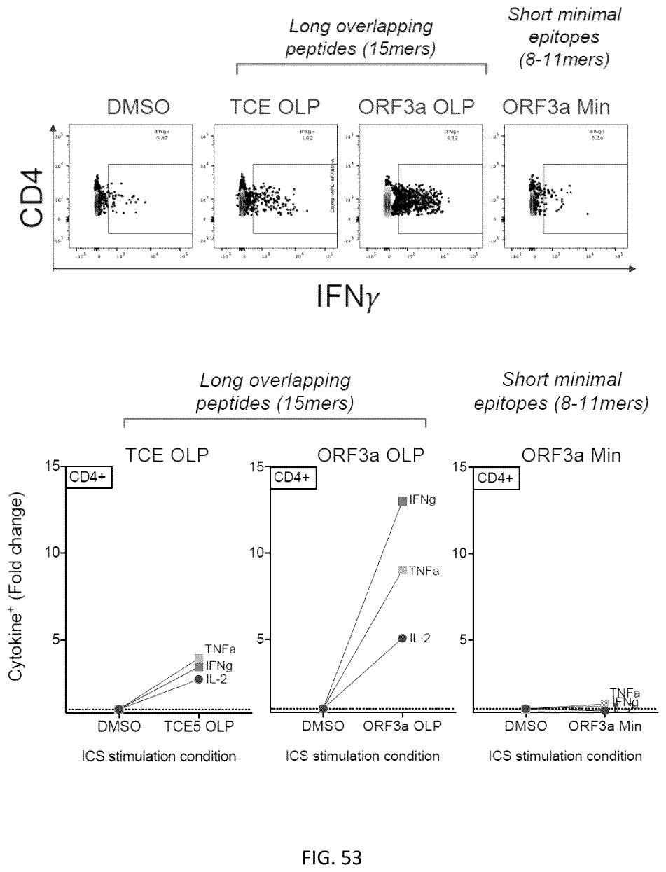

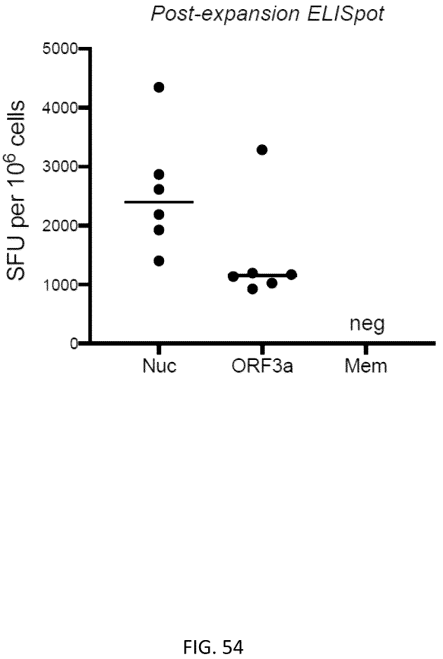

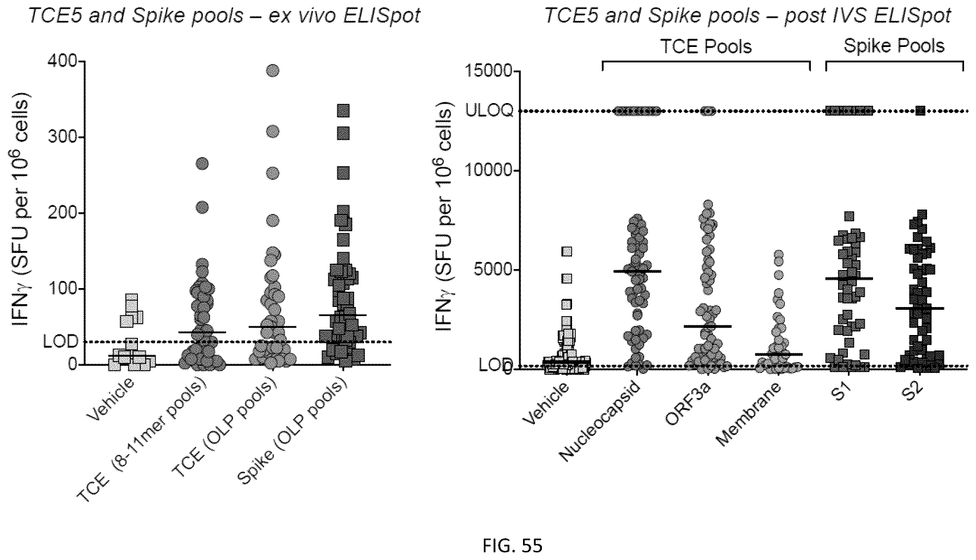

T cell responses were also evaluated. As shown in FIG. 45, a single boost with the samRNA increased T cell responses against Spike and non-Spike T cell epitope (TCE) regions included in the vaccine 1-4 weeks after a single 10 μg or 30 μg dose. As shown in FIG. 46, samRNA boost demonstrated an increased and durable ex vivo Spike and TCE5 response both 4 weeks and 6 months post samRNA boost, including samRNA boosting Spike primed T-cells and priming TCE-specific T-cells. As shown in FIG. 47, T cell responses against Nucleocapsid, Membrane, and ORF3a were maintained for at least 6 months following a single dose of samRNA, both 10 μg and 30 μg doses. As shown in FIG. 48, a single boost of samRNA increased breadth of both Spike- and TCE-specific T cell responses relative to the baseline responses in previously vaccinated adults (≥60 years of age), as shown by an increased breadth of response to different T cell epitope pools. As shown in FIG. 49, there was no clear dose response for ex vivo Spike T cell responses between the 10 μg and 30 μg doses, while there were slightly higher post-IVS TCE5 T cell responses with the 30 μg samRNA dose. As shown in FIG. 50, the 10 μg and 30 μg doses demonstrated similar breadth in response against Nucleocapsid, Membrane, and ORF3a. As shown in FIG. 51, the Spike-specific T Cell responses following samRNA administration were Th1 biased, as demonstrated by driving IFNγ and IL-2, but not IL-4 responses. As shown in FIG. 52, a single 10 μg dose of samRNA stimulated CD8+ T cell responses to TCE and ORF3a OLP pools and a ORF3a minimal epitope pool, in particular that stimulation with a short (8-11 mer) ORF3a peptide pool stimulated IFNγ and TNFα responses in CD8+ T cells. As shown in FIG. 53, a single 10 μg dose of samRNA stimulated CD4+ T cell responses to TCE and ORF3a OLP peptide pools, in particular that stimulation with long (15mer) ORF3a peptide pool drove IFNg, TNFa, and IL-2 responses in CD4+ T cells. As shown in FIG. 54, T cell responses to Nucleocapsid and ORF3a components of TCE5 in pre-pandemic donors suggest pan-coronavirus elements of the vaccine, in particular ex vivo ELISpot had low or no response (not shown), short antigen-specific expansion revealed cross-reactive memory T cells to Nucleocapsid and ORF3a regions. As shown in FIG. 55, T cell responses to TCE5 components were detected both ex vivo (left panel) and post expansion (right panel) in convalescent donors, indicating the relevance of predicted T cell epitopes.

Overall, GRT-R910 (SAM-SGP1-TCE5-SGP2-CTSpike_{G}F2P) was well tolerated when administered after a primary series of adenovirus or mRNA SARS-CoV-2 vaccine in healthy older adults and GRT-R910 as a boost dose after a primary series of Vaxzevria stimulated durable, broadly cross-reactive anti-Spike antibody titers, as well as durable T cells targeting both Spike and non-Spike epitopes.

### XIV.M. Further SARS-CoV-2 Vaccine Clinical Assessment in Human Subjects

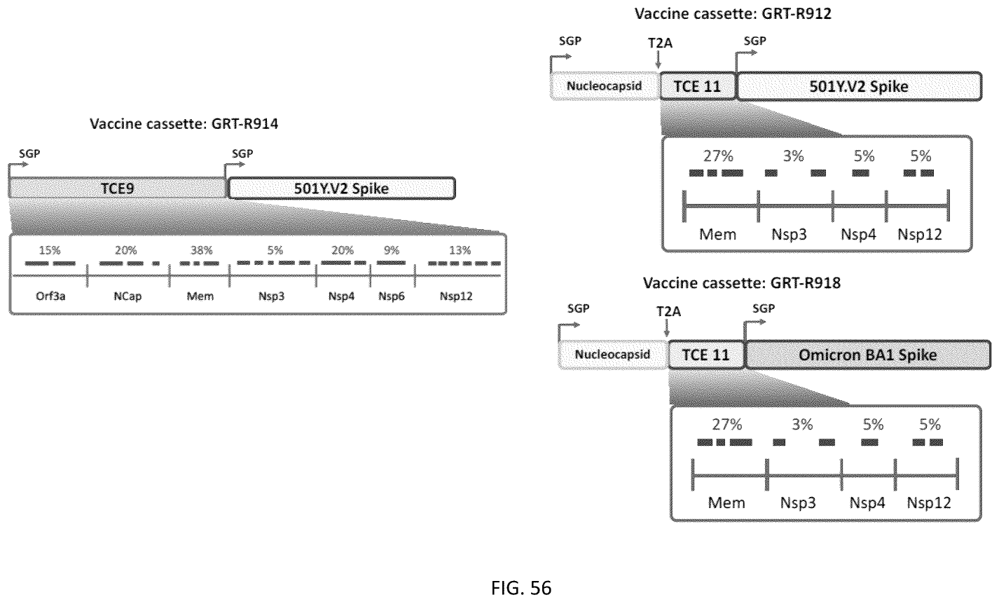

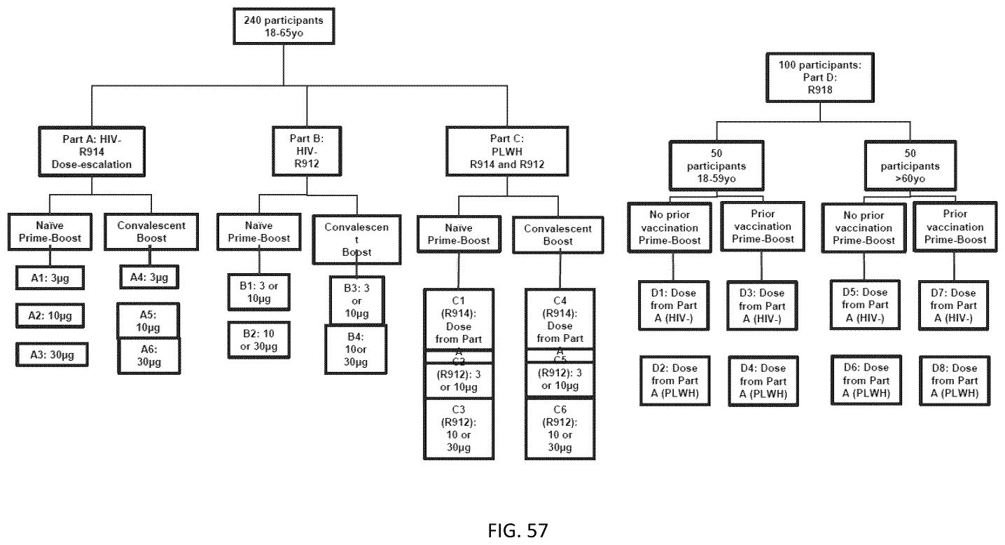

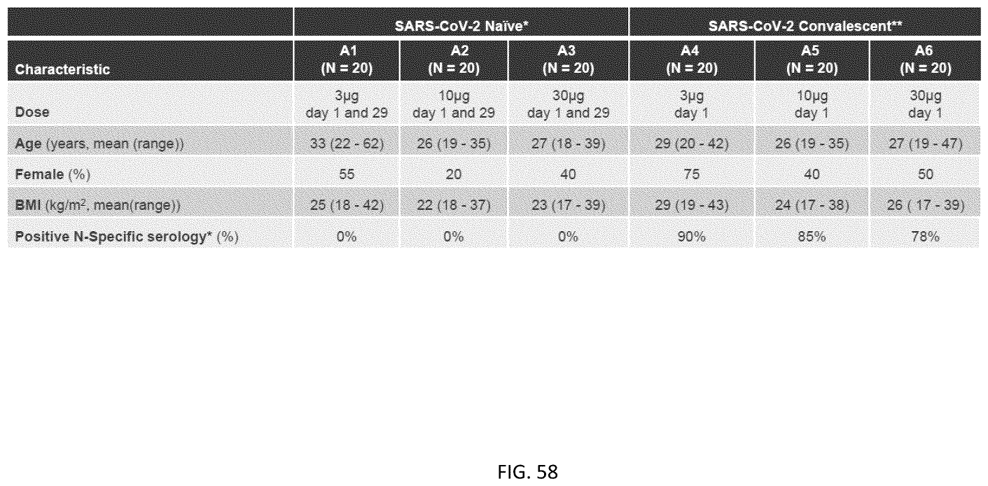

A Phase 1 SARS-CoV-2 vaccine study was designed to assess the safety and tolerability of GRT-R912 (N-TCE11-Spike-beta [SARS-CoV-2 “beta” is also referred to as 501Y.V2]; SEQ ID NO: 27981), GRT-R914 (TCE9-Spike-beta; SEQ ID NO: 27982), GRT-R918 (SAM-Nuc-TCE11-SpikeB.1.1.529 sequence; SEQ ID NO: 27976), and GRT-R920 (GRT-918 with Omicron Spike encoding sequences substituted with Omicron BA5 subisolate Spike encoding sequences) administered as prime and/or boost in healthy adult participants and people living with HIV. A schematic of the vaccine constructs and their coverage is shown in FIG. 56. A schematic of the cohorts for the clinical trial is shown in FIG. 57. A summary of the participant demographics in shown in FIG. 58. The study population skewed towards younger participants, with generally well-balanced covariates. Subjects with no history of SARS-CoV-2 infection or symptoms consistent with SARS-CoV-2 infection together AND a negative SARS-CoV-2 (N-specific) antibody test at pre-screening were considered SARS-CoV-2 naïve. Subjects with a history of confirmed SARS-CoV-2 infection OR a positive SARS-CoV-2 (N-specific) antibody test at pre-screening were considered SARS-CoV-2 convalescent.

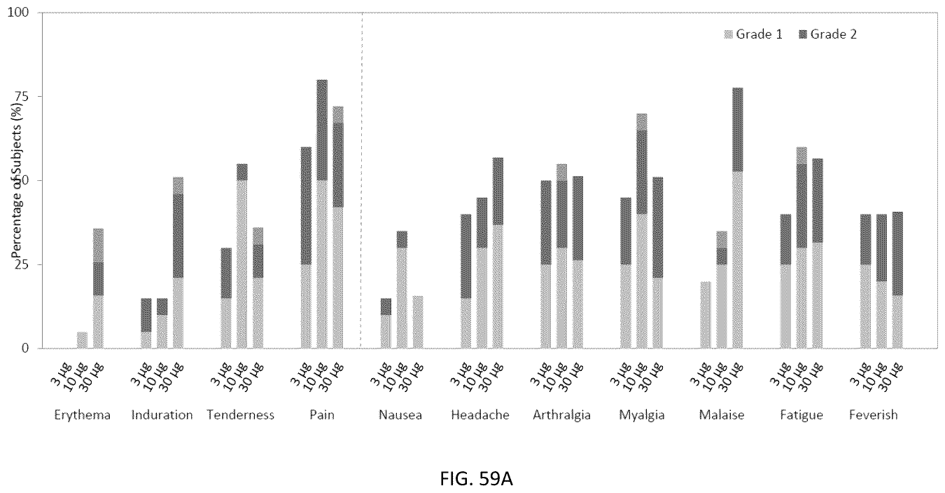

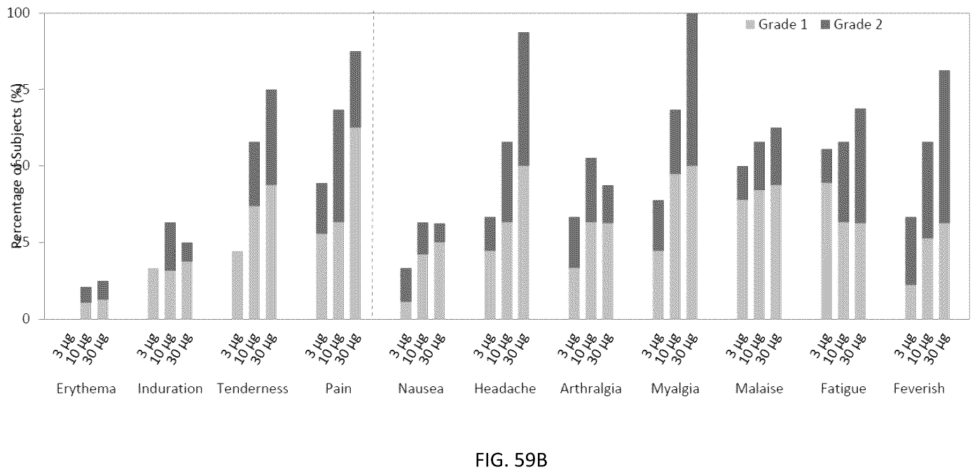

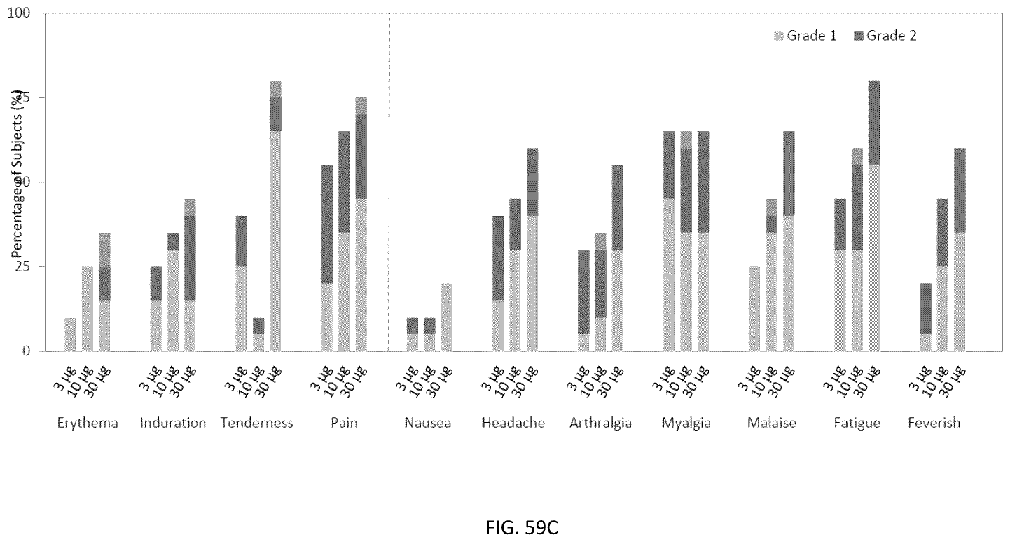

Preliminary safety and reactogenicity was assessed for GRT-R914 (TCE9-Spike-beta; SEQ ID NO: 27982), with summaries shown for “naïve” participants following dose 1 (FIG. 59A) and dose 2 (FIG. 59B), and in “convalescent” participants following a single dose (FIG. 59C). The results show 3 μg, 10 μg, and 30 μg doses of GRT-R914 were well tolerated, with reactogenicity slightly higher at 30 μg dose compared to lower doses. In SARS-CoV-2 convalescent subjects: (1) The majority of local and systemic solicited AEs were grade 1 or grade 2 and transient in nature at all three-dose levels and resolved within eight days or less; (2) Grade 3 events occurred in one subject at the 10 μg dose level and lasted for one day; and grade 3 events occurred in two subjects at the 30 μg dose level, lasting for four and two days respectively; (4) At the 30 μg dose level, two solicited reactogenicity events continued as grade 1 AEs after vaccination day eight and resolved by day ten. In SARS-CoV-2 naive (negative N-Specific serology) subjects: (1) The majority of local and systemic solicited AEs were grade 1 or grade 2 and transient in nature at all three-dose levels and resolved within eight days or less Grade 3 reactogenicity events occurred in a single subject at first dose and lasted one day at 3 μg dose; (2) Grade 3 reactogenicity events occurred in two subjects at 30 μg and lasted one day and two days respectively; (3) At 10 μg dose, solicited reactogenicity events continued in two subjects as AEs after eight days of 2nd vaccination dose and resolved on day ten and day nine respectively.

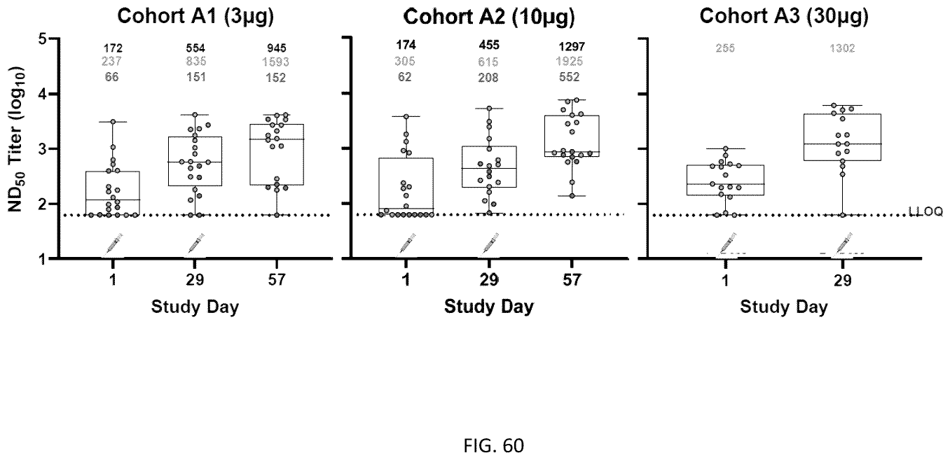

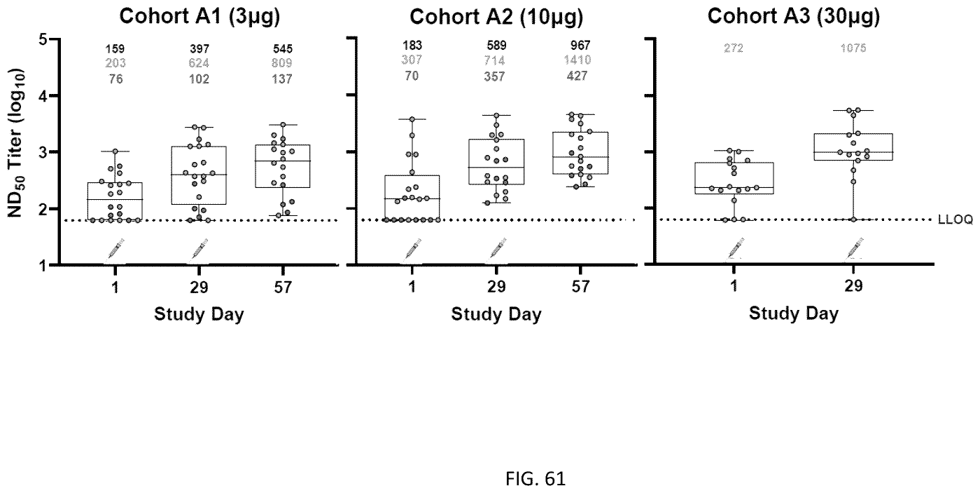

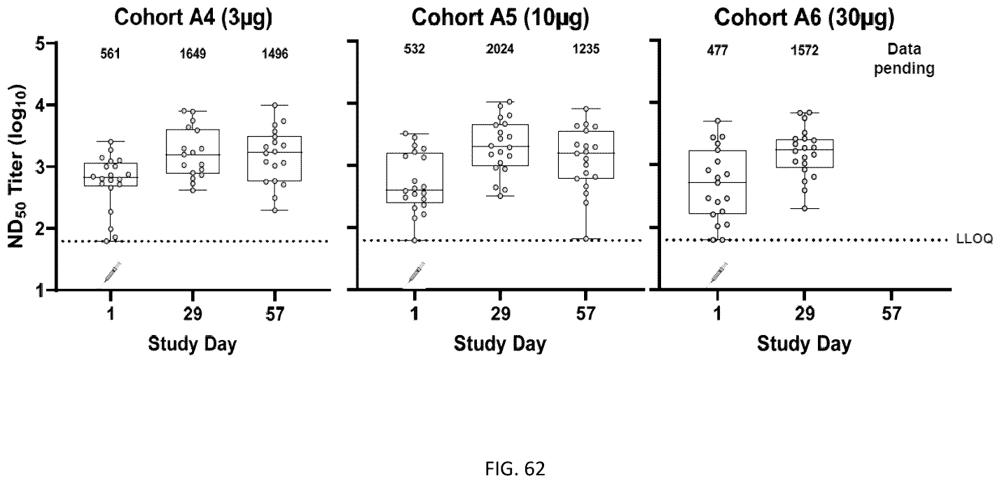

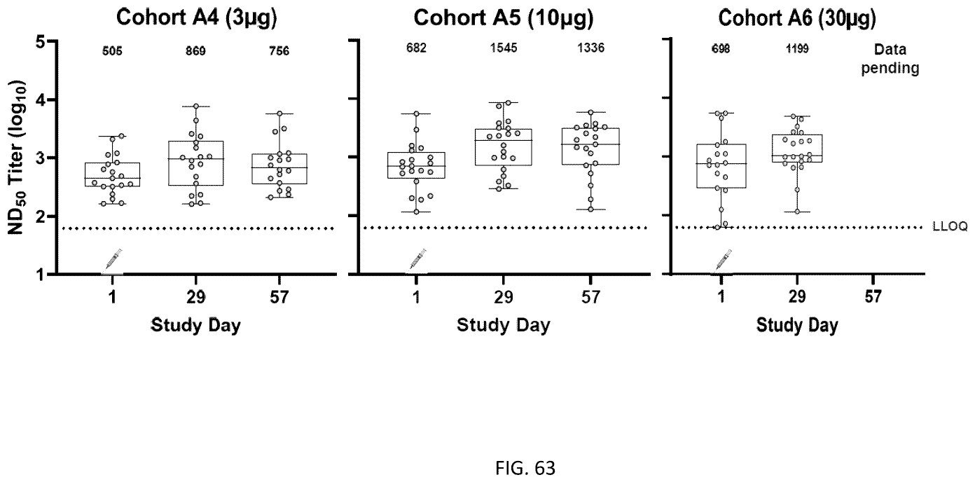

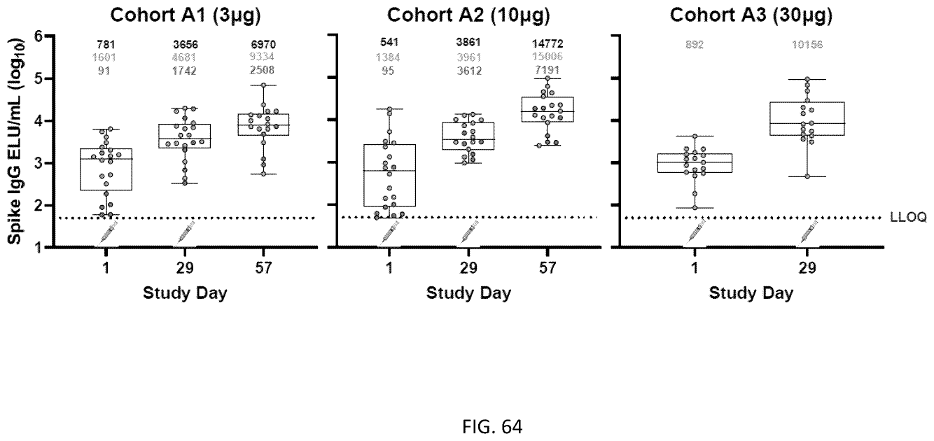

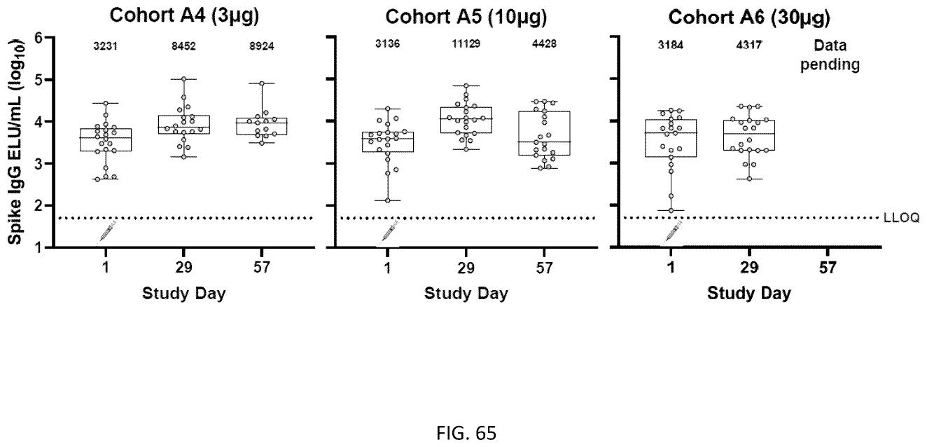

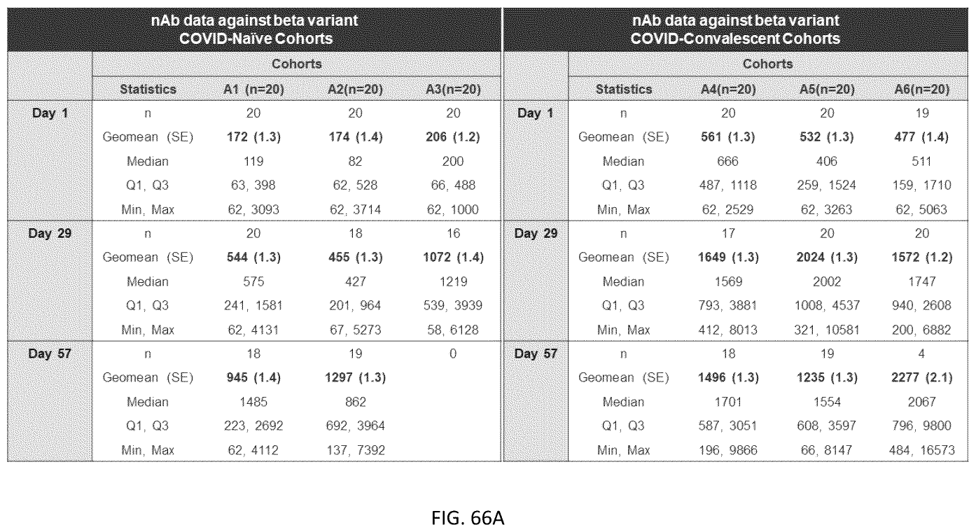

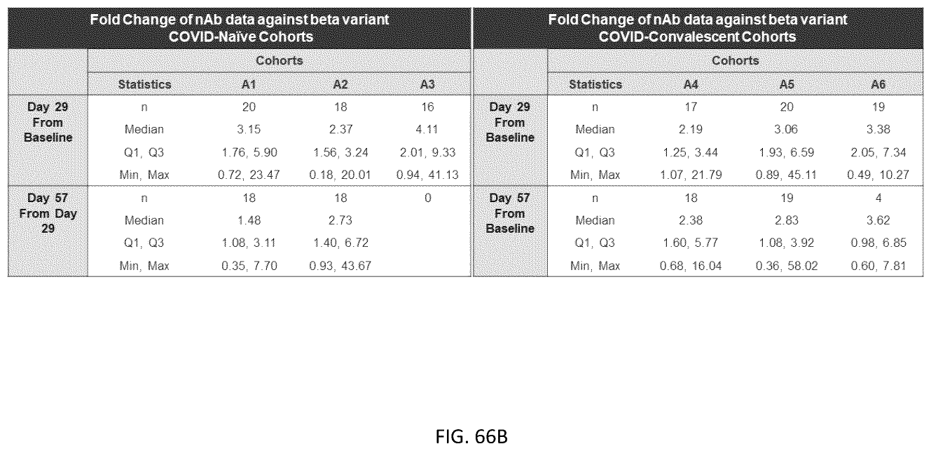

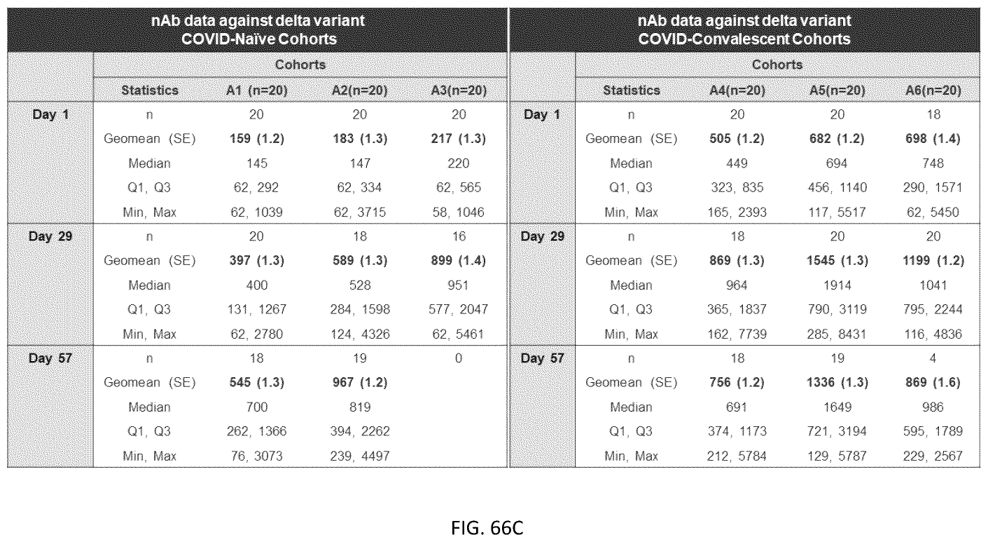

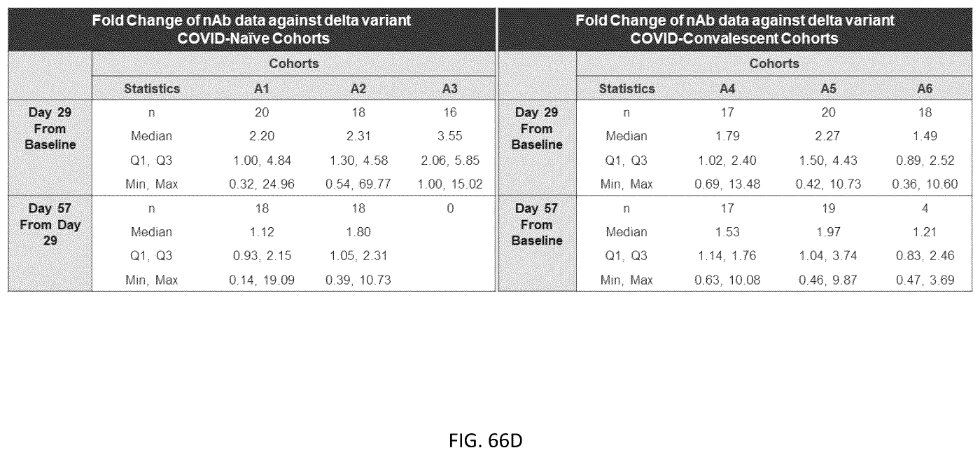

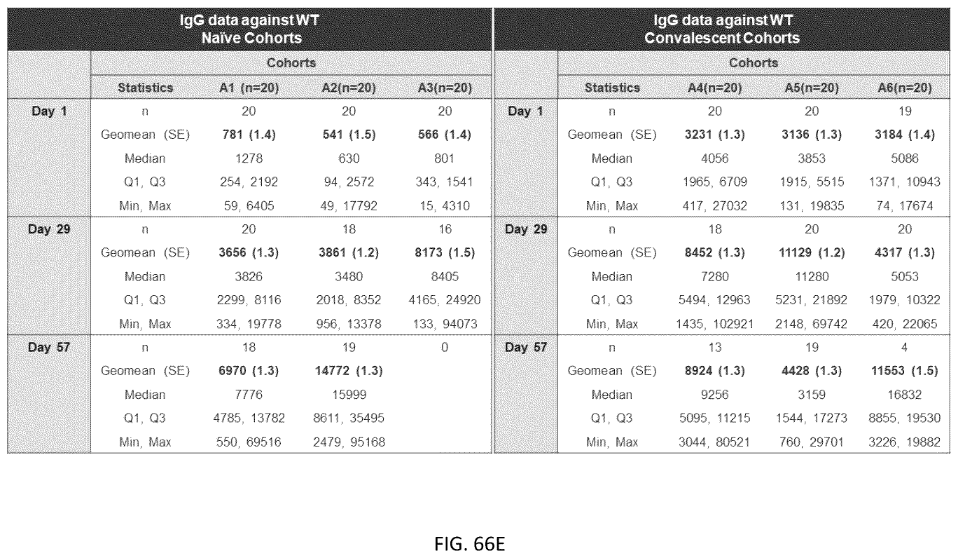

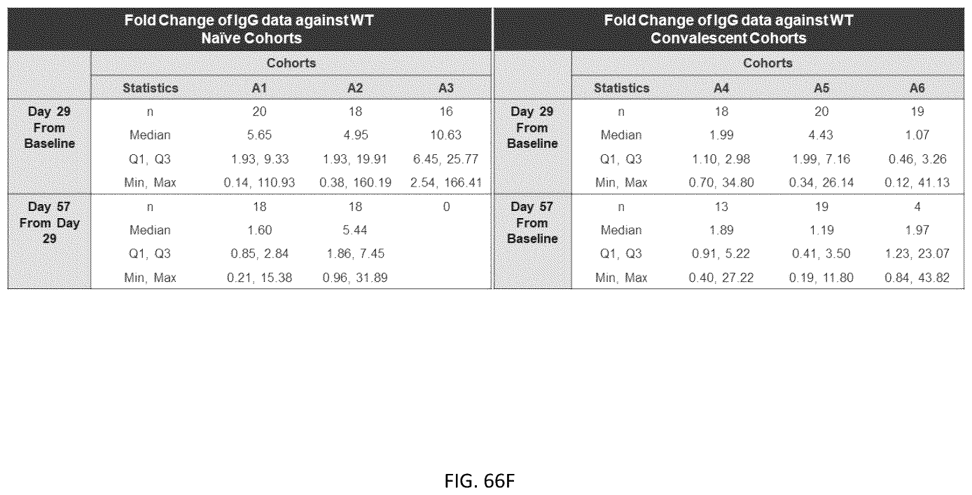

Initial immunogenicity was also assessed for GRT-R914 (TCE9-Spike-beta; SEQ ID NO: 27982). As shown in FIG. 60, COVID-naïve participants demonstrated increased Spike nAb against Beta variant in all samRNA R914 dose levels assessed. As shown in FIG. 61, COVID-naïve participants demonstrated increased Spike nAb against Delta variant in all samRNA R914 dose levels assessed. As shown in FIG. 62, COVID-convalescent participants demonstrated increased Spike nAb against Beta variant in all samRNA R914 dose levels assessed. As shown in FIG. 63, COVID-convalescent participants demonstrated increased Spike nAb against Delta variant in all samRNA R914 dose levels assessed. As shown in FIG. 64, COVID-naïve participants demonstrated an increased trend of IgG against WT variant in all dose levels of 3 μg, 10 μg, and 30 μg samRNA R914. As shown in FIG. 65, COVID-convalescent participants demonstrated increased IgG against WT variant that was maintained until at least Day 57 following administration of 3 μg samRNA (Cohort A4), while the 10 μg samRNA dose (Cohort A5) did not maintain IgG at Day 57 in convalescent subjects. Summaries of statistics are shown for: nAb data against Beta variant (FIG. 66A); fold change against Beta variant (FIG. 66B); nAb data against Delta variant (FIG. 66C); fold change against Delta variant (FIG. 66D); IgG data against wild-type (FIG. 66E); fold change against wild-type (FIG. 66F).

Overall, the results demonstrate for the self-amplifying mRNA SARS-CoV-2 Spike-beta+ TCE-9 vaccine:
All dose levels tested (3 μg, 10 μg, and 30 μg) of GRT-R914 were well tolerated in vaccine-naïve subjects
   Reactogenicity was slightly increased at 30 μg dose level compared to 3 μg and 10 μg
   Mild and moderate AEs were transient in nature and largely resolved within 1-2 days after dosing
   No grade 4 or serious adverse events were observed
All dose levels were immunogenic in all cohorts, both SARS-CoV-2 naïve and convalescent subjects
A dose-response effect was observed, most notably between 3 μg and 10 μg, less clear between 10 and 30 μg
   Strong live virus neutralizing antibody (nAb) titers to beta variant Spike (contained within the vaccine) were broadly elicited—subjects with serological evidence of prior SARS-CoV-2 exposure generally exhibited superior nAb titers versus those with seronegative status
   Strong live virus nAb titers to delta variant virus were also broadly elicited

### Additional Sequences

The sequences referred to below in Tables A-F refer to sequences found in International Application publication number WO2021236854A1 and U.S. Provisional Application No. 63/277,105, each of which is hereby incorporated by reference for all purposes.

### Table A

Refer to SEQ ID NOS. 130-8195 in the Sequence Listing found in International Application publication number WO2021236854A1 and U.S. Provisional Application No. 63/277,105, each of which is hereby incorporated by reference for all purposes. Presented is each candidate MHC Class I epitope encoded by SARS-CoV-2 that was predicted to associate with a given HLA allele with an EDGE score >0.001. Each entry includes the candidate epitope sequence and cognate HLA alleles with a predicted EDGE score greater than 0.001, with each cognate pairing ranked as H (EDGE score >0.1), M (EDGE score between 0.01 and 0.1), and L (EDGE score <0.01). For example, the candidate epitope MESLVPGF (SEQ ID NO: 127) is predicted to pair with HLA-B*18:01, HLA-B*37:01, and HLA-B*07:05 with EDGE scores 0.019, 0.032, and 0.008, respectively. Accordingly, the entry for SEQ ID NO: 130 is “MESLVPGF: B18:01M; B37:01M; B07:05L.”

### Table B

Refer to SEQ ID NOS. 8196-26740 in the Sequence Listing found in International Application publication number WO2021236854A1 and U.S. Provisional Application No. 63/277,105, each of which is hereby incorporated by reference for all purposes. Presented is each candidate MHC Class II epitope encoded by SARS-CoV-2 that was predicted to associate with a given HLA allele with an EDGE score >0.001. Each entry includes the candidate epitope sequence and cognate HLA alleles with a predicted EDGE score greater than 0.001, with each cognate pairing ranked as H (EDGE score >0.1), M (EDGE score between 0.01 and 0.1), and L (EDGE score <0.01). For example, the candidate epitope VELVAELEGI (SEQ ID NO: 128) is predicted to pair with HLA-DQA1*03:02-B1*03:03, HLA-DRB1*11:02, HLA-DQA1*05:05-B1*03:19, and HLA-DPA1*01:03-B1*104:01 with EDGE scores 0.003145, 0.00328, 0.041097, and 0.011613, respectively. Accordingly, the entry for SEQ ID NO: 8219 is “VELVAELEGI: DQA1*03:02-B1*03:03L; DRB1*11:02L; DQA1*05:05-B1*03:19M; DPA1*01:03-B1*104:01M.” Only HLA-DQ and HLA-DP are referred to by their alpha and beta chains. HLA-DR is referred to only by its beta chain as the alpha chain is generally invariable in the human population, with HLA-DR peptide contact regions particularly invariant.

### Table C

Refer to SEQ ID NOS. 26741-27179 in the Sequence Listing found in International Application publication number WO2021236854A1 and U.S. Provisional Application No. 63/277,105, each of which is hereby incorporated by reference for all purposes. Presented are additional MHC Class I epitopes, other than those from the Spike protein, encoded within the optimized cassette that were predicted to associate with a given HLA allele with an EDGE score >0.001. The additional epitopes were determined by calculating population coverage criteria P with all initial epitopes provided by the SARS-CoV-2 Spike protein (SEQ ID NO:59) split into S1 and S2 and applying the optimization algorithms described herein.

### Table D

Refer to SEQ ID NOS. 27180-27495 in the Sequence Listing found in International Application publication number WO2021236854A1 and U.S. Provisional Application No. 63/277,105, each of which is hereby incorporated by reference for all purposes, for SARS-CoV-2 Spike overlapping peptide pools. Each entry includes the stimulatory peptide, SARS-CoV-2 protein source, peptide subpool information, and Table. For example, the stimulatory peptide MFVFLVLLPLVSSQC (SEQ ID NO: 27180) is derived from SARS-CoV-2 Spike protein (Wuhan D614G variant), included in subpool S_Wu_1_2, and found in Table D. Accordingly, the entry for SEQ ID NO. 27180 is “MFVFLVLLPLVSSQC: Spike Wuhan D614G; S_Wu_1_2; Table D”.

### Table E

Refer to SEQ ID NOS. 27496-27603 in the Sequence Listing found in International Application publication number WO2021236854A1 and U.S. Provisional Application No. 63/277,105, each of which is hereby incorporated by reference for all purposes, for TCE5-encoded overlapping peptide pools. Each entry includes the stimulatory peptide, SARS-CoV-2 protein source, peptide subpool information, and Table. For example, the stimulatory peptide LLWPVTLACFVLAAV (SEQ ID NO: 27496) is derived from SARS-CoV-2 Membrane protein, included in subpool OLP_Mem, and found in Table E. Accordingly, the entry for SEQ ID NO. 27496 is “LLWPVTLACFVLAAV: Membrane; OLP_Mem; Table E”.

### Table F

Refer to Sequence Listing, SEQ ID NOS. 27604-27939 in the Sequence Listing found in International Application publication number WO2021236854A1 and U.S. Provisional Application No. 63/277,105, each of which is hereby incorporated by reference for all purposes, for TCE5-encoded minimal epitope peptide pools. Each entry includes the stimulatory peptide, SARS-CoV-2 protein source, peptide subpool information, and Table. For example, the stimulatory peptide ALSKGVHFV (SEQ ID NO: 27604) is derived from SARS-CoV-2 ORF3a protein (frame 52-85), included in subpool Min_validated, and found in Table F. Accordingly, the entry for SEQ ID NO. 27604 is “ALSKGVHFV: ORF3a 52-85; Min_validated; Table F”.

Certain additional sequences for vectors, cassettes, and antibodies referred to herein are described below and referred to by SEQ ID NO.

Tremelimumab VL (SEQ ID NO: 16)
Tremelimumab VH (SEQ ID NO: 17)
Tremelimumab VH CDR1 (SEQ ID NO: 18)
Tremelimumab VH CDR2 (SEQ ID NO: 19)
Tremelimumab VH CDR3 (SEQ ID NO: 20)
Tremelimumab VL CDR1 (SEQ ID NO: 21)
Tremelimumab VL CDR2 (SEQ ID NO: 22)
Tremelimumab VL CDR3 (SEQ ID NO: 23)
Durvalumab (MEDI4736) VL (SEQ ID NO: 24)
MEDI4736 VH (SEQ ID NO: 25)
MEDI4736 VH CDR1 (SEQ ID NO: 26)
MEDI4736 VH CDR2 (SEQ ID NO: 27)
MEDI4736 VH CDR3 (SEQ ID NO: 28)
MEDI4736 VL CDR1 (SEQ ID NO: 29)
MEDI4736 VL CDR2 (SEQ ID NO: 30)
MEDI4736 VL CDR3 (SEQ ID NO: 31)
UbA76-25merPDTT nucleotide (SEQ ID NO: 32)
UbA76-25merPDTT polypeptide (SEQ ID NO: 33)
MAG-25merPDTT nucleotide (SEQ ID NO: 34)
MAG-25merPDTT polypeptide (SEQ ID NO: 35)
Ub7625merPDTT_NoSFL nucleotide (SEQ ID NO: 36)
Ub7625merPDTT_NoSFL polypeptide (SEQ ID NO: 37)
ChAdV68.5WTnt.MAG25mer (SEQ ID NO: 2); AC_000011.1 with E1 (nt 577 to 3403) and E3
(nt 27, 125-31 ,825) sequences deleted; corresponding ATCC VR-594 nucleotides substituted at
five positions; model neoantigen cassette under the control of the CMV promoter/enhancer
inserted in place of deleted E1; SV40 polyA 3′ of cassette
Venezuelan equine encephalitis virus [VEE (SEQ ID NO: 3) GenBank: L01442.2
VEE-MAG25mer (SEQ ID NO: 4); contains MAG-25merPDTT nucleotide (bases 30-1755)
Venezuelan equine encephalitis virus strain TC-83 [TC-83(SEQ ID NO: 5) GenBank: L01443.1
ATAGGCGGCGCATGAGAGAAGCCCAGACCAATTACCTACCCAAAATGGAGAAAGT
TCACGTTGACATCGAGGAAGACAGCCCATTCCTCAGAGCTTTGCAGCGGAGCTTCC
CGCAGTTTGAGGTAGAAGCCAAGCAGGTCACTGATAATGACCATGCTAATGCCAG
AGCGTTTTCGCATCTGGCTTCAAAACTGATCGAAACGGAGGTGGACCCATCCGACA
CGATCCTTGACATTGGAAGTGCGCCCGCCCGCAGAATGTATTCTAAGCACAAGTAT
CATTGTATCTGTCCGATGAGATGTGCGGAAGATCCGGACAGATTGTATAAGTATGC
AACTAAGCTGAAGAAAAACTGTAAGGAAATAACTGATAAGGAATTGGACAAGAAA
ATGAAGGAGCTCGCCGCCGTCATGAGCGACCCTGACCTGGAAACTGAGACTATGTG
CCTCCACGACGACGAGTCGTGTCGCTACGAAGGGCAAGTCGCTGTTTACCAGGATG
TATACGCGGTTGACGGACCGACAAGTCTCTATCACCAAGCCAATAAGGGAGTTAGA
GTCGCCTACTGGATAGGCTTTGACACCACCCCTTTTATGTTTAAGAACTTGGCTGGA
GCATATCCATCATACTCTACCAACTGGGCCGACGAAACCGTGTTAACGGCTCGTAA
CATAGGCCTATGCAGCTCTGACGTTATGGAGCGGTCACGTAGAGGGATGTCCATTC
TTAGAAAGAAGTATTTGAAACCATCCAACAATGTTCTATTCTCTGTTGGCTCGACCA
TCTACCACGAGAAGAGGGACTTACTGAGGAGCTGGCACCTGCCGTCTGTATTTCAC
TTACGTGGCAAGCAAAATTACACATGTCGGTGTGAGACTATAGTTAGTTGCGACGG
GTACGTCGTTAAAAGAATAGCTATCAGTCCAGGCCTGTATGGGAAGCCTTCAGGCT
ATGCTGCTACGATGCACCGCGAGGGATTCTTGTGCTGCAAAGTGACAGACACATTG
AACGGGGAGAGGGTCTCTTTTCCCGTGTGCACGTATGTGCCAGCTACATTGTGTGA
CCAAATGACTGGCATACTGGCAACAGATGTCAGTGCGGACGACGCGCAAAAACTG
CTGGTTGGGCTCAACCAGCGTATAGTCGTCAACGGTCGCACCCAGAGAAACACCAA
TACCATGAAAAATTACCTTTTGCCCGTAGTGGCCCAGGCATTTGCTAGGTGGGCAA
AGGAATATAAGGAAGATCAAGAAGATGAAAGGCCACTAGGACTACGAGATAGAC
AGTTAGTCATGGGGTGTTGTTGGGCTTTTAGAAGGCACAAGATAACATCTATTTAT
AAGCGCCCGGATACCCAAACCATCATCAAAGTGAACAGCGATTTCCACTCATTCGT
GCTGCCCAGGATAGGCAGTAACACATTGGAGATCGGGCTGAGAACAAGAATCAGG
AAAATGTTAGAGGAGCACAAGGAGCCGTCACCTCTCATTACCGCCGAGGACGTAC
AAGAAGCTAAGTGCGCAGCCGATGAGGCTAAGGAGGTGCGTGAAGCCGAGGAGTT
GCGCGCAGCTCTACCACCTTTGGCAGCTGATGTTGAGGAGCCCACTCTGGAAGCCG
ATGTCGACTTGATGTTACAAGAGGCTGGGGCCGGCTCAGTGGAGACACCTCGTGGC
TTGATAAAGGTTACCAGCTACGATGGCGAGGACAAGATCGGCTCTTACGCTGTGCT
TTCTCCGCAGGCTGTACTCAAGAGTGAAAAATTATCTTGCATCCACCCTCTCGCTGA
ACAAGTCATAGTGATAACACACTCTGGCCGAAAAGGGCGTTATGCCGTGGAACCAT
ACCATGGTAAAGTAGTGGTGCCAGAGGGACATGCAATACCCGTCCAGGACTTTCAA
GCTCTGAGTGAAAGTGCCACCATTGTGTACAACGAACGTGAGTTCGTAAACAGGTA
CCTGCACCATATTGCCACACATGGAGGAGCGCTGAACACTGATGAAGAATATTACA
AAACTGTCAAGCCCAGCGAGCACGACGGCGAATACCTGTACGACATCGACAGGAA
ACAGTGCGTCAAGAAAGAACTAGTCACTGGGCTAGGGCTCACAGGCGAGCTGGTG
GATCCTCCCTTCCATGAATTCGCCTACGAGAGTCTGAGAACACGACCAGCCGCTCC
TTACCAAGTACCAACCATAGGGGTGTATGGCGTGCCAGGATCAGGCAAGTCTGGCA
TCATTAAAAGCGCAGTCACCAAAAAAGATCTAGTGGTGAGCGCCAAGAAAGAAAA
CTGTGCAGAAATTATAAGGGACGTCAAGAAAATGAAAGGGCTGGACGTCAATGCC
AGAACTGTGGACTCAGTGCTCTTGAATGGATGCAAACACCCCGTAGAGACCCTGTA
TATTGACGAAGCTTTTGCTTGTCATGCAGGTACTCTCAGAGCGCTCATAGCCATTAT
AAGACCTAAAAAGGCAGTGCTCTGCGGGGATCCCAAACAGTGCGGTTTTTTTAACA
TGATGTGCCTGAAAGTGCATTTTAACCACGAGATTTGCACACAAGTCTTCCACAAA
AGCATCTCTCGCCGTTGCACTAAATCTGTGACTTCGGTCGTCTCAACCTTGTTTTAC
GACAAAAAAATGAGAACGACGAATCCGAAAGAGACTAAGATTGTGATTGACACTA
CCGGCAGTACCAAACCTAAGCAGGACGATCTCATTCTCACTTGTTTCAGAGGGTGG
GTGAAGCAGTTGCAAATAGATTACAAAGGCAACGAAATAATGACGGCAGCTGCCT
CTCAAGGGCTGACCCGTAAAGGTGTGTATGCCGTTCGGTACAAGGTGAATGAAAAT
CCTCTGTACGCACCCACCTCAGAACATGTGAACGTCCTACTGACCCGCACGGAGGA
CCGCATCGTGTGGAAAACACTAGCCGGCGACCCATGGATAAAAACACTGACTGCC
AAGTACCCTGGGAATTTCACTGCCACGATAGAGGAGTGGCAAGCAGAGCATGATG
CCATCATGAGGCACATCTTGGAGAGACCGGACCCTACCGACGTCTTCCAGAATAAG
GCAAACGTGTGTTGGGCCAAGGCTTTAGTGCCGGTGCTGAAGACCGCTGGCATAGA
CATGACCACTGAACAATGGAACACTGTGGATTATTTTGAAACGGACAAAGCTCACT
CAGCAGAGATAGTATTGAACCAACTATGCGTGAGGTTCTTTGGACTCGATCTGGAC
TCCGGTCTATTTTCTGCACCCACTGTTCCGTTATCCATTAGGAATAATCACTGGGAT
AACTCCCCGTCGCCTAACATGTACGGGCTGAATAAAGAAGTGGTCCGTCAGCTCTC
TCGCAGGTACCCACAACTGCCTCGGGCAGTTGCCACTGGAAGAGTCTATGACATGA
ACACTGGTACACTGCGCAATTATGATCCGCGCATAAACCTAGTACCTGTAAACAGA
AGACTGCCTCATGCTTTAGTCCTCCACCATAATGAACACCCACAGAGTGACTTTTCT
TCATTCGTCAGCAAATTGAAGGGCAGAACTGTCCTGGTGGTCGGGGAAAAGTTGTC
CGTCCCAGGCAAAATGGTTGACTGGTTGTCAGACCGGCCTGAGGCTACCTTCAGAG
CTCGGCTGGATTTAGGCATCCCAGGTGATGTGCCCAAATATGACATAATATTTGTT
AATGTGAGGACCCCATATAAATACCATCACTATCAGCAGTGTGAAGACCATGCCAT
TAAGCTTAGCATGTTGACCAAGAAAGCTTGTCTGCATCTGAATCCCGGCGGAACCT
GTGTCAGCATAGGTTATGGTTACGCTGACAGGGCCAGCGAAAGCATCATTGGTGCT
ATAGCGCGGCAGTTCAAGTTTTCCCGGGTATGCAAACCGAAATCCTCACTTGAAGA
GACGGAAGTTCTGTTTGTATTCATTGGGTACGATCGCAAGGCCCGTACGCACAATC
CTTACAAGCTTTCATCAACCTTGACCAACATTTATACAGGTTCCAGACTCCACGAA
GCCGGATGTGCACCCTCATATCATGTGGTGCGAGGGGATATTGCCACGGCCACCGA
AGGAGTGATTATAAATGCTGCTAACAGCAAAGGACAACCTGGCGGAGGGGTGTGC
GGAGCGCTGTATAAGAAATTCCCGGAAAGCTTCGATTTACAGCCGATCGAAGTAGG
AAAAGCGCGACTGGTCAAAGGTGCAGCTAAACATATCATTCATGCCGTAGGACCA
AACTTCAACAAAGTTTCGGAGGTTGAAGGTGACAAACAGTTGGCAGAGGCTTATG
AGTCCATCGCTAAGATTGTCAACGATAACAATTACAAGTCAGTAGCGATTCCACTG
TTGTCCACCGGCATCTTTTCCGGGAACAAAGATCGACTAACCCAATCATTGAACCA
TTTGCTGACAGCTTTAGACACCACTGATGCAGATGTAGCCATATACTGCAGGGACA
AGAAATGGGAAATGACTCTCAAGGAAGCAGTGGCTAGGAGAGAAGCAGTGGAGG
AGATATGCATATCCGACGACTCTTCAGTGACAGAACCTGATGCAGAGCTGGTGAGG
GTGCATCCGAAGAGTTCTTTGGCTGGAAGGAAGGGCTACAGCACAAGCGATGGCA
AAACTTTCTCATATTTGGAAGGGACCAAGTTTCACCAGGCGGCCAAGGATATAGCA
GAAATTAATGCCATGTGGCCCGTTGCAACGGAGGCCAATGAGCAGGTATGCATGTA
TATCCTCGGAGAAAGCATGAGCAGTATTAGGTCGAAATGCCCCGTCGAAGAGTCG
GAAGCCTCCACACCACCTAGCACGCTGCCTTGCTTGTGCATCCATGCCATGACTCC
AGAAAGAGTACAGCGCCTAAAAGCCTCACGTCCAGAACAAATTACTGTGTGCTCAT
CCTTTCCATTGCCGAAGTATAGAATCACTGGTGTGCAGAAGATCCAATGCTCCCAG
CCTATATTGTTCTCACCGAAAGTGCCTGCGTATATTCATCCAAGGAAGTATCTCGTG
GAAACACCACCGGTAGACGAGACTCCGGAGCCATCGGCAGAGAACCAATCCACAG
AGGGGACACCTGAACAACCACCACTTATAACCGAGGATGAGACCAGGACTAGAAC
GCCTGAGCCGATCATCATCGAAGAGGAAGAAGAGGATAGCATAAGTTTGCTGTCA
GATGGCCCGACCCACCAGGTGCTGCAAGTCGAGGCAGACATTCACGGGCCGCCCTC
TGTATCTAGCTCATCCTGGTCCATTCCTCATGCATCCGACTTTGATGTGGACAGTTT
ATCCATACTTGACACCCTGGAGGGAGCTAGCGTGACCAGCGGGGCAACGTCAGCC
GAGACTAACTCTTACTTCGCAAAGAGTATGGAGTTTCTGGCGCGACCGGTGCCTGC
GCCTCGAACAGTATTCAGGAACCCTCCACATCCCGCTCCGCGCACAAGAACACCGT
CACTTGCACCCAGCAGGGCCTGCTCGAGAACCAGCCTAGTTTCCACCCCGCCAGGC
GTGAATAGGGTGATCACTAGAGAGGAGCTCGAGGCGCTTACCCCGTCACGCACTCC
TAGCAGGTCGGTCTCGAGAACCAGCCTGGTCTCCAACCCGCCAGGCGTAAATAGGG
TGATTACAAGAGAGGAGTTTGAGGCGTTCGTAGCACAACAACAATGACGGTTTGAT
GCGGGTGCATACATCTTTTCCTCCGACACCGGTCAAGGGCATTTACAACAAAAATC
AGTAAGGCAAACGGTGCTATCCGAAGTGGTGTTGGAGAGGACCGAATTGGAGATT
TCGTATGCCCCGCGCCTCGACCAAGAAAAAGAAGAATTACTACGCAAGAAATTAC
AGTTAAATCCCACACCTGCTAACAGAAGCAGATACCAGTCCAGGAAGGTGGAGAA
CATGAAAGCCATAACAGCTAGACGTATTCTGCAAGGCCTAGGGCATTATTTGAAGG
CAGAAGGAAAAGTGGAGTGCTACCGAACCCTGCATCCTGTTCCTTTGTATTCATCT
AGTGTGAACCGTGCCTTTTCAAGCCCCAAGGTCGCAGTGGAAGCCTGTAACGCCAT
GTTGAAAGAGAACTTTCCGACTGTGGCTTCTTACTGTATTATTCCAGAGTACGATGC
CTATTTGGACATGGTTGACGGAGCTTCATGCTGCTTAGACACTGCCAGTTTTTGCCC
TGCAAAGCTGCGCAGCTTTCCAAAGAAACACTCCTATTTGGAACCCACAATACGAT
CGGCAGTGCCTTCAGCGATCCAGAACACGCTCCAGAACGTCCTGGCAGCTGCCACA
AAAAGAAATTGCAATGTCACGCAAATGAGAGAATTGCCCGTATTGGATTCGGCGG
CCTTTAATGTGGAATGCTTCAAGAAATATGCGTGTAATAATGAATATTGGGAAACG
TTTAAAGAAAACCCCATCAGGCTTACTGAAGAAAACGTGGTAAATTACATTACCAA
ATTAAAAGGACCAAAAGCTGCTGCTCTTTTTGCGAAGACACATAATTTGAATATGT
TGCAGGACATACCAATGGACAGGTTTGTAATGGACTTAAAGAGAGACGTGAAAGT
GACTCCAGGAACAAAACATACTGAAGAACGGCCCAAGGTACAGGTGATCCAGGCT
GCCGATCCGCTAGCAACAGCGTATCTGTGCGGAATCCACCGAGAGCTGGTTAGGAG
ATTAAATGCGGTCCTGCTTCCGAACATTCATACACTGTTTGATATGTCGGCTGAAGA
CTTTGACGCTATTATAGCCGAGCACTTCCAGCCTGGGGATTGTGTTCTGGAAACTG
ACATCGCGTCGTTTGATAAAAGTGAGGACGACGCCATGGCTCTGACCGCGTTAATG
ATTCTGGAAGACTTAGGTGTGGACGCAGAGCTGTTGACGCTGATTGAGGCGGCTTT
CGGCGAAATTTCATCAATACATTTGCCCACTAAAACTAAATTTAAATTCGGAGCCA
TGATGAAATCTGGAATGTTCCTCACACTGTTTGTGAACACAGTCATTAACATTGTAA
TCGCAAGCAGAGTGTTGAGAGAACGGCTAACCGGATCACCATGTGCAGCATTCATT
GGAGATGACAATATCGTGAAAGGAGTCAAATCGGACAAATTAATGGCAGACAGGT
GCGCCACCTGGTTGAATATGGAAGTCAAGATTATAGATGCTGTGGTGGGCGAGAA
AGCGCCTTATTTCTGTGGAGGGTTTATTTTGTGTGACTCCGTGACCGGCACAGCGTG
CCGTGTGGCAGACCCCCTAAAAAGGCTGTTTAAGCTTGGCAAACCTCTGGCAGCAG
ACGATGAACATGATGATGACAGGAGAAGGGCATTGCATGAAGAGTCAACACGCTG
GAACCGAGTGGGTATTCTTTCAGAGCTGTGCAAGGCAGTAGAATCAAGGTATGAA
ACCGTAGGAACTTCCATCATAGTTATGGCCATGACTACTCTAGCTAGCAGTGTTAA
ATCATTCAGCTACCTGAGAGGGGCCCCTATAACTCTCTACGGCTAACCTGAATGGA
CTACGACATAGTCTAGTCCGCCAAGATGTTCCCGTTCCAGCCAATGTATCCGATGC
AGCCAATGCCCTATCGCAACCCGTTCGCGGCCCCGCGCAGGCCCTGGTTCCCCAGA
ACCGACCCTTTTCTGGCGATGCAGGTGCAGGAATTAACCCGCTCGATGGCTAACCT
GACGTTCAAGCAACGCCGGGACGCGCCACCTGAGGGGCCATCCGCTAAGAAACCG
AAGAAGGAGGCCTCGCAAAAACAGAAAGGGGGAGGCCAAGGGAAGAAGAAGAAG
AACCAAGGGAAGAAGAAGGCTAAGACAGGGCCGCCTAATCCGAAGGCACAGAAT
GGAAACAAGAAGAAGACCAACAAGAAACCAGGCAAGAGACAGCGCATGGTCATG
AAATTGGAATCTGACAAGACGTTCCCAATCATGTTGGAAGGGAAGATAAACGGCT
ACGCTTGTGTGGTCGGAGGGAAGTTATTCAGGCCGATGCATGTGGAAGGCAAGATC
GACAACGACGTTCTGGCCGCGCTTAAGACGAAGAAAGCATCCAAATACGATCTTG
AGTATGCAGATGTGCCACAGAACATGCGGGCCGATACATTCAAATACACCCATGA
GAAACCCCAAGGCTATTACAGCTGGCATCATGGAGCAGTCCAATATGAAAATGGG
CGTTTCACGGTGCCGAAAGGAGTTGGGGCCAAGGGAGACAGCGGACGACCCATTC
TGGATAACCAGGGACGGGTGGTCGCTATTGTGCTGGGAGGTGTGAATGAAGGATCT
AGGACAGCCCTTTCAGTCGTCATGTGGAACGAGAAGGGAGTTACCGTGAAGTATAC
TCCGGAGAACTGCGAGCAATGGTCACTAGTGACCACCATGTGTCTGCTCGCCAATG
TGACGTTCCCATGTGCTCAACCACCAATTTGCTACGACAGAAAACCAGCAGAGACT
TTGGCCATGCTCAGCGTTAACGTTGACAACCCGGGCTACGATGAGCTGCTGGAAGC
AGCTGTTAAGTGCCCCGGAAGGAAAAGGAGATCCACCGAGGAGCTGTTTAATGAG
TATAAGCTAACGCGCCCTTACATGGCCAGATGCATCAGATGTGCAGTTGGGAGCTG
CCATAGTCCAATAGCAATCGAGGCAGTAAAGAGCGACGGGCACGACGGTTATGTT
AGACTTCAGACTTCCTCGCAGTATGGCCTGGATTCCTCCGGCAACTTAAAGGGCAG
GACCATGCGGTATGACATGCACGGGACCATTAAAGAGATACCACTACATCAAGTGT
CACTCTATACATCTCGCCCGTGTCACATTGTGGATGGGCACGGTTATTTCCTGCTTG
CCAGGTGCCCGGCAGGGGACTCCATCACCATGGAATTTAAGAAAGATTCCGTCAGA
CACTCCTGCTCGGTGCCGTATGAAGTGAAATTTAATCCTGTAGGCAGAGAACTCTA
TACTCATCCCCCAGAACACGGAGTAGAGCAAGCGTGCCAAGTCTACGCACATGATG
CACAGAACAGAGGAGCTTATGTCGAGATGCACCTCCCGGGCTCAGAAGTGGACAG
CAGTTTGGTTTCCTTGAGCGGCAGTTCAGTCACCGTGACACCTCCTGATGGGACTA
GCGCCCTGGTGGAATGCGAGTGTGGCGGCACAAAGATCTCCGAGACCATCAACAA
GACAAAACAGTTCAGCCAGTGCACAAAGAAGGAGCAGTGCAGAGCATATCGGCTG
CAGAACGATAAGTGGGTGTATAATTCTGACAAACTGCCCAAAGCAGCGGGAGCCA
CCTTAAAAGGAAAACTGCATGTCCCATTCTTGCTGGCAGACGGCAAATGCACCGTG
CCTCTAGCACCAGAACCTATGATAACCTTCGGTTTCAGATCAGTGTCACTGAAACT
GCACCCTAAGAATCCCACATATCTAATCACCCGCCAACTTGCTGATGAGCCTCACT
ACACGCACGAGCTCATATCTGAACCAGCTGTTAGGAATTTTACCGTCACCGAAAAA
GGGTGGGAGTTTGTATGGGGAAACCACCCGCCGAAAAGGTTTTGGGCACAGGAAA
CAGCACCCGGAAATCCACATGGGCTACCGCACGAGGTGATAACTCATTATTACCAC
AGATACCCTATGTCCACCATCCTGGGTTTGTCAATTTGTGCCGCCATTGCAACCGTT
TCCGTTGCAGCGTCTACCTGGCTGTTTTGCAGATCTAGAGTTGCGTGCCTAACTCCT
TACCGGCTAACACCTAACGCTAGGATACCATTTTGTCTGGCTGTGCTTTGCTGCGCC
CGCACTGCCCGGGCCGAGACCACCTGGGAGTCCTTGGATCACCTATGGAACAATAA
CCAACAGATGTTCTGGATTCAATTGCTGATCCCTCTGGCCGCCTTGATCGTAGTGAC
TCGCCTGCTCAGGTGCGTGTGCTGTGTCGTGCCTTTTTTAGTCATGGCCGGCGCCGC
AGGCGCCGGCGCCTACGAGCACGCGACCACGATGCCGAGCCAAGCGGGAATCTCG
TATAACACTATAGTCAACAGAGCAGGCTACGCACCACTCCCTATCAGCATAACACC
AACAAAGATCAAGCTGATACCTACAGTGAACTTGGAGTACGTCACCTGCCACTACA
AAACAGGAATGGATTCACCAGCCATCAAATGCTGCGGATCTCAGGAATGCACTCCA
ACTTACAGGCCTGATGAACAGTGCAAAGTCTTCACAGGGGTTTACCCGTTCATGTG
GGGTGGTGCATATTGCTTTTGCGACACTGAGAACACCCAAGTCAGCAAGGCCTACG
TAATGAAATCTGACGACTGCCTTGCGGATCATGCTGAAGCATATAAAGCGCACACA
GCCTCAGTGCAGGCGTTCCTCAACATCACAGTGGGAGAACACTCTATTGTGACTAC
CGTGTATGTGAATGGAGAAACTCCTGTGAATTTCAATGGGGTCAAAATAACTGCAG
GTCCGCTTTCCACAGCTTGGACACCCTTTGATCGCAAAATCGTGCAGTATGCCGGG
GAGATCTATAATTATGATTTTCCTGAGTATGGGGCAGGACAACCAGGAGCATTTGG
AGATATACAATCCAGAACAGTCTCAAGCTCTGATCTGTATGCCAATACCAACCTAG
TGCTGCAGAGACCCAAAGCAGGAGCGATCCACGTGCCATACACTCAGGCACCTTCG
GGTTTTGAGCAATGGAAGAAAGATAAAGCTCCATCATTGAAATTTACCGCCCCTTT
CGGATGCGAAATATATACAAACCCCATTCGCGCCGAAAACTGTGCTGTAGGGTCAA
TTCCATTAGCCTTTGACATTCCCGACGCCTTGTTCACCAGGGTGTCAGAAACACCGA
CACTTTCAGCGGCCGAATGCACTCTTAACGAGTGCGTGTATTCTTCCGACTTTGGTG
GGATCGCCACGGTCAAGTACTCGGCCAGCAAGTCAGGCAAGTGCGCAGTCCATGT
GCCATCAGGGACTGCTACCCTAAAAGAAGCAGCAGTCGAGCTAACCGAGCAAGGG
TCGGCGACTATCCATTTCTCGACCGCAAATATCCACCCGGAGTTCAGGCTCCAAAT
ATGCACATCATATGTTACGTGCAAAGGTGATTGTCACCCCCCGAAAGACCATATTG
TGACACACCCTCAGTATCACGCCCAAACATTTACAGCCGCGGTGTCAAAAACCGCG
TGGACGTGGTTAACATCCCTGCTGGGAGGATCAGCCGTAATTATTATAATTGGCTT
GGTGCTGGCTACTATTGTGGCCATGTACGTGCTGACCAACCAGAAACATAATTGAA
TACAGCAGCAATTGGCAAGCTGCTTACATAGAACTCGCGGCGATTGGCATGCCGCC
TTAAAATTTTTATTTTATTTTTCTTTTCTTTTCCGAATCGGATTTTGTTTTTAATATTT
C
VEE Delivery Vector (SEQ ID NO: 6); VEE genome with nucleotides 7544-11175 deleted
[alphavirus structural proteins removed
ATGggcggcgcatgagagaagcccagaccaattacctacccaaaATGGagaaagttcacgttgacatcgaggaagacagcccat
tcctcagagctttgcagcggagcttcccgcagtttgaggtagaagccaagcaggtcactgataatgaccatgctaatgccagagcgttttc
gcatctggcttcaaaactgatcgaaacggaggtggacccatccgacacgatccttgacattggaagtgcgcccgcccgcagaatgtattct
aagcacaagtatcattgtatctgtccgatgagatgtgcggaagatccggacagattgtataagtatgcaactaagctgaagaaaaactgtaa
ggaaataactgataaggaattggacaagaaaatgaaggagctcgccgccgtcatgagcgaccctgacctggaaactgagactatgtgcc
tccacgacgacgagtcgtgtcgctacgaagggcaagtcgctgtttaccaggatgtatacgcggttgacggaccgacaagtctctatcacc
aagccaataagggagttagagtcgcctactggataggctttgacaccaccccttttatgtttaagaacttggctggagcatatccatcatactc
taccaactgggccgacgaaaccgtgttaacggctcgtaacataggcctatgcagctctgacgttatggagcggtcacgtagagggatgtc
cattcttagaaagaagtatttgaaaccatccaacaatgttctattctctgttggctcgaccatctaccacgagaagagggacttactgaggag
ctggcacctgccgtctgtatttcacttacgtggcaagcaaaattacacatgtcggtgtgagactatagttagttgcgacgggtacgtcgttaa
aagaatagctatcagtccaggcctgtatgggaagccttcaggctatgctgctacgatgcaccgcgagggattcttgtgctgcaaagtgaca
gacacattgaacggggagagggtctcttttcccgtgtgcacgtatgtgccagctacattgtgtgaccaaatgactggcatactggcaacag
atgtcagtgcggacgacgcgcaaaaactgctggttgggctcaaccagcgtatagtcgtcaacggtcgcacccagagaaacaccaatacc
atgaaaaattaccttttgcccgtagtggcccaggcatttgctaggtgggcaaaggaatataaggaagatcaagaagatgaaaggccacta
ggactacgagatagacagttagtcatggggtgttgttgggcttttagaaggcacaagataacatctatttataagcgcccggatacccaaac
catcatcaaagtgaacagcgatttccactcattcgtgctgcccaggataggcagtaacacattggagatcgggctgagaacaagaatcag
gaaaatgttagaggagcacaaggagccgtcacctctcattaccgccgaggacgtacaagaagctaagtgcgcagccgatgaggctaag
gaggtgcgtgaagccgaggagttgcgcgcagctctaccacctttggcagctgatgttgaggagcccactctggaagccgatgtcgacttg
atgttacaagaggctggggccggctcagtggagacacctcgtggcttgataaaggttaccagctacgctggcgaggacaagatcggctc
ttacgctgtgctttctccgcaggctgtactcaagagtgaaaaattatcttgcatccaccctctcgctgaacaagtcatagtgataacacactct
ggccgaaaagggcgttatgccgtggaaccataccatggtaaagtagtggtgccagagggacatgcaatacccgtccaggactttcaagc
tctgagtgaaagtgccaccattgtgtacaacgaacgtgagttcgtaaacaggtacctgcaccatattgccacacatggaggagcgctgaac
actgatgaagaatattacaaaactgtcaagcccagcgagcacgacggcgaatacctgtacgacatcgacaggaaacagtgcgtcaagaa
agaactagtcactgggctagggctcacaggcgagctggtggatcctcccttccatgaattcgcctacgagagtctgagaacacgaccagc
cgctccttaccaagtaccaaccataggggtgtatggcgtgccaggatcaggcaagtctggcatcattaaaagcgcagtcaccaaaaaaga
tctagtggtgagcgccaagaaagaaaactgtgcagaaattataagggacgtcaagaaaatgaaagggctggacgtcaatgccagaactg
tggactcagtgctcttgaatggatgcaaacaccccgtagagaccctgtatattgacgaagcttttgcttgtcatgcaggtactctcagagcgc
tcatagccattataagacctaaaaaggcagtgctctgcggggatcccaaacagtgcggtttttttaacatgatgtgcctgaaagtgcattttaa
ccacgagatttgcacacaagtcttccacaaaagcatctctcgccgttgcactaaatctgtgacttcggtcgtctcaaccttgttttacgacaaa
aaaatgagaacgacgaatccgaaagagactaagattgtgattgacactaccggcagtaccaaacctaagcaggacgatctcattctcactt
gtttcagagggtgggtgaagcagttgcaaatagattacaaaggcaacgaaataatgacggcagctgcctctcaagggctgacccgtaaa
ggtgtgtatgccgttcggtacaaggtgaatgaaaatcctctgtacgcacccacctcagaacatgtgaacgtcctactgacccgcacggag
gaccgcatcgtgtggaaaacactagccggcgacccatggataaaaacactgactgccaagtaccctgggaatttcactgccacgataga
ggagtggcaagcagagcatgatgccatcatgaggcacatcttggagagaccggaccctaccgacgtcttccagaataaggcaaacgtgt
gttgggccaaggctttagtgccggtgctgaagaccgctggcatagacatgaccactgaacaatggaacactgtggattattttgaaacgga
caaagctcactcagcagagatagtattgaaccaactatgcgtgaggttctttggactcgatctggactccggtctattttctgcacccactgtt
ccgttatccattaggaataatcactgggataactccccgtcgcctaacatgtacgggctgaataaagaagtggtccgtcagctctctcgcag
gtacccacaactgcctcgggcagttgccactggaagagtctatgacatgaacactggtacactgcgcaattatgatccgcgcataaaccta
gtacctgtaaacagaagactgcctcatgctttagtcctccaccataatgaacacccacagagtgacttttcttcattcgtcagcaaattgaagg
gcagaactgtcctggtggtcggggaaaagttgtccgtcccaggcaaaatggttgactggttgtcagaccggcctgaggctaccttcagag
ctcggctggatttaggcatcccaggtgatgtgcccaaatatgacataatatttgttaatgtgaggaccccatataaataccatcactatcagca
gtgtgaagaccatgccattaagcttagcatgttgaccaagaaagcttgtctgcatctgaatcccggcggaacctgtgtcagcataggttatg
gttacgctgacagggccagcgaaagcatcattggtgctatagcgcggcagttcaagttttcccgggtatgcaaaccgaaatcctcacttga
agagacggaagttctgtttgtattcattgggtacgatcgcaaggcccgtacgcacaatccttacaagctttcatcaaccttgaccaacatttat
acaggttccagactccacgaagccggatgtgcaccctcatatcatgtggtgcgaggggatattgccacggccaccgaaggagtgattata
aatgctgctaacagcaaaggacaacctggcggaggggtgtgcggagcgctgtataagaaattcccggaaagcttcgatttacagccgat
cgaagtaggaaaagcgcgactggtcaaaggtgcagctaaacatatcattcatgccgtaggaccaaacttcaacaaagtttcggaggttga
aggtgacaaacagttggcagaggcttatgagtccatcgctaagattgtcaacgataacaattacaagtcagtagcgattccactgttgtcca
ccggcatcttttccgggaacaaagatcgactaacccaatcattgaaccatttgctgacagctttagacaccactgatgcagatgtagccatat
actgcagggacaagaaatgggaaatgactctcaaggaagcagtggctaggagagaagcagtggaggagatatgcatatccgacgactc
ttcagtgacagaacctgatgcagagctggtgagggtgcatccgaagagttctttggctggaaggaagggctacagcacaagcgatggca
aaactttctcatatttggaagggaccaagtttcaccaggcggccaaggatatagcagaaattaatgccatgtggcccgttgcaacggaggc
caatgagcaggtatgcatgtatatcctcggagaaagcatgagcagtattaggtcgaaatgccccgtcgaagagtcggaagcctccacacc
acctagcacgctgccttgcttgtgcatccatgccatgactccagaaagagtacagcgcctaaaagcctcacgtccagaacaaattactgtgt
gctcatcctttccattgccgaagtatagaatcactggtgtgcagaagatccaatgctcccagcctatattgttctcaccgaaagtgcctgcgta
tattcatccaaggaagtatctcgtggaaacaccaccggtagacgagactccggagccatcggcagagaaccaatccacagaggggaca
cctgaacaaccaccacttataaccgaggatgagaccaggactagaacgcctgagccgatcatcatcgaagaggaagaagaggatagca
taagtttgctgtcagatggcccgacccaccaggtgctgcaagtcgaggcagacattcacgggccgccctctgtatctagctcatcctggtc
cattcctcatgcatccgactttgatgtggacagtttatccatacttgacaccctggagggagctagcgtgaccagcggggcaacgtcagcc
gagactaactcttacttcgcaaagagtatggagtttctggcgcgaccggtgcctgcgcctcgaacagtattcaggaaccctccacatcccg
ctccgcgcacaagaacaccgtcacttgcacccagcagggcctgctcgagaaccagcctagtttccaccccgccaggcgtgaatagggt
gatcactagagaggagctcgaggcgcttaccccgtcacgcactcctagcaggtcggtctcgagaaccagcctggtctccaacccgccag
gcgtaaatagggtgattacaagagaggagtttgaggcgttcgtagcacaacaacaatgacggtttgatgcgggtgcatacatcttttcctcc
gacaccggtcaagggcatttacaacaaaaatcagtaaggcaaacggtgctatccgaagtggtgttggagaggaccgaattggagatttcg
tatgccccgcgcctcgaccaagaaaaagaagaattactacgcaagaaattacagttaaatcccacacctgctaacagaagcagataccag
tccaggaaggtggagaacatgaaagccataacagctagacgtattctgcaaggcctagggcattatttgaaggcagaaggaaaagtgga
gtgctaccgaaccctgcatcctgttcctttgtattcatctagtgtgaaccgtgccttttcaagccccaaggtcgcagtggaagcctgtaacgc
catgttgaaagagaactttccgactgtggcttcttactgtattattccagagtacgatgcctatttggacatggttgacggagcttcatgctgc
ttagacactgccagtttttgccctgcaaagctgcgcagctttccaaagaaacactcctatttggaacccacaatacgatcggcagtgccttcag
cgatccagaacacgctccagaacgtcctggcagctgccacaaaaagaaattgcaatgtcacgcaaatgagagaattgcccgtattggatt
cggcggcctttaatgtggaatgcttcaagaaatatgcgtgtaataatgaatattgggaaacgtttaaagaaaaccccatcaggcttactgaag
aaaacgtggtaaattacattaccaaattaaaaggaccaaaagctgctgctctttttgcgaagacacataatttgaatatgttgcaggacatacc
aatggacaggtttgtaatggacttaaagagagacgtgaaagtgactccaggaacaaaacatactgaagaacggcccaaggtacaggtga
tccaggctgccgatccgctagcaacagcgtatctgtgcggaatccaccgagagctggttaggagattaaatgcggtcctgcttccgaacat
tcatacactgtttgatatgtcggctgaagactttgacgctattatagccgagcacttccagcctggggattgtgttctggaaactgacatcgcg
tcgtttgataaaagtgaggacgacgccatggctctgaccgcgttaatgattctggaagacttaggtgtggacgcagagctgttgacgctgat
tgaggcggctttcggcgaaatttcatcaatacatttgcccactaaaactaaatttaaattcggagccatgatgaaatctggaatgttcctcaca
ctgtttgtgaacacagtcattaacattgtaatcgcaagcagagtgttgagagaacggctaaccggatcaccatgtgcagcattcattggaga
tgacaatatcgtgaaaggagtcaaatcggacaaattaatggcagacaggtgcgccacctggttgaatatggaagtcaagattatagatgct
gtggtgggcgagaaagcgccttatttctgtggagggtttattttgtgtgactccgtgaccggcacagcgtgccgtgtggcagaccccctaa
aaaggctgtttaagcttggcaaacctctggcagcagacgatgaacatgatgatgacaggagaagggcattgcatgaagagtcaacacgc
tggaaccgagtgggtattctttcagagctgtgcaaggcagtagaatcaaggtatgaaaccgtaggaacttccatcatagttatggccatgac
tactctagctagcagtgttaaatcattcagctacctgagaggggcccctataactctctacggcTAAcctgaatggactacgacgtatcac
gcccaaacatttacagccgcggtgtcaaaaaccgcgtggacgtggttaacatccctgctgggaggatcagccgtaattattataattggctt
ggtgctggctactattgtggccatgtacgtgctgaccaaccagaaacataattgaatacagcagcaattggcaagctgcttacatagaactc
gcggcgattggcatgccgccttaaaatttttattttattttttcttttcttttccgaatcggattttgtttttaatatttcAAAAAAAAAAAA
AAAAAAAAAAAAAAAAAAAAAAAAAAAAAAAAAAAAAAAAAAAAAAAAAAAAA
AAAAAAAAAAAAAAA
TC-83 Delivery Vector (SEQ ID NO: 7); TC-83 genome with nucleotides 7544-11175 deleted
[alphavirus structural proteins removed
ATAGGCGGCGCATGAGAGAAGCCCAGACCAATTACCTACCCAAAATGGAGAAAGT
TCACGTTGACATCGAGGAAGACAGCCCATTCCTCAGAGCTTTGCAGCGGAGCTTCC
CGCAGTTTGAGGTAGAAGCCAAGCAGGTCACTGATAATGACCATGCTAATGCCAG
AGCGTTTTCGCATCTGGCTTCAAAACTGATCGAAACGGAGGTGGACCCATCCGACA
CGATCCTTGACATTGGAAGTGCGCCCGCCCGCAGAATGTATTCTAAGCACAAGTAT
CATTGTATCTGTCCGATGAGATGTGCGGAAGATCCGGACAGATTGTATAAGTATGC
AACTAAGCTGAAGAAAAACTGTAAGGAAATAACTGATAAGGAATTGGACAAGAAA
ATGAAGGAGCTCGCCGCCGTCATGAGCGACCCTGACCTGGAAACTGAGACTATGTG
CCTCCACGACGACGAGTCGTGTCGCTACGAAGGGCAAGTCGCTGTTTACCAGGATG
TATACGCGGTTGACGGACCGACAAGTCTCTATCACCAAGCCAATAAGGGAGTTAGA
GTCGCCTACTGGATAGGCTTTGACACCACCCCTTTTATGTTTAAGAACTTGGCTGGA
GCATATCCATCATACTCTACCAACTGGGCCGACGAAACCGTGTTAACGGCTCGTAA
CATAGGCCTATGCAGCTCTGACGTTATGGAGCGGTCACGTAGAGGGATGTCCATTC
TTAGAAAGAAGTATTTGAAACCATCCAACAATGTTCTATTCTCTGTTGGCTCGACCA
TCTACCACGAGAAGAGGGACTTACTGAGGAGCTGGCACCTGCCGTCTGTATTTCAC
TTACGTGGCAAGCAAAATTACACATGTCGGTGTGAGACTATAGTTAGTTGCGACGG
GTACGTCGTTAAAAGAATAGCTATCAGTCCAGGCCTGTATGGGAAGCCTTCAGGCT
ATGCTGCTACGATGCACCGCGAGGGATTCTTGTGCTGCAAAGTGACAGACACATTG
AACGGGGAGAGGGTCTCTTTTCCCGTGTGCACGTATGTGCCAGCTACATTGTGTGA
CCAAATGACTGGCATACTGGCAACAGATGTCAGTGCGGACGACGCGCAAAAACTG
CTGGTTGGGCTCAACCAGCGTATAGTCGTCAACGGTCGCACCCAGAGAAACACCAA
TACCATGAAAAATTACCTTTTGCCCGTAGTGGCCCAGGCATTTGCTAGGTGGGCAA
AGGAATATAAGGAAGATCAAGAAGATGAAAGGCCACTAGGACTACGAGATAGAC
AGTTAGTCATGGGGTGTTGTTGGGCTTTTAGAAGGCACAAGATAACATCTATTTAT
AAGCGCCCGGATACCCAAACCATCATCAAAGTGAACAGCGATTTCCACTCATTCGT
GCTGCCCAGGATAGGCAGTAACACATTGGAGATCGGGCTGAGAACAAGAATCAGG
AAAATGTTAGAGGAGCACAAGGAGCCGTCACCTCTCATTACCGCCGAGGACGTAC
AAGAAGCTAAGTGCGCAGCCGATGAGGCTAAGGAGGTGCGTGAAGCCGAGGAGTT
GCGCGCAGCTCTACCACCTTTGGCAGCTGATGTTGAGGAGCCCACTCTGGAAGCCG
ATGTCGACTTGATGTTACAAGAGGCTGGGGCCGGCTCAGTGGAGACACCTCGTGGC
TTGATAAAGGTTACCAGCTACGATGGCGAGGACAAGATCGGCTCTTACGCTGTGCT
TTCTCCGCAGGCTGTACTCAAGAGTGAAAAATTATCTTGCATCCACCCTCTCGCTGA
ACAAGTCATAGTGATAACACACTCTGGCCGAAAAGGGCGTTATGCCGTGGAACCAT
ACCATGGTAAAGTAGTGGTGCCAGAGGGACATGCAATACCCGTCCAGGACTTTCAA
GCTCTGAGTGAAAGTGCCACCATTGTGTACAACGAACGTGAGTTCGTAAACAGGTA
CCTGCACCATATTGCCACACATGGAGGAGCGCTGAACACTGATGAAGAATATTACA
AAACTGTCAAGCCCAGCGAGCACGACGGCGAATACCTGTACGACATCGACAGGAA
ACAGTGCGTCAAGAAAGAACTAGTCACTGGGCTAGGGCTCACAGGCGAGCTGGTG
GATCCTCCCTTCCATGAATTCGCCTACGAGAGTCTGAGAACACGACCAGCCGCTCC
TTACCAAGTACCAACCATAGGGGTGTATGGCGTGCCAGGATCAGGCAAGTCTGGCA
TCATTAAAAGCGCAGTCACCAAAAAAGATCTAGTGGTGAGCGCCAAGAAAGAAAA
CTGTGCAGAAATTATAAGGGACGTCAAGAAAATGAAAGGGCTGGACGTCAATGCC
AGAACTGTGGACTCAGTGCTCTTGAATGGATGCAAACACCCCGTAGAGACCCTGTA
TATTGACGAAGCTTTTGCTTGTCATGCAGGTACTCTCAGAGCGCTCATAGCCATTAT
AAGACCTAAAAAGGCAGTGCTCTGCGGGGATCCCAAACAGTGCGGTTTTTTTAACA
TGATGTGCCTGAAAGTGCATTTTAACCACGAGATTTGCACACAAGTCTTCCACAAA
AGCATCTCTCGCCGTTGCACTAAATCTGTGACTTCGGTCGTCTCAACCTTGTTTTAC
GACAAAAAAATGAGAACGACGAATCCGAAAGAGACTAAGATTGTGATTGACACTA
CCGGCAGTACCAAACCTAAGCAGGACGATCTCATTCTCACTTGTTTCAGAGGGTGG
GTGAAGCAGTTGCAAATAGATTACAAAGGCAACGAAATAATGACGGCAGCTGCCT
CTCAAGGGCTGACCCGTAAAGGTGTGTATGCCGTTCGGTACAAGGTGAATGAAAAT
CCTCTGTACGCACCCACCTCAGAACATGTGAACGTCCTACTGACCCGCACGGAGGA
CCGCATCGTGTGGAAAACACTAGCCGGCGACCCATGGATAAAAACACTGACTGCC
AAGTACCCTGGGAATTTCACTGCCACGATAGAGGAGTGGCAAGCAGAGCATGATG
CCATCATGAGGCACATCTTGGAGAGACCGGACCCTACCGACGTCTTCCAGAATAAG
GCAAACGTGTGTTGGGCCAAGGCTTTAGTGCCGGTGCTGAAGACCGCTGGCATAGA
CATGACCACTGAACAATGGAACACTGTGGATTATTTTGAAACGGACAAAGCTCACT
CAGCAGAGATAGTATTGAACCAACTATGCGTGAGGTTCTTTGGACTCGATCTGGAC
TCCGGTCTATTTTCTGCACCCACTGTTCCGTTATCCATTAGGAATAATCACTGGGAT
AACTCCCCGTCGCCTAACATGTACGGGCTGAATAAAGAAGTGGTCCGTCAGCTCTC
TCGCAGGTACCCACAACTGCCTCGGGCAGTTGCCACTGGAAGAGTCTATGACATGA
ACACTGGTACACTGCGCAATTATGATCCGCGCATAAACCTAGTACCTGTAAACAGA
AGACTGCCTCATGCTTTAGTCCTCCACCATAATGAACACCCACAGAGTGACTTTTCT
TCATTCGTCAGCAAATTGAAGGGCAGAACTGTCCTGGTGGTCGGGGAAAAGTTGTC
CGTCCCAGGCAAAATGGTTGACTGGTTGTCAGACCGGCCTGAGGCTACCTTCAGAG
CTCGGCTGGATTTAGGCATCCCAGGTGATGTGCCCAAATATGACATAATATTTGTT
AATGTGAGGACCCCATATAAATACCATCACTATCAGCAGTGTGAAGACCATGCCAT
TAAGCTTAGCATGTTGACCAAGAAAGCTTGTCTGCATCTGAATCCCGGCGGAACCT
GTGTCAGCATAGGTTATGGTTACGCTGACAGGGCCAGCGAAAGCATCATTGGTGCT
ATAGCGCGGCAGTTCAAGTTTTCCCGGGTATGCAAACCGAAATCCTCACTTGAAGA
GACGGAAGTTCTGTTTGTATTCATTGGGTACGATCGCAAGGCCCGTACGCACAATC
CTTACAAGCTTTCATCAACCTTGACCAACATTTATACAGGTTCCAGACTCCACGAA
GCCGGATGTGCACCCTCATATCATGTGGTGCGAGGGGATATTGCCACGGCCACCGA
AGGAGTGATTATAAATGCTGCTAACAGCAAAGGACAACCTGGCGGAGGGGTGTGC
GGAGCGCTGTATAAGAAATTCCCGGAAAGCTTCGATTTACAGCCGATCGAAGTAGG
AAAAGCGCGACTGGTCAAAGGTGCAGCTAAACATATCATTCATGCCGTAGGACCA
AACTTCAACAAAGTTTCGGAGGTTGAAGGTGACAAACAGTTGGCAGAGGCTTATG
AGTCCATCGCTAAGATTGTCAACGATAACAATTACAAGTCAGTAGCGATTCCACTG
TTGTCCACCGGCATCTTTTCCGGGAACAAAGATCGACTAACCCAATCATTGAACCA
TTTGCTGACAGCTTTAGACACCACTGATGCAGATGTAGCCATATACTGCAGGGACA
AGAAATGGGAAATGACTCTCAAGGAAGCAGTGGCTAGGAGAGAAGCAGTGGAGG
AGATATGCATATCCGACGACTCTTCAGTGACAGAACCTGATGCAGAGCTGGTGAGG
GTGCATCCGAAGAGTTCTTTGGCTGGAAGGAAGGGCTACAGCACAAGCGATGGCA
AAACTTTCTCATATTTGGAAGGGACCAAGTTTCACCAGGCGGCCAAGGATATAGCA
GAAATTAATGCCATGTGGCCCGTTGCAACGGAGGCCAATGAGCAGGTATGCATGTA
TATCCTCGGAGAAAGCATGAGCAGTATTAGGTCGAAATGCCCCGTCGAAGAGTCG
GAAGCCTCCACACCACCTAGCACGCTGCCTTGCTTGTGCATCCATGCCATGACTCC
AGAAAGAGTACAGCGCCTAAAAGCCTCACGTCCAGAACAAATTACTGTGTGCTCAT
CCTTTCCATTGCCGAAGTATAGAATCACTGGTGTGCAGAAGATCCAATGCTCCCAG
CCTATATTGTTCTCACCGAAAGTGCCTGCGTATATTCATCCAAGGAAGTATCTCGTG
GAAACACCACCGGTAGACGAGACTCCGGAGCCATCGGCAGAGAACCAATCCACAG
AGGGGACACCTGAACAACCACCACTTATAACCGAGGATGAGACCAGGACTAGAAC
GCCTGAGCCGATCATCATCGAAGAGGAAGAAGAGGATAGCATAAGTTTGCTGTCA
GATGGCCCGACCCACCAGGTGCTGCAAGTCGAGGCAGACATTCACGGGCCGCCCTC
TGTATCTAGCTCATCCTGGTCCATTCCTCATGCATCCGACTTTGATGTGGACAGTTT
ATCCATACTTGACACCCTGGAGGGAGCTAGCGTGACCAGCGGGGCAACGTCAGCC
GAGACTAACTCTTACTTCGCAAAGAGTATGGAGTTTCTGGCGCGACCGGTGCCTGC
GCCTCGAACAGTATTCAGGAACCCTCCACATCCCGCTCCGCGCACAAGAACACCGT
CACTTGCACCCAGCAGGGCCTGCTCGAGAACCAGCCTAGTTTCCACCCCGCCAGGC
GTGAATAGGGTGATCACTAGAGAGGAGCTCGAGGCGCTTACCCCGTCACGCACTCC
TAGCAGGTCGGTCTCGAGAACCAGCCTGGTCTCCAACCCGCCAGGCGTAAATAGGG
TGATTACAAGAGAGGAGTTTGAGGCGTTCGTAGCACAACAACAATGACGGTTTGAT
GCGGGTGCATACATCTTTTCCTCCGACACCGGTCAAGGGCATTTACAACAAAAATC
AGTAAGGCAAACGGTGCTATCCGAAGTGGTGTTGGAGAGGACCGAATTGGAGATT
TCGTATGCCCCGCGCCTCGACCAAGAAAAAGAAGAATTACTACGCAAGAAATTAC
AGTTAAATCCCACACCTGCTAACAGAAGCAGATACCAGTCCAGGAAGGTGGAGAA
CATGAAAGCCATAACAGCTAGACGTATTCTGCAAGGCCTAGGGCATTATTTGAAGG
CAGAAGGAAAAGTGGAGTGCTACCGAACCCTGCATCCTGTTCCTTTGTATTCATCT
AGTGTGAACCGTGCCTTTTCAAGCCCCAAGGTCGCAGTGGAAGCCTGTAACGCCAT
GTTGAAAGAGAACTTTCCGACTGTGGCTTCTTACTGTATTATTCCAGAGTACGATGC
CTATTTGGACATGGTTGACGGAGCTTCATGCTGCTTAGACACTGCCAGTTTTTGCCC
TGCAAAGCTGCGCAGCTTTCCAAAGAAACACTCCTATTTGGAACCCACAATACGAT
CGGCAGTGCCTTCAGCGATCCAGAACACGCTCCAGAACGTCCTGGCAGCTGCCACA
AAAAGAAATTGCAATGTCACGCAAATGAGAGAATTGCCCGTATTGGATTCGGCGG
CCTTTAATGTGGAATGCTTCAAGAAATATGCGTGTAATAATGAATATTGGGAAACG
TTTAAAGAAAACCCCATCAGGCTTACTGAAGAAAACGTGGTAAATTACATTACCAA
ATTAAAAGGACCAAAAGCTGCTGCTCTTTTTGCGAAGACACATAATTTGAATATGT
TGCAGGACATACCAATGGACAGGTTTGTAATGGACTTAAAGAGAGACGTGAAAGT
GACTCCAGGAACAAAACATACTGAAGAACGGCCCAAGGTACAGGTGATCCAGGCT
GCCGATCCGCTAGCAACAGCGTATCTGTGCGGAATCCACCGAGAGCTGGTTAGGAG
ATTAAATGCGGTCCTGCTTCCGAACATTCATACACTGTTTGATATGTCGGCTGAAGA
CTTTGACGCTATTATAGCCGAGCACTTCCAGCCTGGGGATTGTGTTCTGGAAACTG
ACATCGCGTCGTTTGATAAAAGTGAGGACGACGCCATGGCTCTGACCGCGTTAATG
ATTCTGGAAGACTTAGGTGTGGACGCAGAGCTGTTGACGCTGATTGAGGCGGCTTT
CGGCGAAATTTCATCAATACATTTGCCCACTAAAACTAAATTTAAATTCGGAGCCA
TGATGAAATCTGGAATGTTCCTCACACTGTTTGTGAACACAGTCATTAACATTGTAA
TCGCAAGCAGAGTGTTGAGAGAACGGCTAACCGGATCACCATGTGCAGCATTCATT
GGAGATGACAATATCGTGAAAGGAGTCAAATCGGACAAATTAATGGCAGACAGGT
GCGCCACCTGGTTGAATATGGAAGTCAAGATTATAGATGCTGTGGTGGGCGAGAA
AGCGCCTTATTTCTGTGGAGGGTTTATTTTGTGTGACTCCGTGACCGGCACAGCGTG
CCGTGTGGCAGACCCCCTAAAAAGGCTGTTTAAGCTTGGCAAACCTCTGGCAGCAG
ACGATGAACATGATGATGACAGGAGAAGGGCATTGCATGAAGAGTCAACACGCTG
GAACCGAGTGGGTATTCTTTCAGAGCTGTGCAAGGCAGTAGAATCAAGGTATGAA
ACCGTAGGAACTTCCATCATAGTTATGGCCATGACTACTCTAGCTAGCAGTGTTAA
ATCATTCAGCTACCTGAGAGGGGCCCCTATAACTCTCTACGGCTAACCTGAATGGA
CTACGACATAGTCTAGTCCGCCAAGATGTTCCCGTTCCAGCCAATGTATCCGATGC
AGCCAATGCCCTATCGCAACCCGTTCGCGGCCCCGCGCAGGCCCTGGTTCCCCAGA
ACCGACCCTTTTCTGGCGATGCAGGTGCAGGAATTAACCCGCTCGATGGCTAACCT
GACGTTCAAGCAACGCCGGGACGCGCCACCTGAGGGGCCATCCGCTAAGAAACCG
AAGAAGGAGGCCTCGCAAAAACAGAAAGGGGGAGGCCAAGGGAAGAAGAAGAAG
AACCAAGGGAAGAAGAAGGCTAAGACAGGGCCGCCTAATCCGAAGGCACAGAAT
GGAAACAAGAAGAAGACCAACAAGAAACCAGGCAAGAGACAGCGCATGGTCATG
AAATTGGAATCTGACAAGACGTTCCCAATCATGTTGGAAGGGAAGATAAACGGCT
ACGCTTGTGTGGTCGGAGGGAAGTTATTCAGGCCGATGCATGTGGAAGGCAAGATC
GACAACGACGTTCTGGCCGCGCTTAAGACGAAGAAAGCATCCAAATACGATCTTG
AGTATGCAGATGTGCCACAGAACATGCGGGCCGATACATTCAAATACACCCATGA
GAAACCCCAAGGCTATTACAGCTGGCATCATGGAGCAGTCCAATATGAAAATGGG
CGTTTCACGGTGCCGAAAGGAGTTGGGGCCAAGGGAGACAGCGGACGACCCATTC
TGGATAACCAGGGACGGGTGGTCGCTATTGTGCTGGGAGGTGTGAATGAAGGATCT
AGGACAGCCCTTTCAGTCGTCATGTGGAACGAGAAGGGAGTTACCGTGAAGTATAC
TCCGGAGAACTGCGAGCAATGGTCACTAGTGACCACCATGTGTCTGCTCGCCAATG
TGACGTTCCCATGTGCTCAACCACCAATTTGCTACGACAGAAAACCAGCAGAGACT
TTGGCCATGCTCAGCGTTAACGTTGACAACCCGGGCTACGATGAGCTGCTGGAAGC
AGCTGTTAAGTGCCCCGGAAGGAAAAGGAGATCCACCGAGGAGCTGTTTAATGAG
TATAAGCTAACGCGCCCTTACATGGCCAGATGCATCAGATGTGCAGTTGGGAGCTG
CCATAGTCCAATAGCAATCGAGGCAGTAAAGAGCGACGGGCACGACGGTTATGTT
AGACTTCAGACTTCCTCGCAGTATGGCCTGGATTCCTCCGGCAACTTAAAGGGCAG
GACCATGCGGTATGACATGCACGGGACCATTAAAGAGATACCACTACATCAAGTGT
CACTCTATACATCTCGCCCGTGTCACATTGTGGATGGGCACGGTTATTTCCTGCTTG
CCAGGTGCCCGGCAGGGGACTCCATCACCATGGAATTTAAGAAAGATTCCGTCAGA
CACTCCTGCTCGGTGCCGTATGAAGTGAAATTTAATCCTGTAGGCAGAGAACTCTA
TACTCATCCCCCAGAACACGGAGTAGAGCAAGCGTGCCAAGTCTACGCACATGATG
CACAGAACAGAGGAGCTTATGTCGAGATGCACCTCCCGGGCTCAGAAGTGGACAG
CAGTTTGGTTTCCTTGAGCGGCAGTTCAGTCACCGTGACACCTCCTGATGGGACTA
GCGCCCTGGTGGAATGCGAGTGTGGCGGCACAAAGATCTCCGAGACCATCAACAA
GACAAAACAGTTCAGCCAGTGCACAAAGAAGGAGCAGTGCAGAGCATATCGGCTG
CAGAACGATAAGTGGGTGTATAATTCTGACAAACTGCCCAAAGCAGCGGGAGCCA
CCTTAAAAGGAAAACTGCATGTCCCATTCTTGCTGGCAGACGGCAAATGCACCGTG
CCTCTAGCACCAGAACCTATGATAACCTTCGGTTTCAGATCAGTGTCACTGAAACT
GCACCCTAAGAATCCCACATATCTAATCACCCGCCAACTTGCTGATGAGCCTCACT
ACACGCACGAGCTCATATCTGAACCAGCTGTTAGGAATTTTACCGTCACCGAAAAA
GGGTGGGAGTTTGTATGGGGAAACCACCCGCCGAAAAGGTTTTGGGCACAGGAAA
CAGCACCCGGAAATCCACATGGGCTACCGCACGAGGTGATAACTCATTATTACCAC
AGATACCCTATGTCCACCATCCTGGGTTTGTCAATTTGTGCCGCCATTGCAACCGTT
TCCGTTGCAGCGTCTACCTGGCTGTTTTGCAGATCTAGAGTTGCGTGCCTAACTCCT
TACCGGCTAACACCTAACGCTAGGATACCATTTTGTCTGGCTGTGCTTTGCTGCGCC
CGCACTGCCCGGGCCGAGACCACCTGGGAGTCCTTGGATCACCTATGGAACAATAA
CCAACAGATGTTCTGGATTCAATTGCTGATCCCTCTGGCCGCCTTGATCGTAGTGAC
TCGCCTGCTCAGGTGCGTGTGCTGTGTCGTGCCTTTTTTAGTCATGGCCGGCGCCGC
AGGCGCCGGCGCCTACGAGCACGCGACCACGATGCCGAGCCAAGCGGGAATCTCG
TATAACACTATAGTCAACAGAGCAGGCTACGCACCACTCCCTATCAGCATAACACC
AACAAAGATCAAGCTGATACCTACAGTGAACTTGGAGTACGTCACCTGCCACTACA
AAACAGGAATGGATTCACCAGCCATCAAATGCTGCGGATCTCAGGAATGCACTCCA
ACTTACAGGCCTGATGAACAGTGCAAAGTCTTCACAGGGGTTTACCCGTTCATGTG
GGGTGGTGCATATTGCTTTTGCGACACTGAGAACACCCAAGTCAGCAAGGCCTACG
TAATGAAATCTGACGACTGCCTTGCGGATCATGCTGAAGCATATAAAGCGCACACA
GCCTCAGTGCAGGCGTTCCTCAACATCACAGTGGGAGAACACTCTATTGTGACTAC
CGTGTATGTGAATGGAGAAACTCCTGTGAATTTCAATGGGGTCAAAATAACTGCAG
GTCCGCTTTCCACAGCTTGGACACCCTTTGATCGCAAAATCGTGCAGTATGCCGGG
GAGATCTATAATTATGATTTTCCTGAGTATGGGGCAGGACAACCAGGAGCATTTGG
AGATATACAATCCAGAACAGTCTCAAGCTCTGATCTGTATGCCAATACCAACCTAG
TGCTGCAGAGACCCAAAGCAGGAGCGATCCACGTGCCATACACTCAGGCACCTTCG
GGTTTTGAGCAATGGAAGAAAGATAAAGCTCCATCATTGAAATTTACCGCCCCTTT
CGGATGCGAAATATATACAAACCCCATTCGCGCCGAAAACTGTGCTGTAGGGTCAA
TTCCATTAGCCTTTGACATTCCCGACGCCTTGTTCACCAGGGTGTCAGAAACACCGA
CACTTTCAGCGGCCGAATGCACTCTTAACGAGTGCGTGTATTCTTCCGACTTTGGTG
GGATCGCCACGGTCAAGTACTCGGCCAGCAAGTCAGGCAAGTGCGCAGTCCATGT
GCCATCAGGGACTGCTACCCTAAAAGAAGCAGCAGTCGAGCTAACCGAGCAAGGG
TCGGCGACTATCCATTTCTCGACCGCAAATATCCACCCGGAGTTCAGGCTCCAAAT
ATGCACATCATATGTTACGTGCAAAGGTGATTGTCACCCCCCGAAAGACCATATTG
TGACACACCCTCAGTATCACGCCCAAACATTTACAGCCGCGGTGTCAAAAACCGCG
TGGACGTGGTTAACATCCCTGCTGGGAGGATCAGCCGTAATTATTATAATTGGCTT
GGTGCTGGCTACTATTGTGGCCATGTACGTGCTGACCAACCAGAAACATAATTGAA
TACAGCAGCAATTGGCAAGCTGCTTACATAGAACTCGCGGCGATTGGCATGCCGCC
TTAAAATTTTTATTTTATTTTTCTTTTCTTTTCCGAATCGGATTTTGTTTTTAATATTT
C
VEE Production Vector (SEQ ID NO: 8); VEE genome with nucleotides 7544-11175 deleted,
plus 5′ T7-promoter, plus 3′ restriction sites
TAATACGACTCACTATAGGATGggcggcgcatgagagaagcccagaccaattacctacccaaaATGGagaaa
gttcacgttgacatcgaggaagacagcccattcctcagagctttgcagcggagcttcccgcagtttgaggtagaagccaagcaggtcact
gataatgaccatgctaatgccagagcgttttcgcatctggcttcaaaactgatcgaaacggaggtggacccatccgacacgatccttgacat
tggaagtgcgcccgcccgcagaatgtattctaagcacaagtatcattgtatctgtccgatgagatgtgcggaagatccggacagattgtata
agtatgcaactaagctgaagaaaaactgtaaggaaataactgataaggaattggacaagaaaatgaaggagctcgccgccgtcatgagc
gaccctgacctggaaactgagactatgtgcctccacgacgacgagtcgtgtcgctacgaagggcaagtcgctgtttaccaggatgtatac
gcggttgacggaccgacaagtctctatcaccaagccaataagggagttagagtcgcctactggataggctttgacaccaccccttttatgttt
aagaacttggctggagcatatccatcatactctaccaactgggccgacgaaaccgtgttaacggctcgtaacataggcctatgcagctctg
acgttatggagcggtcacgtagagggatgtccattcttagaaagaagtatttgaaaccatccaacaatgttctattctctgttggctcgaccat
ctaccacgagaagagggacttactgaggagctggcacctgccgtctgtatttcacttacgtggcaagcaaaattacacatgtcggtgtgag
actatagttagttgcgacgggtacgtcgttaaaagaatagctatcagtccaggcctgtatgggaagccttcaggctatgctgctacgatgca
ccgcgagggattcttgtgctgcaaagtgacagacacattgaacggggagagggtctcttttcccgtgtgcacgtatgtgccagctacattgt
gtgaccaaatgactggcatactggcaacagatgtcagtgcggacgacgcgcaaaaactgctggttgggctcaaccagcgtatagtcgtc
aacggtcgcacccagagaaacaccaataccatgaaaaattaccttttgcccgtagtggcccaggcatttgctaggtgggcaaaggaatat
aaggaagatcaagaagatgaaaggccactaggactacgagatagacagttagtcatggggtgttgttgggcttttagaaggcacaagata
acatctatttataagcgcccggatacccaaaccatcatcaaagtgaacagcgatttccactcattcgtgctgcccaggataggcagtaacac
attggagatcgggctgagaacaagaatcaggaaaatgttagaggagcacaaggagccgtcacctctcattaccgccgaggacgtacaa
gaagctaagtgcgcagccgatgaggctaaggaggtgcgtgaagccgaggagttgcgcgcagctctaccacctttggcagctgatgttga
ggagcccactctggaagccgatgtcgacttgatgttacaagaggctggggccggctcagtggagacacctcgtggcttgataaaggttac
cagctacgctggcgaggacaagatcggctcttacgctgtgctttctccgcaggctgtactcaagagtgaaaaattatcttgcatccaccctct
cgctgaacaagtcatagtgataacacactctggccgaaaagggcgttatgccgtggaaccataccatggtaaagtagtggtgccagagg
gacatgcaatacccgtccaggactttcaagctctgagtgaaagtgccaccattgtgtacaacgaacgtgagttcgtaaacaggtacctgca
ccatattgccacacatggaggagcgctgaacactgatgaagaatattacaaaactgtcaagcccagcgagcacgacggcgaatacctgt
acgacatcgacaggaaacagtgcgtcaagaaagaactagtcactgggctagggctcacaggcgagctggtggatcctcccttccatgaa
ttcgcctacgagagtctgagaacacgaccagccgctccttaccaagtaccaaccataggggtgtatggcgtgccaggatcaggcaagtct
ggcatcattaaaagcgcagtcaccaaaaaagatctagtggtgagcgccaagaaagaaaactgtgcagaaattataagggacgtcaagaa
aatgaaagggctggacgtcaatgccagaactgtggactcagtgctcttgaatggatgcaaacaccccgtagagaccctgtatattgacga
agcttttgcttgtcatgcaggtactctcagagcgctcatagccattataagacctaaaaaggcagtgctctgcggggatcccaaacagtgcg
gtttttttaacatgatgtgcctgaaagtgcattttaaccacgagatttgcacacaagtcttccacaaaagcatctctcgccgttgcactaaatc
tgtgacttcggtcgtctcaaccttgttttacgacaaaaaaatgagaacgacgaatccgaaagagactaagattgtgattgacactaccggcagt
accaaacctaagcaggacgatctcattctcacttgtttcagagggtgggtgaagcagttgcaaatagattacaaaggcaacgaaataatga
cggcagctgcctctcaagggctgacccgtaaaggtgtgtatgccgttcggtacaaggtgaatgaaaatcctctgtacgcacccacctcag
aacatgtgaacgtcctactgacccgcacggaggaccgcatcgtgtggaaaacactagccggcgacccatggataaaaacactgactgc
caagtaccctgggaatttcactgccacgatagaggagtggcaagcagagcatgatgccatcatgaggcacatcttggagagaccggacc
ctaccgacgtcttccagaataaggcaaacgtgtgttgggccaaggctttagtgccggtgctgaagaccgctggcatagacatgaccactg
aacaatggaacactgtggattattttgaaacggacaaagctcactcagcagagatagtattgaaccaactatgcgtgaggttctttggactcg
atctggactccggtctattttctgcacccactgttccgttatccattaggaataatcactgggataactccccgtcgcctaacatgtacgggct
gaataaagaagtggtccgtcagctctctcgcaggtacccacaactgcctcgggcagttgccactggaagagtctatgacatgaacactgg
tacactgcgcaattatgatccgcgcataaacctagtacctgtaaacagaagactgcctcatgctttagtcctccaccataatgaacacccaca
gagtgacttttcttcattcgtcagcaaattgaagggcagaactgtcctggtggtcggggaaaagttgtccgtcccaggcaaaatggttgact
ggttgtcagaccggcctgaggctaccttcagagctcggctggatttaggcatcccaggtgatgtgcccaaatatgacataatatttgttaatg
tgaggaccccatataaataccatcactatcagcagtgtgaagaccatgccattaagcttagcatgttgaccaagaaagcttgtctgcatctga
atcccggcggaacctgtgtcagcataggttatggttacgctgacagggccagcgaaagcatcattggtgctatagcgcggcagttcaagtt
ttcccgggtatgcaaaccgaaatcctcacttgaagagacggaagttctgtttgtattcattgggtacgatcgcaaggcccgtacgcacaatc
cttacaagctttcatcaaccttgaccaacatttatacaggttccagactccacgaagccggatgtgcaccctcatatcatgtggtgcgaggg
gatattgccacggccaccgaaggagtgattataaatgctgctaacagcaaaggacaacctggcggaggggtgtgcggagcgctgtataa
gaaattcccggaaagcttcgatttacagccgatcgaagtaggaaaagcgcgactggtcaaaggtgcagctaaacatatcattcatgccgta
ggaccaaacttcaacaaagtttcggaggttgaaggtgacaaacagttggcagaggcttatgagtccatcgctaagattgtcaacgataaca
attacaagtcagtagcgattccactgttgtccaccggcatcttttccgggaacaaagatcgactaacccaatcattgaaccatttgctgacag
ctttagacaccactgatgcagatgtagccatatactgcagggacaagaaatgggaaatgactctcaaggaagcagtggctaggagagaa
gcagtggaggagatatgcatatccgacgactcttcagtgacagaacctgatgcagagctggtgagggtgcatccgaagagttctttggct
ggaaggaagggctacagcacaagcgatggcaaaactttctcatatttggaagggaccaagtttcaccaggggccaaggatatagcaga
aattaatgccatgtggcccgttgcaacggaggccaatgagcaggtatgcatgtatatcctcggagaaagcatgagcagtattaggtcgaaa
tgccccgtcgaagagtcggaagcctccacaccacctagcacgctgccttgcttgtgcatccatgccatgactccagaaagagtacagcgc
ctaaaagcctcacgtccagaacaaattactgtgtgctcatcctttccattgccgaagtatagaatcactggtgtgcagaagatccaatgctcc
cagcctatattgttctcaccgaaagtgcctgcgtatattcatccaaggaagtatctcgtggaaacaccaccggtagacgagactccggagc
catcggcagagaaccaatccacagaggggacacctgaacaaccaccacttataaccgaggatgagaccaggactagaacgcctgagc
cgatcatcatcgaagaggaagaagaggatagcataagtttgctgtcagatggcccgacccaccaggtgctgcaagtcgaggcagacatt
cacgggccgccctctgtatctagctcatcctggtccattcctcatgcatccgactttgatgtggacagtttatccatacttgacaccctggagg
gagctagcgtgaccagcggggcaacgtcagccgagactaactcttacttcgcaaagagtatggagtttctggcgcgaccggtgcctgcg
cctcgaacagtattcaggaaccctccacatcccgctccgcgcacaagaacaccgtcacttgcacccagcagggcctgctcgagaaccag
cctagtttccaccccgccaggcgtgaatagggtgatcactagagaggagctcgaggcgcttaccccgtcacgcactcctagcaggtcgg
tctcgagaaccagcctggtctccaacccgccaggcgtaaatagggtgattacaagagaggagtttgaggcgttcgtagcacaacaacaat
gacggtttgatgcgggtgcatacatcttttcctccgacaccggtcaagggcatttacaacaaaaatcagtaaggcaaacggtgctatccga
agtggtgttggagaggaccgaattggagatttcgtatgccccgcgcctcgaccaagaaaaagaagaattactacgcaagaaattacagtt
aaatcccacacctgctaacagaagcagataccagtccaggaaggtggagaacatgaaagccataacagctagacgtattctgcaaggcc
tagggcattatttgaaggcagaaggaaaagtggagtgctaccgaaccctgcatcctgttcctttgtattcatctagtgtgaaccgtgccttttc
aagccccaaggtcgcagtggaagcctgtaacgccatgttgaaagagaactttccgactgtggcttcttactgtattattccagagtacgatg
cctatttggacatggttgacggagcttcatgctgcttagacactgccagtttttgccctgcaaagctgcgcagctttccaaagaaacactccta
tttggaacccacaatacgatcggcagtgccttcagcgatccagaacacgctccagaacgtcctggcagctgccacaaaaagaaattgcaa
tgtcacgcaaatgagagaattgcccgtattggattcggcggcctttaatgtggaatgcttcaagaaatatgcgtgtaataatgaatattggga
aacgtttaaagaaaaccccatcaggcttactgaagaaaacgtggtaaattacattaccaaattaaaaggaccaaaagctgctgctctttttgc
gaagacacataatttgaatatgttgcaggacataccaatggacaggtttgtaatggacttaaagagagacgtgaaagtgactccaggaaca
aaacatactgaagaacggcccaaggtacaggtgatccaggctgccgatccgctagcaacagcgtatctgtgcggaatccaccgagagct
ggttaggagattaaatgcggtcctgcttccgaacattcatacactgtttgatatgtcggctgaagactttgacgctattatagccgagcacttc
cagcctggggattgtgttctggaaactgacatcgcgtcgtttgataaaagtgaggacgacgccatggctctgaccgcgttaatgattctgga
agacttaggtgtggacgcagagctgttgacgctgattgaggcggctttcggcgaaatttcatcaatacatttgcccactaaaactaaatttaa
attcggagccatgatgaaatctggaatgttcctcacactgtttgtgaacacagtcattaacattgtaatcgcaagcagagtgttgagagaacg
gctaaccggatcaccatgtgcagcattcattggagatgacaatatcgtgaaaggagtcaaatcggacaaattaatggcagacaggtgcgc
cacctggttgaatatggaagtcaagattatagatgctgtggtgggcgagaaagcgccttatttctgtggagggtttattttgtgtgactccgtg
accggcacagcgtgccgtgtggcagaccccctaaaaaggctgtttaagcttggcaaacctctggcagcagacgatgaacatgatgatga
caggagaagggcattgcatgaagagtcaacacgctggaaccgagtgggtattctttcagagctgtgcaaggcagtagaatcaaggtatg
aaaccgtaggaacttccatcatagttatggccatgactactctagctagcagtgttaaatcattcagctacctgagaggggcccctataactc
tctacggcTAAcctgaatggactacgacgtatcacgcccaaacatttacagccgcggtgtcaaaaaccgcgtggacgtggttaacatcc
ctgctgggaggatcagccgtaattattataattggcttggtgctggctactattgtggccatgtacgtgctgaccaaccagaaacataattga
atacagcagcaattggcaagctgcttacatagaactcgcggcgattggcatgccgccttaaaatttttattttattttttttttcttttccgaa
tcggattttgtttttaatatttcAAAAAAAAAAAAAAAAAAAAAAAAAAAAAAAAAAAAAAAAAAA
AAAAAAAAAAAAAAAAAAAAAAAAAAAAAAAAAAAAA
TC-83 Production Vector (SEQ ID NO: 9); TC-83 genome with nucleotides 7544-11175
deleted, plus 5′ T7-promoter, plus 3′ restriction sites
ATAGGCGGCGCATGAGAGAAGCCCAGACCAATTACCTACCCAAAATGGAGAAAGT
TCACGTTGACATCGAGGAAGACAGCCCATTCCTCAGAGCTTTGCAGCGGAGCTTCC
CGCAGTTTGAGGTAGAAGCCAAGCAGGTCACTGATAATGACCATGCTAATGCCAG
AGCGTTTTCGCATCTGGCTTCAAAACTGATCGAAACGGAGGTGGACCCATCCGACA
CGATCCTTGACATTGGAAGTGCGCCCGCCCGCAGAATGTATTCTAAGCACAAGTAT
CATTGTATCTGTCCGATGAGATGTGCGGAAGATCCGGACAGATTGTATAAGTATGC
AACTAAGCTGAAGAAAAACTGTAAGGAAATAACTGATAAGGAATTGGACAAGAAA
ATGAAGGAGCTCGCCGCCGTCATGAGCGACCCTGACCTGGAAACTGAGACTATGTG
CCTCCACGACGACGAGTCGTGTCGCTACGAAGGGCAAGTCGCTGTTTACCAGGATG
TATACGCGGTTGACGGACCGACAAGTCTCTATCACCAAGCCAATAAGGGAGTTAGA
GTCGCCTACTGGATAGGCTTTGACACCACCCCTTTTATGTTTAAGAACTTGGCTGGA
GCATATCCATCATACTCTACCAACTGGGCCGACGAAACCGTGTTAACGGCTCGTAA
CATAGGCCTATGCAGCTCTGACGTTATGGAGCGGTCACGTAGAGGGATGTCCATTC
TTAGAAAGAAGTATTTGAAACCATCCAACAATGTTCTATTCTCTGTTGGCTCGACCA
TCTACCACGAGAAGAGGGACTTACTGAGGAGCTGGCACCTGCCGTCTGTATTTCAC
TTACGTGGCAAGCAAAATTACACATGTCGGTGTGAGACTATAGTTAGTTGCGACGG
GTACGTCGTTAAAAGAATAGCTATCAGTCCAGGCCTGTATGGGAAGCCTTCAGGCT
ATGCTGCTACGATGCACCGCGAGGGATTCTTGTGCTGCAAAGTGACAGACACATTG
AACGGGGAGAGGGTCTCTTTTCCCGTGTGCACGTATGTGCCAGCTACATTGTGTGA
CCAAATGACTGGCATACTGGCAACAGATGTCAGTGCGGACGACGCGCAAAAACTG
CTGGTTGGGCTCAACCAGCGTATAGTCGTCAACGGTCGCACCCAGAGAAACACCAA
TACCATGAAAAATTACCTTTTGCCCGTAGTGGCCCAGGCATTTGCTAGGTGGGCAA
AGGAATATAAGGAAGATCAAGAAGATGAAAGGCCACTAGGACTACGAGATAGAC
AGTTAGTCATGGGGTGTTGTTGGGCTTTTAGAAGGCACAAGATAACATCTATTTAT
AAGCGCCCGGATACCCAAACCATCATCAAAGTGAACAGCGATTTCCACTCATTCGT
GCTGCCCAGGATAGGCAGTAACACATTGGAGATCGGGCTGAGAACAAGAATCAGG
AAAATGTTAGAGGAGCACAAGGAGCCGTCACCTCTCATTACCGCCGAGGACGTAC
AAGAAGCTAAGTGCGCAGCCGATGAGGCTAAGGAGGTGCGTGAAGCCGAGGAGTT
GCGCGCAGCTCTACCACCTTTGGCAGCTGATGTTGAGGAGCCCACTCTGGAAGCCG
ATGTCGACTTGATGTTACAAGAGGCTGGGGCCGGCTCAGTGGAGACACCTCGTGGC
TTGATAAAGGTTACCAGCTACGATGGCGAGGACAAGATCGGCTCTTACGCTGTGCT
TTCTCCGCAGGCTGTACTCAAGAGTGAAAAATTATCTTGCATCCACCCTCTCGCTGA
ACAAGTCATAGTGATAACACACTCTGGCCGAAAAGGGCGTTATGCCGTGGAACCAT
ACCATGGTAAAGTAGTGGTGCCAGAGGGACATGCAATACCCGTCCAGGACTTTCAA
GCTCTGAGTGAAAGTGCCACCATTGTGTACAACGAACGTGAGTTCGTAAACAGGTA
CCTGCACCATATTGCCACACATGGAGGAGCGCTGAACACTGATGAAGAATATTACA
AAACTGTCAAGCCCAGCGAGCACGACGGCGAATACCTGTACGACATCGACAGGAA
ACAGTGCGTCAAGAAAGAACTAGTCACTGGGCTAGGGCTCACAGGCGAGCTGGTG
GATCCTCCCTTCCATGAATTCGCCTACGAGAGTCTGAGAACACGACCAGCCGCTCC
TTACCAAGTACCAACCATAGGGGTGTATGGCGTGCCAGGATCAGGCAAGTCTGGCA
TCATTAAAAGCGCAGTCACCAAAAAAGATCTAGTGGTGAGCGCCAAGAAAGAAAA
CTGTGCAGAAATTATAAGGGACGTCAAGAAAATGAAAGGGCTGGACGTCAATGCC
AGAACTGTGGACTCAGTGCTCTTGAATGGATGCAAACACCCCGTAGAGACCCTGTA
TATTGACGAAGCTTTTGCTTGTCATGCAGGTACTCTCAGAGCGCTCATAGCCATTAT
AAGACCTAAAAAGGCAGTGCTCTGCGGGGATCCCAAACAGTGCGGTTTTTTTAACA
TGATGTGCCTGAAAGTGCATTTTAACCACGAGATTTGCACACAAGTCTTCCACAAA
AGCATCTCTCGCCGTTGCACTAAATCTGTGACTTCGGTCGTCTCAACCTTGTTTTAC
GACAAAAAAATGAGAACGACGAATCCGAAAGAGACTAAGATTGTGATTGACACTA
CCGGCAGTACCAAACCTAAGCAGGACGATCTCATTCTCACTTGTTTCAGAGGGTGG
GTGAAGCAGTTGCAAATAGATTACAAAGGCAACGAAATAATGACGGCAGCTGCCT
CTCAAGGGCTGACCCGTAAAGGTGTGTATGCCGTTCGGTACAAGGTGAATGAAAAT
CCTCTGTACGCACCCACCTCAGAACATGTGAACGTCCTACTGACCCGCACGGAGGA
CCGCATCGTGTGGAAAACACTAGCCGGCGACCCATGGATAAAAACACTGACTGCC
AAGTACCCTGGGAATTTCACTGCCACGATAGAGGAGTGGCAAGCAGAGCATGATG
CCATCATGAGGCACATCTTGGAGAGACCGGACCCTACCGACGTCTTCCAGAATAAG
GCAAACGTGTGTTGGGCCAAGGCTTTAGTGCCGGTGCTGAAGACCGCTGGCATAGA
CATGACCACTGAACAATGGAACACTGTGGATTATTTTGAAACGGACAAAGCTCACT
CAGCAGAGATAGTATTGAACCAACTATGCGTGAGGTTCTTTGGACTCGATCTGGAC
TCCGGTCTATTTTCTGCACCCACTGTTCCGTTATCCATTAGGAATAATCACTGGGAT
AACTCCCCGTCGCCTAACATGTACGGGCTGAATAAAGAAGTGGTCCGTCAGCTCTC
TCGCAGGTACCCACAACTGCCTCGGGCAGTTGCCACTGGAAGAGTCTATGACATGA
ACACTGGTACACTGCGCAATTATGATCCGCGCATAAACCTAGTACCTGTAAACAGA
AGACTGCCTCATGCTTTAGTCCTCCACCATAATGAACACCCACAGAGTGACTTTTCT
TCATTCGTCAGCAAATTGAAGGGCAGAACTGTCCTGGTGGTCGGGGAAAAGTTGTC
CGTCCCAGGCAAAATGGTTGACTGGTTGTCAGACCGGCCTGAGGCTACCTTCAGAG
CTCGGCTGGATTTAGGCATCCCAGGTGATGTGCCCAAATATGACATAATATTTGTT
AATGTGAGGACCCCATATAAATACCATCACTATCAGCAGTGTGAAGACCATGCCAT
TAAGCTTAGCATGTTGACCAAGAAAGCTTGTCTGCATCTGAATCCCGGCGGAACCT
GTGTCAGCATAGGTTATGGTTACGCTGACAGGGCCAGCGAAAGCATCATTGGTGCT
ATAGCGCGGCAGTTCAAGTTTTCCCGGGTATGCAAACCGAAATCCTCACTTGAAGA
GACGGAAGTTCTGTTTGTATTCATTGGGTACGATCGCAAGGCCCGTACGCACAATC
CTTACAAGCTTTCATCAACCTTGACCAACATTTATACAGGTTCCAGACTCCACGAA
GCCGGATGTGCACCCTCATATCATGTGGTGCGAGGGGATATTGCCACGGCCACCGA
AGGAGTGATTATAAATGCTGCTAACAGCAAAGGACAACCTGGCGGAGGGGTGTGC
GGAGCGCTGTATAAGAAATTCCCGGAAAGCTTCGATTTACAGCCGATCGAAGTAGG
AAAAGCGCGACTGGTCAAAGGTGCAGCTAAACATATCATTCATGCCGTAGGACCA
AACTTCAACAAAGTTTCGGAGGTTGAAGGTGACAAACAGTTGGCAGAGGCTTATG
AGTCCATCGCTAAGATTGTCAACGATAACAATTACAAGTCAGTAGCGATTCCACTG
TTGTCCACCGGCATCTTTTCCGGGAACAAAGATCGACTAACCCAATCATTGAACCA
TTTGCTGACAGCTTTAGACACCACTGATGCAGATGTAGCCATATACTGCAGGGACA
AGAAATGGGAAATGACTCTCAAGGAAGCAGTGGCTAGGAGAGAAGCAGTGGAGG
AGATATGCATATCCGACGACTCTTCAGTGACAGAACCTGATGCAGAGCTGGTGAGG
GTGCATCCGAAGAGTTCTTTGGCTGGAAGGAAGGGCTACAGCACAAGCGATGGCA
AAACTTTCTCATATTTGGAAGGGACCAAGTTTCACCAGGCGGCCAAGGATATAGCA
GAAATTAATGCCATGTGGCCCGTTGCAACGGAGGCCAATGAGCAGGTATGCATGTA
TATCCTCGGAGAAAGCATGAGCAGTATTAGGTCGAAATGCCCCGTCGAAGAGTCG
GAAGCCTCCACACCACCTAGCACGCTGCCTTGCTTGTGCATCCATGCCATGACTCC
AGAAAGAGTACAGCGCCTAAAAGCCTCACGTCCAGAACAAATTACTGTGTGCTCAT
CCTTTCCATTGCCGAAGTATAGAATCACTGGTGTGCAGAAGATCCAATGCTCCCAG
CCTATATTGTTCTCACCGAAAGTGCCTGCGTATATTCATCCAAGGAAGTATCTCGTG
GAAACACCACCGGTAGACGAGACTCCGGAGCCATCGGCAGAGAACCAATCCACAG
AGGGGACACCTGAACAACCACCACTTATAACCGAGGATGAGACCAGGACTAGAAC
GCCTGAGCCGATCATCATCGAAGAGGAAGAAGAGGATAGCATAAGTTTGCTGTCA
GATGGCCCGACCCACCAGGTGCTGCAAGTCGAGGCAGACATTCACGGGCCGCCCTC
TGTATCTAGCTCATCCTGGTCCATTCCTCATGCATCCGACTTTGATGTGGACAGTTT
ATCCATACTTGACACCCTGGAGGGAGCTAGCGTGACCAGCGGGGCAACGTCAGCC
GAGACTAACTCTTACTTCGCAAAGAGTATGGAGTTTCTGGCGCGACCGGTGCCTGC
GCCTCGAACAGTATTCAGGAACCCTCCACATCCCGCTCCGCGCACAAGAACACCGT
CACTTGCACCCAGCAGGGCCTGCTCGAGAACCAGCCTAGTTTCCACCCCGCCAGGC
GTGAATAGGGTGATCACTAGAGAGGAGCTCGAGGCGCTTACCCCGTCACGCACTCC
TAGCAGGTCGGTCTCGAGAACCAGCCTGGTCTCCAACCCGCCAGGCGTAAATAGGG
TGATTACAAGAGAGGAGTTTGAGGCGTTCGTAGCACAACAACAATGACGGTTTGAT
GCGGGTGCATACATCTTTTCCTCCGACACCGGTCAAGGGCATTTACAACAAAAATC
AGTAAGGCAAACGGTGCTATCCGAAGTGGTGTTGGAGAGGACCGAATTGGAGATT
TCGTATGCCCCGCGCCTCGACCAAGAAAAAGAAGAATTACTACGCAAGAAATTAC
AGTTAAATCCCACACCTGCTAACAGAAGCAGATACCAGTCCAGGAAGGTGGAGAA
CATGAAAGCCATAACAGCTAGACGTATTCTGCAAGGCCTAGGGCATTATTTGAAGG
CAGAAGGAAAAGTGGAGTGCTACCGAACCCTGCATCCTGTTCCTTTGTATTCATCT
AGTGTGAACCGTGCCTTTTCAAGCCCCAAGGTCGCAGTGGAAGCCTGTAACGCCAT
GTTGAAAGAGAACTTTCCGACTGTGGCTTCTTACTGTATTATTCCAGAGTACGATGC
CTATTTGGACATGGTTGACGGAGCTTCATGCTGCTTAGACACTGCCAGTTTTTGCCC
TGCAAAGCTGCGCAGCTTTCCAAAGAAACACTCCTATTTGGAACCCACAATACGAT
CGGCAGTGCCTTCAGCGATCCAGAACACGCTCCAGAACGTCCTGGCAGCTGCCACA
AAAAGAAATTGCAATGTCACGCAAATGAGAGAATTGCCCGTATTGGATTCGGCGG
CCTTTAATGTGGAATGCTTCAAGAAATATGCGTGTAATAATGAATATTGGGAAACG
TTTAAAGAAAACCCCATCAGGCTTACTGAAGAAAACGTGGTAAATTACATTACCAA
ATTAAAAGGACCAAAAGCTGCTGCTCTTTTTGCGAAGACACATAATTTGAATATGT
TGCAGGACATACCAATGGACAGGTTTGTAATGGACTTAAAGAGAGACGTGAAAGT
GACTCCAGGAACAAAACATACTGAAGAACGGCCCAAGGTACAGGTGATCCAGGCT
GCCGATCCGCTAGCAACAGCGTATCTGTGCGGAATCCACCGAGAGCTGGTTAGGAG
ATTAAATGCGGTCCTGCTTCCGAACATTCATACACTGTTTGATATGTCGGCTGAAGA
CTTTGACGCTATTATAGCCGAGCACTTCCAGCCTGGGGATTGTGTTCTGGAAACTG
ACATCGCGTCGTTTGATAAAAGTGAGGACGACGCCATGGCTCTGACCGCGTTAATG
ATTCTGGAAGACTTAGGTGTGGACGCAGAGCTGTTGACGCTGATTGAGGCGGCTTT
CGGCGAAATTTCATCAATACATTTGCCCACTAAAACTAAATTTAAATTCGGAGCCA
TGATGAAATCTGGAATGTTCCTCACACTGTTTGTGAACACAGTCATTAACATTGTAA
TCGCAAGCAGAGTGTTGAGAGAACGGCTAACCGGATCACCATGTGCAGCATTCATT
GGAGATGACAATATCGTGAAAGGAGTCAAATCGGACAAATTAATGGCAGACAGGT
GCGCCACCTGGTTGAATATGGAAGTCAAGATTATAGATGCTGTGGTGGGCGAGAA
AGCGCCTTATTTCTGTGGAGGGTTTATTTTGTGTGACTCCGTGACCGGCACAGCGTG
CCGTGTGGCAGACCCCCTAAAAAGGCTGTTTAAGCTTGGCAAACCTCTGGCAGCAG
ACGATGAACATGATGATGACAGGAGAAGGGCATTGCATGAAGAGTCAACACGCTG
GAACCGAGTGGGTATTCTTTCAGAGCTGTGCAAGGCAGTAGAATCAAGGTATGAA
ACCGTAGGAACTTCCATCATAGTTATGGCCATGACTACTCTAGCTAGCAGTGTTAA
ATCATTCAGCTACCTGAGAGGGGCCCCTATAACTCTCTACGGCTAACCTGAATGGA
CTACGACGTATCACGCCCAAACATTTACAGCCGCGGTGTCAAAAACCGCGTGGACG
TGGTTAACATCCCTGCTGGGAGGATCAGCCGTAATTATTATAATTGGCTTGGTGCTG
GCTACTATTGTGGCCATGTACGTGCTGACCAACCAGAAACATAATTGAATACAGCA
GCAATTGGCAAGCTGCTTACATAGAACTCGCGGCGATTGGCATGCCGCCTTAAAAT
TTTTATTTTATTTTTCTTTTCTTTTCCGAATCGGATTTTGTTTTTAATATTTCAAAAAA
AAAAAAAAAAAAAAAAAAAAAAAAAAAAAAAAAAAAAAAAAAAAAAAAAAAAA
AAAAAAAAAAAAAAAAAAAAA
VEE-UbAAY (SEQ ID NO: 14); VEE delivery vector with MHC class I mouse tumor epitopes
SIINFEKL (SEQ ID NO: 129) and AH1-A5 inserted
VEE-Luciferase (SEQ ID NO: 15); VEE delivery vector with luciferase gene inserted at 7545
ubiquitin (SEQ ID NO: 38) >UbG76 0-228
Ubiquitin A76 (SEQ ID NO: 39) >UbA76 0-228
HLA-A2 (MHC class I) signal peptide (SEQ ID NO: 40) >MHC SignalPep 0-78
HLA-A2 (MHC class I) Trans Membrane domain (SEQ ID NO: 41) >HLA A2 TM Domain 0-201
IgK Leader Seq (SEQ ID NO: 42) >IgK Leader Seq 0-60
Human DC-Lamp (SEQ ID NO: 43) >HumanDCLAMP 0-3178
Mouse LAMP1 (SEQ ID NO: 44) >MouseLamp1 0-1858
Human Lamp1 cDNA (SEQ ID NO: 45) >Human Lamp1 0-2339
Tetanus toxoid nulceic acid sequence (SEQ ID NO: 46)
Tetanus toxoid amino acid sequence (SEQ ID NO: 47)
PADRE nulceotide sequence (SEQ ID NO: 48)
PADRE amino acid sequence (SEQ ID NO: 49)
WPRE (SEQ ID NO: 50) >WPRE 0-593
IRES (SEQ ID NO: 51) >eGFP_IRES_SEAP_Insert 1746-2335
GFP (SEQ ID NO: 52)
SEAP (SEQ ID NO: 53)
Firefly Luciferase (SEQ ID NO: 54)
FMDV 2A (SEQ ID NO: 55)
GPGPG linker (SEQ ID NO: 56)
chAd68-Empty-E4deleted (SEQ ID NO: 75): AC_000011.1 with E1 (nt 577 to 3403), E3 (nt
27,125-31, 825), and E4 region (nt 34, 916 to 35,642) sequences deleted and the corresponding
ATCC VR-594 (Independently sequenced Full-Length VR-594 C68 SEQ ID NO: 10)
nucleotides substituted at five positions
NC_045512.2 Severe acute respiratory syndrome coronavirus 2 isolate Wuhan-Hu-1, complete
genome (SEQ ID NO: 76)
SAM in vitro transcription template DNA (SEQ ID NO: 77); VEE genome with nucleotides
7544-11175 deleted, plus minimal 5′ T7-promoter
TAATACGACTCACTATAATGggcggcgcatgagagaagcccagaccaattacctacccaaaATGGagaaagttca
cgttgacatcgaggaagacagcccattcctcagagctttgcagcggagcttcccgcagtttgaggtagaagccaagcaggtcactgataa
tgaccatgctaatgccagagcgttttcgcatctggcttcaaaactgatcgaaacggaggtggacccatccgacacgatccttgacattgga
agtgcgcccgcccgcagaatgtattctaagcacaagtatcattgtatctgtccgatgagatgtgcggaagatccggacagattgtataagta
tgcaactaagctgaagaaaaactgtaaggaaataactgataaggaattggacaagaaaatgaaggagctcgccgccgtcatgagcgacc
ctgacctggaaactgagactatgtgcctccacgacgacgagtcgtgtcgctacgaagggcaagtcgctgtttaccaggatgtatacgcgg
ttgacggaccgacaagtctctatcaccaagccaataagggagttagagtcgcctactggataggctttgacaccaccccttttatgtttaaga
acttggctggagcatatccatcatactctaccaactgggccgacgaaaccgtgttaacggctcgtaacataggcctatgcagctctgacgtt
atggagcggtcacgtagagggatgtccattcttagaaagaagtatttgaaaccatccaacaatgttctattctctgttggctcgaccatctacc
acgagaagagggacttactgaggagctggcacctgccgtctgtatttcacttacgtggcaagcaaaattacacatgtcggtgtgagactat
agttagttgcgacgggtacgtcgttaaaagaatagctatcagtccaggcctgtatgggaagccttcaggctatgctgctacgatgcaccgc
gagggattcttgtgctgcaaagtgacagacacattgaacggggagagggtctcttttcccgtgtgcacgtatgtgccagctacattgtgtga
ccaaatgactggcatactggcaacagatgtcagtgcggacgacgcgcaaaaactgctggttgggctcaaccagcgtatagtcgtcaacg
gtcgcacccagagaaacaccaataccatgaaaaattaccttttgcccgtagtggcccaggcatttgctaggtgggcaaaggaatataagg
aagatcaagaagatgaaaggccactaggactacgagatagacagttagtcatggggtgttgttgggcttttagaaggcacaagataacatc
tatttataagcgcccggatacccaaaccatcatcaaagtgaacagcgatttccactcattcgtgctgcccaggataggcagtaacacattgg
agatcgggctgagaacaagaatcaggaaaatgttagaggagcacaaggagccgtcacctctcattaccgccgaggacgtacaagaagc
taagtgcgcagccgatgaggctaaggaggtgcgtgaagccgaggagttgcgcgcagctctaccacctttggcagctgatgttgaggagc
ccactctggaagccgatgtcgacttgatgttacaagaggctggggccggctcagtggagacacctcgtggcttgataaaggttaccagct
acgctggcgaggacaagatcggctcttacgctgtgctttctccgcaggctgtactcaagagtgaaaaattatcttgcatccaccctctcgctg
aacaagtcatagtgataacacactctggccgaaaagggcgttatgccgtggaaccataccatggtaaagtagtggtgccagagggacat
gcaatacccgtccaggactttcaagctctgagtgaaagtgccaccattgtgtacaacgaacgtgagttcgtaaacaggtacctgcaccatat
tgccacacatggaggagcgctgaacactgatgaagaatattacaaaactgtcaagcccagcgagcacgacggcgaatacctgtacgac
atcgacaggaaacagtgcgtcaagaaagaactagtcactgggctagggctcacaggcgagctggtggatcctcccttccatgaattcgcc
tacgagagtctgagaacacgaccagccgctccttaccaagtaccaaccataggggtgtatggcgtgccaggatcaggcaagtctggcat
cattaaaagcgcagtcaccaaaaaagatctagtggtgagcgccaagaaagaaaactgtgcagaaattataagggacgtcaagaaaatga
aagggctggacgtcaatgccagaactgtggactcagtgctcttgaatggatgcaaacaccccgtagagaccctgtatattgacgaagctttt
gcttgtcatgcaggtactctcagagcgctcatagccattataagacctaaaaaggcagtgctctgcggggatcccaaacagtgcggttttttt
aacatgatgtgcctgaaagtgcattttaaccacgagatttgcacacaagtcttccacaaaagcatctctcgccgttgcactaaatctgtgactt
cggtcgtctcaaccttgttttacgacaaaaaaatgagaacgacgaatccgaaagagactaagattgtgattgacactaccggcagtaccaa
acctaagcaggacgatctcattctcacttgtttcagaggggggtgaagcagttgcaaatagattacaaaggcaacgaaataatgacggca
gctgcctctcaagggctgacccgtaaaggtgtgtatgccgttcggtacaaggtgaatgaaaatcctctgtacgcacccacctcagaacatg
tgaacgtcctactgacccgcacggaggaccgcatcgtgtggaaaacactagccggcgacccatggataaaaacactgactgccaagta
ccctgggaatttcactgccacgatagaggagtggcaagcagagcatgatgccatcatgaggcacatcttggagagaccggaccctaccg
acgtcttccagaataaggcaaacgtgtgttgggccaaggctttagtgccggtgctgaagaccgctggcatagacatgaccactgaacaat
ggaacactgtggattattttgaaacggacaaagctcactcagcagagatagtattgaaccaactatgcgtgaggttctttggactcgatctgg
actccggtctattttctgcacccactgttccgttatccattaggaataatcactgggataactccccgtcgcctaacatgtacgggctgaataa
agaagtggtccgtcagctctctcgcaggtacccacaactgcctcgggcagttgccactggaagagtctatgacatgaacactggtacact
gcgcaattatgatccgcgcataaacctagtacctgtaaacagaagactgcctcatgctttagtcctccaccataatgaacacccacagagtg
acttttcttcattcgtcagcaaattgaagggcagaactgtcctggtggtcggggaaaagttgtccgtcccaggcaaaatggttgactggttgt
cagaccggcctgaggctaccttcagagctcggctggatttaggcatcccaggtgatgtgcccaaatatgacataatatttgttaatgtgagg
accccatataaataccatcactatcagcagtgtgaagaccatgccattaagcttagcatgttgaccaagaaagcttgtctgcatctgaatccc
ggcggaacctgtgtcagcataggttatggttacgctgacagggccagcgaaagcatcattggtgctatagcgcggcagttcaagttttccc
gggtatgcaaaccgaaatcctcacttgaagagacggaagttctgtttgtattcattgggtacgatcgcaaggcccgtacgcacaatccttac
aagctttcatcaaccttgaccaacatttatacaggttccagactccacgaagccggatgtgcaccctcatatcatgtggtgcgaggggatatt
gccacggccaccgaaggagtgattataaatgctgctaacagcaaaggacaacctggcggaggggtgtgcggagcgctgtataagaaat
tcccggaaagcttcgatttacagccgatcgaagtaggaaaagcgcgactggtcaaaggtgcagctaaacatatcattcatgccgtaggac
caaacttcaacaaagtttcggaggttgaaggtgacaaacagttggcagaggcttatgagtccatcgctaagattgtcaacgataacaattac
aagtcagtagcgattccactgttgtccaccggcatcttttccgggaacaaagatcgactaacccaatcattgaaccatttgctgacagcttta
gacaccactgatgcagatgtagccatatactgcagggacaagaaatgggaaatgactctcaaggaagcagtggctaggagagaagcag
tggaggagatatgcatatccgacgactcttcagtgacagaacctgatgcagagctggtgagggtgcatccgaagagttctttggctggaa
ggaagggctacagcacaagcgatggcaaaactttctcatatttggaagggaccaagtttcaccaggggccaaggatatagcagaaatta
atgccatgtggcccgttgcaacggaggccaatgagcaggtatgcatgtatatcctcggagaaagcatgagcagtattaggtcgaaatgcc
ccgtcgaagagtcggaagcctccacaccacctagcacgctgccttgcttgtgcatccatgccatgactccagaaagagtacagcgcctaa
aagcctcacgtccagaacaaattactgtgtgctcatcctttccattgccgaagtatagaatcactggtgtgcagaagatccaatgctcccagc
ctatattgttctcaccgaaagtgcctgcgtatattcatccaaggaagtatctcgtggaaacaccaccggtagacgagactccggagccatcg
gcagagaaccaatccacagaggggacacctgaacaaccaccacttataaccgaggatgagaccaggactagaacgcctgagccgatc
atcatcgaagaggaagaagaggatagcataagtttgctgtcagatggcccgacccaccaggtgctgcaagtcgaggcagacattcacgg
gccgccctctgtatctagctcatcctggtccattcctcatgcatccgactttgatgtggacagtttatccatacttgacaccctggagggagct
agcgtgaccagcggggcaacgtcagccgagactaactcttacttcgcaaagagtatggagtttctggcgcgaccggtgcctgcgcctcg
aacagtattcaggaaccctccacatcccgctccgcgcacaagaacaccgtcacttgcacccagcagggcctgctcgagaaccagcctag
tttccaccccgccaggcgtgaatagggtgatcactagagaggagctcgaggcgcttaccccgtcacgcactcctagcaggtcggtctcg
agaaccagcctggtctccaacccgccaggcgtaaatagggtgattacaagagaggagtttgaggcgttcgtagcacaacaacaatgacg
gtttgatgcgggtgcatacatcttttcctccgacaccggtcaagggcatttacaacaaaaatcagtaaggcaaacggtgctatccgaagtgg
tgttggagaggaccgaattggagatttcgtatgccccgcgcctcgaccaagaaaaagaagaattactacgcaagaaattacagttaaatcc
cacacctgctaacagaagcagataccagtccaggaaggtggagaacatgaaagccataacagctagacgtattctgcaaggcctaggg
cattatttgaaggcagaaggaaaagtggagtgctaccgaaccctgcatcctgttcctttgtattcatctagtgtgaaccgtgccttttcaagcc
ccaaggtcgcagtggaagcctgtaacgccatgttgaaagagaactttccgactgtggcttcttactgtattattccagagtacgatgcctattt
ggacatggttgacggagcttcatgctgcttagacactgccagtttttgccctgcaaagctgcgcagctttccaaagaaacactcctatttgga
acccacaatacgatcggcagtgccttcagcgatccagaacacgctccagaacgtcctggcagctgccacaaaaagaaattgcaatgtca
cgcaaatgagagaattgcccgtattggattcggcggcctttaatgtggaatgcttcaagaaatatgcgtgtaataatgaatattgggaaacgt
ttaaagaaaaccccatcaggcttactgaagaaaacgtggtaaattacattaccaaattaaaaggaccaaaagctgctgctctttttgcgaaga
cacataatttgaatatgttgcaggacataccaatggacaggtttgtaatggacttaaagagagacgtgaaagtgactccaggaacaaaacat
actgaagaacggcccaaggtacaggtgatccaggctgccgatccgctagcaacagcgtatctgtgcggaatccaccgagagctggttag
gagattaaatgcggtcctgcttccgaacattcatacactgtttgatatgtcggctgaagactttgacgctattatagccgagcacttccagcct
ggggattgtgttctggaaactgacatcgcgtcgtttgataaaagtgaggacgacgccatggctctgaccgcgttaatgattctggaagactt
aggtgtggacgcagagctgttgacgctgattgaggcggctttcggcgaaatttcatcaatacatttgcccactaaaactaaatttaaattcgg
agccatgatgaaatctggaatgttcctcacactgtttgtgaacacagtcattaacattgtaatcgcaagcagagtgttgagagaacggctaac
cggatcaccatgtgcagcattcattggagatgacaatatcgtgaaaggagtcaaatcggacaaattaatggcagacaggtgcgccacctg
gttgaatatggaagtcaagattatagatgctgtggtgggcgagaaagcgccttatttctgtggagggtttattttgtgtgactccgtgaccggc
acagcgtgccgtgtggcagaccccctaaaaaggctgtttaagcttggcaaacctctggcagcagacgatgaacatgatgatgacaggag
aagggcattgcatgaagagtcaacacgctggaaccgagtgggtattctttcagagctgtgcaaggcagtagaatcaaggtatgaaaccgt
aggaacttccatcatagttatggccatgactactctagctagcagtgttaaatcattcagctacctgagaggggcccctataactctctacgg
cTAAcctgaatggactacgacgTatcacgcccaaacatttacagccgcggtgtcaaaaaccgcgtggacgtggttaacatccctgctg
ggaggatcagccgtaattattataattggcttggtgctggctactattgtggccatgtacgtgctgaccaaccagaaacataattgaatacag
cagcaattggcaagctgcttacatagaactcgcggcgattggcatgccgccttaaaatttttattttattttttcttttcttttccgaatcgg
attttgtttttaatatttcAAAAAAAAAAAAAAAAAAAAAAAAAAAAAAAAAAAAAAAAAAAAAAA
AAAAAAAAAAAAAAAAAAAAAAAAAAAAAAAAA
SV40 mini-intron (SEQ ID NO: 88)
CMV-CTSpike-FurinMT2P-T2A-E-T2A-M-SV40-CMV-EPE-BGH Nucleotide sequence (SEQ
ID NO: 109)
VOC 202012/01 B.1.1.7 UK Spike variant Amino Acid Sequence (SEQ ID NO: 110)
MFVFLVLLPLVSSQCVNLTTRTQLPPAYTNSFTRGVYYPDKVFRSSVLHSTQDLFLPFFS
NVTWFHAISGTNGTKRFDNPVLPFNDGVYFASTEKSNIIRGWIFGTTLDSKTQSLLIVNN
ATNVVIKVCEFQFCNDPFLGVYHKNNKSWMESEFRVYSSANNCTFEYVSQPFLMDLE
GKQGNFKNLREFVFKNIDGYFKIYSKHTPINLVRDLPQGFSALEPLVDLPIGINITRFQTL
LALHRSYLTPGDSSSGWTAGAAAYYVGYLQPRTFLLKYNENGTITDAVDCALDPLSET
KCTLKSFTVEKGIYQTSNFRVQPTESIVRFPNITNLCPFGEVENATRFASVYAWNRKRIS
NCVADYSVLYNSASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFERDISTEIYQAG
STPCNGVEGFNCYFPLQSYGFQPTYGVGYQPYRVVVLSFELLHAPATVCGPKKSTNLV
KNKCVNFNFNGLTGTGVLTESNKKFLPFQQFGRDIDDTTDAVRDPQTLEILDITPCSFG
GVSVITPGTNTSNQVAVLYQGVNCTEVPVAIHADQLTPTWRVYSTGSNVFQTRAGCLI
GAEHVNNSYECDIPIGAGICASYQTQTNSHGSASSVASQSIIAYTMSLGAENSVAYSNNS
IAIPINFTISVTTEILPVSMTKTSVDCTMYICGDSTECSNLLLQYGSFCTQLNRALTGIAVE
QDKNTQEVFAQVKQIYKTPPIKDFGGFNFSQILPDPSKPSKRSFIEDLLFNKVTLADAGFI
KQYGDCLGDIAARDLICAQKFNGLTVLPPLLTDEMIAQYTSALLAGTITSGWTFGAGA
ALQIPFAMQMAYRFNGIGVTQNVLYENQKLIANQFNSAIGKIQDSLSSTASALGKLQDV
VNQNAQALNTLVKQLSSNFGAISSVLNDILARLDPPEAEVQIDRLITGRLQSLQTYVTQ
QLIRAAEIRASANLAATKMSECVLGQSKRVDFCGKGYHLMSFPQSAPHGVVFLHVTYV
PAQEKNFTTAPAICHDGKAHFPREGVFVSNGTHWFVTQRNFYEPQIITTHNTFVSGNCD
VVIGIVNNTVYDPLQPELDSFKEELDKYFKNHTSPDVDLGDISGINASVVNIQKEIDRLN
EVAKNLNESLIDLQELGKYEQYIKWPWYIWLGFIAGLIAIVMVTIMLCCMTSCCSCLKG
CCSCGSCCKFDEDDSEPVLKGVKLHYT*
CMV-B117Spike-FurinMT2P-SV40-CMV-EPE-BGH (SEQ ID NO: 111)
B.1.351 Spike-FurinMt Amino Acid sequence (South African Spike Variant; Beta variant;
501Y.V2) (SEQ ID NO: 112)
MFVFLVLLPLVSSQCVNFTTRTQLPPAYTNSFTRGVYYPDKVFRSSVLHSTQDLFLPFFS
NVTWFHAIHVSGTNGTKRFANPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLM
DLEGKQGNFKNLREFVFKNIDGYFKIYSKHTPINLVRGLPQGFSALEPLVDLPIGINITRF
QTLHISYLTPGDSSSGWTAGAAAYYVGYLQPRTFLLKYNENGTITDAVDCALDPLSET
KCTLKSFTVEKGIYQTSNFRVQPTESIVRFPNITNLCPFGEVENATRFASVYAWNRKRIS
NCVADYSVLYNSASFSTFKCYGVSPTKLNDLCFTNVYADSFVIRGDEVRQIAPGQTGNI
ADYNYKLPDDFTGCVIAWNSNNLDSKVGGNYNYLYRLFRKSNLKPFERDISTEIYQAG
STPCNGVKGFNCYFPLQSYGFQPTYGVGYQPYRVVVLSFELLHAPATVCGPKKSTNLV
KNKCVNFNFNGLTGTGVLTESNKKFLPFQQFGRDIADTTDAVRDPQTLEILDITPCSFG
GVSVITPGTNTSNQVAVLYQGVNCTEVPVAIHADQLTPTWRVYSTGSNVFQTRAGCLI
GAEHVNNSYECDIPIGAGICASYQTQTNSPGSASSVASQSIIAYTMSLGVENSVAYSNNS
IAIPTNFTISVTTEILPVSMTKTSVDCTMYICGDSTECSNLLLQYGSFCTQLNRALTGIAV
EQDKNTQEVFAQVKQIYKTPPIKDFGGFNFSQILPDPSKPSKRSFIEDLLENKVTLADAG
FIKQYGDCLGDIAARDLICAQKFNGLTVLPPLLTDEMIAQYTSALLAGTITSGWTFGAG
AALQIPFAMQMAYRFNGIGVTQNVLYENQKLIANQFNSAIGKIQDSLSSTASALGKLQD
VVNQNAQALNTLVKQLSSNFGAISSVLNDILSRLDPPEAEVQIDRLITGRLQSLQTYVTQ
QLIRAAEIRASANLAATKMSECVLGQSKRVDFCGKGYHLMSFPQSAPHGVVFLHVTYV
PAQEKNFTTAPAICHDGKAHFPREGVFVSNGTHWFVTQRNFYEPQIITTDNTFVSGNCD
VVIGIVNNTVYDPLQPELDSFKEELDKYFKNHTSPDVDLGDISGINASVVNIQKEIDRLN
EVAKNLNESLIDLQELGKYEQYIKWPWYIWLGFIAGLIAIVMVTIMLCCMTSCCSCLKG
CCSCGSCCKFDEDDSEPVLKGVKLHYT
CMV-B.1351Spike-FurinMt-2P-CMV-TCE5-BGH (SEQ ID NO: 113); B.1.351 Spike sequence
based on CT1 sequence optimization with relevant variant mutations replaced with COOL
codons
SAM-Nuc-TCE11-Spike B.1.1.529 samRNA (SEQ ID NO: 27976); “RT-R918”:
26S sub-genomic promoter 7513-7536; Nucleocapsid 7571-8827; T2A linker 8840-8905;
TCE11 (including GPGPG linkers and both PADRE and Tetanus Toxoid universal MHC class
II epitopes)
8906-9637; 26S sub-genomic promoter 9659-9682; B.1.1.529 (Omicron Spike) 9714-13, 526;
PolyA 13, 813-13, 932; sequence optimized based on CT1 sequence optimization with relevant
variant mutations replaced with COOL codons; Spike modified with mutations 679-682 RRAR
[SEQ ID NO: 125] to GSAS [SEQ ID NO: 126], K983P and V984P
atgggcggcgcatgagagaagcccagaccaattacctacccaaaatggagaaagttcacgttgacatcgaggaagacagcccattcctc
agagctttgcagcggagcttcccgcagtttgaggtagaagccaagcaggtcactgataatgaccatgctaatgccagagcgttttcgcatc
tggcttcaaaactgatcgaaacggaggtggacccatccgacacgatccttgacattggaagtgcgcccgcccgcagaatgtattctaagc
acaagtatcattgtatctgtccgatgagatgtgcggaagatccggacagattgtataagtatgcaactaagctgaagaaaaactgtaaggaa
ataactgataaggaattggacaagaaaatgaaggagctcgccgccgtcatgagcgaccctgacctggaaactgagactatgtgcctcca
cgacgacgagtcgtgtcgctacgaagggcaagtcgctgtttaccaggatgtatacgcggttgacggaccgacaagtctctatcaccaagc
caataagggagttagagtcgcctactggataggctttgacaccaccccttttatgtttaagaacttggctggagcatatccatcatactctacc
aactgggccgacgaaaccgtgttaacggctcgtaacataggcctatgcagctctgacgttatggagcggtcacgtagagggatgtccatt
cttagaaagaagtatttgaaaccatccaacaatgttctattctctgttggctcgaccatctaccacgagaagagggacttactgaggagctgg
cacctgccgtctgtatttcacttacgtggcaagcaaaattacacatgtcggtgtgagactatagttagttgcgacgggtacgtcgttaaaaga
atagctatcagtccaggcctgtatgggaagccttcaggctatgctgctacgatgcaccgcgagggattcttgtgctgcaaagtgacagaca
cattgaacggggagagggtctcttttcccgtgtgcacgtatgtgccagctacattgtgtgaccaaatgactggcatactggcaacagatgtc
agtgcggacgacgcgcaaaaactgctggttgggctcaaccagcgtatagtcgtcaacggtcgcacccagagaaacaccaataccatga
aaaattaccttttgcccgtagtggcccaggcatttgctaggtgggcaaaggaatataaggaagatcaagaagatgaaaggccactaggac
tacgagatagacagttagtcatggggtgttgttgggcttttagaaggcacaagataacatctatttataagcgcccggatacccaaaccatca
tcaaagtgaacagcgatttccactcattcgtgctgcccaggataggcagtaacacattggagatcgggctgagaacaagaatcaggaaaa
tgttagaggagcacaaggagccgtcacctctcattaccgccgaggacgtacaagaagctaagtgcgcagccgatgaggctaaggaggt
gcgtgaagccgaggagttgcgcgcagctctaccacctttggcagctgatgttgaggagcccactctggaagccgatgtcgacttgatgtta
caagaggctggggccggctcagtggagacacctcgtggcttgataaaggttaccagctacgctggcgaggacaagatcggctcttacgc
tgtgctttctccgcaggctgtactcaagagtgaaaaattatcttgcatccaccctctcgctgaacaagtcatagtgataacacactctggccg
aaaagggcgttatgccgtggaaccataccatggtaaagtagtggtgccagagggacatgcaatacccgtccaggactttcaagctctgag
tgaaagtgccaccattgtgtacaacgaacgtgagttcgtaaacaggtacctgcaccatattgccacacatggaggagcgctgaacactgat
gaagaatattacaaaactgtcaagcccagcgagcacgacggcgaatacctgtacgacatcgacaggaaacagtgcgtcaagaaagaac
tagtcactgggctagggctcacaggcgagctggtggatcctcccttccatgaattcgcctacgagagtctgagaacacgaccagccgctc
cttaccaagtaccaaccataggggtgtatggcgtgccaggatcaggcaagtctggcatcattaaaagcgcagtcaccaaaaaagatctag
tggtgagcgccaagaaagaaaactgtgcagaaattataagggacgtcaagaaaatgaaagggctggacgtcaatgccagaactgtgga
ctcagtgctcttgaatggatgcaaacaccccgtagagaccctgtatattgacgaagcttttgcttgtcatgcaggtactctcagagcgctcat
agccattataagacctaaaaaggcagtgctctgcggggatcccaaacagtgcggtttttttaacatgatgtgcctgaaagtgcattttaacca
cgagatttgcacacaagtcttccacaaaagcatctctcgccgttgcactaaatctgtgacttcggtcgtctcaaccttgttttacgacaaaaaa
atgagaacgacgaatccgaaagagactaagattgtgattgacactaccggcagtaccaaacctaagcaggacgatctcattctcacttgttt
cagagggtgggtgaagcagttgcaaatagattacaaaggcaacgaaataatgacggcagctgcctctcaagggctgacccgtaaaggt
gtgtatgccgttcggtacaaggtgaatgaaaatcctctgtacgcacccacctcagaacatgtgaacgtcctactgacccgcacggaggac
cgcatcgtgtggaaaacactagccggcgacccatggataaaaacactgactgccaagtaccctgggaatttcactgccacgatagagga
gtggcaagcagagcatgatgccatcatgaggcacatcttggagagaccggaccctaccgacgtcttccagaataaggcaaacgtgtgtt
gggccaaggctttagtgccggtgctgaagaccgctggcatagacatgaccactgaacaatggaacactgtggattattttgaaacggaca
aagctcactcagcagagatagtattgaaccaactatgcgtgaggttctttggactcgatctggactccggtctattttctgcacccactgttcc
gttatccattaggaataatcactgggataactccccgtcgcctaacatgtacgggctgaataaagaagtggtccgtcagctctctcgcaggt
acccacaactgcctcgggcagttgccactggaagagtctatgacatgaacactggtacactgcgcaattatgatccgcgcataaacctagt
acctgtaaacagaagactgcctcatgctttagtcctccaccataatgaacacccacagagtgacttttcttcattcgtcagcaaattgaaggg
cagaactgtcctggtggtcggggaaaagttgtccgtcccaggcaaaatggttgactggttgtcagaccggcctgaggctaccttcagagct
cggctggatttaggcatcccaggtgatgtgcccaaatatgacataatatttgttaatgtgaggaccccatataaataccatcactatcagcagt
gtgaagaccatgccattaagcttagcatgttgaccaagaaagcttgtctgcatctgaatcccggcggaacctgtgtcagcataggttatggtt
acgctgacagggccagcgaaagcatcattggtgctatagcgcggcagttcaagttttcccgggtatgcaaaccgaaatcctcacttgaag
agacggaagttctgtttgtattcattgggtacgatcgcaaggcccgtacgcacaatccttacaagctttcatcaaccttgaccaacatttatac
aggttccagactccacgaagccggatgtgcaccctcatatcatgtggtgcgaggggatattgccacggccaccgaaggagtgattataaa
tgctgctaacagcaaaggacaacctggcggaggggtgtgcggagcgctgtataagaaattcccggaaagcttcgatttacagccgatcg
aagtaggaaaagcgcgactggtcaaaggtgcagctaaacatatcattcatgccgtaggaccaaacttcaacaaagtttcggaggttgaag
gtgacaaacagttggcagaggcttatgagtccatcgctaagattgtcaacgataacaattacaagtcagtagcgattccactgttgtccacc
ggcatcttttccgggaacaaagatcgactaacccaatcattgaaccatttgctgacagctttagacaccactgatgcagatgtagccatatac
tgcagggacaagaaatgggaaatgactctcaaggaagcagtggctaggagagaagcagtggaggagatatgcatatccgacgactctt
cagtgacagaacctgatgcagagctggtgagggtgcatccgaagagttctttggctggaaggaagggctacagcacaagcgatggcaa
aactttctcatatttggaagggaccaagtttcaccaggcggccaaggatatagcagaaattaatgccatgtggcccgttgcaacggaggcc
aatgagcaggtatgcatgtatatcctcggagaaagcatgagcagtattaggtcgaaatgccccgtcgaagagtcggaagcctccacacca
cctagcacgctgccttgcttgtgcatccatgccatgactccagaaagagtacagcgcctaaaagcctcacgtccagaacaaattactgtgt
gctcatcctttccattgccgaagtatagaatcactggtgtgcagaagatccaatgctcccagcctatattgttctcaccgaaagtgcctgcgta
tattcatccaaggaagtatctcgtggaaacaccaccggtagacgagactccggagccatcggcagagaaccaatccacagaggggaca
cctgaacaaccaccacttataaccgaggatgagaccaggactagaacgcctgagccgatcatcatcgaagaggaagaagaggatagca
taagtttgctgtcagatggcccgacccaccaggtgctgcaagtcgaggcagacattcacgggccgccctctgtatctagctcatcctggtc
cattcctcatgcatccgactttgatgtggacagtttatccatacttgacaccctggagggagctagcgtgaccagcggggcaacgtcagcc
gagactaactcttacttcgcaaagagtatggagtttctggcgcgaccggtgcctgcgcctcgaacagtattcaggaaccctccacatcccg
ctccgcgcacaagaacaccgtcacttgcacccagcagggcctgctcgagaaccagcctagtttccaccccgccaggcgtgaatagggt
gatcactagagaggagctcgaggcgcttaccccgtcacgcactcctagcaggtcggtctcgagaaccagcctggtctccaacccgccag
gcgtaaatagggtgattacaagagaggagtttgaggcgttcgtagcacaacaacaatgacggtttgatgcgggtgcatacatcttttcctcc
gacaccggtcaagggcatttacaacaaaaatcagtaaggcaaacggtgctatccgaagtggtgttggagaggaccgaattggagatttcg
tatgccccgcgcctcgaccaagaaaaagaagaattactacgcaagaaattacagttaaatcccacacctgctaacagaagcagataccag
tccaggaaggtggagaacatgaaagccataacagctagacgtattctgcaaggcctagggcattatttgaaggcagaaggaaaagtgga
gtgctaccgaaccctgcatcctgttcctttgtattcatctagtgtgaaccgtgccttttcaagccccaaggtcgcagtggaagcctgtaacgc
catgttgaaagagaactttccgactgtggcttcttactgtattattccagagtacgatgcctatttggacatggttgacggagcttcatgctgc
ttagacactgccagtttttgccctgcaaagctgcgcagctttccaaagaaacactcctatttggaacccacaatacgatcggcagtgccttcag
cgatccagaacacgctccagaacgtcctggcagctgccacaaaaagaaattgcaatgtcacgcaaatgagagaattgcccgtattggatt
cggcggcctttaatgtggaatgcttcaagaaatatgcgtgtaataatgaatattgggaaacgtttaaagaaaaccccatcaggcttactgaag
aaaacgtggtaaattacattaccaaattaaaaggaccaaaagctgctgctctttttgcgaagacacataatttgaatatgttgcaggacatacc
aatggacaggtttgtaatggacttaaagagagacgtgaaagtgactccaggaacaaaacatactgaagaacggcccaaggtacaggtga
tccaggctgccgatccgctagcaacagcgtatctgtgcggaatccaccgagagctggttaggagattaaatgcggtcctgcttccgaacat
tcatacactgtttgatatgtcggctgaagactttgacgctattatagccgagcacttccagcctggggattgtgttctggaaactgacatcgcg
tcgtttgataaaagtgaggacgacgccatggctctgaccgcgttaatgattctggaagacttaggtgtggacgcagagctgttgacgctgat
tgaggcggctttcggcgaaatttcatcaatacatttgcccactaaaactaaatttaaattcggagccatgatgaaatctggaatgttcctcaca
ctgtttgtgaacacagtcattaacattgtaatcgcaagcagagtgttgagagaacggctaaccggatcaccatgtgcagcattcattggaga
tgacaatatcgtgaaaggagtcaaatcggacaaattaatggcagacaggtgcgccacctggttgaatatggaagtcaagattatagatgct
gtggtgggcgagaaagcgccttatttctgtggagggtttattttgtgtgactccgtgaccggcacagcgtgccgtgtggcagaccccctaa
aaaggctgtttaagcttggcaaacctctggcagcagacgatgaacatgatgatgacaggagaagggcattgcatgaagagtcaacacgc
tggaaccgagtgggtattctttcagagctgtgcaaggcagtagaatcaaggtatgaaaccgtaggaacttccatcatagttatggccatgac
tactctagctagcagtgttaaatcattcagctacctgagaggggcccctataactctctacggctaacctgaatggactacgactctagaata
gtctttaattaagccaccATGTCAGATAATGGGCCTCAGAACCAGCGGAATGCCCCCAGGA
TCACGTTCGGTGGGCCTTCAGATTCAACTGGAAGCAATCAGAACGGGGAAAG
ATCCGGTGCAAGGTCCAAGCAACGCCGACCCCAGGGATTGCCTAATAATACA
GCAAGTTGGTTTACTGCACTGACCCAACACGGGAAAGAGGACCTTAAATTTCC
GCGGGGGCAAGGAGTACCTATAAACACTAATAGCAGCCCCGATGACCAAATT
GGGTACTACCGCAGAGCAACCCGGCGAATCAGAGGGGGTGACGGTAAGATGA
AGGACCTCTCCCCCAGGTGGTACTTCTACTATTTGGGAACGGGGCCAGAAGCC
GGACTGCCCTACGGCGCAAAAAAGACGGTATAATATGGGTCGCCACTGAGGG
CGCTCTGAATACGCCAAAAGATCACATTGGTACTAGAAACCCGGCGAACAATG
CGGCAATTGTGCTTCAGCTCCCTCAAGGAACAACGTTGCCAAAAGGATTTTAC
GCAGAGGGATCACGGGGGGGTTCCCAGGCTAGTTCTCGCAGTTCCTCAAGGA
GTCGAAATTCCAGTAGGAACTCAACCCCAGGCTCAAGCCGCGGGACTTCACCT
GCAAGAATGGCCGGTAACGGGGGAGACGCTGCTTTGGCTCTTCTTCTCCTTGA
TCGCCTTAATCAGCTTGAGTCTAAGATGTCTGGTAAAGGCCAACAGCAACAAG
GACAGACGGTAACGAAAAAATCCGCCGCCGAGGCCTCTAAAAAACCGAGACA
AAAACGGACTGCTACGAAAGCGTATAACGTCACTCAGGCTTTTGGTAGGCGG
GGCCCGGAACAAACCCAGGGTAATTTTGGCGATCAGGAACTGATTAGACAAG
GCACCGACTACAAGCACTGGCCTCAGATCGCGCAGTTTGCACCCTCCGCTTCA
GCGTTCTTCGGGATGTCACGCATCGGGATGGAGGTTACACCGAGTGGAACAT
GGCTGACATATACTGGTGCAATcAAGCTGGACGACAAGGATCCCAACTTCAAA
GATCAAGTTATACTCCTCAACAAACATATCGATGCGTATAAGACATTCCCCCC
GACGGAACCCAAAAAAGATAAAAAAAAGAAGGCCGACGAGACCCAAGCCCTC
CCACAACGACAAAAGAAACAACAAACAGTAACACTGCTCCCAGCCGCAGACTT
GGATGATTTCTCCAAACAGTTGCAGCAATCTATGAGTTCAGCCGATAGTACTC
AGGCCGGCGGTTCTGGAGGTGTCCGTGCTGAAGGACGCGGGTCCCTTCTCACCTGC
GGCGACGTTGAGGAAAACCCTGGCCCTATGGCTGGCAACGTAGCTTTCCAGACCGT
GAAGCCGGGAAATTTCAACAAGGACTTCTACGACTTCGCCGTCAGCAAGGGATTCT
TTAAAGAAGGAAGCTCCGGTGCCAGTCAGAGAGTCGCCGGCGACTCAGGCTTTGCC
GCGTATAGCCGATATCGGATTGGCAATGACGGTGTCCCGTTCGTGGTCTCGACCGG
ATATCACTTCCGTGAACTAGGGGTATATCTGACCTTCTACTTGACGAACGACGTGTC
GTTCCTAGCGCATATTCAATGGATGGTCATGTTCACGCCAGGCTTAATGTGGCTGTC
TTATTTCATCGCCTCTTTTAGACTGTTCGCTAGGACAAGGAGCATGTTTGAGTATTA
CCACACTACTGACCCCAGTTTTCTAGGACGCTACATGAGTGCTTTGAATCATACCA
AGAAGTGGAAATACCCACAGATTTCCAATGAGAAGCAGGAGATCCTTGGAACAGT
TAGTTGGAATTTGAGGGAAGCCACGACGAGACAGGTTGTGAACGTGGTTACGACA
AAAATCGCACTGAAGACACTAGCCTGCTTCGTGCTGGCCGCCGTTTACCGCATCAA
TTGGATTACCGGACCCGGACCAGGCGCCAAATTTGTTGCTGCTTGGACACTGAAAG
CTGCTGCTGGGCCCGGACCAGGCCAGTACATCAAGGCCAACTCTAAGTTTATCGGC
ATCACCGAATTGGGACCTGGACCCGGCTAGTGAttcgaaGggcccctataactctctacggctaacctga
atggactacgacatagtctagtccgccaaggccaccATGTTTGTCTTCCTGGTCTTGCTGCCGCTGGTGAGC
AGCCAGTGCGTGAATCTCACCACCCGCACCCAGCTTCCACCTGCCTACACTAACAGCTTC
ACCCGAGGGGTGTATTACCCTGACAAGGTATTCCGGTCCTCCGTCCTCCATAGCACGCA
GGACCTTTTTCTGCCCTTCTTCTCAAATGTGACATGGTTCCATGtgATTAGCGGCACGAAT
GGAACGAAGCGCTTTGATAACCCCGTGCTGCCTTTCAATGACGGCGTCTACTTCGCCTCC
AtTGAAAAGTCAAACATCATCCGGGGCTGGATCTTTGGCACCACTCTTGATTCAAAGACCC
AGTCACTGCTGATTGTGAACAATGCTACAAACGTGGTTATCAAGGTGTGTGAGTTTCAGTT
CTGTAACGATCCATTTTTGGacCACAAGAACAACAAGTCCTGGATGGAGTCTGAGTTCAGA
GTGTATAGCTCTGCTAACAACTGCACCTTCGAGTACGTGTCCCAGCCTTTCCTTATGGAC
CTGGAAGGCAAACAGGGCAATTTCAAAAACCTGAGAGAGTTCGTGTTTAAGAACATTGAC
GGATACTTCAAAATTTATTCTAAGCACACACCAATTaTtGTGCGGgagccagagGACCTACCCC
AAGGCTTTAGCGCCCTAGAGCCCCTGGTTGACCTGCCCATTGGGATCAATATAACAAGGT
TCCAAACTCTACTGGCTCTGCATAGAAGTTATCTGACCCCAGGAGACAGCTCTAGTGGTT
GGACCGCCGGCGCAGCAGCCTACTATGTCGGGTACTTACAGCCACGCACGTTCCTTCTG
AAGTACAATGAGAACGGGACAATCACTGACGCAGTAGACTGTGCACTGGACCCGCTAAG
CGAGACTAAGTGCACACTTAAATCCTTCACGGTGGAGAAAGGCATTTATCAGACCTCTAA
CTTCAGGGTGCAGCCAACAGAAAGCATTGTGCGATTCCCAAATATTACTAATCTTTGCCCT
TTCGacGAGGTCTTTAATGCAACTAGATTCGCATCAGTCTATGCGTGGAACCGCAAACGCA
TTTCCAATTGTGTCGCAGACTACTCAGTGCTGTACAACctgGCCcCTTTCttTACGTTCAAGT
GTTACGGAGTGTCACCCACTAAACTGAACGACCTGTGCTTTACAAATGTCTACGCTGACT
CCTTCGTGATTAGGGGAGACGAGGTGAGACAAATTGCCCCCGGACAGACTGGGAAcATT
GCCGACTACAATTATAAGCTTCCTGATGATTTCACTGGCTGTGTTATTGCCTGGAATAGTA
ACAAgCTGGATAGCAAGGTGaGcGGCAACTATAACTACTTATATCGACTGTTTAGGAAGAG
TAATCTGAAACCATTTGAGCGGGATATTTCCACAGAAATTTACCAGGCCGGGAaCAaACCA
TGTAATGGGGTGGccGGATTTAATTGTTACTTCCCACTCaAGAGCTATaGTTTCCgACCCAC
CtATGGAGTGGGTcACCAGCCCTATAGAGTCGTGGTGCTTAGTTTTGAGCTGCTTCACGC
CCCAGCAACCGTCTGCGGTCCCAAAAAGTCGACCAATCTCGTGAAAAACAAATGCGTAAA
CTTCAACTTTAACGGCTTAAaAGGAACCGGCGTGCTCACCGAAAGCAACAAGAAATTCCT
TCCATTTCAGCAATTCGGAAGGGACATCGCCGACACAACAGACGCGGTGAGGGACCCAC
AGACTCTGGAGATACTGGACATCACTCCTTGTTCGTTTGGGGGCGTCTCGGTCATCACAC
CCGGGACTAATACTAGTAATCAGGTAGCAGTTTTATATCAAGGCGTCAACTGTACCGAAG
TACCTGTGGCCATACACGCTGATCAGCTAACGCCAACATGGCGAGTCTATTCCACCGGCT
CTAACGTTTTTCAGACCAGGGCTGGGTGCCTGATAGGGGCAGAGtAtGTCAATAATTCCTA
TGAGTGTGATATCCCCATAGGTGCGGGGATCTGTGCCAGCTATCAAACCCAAACCAAaTC
ACacgGGaGcGCAaGcTCTGTGGCTTCTCAGAGCATAATTGCATATACAATGTCACTGGGC
GCTGAGAATAGCGTTGCATACTCTAATAACAGCATAGCCATTCCCACGAACTTTACTATCA
GTGTGACAACCGAAATATTGCCAGTTTCGATGACCAAAACTAGCGTGGATTGCACGATGT
ACATCTGTGGAGACTCTACCGAATGCAGCAATCTGCTATTACAATATGGCAGCTTCTGTAC
ACAGTTAAAgCGAGCCTTGACAGGCATCGCAGTGGAACAGGACAAAAATACTCAAGAGGT
GTTTGCACAGGTGAAGCAAATCTACAAAACGCCCCCCATTAAAtATTTTGGCGGGTTCAAT
TTTTCACAAATTCTCCCCGACCCGTCTAAGCCGAGTAAGCGGTCCTTCATCGAAGATCTG
CTCTTTAACAAAGTAACCCTCGCCGATGCCGGCTTTATTAAGCAGTATGGCGACTGCCTG
GGGGATATAGCCGCTCGTGACCTGATTTGCGCCCAGAAGTTCAAaGGTCTGACCGTGTT
GCCTCCTTTATTGACCGATGAAATGATTGCCCAGTACACTAGTGCCCTGCTGGCCGGCAC
TATCACGTCTGGGTGGACCTTCGGAGCTGGTGCCGCCTTGCAGATACCTTTTGCAATGCA
GATGGCCTATAGGTTTAATGGTATCGGAGTGACTCAGAACGTACTGTACGAGAACCAGAA
GCTCATCGCTAATCAATTTAACTCCGCTATCGGAAAAATCCAGGACAGCCTCTCTTCTACA
GCTAGCGCTCTGGGCAAACTGCAGGATGTCGTTAATCAtAATGCCCAGGCCCTGAACACC
TTGGTTAAACAACTATCTTCCAAaTTCGGGGCCATATCCAGTGTGTTGAATGATATTTTCTC
CCGCTTGGATccacctGAAGCTGAGGTGCAGATCGATCGCTTGATCACCGGCAGACTGCAG
TCCCTCCAGACATATGTAACTCAGCAGCTGATTAGAGCCGCCGAGATAAGGGCAAGTGC
GAATCTGGCTGCCACCAAGATGAGCGAATGTGTATTGGGCCAGAGCAAACGAGTTGATTT
TTGCGGTAAGGGGTATCATTTAATGTCTTTCCCTCAATCCGCACCTCATGGCGTAGTTTTC
CTGCATGTGACTTATGTCCCGGCTCAGGAGAAGAATTTTACCACAGCCCCCGCGATCTGC
CATGACGGAAAGGCCCACTTCCCCCGGGAAGGCGTGTTTGTATCCAATGGGACTCACTG
GTTTGTCACTCAGCGAAATTTTTATGAACCACAGATCATCACCACTGACAACACATTTGTT
AGTGGAAACTGCGATGTGGTCATCGGCATCGTGAATAACACTGTCTATGATCCACTGCAA
CCTGAACTGGATTCTTTTAAAGAGGAACTCGACAAGTATTTTAAAAACCACACTAGCCCTG
ACGTGGATCTCGGTGACATTTCTGGCATCAACGCTAGCGTAGTGAACATTCAGAAAGAGA
TAGATAGACTTAATGAGGTGGCCAAGAACCTCAACGAAAGTCTGATCGACCTCCAGGAAC
TGGGGAAATACGAGCAGTACATTAAATGGCCTTGGTACATATGGCTGGGGTTCATTGCTG
GGCTGATCGCAATAGTGATGGTGACCATAATGCTCTGTTGCATGACTAGCTGCTGCAGCT
GCCTGAAGGGCTGCTGTAGTTGTGGGTCATGTTGTAAGTTTGACGAAGATGATAGCGAG
CCTGTCCTTAAAGGAGTGAAGCTCCACTACACCTAGTAAttcgaacggccgtatcacgcccaaacatttac
agccgcggtgtcaaaaaccgcgtggacgtggttaacatccctgctgggaggatcagccgtaattattataattggcttggtgctggctacta
ttgtggccatgtacgtgctgaccaaccagaaacataattgaatacagcagcaattggcaagctgcttacatagaactcgcggcgattggca
tgccgccttaaaatttttattttattttttcttttcttttccgaatcggattttgtttttaatatttcaaaaaaaaaaaaaaaaaaaaaaaaaa
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa
a
SARS-COV-2 Spike Nucleotide Sequence (SEQ ID NO: 27980)
(BA5 “Omicron” variant; modified); Includes mutations 679-682 RRAR [SEQ ID NO: 125] to
GSAS [SEQ ID NO: 126], K983P and V984P; sequence optimized based on CT1 sequence
optimization with relevant variant mutations replaced with COOL codons;
ATGTTTGTCTTCCTGGTCTTGCTGCCGCTcGTGtctAGCCAGTGCGTGAATCTCAtCAC
CCGCACCCAGtccTACACTAACAGCTTCACCCGAGGGGTGTATTACCCTGACAAGGT
ATTCCGGTCCTCCGTCCTCCATAGCACGCAGGACCTTTTTCTGCCCTTCTTCTCAAA
TGTGACATGGTTCCATGCCATTAGCGGCACGAATGGAACGAAGCGCTTTGATAACC
CCGTGCTGCCTTTCAATGACGGCGTCTACTTCGCCTCCACTGAAAAGTCAAACATC
ATCCGGGGCTGGATCTTTGGCACCACTCTTGATTCAAAGACCCAGTCACTGCTGATT
GTGAACAATGCTACAAACGTGGTTATCAAGGTGTGTGAGTTTCAGTTCTGTAACGA
TCCATTTTTGGatGTGTACTACCACAAGAACAACAAGTCCTGGATGGAGTCTGAGTT
CAGAGTGTATAGCTCTGCTAACAACTGCACCTTCGAGTACGTGTCCCAGCCTTTCCT
TATGGACCTGGAAGGCAAACAGGGCAATTTCAAAAACCTGAGAGAGTTCGTGTTTA
AGAACATTGACGGATACTTCAAAATTTATTCTAAGCACACACCAATTAACTTAGgtC
GGGACCTACCCCAAGGCTTTAGCGCCCTAGAGCCCCTGGTTGACCTGCCCATTGGG
ATCAATATAACAAGGTTCCAAACTCTACTGGCTCTGCATAGAAGTTATCTGACCCC
AGGAGACAGCTCTAGTGGTTGGACCGCCGGCGCAGCAGCCTACTATGTCGGGTACT
TACAGCCACGCACGTTCCTTCTGAAGTACAATGAGAACGGGACAATCACTGACGCA
GTAGACTGTGCACTGGACCCGCTAAGCGAGACTAAGTGCACACTTAAATCCTTCAC
GGTGGAGAAAGGCATTTATCAGACCTCTAACTTCAGGGTGCAGCCAACAGAAAGC
ATTGTGCGATTCCCAAATATTACTAATCTTTGCCCTTTCGacGAGGTCTTTAATGCAA
CTAGATTCGCATCAGTCTATGCGTGGAACCGCAAACGCATTTCCAATTGTGTCGCA
GACTACTCAGTGCTGTACAACT(TGCCCCTTTCttTgCGTTCAAGTGTTACGGAGTGTC
ACCCACTAAACTGAACGACCTGTGCTTTACAAATGTCTACGCTGACTCCTTCGTGAT
TAGGGGAaACGAGGTGAGtCAAATTGCCCCCGGACAGACTGGGAAcATTGCCGACT
ACAATTATAAGCTTCCTGATGATTTCACTGGCTGTGTTATTGCCTGGAATAGTAACA
AgCTGGATAGCAAGGTGGGAGGCAACTATAACTACaggTATCGACTGTTTAGGAAGA
GTAATCTGAAACCATTTGAGCGGGATATTTCCACAGAAATTTACCAGGCCGGGAaC
AaACCATGTAATGGGGTGGccGGAgtcAATTGTTACTTCCCACTCCaGAGCTATGGTTT
CagACCCACCtATGGAGTGGGTcACCAGCCCTATAGAGTCGTGGTGCTTAGTTTTGAG
CTGCTTCACGCCCCAGCAACCGTCTGCGGTCCCAAAAAGTCGACCAATCTCGTGAA
AAACAAATGCGTAAACTTCAACTTTAACGGCTTAACAGGAACCGGCGTGCTCACCG
AAAGCAACAAGAAATTCCTTCCATTTCAGCAATTCGGAAGGGACATCGCCGACACA
ACAGACGCGGTGAGGGACCCACAGACTCTGGAGATACTGGACATCACTCCTTGTTC
GTTTGGGGGCGTCTCGGTCATCACACCCGGGACTAATACTAGTAATCAGGTAGCAG
TTTTATATCAAGGCGTCAACTGTACCGAAGTACCTGTGGCCATACACGCTGATCAG
CTAACGCCAACATGGCGAGTCTATTCCACCGGCTCTAACGTTTTTCAGACCAGGGC
TGGGTGCCTGATAGGGGCAGAGtAtGTCAATAATTCCTATGAGTGTGATATCCCCAT
AGGTGCGGGGATCTGTGCCAGCTATCAAACCCAAACCAAaTCACacgGGaGcGCAaGc
TCTGTGGCTTCTCAGAGCATAATTGCATATACAATGTCACTGGGCGCTGAGAATAG
CGTTGCATACTCTAATAACAGCATAGCCATTCCCACGAACTTTACTATCAGTGTGAC
AACCGAAATATTGCCAGTTTCGATGACCAAAACTAGCGTGGATTGCACGATGTACA
TCTGTGGAGACTCTACCGAATGCAGCAATCTGCTATTACAATATGGCAGCTTCTGT
ACACAGTTAAAgCGAGCCTTGACAGGCATCGCAGTGGAACAGGACAAAAATACTC
AAGAGGTGTTTGCACAGGTGAAGCAAATCTACAAAACGCCCCCCATTAAAtATTTT
GGCGGGTTCAATTTTTCACAAATTCTCCCCGACCCGTCTAAGCCGAGTAAGCGGTC
CTTCATCGAAGATCTGCTCTTTAACAAAGTAACCCTCGCCGATGCCGGCTTTATTAA
GCAGTATGGCGACTGCCTGGGGGATATAGCCGCTCGTGACCTGATTTGCGCCCAGA
AGTTCAATGGTCTGACCGTGTTGCCTCCTTTATTGACCGATGAAATGATTGCCCAGT
ACACTAGTGCCCTGCTGGCCGGCACTATCACGTCTGGGTGGACCTTCGGAGCTGGT
GCCGCCTTGCAGATACCTTTTGCAATGCAGATGGCCTATAGGTTTAATGGTATCGG
AGTGACTCAGAACGTACTGTACGAGAACCAGAAGCTCATCGCTAATCAATTTAACT
CCGCTATCGGAAAAATCCAGGACAGCCTCTCTTCTACAGCTAGCGCTCTGGGCAAA
CTGCAGGATGTCGTTAATCAtAATGCCCAGGCCCTGAACACCTTGGTTAAACAACTA
TCTTCCAAgTTCGGGGCCATATCCAGTGTGTTGAATGATATTCTCTCCCGCTTGGATc
cacctGAAGCTGAGGTGCAGATCGATCGCTTGATCACCGGCAGACTGCAGTCCCTCCA
GACATATGTAACTCAGCAGCTGATTAGAGCCGCCGAGATAAGGGCAAGTGCGAAT
CTGGCTGCCACCAAGATGAGCGAATGTGTATTGGGCCAGAGCAAACGAGTTGATTT
TTGCGGTAAGGGGTATCATTTAATGTCTTTCCCTCAATCCGCACCTCATGGCGTAGT
TTTCCTGCATGTGACTTATGTCCCGGCTCAGGAGAAGAATTTTACCACAGCCCCCGC
GATCTGCCATGACGGAAAGGCCCACTTCCCCCGGGAAGGCGTGTTTGTATCCAATG
GGACTCACTGGTTTGTCACTCAGCGAAATTTTTATGAACCACAGATCATCACCACT
GACAACACATTTGTTAGTGGAAACTGCGATGTGGTCATCGGCATCGTGAATAACAC
TGTCTATGATCCACTGCAACCTGAACTGGATTCTTTTAAAGAGGAACTCGACAAGT
ATTTTAAAAACCACACTAGCCCTGACGTGGATCTCGGTGACATTTCTGGCATCAAC
GCTAGCGTAGTGAACATTCAGAAAGAGATAGATAGACTTAATGAGGTGGCCAAGA
ACCTCAACGAAAGTCTGATCGACCTCCAGGAACTGGGGAAATACGAGCAGTACAT
TAAATGGCCTTGGTACATATGGCTGGGGTTCATTGCTGGGCTGATCGCAATAGTGA
TGGTGACCATAATGCTCTGTTGCATGACTAGCTGCTGCAGCTGCCTGAAGGGCTGC
TGTAGTTGTGGGTCATGTTGTAAGTTTGACGAAGATGATAGCGAGCCTGTCCTTAA
AGGAGTGAAGCTCCACTACACCTAGTAA
SAM-N-TCE11-Spike-beta samRNA; SEQ ID NO: 27981; “GRT-R912”
atgggggcgcatgagagaagcccagaccaattacctacccaaaatggagaaagttcacgttgacatcgaggaagacagcccattcctc
agagctttgcagcggagcttcccgcagtttgaggtagaagccaagcaggtcactgataatgaccatgctaatgccagagcgttttcgcatc
tggcttcaaaactgatcgaaacggaggtggacccatccgacacgatccttgacattggaagtgcgcccgcccgcagaatgtattctaagc
acaagtatcattgtatctgtccgatgagatgtgcggaagatccggacagattgtataagtatgcaactaagctgaagaaaaactgtaaggaa
ataactgataaggaattggacaagaaaatgaaggagctcgccgccgtcatgagcgaccctgacctggaaactgagactatgtgcctcca
cgacgacgagtcgtgtcgctacgaagggcaagtcgctgtttaccaggatgtatacgcggttgacggaccgacaagtctctatcaccaagc
caataagggagttagagtcgcctactggataggctttgacaccaccccttttatgtttaagaacttggctggagcatatccatcatactctacc
aactgggccgacgaaaccgtgttaacggctcgtaacataggcctatgcagctctgacgttatggagcggtcacgtagagggatgtccatt
cttagaaagaagtatttgaaaccatccaacaatgttctattctctgttggctcgaccatctaccacgagaagagggacttactgaggagctgg
cacctgccgtctgtatttcacttacgtggcaagcaaaattacacatgtcggtgtgagactatagttagttgcgacgggtacgtcgttaaaaga
atagctatcagtccaggcctgtatgggaagccttcaggctatgctgctacgatgcaccgcgagggattcttgtgctgcaaagtgacagaca
cattgaacggggagagggtctcttttcccgtgtgcacgtatgtgccagctacattgtgtgaccaaatgactggcatactggcaacagatgtc
agtgcggacgacgcgcaaaaactgctggttgggctcaaccagcgtatagtcgtcaacggtcgcacccagagaaacaccaataccatga
aaaattaccttttgcccgtagtggcccaggcatttgctaggtgggcaaaggaatataaggaagatcaagaagatgaaaggccactaggac
tacgagatagacagttagtcatggggtgttgttgggcttttagaaggcacaagataacatctatttataagcgcccggatacccaaaccatca
tcaaagtgaacagcgatttccactcattcgtgctgcccaggataggcagtaacacattggagatcgggctgagaacaagaatcaggaaaa
tgttagaggagcacaaggagccgtcacctctcattaccgccgaggacgtacaagaagctaagtgcgcagccgatgaggctaaggaggt
gcgtgaagccgaggagttgcgcgcagctctaccacctttggcagctgatgttgaggagcccactctggaagccgatgtcgacttgatgtta
caagaggctggggccggctcagtggagacacctcgtggcttgataaaggttaccagctacgctggcgaggacaagatcggctcttacgc
tgtgctttctccgcaggctgtactcaagagtgaaaaattatcttgcatccaccctctcgctgaacaagtcatagtgataacacactctggccg
aaaagggcgttatgccgtggaaccataccatggtaaagtagtggtgccagagggacatgcaatacccgtccaggactttcaagctctgag
tgaaagtgccaccattgtgtacaacgaacgtgagttcgtaaacaggtacctgcaccatattgccacacatggaggagcgctgaacactgat
gaagaatattacaaaactgtcaagcccagcgagcacgacggcgaatacctgtacgacatcgacaggaaacagtgcgtcaagaaagaac
tagtcactgggctagggctcacaggcgagctggtggatcctcccttccatgaattcgcctacgagagtctgagaacacgaccagccgctc
cttaccaagtaccaaccataggggtgtatggcgtgccaggatcaggcaagtctggcatcattaaaagcgcagtcaccaaaaaagatctag
tggtgagcgccaagaaagaaaactgtgcagaaattataagggacgtcaagaaaatgaaagggctggacgtcaatgccagaactgtgga
ctcagtgctcttgaatggatgcaaacaccccgtagagaccctgtatattgacgaagcttttgcttgtcatgcaggtactctcagagcgctcat
agccattataagacctaaaaaggcagtgctctgcggggatcccaaacagtgcggtttttttaacatgatgtgcctgaaagtgcattttaacca
cgagatttgcacacaagtcttccacaaaagcatctctcgccgttgcactaaatctgtgacttcggtcgtctcaaccttgttttacgacaaaaaa
atgagaacgacgaatccgaaagagactaagattgtgattgacactaccggcagtaccaaacctaagcaggacgatctcattctcacttgttt
cagagggtgggtgaagcagttgcaaatagattacaaaggcaacgaaataatgacggcagctgcctctcaagggctgacccgtaaaggt
gtgtatgccgttcggtacaaggtgaatgaaaatcctctgtacgcacccacctcagaacatgtgaacgtcctactgacccgcacggaggac
cgcatcgtgtggaaaacactagccggcgacccatggataaaaacactgactgccaagtaccctgggaatttcactgccacgatagagga
gtggcaagcagagcatgatgccatcatgaggcacatcttggagagaccggaccctaccgacgtcttccagaataaggcaaacgtgtgtt
gggccaaggctttagtgccggtgctgaagaccgctggcatagacatgaccactgaacaatggaacactgtggattattttgaaacggaca
aagctcactcagcagagatagtattgaaccaactatgcgtgaggttctttggactcgatctggactccggtctattttctgcacccactgttcc
gttatccattaggaataatcactgggataactccccgtcgcctaacatgtacgggctgaataaagaagtggtccgtcagctctctcgcaggt
acccacaactgcctcgggcagttgccactggaagagtctatgacatgaacactggtacactgcgcaattatgatccgcgcataaacctagt
acctgtaaacagaagactgcctcatgctttagtcctccaccataatgaacacccacagagtgacttttcttcattcgtcagcaaattgaaggg
cagaactgtcctggtggtcggggaaaagttgtccgtcccaggcaaaatggttgactggttgtcagaccggcctgaggctaccttcagagct
cggctggatttaggcatcccaggtgatgtgcccaaatatgacataatatttgttaatgtgaggaccccatataaataccatcactatcagcagt
gtgaagaccatgccattaagcttagcatgttgaccaagaaagcttgtctgcatctgaatcccggcggaacctgtgtcagcataggttatggtt
acgctgacagggccagcgaaagcatcattggtgctatagcgcggcagttcaagttttcccgggtatgcaaaccgaaatcctcacttgaag
agacggaagttctgtttgtattcattgggtacgatcgcaaggcccgtacgcacaatccttacaagctttcatcaaccttgaccaacatttatac
aggttccagactccacgaagccggatgtgcaccctcatatcatgtggtgcgaggggatattgccacggccaccgaaggagtgattataaa
tgctgctaacagcaaaggacaacctggcggaggggtgtgcggagcgctgtataagaaattcccggaaagcttcgatttacagccgatcg
aagtaggaaaagcgcgactggtcaaaggtgcagctaaacatatcattcatgccgtaggaccaaacttcaacaaagtttcggaggttgaag
gtgacaaacagttggcagaggcttatgagtccatcgctaagattgtcaacgataacaattacaagtcagtagcgattccactgttgtccacc
ggcatcttttccgggaacaaagatcgactaacccaatcattgaaccatttgctgacagctttagacaccactgatgcagatgtagccatatac
tgcagggacaagaaatgggaaatgactctcaaggaagcagtggctaggagagaagcagtggaggagatatgcatatccgacgactctt
cagtgacagaacctgatgcagagctggtgagggtgcatccgaagagttctttggctggaaggaagggctacagcacaagcgatggcaa
aactttctcatatttggaagggaccaagtttcaccaggcggccaaggatatagcagaaattaatgccatgtggcccgttgcaacggaggcc
aatgagcaggtatgcatgtatatcctcggagaaagcatgagcagtattaggtcgaaatgccccgtcgaagagtcggaagcctccacacca
cctagcacgctgccttgcttgtgcatccatgccatgactccagaaagagtacagcgcctaaaagcctcacgtccagaacaaattactgtgt
gctcatcctttccattgccgaagtatagaatcactggtgtgcagaagatccaatgctcccagcctatattgttctcaccgaaagtgcctgcgta
tattcatccaaggaagtatctcgtggaaacaccaccggtagacgagactccggagccatcggcagagaaccaatccacagaggggaca
cctgaacaaccaccacttataaccgaggatgagaccaggactagaacgcctgagccgatcatcatcgaagaggaagaagaggatagca
taagtttgctgtcagatggcccgacccaccaggtgctgcaagtcgaggcagacattcacgggccgccctctgtatctagctcatcctggtc
cattcctcatgcatccgactttgatgtggacagtttatccatacttgacaccctggagggagctagcgtgaccagcggggcaacgtcagcc
gagactaactcttacttcgcaaagagtatggagtttctggcgcgaccggtgcctgcgcctcgaacagtattcaggaaccctccacatcccg
ctccgcgcacaagaacaccgtcacttgcacccagcagggcctgctcgagaaccagcctagtttccaccccgccaggcgtgaatagggt
gatcactagagaggagctcgaggcgcttaccccgtcacgcactcctagcaggtcggtctcgagaaccagcctggtctccaacccgccag
gcgtaaatagggtgattacaagagaggagtttgaggcgttcgtagcacaacaacaatgacggtttgatgcgggtgcatacatcttttcctcc
gacaccggtcaagggcatttacaacaaaaatcagtaaggcaaacggtgctatccgaagtggtgttggagaggaccgaattggagatttcg
tatgccccgcgcctcgaccaagaaaaagaagaattactacgcaagaaattacagttaaatcccacacctgctaacagaagcagataccag
tccaggaaggtggagaacatgaaagccataacagctagacgtattctgcaaggcctagggcattatttgaaggcagaaggaaaagtgga
gtgctaccgaaccctgcatcctgttcctttgtattcatctagtgtgaaccgtgccttttcaagccccaaggtcgcagtggaagcctgtaacgc
catgttgaaagagaactttccgactgtggcttcttactgtattattccagagtacgatgcctatttggacatggttgacggagcttcatgctg
cttagacactgccagtttttgccctgcaaagctgcgcagctttccaaagaaacactcctatttggaacccacaatacgatcggcagtgccttca
gcgatccagaacacgctccagaacgtcctggcagctgccacaaaaagaaattgcaatgtcacgcaaatgagagaattgcccgtattggatt
cggcggcctttaatgtggaatgcttcaagaaatatgcgtgtaataatgaatattgggaaacgtttaaagaaaaccccatcaggcttactgaag
aaaacgtggtaaattacattaccaaattaaaaggaccaaaagctgctgctctttttgcgaagacacataatttgaatatgttgcaggacatacc
aatggacaggtttgtaatggacttaaagagagacgtgaaagtgactccaggaacaaaacatactgaagaacggcccaaggtacaggtga
tccaggctgccgatccgctagcaacagcgtatctgtgcggaatccaccgagagctggttaggagattaaatgcggtcctgcttccgaacat
tcatacactgtttgatatgtcggctgaagactttgacgctattatagccgagcacttccagcctggggattgtgttctggaaactgacatcgcg
tcgtttgataaaagtgaggacgacgccatggctctgaccgcgttaatgattctggaagacttaggtgtggacgcagagctgttgacgctgat
tgaggcggctttcggcgaaatttcatcaatacatttgcccactaaaactaaatttaaattcggagccatgatgaaatctggaatgttcctcaca
ctgtttgtgaacacagtcattaacattgtaatcgcaagcagagtgttgagagaacggctaaccggatcaccatgtgcagcattcattggaga
tgacaatatcgtgaaaggagtcaaatcggacaaattaatggcagacaggtgcgccacctggttgaatatggaagtcaagattatagatgct
gtggtgggcgagaaagcgccttatttctgtggagggtttattttgtgtgactccgtgaccggcacagcgtgccgtgtggcagaccccctaa
aaaggctgtttaagcttggcaaacctctggcagcagacgatgaacatgatgatgacaggagaagggcattgcatgaagagtcaacacgc
tggaaccgagtgggtattctttcagagctgtgcaaggcagtagaatcaaggtatgaaaccgtaggaacttccatcatagttatggccatgac
tactctagctagcagtgttaaatcattcagctacctgagaggggcccctataactctctacggctaacctgaatggactacgactctagaata
gtctttaattaaGCCACCATGTCAGATAATGGGCCTCAGAACCAGCGGAATGCCCCCAGGA
TCACGTTCGGTGGGCCTTCAGATTCAACTGGAAGCAATCAGAACGGGGAAAGATCC
GGTGCAAGGTCCAAGCAACGCCGACCCCAGGGATTGCCTAATAATACAGCAAGTT
GGTTTACTGCACTGACCCAACACGGGAAAGAGGACCTTAAATTTCCGCGGGGGCA
AGGAGTACCTATAAACACTAATAGCAGCCCCGATGACCAAATTGGGTACTACCGCA
GAGCAACCCGGCGAATCAGAGGGGGTGACGGTAAGATGAAGGACCTCTCCCCCAG
GTGGTACTTCTACTATTTGGGAACGGGGCCAGAAGCCGGACTGCCCTACGGCGCAA
AcAAAGACGGTATAATATGGGTCGCCACTGAGGGCGCTCTGAATACGCCAAAAGAT
CACATTGGTACTAGAAACCCGGCGAACAATGCGGCAATTGTGCTTCAGCTCCCTCA
AGGAACAACGTTGCCAAAAGGATTTTACGCAGAGGGATCACGGGGGGGTTCCCAG
GCTAGTTCTCGCAGTTCCTCAAGGAGTCGAAATTCCAGTAGGAAcTCAACCCCAGG
CTCAAGCCGCGGGACTTCACCTGCAAGAATGGCCGGTAACGGGGGAGACGCTGCT
TTGGCTCTTCTTCTCCTTGATCGCCTTAATCAGCTTGAGTCTAAGATGTCTGGTAAA
GGCCAACAGCAACAAGGACAGACGGTAACGAAAAAATCCGCCGCCGAGGCCTCTA
AAAAACCGAGACAAAAACGGACTGCTACGAAAGCGTATAACGTCACTCAGGCTTT
TGGTAGGCGGGGCCCGGAACAAACCCAGGGTAATTTTGGCGATCAGGAACTGATT
AGACAAGGCACCGACTACAAGCACTGGCCTCAGATCGCGCAGTTTGCACCCTCCGC
TTCAGCGTTCTTCGGGATGTCACGCATCGGGATGGAGGTTACACCGAGTGGAACAT
GGCTGACATATACTGGTGCAATcAAGCTGGACGACAAGGATCCCAACTTCAAAGAT
CAAGTTATACTCCTCAACAAACATATCGATGCGTATAAGACATTCCCCCCGACGGA
ACCCAAAAAAGATAAAAAAAAGAAGGCCGACGAGACCCAAGCCCTCCCACAACG
ACAAAAGAAACAACAAACAGTAACACTGCTCCCAGCCGCAGACTTGGATGATTTCT
CCAAACAGTTGCAGCAATCTATGAGTTCAGCCGATAGTACTCAGGCCGGCGGTTCT
GGAGGTGTCCGTGCTGAAGGACGCGGGTCCCTTCTCACCTGCGGCGACGTTGAGGA
AAACCCTGGCCCTATGGCTGGCAACGTAGCTTTCCAGACCGTGAAGCCGGGAAATT
TCAACAAGGACTTCTACGACTTCGCCGTCAGCAAGGGATTCTTTAAAGAAGGAAGC
TCCGGTGCCAGTCAGAGAGTCGCCGGCGACTCAGGCTTTGCCGCGTATAGCCGATA
TCGGATTGGCAATGACGGTGTCCCGTTCGTGGTCTCGACCGGATATCACTTCCGTG
AACTAGGGGTATATCTGACCTTCTACTTGACGAACGACGTGTCGTTCCTAGCGCAT
ATTCAATGGATGGTCATGTTCACGCCAGGCTTAATGTGGCTGTCTTATTTCATCGCC
TCTTTTAGACTGTTCGCTAGGACAAGGAGCATGTTTGAGTATTACCACACTACTGAC
CCCAGTTTTCTAGGACGCTACATGAGTGCTTTGAATCATACCAAGAAGTGGAAATA
CCCACAGATTTCCAATGAGAAGCAGGAGATCCTTGGAACAGTTAGTTGGAATTTGA
GGGAAGCCACGACGAGACAGGTTGTGAACGTGGTTACGACAAAAATCGCACTGAA
GACACTAGCCTGCTTCGTGCTGGCCGCCGTTTACCGCATCAATTGGATTACCGGAC
CCGGACCAGGCGCCAAATTTGTTGCTGCTTGGACACTGAAAGCTGCTGCTGGGCCC
GGACCAGGCCAGTACATCAAGGCCAACTCTAAGTTTATCGGCATCACCGAATTGGG
ACCTGGACCCGGCTAGTGAttcgaagggcccctataactctctacggctaacctgaatggactacgacatagtctagtcc
gccaagGCCACCATGTTTGTCTTCCTGGTCTTGCTGCCGCTGGTGAGCAGCCAGTGCGT
GAATTTCACCACCCGCACCCAGCTTCCACCTGCCTACACTAACAGCTTCACCCGAGG
GGTGTATTACCCTGACAAGGTATTCCGGTCCTCCGTCCTCCATAGCACGCAGGACC
TTTTTCTGCCCTTCTTCTCAAATGTGACATGGTTCCATGCCATTCACGTGAGCGGCA
CGAATGGAACGAAGCGCTTTGcTAACCCCGTGCTGCCTTTCAATGACGGCGTCTACT
TCGCCTCCACTGAAAAGTCAAACATCATCCGGGGCTGGATCTTTGGCACCACTCTT
GATTCAAAGACCCAGTCACTGCTGATTGTGAACAATGCTACAAACGTGGTTATCAA
GGTGTGTGAGTTTCAGTTCTGTAACGATCCATTTTTGGGAGTGTACTACCACAAGA
ACAACAAGTCCTGGATGGAGTCTGAGTTCAGAGTGTATAGCTCTGCTAACAACTGC
ACCTTCGAGTACGTGTCCCAGCCTTTCCTTATGGACCTGGAAGGCAAACAGGGCAA
TTTCAAAAACCTGAGAGAGTTCGTGTTTAAGAACATTGACGGATACTTCAAAATTT
ATTCTAAGCACACACCAATTAACTTAGTGCGGGgCCTACCCCAAGGCTTTAGCGCC
CTAGAGCCCCTGGTTGACCTGCCCATTGGGATCAATATAACAAGGTTCCAAACTCT
ACATAtAAGTTATCTGACCCCAGGAGACAGCTCTAGTGGTTGGACCGCCGGCGCAG
CAGCCTACTATGTCGGGTACTTACAGCCACGCACGTTCCTTCTGAAGTACAATGAG
AACGGGACAATCACTGACGCAGTAGACTGTGCACTGGACCCGCTAAGCGAGACTA
AGTGCACACTTAAATCCTTCACGGTGGAGAAAGGCATTTATCAGACCTCTAACTTC
AGGGTGCAGCCAACAGAAAGCATTGTGCGATTCCCAAATATTACTAATCTTTGCCC
TTTCGGGGAGGTCTTTAATGCAACTAGATTCGCATCAGTCTATGCGTGGAACCGCA
AACGCATTTCCAATTGTGTCGCAGACTACTCAGTGCTGTACAACTCTGCCTCTTTCA
GTACGTTCAAGTGTTACGGAGTGTCACCCACTAAACTGAACGACCTGTGCTTTACA
AATGTCTACGCTGACTCCTTCGTGATTAGGGGAGACGAGGTGAGACAAATTGCCCC
CGGACAGACTGGGAAtATTGCCGACTACAATTATAAGCTTCCTGATGATTTCACTGG
CTGTGTTATTGCCTGGAATAGTAACAATCTGGATAGCAAGGTGGGAGGCAACTATA
ACTACTTATATCGACTGTTTAGGAAGAGTAATCTGAAACCATTTGAGCGGGATATT
TCCACAGAAATTTACCAGGCCGGGAGCACACCATGTAATGGGGTGaAGGGATTTAA
TTGTTACTTCCCACTCCAGAGCTATGGTTTCCAACCCACCtATGGAGTGGGTTACCA
GCCCTATAGAGTCGTGGTGCTTAGTTTTGAGCTGCTTCACGCCCCAGCAACCGTCTG
CGGTCCCAAAAAGTCGACCAATCTCGTGAAAAACAAATGCGTAAACTTCAACTTTA
ACGGCTTAACAGGAACCGGCGTGCTCACCGAAAGCAACAAGAAATTCCTTCCATTT
CAGCAATTCGGAAGGGACATCGCCGACACAACAGACGCGGTGAGGGACCCACAGA
CTCTGGAGATACTGGACATCACTCCTTGTTCGTTTGGGGGCGTCTCGGTCATCACAC
CCGGGACTAATACTAGTAATCAGGTAGCAGTTTTATATCAAGGCGTCAACTGTACC
GAAGTACCTGTGGCCATACACGCTGATCAGCTAACGCCAACATGGCGAGTCTATTC
CACCGGCTCTAACGTTTTTCAGACCAGGGCTGGGTGCCTGATAGGGGCAGAGCACG
TCAATAATTCCTATGAGTGTGATATCCCCATAGGTGCGGGGATCTGTGCCAGCTAT
CAAACCCAAACCAATTCACCAgGGaGcGCAaGcTCTGTGGCTTCTCAGAGCATAATTG
CATATACAATGTCACTGGGCGTGAGAATAGCGTTGCATACTCTAATAACAGCATA
GCCATTCCCACGAACTTTACTATCAGTGTGACAACCGAAATATTGCCAGTTTCGAT
GACCAAAACTAGCGTGGATTGCACGATGTACATCTGTGGAGACTCTACCGAATGCA
GCAATCTGCTATTACAATATGGCAGCTTCTGTACACAGTTAAATCGAGCCTTGACA
GGCATCGCAGTGGAACAGGACAAAAATACTCAAGAGGTGTTTGCACAGGTGAAGC
AAATCTACAAAACGCCCCCCATTAAAGATTTTGGCGGGTTCAATTTTTCACAAATTC
TCCCCGACCCGTCTAAGCCGAGTAAGCGGTCCTTCATCGAAGATCTGCTCTTTAAC
AAAGTAACCCTCGCCGATGCCGGCTTTATTAAGCAGTATGGCGACTGCCTGGGGGA
TATAGCCGCTCGTGACCTGATTTGCGCCCAGAAGTTCAATGGTCTGACCGTGTTGCC
TCCTTTATTGACCGATGAAATGATTGCCCAGTACACTAGTGCCCTGCTGGCCGGCA
CTATCACGTCTGGGTGGACCTTCGGAGCTGGTGCCGCCTTGCAGATACCTTTTGCAA
TGCAGATGGCCTATAGGTTTAATGGTATCGGAGTGACTCAGAACGTACTGTACGAG
AACCAGAAGCTCATCGCTAATCAATTTAACTCCGCTATCGGAAAAATCCAGGACAG
CCTCTCTTCTACAGCTAGCGCTCTGGGCAAACTGCAGGATGTCGTTAATCAGAATG
CCCAGGCCCTGAACACCTTGGTTAAACAACTATCTTCCAACTTCGGGGCCATATCC
AGTGTGTTGAATGATATTCTCTCCCGCTTGGATccacctGAAGCTGAGGTGCAGATCGA
TCGCTTGATCACCGGCAGACTGCAGTCCCTCCAGACATATGTAACTCAGCAGCTGA
TTAGAGCCGCCGAGATAAGGGCAAGTGCGAATCTGGCTGCCACCAAGATGAGCGA
ATGTGTATTGGGCCAGAGCAAACGAGTTGATTTTTGCGGTAAGGGGTATCATTTAA
TGTCTTTCCCTCAATCCGCACCTCATGGCGTAGTTTTCCTGCATGTGACTTATGTCCC
GGCTCAGGAGAAGAATTTTACCACAGCCCCCGCGATCTGCCATGACGGAAAGGCC
CACTTCCCCCGGGAAGGCGTGTTTGTATCCAATGGGACTCACTGGTTTGTCACTCAG
CGAAATTTTTATGAACCACAGATCATCACCACTGACAACACATTTGTTAGTGGAAA
CTGCGATGTGGTCATCGGCATCGTGAATAACACTGTCTATGATCCACTGCAACCTG
AACTGGATTCTTTTAAAGAGGAACTCGACAAGTATTTTAAAAACCACACTAGCCCT
GACGTGGATCTCGGTGACATTTCTGGCATCAACGCTAGCGTAGTGAACATTCAGAA
AGAGATAGATAGACTTAATGAGGTGGCCAAGAACCTCAACGAAAGTCTGATCGAC
CTCCAGGAACTGGGGAAATACGAGCAGTACATTAAATGGCCTTGGTACATATGGCT
GGGGTTCATTGCTGGGCTGATCGCAATAGTGATGGTGACCATAATGCTCTGTTGCA
TGACTAGCTGCTGCAGCTGCCTGAAGGGCTGCTGTAGTTGTGGGTCATGTTGTAAG
TTTGACGAAGATGATAGCGAGCCTGTCCTTAAAGGAGTGAAGCTCCACTACACCTA
GTAAttcgaacggccgtatcacgcccaaacatttacagccgcggtgtcaaaaaccgcgtggacgtggttaacatccctgctgggagg
atcagccgtaattattataattggcttggtgctggctactattgtggccatgtacgtgctgaccaaccagaaacataattgaatacagcagcaa
ttggcaagctgcttacatagaactcgcggcgattggcatgccgccttaaaatttttattttattttttcttttttttccgaatcggattttgtt
tttaatatttcaaaaaaaaaaaaaaaaaaaaaaaaaaaaaaaaaaaaaaaaaaaaaaaaaaaaaaaaaaaaaaaaaaaaaaaaaaaaaaaa
SAM-TCE9-Spike-beta samRNA; SEQ ID NO: 27982 (TCE9 in bold; Spike in italic); “GRT-
R914”
atgggcggcgcatgagagaagcccagaccaattacctacccaaaatggagaaagttcacgttgacatcgaggaagacagcccattcctc
agagctttgcagcggagcttcccgcagtttgaggtagaagccaagcaggtcactgataatgaccatgctaatgccagagcgttttcgcatc
tggcttcaaaactgatcgaaacggaggtggacccatccgacacgatccttgacattggaagtgcgcccgcccgcagaatgtattctaagc
acaagtatcattgtatctgtccgatgagatgtgcggaagatccggacagattgtataagtatgcaactaagctgaagaaaaactgtaaggaa
ataactgataaggaattggacaagaaaatgaaggagctcgccgccgtcatgagcgaccctgacctggaaactgagactatgtgcctcca
cgacgacgagtcgtgtcgctacgaagggcaagtcgctgtttaccaggatgtatacgcggttgacggaccgacaagtctctatcaccaagc
caataagggagttagagtcgcctactggataggctttgacaccaccccttttatgtttaagaacttggctggagcatatccatcatactctacc
aactgggccgacgaaaccgtgttaacggctcgtaacataggcctatgcagctctgacgttatggagcggtcacgtagagggatgtccatt
cttagaaagaagtatttgaaaccatccaacaatgttctattctctgttggctcgaccatctaccacgagaagagggacttactgaggagctgg
cacctgccgtctgtatttcacttacgtggcaagcaaaattacacatgtcggtgtgagactatagttagttgcgacgggtacgtcgttaaaaga
atagctatcagtccaggcctgtatgggaagccttcaggctatgctgctacgatgcaccgcgagggattcttgtgctgcaaagtgacagaca
cattgaacggggagagggtctcttttcccgtgtgcacgtatgtgccagctacattgtgtgaccaaatgactggcatactggcaacagatgtc
agtgcggacgacgcgcaaaaactgctggttgggctcaaccagcgtatagtcgtcaacggtcgcacccagagaaacaccaataccatga
aaaattaccttttgcccgtagtggcccaggcatttgctaggtgggcaaaggaatataaggaagatcaagaagatgaaaggccactaggac
tacgagatagacagttagtcatggggtgttgttgggcttttagaaggcacaagataacatctatttataagcgcccggatacccaaaccatca
tcaaagtgaacagcgatttccactcattcgtgctgcccaggataggcagtaacacattggagatcgggctgagaacaagaatcaggaaaa
tgttagaggagcacaaggagccgtcacctctcattaccgccgaggacgtacaagaagctaagtgcgcagccgatgaggctaaggaggt
gcgtgaagccgaggagttgcgcgcagctctaccacctttggcagctgatgttgaggagcccactctggaagccgatgtcgacttgatgtta
caagaggctggggccggctcagtggagacacctcgtggcttgataaaggttaccagctacgctggcgaggacaagatcggctcttacgc
tgtgctttctccgcaggctgtactcaagagtgaaaaattatcttgcatccaccctctcgctgaacaagtcatagtgataacacactctggccg
aaaagggcgttatgccgtggaaccataccatggtaaagtagtggtgccagagggacatgcaatacccgtccaggactttcaagctctgag
tgaaagtgccaccattgtgtacaacgaacgtgagttcgtaaacaggtacctgcaccatattgccacacatggaggagcgctgaacactgat
gaagaatattacaaaactgtcaagcccagcgagcacgacggcgaatacctgtacgacatcgacaggaaacagtgcgtcaagaaagaac
tagtcactgggctagggctcacaggcgagctggtggatcctcccttccatgaattcgcctacgagagtctgagaacacgaccagccgctc
cttaccaagtaccaaccataggggtgtatggcgtgccaggatcaggcaagtctggcatcattaaaagcgcagtcaccaaaaaagatctag
tggtgagcgccaagaaagaaaactgtgcagaaattataagggacgtcaagaaaatgaaagggctggacgtcaatgccagaactgtgga
ctcagtgctcttgaatggatgcaaacaccccgtagagaccctgtatattgacgaagcttttgcttgtcatgcaggtactctcagagcgctcat
agccattataagacctaaaaaggcagtgctctgcggggatcccaaacagtgcggtttttttaacatgatgtgcctgaaagtgcattttaacca
cgagatttgcacacaagtcttccacaaaagcatctctcgccgttgcactaaatctgtgacttcggtcgtctcaaccttgttttacgacaaaaaa
atgagaacgacgaatccgaaagagactaagattgtgattgacactaccggcagtaccaaacctaagcaggacgatctcattctcacttgttt
cagaggggggtgaagcagttgcaaatagattacaaaggcaacgaaataatgacggcagctgcctctcaagggctgacccgtaaaggt
gtgtatgccgttcggtacaaggtgaatgaaaatcctctgtacgcacccacctcagaacatgtgaacgtcctactgacccgcacggaggac
cgcatcgtgtggaaaacactagccggcgacccatggataaaaacactgactgccaagtaccctgggaatttcactgccacgatagagga
gtggcaagcagagcatgatgccatcatgaggcacatcttggagagaccggaccctaccgacgtcttccagaataaggcaaacgtgtgtt
gggccaaggctttagtgccggtgctgaagaccgctggcatagacatgaccactgaacaatggaacactgtggattattttgaaacggaca
aagctcactcagcagagatagtattgaaccaactatgcgtgaggttctttggactcgatctggactccggtctattttctgcacccactgttcc
gttatccattaggaataatcactgggataactccccgtcgcctaacatgtacgggctgaataaagaagtggtccgtcagctctctcgcaggt
acccacaactgcctcgggcagttgccactggaagagtctatgacatgaacactggtacactgcgcaattatgatccgcgcataaacctagt
acctgtaaacagaagactgcctcatgctttagtcctccaccataatgaacacccacagagtgacttttcttcattcgtcagcaaattgaaggg
cagaactgtcctggtggtcggggaaaagttgtccgtcccaggcaaaatggttgactggttgtcagaccggcctgaggctaccttcagagct
cggctggatttaggcatcccaggtgatgtgcccaaatatgacataatatttgttaatgtgaggaccccatataaataccatcactatcagcagt
gtgaagaccatgccattaagcttagcatgttgaccaagaaagcttgtctgcatctgaatcccggcggaacctgtgtcagcataggttatggtt
acgctgacagggccagcgaaagcatcattggtgctatagcgcggcagttcaagttttcccgggtatgcaaaccgaaatcctcacttgaag
agacggaagttctgtttgtattcattgggtacgatcgcaaggcccgtacgcacaatccttacaagctttcatcaaccttgaccaacatttatac
aggttccagactccacgaagccggatgtgcaccctcatatcatgtggtgcgaggggatattgccacggccaccgaaggagtgattataaa
tgctgctaacagcaaaggacaacctggcggaggggtgtgcggagcgctgtataagaaattcccggaaagcttcgatttacagccgatcg
aagtaggaaaagcgcgactggtcaaaggtgcagctaaacatatcattcatgccgtaggaccaaacttcaacaaagtttcggaggttgaag
gtgacaaacagttggcagaggcttatgagtccatcgctaagattgtcaacgataacaattacaagtcagtagcgattccactgttgtccacc
ggcatcttttccgggaacaaagatcgactaacccaatcattgaaccatttgctgacagctttagacaccactgatgcagatgtagccatatac
tgcagggacaagaaatgggaaatgactctcaaggaagcagtggctaggagagaagcagtggaggagatatgcatatccgacgactctt
cagtgacagaacctgatgcagagctggtgagggtgcatccgaagagttctttggctggaaggaagggctacagcacaagcgatggcaa
aactttctcatatttggaagggaccaagtttcaccaggcggccaaggatatagcagaaattaatgccatgtggcccgttgcaacggaggcc
aatgagcaggtatgcatgtatatcctcggagaaagcatgagcagtattaggtcgaaatgccccgtcgaagagtcggaagcctccacacca
cctagcacgctgccttgcttgtgcatccatgccatgactccagaaagagtacagcgcctaaaagcctcacgtccagaacaaattactgtgt
gctcatcctttccattgccgaagtatagaatcactggtgtgcagaagatccaatgctcccagcctatattgttctcaccgaaagtgcctgcgta
tattcatccaaggaagtatctcgtggaaacaccaccggtagacgagactccggagccatcggcagagaaccaatccacagaggggaca
cctgaacaaccaccacttataaccgaggatgagaccaggactagaacgcctgagccgatcatcatcgaagaggaagaagaggatagca
taagtttgctgtcagatggcccgacccaccaggtgctgcaagtcgaggcagacattcacgggccgccctctgtatctagctcatcctggtc
cattcctcatgcatccgactttgatgtggacagtttatccatacttgacaccctggagggagctagcgtgaccagcggggcaacgtcagcc
gagactaactcttacttcgcaaagagtatggagtttctggcgcgaccggtgcctgcgcctcgaacagtattcaggaaccctccacatcccg
ctccgcgcacaagaacaccgtcacttgcacccagcagggcctgctcgagaaccagcctagtttccaccccgccaggcgtgaatagggt
gatcactagagaggagctcgaggcgcttaccccgtcacgcactcctagcaggtcggtctcgagaaccagcctggtctccaacccgccag
gcgtaaatagggtgattacaagagaggagtttgaggcgttcgtagcacaacaacaatgacggtttgatgcgggtgcatacatcttttcctcc
gacaccggtcaagggcatttacaacaaaaatcagtaaggcaaacggtgctatccgaagtggtgttggagaggaccgaattggagatttcg
tatgccccgcgcctcgaccaagaaaaagaagaattactacgcaagaaattacagttaaatcccacacctgctaacagaagcagataccag
tccaggaaggtggagaacatgaaagccataacagctagacgtattctgcaaggcctagggcattatttgaaggcagaaggaaaagtgga
gtgctaccgaaccctgcatcctgttcctttgtattcatctagtgtgaaccgtgccttttcaagccccaaggtcgcagtggaagcctgtaacgc
catgttgaaagagaactttccgactgtggcttcttactgtattattccagagtacgatgcctatttggacatggttgacggagcttcatgctgc
ttagacactgccagtttttgccctgcaaagctgcgcagctttccaaagaaacactcctatttggaacccacaatacgatcggcagtgccttcag
cgatccagaacacgctccagaacgtcctggcagctgccacaaaaagaaattgcaatgtcacgcaaatgagagaattgcccgtattggatt
cggcggcctttaatgtggaatgcttcaagaaatatgcgtgtaataatgaatattgggaaacgtttaaagaaaaccccatcaggcttactgaag
aaaacgtggtaaattacattaccaaattaaaaggaccaaaagctgctgctctttttgcgaagacacataatttgaatatgttgcaggacatacc
aatggacaggtttgtaatggacttaaagagagacgtgaaagtgactccaggaacaaaacatactgaagaacggcccaaggtacaggtga
tccaggctgccgatccgctagcaacagcgtatctgtgcggaatccaccgagagctggttaggagattaaatgcggtcctgcttccgaacat
tcatacactgtttgatatgtcggctgaagactttgacgctattatagccgagcacttccagcctggggattgtgttctggaaactgacatcgcg
tcgtttgataaaagtgaggacgacgccatggctctgaccgcgttaatgattctggaagacttaggtgtggacgcagagctgttgacgctgat
tgaggcggctttcggcgaaatttcatcaatacatttgcccactaaaactaaatttaaattcggagccatgatgaaatctggaatgttcctcaca
ctgtttgtgaacacagtcattaacattgtaatcgcaagcagagtgttgagagaacggctaaccggatcaccatgtgcagcattcattggaga
tgacaatatcgtgaaaggagtcaaatcggacaaattaatggcagacaggtgcgccacctggttgaatatggaagtcaagattatagatgct
gtggtgggcgagaaagcgccttatttctgtggagggtttattttgtgtgactccgtgaccggcacagcgtgccgtgtggcagaccccctaa
aaaggctgtttaagcttggcaaacctctggcagcagacgatgaacatgatgatgacaggagaagggcattgcatgaagagtcaacacgc
tggaaccgagtgggtattctttcagagctgtgcaaggcagtagaatcaaggtatgaaaccgtaggaacttccatcatagttatggccatgac
tactctagctagcagtgttaaatcattcagctacctgagaggggcccctataactctctacggctaacctgaatggactacgactctagaata
gtctttaattaaGCCACCATGGCTGGCGGTCCCCTGGTGAGAAAAATCTTCGTAGACGG
AGTGCCATTTGTGGTGTCTACCGGCTACCACTTTAGAGAGCTGGGGGTGCCCG
TCTACTCTTTCCTGCCTGGCGTCTACAGCGTCATTTATCTGTATCTCACCTTCT
ACCTGACAAATGATGTGAGTTTCCTGGCCCACATCCAGTGGATGGTCATGTTT
ACTCCTGGACTGATGTGGCTCAGCTACTTTATCGCGAGCTTTAGGCTCTTCGC
CCGCACGAGGTCCATGAATAATTTGGACAAGTCCGCTGGGTTCCCTTTCAACA
AATGGGGAAAGGCCCGGGCCACCACTCGTCAAGTTGTGAACGTCGTGACCAC
CAAAATTGCTCTGAAGACACTAGCCTGCTTCGTGCTGGCCGCCGTGTATAGAA
TCAACTGGATCACCGGAGGCCCATACCCTAATGCATCCTTCGATAACTTCAAG
TTTGTCTGTGACAACATTACCTACAACAAGGTGGAGAATATGACCCCACGCGA
TCTCGGCGCATGCATCGTGAGGGCTACCGCTACAATCCCCATCCAGGCCTCTC
TGCCATTCGGCTGGCTGATCGTGGGTGTGGCCCTGAACTTTAATAAAGACTTC
TATGACTTCGCCGTCTCTAAGGGGTTTTTCAAGGAGGGCTCATCTGGAGGCGA
TGGCAAGATGAAGGACCTGTCTCCTCGCTGGTACTTTTACTACCTGGGAACCG
GACCCAAACAGGGCGACGATTACGTCTATTTGCCTTACCCTGACCCTAGCAGA
ATCTTGGGCGCTGGATGCCACATCGATGCCTATAAGACATTTCCCCCTACGGA
GCCCAAGAAGGACAAAAAGAAGAAAGCAGACGAGTGTCGGAGCAAAAACCCC
CTGCTGTACGATGCCAATTACTTCCTGTGCTGGAGCCTCGCTACCGTGGCATA
TTTTAACATGGTATACATGCCCGCCTCTTGGGTGATGAGGATCATGACCTGGC
TTGACGGCAATTGCGATACATTGAAGGAAATTCTGGTGACCTATAACTGCTGC
GACGAGGCTTTTGAATACTACCACACAACCGATCCAAGTTTTCTTGGCAGATA
TATGAGCGCCTTGAACCACACTAAGAAGTGGAAGTACCCCCAGATCTCCAACG
AAAAGCAGGAGATTCTCGGAACCGTGTCCTGGAATCTGCGCGAGGATATTAAA
GACCTGCCTAAGGAGATCACTGTGGCCACCTCCAGAACCCTGTCCTATTACAA
GCTGGGAGCTTCACAGAGGGTGGCCGGAGATAGCGGGTTCGCCGCCTATAGC
CGGTACAGGATCGGCAACTGGCCTCAGATCGCCCAGTTTGCTCCCAGCGCCTC
AGCCTTCTTCGGAATGTCCCGCATCGGGATGGAAGTGACTCCTTCCGGAACTT
GGCTCACATACACCGGCGCTATTGATTGGTACGATTTCGTTGAGAATCCTGAC
ATCCTGCGGGTCTATGCCAACCTGGGCGCCTGCCCTCTCATTGCAGCCGTGAT
CACCAGGGAAGTTGGCTTTGTAGTGCCAGGATTACCGGGCACAATTCTGAGAA
CAACCAATGGAGACTTTCTGCACTTCCTTCCCCGGGTGTTCAGTGCTGTTGGA
AACATCTGCTACACCCCTTCTAAACTGATTGAGTACACAGATTTTGCCGGACC
CGGACCAGGCGCCAAATTTGTTGCTGCTTGGACACTGAAAGCTGCTGCTGGG
CCCGGACCAGGCCAGTACATCAAGGCCAACTCTAAGTTTATCGGCATCACCGA
ATTGGGACCTGGACCCGGCTAGTGAttcgaagggcccctataactctctacggctaacctgaatggactacga
catagtctagtccgccaagGCCACCATGTTTGTCTTCCTGGTCTTGCTGCCGCTGGTGAGCAGCCA
GTGCGTGAATTCACCACCCGCACCCAGCTTCCACCTGCCTACACTAACAGCTTCACCCG
AGGGGTGTATTACCCTGACAAGGTATTCCGGTCCTCCGTCCTCCATAGCACGCAGGACC
TTTTTCTGCCCTTCTTCTCAAATGTGACATGGTTCCATGCCATTCACGTGAGCGGCACGAA
TGGAACGAAGCGCTTTGcTAACCCCGTGCTGCCTTTCAATGACGGCGTCTACTTCGCCTC
CACTGAAAAGTCAAACATCATCCGGGGCTGGATCTTTGGCACCACTCTTGATTCAAAGAC
CCAGTCACTGCTGATTGTGAACAATGCTACAAACGTGGTTATCAAGGTGTGTGAGTTTCA
GTTCTGTAACGATCCATTTTTGGGAGTGTACTACCACAAGAACAACAAGTCCTGGATGGA
GTCTGAGTTCAGAGTGTATAGCTCTGCTAACAACTGCACCTTCGAGTACGTGTCCCAGCC
TTTCCTTATGGACCTGGAAGGCAAACAGGGCAATTTCAAAAACCTGAGAGAGTTCGTGTT
TAAGAACATTGACGGATACTTCAAAATTTATTCTAAGCACACACCAATTAACTTAGTGCGG
GgCCTACCCCAAGGCTTTAGCGCCCTAGAGCCCCTGGTTGACCTGCCCATTGGGATCAA
TATAACAAGGTTCCAAACTCTACATAtAAGTTATCTGACCCCAGGAGACAGCTCTAGTGGT
TGGACCGCCGGCGCAGCAGCCTACTATGTCGGGTACTTACAGCCACGCACGTTCCTTCT
GAAGTACAATGAGAACGGGACAATCACTGACGCAGTAGACTGTGCACTGGACCCGCTAA
GCGAGACTAAGTGCACACTTAAATCCTTCACGGTGGAGAAAGGCATTTATCAGACCTCTA
ACTTCAGGGTGCAGCCAACAGAAAGCATTGTGCGATTCCCAAATATTACTAATCTTTGCCC
TTTCGGGGAGGTCTTTAATGCAACTAGATTCGCATCAGTCTATGCGTGGAACCGCAAACG
CATTTCCAATTGTGTCGCAGACTACTCAGTGCTGTACAACTCTGCCTCTTTCAGTACGTTC
AAGTGTTACGGAGTGTCACCCACTAAACTGAACGACCTGTGCTTTACAAATGTCTACGCT
GACTCCTTCGTGATTAGGGGAGACGAGGTGAGACAAATTGCCCCCGGACAGACTGGGAA
tATTGCCGACTACAATTATAAGCTTCCTGATGATTTCACTGGCTGTGTTATTGCCTGGAATA
GTAACAATCTGGATAGCAAGGTGGGAGGCAACTATAACTACTTATATCGACTGTTTAGGA
AGAGTAATCTGAAACCATTTGAGCGGGATATTTCCACAGAAATTTACCAGGCCGGGAGCA
CACCATGTAATGGGGTGaAGGGATTTAATTGTTACTTCCCACTCCAGAGCTATGGTTTCCA
ACCCACCTATGGAGTGGGTTACCAGCCCTATAGAGTCGTGGTGCTTAGTTTTGAGCTGCT
TCACGCCCCAGCAACCGTCTGCGGTCCCAAAAAGTCGACCAATCTCGTGAAAAACAAATG
CGTAAACTTCAACTTTAACGGCTTAACAGGAACCGGCGTGCTCACCGAAAGCAACAAGAA
ATTCCTTCCATTTCAGCAATTCGGAAGGGACATCGCCGACACAACAGACGCGGTGAGGG
ACCCACAGACTCTGGAGATACTGGACATCACTCCTTGTTCGTTTGGGGGCGTCTCGGTCA
TCACACCCGGGACTAATACTAGTAATCAGGTAGCAGTTTTATATCAAGGCGTCAACTGTAC
CGAAGTACCTGTGGCCATACACGCTGATCAGCTAACGCCAACATGGCGAGTCTATTCCAC
CGGCTCTAACGTTTTTCAGACCAGGGCTGGGTGCCTGATAGGGGCAGAGCACGTCAATA
ATTCCTATGAGTGTGATATCCCCATAGGTGCGGGGATCTGTGCCAGCTATCAAACCCAAA
CCAATTCACCAgGGaGcGCAaGcTCTGTGGCTTCTCAGAGCATAATTGCATATACAATGTCA
CTGGGCGtTGAGAATAGCGTTGCATACTCTAATAACAGCATAGCCATTCCCACGAACTTTA
CTATCAGTGTGACAACCGAAATATTGCCAGTTTCGATGACCAAAACTAGCGTGGATTGCA
CGATGTACATCTGTGGAGACTCTACCGAATGCAGCAATCTGCTATTACAATATGGCAGCT
TCTGTACACAGTTAAATCGAGCCTTGACAGGCATCGCAGTGGAACAGGACAAAAATACTC
AAGAGGTGTTTGCACAGGTGAAGCAAATCTACAAAACGCCCCCCATTAAAGATTTTGGCG
GGTTCAATTTTTCACAAATTCTCCCCGACCCGTCTAAGCCGAGTAAGCGGTCCTTCATCG
AAGATCTGCTCTTTAACAAAGTAACCCTCGCCGATGCCGGCTTTATTAAGCAGTATGGCG
ACTGCCTGGGGGATATAGCCGCTCGTGACCTGATTTGCGCCCAGAAGTTCAATGGTCTG
ACCGTGTTGCCTCCTTTATTGACCGATGAAATGATTGCCCAGTACACTAGTGCCCTGCTG
GCCGGCACTATCACGTCTGGGTGGACCTTCGGAGCTGGTGCCGCCTTGCAGATACCTTT
TGCAATGCAGATGGCCTATAGGTTTAATGGTATCGGAGTGACTCAGAACGTACTGTACGA
GAACCAGAAGCTCATCGCTAATCAATTTAACTCCGCTATCGGAAAAATCCAGGACAGCCT
CTCTTCTACAGCTAGCGCTCTGGGCAAACTGCAGGATGTCGTTAATCAGAATGCCCAGGC
CCTGAACACCTTGGTTAAACAACTATCTTCCAACTTCGGGGCCATATCCAGTGTGTTGAAT
GATATTCTCTCCCGCTTGGATccacctGAAGCTGAGGTGCAGATCGATCGCTTGATCACCGG
CAGACTGCAGTCCCTCCAGACATATGTAACTCAGCAGCTGATTAGAGCCGCCGAGATAA
GGGCAAGTGCGAATCTGGCTGCCACCAAGATGAGCGAATGTGTATTGGGCCAGAGCAAA
CGAGTTGATTTTTGCGGTAAGGGGTATCATTTAATGTCTTTCCCTCAATCCGCACCTCATG
GCGTAGTTTTCCTGCATGTGACTTATGTCCCGGCTCAGGAGAAGAATTTTACCACAGCCC
CCGCGATCTGCCATGACGGAAAGGCCCACTTCCCCCGGGAAGGCGTGTTTGTATCCAAT
GGGACTCACTGGTTTGTCACTCAGCGAAATTTTTATGAACCACAGATCATCACCACTGACA
ACACATTTGTTAGTGGAAACTGCGATGTGGTCATCGGCATCGTGAATAACACTGTCTATG
ATCCACTGCAACCTGAACTGGATTCTTTTAAAGAGGAACTCGACAAGTATTTTAAAAACCA
CACTAGCCCTGACGTGGATCTCGGTGACATTTCTGGCATCAACGCTAGCGTAGTGAACAT
TCAGAAAGAGATAGATAGACTTAATGAGGTGGCCAAGAACCTCAACGAAAGTCTGATCGA
CCTCCAGGAACTGGGGAAATACGAGCAGTACATTAAATGGCCTTGGTACATATGGCTGG
GGTTCATTGCTGGGCTGATCGCAATAGTGATGGTGACCATAATGCTCTGTTGCATGACTA
GCTGCTGCAGCTGCCTGAAGGGCTGCTGTAGTTGTGGGTCATGTTGTAAGTTTGACGAA
GATGATAGCGAGCCTGTCCTTAAAGGAGTGAAGCTCCACTACACCTAGTAAttcgaacggccgt
atcacgcccaaacatttacagccgcggtgtcaaaaaccgcgtggacgtggttaacatccctgctgggaggatcagccgtaattattataatt
gGCTTGGTGCTGGCTACTATTgtggccaTGTACGTGCTGACCAACCAGAAACAtaattgaatac
agcagcaattggcaagctgCTTACATAGAACTCGCGGCGattggcatgccgccttaaaatttttattttattttttcttttctttt
ccgaatcggattttgtttttaatatttcaaaaaaaaaaaaaaaaaaaaaaaaaaaaaaaaaaaaaaaaaaaaaaaaaaaaaaaaaaaaaaa
aaaaaaaaaaaaaaaaa
SAM-SGP1-TCE5-SGP2-CTSpikeGF2P Nucleotide Sequence in vitro transcription
template DNA sequence (SEQ ID NO: 93): (NT 1-17: T7 promoter; NT 62-7543: VEEV non-
structural protein coding region; NT 7518-7560: SGP1; NT 7582-7587 and 9541-9546: Kozak
sequence; NT 7588-9474: TCE5 cassette; NT 9480-9540: SGP2; NT 9547-13368: CTSpike
Furin-2P)
taatacgactcactataatgggcggcgcatgagagaagcccagaccaattacctacccaaaatggagaaagttcacgttgacatcgagga
agacagcccattcctcagagctttgcagcggagcttcccgcagtttgaggtagaagccaagcaggtcactgataatgaccatgctaatgcc
agagcgttttcgcatctggcttcaaaactgatcgaaacggaggtggacccatccgacacgatccttgacattggaagtgcgcccgcccgc
agaatgtattctaagcacaagtatcattgtatctgtccgatgagatgtgcggaagatccggacagattgtataagtatgcaactaagctgaag
aaaaactgtaaggaaataactgataaggaattggacaagaaaatgaaggagctcgccgccgtcatgagcgaccctgacctggaaactga
gactatgtgcctccacgacgacgagtcgtgtcgctacgaagggcaagtcgctgtttaccaggatgtatacgcggttgacggaccgacaag
tctctatcaccaagccaataagggagttagagtcgcctactggataggctttgacaccaccccttttatgtttaagaacttggctggagcatat
ccatcatactctaccaactgggccgacgaaaccgtgttaacggctcgtaacataggcctatgcagctctgacgttatggagcggtcacgta
gagggatgtccattcttagaaagaagtatttgaaaccatccaacaatgttctattctctgttggctcgaccatctaccacgagaagagggactt
actgaggagctggcacctgccgtctgtatttcacttacgtggcaagcaaaattacacatgtcggtgtgagactatagttagttgcgacgggt
acgtcgttaaaagaatagctatcagtccaggcctgtatgggaagccttcaggctatgctgctacgatgcaccgcgagggattcttgtgctgc
aaagtgacagacacattgaacggggagagggtctcttttcccgtgtgcacgtatgtgccagctacattgtgtgaccaaatgactggcatact
ggcaacagatgtcagtgcggacgacgcgcaaaaactgctggttgggctcaaccagcgtatagtcgtcaacggtcgcacccagagaaac
accaataccatgaaaaattaccttttgcccgtagtggcccaggcatttgctaggtgggcaaaggaatataaggaagatcaagaagatgaaa
ggccactaggactacgagatagacagttagtcatggggtgttgttgggcttttagaaggcacaagataacatctatttataagcgcccggat
acccaaaccatcatcaaagtgaacagcgatttccactcattcgtgctgcccaggataggcagtaacacattggagatcgggctgagaaca
agaatcaggaaaatgttagaggagcacaaggagccgtcacctctcattaccgccgaggacgtacaagaagctaagtgcgcagccgatg
aggctaaggaggtgcgtgaagccgaggagttgcgcgcagctctaccacctttggcagctgatgttgaggagcccactctggaagccgat
gtcgacttgatgttacaagaggctggggccggctcagtggagacacctcgtggcttgataaaggttaccagctacgctggcgaggacaa
gatcggctcttacgctgtgctttctccgcaggctgtactcaagagtgaaaaattatcttgcatccaccctctcgctgaacaagtcatagtgata
acacactctggccgaaaagggcgttatgccgtggaaccataccatggtaaagtagtggtgccagagggacatgcaatacccgtccagga
ctttcaagctctgagtgaaagtgccaccattgtgtacaacgaacgtgagttcgtaaacaggtacctgcaccatattgccacacatggagga
gcgctgaacactgatgaagaatattacaaaactgtcaagcccagcgagcacgacggcgaatacctgtacgacatcgacaggaaacagt
gcgtcaagaaagaactagtcactgggctagggctcacaggcgagctggtggatcctcccttccatgaattcgcctacgagagtctgagaa
cacgaccagccgctccttaccaagtaccaaccataggggtgtatggcgtgccaggatcaggcaagtctggcatcattaaaagcgcagtc
accaaaaaagatctagtggtgagcgccaagaaagaaaactgtgcagaaattataagggacgtcaagaaaatgaaagggctggacgtca
atgccagaactgtggactcagtgctcttgaatggatgcaaacaccccgtagagaccctgtatattgacgaagcttttgcttgtcatgcaggta
ctctcagagcgctcatagccattataagacctaaaaaggcagtgctctgcggggatcccaaacagtgcggtttttttaacatgatgtgcctga
aagtgcattttaaccacgagatttgcacacaagtcttccacaaaagcatctctcgccgttgcactaaatctgtgacttcggtcgtctcaacctt
gttttacgacaaaaaaatgagaacgacgaatccgaaagagactaagattgtgattgacactaccggcagtaccaaacctaagcaggacg
atctcattctcacttgtttcagagggtgggtgaagcagttgcaaatagattacaaaggcaacgaaataatgacggcagctgcctctcaaggg
ctgacccgtaaaggtgtgtatgccgttcggtacaaggtgaatgaaaatcctctgtacgcacccacctcagaacatgtgaacgtcctactgac
ccgcacggaggaccgcatcgtgtggaaaacactagccggcgacccatggataaaaacactgactgccaagtaccctgggaatttcactg
ccacgatagaggagtggcaagcagagcatgatgccatcatgaggcacatcttggagagaccggaccctaccgacgtcttccagaataa
ggcaaacgtgtgttgggccaaggctttagtgccggtgctgaagaccgctggcatagacatgaccactgaacaatggaacactgtggatta
ttttgaaacggacaaagctcactcagcagagatagtattgaaccaactatgcgtgaggttctttggactcgatctggactccggtctattttct
gcacccactgttccgttatccattaggaataatcactgggataactccccgtcgcctaacatgtacgggctgaataaagaagtggtccgtca
gctctctcgcaggtacccacaactgcctcgggcagttgccactggaagagtctatgacatgaacactggtacactgcgcaattatgatccg
cgcataaacctagtacctgtaaacagaagactgcctcatgctttagtcctccaccataatgaacacccacagagtgacttttcttcattcgtca
gcaaattgaagggcagaactgtcctggtggtcggggaaaagttgtccgtcccaggcaaaatggttgactggttgtcagaccggcctgag
gctaccttcagagctcggctggatttaggcatcccaggtgatgtgcccaaatatgacataatatttgttaatgtgaggaccccatataaatacc
atcactatcagcagtgtgaagaccatgccattaagcttagcatgttgaccaagaaagcttgtctgcatctgaatcccggcggaacctgtgtc
agcataggttatggttacgctgacagggccagcgaaagcatcattggtgctatagcgcggcagttcaagttttcccgggtatgcaaaccga
aatcctcacttgaagagacggaagttctgtttgtattcattgggtacgatcgcaaggcccgtacgcacaatccttacaagctttcatcaacctt
gaccaacatttatacaggttccagactccacgaagccggatgtgcaccctcatatcatgtggtgcgaggggatattgccacggccaccga
aggagtgattataaatgctgctaacagcaaaggacaacctggcggaggggtgtgcggagcgctgtataagaaattcccggaaagcttcg
atttacagccgatcgaagtaggaaaagcgcgactggtcaaaggtgcagctaaacatatcattcatgccgtaggaccaaacttcaacaaagt
ttcggaggttgaaggtgacaaacagttggcagaggcttatgagtccatcgctaagattgtcaacgataacaattacaagtcagtagcgattc
cactgttgtccaccggcatcttttccgggaacaaagatcgactaacccaatcattgaaccatttgctgacagctttagacaccact gatgcag
atgtagccatatactgcagggacaagaaatgggaaatgactctcaaggaagcagtggctaggagagaagcagtggaggagatatgcat
atccgacgactcttcagtgacagaacctgatgcagagctggtgagggtgcatccgaagagttctttggctggaaggaagggctacagca
caagcgatggcaaaactttctcatatttggaagggaccaagtttcaccaggcggccaaggatatagcagaaattaatgccatgtggcccgt
tgcaacggaggccaatgagcaggtatgcatgtatatcctcggagaaagcatgagcagtattaggtcgaaatgccccgtcgaagagtcgg
aagcctccacaccacctagcacgctgccttgcttgtgcatccatgccatgactccagaaagagtacagcgcctaaaagcctcacgtccag
aacaaattactgtgtgctcatcctttccattgccgaagtatagaatcactggtgtgcagaagatccaatgctcccagcctatattgttctcacc
gaaagtgcctgcgtatattcatccaaggaagtatctcgtggaaacaccaccggtagacgagactccggagccatcggcagagaaccaatc
cacagaggggacacctgaacaaccaccacttataaccgaggatgagaccaggactagaacgcctgagccgatcatcatcgaagagga
agaagaggatagcataagtttgctgtcagatggcccgacccaccaggtgctgcaagtcgaggcagacattcacgggccgccctctgtat
ctagctcatcctggtccattcctcatgcatccgactttgatgtggacagtttatccatacttgacaccctggagggagctagcgtgaccagcg
gggcaacgtcagccgagactaactcttacttcgcaaagagtatggagtttctggcgcgaccggtgcctgcgcctcgaacagtattcagga
accctccacatcccgctccgcgcacaagaacaccgtcacttgcacccagcagggcctgctcgagaaccagcctagtttccaccccgcca
ggcgtgaatagggtgatcactagagaggagctcgaggcgcttaccccgtcacgcactcctagcaggtcggtctcgagaaccagcctggt
ctccaacccgccaggcgtaaatagggtgattacaagagaggagtttgaggcgttcgtagcacaacaacaatgacggtttgatgcgggtgc
atacatcttttcctccgacaccggtcaagggcatttacaacaaaaatcagtaaggcaaacggtgctatccgaagtggtgttggagaggacc
gaattggagatttcgtatgccccgcgcctcgaccaagaaaaagaagaattactacgcaagaaattacagttaaatcccacacctgctaaca
gaagcagataccagtccaggaaggtggagaacatgaaagccataacagctagacgtattctgcaaggcctagggcattatttgaaggca
gaaggaaaagtggagtgctaccgaaccctgcatcctgttcctttgtattcatctagtgtgaaccgtgccttttcaagccccaaggtcgcagtg
gaagcctgtaacgccatgttgaaagagaactttccgactgtggcttcttactgtattattccagagtacgatgcctatttggacatggttgacg
gagcttcatgctgcttagacactgccagtttttgccctgcaaagctgcgcagctttccaaagaaacactcctatttggaacccacaatacgat
cggcagtgccttcagcgatccagaacacgctccagaacgtcctggcagctgccacaaaaagaaattgcaatgtcacgcaaatgagagaa
ttgcccgtattggattcggcggcctttaatgtggaatgcttcaagaaatatgcgtgtaataatgaatattgggaaacgtttaaagaaaacccca
tcaggcttactgaagaaaacgtggtaaattacattaccaaattaaaaggaccaaaagctgctgctctttttgcgaagacacataatttgaatat
gttgcaggacataccaatggacaggtttgtaatggacttaaagagagacgtgaaagtgactccaggaacaaaacatactgaagaacggc
ccaaggtacaggtgatccaggctgccgatccgctagcaacagcgtatctgtgcggaatccaccgagagctggttaggagattaaatgcg
gtcctgcttccgaacattcatacactgtttgatatgtcggctgaagactttgacgctattatagccgagcacttccagcctggggattgtgttct
ggaaactgacatcgcgtcgtttgataaaagtgaggacgacgccatggctctgaccgcgttaatgattctggaagacttaggtgtggacgca
gagctgttgacgctgattgaggcggctttcggcgaaatttcatcaatacatttgcccactaaaactaaatttaaattcggagccatgatgaaat
ctggaatgttcctcacactgtttgtgaacacagtcattaacattgtaatcgcaagcagagtgttgagagaacggctaaccggatcaccatgtg
cagcattcattggagatgacaatatcgtgaaaggagtcaaatcggacaaattaatggcagacaggtgcgccacctggttgaatatggaagt
caagattatagatgctgtggtgggcgagaaagcgccttatttctgtggagggtttattttgtgtgactccgtgaccggcacagcgtgccgtgt
ggcagaccccctaaaaaggctgtttaagcttggcaaacctctggcagcagacgatgaacatgatgatgacaggagaagggcattgcatg
aagagtcaacacgctggaaccgagtgggtattctttcagagctgtgcaaggcagtagaatcaaggtatgaaaccgtaggaacttccatcat
agttatggccatgactactctagctagcagtgttaaatcattcagctacctgagaggggcccctataactctctacggctaacctgaatggac
tacgactctagaatagtctttaattaagccaccatggctggcgaggcccccttcctttacctgtacgcccttgtgtatttcctgcagagcatca
attttgtgagaatcatcatgaggctgtggctttgctggaaatgtaggagcaagaaccccctgttgtatgacgccaactactttctgtgttggca
caccaatctcgccgtgttccagagtgcctctaagatcattacactgaaaaagcggtggcagcttgcactttctaagggagtgcatttcgtttgc
aacctgctcctggtgacactcaagcagggggaaatcaaagacgccacccctagcgacttcgttagagccactgccacaatcccaatcca
ggcttccctgcctttcggctggcttatcgtgggtgtggcactgttggctgtgcggagaccacagggactgcctaataatacagctagctggt
ttaccgctctgacacagcatggcaaagaagacctcaagttccctcgcggtcagggggtgcctattaacactaatagctctccagacgacca
aattgggtattacaggcgcgccacaagacggatcatggcctgcttagtggggctgatgtggctatcctattttattgctagctttcgcctgaa
gaaggacaagaagaagaaagctgatgagacccaggcactgccccagcgccaaaagaagcagcagacagtcacactgctccctgctg
cagacctggatgacttcagcaagcagctgcaggggaactttggcgaccaggagctgattagacaggggactgactataagcattggcct
cagattgctcagttcgccccaagtgcatccgccttcttcgggatgtcacgaataggaatggaagtgaccccttctgggacatggttgacata
caccggagcaatcaagattacctccggggacggtaccacgtctcctattagcgaacacgattatcagatagggggatatactgagaagtg
ggagtccggcgtcaaaaagatgagtgggaaaggccagcagcaacaaggccaaacagttactaaaaagtctgccgcagaagctagtaa
aaagcctcgccagaagcggacagccaccaaagcttacaatgtgactcaggccttcggccgccgggggcctgaacagaccctcttgtgg
cccgttaccctcgcatgtttcgtgcttgcagctgtgtacaggatcaattggtttaaggatcaggttatcctgttgaacaaacatatagatgcct
ataagacattcccacccaccgagccaaagaaagataagaaaaagaaaactagtcctgcaaggatggccggcaatggaggagacgcagc
cttagccctgctcttactcgacaggctgaaccaacttgagtctaaaatgagcggtaaagggcagaagatgaaggatctgtccccaaggtg
gtatttctactatctgggcaccggccctgaggattgtgtcgtcctccactcatacttcactagcgattattaccagctgtatagtacacaatta
tctaccgacacaggcgtcgagcacgtgaccttctttatatacaataagatcgtggatgaaccagaggagcatgtgcagatccacactattgat
ggctctagcgggggcatcatctgggtggcaacagaaggagccctcaacaccccaaaggaccatatcggcaccaggaatccagccaac
aatgccgccattgttctgcagctccctcagggcactactctccctaaaggcttctatgctgagggacccggaccaggcgccaaatttgttgc
tgcttggacactgaaagctgctgctgggcccggaccaggccagtacatcaaggccaactctaagtttatcggcatcaccgaattgggacc
tggacccggctagtgattgggcccctataactctctacggctaacctgaatggactacgacatagtctagtccgccaaggccaccatgtttg
tcttcctggtcttgctgccgctggtgagcagccagtgcgtgaatctcaccacccgcacccagcttccacctgcctacactaacagcttcacc
cgaggggtgtattaccctgacaaggtattccggtcctccgtcctccatagcacgcaggacctttttctgcccttcttctcaaatgtgacatggt
tccatgccattcacgtgagcggcacgaatggaacgaagcgctttgataaccccgtgctgcctttcaatgacggcgtctacttcgcctccact
gaaaagtcaaacatcatccggggctggatctttggcaccactcttgattcaaagacccagtcactgctgattgtgaacaatgctacaaacgt
ggttatcaaggtgtgtgagtttcagttctgtaacgatccatttttgggagtgtactaccacaagaacaacaagtcctggatggagtctgagttc
agagtgtatagctctgctaacaactgcaccttcgagtacgtgtcccagcctttccttatggacctggaaggcaaacagggcaatttcaaaaa
cctgagagagttcgtgtttaagaacattgacggatacttcaaaatttattctaagcacacaccaattaacttagtgcgggacctaccccaagg
ctttagcgccctagagcccctggttgacctgcccattgggatcaatataacaaggttccaaactctactggctctgcatagaagttatctgac
cccaggagacagctctagtggttggaccgccggcgcagcagcctactatgtcgggtacttacagccacgcacgttccttctgaagtacaa
tgagaacgggacaatcactgacgcagtagactgtgcactggacccgctaagcgagactaagtgcacacttaaatccttcacggtggaga
aaggcatttatcagacctctaacttcagggtgcagccaacagaaagcattgtgcgattcccaaatattactaatctttgccctttcggggaggt
ctttaatgcaactagattcgcatcagtctatgcgtggaaccgcaaacgcatttccaattgtgtcgcagactactcagtgctgtacaactctgcc
tctttcagtacgttcaagtgttacggagtgtcacccactaaactgaacgacctgtgctttacaaatgtctacgctgactccttcgtgattaggg
gagacgaggtgagacaaattgcccccggacagactgggaagattgccgactacaattataagcttcctgatgatttcactggctgtgttattg
cctggaatagtaacaatctggatagcaaggtgggaggcaactataactacttatatcgactgtttaggaagagtaatctgaaaccatttgagc
gggatatttccacagaaatttaccaggccgggagcacaccatgtaatggggggagggatttaattgttacttcccactccagagctatggtt
tccaacccaccaatggagtgggttaccagccctatagagtcgtggtgcttagttttgagctgcttcacgccccagcaaccgtctgcggtccc
aaaaagtcgaccaatctcgtgaaaaacaaatgcgtaaacttcaactttaacggcttaacaggaaccggcgtgctcaccgaaagcaacaag
aaattccttccatttcagcaattcggaagggacatcgccgacacaacagacgcggtgagggacccacagactctggagatactggacatc
actccttgttcgtttgggggcgtctcggtcatcacacccgggactaatactagtaatcaggtagcagttttatatcaaggcgtcaactgtaccg
aagtacctgtggccatacacgctgatcagctaacgccaacatggcgagtctattccaccggctctaacgtttttcagaccagggctgggtg
cctgataggggcagagcacgtcaataattcctatgagtgtgatatccccataggtgcggggatctgtgccagctatcaaacccaaaccaat
tcaccagggagcgcaagctctgtggcttctcagagcataattgcatatacaatgtcactgggcgctgagaatagcgttgcatactctaataa
cagcatagccattcccacgaactttactatcagtgtgacaaccgaaatattgccagtttcgatgaccaaaactagcgtggattgcacgatgta
catctgtggagactctaccgaatgcagcaatctgctattacaatatggcagcttctgtacacagttaaatcgagccttgacaggcatcgcagt
ggaacaggacaaaaatactcaagaggtgtttgcacaggtgaagcaaatctacaaaacgccccccattaaagattttgggggttcaattttt
cacaaattctccccgacccgtctaagccgagtaagcggtccttcatcgaagatctgctctttaacaaagtaaccctcgccgatgccggcttt
attaagcagtatggcgactgcctgggggatatagccgctcgtgacctgatttgcgcccagaagttcaatggtctgaccgtgttgcctccttta
ttgaccgatgaaatgattgcccagtacactagtgccctgctggccggcactatcacgtctgggtggaccttcggagctggtgccgccttgc
agataccttttgcaatgcagatggcctataggtttaatggtatcggagtgactcagaacgtactgtacgagaaccagaagctcatcgctaatc
aatttaactccgctatcggaaaaatccaggacagcctctcttctacagctagcgctctgggcaaactgcaggatgtcgttaatcagaatgcc
caggccctgaacaccttggttaaacaactatcttccaacttcggggccatatccagtgtgttgaatgatattctctcccgcttggatccacctg
aagctgaggtgcagatcgatcgcttgatcaccggcagactgcagtccctccagacatatgtaactcagcagctgattagagccgccgaga
taagggcaagtgcgaatctggctgccaccaagatgagcgaatgtgtattgggccagagcaaacgagttgatttttgcggtaaggggtatc
atttaatgtctttccctcaatccgcacctcatggcgtagttttcctgcatgtgacttatgtcccggctcaggagaagaattttaccacagcccc
cgcgatctgccatgacggaaaggcccacttcccccgggaaggcgtgtttgtatccaatgggactcactggtttgtcactcagcgaaatttttat
gaaccacagatcatcaccactgacaacacatttgttagtggaaactgcgatgtggtcatcggcatcgtgaataacactgtctatgatccactg
caacctgaactggattcttttaaagaggaactcgacaagtattttaaaaaccacactagccctgacgtggatctcggtgacatttctggcatc
aacgctagcgtagtgaacattcagaaagagatagatagacttaatgaggtggccaagaacctcaacgaaagtctgatcgacctccaggaa
ctggggaaatacgagcagtacattaaatggccttggtacatatggctggggttcattgctgggctgatcgcaatagtgatggtgaccataat
gctctgttgcatgactagctgctgcagctgcctgaagggctgctgtagttgtgggtcatgttgtaagtttgacgaagatgatagcgagcctgt
ccttaaaggagtgaagctccactacacctagtaacgaacggccgtatcacgcccaaacatttacagccgcggtgtcaaaaaccgcgtgg
acgtggttaacatccctgctgggaggatcagccgtaattattataattggcttggtgctggctactattgtggccatgtacgtgctgaccaacc
agaaacataattgaatacagcagcaattggcaagctgcttacatagaactcgcggcgattggcatgccgccttaaaatttttattttatttttt
cttttcttttccgaatcggattttgtttttaatatttcaaaaaaaaaaaaaaaaaaaaaaaaaaaaaaaaaaaaaaaaaaaaaaaaaaaaaaaa
aaaaaaaaaaaaaaaaaaaaaaaatacgtagtttaaac
SAM-SGP1-TCE5-SGP2-CTSpikeGF2P Nucleotide Sequence samRNA (SEQ ID
NO: 27983); GRT-910: samRNA produced from SEQ ID NO: 93
atgggcggcgcatgagagaagcccagaccaattacctacccaaaatggagaaagttcacgttgacatcgaggaagacagcccattcctc
agagctttgcagcggagcttcccgcagtttgaggtagaagccaagcaggtcactgataatgaccatgctaatgccagagcgttttcgcatc
tggcttcaaaactgatcgaaacggaggtggacccatccgacacgatccttgacattggaagtgcgcccgcccgcagaatgtattctaagc
acaagtatcattgtatctgtccgatgagatgtgcggaagatccggacagattgtataagtatgcaactaagctgaagaaaaactgtaaggaa
ataactgataaggaattggacaagaaaatgaaggagctcgccgccgtcatgagcgaccctgacctggaaactgagactatgtgcctcca
cgacgacgagtcgtgtcgctacgaagggcaagtcgctgtttaccaggatgtatacgcggttgacggaccgacaagtctctatcaccaagc
caataagggagttagagtcgcctactggataggctttgacaccaccccttttatgtttaagaacttggctggagcatatccatcatactctacc
aactgggccgacgaaaccgtgttaacggctcgtaacataggcctatgcagctctgacgttatggagcggtcacgtagagggatgtccatt
cttagaaagaagtatttgaaaccatccaacaatgttctattctctgttggctcgaccatctaccacgagaagagggacttactgaggagctgg
cacctgccgtctgtatttcacttacgtggcaagcaaaattacacatgtcggtgtgagactatagttagttgcgacgggtacgtcgttaaaaga
atagctatcagtccaggcctgtatgggaagccttcaggctatgctgctacgatgcaccgcgagggattcttgtgctgcaaagtgacagaca
cattgaacggggagagggtctcttttcccgtgtgcacgtatgtgccagctacattgtgtgaccaaatgactggcatactggcaacagatgtc
agtgcggacgacgcgcaaaaactgctggttgggctcaaccagcgtatagtcgtcaacggtcgcacccagagaaacaccaataccatga
aaaattaccttttgcccgtagtggcccaggcatttgctaggtgggcaaaggaatataaggaagatcaagaagatgaaaggccactaggac
tacgagatagacagttagtcatggggtgttgttgggcttttagaaggcacaagataacatctatttataagcgcccggatacccaaaccatca
tcaaagtgaacagcgatttccactcattcgtgctgcccaggataggcagtaacacattggagatcgggctgagaacaagaatcaggaaaa
tgttagaggagcacaaggagccgtcacctctcattaccgccgaggacgtacaagaagctaagtgcgcagccgatgaggctaaggaggt
gcgtgaagccgaggagttgcgcgcagctctaccacctttggcagctgatgttgaggagcccactctggaagccgatgtcgacttgatgtta
caagaggctggggccggctcagtggagacacctcgtggcttgataaaggttaccagctacgctggcgaggacaagatcggctcttacgc
tgtgctttctccgcaggctgtactcaagagtgaaaaattatcttgcatccaccctctcgctgaacaagtcatagtgataacacactctggccg
aaaagggcgttatgccgtggaaccataccatggtaaagtagtggtgccagagggacatgcaatacccgtccaggactttcaagctctgag
tgaaagtgccaccattgtgtacaacgaacgtgagttcgtaaacaggtacctgcaccatattgccacacatggaggagcgctgaacactgat
gaagaatattacaaaactgtcaagcccagcgagcacgacggcgaatacctgtacgacatcgacaggaaacagtgcgtcaagaaagaac
tagtcactgggctagggctcacaggcgagctggtggatcctcccttccatgaattcgcctacgagagtctgagaacacgaccagccgctc
cttaccaagtaccaaccataggggtgtatggcgtgccaggatcaggcaagtctggcatcattaaaagcgcagtcaccaaaaaagatctag
tggtgagcgccaagaaagaaaactgtgcagaaattataagggacgtcaagaaaatgaaagggctggacgtcaatgccagaactgtgga
ctcagtgctcttgaatggatgcaaacaccccgtagagaccctgtatattgacgaagcttttgcttgtcatgcaggtactctcagagcgctcat
agccattataagacctaaaaaggcagtgctctgcggggatcccaaacagtgcggtttttttaacatgatgtgcctgaaagtgcattttaacca
cgagatttgcacacaagtcttccacaaaagcatctctcgccgttgcactaaatctgtgacttcggtcgtctcaaccttgttttacgacaaaaaa
atgagaacgacgaatccgaaagagactaagattgtgattgacactaccggcagtaccaaacctaagcaggacgatctcattctcacttgttt
cagagggtgggtgaagcagttgcaaatagattacaaaggcaacgaaataatgacggcagctgcctctcaagggctgacccgtaaaggt
gtgtatgccgttcggtacaaggtgaatgaaaatcctctgtacgcacccacctcagaacatgtgaacgtcctactgacccgcacggaggac
cgcatcgtgtggaaaacactagccggcgacccatggataaaaacactgactgccaagtaccctgggaatttcactgccacgatagagga
gtggcaagcagagcatgatgccatcatgaggcacatcttggagagaccggaccctaccgacgtcttccagaataaggcaaacgtgtgtt
gggccaaggctttagtgccggtgctgaagaccgctggcatagacatgaccactgaacaatggaacactgtggattattttgaaacggaca
aagctcactcagcagagatagtattgaaccaactatgcgtgaggttctttggactcgatctggactccggtctattttctgcacccactgttcc
gttatccattaggaataatcactgggataactccccgtcgcctaacatgtacgggctgaataaagaagtggtccgtcagctctctcgcaggt
acccacaactgcctcgggcagttgccactggaagagtctatgacatgaacactggtacactgcgcaattatgatccgcgcataaacctagt
acctgtaaacagaagactgcctcatgctttagtcctccaccataatgaacacccacagagtgacttttcttcattcgtcagcaaattgaaggg
cagaactgtcctggtggtcggggaaaagttgtccgtcccaggcaaaatggttgactggttgtcagaccggcctgaggctaccttcagagct
cggctggatttaggcatcccaggtgatgtgcccaaatatgacataatatttgttaatgtgaggaccccatataaataccatcactatcagcagt
gtgaagaccatgccattaagcttagcatgttgaccaagaaagcttgtctgcatctgaatcccggcggaacctgtgtcagcataggttatggtt
acgctgacagggccagcgaaagcatcattggtgctatagcgcggcagttcaagttttcccgggtatgcaaaccgaaatcctcacttgaag
agacggaagttctgtttgtattcattgggtacgatcgcaaggcccgtacgcacaatccttacaagctttcatcaaccttgaccaacatttatac
aggttccagactccacgaagccggatgtgcaccctcatatcatgtggtgcgaggggatattgccacggccaccgaaggagtgattataaa
tgctgctaacagcaaaggacaacctggcggaggggtgtgcggagcgctgtataagaaattcccggaaagcttcgatttacagccgatcg
aagtaggaaaagcgcgactggtcaaaggtgcagctaaacatatcattcatgccgtaggaccaaacttcaacaaagtttcggaggttgaag
gtgacaaacagttggcagaggcttatgagtccatcgctaagattgtcaacgataacaattacaagtcagtagcgattccactgttgtccacc
ggcatcttttccgggaacaaagatcgactaacccaatcattgaaccatttgctgacagctttagacaccactgatgcagatgtagccatatac
tgcagggacaagaaatgggaaatgactctcaaggaagcagtggctaggagagaagcagtggaggagatatgcatatccgacgactctt
cagtgacagaacctgatgcagagctggtgagggtgcatccgaagagttctttggctggaaggaagggctacagcacaagcgatggcaa
aactttctcatatttggaagggaccaagtttcaccaggcggccaaggatatagcagaaattaatgccatgtggcccgttgcaacggaggcc
aatgagcaggtatgcatgtatatcctcggagaaagcatgagcagtattaggtcgaaatgccccgtcgaagagtcggaagcctccacacca
cctagcacgctgccttgcttgtgcatccatgccatgactccagaaagagtacagcgcctaaaagcctcacgtccagaacaaattactgtgt
gctcatcctttccattgccgaagtatagaatcactggtgtgcagaagatccaatgctcccagcctatattgttctcaccgaaagtgcctgcgta
tattcatccaaggaagtatctcgtggaaacaccaccggtagacgagactccggagccatcggcagagaaccaatccacagaggggaca
cctgaacaaccaccacttataaccgaggatgagaccaggactagaacgcctgagccgatcatcatcgaagaggaagaagaggatagca
taagtttgctgtcagatggcccgacccaccaggtgctgcaagtcgaggcagacattcacgggccgccctctgtatctagctcatcctggtc
cattcctcatgcatccgactttgatgtggacagtttatccatacttgacaccctggagggagctagcgtgaccagcggggcaacgtcagcc
gagactaactcttacttcgcaaagagtatggagtttctggcgcgaccggtgcctgcgcctcgaacagtattcaggaaccctccacatcccg
ctccgcgcacaagaacaccgtcacttgcacccagcagggcctgctcgagaaccagcctagtttccaccccgccaggcgtgaatagggt
gatcactagagaggagctcgaggcgcttaccccgtcacgcactcctagcaggtcggtctcgagaaccagcctggtctccaacccgccag
gcgtaaatagggtgattacaagagaggagtttgaggcgttcgtagcacaacaacaatgacggtttgatgcgggtgcatacatcttttcctcc
gacaccggtcaagggcatttacaacaaaaatcagtaaggcaaacggtgctatccgaagtggtgttggagaggaccgaattggagatttcg
tatgccccgcgcctcgaccaagaaaaagaagaattactacgcaagaaattacagttaaatcccacacctgctaacagaagcagataccag
tccaggaaggtggagaacatgaaagccataacagctagacgtattctgcaaggcctagggcattatttgaaggcagaaggaaaagtgga
gtgctaccgaaccctgcatcctgttcctttgtattcatctagtgtgaaccgtgccttttcaagccccaaggtcgcagtggaagcctgtaacgc
catgttgaaagagaactttccgactgtggcttcttactgtattattccagagtacgatgcctatttggacatggttgacggagcttcatgctgc
ttagacactgccagtttttgccctgcaaagctgcgcagctttccaaagaaacactcctatttggaacccacaatacgatcggcagtgccttcag
cgatccagaacacgctccagaacgtcctggcagctgccacaaaaagaaattgcaatgtcacgcaaatgagagaattgcccgtattggatt
cggcggcctttaatgtggaatgcttcaagaaatatgcgtgtaataatgaatattgggaaacgtttaaagaaaaccccatcaggcttactgaag
aaaacgtggtaaattacattaccaaattaaaaggaccaaaagctgctgctctttttgcgaagacacataatttgaatatgttgcaggacatacc
aatggacaggtttgtaatggacttaaagagagacgtgaaagtgactccaggaacaaaacatactgaagaacggcccaaggtacaggtga
tccaggctgccgatccgctagcaacagcgtatctgtgcggaatccaccgagagctggttaggagattaaatgcggtcctgcttccgaacat
tcatacactgtttgatatgtcggctgaagactttgacgctattatagccgagcacttccagcctggggattgtgttctggaaactgacatcgcg
tcgtttgataaaagtgaggacgacgccatggctctgaccgcgttaatgattctggaagacttaggtgtggacgcagagctgttgacgctgat
tgaggcggctttcggcgaaatttcatcaatacatttgcccactaaaactaaatttaaattcggagccatgatgaaatctggaatgttcctcaca
ctgtttgtgaacacagtcattaacattgtaatcgcaagcagagtgttgagagaacggctaaccggatcaccatgtgcagcattcattggaga
tgacaatatcgtgaaaggagtcaaatcggacaaattaatggcagacaggtgcgccacctggttgaatatggaagtcaagattatagatgct
gtggtgggcgagaaagcgccttatttctgtggagggtttattttgtgtgactccgtgaccggcacagcgtgccgtgtggcagaccccctaa
aaaggctgtttaagcttggcaaacctctggcagcagacgatgaacatgatgatgacaggagaagggcattgcatgaagagtcaacacgc
tggaaccgagtgggtattctttcagagctgtgcaaggcagtagaatcaaggtatgaaaccgtaggaacttccatcatagttatggccatgac
tactctagctagcagtgttaaatcattcagctacctgagaggggcccctataactctctacggctaacctgaatggactacgactctagaata
gtctttaattaagccaccatggctggcgaggcccccttcctttacctgtacgcccttgtgtatttcctgcagagcatcaattttgtgagaatca
tcatgaggctgtggctttgctggaaatgtaggagcaagaaccccctgttgtatgacgccaactactttctgtgttggcacaccaatctcgccgt
gttccagagtgcctctaagatcattacactgaaaaagcggtggcagcttgcactttctaagggagtgcatttcgtttgcaacctgctcctggtg
acactcaagcagggggaaatcaaagacgccacccctagcgacttcgttagagccactgccacaatcccaatccaggcttccctgcctttcg
gctggcttatcgtgggtgtggcactgttggctgtgcggagaccacagggactgcctaataatacagctagctggtttaccgctctgacaca
gcatggcaaagaagacctcaagttccctcgcggtcagggggtgcctattaacactaatagctctccagacgaccaaattgggtattacagg
cgcgccacaagacggatcatggcctgcttagtggggctgatgtggctatcctattttattgctagctttcgcctgaagaaggacaagaagaa
gaaagctgatgagacccaggcactgccccagcgccaaaagaagcagcagacagtcacactgctccctgctgcagacctggatgacttc
agcaagcagctgcaggggaactttggcgaccaggagctgattagacaggggactgactataagcattggcctcagattgctcagttcgcc
ccaagtgcatccgccttcttcgggatgtcacgaataggaatggaagtgaccccttctgggacatggttgacatacaccggagcaatcaag
attacctccggggacggtaccacgtctcctattagcgaacacgattatcagatagggggatatactgagaagtgggagtccggcgtcaaa
aagatgagtgggaaaggccagcagcaacaaggccaaacagttactaaaaagtctgccgcagaagctagtaaaaagcctcgccagaag
cggacagccaccaaagcttacaatgtgactcaggccttcggccgccgggggcctgaacagaccctcttgtggcccgttaccctcgcatgt
ttcgtgcttgcagctgtgtacaggatcaattggtttaaggatcaggttatcctgttgaacaaacatatagatgcctataagacattcccaccca
ccgagccaaagaaagataagaaaaagaaaactagtcctgcaaggatggccggcaatggaggagacgcagccttagccctgctcttact
cgacaggctgaaccaacttgagtctaaaatgagcggtaaagggcagaagatgaaggatctgtccccaaggtggtatttctactatctgggc
accggccctgaggattgtgtcgtcctccactcatacttcactagcgattattaccagctgtatagtacacaattatctaccgacacaggcgtc
gagcacgtgaccttctttatatacaataagatcgtggatgaaccagaggagcatgtgcagatccacactattgatggctctagcgggggca
tcatctgggtggcaacagaaggagccctcaacaccccaaaggaccatatcggcaccaggaatccagccaacaatgccgccattgttctg
cagctccctcagggcactactctccctaaaggcttctatgctgagggacccggaccaggcgccaaatttgttgctgcttggacactgaaag
ctgctgctgggcccggaccaggccagtacatcaaggccaactctaagtttatcggcatcaccgaattgggacctggacccggctagtgat
tgggcccctataactctctacggctaacctgaatggactacgacatagtctagtccgccaaggccaccatgtttgtcttcctggtcttgctgcc
gctggtgagcagccagtgcgtgaatctcaccacccgcacccagcttccacctgcctacactaacagcttcacccgaggggtgtattaccct
gacaaggtattccggtcctccgtcctccatagcacgcaggacctttttctgcccttcttctcaaatgtgacatggttccatgccattcacgtga
gcggcacgaatggaacgaagcgctttgataaccccgtgctgcctttcaatgacggcgtctacttcgcctccactgaaaagtcaaacatcat
ccggggctggatctttggcaccactcttgattcaaagacccagtcactgctgattgtgaacaatgctacaaacgtggttatcaaggtgtgtga
gtttcagttctgtaacgatccatttttgggagtgtactaccacaagaacaacaagtcctggatggagtctgagttcagagtgtatagctctgct
aacaactgcaccttcgagtacgtgtcccagcctttccttatggacctggaaggcaaacagggcaatttcaaaaacctgagagagttcgtgtt
taagaacattgacggatacttcaaaatttattctaagcacacaccaattaacttagtgcgggacctaccccaaggctttagcgccctagagcc
cctggttgacctgcccattgggatcaatataacaaggttccaaactctactggctctgcatagaagttatctgaccccaggagacagctcta
gtggttggaccgccggcgcagcagcctactatgtcgggtacttacagccacgcacgttccttctgaagtacaatgagaacgggacaatca
ctgacgcagtagactgtgcactggacccgctaagcgagactaagtgcacacttaaatccttcacggtggagaaaggcatttatcagacctc
taacttcagggtgcagccaacagaaagcattgtgcgattcccaaatattactaatctttgccctttcggggaggtctttaatgcaactagattc
gcatcagtctatgcgtggaaccgcaaacgcatttccaattgtgtcgcagactactcagtgctgtacaactctgcctctttcagtacgttcaagt
gttacggagtgtcacccactaaactgaacgacctgtgctttacaaatgtctacgctgactccttcgtgattaggggagacgaggtgagacaa
attgcccccggacagactgggaagattgccgactacaattataagcttcctgatgatttcactggctgtgttattgcctggaatagtaacaatc
tggatagcaaggtgggaggcaactataactacttatatcgactgtttaggaagagtaatctgaaaccatttgagcgggatatttccacagaa
atttaccaggccgggagcacaccatgtaatggggggagggatttaattgttacttcccactccagagctatggtttccaacccaccaatgg
agtgggttaccagccctatagagtcgtggtgcttagttttgagctgcttcacgccccagcaaccgtctgcggtcccaaaaagtcgaccaatc
tcgtgaaaaacaaatgcgtaaacttcaactttaacggcttaacaggaaccggcgtgctcaccgaaagcaacaagaaattccttccatttcag
caattcggaagggacatcgccgacacaacagacgcggtgagggacccacagactctggagatactggacatcactccttgttcgtttgg
gggcgtctcggtcatcacacccgggactaatactagtaatcaggtagcagttttatatcaaggcgtcaactgtaccgaagtacctgtggcca
tacacgctgatcagctaacgccaacatggcgagtctattccaccggctctaacgtttttcagaccagggctgggtgcctgataggggcaga
gcacgtcaataattcctatgagtgtgatatccccataggtgcggggatctgtgccagctatcaaacccaaaccaattcaccagggagcgca
agctctgtggcttctcagagcataattgcatatacaatgtcactgggcgctgagaatagcgttgcatactctaataacagcatagccattccc
acgaactttactatcagtgtgacaaccgaaatattgccagtttcgatgaccaaaactagcgtggattgcacgatgtacatctgtggagactct
accgaatgcagcaatctgctattacaatatggcagcttctgtacacagttaaatcgagccttgacaggcatcgcagtggaacaggacaaaa
atactcaagaggtgtttgcacaggtgaagcaaatctacaaaacgccccccattaaagattttggcgggttcaatttttcacaaattctccccga
cccgtctaagccgagtaagcggtccttcatcgaagatctgctctttaacaaagtaaccctcgccgatgccggctttattaagcagtatggcg
actgcctgggggatatagccgctcgtgacctgatttgcgcccagaagttcaatggtctgaccgtgttgcctcctttattgaccgatgaaatga
ttgcccagtacactagtgccctgctggccggcactatcacgtctgggtggaccttcggagctggtgccgccttgcagataccttttgcaatg
cagatggcctataggtttaatggtatcggagtgactcagaacgtactgtacgagaaccagaagctcatcgctaatcaatttaactccgctatc
ggaaaaatccaggacagcctctcttctacagctagcgctctgggcaaactgcaggatgtcgttaatcagaatgcccaggccctgaacacc
ttggttaaacaactatcttccaacttcggggccatatccagtgtgttgaatgatattctctcccgcttggatccacctgaagctgaggtgcaga
tcgatcgcttgatcaccggcagactgcagtccctccagacatatgtaactcagcagctgattagagccgccgagataagggcaagtgcga
atctggctgccaccaagatgagcgaatgtgtattgggccagagcaaacgagttgatttttgcggtaaggggtatcatttaatgtctttccctca
atccgcacctcatggcgtagttttcctgcatgtgacttatgtcccggctcaggagaagaattttaccacagcccccgcgatctgccatgacg
gaaaggcccacttcccccgggaaggcgtgtttgtatccaatgggactcactggtttgtcactcagcgaaatttttatgaaccacagatcatca
ccactgacaacacatttgttagtggaaactgcgatgtggtcatcggcatcgtgaataacactgtctatgatccactgcaacctgaactggatt
cttttaaagaggaactcgacaagtattttaaaaaccacactagccctgacgtggatctcggtgacatttctggcatcaacgctagcgtagtga
acattcagaaagagatagatagacttaatgaggtggccaagaacctcaacgaaagtctgatcgacctccaggaactggggaaatacgag
cagtacattaaatggccttggtacatatggctggggttcattgctgggctgatcgcaatagtgatggtgaccataatgctctgttgcatgacta
gctgctgcagctgcctgaagggctgctgtagttgtgggtcatgttgtaagtttgacgaagatgatagcgagcctgtccttaaaggagtgaag
ctccactacacctagtaacgaacggccgtatcacgcccaaacatttacagccgcggtgtcaaaaaccgcgtggacgtggttaacatccct
gctgggaggatcagccgtaattattataattggcttggtgctggctactattgtggccatgtacgtgctgaccaaccagaaacataattgaat
acagcagcaattggcaagctgcttacatagaactcgcggcgattggcatgccgccttaaaatttttattttattttttcttttcttttccgaat
cggattttgtttttaatatttcaaaaaaaaaaaaaaaaaaaaaaaaaaaaaaaaaaaaaaaaaaaaaaaaaaaaaaaaaaaaaaaaaaaaaaaa
aaaaaaaa

### REFERENCES

1. Desrichard, A., Snyder, A. & Chan, T. A. Cancer Neoantigens and Applications for Immunotherapy. Clin. Cancer Res. Off, J. Am. Assoc. Cancer Res. (2015). doi:10.1158/1078-0432.CCR-14-3175
2. Schumacher, T. N. & Schreiber, R. D. Neoantigens in cancer immunotherapy. Science 348, 69-74 (2015).
3. Gubin, M. M., Artyomov, M. N., Mardis, E. R. & Schreiber, R. D. Tumor neoantigens: building a framework for personalized cancer immunotherapy. J. Clin. Invest. 125, 3413-3421 (2015).
4. Rizvi, N. A. et al. Cancer immunology. Mutational landscape determines sensitivity to PD-1 blockade in non-small cell lung cancer. Science 348, 124-128 (2015).
5. Snyder, A. et al. Genetic basis for clinical response to CTLA-4 blockade in melanoma. N. Engl. J. Med. 371, 2189-2199 (2014).
6. Carreno, B. M. et al. Cancer immunotherapy. A dendritic cell vaccine increases the breadth and diversity of melanoma neoantigen-specific T cells. Science 348, 803-808 (2015).
7. Tran, E. et al. Cancer immunotherapy based on mutation-specific CD4+ T cells in a patient with epithelial cancer. Science 344, 641-645 (2014).
8. Hacohen, N. & Wu, C. J.-Y. United States Patent Application: 20110293637—COMPOSITIONS AND METHODS OF IDENTIFYING TUMOR SPECIFIC NEOANTIGENS. (A1). at ppftl.uspto.gov/netacgi/nph-Parser?Sect1=PTO1&Sect2=HITOFF&d=PG01&p=1&u=/netahtml/PTO/srchnum.html&r=1&f=G&1=50&s1=20110293637.PGNR.>
9. Lundegaard, C., Hoof, I., Lund, O. & Nielsen, M. State of the art and challenges in sequence based T-cell epitope prediction. Immunome Res. 6 Suppl 2, S3 (2010).
10. Yadav, M. et al. Predicting immunogenic tumour mutations by combining mass spectrometry and exome sequencing. Nature 515, 572-576 (2014).
11. Bassani-Sternberg, M., Pletscher-Frankild, S., Jensen, L. J. & Mann, M. Mass spectrometry of human leukocyte antigen class I peptidomes reveals strong effects of protein abundance and turnover on antigen presentation. Mol. Cell. Proteomics MCP 14, 658-673 (2015).
12. Van Allen, E. M. et al. Genomic correlates of response to CTLA-4 blockade in metastatic melanoma. Science 350, 207-211 (2015).
13. Yoshida, K. & Ogawa, S. Splicing factor mutations and cancer. Wiley Interdiscip. Rev. RNA 5, 445-459 (2014).
14. Cancer Genome Atlas Research Network. Comprehensive molecular profiling of lung adenocarcinoma. Nature 511, 543-550 (2014).
15. Rajasagi, M. et al. Systematic identification of personal tumor-specific neoantigens in chronic lymphocytic leukemia. Blood 124, 453-462 (2014).
16. Downing, S. R. et al. U.S. patent application 0,120,208,706—OPTIMIZATION OF MULTIGENE ANALYSIS OF TUMOR SAMPLES. (A1). at appft1.uspto.gov/netacgi/nph-Parser?Sect1=PTO1&Sect2=HITOFF&d=PG01&p=1&u=/netahtml/PTO/srchnum.html&r=1&f=G&l=50&s1=20120208706.PGNR.
17. Target Capture for NextGen Sequencing—IDT. at www.idtdna.com/pages/products/nextgen/target-capture
18. Shukla, S. A. et al. Comprehensive analysis of cancer-associated somatic mutations in class I HLA genes. Nat. Biotechnol. 33, 1152-1158 (2015).
19. Cieslik, M. et al. The use of exome capture RNA-seq for highly degraded RNA with application to clinical cancer sequencing. Genome Res. 25, 1372-1381 (2015).
20. Bodini, M. et al. The hidden genomic landscape of acute myeloid leukemia: subclonal structure revealed by undetected mutations. Blood 125, 600-605 (2015).
21. Saunders, C. T. et al. Strelka: accurate somatic small-variant calling from sequenced tumor-normal sample pairs. Bioinforma. Oxf. Engl. 28, 1811-1817 (2012).
22. Cibulskis, K. et al. Sensitive detection of somatic point mutations in impure and heterogeneous cancer samples. Nat. Biotechnol. 31, 213-219 (2013).
23. Wilkerson, M. D. et al. Integrated RNA and DNA sequencing improves mutation detection in low purity tumors. Nucleic Acids Res. 42, e107 (2014).
24. Mose, L. E., Wilkerson, M. D., Hayes, D. N., Perou, C. M. & Parker, J. S. ABRA: improved coding indel detection via assembly-based realignment. Bioinforma. Oxf Engl. 30, 2813-2815 (2014).
25. Ye, K., Schulz, M. H., Long, Q., Apweiler, R. & Ning, Z. Pindel: a pattern growth approach to detect break points of large deletions and medium sized insertions from paired-end short reads. Bioinforma. Oxf Engl. 25, 2865-2871 (2009).
26. Lam, H. Y. K. et al. Nucleotide-resolution analysis of structural variants using BreakSeq and a breakpoint library. Nat. Biotechnol. 28, 47-55 (2010).
27. Frampton, G. M. et al. Development and validation of a clinical cancer genomic profiling test based on massively parallel DNA sequencing. Nat. Biotechnol. 31, 1023-1031 (2013).
28. Boegel, S. et al. HLA typing from RNA-Seq sequence reads. Genome Med. 4, 102 (2012).
29. Liu, C. et al. ATHLATES: accurate typing of human leukocyte antigen through exome sequencing. Nucleic Acids Res. 41, e142 (2013).
30. Mayor, N. P. et al. HLA Typing for the Next Generation. PloS One 10, e0127153 (2015).
31. Roy, C. K., Olson, S., Graveley, B. R., Zamore, P. D. & Moore, M. J. Assessing long-distance RNA sequence connectivity via RNA-templated DNA-DNA ligation. eLife 4, (2015).
32. Song, L. & Florea, L. CLASS: constrained transcript assembly of RNA-seq reads. BMC Bioinformatics 14 Suppl 5, S14 (2013).
33. Maretty, L., Sibbesen, J. A. & Krogh, A. Bayesian transcriptome assembly. Genome Biol. 15, 501 (2014).
34. Pertea, M. et al. StringTie enables improved reconstruction of a transcriptome from RNA-seq reads. Nat. Biotechnol. 33, 290-295 (2015).
35. Roberts, A., Pimentel, H., Trapnell, C. & Pachter, L. Identification of novel transcripts in annotated genomes using RNA-Seq. Bioinforma. Oxf. Engl. (2011). doi:10.1093/bioinformatics/btr355
36. Vitting-Seerup, K., Porse, B. T., Sandelin, A. & Waage, J. spliceR: an R package for classification of alternative splicing and prediction of coding potential from RNA-seq data. BMC Bioinformatics 15, 81 (2014).
37. Rivas, M. A. et al. Human genomics. Effect of predicted protein-truncating genetic variants on the human transcriptome. Science 348, 666-669 (2015).
38. Skelly, D. A., Johansson, M., Madeoy, J., Wakefield, J. & Akey, J. M. A powerful and flexible statistical framework for testing hypotheses of allele-specific gene expression from RNA-seq data. Genome Res. 21, 1728-1737 (2011).
39. Anders, S., Pyl, P. T. & Huber, W. HTSeq—a Python framework to work with high-throughput sequencing data. Bioinforma. Oxf Engl. 31, 166-169 (2015).
40. Furney, S. J. et al. SF3B1 mutations are associated with alternative splicing in uveal melanoma. Cancer Discov. (2013). doi:10.1158/2159-8290.CD-13-0330
41. Zhou, Q. et al. A chemical genetics approach for the functional assessment of novel cancer genes. Cancer Res. (2015). doi:10.1158/0008-5472.CAN-14-2930
42. Maguire, S. L. et al. SF3B1 mutations constitute a novel therapeutic target in breast cancer. J. Pathol. 235, 571-580 (2015).
43. Carithers, L. J. et al. A Novel Approach to High-Quality Postmortem Tissue Procurement: The GTEx Project. Biopreservation Biobanking 13, 311-319 (2015).
44. Xu, G. et al. RNA CoMPASS: a dual approach for pathogen and host transcriptome analysis of RNA-seq datasets. PloS One 9, e89445 (2014).
45. Andreatta, M. & Nielsen, M. Gapped sequence alignment using artificial neural networks: application to the MHC class I system. Bioinforma. Oxf Engl. (2015). doi:10.1093/bioinformatics/btv639
46. Jorgensen, K. W., Rasmussen, M., Buus, S. & Nielsen, M. NetMHCstab—predicting stability of peptide-MHC-I complexes; impacts for cytotoxic T lymphocyte epitope discovery. Immunology 141, 18-26 (2014).
47. Larsen, M. V. et al. An integrative approach to CTL epitope prediction: a combined algorithm integrating MHC class I binding, TAP transport efficiency, and proteasomal cleavage predictions. Eur. J. Immunol. 35, 2295-2303 (2005).
48. Nielsen, M., Lundegaard, C., Lund, O. & Keşmir, C. The role of the proteasome in generating cytotoxic T-cell epitopes: insights obtained from improved predictions of proteasomal cleavage. Immunogenetics 57, 33-41 (2005).
49. Boisvert, F.-M. et al. A Quantitative Spatial Proteomics Analysis of Proteome Turnover in Human Cells. Mol. Cell. Proteomics 11, M111.011429-M111.011429 (2012).
50. Duan, F. et al. Genomic and bioinformatic profiling of mutational neoepitopes reveals new rules to predict anticancer immunogenicity. J. Exp. Med. 211, 2231-2248 (2014).
51. Janeway's Immunobiology: 9780815345312: Medicine & Health Science Books @Amazon.com. at www.amazon.com/Janeways-Immunobiology-Kenneth-Murphy/dp/0815345313
52. Calis, J. J. A. et al. Properties of MHC Class I Presented Peptides That Enhance Immunogenicity. PLoS Comput. Biol. 9, e1003266 (2013).
53. Zhang, J. et al. Intratumor heterogeneity in localized lung adenocarcinomas delineated by multiregion sequencing. Science 346, 256-259 (2014)
54. Walter, M. J. et al. Clonal architecture of secondary acute myeloid leukemia. N. Engl. J. Med. 366, 1090-1098 (2012).
55. Hunt D F, Henderson R A, Shabanowitz J, Sakaguchi K, Michel H, Sevilir N, Cox A L, Appella E, Engelhard V H. Characterization of peptides bound to the class I MHC molecule HLA-A2.1 by mass spectrometry. Science 1992. 255: 1261-1263.
56. Zarling A L, Polefrone J M, Evans A M, Mikesh L M, Shabanowitz J, Lewis S T, Engelhard V H, Hunt D F. Identification of class I MHC-associated phosphopeptides as targets for cancer immunotherapy. Proc Natl Acad Sci USA. 2006 Oct. 3; 103(40):14889-94.
57. Bassani-Sternberg M, Pletscher-Frankild S, Jensen L J, Mann M. Mass spectrometry of human leukocyte antigen class I peptidomes reveals strong effects of protein abundance and turnover on antigen presentation. Mol Cell Proteomics. 2015 March; 14(3):658-73. doi: 10.1074/mcp.M114.042812.
58. Abelin J G, Trantham P D, Penny S A, Patterson A M, Ward S T, Hildebrand W H, Cobbold M, Bai D L, Shabanowitz J, Hunt D F. Complementary IMAC enrichment methods for HLA-associated phosphopeptide identification by mass spectrometry. Nat Protoc. 2015 September; 10(9):1308-18. doi: 10.1038/nprot.2015.086. Epub 2015 Aug. 6
59. Barnstable C J, Bodmer W F, Brown G, Galfre G, Milstein C, Williams A F, Ziegler A. Production of monoclonal antibodies to group A erythrocytes, HLA and other human cell surface antigens-new tools for genetic analysis. Cell. 1978 May; 14(1):9-20.
60. Goldman J M, Hibbin J, Kearney L, Orchard K, Th'ng K H. HLA-DR monoclonal antibodies inhibit the proliferation of normal and chronic granulocytic leukaemia myeloid progenitor cells. Br J Haematol. 1982 November; 52(3):411-20.
61. Eng J K, Jahan T A, Hoopmann M R. Comet: an open-source MS/MS sequence database search tool. Proteomics. 2013 January; 13(1):22-4. doi: 10.1002/pmic.201200439. Epub 2012 Dec. 4.
62. Eng J K, Hoopmann M R, Jahan T A, Egertson J D, Noble W S, MacCoss M J. A deeper look into Comet—implementation and features. J Am Soc Mass Spectrom. 2015 November; 26(11):1865-74. doi: 10.1007/s13361-015-1179-x. Epub 2015 Jun. 27.
63. Lukas Käll, Jesse Canterbury, Jason Weston, William Stafford Noble and Michael J. MacCoss. Semi-supervised learning for peptide identification from shotgun proteomics datasets. Nature Methods 4:923-925, November 2007
64. Lukas Käll, John D. Storey, Michael J. MacCoss and William Stafford Noble. Assigning confidence measures to peptides identified by tandem mass spectrometry. Journal of Proteome Research, 7(1):29-34, January 2008
65. Lukas Käll, John D. Storey and William Stafford Noble. Nonparametric estimation of posterior error probabilities associated with peptides identified by tandem mass spectrometry. Bioinformatics, 24(16):i42-i48, August 2008
66. Kinney R M, B J Johnson, V L Brown, D W Trent. Nucleotide Sequence of the 26 S mRNA of the Virulent Trinidad Donkey Strain of Venezuelan Equine Encephalitis Virus and Deduced Sequence of the Encoded Structural Proteins. Virology 152 (2), 400-413. 1986 Jul. 30.
67. Jill E Slansky, Frédérique M Rattis, Lisa F Boyd, Tarek Fahmy, Elizabeth M Jaffee, Jonathan P Schneck, David H Margulies, Drew M Pardoll. Enhanced Antigen-Specific Antitumor Immunity with Altered Peptide Ligands that Stabilize the MHC-Peptide-TCR Complex. Immunity, Volume 13, Issue 4, 1 Oct. 2000, Pages 529-538.
68. A Y Huang, P H Gulden, A S Woods, M C Thomas, C D Tong, W Wang, V H Engelhard, G Pasternack, R Cotter, D Hunt, D M Pardoll, and E M Jaffee. The immunodominant major histocompatibility complex class I-restricted antigen of a murine colon tumor derives from an endogenous retroviral gene product. Proc Natl Acad Sci USA.; 93(18): 9730-9735, 1996 Sep. 3.
69. JOHNSON, BARBARA J. B., RICHARD M. KINNEY, CRYSTLE L. KOST AND DENNIS W. TRENT. Molecular Determinants of Alphavirus Neurovirulence: Nucleotide and Deduced Protein Sequence Changes during Attenuation of Venezuelan Equine Encephalitis Virus. J Gen Virol 67:1951-1960, 1986.
70. Aarnoudse, C. A., Krüse, M., Konopitzky, R., Brouwenstijn, N., and Schrier, P. I. (2002). TCR reconstitution in Jurkat reporter cells facilitates the identification of novel tumor antigens by cDNA expression cloning. Int J Cancer 99, 7-13.
71. Alexander, J., Sidney, J., Southwood, S., Ruppert, J., Oseroff, C., Maewal, A., Snoke, K., Serra, H. M., Kubo, R. T., and Sette, A. (1994). Development of high potency universal DR-restricted helper epitopes by modification of high affinity DR-blocking peptides. Immunity 1, 751-761.
72. Banu, N., Chia, A., Ho, Z. Z., Garcia, A. T., Paravasivam, K., Grotenbreg, G. M., Bertoletti, A., and Gehring, A. J. (2014). Building and optimizing a virus-specific T cell receptor library for targeted immunotherapy in viral infections. Scientific Reports 4, 4166.
73. Cornet, S., Miconnet, I., Menez, J., Lemonnier, F., and Kosmatopoulos, K. (2006). Optimal organization of a polypeptide-based candidate cancer vaccine composed of cryptic tumor peptides with enhanced immunogenicity. Vaccine 24, 2102-2109.
74. Depla, E., van der Aa, A., Livingston, B. D., Crimi, C., Allosery, K., de Brabandere, V., Krakover, J., Murthy, S., Huang, M., Power, S., et al. (2008). Rational design of a multiepitope vaccine encoding T-lymphocyte epitopes for treatment of chronic hepatitis B virus infections. Journal of Virology 82, 435-450.
75. Ishioka, G. Y., Fikes, J., Hermanson, G., Livingston, B., Crimi, C., Qin, M., del Guercio, M. F., Oseroff, C., Dahlberg, C., Alexander, J., et al. (1999). Utilization of MHC class I transgenic mice for development of minigene DNA vaccines encoding multiple HLA-restricted CTL epitopes. J Immunol 162, 3915-3925.
76. Janetzki, S., Price, L., Schroeder, H., Britten, C. M., Welters, M. J. P., and Hoos, A. (2015). Guidelines for the automated evaluation of Elispot assays. Nat Protoc 10, 1098-1115.
77. Lyons, G. E., Moore, T., Brasic, N., Li, M., Roszkowski, J. J., and Nishimura, M. I. (2006). Influence of human CD8 on antigen recognition by T-cell receptor-transduced cells. Cancer Res 66, 11455-11461.
78. Nagai, K., Ochi, T., Fujiwara, H., An, J., Shirakata, T., Mineno, J., Kuzushima, K., Shiku, H., Melenhorst, J. J., Gostick, E., et al. (2012). Aurora kinase A-specific T-cell receptor gene transfer redirects T lymphocytes to display effective antileukemia reactivity. Blood 119, 368-376.
79. Panina-Bordignon, P., Tan, A., Termijtelen, A., Demotz, S., Corradin, G., and Lanzavecchia, A. (1989). Universally immunogenic T cell epitopes: promiscuous binding to human MHC class II and promiscuous recognition by T cells. Eur J Immunol 19, 2237-2242.
80. Vitiello, A., Marchesini, D., Furze, J., Sherman, L. A., and Chesnut, R. W. (1991). Analysis of the HLA-restricted influenza-specific cytotoxic T lymphocyte response in transgenic mice carrying a chimeric human-mouse class I major histocompatibility complex. J Exp Med 173, 1007-1015.
81. Yachi, P. P., Ampudia, J., Zal, T., and Gascoigne, N. R. J. (2006). Altered peptide ligands induce delayed CD8-T cell receptor interaction—a role for CD8 in distinguishing antigen quality. Immunity 25, 203-211.
82. Pushko P, Parker M, Ludwig G V, Davis N L, Johnston R E, Smith J F. Replicon-helper systems from attenuated Venezuelan equine encephalitis virus: expression of heterologous genes in vitro and immunization against heterologous pathogens in vivo. Virology. 1997 Dec. 22; 239(2):389-401.
83. Strauss, J H and E G Strauss. The alphaviruses: gene expression, replication, and evolution. Microbiol Rev. 1994 September; 58(3): 491-562.
84. Rhême C, Ehrengruber M U, Grandgirard D. Alphaviral cytotoxicity and its implication in vector development. Exp Physiol. 2005 January; 90(1):45-52. Epub 2004 Nov. 12.
85. Riley, Michael K. II, and Wilfred Vermerris. Recent Advances in Nanomaterials for Gene Delivery—A Review. Nanomaterials 2017, 7(5), 94.
86. Frolov I, Hardy R, Rice C M. Cis-acting RNA elements at the 5′ end of Sindbis virus genome RNA regulate minus- and plus-strand RNA synthesis. RNA. 2001 November; 7(11):1638-51.
87. Jose J, Snyder J E, Kuhn R J. A structural and functional perspective of alphavirus replication and assembly. Future Microbiol. 2009 September; 4(7):837-56.
88. Bo Li and C. olin N. Dewey. RSEM: accurate transcript quantification from RNA-Seq data with or without a reference genome. BMC Bioinformatics, 12:323, August 2011
89. Hillary Pearson, Tariq Daouda, Diana Paola Granados, Chantal Durette, Eric Bonneil, Mathieu Courcelles, Anja Rodenbrock, Jean-Philippe Laverdure, Caroline Côté, Sylvie Mader, Sébastien Lemieux, Pierre Thibault, and Claude Perreault. MHC class I-associated peptides derive from selective regions of the human genome. The Journal of Clinical Investigation, 2016,
90. Juliane Liepe, Fabio Marino, John Sidney, Anita Jeko, Daniel E. Bunting, Alessandro Sette, Peter M. Kloetzel, Michael P. H. Stumpf, Albert J. R. Heck, Michele Mishto. A large fraction of HLA class I ligands are proteasome-generated spliced peptides. Science, 21, October 2016.
91. Mommen G P., Marino, F., Meiring H D., Poelen, M C., van Gaans-van den Brink, J A., Mohammed S., Heck A J., and van Els C A. Sampling From the Proteome to the Human Leukocyte Antigen-D R (HLA-D R) Ligandome Proceeds Via High Specificity. Mol Cell Proteomics 15(4): 1412-1423, April 2016.
92. Sebastian Kreiter, Mathias Vormehr, Niels van de Roemer, Mustafa Diken, Martin Löwer, Jan Diekmann, Sebastian Boegel, Barbara Schrërs, Fulvia Vascotto, John C. Castle, Arbel D. Tadmor, Stephen P. Schoenberger, Christoph Huber, Özlem Tureci, and Ugur Sahin. Mutant MHC class II epitopes drive therapeutic immune responses to caner. Nature 520, 692-696, April 2015.
93. Tran E., Turcotte S., Gros A., Robbins P. F., Lu Y. C., Dudley M. E., Wunderlich J. R., Somerville R. P., Hogan K., Hinrichs C. S., Parkhurst M. R., Yang J. C., Rosenberg S. A. Cancer immunotherapy based on mutation-specific CD4+ T cells in a patient with epithelial cancer. Science 344(6184) 641-645, May 2014.
94. Andreatta M., Karosiene E., Rasmussen M., Stryhn A., Buus S., Nielsen M. Accurate pan-specific prediction of peptide-MHC class II binding affinity with improved binding core identification. Immunogenetics 67(11-12) 641-650, November 2015.
95. Nielsen, M., Lund, O. NN-align. An artificial neural network-based alignment algorithm for MHC class I I peptide binding prediction. BMC Bioinformatics 10:296, September 2009.
96. Nielsen, M., Lundegaard, C., Lund, O. Prediction of MHC class II binding affinity using SMM-align, a novel stabilization matrix alignment method. BMC Bioinformatics 8:238, July 2007.
97. Zhang, J., et al. PEAKS D B: de novo sequencing assisted database search for sensitive and accurate peptide identification. Molecular & Cellular Proteomics. 11(4):1-8. Jan. 2, 2012.
98. Jensen, Kamilla Kjaergaard, et al. “Improved Methods for Prediting Peptide Binding Affinity to MHC Class II Molecules.” Immunology, 2018, doi:10.1111/imm.12889.
99. Carter, S. L., Cibulskis, K., Helman, E., McKenna, A., Shen, H., Zack, T., Laird, P. W., Onofrio, R. C., Winckler, W., Weir, B. A., et al. (2012). Absolute quantification of somatic DNA alterations in human cancer. Nat. Biotechnol. 30, 413-421
100. McGranahan, N., Rosenthal, R., Hiley, C. T., Rowan, A. J., Watkins, T. B. K., Wilson, G. A., Birkbak, N. J., Veeriah, S., Van Loo, P., Herrero, J., et al. (2017). Allele-Specific HLA Loss and Immune Escape in Lung Cancer Evolution. Cell 171, 1259-1271.e11.
101. Shukla, S. A., Rooney, M. S., Rajasagi, M., Tiao, G., Dixon, P. M., Lawrence, M. S., Stevens, J., Lane, W. J., Dellagatta, J. L., Steelman, S., et al. (2015). Comprehensive analysis of cancer-associated somatic mutations in class I HLA genes. Nat. Biotechnol. 33, 1152-1158.
102. Van Loo, P., Nordgard, S. H., Lingjærde, O. C., Russnes, H. G., Rye, I. H., Sun, W., Weigman, V. J., Marynen, P., Zetterberg, A., Naume, B., et al. (2010). Allele-specific copy number analysis of tumors. Proc. Natl. Acad. Sci. U.S.A 107, 16910-16915.
103. Van Loo, P., Nordgard, S. H., Lingjorde, O. C., Russnes, H. G., Rye, I. H., Sun, W., Weigman, V. J., Marynen, P., Zetterberg, A., Naume, B., et al. (2010). Allele-specific copy number analysis of tumors. Proc. Natl. Acad. Sci. U.S.A 107, 16910-16915.

1. The composition of claim 40, wherein the antigen expression system comprises a self-amplifying alphavirus-based expression system,
wherein the self-amplifying alphavirus-based expression system comprises:
(A) one or more vectors, wherein the one or more vectors comprises:
(a) an RNA alphavirus backbone, wherein the RNA alphavirus backbone comprises:
(i) at least one promoter nucleotide sequence, and
(ii) at least one polyadenylation (poly(A)) sequence; and
(b) the antigen cassette, wherein the antigen cassette is inserted into the vector backbone,
(ii) optionally, a second promoter nucleotide sequence operably linked to the SARS-CoV-2 derived nucleic acid sequence; and
(iii) optionally, at least one MHC class II epitope-encoding nucleic acid sequence;
(iv) optionally, at least one nucleic acid sequence encoding a GPGPG amino acid linker sequence (SEQ ID NO:56); and
(v) optionally, at least one second poly(A) sequence, wherein the second poly(A) sequence is a native poly(A) sequence or an exogenous poly(A) sequence to the vector backbone, optionally wherein the exogenous poly(A) sequence comprises an SV40 poly(A) signal sequence or a Bovine Growth Hormone (BGH) poly(A) signal sequence; and
(B) a lipid-nanoparticle (LNP), wherein the LNP encapsulates the self-amplifying alphavirus-based expression system, and
wherein the composition comprises at least 10 μg of each of the one or more vectors;
optionally wherein the composition is further formulated in a pharmaceutical composition comprising a pharmaceutically acceptable carrier.

2. The composition of claim 1, wherein the composition for delivery of the self-amplifying alphavirus-based expression system comprises:
(a) at least 30 μg of each of the one or more vectors;
(b) 30 μg or less of each of the one or more vectors;
(c) between 10-30 μg or between 10-100 μg of each of the one or more vectors;
(d) at least 10 μg total of the one or more vectors combined;
(e) at least 30 μg total of the one or more vectors combined;
(f) 30 μg or less total of the one or more vectors combined; or
(g) between 10-30 μg or between 10-100 μg total of the one or more vectors combined.

3-8. (canceled)

9. The composition of claim 1, wherein:
(a) the one or more vectors of the self-amplifying alphavirus-based expression system is at a concentration of 1 mg/mL;
(b) the RNA alphavirus backbone comprises one or more elements obtained from the sequence of SEQ ID NO:3 or SEQ ID NO:5, optionally wherein the one or more elements are selected from the group consisting of the sequences necessary for nonstructural protein-mediated amplification, the 26S promoter nucleotide sequence, the poly(A) sequence, and the nsP1-4 genes of the sequence set forth in SEQ ID NO:3 or SEQ ID NO:5, optionally the RNA alphavirus backbone comprises the sequence set forth in the sequences selected from the group consisting of SEQ ID NOs:6-9; and/or
(c) the self-amplifying alphavirus-based expression system comprises a vector selected from the group of sequences consisting of: SEQ ID NO: 27983, SEQ ID NO:27981, SEQ ID NO:27982, SEQ ID NO: 27976, and SEQ ID NO: 27976 with Spike encoding sequences substituted with the sequence set forth in SEQ ID NO:27980.

10. (canceled)

11. (canceled)

12. A method for stimulating an immune response in a subject, the method comprising administering to the subject the composition for delivery of the self-amplifying alphavirus-based expression system of claim 1; optionally wherein the method comprises administering at least two doses of the composition for delivery of the self-amplifying alphavirus-based expression system,
and further optionally wherein the at least two doses comprise the same antigen cassette and/or wherein the at least two doses comprises a priming dose and at least one boosting dose, optionally wherein:
(a) the at least two doses are administered on days 1 and day 28 or later, optionally wherein day 28 or later comprises day 29 or later;
(b) the at least two doses are administered on days 1 and on or after week 4;
(c) the at least two doses are administered on days 1 and between day 28 to 113; or
(d) the at least two doses are administered on days 1 and day 113 or later.

13-20. (canceled)

21. The method of claim 12, wherein the method further comprises administration of a chimpanzee adenovirus (ChAdV)-based expression system,
wherein the composition for delivery of the ChAdV-based expression system comprises:
the ChAdV-based expression system, wherein the ChAdV-based expression system comprises a viral particle comprising a ChAdV vector, wherein the ChAdV vector comprises:
(a) a ChAdV backbone, wherein the ChAdV backbone comprises:
(i) at least one promoter nucleotide sequence, and
(ii) at least one polyadenylation (poly(A)) sequence; and
(b) an antigen cassette, wherein the antigen cassette is inserted into the vector backbone, and wherein the antigen cassette comprises at least one SARS-CoV-2 derived nucleic acid sequence encoding an immunogenic polypeptide;
optionally wherein the ChAdV-based expression system is administered as a priming dose, optionally wherein the antigen cassette of the ChAdV-based expression system is the same as the antigen cassette of the self-amplifying alphavirus-based expression system.

22. (canceled)

23. (canceled)

24. The composition of claim 40, wherein the antigen expression system comprises a chimpanzee adenovirus (ChAdV)-based expression system,
wherein the composition for delivery of the ChAdV-based expression system comprises:
a viral particle comprising a ChAdV vector, wherein the ChAdV vector comprises:
(a) a ChAdV backbone, wherein the ChAdV backbone comprises:
(i) at least one promoter nucleotide sequence, and
(ii) at least one polyadenylation (poly(A)) sequence; and
(b) the antigen cassette, wherein the antigen cassette is inserted into the vector backbone,
(ii) optionally, a second promoter nucleotide sequence operably linked to the SARS-CoV-2 derived nucleic acid sequence; and
(iii) optionally, at least one MHC class II epitope-encoding nucleic acid sequence;
(iv) optionally, at least one nucleic acid sequence encoding a GPGPG amino acid linker sequence (SEQ ID NO: 56); and
(v) optionally, at least one second poly(A) sequence, wherein the second poly(A) sequence is a native poly(A) sequence or an exogenous poly(A) sequence to the vector backbone, optionally wherein the exogenous poly(A) sequence comprises an SV40 poly(A) signal sequence or a Bovine Growth Hormone (BGH) poly(A) signal sequence, and
wherein the cassette is operably linked to the at least one promoter nucleotide sequence and the at least one poly(A) sequence, and wherein the composition comprises 1×10¹² or less of the viral particles;
optionally wherein the composition is further formulated in a pharmaceutical composition comprising a pharmaceutically acceptable carrier.

25. The composition of claim 24, wherein the composition for delivery of the ChAdV-based expression system comprises:
(a) at least 1×10¹¹ of the viral particles;
(b) between 1×10¹¹ and 1×10¹²; or
(c) 1×10¹¹, 3×10¹¹, or 1×10¹² of the viral particles;
optionally wherein the viral particles are at a concentration of 5×10¹¹ vp/mL, and further optionally wherein the ChAdV backbone comprises at least nucleotides 2 to 36,518 of the sequence set forth in SEQ ID NO:1, wherein the nucleotides 2 to 36,518 lack: (1) nucleotides 577 to 3403 of the sequence shown in SEQ ID NO:1 corresponding to an E1 deletion; (2) nucleotides 27,125 to 31,825 of the sequence shown in SEQ ID NO:1 corresponding to an E3 deletion; and (3) optionally nucleotides 34,916 to 35,642 of the sequence shown in SEQ ID NO:1 corresponding to a partial E4 deletion; optionally wherein the antigen cassette is inserted within the E1 deletion.

26-29. (canceled)

30. A method for stimulating an immune response in a subject, the method comprising administering to the subject the composition for delivery of the ChAdV-based expression system of claim 24, optionally wherein:
(1) the ChAdV-based expression system is administered as a priming dose; and/or
(2) the method further comprises administration of a composition for delivery of a self-amplifying alphavirus-based expression system,
wherein the composition for delivery of the self-amplifying alphavirus-based expression system comprises the self-amplifying alphavirus-based expression system, wherein the self-amplifying alphavirus-based expression system comprises one or more vectors, wherein the one or more vectors comprises:
(a) an RNA alphavirus backbone, wherein the RNA alphavirus backbone comprises:
(i) at least one promoter nucleotide sequence, and
(ii) at least one polyadenylation (poly(A)) sequence; and
(b) an antigen cassette, wherein the antigen cassette is inserted into the vector backbone, and wherein the antigen cassette comprises at least one SARS-CoV-2 derived nucleic acid sequence encoding an immunogenic polypeptide:
optionally wherein the antigen cassette of the ChAdV-based expression system is the same as the antigen cassette of the self-amplifying alphavirus-based expression system.

31-37. (canceled)

38. A method for stimulating an immune response in a subject, the method comprising administering to the subject a composition for delivery of a self-amplifying alphavirus-based expression system and administering to the subject a composition for delivery of a chimpanzee adenovirus (ChAdV)-based expression system, and
wherein either:
a. the composition for delivery of the ChAdV-based expression system comprises the ChAdV-based expression system, wherein the ChAdV-based expression system comprises a viral particle comprising a ChAdV vector, and wherein the composition comprises 1×10¹² or less of the viral particles,
b. the composition for delivery of the self-amplifying alphavirus-based expression system comprises the self-amplifying alphavirus-based expression system, wherein the self-amplifying alphavirus-based expression system comprises one or more vectors, and wherein the composition comprises at least 10 μg of each of the one or more vectors, or
c. the composition for delivery of the ChAdV-based expression system comprises the ChAdV-based expression system, wherein the ChAdV-based expression system comprises a viral particle comprising a ChAdV vector, and wherein the composition comprises 1×10¹² or less of the viral particles and wherein the composition for delivery of the self-amplifying alphavirus-based expression system comprises the self-amplifying alphavirus-based expression system, wherein the self-amplifying alphavirus-based expression system comprises one or more vectors, and wherein the composition comprises at least 10 μg of each of the one or more vectors;
optionally wherein the composition for delivery of the ChAdV-based expression system is administered as a priming dose and the composition for delivery of the self-amplifying alphavirus-based expression system is administered as one or more boosting doses.

39. (canceled)

40. A composition for delivery of an antigen expression system, comprising:
the antigen expression system, wherein the antigen expression system comprises:
(a) optionally, one or more vectors, the one or more vectors comprising:
a vector backbone, wherein the vector backbone comprises:
(i) at least one promoter nucleotide sequence, and
(ii) at least one polyadenylation (poly(A)) sequence; and
(1b) an antigen cassette, optionally wherein the antigen cassette is inserted into the vector backbone when present, and wherein the antigen cassette comprises:
(i) a SARS-CoV-2 derived nucleic acid sequence encoding an immunogenic polypeptide, wherein the immunogenic polypeptide comprises a SARS-CoV-2 variant Spike protein corresponding to a B.1.1.529 SARS-CoV-2 isolate optionally comprising the Spike polypeptide sequence as set forth in SEQ ID NO: 27974 and/or is encoded by nucleotides 9714-13,526 of the nucleotide sequence set forth in SEQ ID NO: 27976, or a SARS-CoV-2 variant Spike protein corresponding to a B.1.1.529 BA5 SARS-CoV-2 subvariant optionally comprising the Spike polypeptide sequence as set forth in SEQ ID NO: 27978 and/or encoded by SEQ ID NO: 27980,
optionally wherein (a) the B.1.1.529 isolate Spike protein or fragment comprises a mutation selected from the group consisting of: a Spike R679 mutation, a Spike R680 mutation, a Spike R682 mutation, a Spike K983P mutation, a Spike V984P mutation, and combinations thereof with reference to the Spike polypeptide sequence as set forth in SEQ ID NO:27977 or (b) the B.1.1.529 BA5 subvariant Spike protein or fragment comprises a mutation selected from the group consisting of: a Spike R677 mutation, a Spike R678 mutation, a Spike R680 mutation, a Spike K981P mutation, a Spike V982P mutation, and combinations thereof with reference to the Spike polypeptide sequence as set forth in SEQ ID NO: 27979, and
optionally wherein the antigen cassette further comprises at least one additional SARS-CoV-2 derived nucleic acid sequence encoding at least one additional immunogenic polypeptide, wherein the at least one additional immunogenic polypeptide comprises:
at least one MHC class I epitope comprising a polypeptide sequence as set forth in Table A,
at least one MHC class II epitope comprising a polypeptide sequence as set forth in Table B,
at least one MHC class I epitope comprising a polypeptide sequence as set forth in Table C, optionally wherein the at least one MHC I epitope is present in a concatenated polypeptide sequence as set forth in SEQ ID NO:57 or SEQ ID NO:58,
at least one polypeptide sequence as set forth in Table 6, or an epitope-containing fragment thereof, optionally wherein the at least one polypeptide sequence is present in a concatenated polypeptide sequence as set forth in SEQ ID NO:92,
at least one polypeptide sequence as set forth in Table 9A, Table 9B, or Table 9C, or an epitope-containing fragment thereof, optionally wherein the at least one polypeptide sequence is present in a concatenated polypeptide comprising each of the sequences set forth in Table 9A, Table 9B, or Table 9C, optionally wherein the concatenated polypeptide comprises the order of sequences set forth in Table 9A, Table 9B, or Table 9C,
at least one MHC class I epitope comprising a polypeptide sequence as set forth in Table A and/or Table C or MHC class II epitope comprising a polypeptide sequence as set forth in Table B, wherein the encoded SARS-CoV-2 immunogenic polypeptide is conserved between SARS-CoV-2 and a Coronavirus species and/or sub-species other than SARS-CoV-2, optionally wherein the Coronavirus species and/or sub-species other than SARS-CoV-2 is Severe acute respiratory syndrome (SARS) and/or Middle East respiratory syndrome (MERS),
one or more validated epitopes and/or at least 4, 5, 6, or 7 predicted epitopes, wherein at least 85%, 90%, or 95% of a population carries at least one HLA validated to present at least one of the one or more validated epitopes and/or at least one HLA predicted to present each of the at least 4, 5, 6, or 7 predicted epitopes,
a SARS-CoV-2 Spike protein or an epitope-containing fragment thereof corresponding to an isolate other than a B.1.1.529 SARS-CoV-2 isolate, optionally comprising a Spike polypeptide sequence as set forth in SEQ ID NO:59, optionally wherein the Spike polypeptide comprises a D614G mutation with reference to SEQ ID NO:59, and optionally wherein the Spike polypeptide is encoded by the nucleotide sequence shown in SEQ ID NO:79, SEQ ID NO:83, SEQ ID NO:85, or SEQ ID NO:87,
a SARS-CoV-2 modified Spike protein comprising a mutation selected from the group consisting of: a Spike R682 mutation, a Spike R683 mutation, a Spike R685 mutation, a Spike R815 mutation, a Spike K986P mutation, a Spike V987P mutation, and combinations thereof with reference to the Spike polypeptide sequence as set forth in SEQ ID NO:59, and optionally wherein the modified Spike protein comprises a polypeptide sequence as set forth in SEQ ID NO:60 or SEQ ID NO:90 or an epitope-containing fragment thereof,
a SARS-CoV-2 Membrane protein comprising a Membrane polypeptide sequence as set forth in SEQ ID NO:61 or an epitope-containing fragment thereof,
a SARS-CoV-2 Nucleocapsid protein comprising a Nucleocapsid polypeptide sequence as set forth in SEQ ID NO:62 or an epitope-containing fragment thereof,
a SARS-CoV-2 Envelope protein comprising an Envelope polypeptide sequence as set forth in SEQ ID NO:63 or an epitope-containing fragment thereof,
a variant of any of the above comprising a mutation found in 1% or greater of SARS-CoV-2 subtypes, optionally wherein the variant comprises a SARS-CoV-2 variant shown in Table 1,
or combinations thereof, and
wherein the immunogenic polypeptide optionally comprises a N-terminal linker and/or a C-terminal linker; or
(2b) an antigen cassette, optionally wherein the antigen cassette is inserted into the vector backbone when present, and wherein the antigen cassette comprises:
(i) three SARS-CoV-2 derived nucleic acid sequences encoding immunogenic polypeptides, wherein the immunogenic polypeptides comprises:
(A) a SARS-CoV-2 derived nucleic acid sequence encoding an immunogenic polypeptide, wherein the immunogenic polypeptide comprises a SARS-CoV-2 variant Spike protein corresponding to a B.1.1.529 SARS-CoV-2 isolate optionally comprising the Spike polypeptide sequence as set forth in SEQ ID NO: 27974 and/or is encoded by nucleotides 9714-13,526 of the nucleotide sequence set forth in SEQ ID NO: 27976, or a SARS-CoV-2 variant Spike protein corresponding to a B.1.1.529 BA5 SARS-CoV-2 subvariant optionally comprising the Spike polypeptide sequence as set forth in SEQ ID NO: 27978 and/or encoded by SEQ ID NO: 27980, optionally wherein (a) the B.1.1.529 isolate Spike protein or fragment comprises a mutation selected from the group consisting of: a Spike R679 mutation, a Spike R680 mutation, a Spike R682 mutation, a Spike K983P mutation, a Spike V984P mutation, and combinations thereof with reference to the Spike polypeptide sequence as set forth in SEQ ID NO:27977 or (b) the B.1.1.529 BA5 subvariant Spike protein or fragment comprises a mutation selected from the group consisting of: a Spike R677 mutation, a Spike R678 mutation, a Spike R680 mutation, a Spike K981P mutation, a Spike V982P mutation, and combinations thereof with reference to the Spike polypeptide sequence as set forth in SEQ ID NO: 27979;
(B) at least one polypeptide sequence as set forth in Table 9C, or an epitope-containing fragment thereof, optionally wherein the at least one polypeptide sequence is present in a concatenated polypeptide comprising each of the sequences set forth in Table 9C, optionally wherein the concatenated polypeptide comprises the order of sequences set forth in Table 9C, and
(C) a SARS-CoV-2 Nucleocapsid protein, optionally comprising a Nucleocapsid polypeptide sequence as set forth in SEQ ID NO:62 or an epitope-containing fragment thereof,
wherein each of the SAR-CoV-2 SARS-CoV-2 derived nucleic acid sequences comprises;
(I) optionally, a 5′ linker sequence, and
(II) optionally, a 3′ linker sequence;
(ii) optionally, a second promoter nucleotide sequence operably linked to one or more of the three SARS-CoV-2 derived nucleic acid sequences; and
(iii) optionally, at least one MHC class II epitope-encoding nucleic acid sequence;
(iv) optionally, at least one nucleic acid sequence encoding a GPGPG amino acid linker sequence (SEQ ID NO:56); and
(v) optionally, at least one second poly(A) sequence, wherein the second poly(A) sequence is a native poly(A) sequence or an exogenous poly(A) sequence to the vector backbone optionally wherein the exogenous poly(A) sequence comprises an SV40 poly(A) signal sequence or a Bovine Growth Hormone (BGH) poly(A) signal sequence, or
(3b) an antigen cassette, wherein the antigen cassette is inserted into the vector backbone such that the antigen cassette is operably linked to the at least one promoter nucleotide sequence, wherein the backbone comprises an alphavirus vector, wherein the alphavirus vector is a Venezuelan equine encephalitis virus vector, and wherein the vector backbone comprises: (i) a subgenomic promoter nucleotide sequence, and (ii) at least one polyadenylation (poly(A)) sequence; and
wherein the antigen cassette comprises:
(i) three SARS-CoV-2 derived nucleic acid sequences encoding immunogenic polypeptides, wherein the immunogenic polypeptides comprises:
(A) a SARS-CoV-2 derived nucleic acid sequence encoding an immunogenic polypeptide, wherein the immunogenic polypeptide comprises a SARS-CoV-2 variant Spike protein corresponding to a B.1.1.529 SARS-CoV-2 isolate optionally comprising the Spike polypeptide sequence as set forth in SEQ ID NO: 27974 and/or is encoded by nucleotides 9714-13,526 of the nucleotide sequence set forth in SEQ ID NO: 27976, or a SARS-CoV-2 variant Spike protein corresponding to a B.1.1.529 BA5 SARS-CoV-2 subvariant optionally comprising the Spike polypeptide sequence as set forth in SEQ ID NO: 27978 and/or encoded by SEQ ID NO: 27980, optionally wherein (a) the B.1.1.529 isolate Spike protein or fragment comprises a mutation selected from the group consisting of: a Spike R679 mutation, a Spike R680 mutation, a Spike R682 mutation, a Spike K983P mutation, a Spike V984P mutation, and combinations thereof with reference to the Spike polypeptide sequence as set forth in SEQ ID NO:27977 or (b) the B.1.1.529 BA5 subvariant Spike protein or fragment comprises a mutation selected from the group consisting of: a Spike R677 mutation, a Spike R678 mutation, a Spike R680 mutation, a Spike K981P mutation, a Spike V982P mutation, and combinations thereof with reference to the Spike polypeptide sequence as set forth in SEQ ID NO: 27979;
(B) at least one polypeptide sequence as set forth in Table 9C, or an epitope-containing fragment thereof, optionally wherein the at least one polypeptide sequence is present in a concatenated polypeptide comprising each of the sequences set forth in Table 9C, optionally wherein the concatenated polypeptide comprises the order of sequences set forth in Table 9C, and
(C) a SARS-CoV-2 Nucleocapsid protein, optionally comprising a Nucleocapsid polypeptide sequence as set forth in SEQ ID NO:62 or an epitope-containing fragment thereof;
(ii) a second subgenomic promoter nucleotide sequence operably linked to at least one of the three SARS-CoV-2 derived nucleic acid sequences; and
(iii) optionally, at least one MHC class II epitope-encoding nucleic acid sequence;
(iv) optionally, at least one nucleic acid sequence encoding a GPGPG amino acid linker sequence (SEQ ID NO:56); and
(v) optionally, at least one second poly(A) sequence, wherein the second poly(A) sequence is a native poly(A) sequence or an exogenous poly(A) sequence to the vector backbone optionally wherein the exogenous poly(A) sequence comprises an SV40 poly(A) signal sequence or a Bovine Growth Hormone (BGH) poly(A) signal sequence; or
(4b) an antigen cassette, wherein the antigen cassette is inserted into the vector backbone such that the antigen cassette is operably linked to the at least one promoter nucleotide sequence, wherein the backbone comprises an alphavirus vector, wherein the alphavirus vector is a Venezuelan equine encephalitis virus vector, and wherein the vector backbone comprises: (i) a subgenomic promoter nucleotide sequence, and (ii) at least one polyadenylation (poly(A)) sequence; and
wherein the antigen cassette comprises, in order from 5′ to 3′:
(i) a nucleotide sequence encoding a SARS-CoV-2 Nucleocapsid protein comprising a Nucleocapsid polypeptide sequence as set forth in SEQ ID NO:62;
(ii) a 2A ribosome skipping sequence element;
(iii) a nucleotide sequence encoding a concatenated polypeptide comprising each of the sequences set forth in Table 9C and in the order of sequences set forth in Table 9C;
(iv) a nucleotide sequence encoding at least one universal MHC class II epitope, optionally further encoding one or more GPGPG amino acid linker sequences (SEQ ID NO:56) at the 5′ terminus, 3′ terminus, and/or in between concatenated universal MHC class II epitope sequences;
(v) a second subgenomic promoter nucleotide sequence; and
(vi) a nucleotide sequence encoding a B.1.1.529 SARS-CoV-2 isolate optionally comprising the Spike polypeptide sequence as set forth in SEQ ID NO: 27974 and/or is encoded by nucleotides 9714-13,526 of the nucleotide sequence set forth in SEQ ID NO: 27976, or a SARS-CoV-2 variant Spike protein corresponding to a B.1.1.529 BA5 SARS-CoV-2 subvariant optionally comprising the Spike polypeptide sequence as set forth in SEQ ID NO: 27978 and/or encoded by SEQ ID NO: 27980.

41. An antigen-based vaccine comprising:
(i) a SARS-CoV-2 variant Spike protein corresponding to a B.1.1.529 SARS-CoV-2 isolate optionally comprising the Spike polypeptide sequence as set forth in SEQ ID NO: 27974 and/or is encoded by nucleotides 9714-13,526 of the nucleotide sequence set forth in SEQ ID NO: 27976, or a SARS-CoV-2 variant Spike protein corresponding to a B.1.1.529 BA5 SARS-CoV-2 subvariant optionally comprising the Spike polypeptide sequence as set forth in SEQ ID NO: 27978 and/or encoded by SEQ ID NO: 27980,
optionally wherein (a) the B.1.1.529 isolate Spike protein or fragment comprises a mutation selected from the group consisting of: a Spike R679 mutation, a Spike R680 mutation, a Spike R682 mutation, a Spike K983P mutation, a Spike V984P mutation, and combinations thereof with reference to the Spike polypeptide sequence as set forth in SEQ ID NO:27977 or (b) the B.1.1.529 BA5 subvariant Spike protein or fragment comprises a mutation selected from the group consisting of: a Spike R677 mutation, a Spike R678 mutation, a Spike R680 mutation, a Spike K981P mutation, a Spike V982P mutation, and combinations thereof with reference to the Spike polypeptide sequence as set forth in SEQ ID NO: 27979, and
optionally wherein the antigen-based vaccine further comprises at least one additional SARS-CoV-2 derived immunogenic polypeptide, wherein the additional immunogenic polypeptide comprises:
at least one MHC class I epitope comprising a polypeptide sequence as set forth in Table A,
at least one MHC class II epitope comprising a polypeptide sequence as set forth in Table B,
at least one MHC class I epitope comprising a polypeptide sequence as set forth in Table C, optionally wherein the at least one MHC I epitope is present in a concatenated polypeptide sequence as set forth in SEQ ID NO:57 or SEQ ID NO:58,
at least one polypeptide sequence as set forth in Table 6, or an epitope-containing fragment thereof, optionally wherein the at least one polypeptide sequence is present in a concatenated polypeptide sequence as set forth in SEQ ID NO:92,
at least one polypeptide sequence as set forth in Table 9A, Table 9B, or Table 9C, or an epitope-containing fragment thereof, optionally wherein the at least one polypeptide sequence is present in a concatenated polypeptide comprising each of the sequences set forth in Table 9A, Table 9B, or Table 9C, optionally wherein the concatenated polypeptide comprises the order of sequences set forth in Table 9A, Table 9B, or Table 9C,
at least one MHC class I epitope comprising a polypeptide sequence as set forth in Table A and/or Table C or MHC class II epitope comprising a polypeptide sequence as set forth in Table B, wherein the encoded SARS-CoV-2 immunogenic polypeptide is conserved between SARS-CoV-2 and a Coronavirus species and/or sub-species other than SARS-CoV-2, optionally wherein the Coronavirus species and/or sub-species other than SARS-CoV-2 is Severe acute respiratory syndrome (SARS) and/or Middle East respiratory syndrome (MERS),
one or more validated epitopes and/or at least 4, 5, 6, or 7 predicted epitopes, wherein at least 85%, 90%, or 95% of a population carries at least one HLA validated to present at least one of the one or more validated epitopes and/or at least one HLA predicted to present each of the at least 4, 5, 6, or 7 predicted epitopes,
a SARS-CoV-2 Spike protein comprising a Spike polypeptide sequence as set forth in SEQ ID NO:59 or an epitope-containing fragment thereof, optionally wherein the Spike polypeptide comprises a D614G mutation with reference to SEQ ID NO:59, and optionally wherein the Spike polypeptide is encoded by the nucleotide sequence shown in SEQ ID NO:79, SEQ ID NO:83, SEQ ID NO:85, or SEQ ID NO:87,
a SARS-CoV-2 modified Spike protein comprising a mutation selected from the group consisting of: a Spike R682 mutation, a Spike R683 mutation, a Spike R685 mutation, a Spike R815 mutation, a Spike K986P mutation, a Spike V987P mutation, and combinations thereof with reference to the Spike polypeptide sequence as set forth in SEQ ID NO:59, and optionally wherein the modified Spike protein comprises a polypeptide sequence as set forth in SEQ ID NO:60 or SEQ ID NO:90 or an epitope-containing fragment thereof,
a SARS-CoV-2 Membrane protein comprising a Membrane polypeptide sequence as set forth in SEQ ID NO:61 or an epitope-containing fragment thereof,
a SARS-CoV-2 Nucleocapsid protein comprising a Nucleocapsid polypeptide sequence as set forth in SEQ ID NO:62 or an epitope-containing fragment thereof,
a SARS-CoV-2 Envelope protein comprising an Envelope polypeptide sequence as set forth in SEQ ID NO:63 or an epitope-containing fragment thereof,
a variant of any of the above comprising a mutation found in 1% or greater of SARS-CoV-2 subtypes, optionally wherein the variant comprises a SARS-CoV-2 variant shown in Table 1,
or combinations thereof, and
wherein the immunogenic peptide optionally comprises a N-terminal linker and/or a C-terminal linker
(ii) optionally, at least one MHC class II antigen; and
(iii) optionally, at least one GPGPG amino acid linker sequence (SEQ ID NO:56);
further optionally wherein the antigen expression system comprises:
(a) the nucleotide sequence as set forth in SEQ ID NO:27976, optionally wherein the nucleotides encoding a SARS-CoV-2 variant Spike protein corresponding to the B.1.1.529 SARS-CoV-2 isolate are substituted with the nucleotides encoding a SARS-CoV-2 variant Spike protein corresponding to a B.1.1.529 BA5 SARS-CoV-2 subvariant; or
(b) the nucleotide sequence as set forth in nucleotides 7,571 to 13,526 of SEQ ID NO:27976, optionally wherein the nucleotides encoding a SARS-CoV-2 variant Spike protein corresponding to the B.1.1.529 SARS-CoV-2 isolate are substituted with the nucleotides encoding a SARS-CoV-2 variant Spike protein corresponding to a B.1.1.529 BA5 SARS-CoV-2 subvariant; or
(c) a self-amplifying alphavirus-based expression system comprising a vector selected from the group of sequences consisting of: SEQ ID NO: 27983, SEQ ID NO:27981, SEQ ID NO:27982, SEQ ID NO: 27976, and SEQ ID NO: 27976 with Spike encoding sequences substituted with the sequence set forth in SEQ ID NO:27980.

42-48. (canceled)
